SEQUENCE LISTING (419 sequences)

SEQ ID NO: 1 - DNA, 4067 bp, Homo sapiens
  misc_feature 1..4067: note = Human AAVS1
  source 1..4067: mol_type = genomic DNA
gaattcctaactgccccggggcagtctgctattcatcccctttacgcggtgctacacacacttgctagtatgccgtggggacccctccggcctgtagactccatttcccagcattccccggaggaggccctcatctggcgatttccactgggggcctcggagctgcggacttcccagtgtgcatcggggcacagcgactcctggaagtggccacttctgctaatggactccatttcccaggctcccgctacctgcccagcacaccctggggcatccgtgacgtcagcaagccgggcggggaccggagatccttggggcggtggggggccagcggcagttcccaggcggcccccggggcgggcgggcgggcgggtggtggcggcggttggggctccgggcgcgtcgctcgctcgctcgctgggcgggcgggcggtgcgatgtccggagaggatggccggcggctggcccgggggcggcggcgcggctgcccgggagcggcgacgggagcagctgcggcagtggggcgcgggcgggcgccgagcctggccccggagagcgccgcgcccgcaccgtccgcttcgagcgcgccgccgagttcctggcggcctgtgcgggcggcgacctggacgaggcgcgtctgatgctgcgcgccgccgaccctggccccggcgccggagctcgaccccgccggccgccgcccgcccgcgccgtgctggactccaccaacgccgacggtatcagcgccctgcaccaggtcagcgccccccgcggcgtctcccggggccaggtccaccctctgcgccacctggggcatcctccttccccgttgccagtctcgatccgccccgtcgttactggccctgggtttncaccctatgctgacaccccgttccagtccccttaccattcccttcgaccaccccacttccgaattggagcgcttcaactggctgggctagcactctgtgtgacactctgaagctctacattcccttcgacctactctcttcgattggagtcgctttaactggccctggctttggcagcctgtgctgacccatcgagtcctccttaccatccctccctcgacttcccctcttccgatgttgagcccctccagccggtcctggactttgtctccttccctgccctgccctctcctgaacctgagccagctcccatagctcaggtctggtctatctgcctggccctggccattgtcactttgcgctgccctcctctcgcccccgagtgcccttgctgtgccgccggaactctgccctctaacgctgccgtgccgtctctctcctgagtccggaccactttgagctctactggcttctgcgcgcctctggcccactgtttccccttcccaggcaggtcctgctttctctgaccagcattctctcccctgggcctgtgccgctttctgtctgcagcttgtggcctgggtcacctctacggctggcccaagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtgttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggcttagcacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagcccccatccttaggcctcctccttcctagtctcctgatattcgtctaacccccacctcctgttaggcagattccttatctggtgacacacccccatttcctggagccatctctctccttgccagaacctctaaggtttgcttacgatggagccagagaggatcctgggagggagacttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagnccccaatttatattgttcctccgtgcgtcagttttacctgtgagataaggccagtagccacccccgtcctggcagggctgtggtgaggaggggggtgtccgtgtggaaaactccctttgtgagaatggtgcgtcctaggtgttcaccaggtcgtggccgcctctactccctttctctttctccatccatccttctttccttaaagagcccccagtgctatctggacatattcctccgcccagagcagggtccgcttccctaaggccctgctctgggcttctgggtttgagtccttgcaagcccaggagagcgctagcttccctgtcccccttcctcgtccaccatctcatgccctggctctcctgccccttcctacaggggttcctggctctgctcttcagactgagccccgttcccctgcatccccgttcccctgcatcccccttcccctgcatcccccagagccccaggccacctacttggcctggaaccccacgagaggccaccccagccctgtctaccaggctgaccttttgggtgattctcctccaactgtggggtgactgcttgggcaaactcactcttcggggtatcccaggaggcctggagcattggggtgggctggggttcagagaggagggattccctccaggttacgtggccaagaagcaggggagctgggtttgggtcaggctgggtgtggggtgaccagcttatgctgtttgcccaggacagcctagttttagcgctgaaaccctcagtcctaggaaaacagggatggttggtcactgtctctgggtgactcttgattcccggccagtttctccacctggggctgtgtttctcgtcctgcatccttctccaggcaggtccccaagcatcgcccccctggctgttcccaagttcttaggtaccccacgtgggtttatgaaccacttggtgaggctggtaccctgcccccattcctgcaccccaattgccttagtggctagggggttgggggctagagtaggaggggctggagccaggattcttagggctgaacagagccgagctgggggcctgggctcctgggtttgagagaggaggggctggggcctggactcctgggtccgagggaggaggggctggggcctggactcctgggtctgagggtggagggactgggggcctggactcctgggtccgagggaggaggggctggggcctggactcgtgggtctgagggaggaggggtcgggggcctggacttctgggtcttagggaggcggggctgggcctggacccctgggtctgaatggggagaggctgggggcctggactccttcatctgagggcggaagggctggggcctggcctcctgggttgaatggggaggggttgggcctggactctggagtccctggtgcccaggcctcaggcatctttcacagggatgcctgtac

SEQ ID NO: 2 - DNA, 44232 bp, synthetic construct
  misc_feature 1..44232: note = Synthetic: CHO AAVS1-Like Sequence
  source 1..44232: mol_type = other DNA
ccagcacccacatggtggctcacaactgtccgtaactccagttccagaggatctgatgccctcttctgtctcccgcgagcacctggcacacacgtgatgcacacttaaacacatgcaagcaaaccatcagacacataacttttttttccaattttttaaagatttagttattattatttacttaataaatatttattatatttattacatatacagtttctgcctacatgccagcagagggcaccagattgaattgtagatggttgtgagccaccatgtggttgctgggaattgaactcaggacccctggaagagcagtcagtgctcttaacctctgagccatctctccagcccctccatttttttttttttaaataaagaaatgtaatgtcctaagtggggcttagagagtggaagcagataaagaaagatggagttaagaattttaagaagccagttggcggttgtgcatgccagcactcaggaggcagaggcaggtggatggatctctatgagttcgaggccagcctggtctacagagagagagttccaggacagacttctccaaagctacagagaaaccctgtctgaacccaccacgaccaccacaaagaaaaaaaggatttcaagaggagagccaggtttatagcaagagagaaagttgtgaactaatgcccagggcttagtgtggcctacctctgggctgggtctctctctgaacacagggtggagctgccccgggaggaagaagcggctccgtacagtcccgaattctacagtggctgggagcctcccgccactgacccgcagggccgcgcctgggaggacccggtggaaaaacagctacagcatgagaagaggcgcaggcaggtgaggcagggttgccgggggagcactgggctccccgtttctgcacaacatgggcgagcaggacgtctgaggtctagcctgcctgaccccaagctctctctcttcccgcagcaaagcgccccccagatcgctgtcaatgggtgagtgaccgctgcagggtggccagggatggggttgggaggactgagtcccggggtcaccccggctctgactccgaccctcccccttttttcttgtctttttttttttttttttttttttttaaacctctgccttcccggctctttgcaggtgggtgaggtggtgaggaggcggggctggggtgggggtgggggaggagccaggagggagggggggaggagcccagaactctgggtccaagggaagagggaaaggaggcttagtttgctgaagctatgagagttaggggctgaaagtgggtgggtctaaaggcttggaccccacacccccacccccggcatcctcaaaagattgaaaaggtgcagtttggtgttctaggacctgggagagcaccatgcttgagtccccagagcacagagcactgggtgtcagagaaaaaaaaaaaatggagaccaaaaagcagggttgggacttccgaggattcagggacaagtttgaggaaacgtgagaaagtgctggcatccctggaccactaactgaggtgggacttccggcttcctaatgcgcaaaggaatagcacgtactgagcaaactggaatgctcccagggctgaaagaatggaggaaattgaaggtcaaggcacggactcctgcctaggtccctgggaaggaaagaactagggacctaaatttacagttctaccaaactatggaagctgagggctgcaggtccaggtgaggaagtgatggagagggggtcacagccctaggatccttggggaaataggggccaggagtggagggcgtggatgtggcttgagaacaaaatgatagacttggaggagaggaattgggggcctaggtgagagccccagcagagggtctcagcagggacggcatactgggagctgtcagtcccacacatggggcgccgaggccctgaagagtcccctcctcccttccacaggtaggcctgatccgggatgaggtctctcttgctgggggcgccagagctaatcgtcccccaggctgcctggtgctgcagggccctcttgtctgtctgtctgcttctgaatcttgggctcagcacctgcaagctgtttactcgccttctctggctgtaatttctttgcctggaagggtgaggactctctggcgctgtaaggggcttgcaaagagctcagtgccgtgactcagcctgagttcaaatccagctgcatgaagaacagtacagagtgaccctgacaagggcagcctagggccagctcagtcacacctttctctttcttgtgcactggccgttactacagtatccctcggttccttcatatagaaagagaaatagtgagccgggcagtggtggcgcacacctttaatcccagcacttgggaggcaaaggcaggtggacctctgtgagttcaagaccagcctggtctacaagagctagttccaggatagtctccaaagccacagagaaaccctgtctcgaaaaaccaaaaaagaaaaaagaaagaaagagaaatagtgagaccggcagtggtggtgcacgtctttagtcccagcactggggaggcagaggcagccggatttctgtgagttcaaggatagactggtctacagagtgagttccaagacagccagaactaaacagtgaaaccctgtcttggaaaaaaaaaaaagtgaaataatggccatattctggtgatggtgtaggcctgtggtcccagctactcagagacatgaagcaggagaataaaaatcaaggcctgctttgactacaaagtgagcttcaaaggccagcctgggcaaagcaacaaggccttgcctcaaaatgaaaaaataaaaataaaagaggctggagaaatggcttagtggttaagagtactggccgctcttccaggggaccagggttcaattcccagcacccagacatacagcagctcacaactccagtttcagggaatccggtgttctctctggtctctgtaggcaccaggcactcaagttgtgcagacataaaataacacagagggctgggctggggctcagtggcaggcatttgcccagaatcccccagtaaagacatagctcagtgaatccagagctgaggggctgggcgtatattaatggtggaatccttgcctagaattcaaccagcgaagggctgtggccgtggctcggctgtagaaccctgtcctggtatctaccatgaagggctgggacatggctcagagataaaacacttgcctagactctaccgctgagagcctggggtgtggatcagtggacagtgcccgcctagcatgcacaaggcccctgggttcaatcccctgtaccacaaaaaaaagggggggtggagggagggtaagagtgagatctcaggagaaggaaggaaccaaattcatggaactacaagggaactccaggagaatcgaagcgtttctggcgtacgttgctgtgtaagcacaagggtcggctatttttgcaccctgttcattatcctagcgggtgatgggaatagatctgctgtctctagccgattcctcatgatcctcactgatgaaaatgcaggtgaggggctggagagattaagaacactgtctgctctggcactggacctaggttcattcagctccccacagcacatggtggcccacaaatatctgtaactccagctctaagaacccaggtctaggacaccctctcctggactctgtggctactgcacacaggtgatgcacatacacacacatgcatgcaggcaacacacacacacacacacacacacacacacacacacacacaatgcatgtgaacgactggggatgaagctcggaagctaagcacttccctggcatgcacgggccctgggttcaatccccagcaccccataatgaattaaatcgttatcatgatacggtgtgtttactgcatggtgccaggcaaggaaatgagctaactccattcaagctgtgactccagtgtcaagcctgtattaacatattaacctgggcctctgctctgaccccctgcttggctctaaccccacctcacaccttagagtccagaccagcagggctggctacctcctaatctcctgctggtttctttctccccagtcatcaagatccagacctggaagccgccgagctagaagagagagccagaaagtgggttctgtgtaactatgacttccaggcccgaaatagcagcgagctgtctgtcaagcacggagatgtgttggaggttagcggtgtggggggcctgagaccctgaaattggtcaatttagccctaggtatagaaccggagcgtgaattctctccttatacgccacctaggtcctggatgacaggcgcaagtggtggaaggttcgggaccatcagggacaggagggttatgtaccctataacatcctgacaccccaccctggacctcaggtgcaccgcagccaaagtcctgcaggaaacctagtaagtcggcgtgttcttgcttcttcggggagaaaggggggcaagatcctaggtcctggggatgaggacagagaaaatcaggtgtgaaggttgctgtttggaaaggggggggggtggtcagatgtttattgggaaaggagctggaagcctctcttcattcccttccaggagacgagtactcctcctcccccacccgcaccagctccagcccctgctcaggtgcgaccccactgggacagttgcgacagtctcaacaatttggaccccagcgagaagggtgagtggtggagcgtcactctgggaagtgatccttgtcttcgcttttcaggctccaccctgggcaccctagcggctcccagccccctgaccccagaacccctgagcgcgcactcccctccgcccccccccctcacggtttcgcttctgcagagaaattctcccagatgctcagtgtcaatgaggagctgcaggcgcgccttgcgcagggccgttcgggtcccagccgggtagccccgggaccccgcgccccggagcctcagctcagcccgcgctctgaggcctcggtggtccgtgcctggctgcagaccaagggctttagctcggggtgagtggggctccccccggggctagtctgaagagacctgtgcttgaactgaaaggcgaggttcccattggtccaggggtgggggcgtggaaactgtggagcaggcccaaattgcaacgcccaatgcccagggacaggctccaaacggaggccacaggaaaggaagtcccatcccctttccgaagccccaaatctccaagagtttgaacatccccccctccccccagcttccttgtttgagaactctgattgcacaagcagctaggtaggtgtggcgtgattggtggagggccgagggagcttgatgagctgtgatggcccctgctgcctcgctcaggactgtggacgcgctcggcgtgctgaccggagcacagctcttctcgctgcaaaaggaagagttgcgggcggtgtgccccgaggaaggggcgcgggtgtacagccaagtcaccgtgcagcgcgcgctgctggaggtgagcgaatccttggggccggacaaggcgacggagggtagggtggggatgggggacctggggggagggggtcgtccagggttcacatactaagatcttgatttctaccccgctctgcaggacagagaaaaagtgtcggagctggagccgtgatggagaagcaaaagaaaaaagtggaaggcgagaccaaaacagaagttatttgatccttcctgactcggtcacaaaacgtgatggcatggcggggctcccagcgccccctaggacaacagtcgccagactcctccccgtgaccggggacagtagatgtcccgaaggatcgcccaccctcatctcccggctcactcgctcgctcgctctcctggcgggcaggctgcgctgacagtgccggctggaatccttccgggggacctcagactgacggggacggggacggggacggggacggggacggagcatacagacactaccagagaggcacgcccaagaggcgcacggagggagggccctgggcgtcgtgacgtgctataaacagcctcctttctagaccatgcgtgtcacctgctgtccccttctctcgccggctacccaggagccaggaatctgagagatgccccacgcttcctccccataaacctggagagtccagcccaggcttcctaatcaccagtctatcctcgcactggccccatctacatcccttctcctgttcaaaaccctcgcctggctggctcctcgttgttctcagtcctgtctcctggtgtttaaggcctgggcttttctcattgtctccgcccaccctgcatttcggcccagccgctccagaccacaagcggtttgcacttaacgcttctgagggttggagcggcccccatcaccctggctcggctctcctagccacaccgtggacacccgtgtccagcctctaaggaccggccatgcagatctggacgctcccggggcatgccacgggctcttggttcttcctggcccctcaacaactttctccctgccaagccctgcaacttgtccaggttatgcaggtggatggtaagagccggttttctcatccgcgctaggtttatctaaggcctttcttttccctgcatccttggaacactcccaagagtcccaccgttgcagtcggcctctgctccccgcgcagctcagtccttacctgggccaccaggtggcgcacctcgaatctgacccaggagggccagccttgggctgacttcactaagccccctttccttctggaacactgtagcgttccagtaagcctttagtgtccattcccttggtttctcctggtacatgagataaaacctaactccagcatgacagccgatggcctgtgacccctatgggctcaggtcgcccttcctctctgttcgggactccaggcactggtccatgctgttggttctgttgggatgtcttggctccatggtgtcttatcactgcctggggcgtcatttcttatgtcgcgcttggttggtttgttggaggccgtctgggtacagccccaaactctcggtcctccagtttcagtttcctgcatgtggggatattggcaggcgccctgctgccaccctcttttctaatcgagaaaccaaaagtacaagcagttgcccaagctgttttgattccggcagtgaggtcccagactacagactgaaatgccagcaggagccatctggcttgctgggacatcaggtgatcaggtgcctgtggctggctctctgtggtttggagtctgaccttttcatcctgacttgaccctctgtcgatcactttgtccatccatcactccccaagtctacatccagccaggggcacctgtcagagctcaagccggatggtaacctggtggtcaggcctcccagctcaggtggagctcaagttcttaacagagccatgatcacacacaaagccatcacctcagcgccacagcacgccaggcctgctctaccccacgctgcacacggttctcatcatcatgcaaaaggtgcttccttcagatacagggctcaccgtcaccttctagcatctgtctgtgcagcttgtcatggggcctacttttgactgtcataaacaccacacacgcacatatatatacacaccagatacacacacaccacacacatgcccaatacactgtgcatgcgcacacacaaacacacacacatacctcatacaccatacaccctataacccacaccagccataccacacaccacatatacacagttcacctcagacagcatggcacaccacacacacacacacacacacacacacgcgcgcgcgcgcacacacacacacacacacacacacacactccgcactctccccttctccacagcactgtagctgaaatccacacagtggcaaccttcctcagtgtactggctgctggaccaagctgttcactcctgtgacgccagctggcagaacagcccattcctgactgtcaggatggaggaggcaccacgcgatccatctcaagactgattcctggctctgccccagtcactgtggccacgaaggactacttaccatcacctactcctttcccagaaaacctagacttgcggtttcctatgttggccatcctaccttttcaatgttaagccactgactccgctcacttccaaagcactgagggtcaatgtgagcacccggatcaggtcacaggcttccttctgaccccccctacctcacctggggctctttctctccagctgctcactcgagcaagctcccctccccacacctgtgagcaagctcccagccacccactggccctcatccaaatggatgagcggtttcagtcagatacacaggctgagtatacaagcaggaaccagtgccccacacccagggggagacaagtcactgagtggcaatgtcacgactttatttgtggtgcctgtgctttgtctcaaaaataccttctccccctccccagacaatgggtgggaaggaggcagcaaaaatagaagacaaccctccctattgcacacggaccctatatacaggcccacctggcagaggccagtggggctcttggcacattcctggatccctgctggggagggaagggatactgggtagcatcacacgtgaggtgggcccggggcagccactctgctcctggatactgatcctggcttccttggtccttgcttccttcctggtcccatctctggtgcctgcccactctcggcaacatttccctacctggctcagcctcccacctccaccctggttctggggactctgtgctttcctccgggttctgaggtcccgagaggaggttatggcttctcaacaacttcccccggagccctgtcactcatgttcactcgggggaaggggtgcgtgtgtcaaaagcagctgtataaatacggtgcgggagcccctccagagtcacttggagagcttgctaatgacgcggatcagtgctgcattctcatccttgagccgctggttgtcagcgcggaggtcggacagggcctagggggcagggtggagtcagctgggcagggcggggcagggtgggctctggccaccgcccttcacaagctcgttaccttcagctcctcctccagctctgcggccttgcgctccagggccctgcgctcctgcaggaatgggctgggctcagaagcagggtaagggcaggggacagggcaagggcgggacaccaccccagcggcccaaactcacgaatctctccagctccaggagggcgggcctctcggcaaagcgttcctgccgctggtaagggcagagaagactgggcgtcaggagctgcttcttacccctaggacatcagagccctgccccccccccccgagtgggggacctccaacctcccagccacggccaggccccttgccactggggctctgactcccactgccccaacagctggttcttaggtctcagtatctgcacctgcgtggcccgctcaagctccaccttgagctgtgccagccgcagggtggtctctgtcagggcctcacgaagccgctcgttctccctccgaagctccatgtacagctagggacacagaggaagcaggcaggctcagaagggcccgggaaggggccaggacagggtggggtggggcaggaggtagcatgcggcaccttccggaagcttccatcgggttcttcctgttcctgcttggattctggattgaggtctctctgcaaacgctgtctacgggcagtggagccgccatccacggtgctggacagaaattcaggccttagggcccaggccctgcccgaggggtgccccagcccccacgcatgacccggcctacctgcactccaggctccgttctgccggccccgcctcctccccctgcagaagagccctgagagttcagtctccatgcaacgtcctccctccagcccgcccggcctccacacagcatcctcacctccgcggcccctccctccagcccgcccggcctccacacagcatcctcacctccgcgggcccctccctccagcccgcccggcctccacacagcatcctcacctccgcggcccctccctccagcccgcccggcctccacacagcatcctcacctccgcggcccctccctccagcccgcccggcctccacacagcatcctcacctccgcggccccctccctccagcccgcccggcctccacacagcatcctcaccctccgctgcccctccctccagcccgcccccgcctccacacagcatcctcacctccgcggccctccctccagcccgcccggcctccacacagcatcctcacctccgcgggccctccctccagcccgcccggcctccacacagcagcatcctcacctccgcgggccctccctccagcccgcccggcctccacacagcatcctcacctccgcggcccctccctccagcccgcccggcctccacacagcatcctcacctccgcggcccctccctccagcccgcccccgcctccacacagcatcctcacctccgcggcccctccctccagcccgcccggcctccacacagcatcctcacctccgcggcccctccctccagcccgccccgcctccacacagcatcctgacctccgcggccctccctccagcccgcccggcctccacacagcatcctcacctccgcgggccctccctccagcccccccccccccgcctccacacagcagcatcctcacctccgcggcccctccctccagcccgcccccggcctccacacagcagcatcctcacctccgcggccccctccctccagctccgcccggcctccacacagcatcctcacctcctcggcccctccctccagccgccccccccccgcctccacacagcatcctcacctccgcggccctccctccagcccgcccggcctccacacagcagcatcctcacctccgcgggcctccctccagcccgcccggcctccacacagcatcctcacctccgcggcccctccctccagcccgccccggccctccacacagcatcctcacctccgcggcccctccctccccgcccggcctccacacagcatcctcacctccgcgggccctccctccagcccgcccggcctccacacagcatcctcacctccgcggcccctccctccagcccggcccccccccccccaccccccgcctccacacagcatcctcacctccgcgggccctccctccagcccgcccgcctccacacagcatcctcacctccgcgggccctccctccagcccgccccgcctccacacagcagcatctcacctccgcggcccctccctccagcccgccccggcctccacacagcagcatcctcacctccgcggcccctccctccagcccgccccgcctccacacagcagcatcctcacctctgcggcccctccctccagcccgcccggcctccacacagcatcctcacctccgcggcccctccctccagcccgccccggcctccacacagcatcctcacctctgcggcccctccctccagcccgcccggcctccacacagcatcctcacctccgcgggcccccttcgctcgtggccgacctttcgatgctccctggccgcctgtggtccctggccctgcccgtcgggcgcctctgctggggaaccagtgggaatcagctcagacaccaccataggggcccctgtctactgtgcagggaacctgacttagcccccagtgaacaaagacactttatggggagacaggatggctccctggggagcgacttcccagaaagccgacctcacctctctgggcagggccctcagcgttctccacgccagggacccgaggtctccgagaagggtcctgccggggaggagcacagtcagaaacagggagacgggtccacccgccccagttcacctgaccctgctcaccaggctgggcagggcaggctgctctggctctggagccttccctgccaccttctctgcttccttcaagtctgtcaaggtcacgccctagtgtagagaactcggtcaaggaaaaagggcctagacttccacaggactcaagttcaagaccccggccctcctccctcagacccgggagcacagccccagccccatcccacacctgtgtagacctccgagactggcgcataaggcgggagcgagccttccgctgagactcggactcttcatcacgcacaggcatctggtaggacctgagtggagaatgtccccttgggtgctgtcgcactgtgaatgacaccttaggggagtgcacattctggcagagaacgtgtcaactgggcaaacaggaccccaggagcctacccagagccccagagacccctaaacactgtcttcccctagcctctttacctccgccgatccctggagtcaggtagggctgctgcagaggctgcgagggcatttggcttcactggggatccaggctccctcctggaggatggggcggagtgatccgaagaaggaggcatggcagcctcacacctgtatggattcattcattcatcagcaaatattcctcaagcccgcattctgtgtcaggcataggagagaccacagagaaggagccaatcatggctgctgatgagccatttctgggcaaaacagataaaacaaacagcagccaaagagaccagtgtggagcttggggagaaaaggtgcttggaaaaaataaagagaataagcaattatttgatgcaccctaagggctttctcagatctcaaatgccaggatggcaccagacctgtccccttgccccagccactggtacttacagggtagagggctctggcactttctgggcaggggtaggggttattctggcaagacggggttccctggcctgtggagacaggagagaagcaaaggaggcactgtctgccccaaggcaggagcctgtaccccacacacttcacggcacctacctgagaggaggccttttctaggagggaggaggaggctgagcgctgcagaccgagaaccccctctgcacccctcctctctgagggacccagggcaccagagcttcctgtcttctggagaccgccgcgcctggagaagggagcctcttctggcggctgggagaggaagaaggtcttcattactgagcaaagcaatgacccttctcctcagagcctacgcgtgtaactccaggggaattacagtaaaccacagccaaagcaatgaccctcctcctcagagcctacgcgtgtaactccaggagaatcacagtaaaccacagccaaagcaatggcccttctcctcagagcctacgcgtgtagctccaggggaatcacagtaaaccacagccaaagcaatgacccttctcctcagagcctacgcgtgtagctccaggggaatcacagtaaaccacagccaaagcaatggcccttctcctcagagcctacgcgtgtagctccaggggaattacagtaaaccacagccaaagcaatgacccttctcctcagagcctacgcgtgtaactccaggagaatcacagtaaaccacagccaaagcaatggcccttctcctcagagcctacgcgtgtaactccaggagaatcacagtaaaccacagccaaagcaatggcccttctcctcagagcctacgcgtgtaactccaggagaatcacagtaaaccacagccaaagcaatggcccttctcctcagagcctacgcgtgtagctccaggggaattacagtaaaccacagccaaagcaatgacccttctcctcagagcctacgcgtgtaactccaggagaatcacagtaaaccacagccaaagcaatgacccttctcttcagagcctaagagtgtaactccaggagaatcacagtaaaccacagcccaggcaggtgccaccaaaaaaaaaaaaaaaaaaaacattacttcttggtccacaaggacctaagaaccaagtcaaaaagccactttcctcagcggaagcagaagtatttaccgtatcccacccgctgccccaaacctcacatctgctcagggcgctcaggctcaccacagggctcttggggctggaggacacaggagaagacacgccattgagggctcttggttgcacaggagggtgatctgtgtgcaggaacaggagaggggggtcacaggagaggccggccgcctctgagattggggacccacaagtccagctccttcctcagacccagggtccagcatccctaccagctgcctcttcttctccctcatcctcatccccaagagaggggcccgcggccccaccaggccggcgctccttcgacagatcctgcagggagatcttctcacggctgctcaaacgacacacggagctcctaggaggacagggtgtccgtgtccaagtctgggggcgagtccgacccaccccaggcctaggcatctcttaccttctgtgcttgctgttggagggcacctgtggctcttggcctcggctctgagaggcttccttttggttccgaagctacaaggatggaagggggcaactggggaggggcagagagcacaagccctccagggtctcctggccgccccctctgtgccacctctccacctcgagggccatcacgcataactgggctagtcacactttatgcagggtcctgcaaacatgggggactcagtaacccggcagcacactggctctggggcttattcaggctctcccaggcttggcctggtccagctgtcactgcctccagcctcattcccagggggattcgtcttcttcccaggagcgagcaccttgctcagacttccccctaccctccagcacatccagggcaggacagggcaggtggctctttctggttatcacaggccagctctcagctcaaggacaacggccaccgtcccatactaagcagtctggtgtcgtaaccccaggaacacctcttgcccatgccctccttgcatcccagtgtgccacgggactcctctctggacaatgttcccgatggttccacgaggcccgggccacctcactaaataatggaattgcagccatgccgtctgcttggggccacacccatgatgcctcactctccactttcctagcaaaagtgctaactagagtggggggggggtagatacaggttcaacctgtgtcacacacagctgtcttcccaagcgagcaggcaggaaactctgggcatagcctcaagtcctccagatatggaggtgcctctgttcttagccctccaccagagctgggctgacaggtgggaatagcgggtctcagtactgagggtgtcaagggacaaagactgtcagccctcccggttactgttacctcctcagagctgccaagtaaagaggcaaactagagtcgagactcacgtcctcctgtttctgggccagttcctccaaaaggttcatcacttcctcatcagccaggtcacagggccgctgcccctgagtaggagaaggaggcagatgacggtgatggtggtggtgtagtaggggctcccccgccaccctgccccaccatctgagatggcccttaccgcatgggtcagcgaatccatgcccccaccgtgctcagccaggagacggcaggcgtcctccacaccccagtgggctgctgcgtgcaacggtgtccagccatctccatcccggagctctgtgtcgtagccagcttggagtagcagcctaagggccagggaggcttgggtcagatggcaagctaggccaatggctgatctcaacttctgttctgtggccacaggactactgatcaatacccaagcgttactagttttaccagcaaccagccccaccccaagctcaactgagccctcccttggaccagcagctactaatgaaaaagctccctcataccacagggatcccactcctcaggccccagggtaaagggttagggcagtggtgaggcgatgaggtggatgcaggactccccactaacgcaagcccatggagaggatggaccctgaaggggctgtgatgctggaaccactggaaccacgcggttttaggacacggatcctcaacagtgtcaagcagctctcacaccctctctacaactggagacatcaccactagaatcctaacttacgggtacaagcaggaagcaccagtgtgtgggagctggagaggctgctcaaccccctcccacgcacaggacagccctaccacagcacggtaagaccccaaacatcacagtgccggaggagagcgagcctggctcagccttccagaaggtaacaacctggagctctcaaaactcagcatggcacgaggcgaggcctcttttggaagcagtgtgatgaggtcctgtgtcagtgaggaaggcttcaagcccagggaggcagaggtacaaggcacaaggtgctgtgtggccctgggaccctcctccctcacacttcccaagattcccctgtccccttgcagcagggcacgctgggcttcttgttacattcccacatgccagggtctctagccagctgtgcgctccttctggtcagtatcctaggagcctgaagcgtgccacccagccacaccccctagtccatcagcacttcctcacctggcagtttcttcaccaccatctctgccagggggcctccctactgcccactagttatagcctcccaaggccaaggttttctttgtataagcttagtgttatttaccattagtgtgtgtgtctgtgtgtgtgtgtgtgtgtgtgtgtgtgtgtgtgtgtctgtgtgtgtgtgtgtgtctgtgtgtgtctgtgtgtgtctatgtgtgtgtgtgtctgtgtgtgtctgtgtgtgtgtgtgtctgtgtgtgtgtgtgtgtgtctatgtgtgtgtgtgtctgcgtgtctgtgtttgtgtgtgtgtgtgtgtctgtgtctgtgtgttgtgcataaatgccaacacacatgccccagtatgaagatcatggatgaagatcagaggacatattcaggattcactttctccttccaccaccggttccaggacctaacacaagtcaccaggctcttgtgtggccaacacttttacctctgagctatctcactggtctagaagccaacgtttgcagctggaccctgctactccccagaggacctgtggcaatgtctacagtcatcacacaactgggtcagaggtgctgcaatggactggacagccatcagaatagaatgacccagcccatcaagtctctcattggctacggtgggtacacatctgaaacaccacgaccagcccaggaggctagcccctaacagacaccaatatttacctgtacttcaatgagtacaatcatagaagacttttaatacagtcagaaacaatagataactataaattcagtgaacaggagtctaaacgcaaactcacacaaagggggccatcacaaaattacaaaattcagtatgatggctcacacctgcaatcccagaacacagaagctgaggcaggaggacagctgtgagtgcaaggccaacctaggctatctatccagtaccaggctagtcaggactacatagcaagaccttgtctccattagaaaagaaagaagccagaggggagggaggcaagcatggtggctctcacctctatcccacaggaaggtgaaggaacaaagagtagaaattcaagaccagtgaactagaggcgatcatgaccgacatgagctatttatggaagaggccaaataaacaaacacaaaagttgtcatcagtgcattttttttttcagggctgggactggaacccagaacgctaggcaagtgctctatccctgaggcaccccccccttccctcacgggtagacaccagggaagcatctatctacctatggcctgcgaccacagcccagtgcttcagttctgggacaagtattggctcactttctctactaactagccccccggacctatgcaggtgacaccggggaaagcatttaagcacaaagacaggaaggagttctgatcaccagaatccacttaaaaactcagtggatagctgttataaaaaaatgacatcagggtggagagagatagatggctctgctcttccagagacccgggttcaattcccagcacccacacggcagctccaggggttctgacccctcacactgacataacacagacaggcaaagcactaattaatgcacattaaaaaaaataacatcatgaaatctgcaggcaaatggatggaactggaaaaaaaaaaaaaaaaaaaaaacatcctgggtgaggtaacccagccccagaaagacaaacatggtgtgtactcatttacaagtgcacattagctgttcagtgaaggacaatcgtgctacaatccacagacccagagaggctaggtaacaaggagggctccggggagggacggtgcacggatgccccagggaaagggaaagagaaaagactttgcagatggaatgggcaggtagggatggaaacaggagaggtggggagagggagtggaggggaaatactggggggggtggctgcaatgggggctcactttgggggtgttaaggaaacccagcacagtgggaactcctggactctgcaagggtggacctagccaagtaacgagggacacagagtctgaaccggctactttgggtaacaggcaaggctcccagcagtgggacatcaacccggccacaaaacttttgacctacgatgtgccctgcctgcaaggtgtgctgaggtaatggtggcgcagagcttgtgggagtggccaaccaatgacaggtccagcttgaggtccatgccacaagagggagcccacgcctgacacagccttgatggccaggagcctggatagcccgagacctggggtagaaccaaatacaattgggggaaaagaaaaaaaggcaagaaacaattcttaatgatattctgctgttctcatggatctgtggctagcccaactgtcgtcagagagctttttccagcagttgacgggagcagatgcagagacccacagctcagggaaccccacaggaaggattatggggggggggggcgcgaggacaccaggagaacaaagcccacagaatcaactaagcagggctccttggggctcatggagactgaaggagctagccatcaggacctgtatgggtctgcgctgggtcctcgcctggtgctcttgcgggactccttaacactgggactggagctgtcgctgactcttgtgcctgtttggggacccagacaagcataactggttacgctgtgcttggctgtcatctctgagatgcctgttcttttctgaagggaaacagaggactggatctggaggaggggtggaggggaacagggcagaggggagggaggaatgtaatatgagaggaaaaaacaacaactacaattattgagtggacatggcagcccatctgcagagacaggccaccctcagacggagatggcagctaaacttgccaaaaaggcaagctgagggatcggccagaggccctgcctcaatattagagtggagagcaaccagagaaagtactacatgccaacacacacacgagtgtgaacacacacacacacacaagtcatacccatacacatgcacacgcgcgcgcgcacacacacacacacaccacaaccgttaaccagacatatagttgtgtggaaacaaacctagttttccttgcaactaggactggccaatggtgagaactgggttaatggaacacagatattaaatatgcacacttctggaatgttctcctgaaaaggaatagacattcgctccctttgcctctgcttcccaccaacttgagatatagacgcaaaggcaggtgaggcaagtcaccctcaagtgagaggcaccgctagagcagggcgcaagctctgcactcggagatttagggcatcctgtcccccaaaaggaatgggctcagagcgcactgggactcatgctgtaactacagagactgatgcccctcccccaggagcacaactatgcaggcaggctgtaagtctgggggtggcacgaggtcttaaatcctgctggagaaaacctgcctgcaaccttaccagtatgaaaagcagagaggttcatcttaattcaatttgggtctttgtttttttgttgtttttttttacaacaggatccctctataaagcactagcctcacactcagtatatagacaaatctatcctggaattccagtaatcctcctgcctctgattctcaagtgtaattatagacatataacaccgtatcaagcaagcaagtgcacacacgcacgcacacgctcttgttacatagcctgggctagcctacaactcacagcaatcctgcctcgacctcccaagtgaggaaattaaaagcgtataccaccatgcctggcttaatgccatttttttaggttggtattatttttatgcgtatatgttttgcctacatgtatgtatgcatacaaatacacacagacacagagataaataaatgtaatttttaaacctctttggctttaggtatgtaaaccaggagaagaaaaggacaagagccccgaaaagcttccagacacaaaacaatcactctggcctcgctcacctcatcacctcgatgtagcccttggcggcagccacatgcagggcagaggccccggtccgggggtggcgggcctctggcatggcacccccattcagccagcaccttgtgtcatgaagcagcagttcttcttcagcccgcttggctgcctcgacatccacacctgggagaatgagaggtgacaggtggactcacacagggtggcctaggaaaccccggctgcggtctcaactagtcacagcccggccccgtgactcatcaagtctctggaccactcaggagaccgggactgccccagtgtttcccaactgtgctccctgaagacctgggcaccaccgagggggccaagacaggccaggaatggaaaccacaggtcctgacccctgtgggtcagtatcctctttatgtttttctaatagaaaaccccacaccggattccatctaggttttcctacccctccagctataagctaaagccagcgccttcacacaatgtcactgctggttcttctccctttgaagtacgataggccaaacaaaacttcactacggcgttgtacgtggtggctccggcctctattccagatctcagccttggcaggatgaccggtggcctcgaatctgagaacagcctgagctacatacatggtgtcaagccaaccaggactagagagacagactctgtcttagacaacagtaaaaactaaaactcaaaagcttctgggcggtggtgcacaccattaatcccagcactcgggaggcagaggcaggcggatctctgtgagttcgagaccagcctggtctccagagtgagtgccaggataggctccaaagctacacagagaaaccctgtctcgagaaaaaaaaaaaaaaaaaaaaaaaagctatttcccaaactatttgcatgcatagtttcattcttgcccagatgtccaggcatttgacacctcgctggcccacgacagaagtgagaagtgagtgactgccttggcactttgtgcttatgcgggtatgctgcatgcctgtgaccccaacacaggcaagaggcaagagaccagcagggctccacagagaccctgagtcaaaagacaaacagagggggaggggctggagagatggtttagaggatgaagtgccaagcccgatgacccaagttcaatcctgggaactcatgaggcaaaggaaagaatcaagttgcacaaggggtttccctttgtgaacccagcttggcctcaaactcacagcaatcctcagtctctggaaagctgagattaagagggggtttttttgttgtttatttgttttttttggttttggttttacgagacagggtttctctgtgtatctttggagcctatcctggcactcgctctggagaccaggctggtctccaactcacagagatccacctgcctctgcctcccgagtgctgggattaaaggcgtgcaccaccaacacctggctaagattaagagttacacaccacacctgtactccctgcactcaaagaggctgaggcaggaggattgctccaagtccaaggtcagcctgggcgccagcatgagagcctgtctcaaacacctcaggggggaaacagaaagccaggcagactagctgaggctgaagcactccagccctcactgtgaccctcatcccttaaagcacccctaactcactgagaccacagcaaaatggcctctgctgaataacttcctcctgggaaggttattactgcccatgcttttgcagttgtgaaactcttgacttgccgaagttcctcagagttgagctgttgtatccagtagccggcagctatgtggaactgccgagcgagcactcgaaactgagacatgctgtgaacgtcaagtgcactcgggatttcaaaacacaggaaaaggtaagggctctcgtggacagttgtctctaaactgttttgtgcacacgtgcatggcagcgtggtcagggggcaattctgaggagccgagtcctgcttcccaccttgctgaggctgggtctctggattctgcggctgtgctgtgtactctaggctacccggcccacagggcgtccccacagttctcccaccttcctctaggacctgggagtacggacgtgcaccagtgccgccgccggctttttacaagggttctagaggtggtaacttgggtgcatctaacatctttactggctgagctatctccccagttccccttactgtgttgattgcacccaacggaatactgggtttgttttttgttctggagcgtgcgtgagagagagagagagagagagagagagagagagagagagagagagagagagagagagagagagagatacagacacagaatcttaacagagagacagagtcttacattatatagtggaggctgaatatctagtcttcctgcctcccaagtactatgggatagtccttctgtacgctacgaatatggactcttctcattggttaataataaagctgagttggcccacagccaggcagaataagggtaggcgggaaagccaaacagagatacagggagaaagaagggcagagttgagtgagacgtaagcagccaccagggaagcaagatgccaggtgacaggtaaagccacgagccatgtggcaaaacacaggctaatagaaatgggttgatttaagttgtaagagctagttagtaataagcctgagctataagccgagcattccgtaatcaatacgagctcttgtgtatttatttggggcctggcgattggaactaaagggaagcttagactacggacttgcacctccatgcttagtttatgggttcagaggaccatgctaatggataagcactctaccaactaagctacacccccagcctatggcttgcaagtttcaaactacatctgtggctcattcaggatttccactgggcgtcactggcaaaggccttcaggtcccacctggagcgctggctcagccattagagccattaatggtagactcacaacctacacaagagacaaaaacccacacaaggggtggaatgcagagactcaaccagttccaagccagccgggactaacaaagcaagatcctggctcataaacccaggagcagggtttagcccagtggtggtatgcctgcctggaaagggatggccccaggttcaggcctctacacagagggctgctttcctcaccacactccctcttaaccaaggtgagcagccgctcccctcagcacacacattgtacactgccaccataaagctttacatgggacccaagaaacagtcctgaaagctggttcgggatgttctttctcattgcaaggcaaggccaactccatgcggacaccggctgcagcttggtgctacctggcggcagccgggtcctagctccttgtgtctcctggccaactagggtttcccttgtggtggcagagttcaagaatgcatggcgaaagtccacccgcagcacagtcacagggaacagggcagggagggccaggcccgccctcgtcctccagactcctgcttccttaaagggagtctcccacagttccacctactgtggggggaaggggaaggggaagggcggagctcccttgctgttcttcaaccaccagccagtactcccgtgcaggctcaagggcagcctgtgctctccacacagccaagacctgcttgcttgttactcagtttttcttacacaggccgttagctgattaattgggtttttattttatgtgtatggatgtgttgcctgtgtgcatgaatgtatacatgtgtgctggtgcccgaaaaggccagaagagggtgtcagattcctctggaaatggagttacaggtgtcatgtgggtgctagagttgaacccgagtcttaaccaccgaggcatagccactgattcagccagactgaccagcctgcaaaccccagggatcctctgtatcggccttgccccacacctgctaggattacaggtggtggtgggcttggctttgtggttgctggggaactgaacttaagacctcagtatgtgcaccaaacccttctactgacttagcaacattcccgtggaagtcctgaaaatgaaggacggggggaaggatactgactctgctgtaagaaaattcttacatttatgttattgtgtggatgtgtgtgcactcaagcacaaatgagagctagagagggcctgcagaagtcacttctctcctcccaccaagcggatcccagggactgagcccaggggtcaggcttggtggtgagcgccttcgcccactgaagcacctcaccagcctgaaaataaagctctcatggcacccagccacgtctgctttttctcatcccgtcgctggctcatttcccccgatagcagctggtagagtcattatagcaagaccgtgcaccctgtaaggcctgaaacacatactgaccagccctccacaggtcccagctgactcctgctgggaccactgagttataaatcagagcgtcatctaccggctgcagaggcgacagctttttggtgtcaccaacagcaaacactgtgctgtattcctgtgcactcaccacctgtgagaaaatgcaccagggcaggagctcaggcctgcagcttcagccaggttaaggccggcctgagctctccttccaggcacagccttgcactactctagctggcatctgtaactaccgcagtccactgtgcccatctctgcatgctacagccctcactgtccttcctggatgtcagtttccatgggagacagcttcgcctttctccaaagcacatcctaagtctccttcctaccctgtccccccaaggggggctcctcctccacggacactgtgctttcagtctttgccaggggtctgacctaggcctgggccgcaccaacactgctaggacctggcagcacccactcttcctccttcaggaaccacgtcctgactttcctcgcccacagggcttcagtgtgacacttgtcacaaggtgacaagtcctcctgcacactggtgggctctgggactgacatgagatcatgtgccagtgtcacacagagagctgtggctcagccactgaggggggtagggctactgtaccccacctaggcattagccctgctaagcaccacagggagagaccagaccccaccaggaggccaaagcggctggtggcattagggatccactgccaaagactggaactctagggcttggacagacagatggctcagttggtaaagtgttcaccacacaagctgcaaacctgagttcaacccccagcacccatgtaaaatgccaggcatggtggagcatgtgtaatcccagtactggggaggtagagactggaggacaaccggggttcactggccagccaaactagcccaatcgtggaagctactgagacaccctgactcaaaaatcaaggtagacggctcctgacgaacattcgattgacctctggtctccaaacacacctgtgcacgcacacatgcacacacaaacatatgaaggactgagattctgcatacgttaaacagcaactctctcctccccctttttattgcattcatttactggtgtgtgtggctgagaacaatctatggggtcagttctctggtccaccaagtgggtccaaggaatcagactcagttgtaggcttggcagcaagcaccttgacccactgagccatccagccaactctcttttttgtttgtttagtttgttgggtcagaatctctctgtgtagtcgtggctgtcctggaagccactctgtagaccaggctggcctcaaactcagagattcacctgtctctgcctccagtgctgggaataaggcatgtgatagggttaaacccaccacacccagctctactcccagggttgttagcgcctggaaaccacattggatgctctgtggctatgactttgggagcgctattattttcattacgcttggtgtctcacacaaatggaaacctcgcacttgtctttgagtggctgacttgctccatgtaggtccttaagtctcatccaaatctggcaggcggtggtggcgcacgcctttagtcccagtactcaggaggcagaggcaggcggagttcgaagccagcctggtctacagagacagttccaggacaggctccaaagcaatacagagaaaccctgcctcaaaaaaccaaaaaaaaaaaaaaaaaaaaaaaaaaaaaaaaaaaaaaaaagaaagaaaaagtttcatccacatcgttagacgtgttggtttccttccctgttaaggatgagcagcattcttttatctgtatagaccacattttgcttatccagccagtgatggacttcctaccctccctggtcctgaggctggaattctcataccagaccaagtgccacaggctggagtactaagcaacacccaagtcctaggctactgtaaatacgcctgcaggtggctttctctttgcccaacaccacaaaacataaagaagggagccagacatagaggcaactactacaatccagaccctcaagaggctgaagcagaaggatccaaaaccgtactacagtcagtttcgccgcagccgaggccaactaactaattaattaacaaaataatacgtattgtgggtgtgcatcttgcgatgcttgtgttgtggtgtggggattgaacctgcctacgctcagcaaacgctctgccagtatgaagtgtccagtcctctcccacacacacgcgaacacacgcgaacacacgggaacacacgcacacacacacacacacacacacacacacacacacacacacacgcacacgtatatttaagatctttcctctctctctctctctctctctcacacacacacacacacacagggttagttaagaccttatttgtattacttttaattgtgtgtgcgtgtgtctgtgcagggcacgcacacacgtgtttcagtaccggaagaggtgtgggatcccccaggtgctggagctacagtgagccagacactggtgctctgaactgaactgaatcctctgccaaagcagaaagcactctctggactcctgcttttgttggttttgttttgttttgttttgttttgttttgttttctttgtacttttcaacagattctcactaaactgtccaagttggcttgaactccctctgtagttcaggcaggccttgaactcacaattcctcaggcctgggagagcggaatcttttcgtcaagaaaacaccccatttgaaaactgaggaatgctgtatcagcacaggaagggggaagctcaggccttgtggcttagagaagcggccttgtgccatggggttaagagccccaggctgccccatctcgttggctgagggaggcgcttcccgttatctgagcagagcttccagcatcagcaacatagtctccaagtggctgaggaatggaaggaggatggactggaaaggagaaaacaggaagggtactgccgcctgtgtgtggggaaaggggcagagctggacaaaacagtaaaggcgtctatttaaagtgtgcgattccatctgcacgaaatgtccgcaacagacagatccctaatgagagaaaccttagaggcttgcccagggatgggacaggggactagacttttgagggtgactttaaaatgctctgaaatcaatgtggcatcgactccgacaactctgtggaccacagcacaaccaggaagtgcactttggatgggcaaacttctgggcatattaattacagccccaaaggctgctttgttataaaaagcactggtgggctggccggctagctcagcaggtcaggcgcttgccaccaacaacctgagtcccagaccaaggccacatggtggaaggagaggcctgccagaaattgccctctgacccccacagtgccgagcactcacactcatgataaactaaataaatctaaaaaacaaaacaagacttcaaaagcagcagatggagcgctgacacagacactcgggaggcttgggcaggacggctccaaattcaaggccagcctgatctacgcagtgagtaccaagccagccaggacttcgtagcaagaccctgtctcaaagacataaacagggctaaagggctgcctcagtggttaacagcgctggatgctcttcctgaagacccaggttcaattcccagcaccccgggcaggcagctcacaaccatctgtaactacactcccagggatccagtgccatcttctagcctccgcagacaccaggcacacatggaacaaaataccgggacataaagaacacactgtgtggtgaagcccagggaaggatctgtggtggctgtcacagtaccgaggcgactcttctgagtttgaatcaggggacgggaaggagagctcagctcaaccgctgctacctgtggctcctgaccactgcccttcagctcttggtgcccactggctaccaagcattcccaagtgactcgcagtcacctgaaattcaatatgccaacatggtgaacccactgtctctccatcctgcgtagcaacacgcaaggacggggagccaagactatgcctcccatgaactatctgtcctctgtccccgcttatctcctaactggacagtccccagtctggaactggtgccttatgttcctggagagcctgcaaagctgcctgtttgctgatccctttccttccagaccctgcactacagagctgagagccacccagctataacccagtgtttcgtttgtagctgacagggactcacagagcccaggctgctcacaaacttactatgtggggaagcctgacctaaactcctgatcttcctgccctgcctcccaaggctggctgggattacaggcctgtgccgggacacctggccgggacactagcttgtcaggcaggcagagagggctctcaagccctgttaagaacttgctattgggaacacacacgccccacccaggaaaatgaataggacccaacatggagtttcaaggggcatgatgggagctcaggagagaatcctctgcatgctccagtgcctcctaacacgagctgggtctagccatcttgctgcttactcctcgacaggcccttgctgacagcacctccctccttcagttcctcagacactcacagcagttggggctcttactctgtgtctggcagtgtctcactagacccttggcaacccaccctggggacacgtaccacccccacttcacagggaaggaaactgaggcacaaagagcaagagtacaaggaaatgggctgggcctttgagcccagactcccagacgccaaagctctcgatcccacaggcccacctcggcgggcgatctccgccttcagcagcccctccatggcatccgactcagccaggtccaaagacaggtctccatcactgttgacggcggcgatgttggccccatggctcaggaggtacctaggggcaggggaaggtcagagccaccaggcctggacctaacgcctaacccaagccctgcccttcaaccccagcctcacctggcaatgtccaagtacccacaggaggctgccacatgcagcggcgtccagccctcgttgtccgcctggttcacagtagcaccctgctccaccaggaagcgcaccacctccaggttctcgtctatgcaggcctggggacggggacaggcccatcagctcccggccgggccaatgagaggtgtggaaagcaacgccgatgggctgcagcacagattccaggggccctctggtcagtggccgcctaaaatatgcctcgttacccatgcttgggtaatctatgcatgcagagctcatggagactagagcaggctccaaaaggcagattgaaaaggcgaccagggaagaggcggagctgccatccctgcatgtgactgctgaacataccctatgaggcagaggaaccccagagcccagccatgttcttccaaggggcagggcaaggctaggttgaggcaaaacgctcacctagccctgggttccatccacaacacaggaaaaagagagatcaccacaaagagacacacgcacatcccagagttgagggcttggggcacagttcccaaaagggatgagtaggctatgttcccggtgtccagggatccaagcagaccaagctctgggtcactgagggcctaccgtgcacaggtctcctcagaacttcttttctaaacaccccacaccacactaatcccccacctcctcacccttcgaaccaacagctcaggagaggaaggcctcaccctagccagcacagcacccagggccacaagagagctggtctcaatccctgatgacactgcttggacactggaaccactgagtctagggagggggttaggtccggactcctgggtcctcatgaaaattaacccccttctacaagcgcgaccatctggagaaagagggaaggaagctacgagggccaagtgcatgaagtcatggaaatttaggctgggggggggggcacgtgccctgggaacgggatgaactctgggcttcactctgggctcagtttatttccaccctgttgtcatggtgatgggagggggggcaaggaggcagatgggcctttccctttcaaggacctggccgggtacgggcatccatgtgaaagatgcctgaggctgggcactggggacccaagaatcctcctccctcagatgtagaactctagccaatcctctttccttagacccagggatccagacttggccctcctccctcaggcccaggtgctagggctccccatctctcccctgctcaaacctaggactcttaactcccagccctacctactccagacccaactcatagccattattggacaaggcaattattggacaagggaaagaggaaggaatgtccctgccttgctaaggcagaggctggggcttaggaaatgtcattgcaggaggctgatgccccaaggagggtctagaaccggaaacactaaaaagtctgaggtgtagaaatcaccacagactgggtggctcaatgcccctgctttcctgggactgaaactagtttcaggagttttcactgctgaagccagggcagtggtactaggaggtgatgctacgtacgcaccactccaaaccccagccccctctgcgttctggccctgaaagccaaatgatctcactgaatctgatctccagtctcccaagcctcctgcaaaggcctgaagagtcaggtcaccaaggtgtctgcatggcgggaggagagtcccacctggaaggctgacacgtcaggcctgaggtcacaggttcctgtcagagaggatgctctagggacctccagcagatgcagaggaaggggatgcagttgggagggaactcttgggagggccagggactttggtgatcatgtgagcagcctgagctgatctcctggactggtcaaagacgctgacaccctgagtgtggcctcgggaaaacaggaccctgctatatatagaggacgatgtcccacacagctcacgccggccccataaaggaagttttccacaggacgcctctcaccatagagtccctctggggacaggggtgaccactggtcccattctacaggtaaaaaaactaaggcgcaccgagaaaagacactcaagatacaagacacaagaagcagactgacacaaaaagtcagcacggactttttttgtttggtcaagattttgcaactgggtctcatgtgggctatcaagatggcctcaaagtcactgtgtagatgaaggtgacactgaattccaaccctcctgcctctactttccaagcacctgatttctgtggtattggggttggaacctgaggcttcctgcactctaggcaagcactctgtctaaaagacagcccagcccaggacagacggattctgtttttcctctgcctggatgagtgaaacactgaacctttattccccacctccacgagcatcctagcaagaggacgacaacccaggagatggaagttgccatgaaagactgaaagtgaaccaacactgtggccaggaggaagaagaacggggatgggggctctgctgtgactaatcttgtccctgacaatgccagcttttggatgacggggagataaaagcatcccgaatccagaaggatcccggcaatacaagatggtcctcactctcggggcacagatcactggaaaaagataatcacagtgtctgagtcgcccagggtcctggtggggtaggttctagaaggtgacagggtggaaatctaagagacagggcatagtttttaaagcaggatgctgcccaaatatagtccatggggtggtaggtggagtgggcatgcctgtaatcccacaaatgagggggtcagagacacaggacctgtatttgaggttggcctgggctacacaggggaaaaaacaaaaaacaacaacaaaaacaaaaccagaggagagagaaatagggcttggaagaccgaatgcttagaagtttccagaaaggcaaatcaatgtggacacagagagagaaagacagagagagagagagagagagagagagagagagagagagagagagagagagagagttgaggagaaacctatggggggtgggggtgggtggcaagatcaccaaaaggggatgagccgagaattgaattaataggacatggggaggggaggaaggttatgggatggggcccaacagacggtggagcgcctcttctccaggggaacaaaggggtacactgccttggaggggcaaaggaccctcctgaggccacagcggacagcacgggtcacaggaagtggggtagggaacaaggtggaccccccaaaagaagtgacactgaagggcctgggcctggctagcctcagaggagggagtgggggattgggggggggggcgtcaagtcagagctgggccctggaagcctgcggcacagccagggcagccaccagcctggaaaggcacggggtgtaagccatccgtgtgcggaagacgccgccggggagagcggtgacagcgcggatgacaggggcgaggcggcccctgcagggcaggaggcggggagggaggaggggtggctcggggggccccggggagggaggatgctcgggggccgctgacctggtgcagggcgctgatgccgtcggcgttggtggagtcgagcaccgctcgggcaggcggcgggacgccggcgtccgattccccgctggcgccgggatcggggcccccggggtccccgggatccccggggtcggctgcgcgaagcatcaggcgagcctcatccagatcgccgcccgcacaggccgccaggaactcggcggcgcgctcgaagcgcacggtgcgagcacgacgctccccggggcccggctcagcccccgcccgcgccccccaccgccgcagctgctcctgccgccgctcgcgggctgccgccgccgccgaagacgacgacgacgacgacgacgacgacgacgccgccccggggccgtcctcgcccgacatcgcgccccacaccgggccgctcgcccgctcacccaccgagcgagcgagcgagcgagctgagcgagcgcccgcccgaaggccggccggcgacgaacagccgccacccgcccgctcgctcgctcgcccgcccgcccgccagccccgggggccgccgggaaccgccgccgccgccgccgccgccgccacaagcaccgccccgaggctcaggctgggccccacccctccccccacggacgggcgttgacgtcacgacgctgccccacagccctctgggaaatggagtcctccgttgagaagcccgcagggttttttcagcagactcgctaactgctgagggaacggtcggggtggcacggaagccgccagcaggcgcgcctacagcccccagcacctgagaggcaaactgctctctcgagttcgaggtcagcctgggctacagaggcagtgccagggtagcaccaattgcctaaagcaggacacgcccccccccgggaatgctggaaatctgagtttagaggcgggacgggatgcccggggggatgctgggagatgtagtttttttggtaaagcggcgcaaaggatggcgcgtgggaaatgatggcgtgtagcggaacccgagagacgcagaaataagactcgcgtactttcagttgtgtttttgctgtgagatgggtttgccctcgagctcgctgtgtgactgcgattgtctgttttaaactcccgaccttcctgcctccgtctcctaattgctggggttgcagacgtttgtttgggttttgttggtttggtttgggttgggttttttcttgggggcgggggtattttgttgttttgtttttgtttttgttttgagacagcgtctcactatgtagccctgcctggcctgaaactcgctacgtagaccaggctggcctcgaactcatagagactcctccccccacacttctgcctggtatgaagggggcgccaccaggtcccgcttgttttggttttggaatctgcccctccctccctccccatcaacacccgatgaaggacaaggatttgtgaatgaatgaatgaatgcatgagtgcatgaatgaatgggctccccaagacgtcggggagaccaggggcccacgggaaactgagtcctgaaaccagattaaacaccaatcgccgccaaactcctctgggtaactaaggttcccgtgcaaaatccaagggtatcgggtagcatggggcaagctgggaaatgtagtcccagggccacgcctcctaaagagttcagcccccagacttccaaaactgcctgagatgccaaggtaccccggaaagtcagtttccagatgaagacaagcctccggtctccagcggtaatcccttgagcacccgggaagaagggtccccaaagaaccacacatttctccttagcccactcggggctgcgggggacgctaggagatgctctcccggctgcatcaatgctctcctggaattctgggatcggtagcacaaaatgtgatgctccgataggtttggaagttttgttagtagacccaacagataaaagaacaccttgatctttcaagaatcttccccccacccccacccccacccccacctccaccccaaaaattgcaatttgagaaggacagaaacacttttgagacaggaacacagactcacacacacacacacaaaaaagtagaacagaaagctgtcaagtttatagagagaaaacacgtcttcctaagggtcgttagggcagccccgttcacactgtgacccttggatttgtgaatgagagataaattacagaccctggcagagtctagggaataacgaccataaatccaaaaggataaccctgtggtttttaagatgtgagatcacacacacacacacacacacacacacacacacacacacacacacaccattcttccccaaggcaagaaatcagatatttcaacccctggggtccagaaggaaggaggtcgctgactccaaaaactgtcttctgatttccaccatggatttccacacacacacaccctatcaacacacacactaaatagacgtttataaaatgatccacaaaataaggctacaccaacacacagaggtaagactgttgttagacagttttggtctggttgggttttttttttttttttttttttttttgagtagccttctcctgtcccatttctcatgcctctacacacacctggcctctgggtgtgttattttaaaacatccttagaagaattaatgaccttgtacaaccagtttaaatgcaagaggcaattaattttgttttgttttgtttttcgagacagggtttctctgtgtagctttggagcctgtcctggcactcgttctgtagaccaggctgacctcgaactcacagagatccccctgcctctgcctcccgagtgctgggattaaaggcgagcccggcagcactggagatttaactcaaggtctcctgagtgctcgacaagctactcccagccatgaacttgatatctctttaatggcagctgatgtctctcccgggcaacatggagctgtccagccaagccgcacagccagccacgcataatgacaacacggaagaactcaagcggatgtctggagggcctttattttgagttacagatgggggacacactccagaggctcccaggctccatgcagtggggcgtgtcctggcagtctcacttccagcggcctccaactcgacccttcccagccccctttcggctgtgggagaagaaggtggagtcaggaagaagcccggagcctccgagataagcttaacacagtccctttaaaattaaggaagtccaccaaatacccacccccacccagagggaagagagagcagaggtcagcagagctgtttttttttgtttgttttttgggttttttttttgcagtagtgagcataaagtcaaggcctcacacgtgctaagtatgttctgtacactgagccacgccccttgcctctcactggcgattctaagcaagggctctaccactgagccacatccccagcccctcactgggggattccaggcaggggctctaccactgagccacgcccccagcccctcctcactggggggattctaggtaggggctccaccactgagccacacccccagcccctcctcactggggggattctaggcaggggctccaccactgagccacgcccccagcccctcctcactggggggactctaggcaggggctctaccactgagccacgcccccagcccctcctcactggggggactctaggcaggggctctaccactgagccacgcccccagcccctcactgggggattctaggcaggggctccaccactgagccacacccagcccctcactgggggattctaggcaggggctataccactgagccacacccccagcccctcactgggggattctaggcaggggctccaccactgagccacgcccccagcccctcactgggggattctaggcaggggctctaccactgagccacacccccagcccctcactgggggattctaggcaggggctccaccactgagccacgcccccattaagggcatctctttcagataattcccagtagggggttggtggccatgttggagttgactttcttgggttagttcggagaacacatgcaaatttatgagtaaggggcctgagggagaaggaagggtgagctggagttggtgacttgcatgcaacaatgttgagtgaggctggaacagtacagaaaatgctagaaaaaggcagagactgagcagtgagcggcctggatacggtggagcacatcggtaatctctgcactctgaaggggatgaggcaggaggatcaccgagagtttgaggacaacctgggctatatagcaagagcctgactcaaatgaaaacaacaacaacagcaaaaaagtcgggtatgatggctctgtaatccctgaacttgggaagcagaggcaggaaagtgtcaggagttcaaggacaccctcaactacaaatggagttcaaggtcattcacgcttacaggagaccttgtcttaaagcaagaaatagaaggaaaaggggcaggaagtggacagacagatggagaaggggggaggggggaaagaaaggaagaaagagagagagagagagagagagagagagagagagagagagagaaagaaaggcagacagaggggggcactgagatggctcagcaggtaaaggagcttgcagccaagcctaggccctgagtttcaactctgggacccacatgatagaaggagaaaaccgacttgttcgagtcatccttcggccacatctgcaccataacagcacacacacacacacacacacacacacacacacacacacgcacgcacgcacgcacgcacgcacgcgcgcgcacacacacacacacactatgcggtgtgatatgatacaaaaaaaagtgtaaaagaaaatgtactcagaaagaaagggttggagggaggcagagaggcggggagattgagaccaaagagttgataaagagaagcaagagattggagtgcaggccaataaacacagcactcagcaggctgaagccaggggaccaggaggagttcaaggtcagcctcagctacctagtgagactgggctgcatgaaaccttgccttaaaaataaatagacagagccgggcagtgatgcgcacgcctttaatcccagcacttgggaggcagaggcaggcggatctctgtgagttcgagaccagcctggtctacagagctagttccaggacagcctccaaagccacagagaaaccctgtctcgaaaaaaccaaaaaataaatgaataaataaataaataaataaatagacataccaaaaaaaaaaaaaaaaacaggaacagtgagtcatgccaatcatccccacatacatgggattaaagcaagaggatctcctacaggttcaaagaaagcctggtctacatagtgagtaccaggccagcctgggctacaaagtaagacttcctcgaaataataaacaaactaaacaaacaaacaaacaaataaataaacaaacccgagagaacagatacagaaaggatgtctcagggagcaaggaacaaagacatataagatgccaaaaggagggctggagagatggctcagcagttaagagcactggctgctgttctggaggtcctgagttcaattcccagcaaccatatggtggctcacagccatctataatgagatctggtgccctcttctggcctgcaggcaggcatacaagctggcagaacactgcatacataaataaataaatcaaaaaaagataacactttaaagaaaatgatactttgagaattctatgtatagagccaggcggtggtggcgcacgcctttaatctaggtcctcaggaggcagaggcaggaggatctctgtgagttcgaggccagcctggtctactgagcaagttccaggacaggctccaaagctacagagaaaccctgtctcagaagaagaaaaaataaaagatcccaaagggcagtggtatgcagaagacagggaggaagggagggagggagggacagagggagggacagagggaggacagcaggccttttgtggaagcagcacttacaatttctgggcatggctgattcggttgtacagcacattgatctgtagaacgagaagccaggctaggtgcagatgtccaaccaaagccctgccctgcctatcacccctgtcaccccagcctggaccccaacagaggcaggtcccacctcgtatttctgttgcttcagtttctccatcagatcaaatttctctgactcgagctggtggatccattccgacagctcctgggccttctccctggcagaatgagaataactgggatgcagcgagactatgttctgggcccagaaaggttgagacacctaccccaagcctcaaggcaagtctctctgagcttgatacagttggtatgctaagccactatgagcctgatacagttggtatgttgtggaatgttactttaactatgtaaagatgccttacatttgtttactttgtggaatgttactttaactatgtaaagatgcgttacatttgtttactttgtggaatgttactttaactatgtaaagatgagttacatttgtttacgctgtggaatgttactataactatgtaaagatgcgttacatttgtttatattgtggaatgttactttaactatgtaaagatgcgttacatttgtttacgttgtggaatgttactataactatgtaaagatgcgttacatttgtttaggttgtggaatgttactttaactatgtaaagatgcgttacatttgtttactttgtggaatgttactctaactatgggaaggtgtgttgcatttgtttctgctgcatttgttgagttggataaaggtgtgttgctgtttcaccttgcctgcctaaggcacctgattggtctaataaaaagccgaacagccaatagctaggcaggagagggataagcggggctggcaggcagagagaataagtaggaggaggaatctaggatgcagggagggagaccaagggagaaagagagggagatgcctggagccaaccagacatggagtagtcaaaatgcagatgaagagaaacaggttaatttaagttataagagctggtaggacaagcataagctaaggccaagctttcataactaaattatctctccacgtcttgatttgcgaaccggttggtggcccgaaaggaaggcagctacactggtaaattcttgttaagattggaggctgaagttttaaaatatggccactttctgaaacggaggtctcccaaaggagagaaggaagtgatgactgggagccccagaatcggaaagatgtttggtttttatttattgcttatgtagtgtgtgtgtgtgtgtgcgtgtgtgtgtgtttaattttgatttctgagacaaggtctcgtgtaacccaagcttcaatatataggagaggatgacccagaacttctgatcctcctgcctccccctcctgagtgctaagattccacctaaatgagggagagagcactgtcaatgccagactccacaggccatgccaggcacaggacagattgcacccgagtgacattttgaacagagaaagcaacagtggcccagaaaaggaatgtcacttgcctaaagtgacacagcaccaaggctcacacctggaacatcacccacggaaaccttaggagagccacaggtgttgctgtagttccttgtcccaccaggtcccttcgtccttctcagccccaaataaacacacagatacttatattaattataaaactgttggctgatggctagggcttcttattggccagctctgtcttaattgacccatttctataactctatgtatctccacgtggtcttggcttaccggagaatggccggacctgttactccttttggcagctacatggtgtcttccctgtggcccctctctacctacctttcccagaatcctcctcgtctcctagccccgcctatcttgctgcctttattggccaagcagtgtttcattcatcaaccaataagagaaacacatatacagaaaggcatcccccatcacacaggcacttaccggagttggtcctcccccatgtagtcgatgttcaagggttttttcctctcagacaggatcctgagcttcatctcccggccggtctgccgcttcccacgtttctgctcagcctggagggggaggaatcccaagccagggatgggacctggaggccaaatccactcagggtcctacagtcatggccagggcctccaccacttgcaaggggcccaggctgcagccctcctcccccagacccaggaatccaagctccatgcctcctccatcagacacgggagtacaggcccactcttcccttgcacccaggcaccaccagaatgttatctaaagcgacagctgcccccccttacccaaaccccaacgggatccaggctcagtgcccttcctgaggaccagcattctgcagctccccttccctcctccttgtaaacgcagacaccccccccccagggctagactcaccttgaccaggtagcccccaaaatgagcccccatgttggagagaaccttcttcttcttggcatcgtcctcggctcgcttcttggcttcctcctcctctttgcgcatcttctcttcctgtgaaaacagaggggttccctccatgtggccctactaaggaaggcacgagcctgggtagtgcatggctaggctccatagacggggccgcagagggatccactcctaaacttgatatatagttcaccgagccctcaggttggcagaaatcctcctgcctcagcttctccagggctgggatcacaggtctgagcccaccgcaggcagcaaagctcagtcattgtgctatggttttgagtctcttgatccagccatgcctgaactttttaaaatttatatgcagttcatcctttcactaggaaaaacaaaaaacaaaaacaaaaacaaaaaaaccttgcgctgtacattacagggactttctgagaaccacaggggacatcaacggcaggaaaggaaagtattcgtctccaagaaaggggcagacacctaccgccagcttggcctgccgctcccgctccttttcggttctgaatcgctgctgctcagctctctctgcacgacgcctctcctggggtggggttggggatggaagagaggaagatagcggagggggattggaggctaccctctcccccagtttatccctccctccaccagtagacagcaagagaggtaagtgcggtttcttttgttttcttttccttttttgagatagtctcagagagcccaggctggtctcaaattcgtggcaatctaccttccttgtccctctaagagcacgcctggccgaaatacgcattggagtgcaaccacccggtgtccccattgtttctcccggagaccccaaagcacttcacggaggaagggacgaggagggaccgaaggacccggcctccccggcctccccggcctcccaggctcacgatgcgatctttcagcgcaatgagctcctcttcctcctttttgcgctgctcgaagtgcacgtcaatcagagtctgcagctccagtaagtctttctccatgcgcttccggtggatgtcctgcagagggagcccgtgaggcagagggaccagaccctagagccgcccctctccggccccaggggatgatgattgacagccagctgggacggcttccagcagaggtcagcaaagcattcctggctggcaacagggcaccgaggtaaatgcaggcattttcaagaaaggagcaaagaggggcgcctatcagaatgggctcaaggcgctgaggagccagcaagtatgtctggggtgggacacctgtcgcttacatcaaagtccacacgctccccttctgggatcttgggggggatcaaaggaggcaccacaggacggctgtcgggacagaaatgggaagagatcattagcaggctggcctcctcatcccccctcaccacagacagttcaaagtgacagctgcccctgagtctaagcagagaccagtcagaaagtacaccgtctgagcatgttgggtttaataaaacgtgtaagacggtgtgttttgcctgttttgacatacttgtaaaaaaaaaaaaaaaatggacagagcctgatggcacaaacctgtcacccgatctacttgggaatcagagacaagttccgggcctgcctgatctacagtaaagctaaagccatccctggaaacttgtgagacccggcctcaaactaaaaagtaaaaacaggactgggactgtggctcaggggtagagcccctgcctagaatcccccagtgaggggctggggtgtggctcagtggtggagcccctgcctagaatcccccagtgaggggctcgggtgtggctcagtggtagagcccctgcctagaattccccagtgaggggctggggtgtggctcaagacggagcccttgtgtcaaacatataaggccacaggttggacactggacattaaaaggg

SEQ ID NO: 3 - DNA, 22144 bp, Homo sapiens
  misc_feature 1..22144: note = CCR5 sequence
  source 1..22144: mol_type = genomic DNA
gtaaacagagtcctgtaatgcaaggtccggccttggcagccccagcctggagccacagtgagatgtgagccgagggttatgctgggaaaaacctctccctcccagcacctgaaaggctctgcaggcccagcagctcagcaagcaagggtaagggcatggactaacatcttatttcatactatcccttataacacatcctaatgtaatcagctcacaatatgaaattatttcatttctctccagtcattgtttcaatggggccttagggttgactggattctggagggccctgcctagaggagggggtgcattctgtccctatgtcccctcctgctccatcctccacagcacgtgcctagtggtctaccttgtggggaattcttgtacctccctcttctaggcatggactagcattgagaagtgggagaggagtgttaggaaaaagggcaaatatagacataccttgtcttattgtgctttacagatattgtttttgttgttgttgttgttgtttacaaattgaaggtttgtggcaaccctgcctcgagcaagtctattggtgctgtttttccaacagcatgtgcttgttttacatctctgtgtcacattttggtaattctcccaatatttcaaactttgtcattatttctatatctgttatggtaatctgtgatcagtgatctttgatgtcactattgtagttgttttggggcaccatgaagtgcacccatgtaagatggcaaacaatcaataaatgttgtgtgtgttctgactgctccatggactgcctgttcctgagacacaataatgtatatataacaattatatatatatatatttataacaattatatatatatatatatatattttttttttgaggcagagtcgcactctgattgcccaggctggagtgcaatgatgtgatttcagctcactgcaacctctgcctccccaggctcaggtgattctcccacttcagcctcccaagctgggactacaggtgtgcaccatcacacccggctaattttttttttgtatttttaggagagacagggttttgccatgttgcccaggctggccttaaactcctagactcaaacaatccacctgcctcagcttcccaaagggctgggattacaggcatgagccactgtgcccagcccaagacacaataatattgaaattaagccaattaataaccctacaatggcctctaagtgttcaagtgaagggaaaagtcccacgtctctcactttaaatcaaaatctagaaatgattaagcttagtaaggaggacatattgaaagtcaaggccaaaagctcacctctgcaccagttagccaaattgcgacttcacaggaaaagttcttgaaggatatttaagctctactccagggaacatgcaaatgaagagaaaacaaagcagccatattgctaatatggagaaagtttgagtggtctggagaaaagatccaaccagccacaacatttccttaagtcaaagcctaatccagagcaagactctaactctcttcaatgctatgaaggcggagagaggtgaggaagctgcagaagaaaagtttgaagctagcggaggttggtttgtgaggtttaatgaaagacaacatctccataacataaaaatgcaagatgaagcagcaagtgcaaagggagaagctgtggcaagttatccagaaaatctagataagataattgatgaaagtgtctacacgaaacaacagattttcagtgtagacaaaacagtcttatgttggaagaagatgccatccaggactttcacagctagagaggagatgtcaaggcaagctgcaaagctccacaggacaggctgactctctttttagaggtgaatgcagctgatgactttaagttgaagtaaatgttcatttactattttgtaaatcctggtgtcattaagaattatgcgaaatctactctatctgtgctccataaatggaacaataaagcctggatgacaacacatctgtttacagcatggtttactgaatatttcaagcccactattgagaactattgctcagaaaaaaagattcctttcaaaatattactgctctgcaccatgtcgatcaagagctgtgttggagatgtacgagaatattcatgttgttttcatccctgctaacacaaacatccattctgcagtccatggaccaagactttcaagtcttattaagaaatatatttcataaggctattaagaaatagctatatatatatatatagccttatatagtttatatagctaccattgatagtgattccattgatggatctgagcaaagcaaattgaaaagcttctggaaagtagtcattattctagatgccattaggaacatttgtaattcatgggaggaggtcaaaataccaacattaacaggagtgtgaaagacattgattccaacccccatagatgactttcaggggttcacgtcttcagtggaggaagtcgctgtagatgtggtggaaacagcaagagaactagaactagaagtggagcctgaagttgtgactgaattgccgcactctcatgatcaaacttgaacagatgaagagttgcttcttacatatgagcagtgaaagtggtctcttgagatggaatctcctcctggtgaagatgctgtgaacacggttaaaatgacaacaatcgatttagaatattacataaatttagttaataaagcagtggcagggtttgagaggattgactccaattttgaaagaagtgggtaaaatgctatcaaatagcatcacatggtatggagaaatcttttgtgaagggaagagtcgaccaaggtggcaaattgcattgtcatcttattttaagaaattgccacagccacccccagctttagcaaccaccaccctgatcagtaagcagccatcaacatcaaaacaagaccgccatcctcttcagcaaaaacactatgacttgctgaaggctcagatgatggttagcatttttagcaatacaatatttttaattaaggtatgcacattggtttttctgacataatactattgcatacttaatagactacagtataggataaacacaacttttatatgcactgggaaaccaaaaaggttatttttgagatatttgctttactgtggtggtctgaagctgaactcacaatctcaccaaggtgtgcctgaacctctttagctaactggccactgccacagtccactctgtgttggtcaagatgccccagagtggcaggcacactgtgtggtcacatccaagggcctagatatggtgggggctccaaatggatctagatatgtgagatctctctttgatttgacttcttccaacccaccattttctgggtgctgggctcatctcacccagaaagtaggacccaatgtgacagttcctgcccagttccctcctgtggtagccacttgacccaggggcactcttgatccttgcagcctcacttacacaccctatctctacccctattaactctctccaatccccactccccctgctcagcttgtctgctgcccagtgggggccccacccatgctggcctctccttttgcaagtccccattcctcatatggtttcttcagagcccctttctttggctttgaggagagatgccctcactcgcttccccaccaatcctgcccacttctacaatccattcattatcctaattgcctccgtatacagactggagtgagaggagttgatgtgatgggtgtggatacagggctggtgctgtcatcttctagtaagccctgggagaggtgtctgagcccaggtgtcagtggttttctttggaactgtgagtgcataacacttctttgccttcagccttaggccatagttgctagttctgggacaaccagaaaagccctacataatctcgtgttatgtgcagagctgagtatagagctccaggtatgatctgactcacttaagatcacagtgagtctattgtattgttgaactgttagcttagacatctgttactgtacctacatggcactagcctcacgcctagacaccgatctgaaagaaatcccctaaatgcatagagaagacttctcagctgagctaaggggctcccaccaggtttgagcctatctaatgaatccatgaggtagacagcctgcacatgtccacttggtttgatgaattgcacaaatccctatgggggatgtggttcatgggctgggaagtgggttaccctgggaaaggtctacaggacagaggcagggatggagacaacagcatggtgagttcccaacccacccacgatgataggtgtctgaggcagaaggtaaagaggctgtcacctggtgggtgtcataagactcaagtgtcattgttgaggcacatgggtaacaaagcgtggcactggatgggggtagattcttcctatttctgtgaggatcagggggactccctggctctcctgctaaaggtggctctagggacaggaagagtgtacttcttgacagggatgtcagagcactgatggtgacaatcagtgtgacactgctcacatgactgaacaaccgagaagagcccgactgtctactgaacaacgggaagagcccgactgtcaatgacggagctctgttaaatatagttaaggctattttgttgaatgaatgaagccagacaggaaagaggacagtatctttaatccatttatagaagttaaagacaggcttatttaatctctatgaagacagagtggcccttacctctgggtggagcaaaaggcaccttctgaagtgatagggatgttccttatcatcttgatccggagtggtagttacatgcatgtgtgcatatcaaaactcaccaagctgtaccactaagtgtgttcttcctcaataaaaataataaagaactacacttataaagaattttttaataatataggaaaatgtctacactataatctttagctaaaaaaaaaaaaaaaagaagccgcctacagaatggtatatgcatgagaacaattaatcgaaaagtgcatgggaaaagtcaggattgaaacatcatgttttaaaagacattgttttgatactgtgagaatgtacctaagtttttccttttttctgtttttcccaattttatacaatgagcatgtgttggttttataattagacattttgtttgtttggtttggttttgagacacagcttgctgtcacccaggttggagtgcaatggcccaatcttggttcactgcaacctccatctcctgggttcaagagattctcccacttcagcctcctgagtagctgggactataggggcgcaccaccacatccagctaattttgtgtatttttagtagagatggggtttcaccatgctggccaggttggtctcaaactcctgacctcaagttatccactcgccttggcttcccaaagtgctgggattataggcatgagccaccgcacttggcctagacatttgtttttaaaaataaaagattcatttgctctttttacagcccgtctcactgttgactgatattgaccaggagtcaactcaggccccagggattttcacaacagctgctgtatggcagggtttctgctcactgtgctcatgtagttggcccttgcacccaaagtgaataattaacattctccccatcctgttgacgatgctctgaaaatatggtccagaaatggtgtgagcaaggagacagcaaagcaatgcttggaacataggtgcagtgactagacatggggcagctgtttaaagacaaaaaggccccaaaaaggagggatggcacgaaacaccctccaatatgggcatggagtctagagtgacaaagtgatcaaaagttcatttcctatggggtgtccgaatgtacttaataataaaaagagaacaagagccatgcaaactgagagggacaaagtagaaagagtagcagacaccaagcaactaagtcacagcatgataagctgctagcttgttgtcattattgtatccagaacaacatttcatttaaatgctgaagaatttcccatgggtccccactttcttgtgaatccttgggctgaacccccctgtcctgagtggttactagaacacacctctggaccagaaacacaaaagtggagtaacgcacactgcaaagctgtgcttccttgtttcagcctgtgaatcctcaccttgtttcccatctagcctatatttttcaaactaacttggccatagaatcatgtagtatttagggtggaagctgccccaggtctagcacgtcatttaacagatgaggaaatggaagcttgggcagtggaagtatcttgccgaggtcacacagcaagtcagcagcacagcgtgtgtgactccgagcctgctccgctagcccacattgccctctgggggtgagtatgtcttcacatcctccaataccctaatgacagacaaacagaacatggcaaagcctcagctctgcatggtgaaagtaagaaccagcaattgccacaaacagaaatacagtgttggtccggcagcctccgggggttctgcacaagtggattaccagtgaatacaaggctatctatctttcgaaaaaccaaagttgtatttatgctatctattttctataaaattttatattaatttatttgttacctatttttgaactctttcaaaagcacactttatatttccctgcttaaacagtcccccgagggtgggtgcccaaaaggctctacacttgttatcattccctctccaccacaggcatattgagtaagtttgtatttgggtttttttaaaacctccactctacagttaagaaaactaaggcacagagcttcaataatttggtcagagccaagtagcagtaatgaagctggaggttaaacccagcagcatgactgcagttcttaatcaatgccttttgaattgcacatatgggatgaactagaacattttctcgatgattcgctgtccttgttatgattatgttactgagctctgttgtagcacagacatatgtccctatatggggcgggggtgggggtgtcttgatcgctgggctatttctatactgttctggcttttcccaagcagtcatttctttctattctccaagcaccagcaattagctttaccttttcagcttctagtttgctgaaactaatctgctatagacagagactccggtgaaccaattttattaggatttgatcaaataaactctctctgacaaaggactgctgaaagagtaactaagagtttgatgtttactgagtgcatagtatgtgctagatgctggccgtggatgcctcatagaatcctcccaacaactcatgaaatgactactgtcattcagcccaatacccagacgagaaagctgagggtaagacaggtttcaagcttggcagtctgactacagaggccactggcttagcccctgggttagtctgcctctgtaggattgggggcacgtaattttgctgtttggggtctcatttgccttcttagagatcacaagccaaagctttttattctagagccaaggtcacggaagcccagagggcatcttgtggctcgggagtagctctctgctgtcttctcagctctgctgacaatacttgagattttcagatgtcaccaaccgccaagagagcttgatatgactgtatatagtatagtcataaagaacctgaacttgaccatatacttatgtcatgtggaaaatttctcatagcttcagatagattatatctggagtgaagaatcctgccacctatgtatctggcatagtgtgagtcctcataaatgcttactggtttgaagggcaacaaaatagtgaacagagtgaaaatccccactaagatcctgggtccagaaaaagatgggaaacctgtttagctcacccgtgagcccatagttaaaactctttagacaacaggttgtttccgtttacagagaacaataatattgggtggtgagcatctgtgtgggggttggggtgggataggggatacggggagagtggagaaaaaggggacacagggttaatgtgaagtccaggatccccctctacatttaaagttggtttaagttggctttaattaatagcaactcttaagataatcagaattttcttaaccttttagccttactgttgaaaagccctgtgatcttgtacaaatcatttgcttcttggatagtaatttcttttactaaaatgtgggcttttgactagatgaatgtaaatgttcttctagctctgatatcctttattctttatattttctaacagattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccggtaagtaacctctcagctgcttggcctgttagttagcttctgagatgagtaaaagactttacaggaaacccatagaagacatttggcaaacaccaagtgctcatacaattatcttaaaatataatctttaagataaggaaagggtcacagtttggaatgagtttcagacggttataacatcaaagatacaaaacatgattgtgagtgaaagactttaaagggagcaatagtattttaataactaacaatccttacctctcaaaagaaagatttgcagagagatgagtcttagctgaaatcttgaaatcttatcttctgctaaggagaactaaaccctctccagtgagatgccttctgaatatgtgcccacaagaagttgtgtctaagtctggttctcttttttctttttcctccagacaagagggaagcctaaaaatggtcaaaattaatattaaattacaaacgccaaataaaattttcctctaatatatcagtttcatggcacagttagtatataattctttatggttcaaaattaaaaatgagcttttctaggggcttctctcagctgcctagtctaaggtgcagggagtttgagactcacagggtttaataagagaaaattctcagctagagcagctgaacttaaatagactaggcaagacagctggttataagactaaactacccagaatgcatgacattcatctgtggtggcagacgaaacattttttattatattatttcttgggtatgtatgacaactcttaattgtggcaactcagaaactacaaacacaaacttcacagaaaatgtgaggattttacaattggctgttgtcatctatgaccttccctgggacttgggcacccggccatttcactctgactacatcatgtcaccaaacatctgatggtcttgccttttaattctcttttcgaggactgagagggagggtagcatggtagttaagagtgcaggcttcccgcattcaaaatcggttgcttactagctgtgtggctttgagcaagttactcaccctctctgtgcttcaaggtccttgtctgcaaaatgtgaaaaatatttcctgcctcataaggttgccctaaggattaaatgaatgaatgggtatgatgcttagaacagtgattggcatccagtatgtgccctcgaggcctcttaattattactggcttgctcatagtgcatgttctttgtgggctaactctagcgtcaataaaaatgttaagactgagttgcagccgggcatggtggctcatgcctgtaatcccagcattctaggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataattcattatattcttgctctttcagtcaagtgtacatttagagaatagcacataaaactgccagagcattttataagcagctgttttcttccttagtgtgtgtgcatgtgtgtgtgatgtatacaaagagagagataattgtatttttgtattttcttttaaataatttttaaaattgacccttttcctgagacaaattgccagaatagtttgtatttagagatggtacctctaagagtaaggttgctggttgctgagcaattgacttgaaaacttttaaaattcaaattttaattccactactcaaaagaattgccatgttttaaaaaagagaattggtgccataagttagttgtctatgtttgaaaatgaagaagatatgcaacgtcatggcctggtcacttacccgcagccctgagttgtaggcacatcatatgtgagaatgaggatgcttttctttcatttaaaatccctccccaaaacttggctctaattgcagtcatgacaatcatgtacatttggatttatgtgcacgagtctcttaccctgagagaggacaggtgctacaggtggaggggacccgtctgggtcacgttcacattttgaacatgctggttttcagtcactgcacactcatctcccagcacaggtcatgggcagcagatgcaaaagctgcccgtggtcctatttggaggtgcatgaaatgagcagaagacagaacagcttgatctgactagaagggcagcttgtccctaccaagacttgaaggattgcctttcatctgttagggtaaaaggtagaatgaaccaaggaagggcaggagggggctggggttagggtagaaggaaggggccatggagaagggagatccatcccataggaggaaggcagtgcggcagggaggtttgaaggtatcagcttttgtggctgacatacatgcagtcatgtcaattgctcgtttttccttttccatcttattaaatgtcttccaacgttagcacgaagaaaagctatttgcagtgttgccagcctttccagagcccgtccccattacctccccaggcccatgcctttactccttggagtttcaactcacgaccttcaggatctgactttattcaccaactctggggtgaacgtaccttctgtctccacccagaggtctctatcaaagaggagattgcatgccatggataaagtcaaagtagaggtgactgtccttaggaagagtaatgtgaaaattcataaactgggattctgtttacattttgtactccaggggttcttagtttaaatcgctctgaataaattaagatgcaatggcatttcaactgttatgattaaatttacaaatcatttattttctatcacggggagagatagagctccaaatgcaaacataactgctcaagtgttaacacttataatgaaaacataagaattaccaccaactaccctgggggctagaagcagaaatgtgaaccagaaaacaaatcatgaactttcctttttttttttgagatggagtctcgctctgttgcccaggctggagtgcaatggtgcgatctcggctcactgcaaccactgcctcccgggttcaagcaattctcctgcctcagcctcctgagtagctgggactacaggcatgcaccaccacgcctgggtaattttttgtatttttagtagagacagggtttcaccgtattagccaggatgctctcgatctcctgacctcgtgatctgcccgcctcggcctcccaccgaagtgctgggattacaggcatgagccactgtgcccggccaacaaatcatgaactttctaactgcagttccttgtagcttgttaacacatccacttacttattgtcagagtacgtggagattttccacaaccctcggggataaggctgaacagaagaggcaaaaacgtgaaaacatttcgatagctcctatactttgaaataaaattcactgtaaaagttgcttgtatttttccaaaacagagtcaacccttaatatttaagattctgtatacaaatacatatttttatataattaatatatattgtcatatgacatatatctttatattaatatgcatgcatataatatatatttccttcctaattttctataagcaattttacaagactgacttctatttgcctccttattgttactacgtggtttgataatccgttttgtgtcattgtgattctgtcatgttttggggacttatttttgtttctctgggtggtcactagtttttttaaagcattcatggaagagtgtgaatcttttacaagctaggaagccatggcaagccttgggtcatactgcccccgcgaggccacattggcaaaccagcaagggtgttcaacttccagacttggccatggagaagacacacgaggaggcttttcacattcagctctttaatgtttgtctctgccggcaccatcccagttgtgaaaaagaggtatttccacagcggctcagggtaggtagtgcacagctcacattcatcatttctgaaaaccgagaggagtctccattcggggtacaggttgatgcctgtcgtggaatgaaggttccaacacccagaccaatctctgcagtgtgctgctctcatgagcttgcaacaagatcagaaaatgttttgtgactaagcatttttcatattgcataaaatgcttcaagctcctcccttgtttctctctataatcctgtatatctgatgattgtgggtaccaagtgtttgaaataatcaaatgtgatttgatgttggtaaatttctttttttttttttttttacttctattttttttattatactttaagttttagggtacatgtgcacattgtgcaggttagttacatatgtatacatgtgccatgctggtgcgctgcacccactaactcgtcatctagcattaggtatatctcccaatgctatccctcccccctccccccaccccaccacagtccccaaagtgtgatattccccttcctatgtccatgtgatctcattgttcaattcccacctatgagtgagaatatgcggtgtttggttttttgttcttgcgatagtttactgagaatgatggtttccaatttcatccatgtccctacaaaggacatgaacatagcaaagacttggaaccaacccaaatgtccaacaatgatagactggattaagaaaatgtggcacatatacaccatggtaaatttctttatcattcgcactctcctttctctattattgttattgtaactgaaccgcagattagtcactcattgcttgcagaatccaattaacaagagcgaggtcagatataaagaaaatgatttattccaaacctccttcagggaagaggtgcagcctcctgcctctaaatgcactgcttcgccaggcgtggtggctcacacctgtaatcccagcactttgggagaccgaggagggcagatcacttaaggtcaggagttcaagaccggcctggccaatatagtgaaacccctgcctctactaaaaatacaaaaaattagccagacgtggtggcgggtgcttgtaatcccagctactcgggaggctgaggcaggagaatcgcttgaacctgggaggtggaagttgcagtgagctgacatctagccactgcactccagcctgggtgacagagtgagactctgtctcaaaataaataaataaataaataaataaataaataaataaatagtaaatgcactgctttgcttttggagcagaaagcaggcactttgaaaaggcaggggaggaagtgagcaagggcagggggtctgcacactggcatggtgcctgatctatccaggcagttgaattggcactttcataggcagaaataagttgaaaaagtggcctaaaactctctaggtgggagtggatagtgggcatgccttcaacctgcctttctggagggtgagttccatggcaaccccctgaagggtgagagttccatggagatcatgctttggtctgtaaatcagctgttaactctctagaaagttctgtcttggagcatatagttagatgaacttgccctgtaaagaatgtctggtgaaggggaagtaaaaggtgagatttgcatttctaaagggctaagtagaaagtggggtacaagaggaaaggagaaaagagaaaataatttaaaaaataattgtaacttattcccttttacttagaaaaaagggaatactcagttacattatcacctcgtttacatcaaaccctcttatggaatcctatggtttgaaaacaaaaaggttgttgaggaccagtgagcccaacccctttgctttataaatgaagagcattgcctgccctaagccccagagactctgatgtcgtgggtctggagtgggctccaacagcggcatgttttgatggtgcttcccagtggcacgccagcgatgagcctttgagtagggaaagtaggagcactcgtgactcccttcacgatcagcacctgtgtgctaataaattcacaaaagccaacatattggagtcactcagggagttttacaaatagtgaggttaaatccaacctcaaatagttctgattcgatctgcctgcattgctgccctgtggttccccactgtagaagctccccaggtgattctaagtgtagccaagtctgagaaatactgcctaaagcctgttggactgacagcaagggctgttgtctgagcaagactttgcctggcctggggtggcatgtgcaccaggaagagtctcaactttcataacagaacattccccaagctggtttttttaaagcatgtgaatctagacttcattggcaataccaaagatctgtatttgaggctccaagtatttcactttcatttttggttttgggttatgttttcacccttcctttccaagtgaaaagtaaacagaagtgggatgtctggcgcccatgctgagcttggcaacttcaaattcaatagagaagaagtctcttgtatagaaaagggcctgtctgagatgtttctcaaataaatatagattttgcttatgtggctaaaggattcttctccccccatttccttatccctgcagtgagccatccttcttaactctttccatgaaagcattattcctgaagaactgggaactcatgccagccctgatcaggcaatgataattctgcagagaattagaatttagatttaaattgtcaactcttatacatcctggcatatggtttaaacacatgtacacacacacaaacacctcctactatttactgaagagcagatatctgataacttaatctttttggttttgagtcaagacaattcctccttttgaaactgcataccgctgaatataataaaatgtaattaagattaaaaataagaaactaatgggagaatttcaatattgtctatgttcactttaaaattcctctacttaggtttactgccattaccaaagactattcaaaaatcctttttaggagaatcctaatggtttcctgacatataatcaaataaggactctgttgattggctaactcaatcttcctgtgccaaaaagcagagcccagcagagaagagggcagggacttgaaagtcagactgactcgagttccagccttggggctgtgggagcttgggcaagtgacttaacgtctctggctctcaggatctaaaaggatttccagtagtaatttggggtgttactgatacaggagctaaaaagaaattatttaggtggttagtgagggtcagagagtcctcggtaagatttgccttttaacaaaaagcagccccaaaatcatttgtttgctaacaaagagaagcctgtaaaattgagctgcagacatagataagcaagctggaagcttgcacgggtgaatgccggcagctgtgccaataggaaaaggctatctgggggccaggcatgttcaacatggattctccatcttcccttttctttgtcaaccaagtgtacagtaaaggaacaggcaacatggcacgggccaggtagagaacccttctgcataataaaagattagggtgagatggccagcttcttcccgtgctatgtaaatggcatacctggtccaaccagtcttttgggccctgtgtaaatcagacaccgcctcctcaagttagtctataaaaccccatgcattttaccgtgaaactgggagatccactcggaaccccctcctgcacgagagaccttttctcttttgcctattacacttccgctcttaaactcactgctcatgtgttagcatccttgatttccttggcatgaggcaacgaaccttgtgtattaccccatacaaatgatgctgcttcattactaatagcaacctgacagggttgtgttggggtataaattatctagaccagggagatccaatataatttttttgtaatgacgggaatgctttgtatctgcatcatccaaaatggtagccaccaggccagggtgaaatgtggccagtgtgactgaggaactgaatgttttccatgatttaatttaaatgtggccaatggctactgtaggagacagtgtgagtctggcatattataaataataaatattaatataatttgaactttggcatcagtgtttcctagatttgaattactatgcaagttgcttactgtttccaagcctcagctttctaatctgtaattggggctaataatagtatctgccttacaggtttgttcagaggataaatgagaaattgcatgttgagggcttaacacagtgcctggcacataaaagctctggtaacagttagccactttaataatttgctaataatggctatttcttcttcagattaggatgtgctcccccaaacagtgcacttagacatagcgggcaatccagctcactctctgcagtgagagagaagcactggccgaccagagtcagccaggggctcatgggtatgaaatcaacagcatgattttgtaagtaatggatggaaagggcctcacaactttatggcactgtgttcaatttgcttggtcttctgtagctccttttgaaagccttttagggtggattaacctgctaccaataattctggtcagatgtagactccatagctcaaagcaaactgagagagtgagggcagcaggccaattccccaccccttccttctggactctgacagaagcttacactcaaggaagagcaagtaggaattaacgtgttaagagctaggtaagcaaaacccaatgagaagttctggcaaagccccatgggcaggggtggcttaggcacaggaaacaagtaggatttcataccacgcgcctcagtctacttccggggccctcatcctcagctgtgcctatgcaaaggagagcaaccaataaaccccaccgccactctcctactgtggaggccagggatggccaggggtaagagagggatgggaagtgtttcctccagccgtcctctgagaaggagaggaaactgggcagagcttctgtcctccttcaagcagaaacagaaacaaaagaaacccctaagggggttcttacttcccctctagttcagttgtgcactaaccatctgcagctcaacattcagcattcattcattgattcagcaaacattgaaggagggccagctatgtgccagatgccaactcatgccatgaaagagagtccctgtccttatgaaattcactatttagagagaaaagcaagcaaaaaggcaaagtttgaaaagtactgttgaagtggcatcattgtctggggtgaatacctgaggtttgtggtctcacgccaagggaatcaaggactcaggcacacaagaagtgagtttaagagcagaggtttaataggcaaaagaaagagaaaaaagaatagctctcttgcctgcacagagagaggggcacctgagtggatcttcctgttttgtggtgaaatgcaaggcattttatagacgagcttgaggaagtggtgtctgatttacttaggacccgagagattggtcagaccaggtgtgatgtttacatagcatacaaagaagctggccatcccatcctaatcttttattacgcagacggggtctatacctggctggtgccatgttgtctgttccttactgtacacgtggttgacaaagaaaagggaagatgaagaatccatgttgaacatgcctggcccccagatagtcttttcctattggcacagctgccggcattcactcttgcaagcttccagattgcttatctatgtctgcagcccaattttacaggttgctctttgctagaaaagaaatgatttgggggctgcttttcattaa

SEQ ID NO: 4 - DNA, 20020 bp, synthetic construct
  misc_feature 1..20020: note = Synthetic: hRosa26 sequence
  source 1..20020: mol_type = other DNA
accatttaaacctcaaattaagcaacccacagaaccaggaagttcaaggaccatgtctgttttcaccacgatgtctttccccacccccccaccccccactccaccccccactaagggcagggtattgtatctgccagactgggtatttgttgaacaagcgagtattttcgcctattagcttagtttttaaggaaatcattttttacttgattcatcatagctttaattctattacatactacaataaaaatttgacaagactgatacaaatatgtagtgggcaatagtttgccgtcttcttccctagtatggtgtttttcaatctggtgactagaataggcagtgggctataagcaggattcataaggcctggagctgagttatatgtgacactgccacctattcattgtgtgaccttggttttaaccttcaaagtgggtctcctggactaaaagaatgtgaaaagatggggaaataaatctgtaatctgaacatggaatgacttagttacagaccagacatattgttactgggaatgaaaaagtcaatatatttgaggggaaaaaaatgtaaataaatattgagaaagattttacaaatctaattaggggaagatagttatctcccaatactagagggtaccagagtgcttttaaggggaacatttggttacccttatttctttaaaaaatggcagtttaggaaattctgccctaactgtagtcccaatgtcagataggactcaggtctccactgcaaggaccaaaatgttaagttgaagactgaaaatgggaaaatttggaaatgtctttggaacctcaagtacataaaagcctgtaagtgcttcatactcattaacaacataggcatagaaaaaagatatccttattctcaagcatagccttttctaataagttcatgttagatgtcatgaagtttagtgaggagtgaaaatctatgaggaaaaacatgaaccattctactctggcaaaagttcaggacaaacaccataggcctgtataccaaattttaaaccatgttgaataatgtgaaaaaaagcatcacattgcttatgaaaggctttcctgtcgccccttaatacttctgtctcaggctaacatgtttgttaatgagttacagtggtgaagttaaggaaatctgcttcctgtcctagcatgcccattatcccagccatacagatttaataccaggagtcactttaactccatgaagtcattcaacaggtacttgagtatttactatgtgcgtttgtgctagagtagccatttcttaaactttgtggcctcagagaatcccttcacattcttaaagattgagggccccaaatcactttactagtatcttaccatattagaaagtaaaatatttaaatattatagcaacaaatgcatatttttaaaaaaataggtgcattgttttacattttggcaattgtttccctaaatgtctgacttaaatagaagacaactgaattgtttcttttgcattcaattggttacaatgttactagcagttttctataatctcacgtattagtcattaggaaaatatagcttcactgagttatgtcaatctcccaaatgttggcccattttactgtatactacctaaaatatcattggccgggcacagtggcttaaacctgtaatcctagcactttgggaggccaaggtggatcacctgaggtcaggagttcaagaccaccctggccaacatggtgaaaccccatctctactaaaaatacaaaaattagccgggtacagtggtacacacctgtagtcccagttatgcaggaggctgaggcaggagaattgcttgaacccaggagacagaggttgcagtcagccagatgtcccaaaaaaaaaaaaaaattattttcaatatcactatctcatgaagtattgggaagctgtcaagttcctgatattagacacaagttttcccgaaattctgattttgactcaacgtttggattttatcattgaaaacaattgctgtcagttgttaggctccaaggaaatagcagataattcagcttacatgagtgctttttcttgagacaaccatttcaaaaagttatgtactgtagggtttaagatttaacaaaatgtcactgctttcacaaggacattcttgagtgaaactggtttttttcttttgtgggggttcacaccacaaatgcatggcagtgaaaaataacttagagtttgatgccactgccacagtttgtgccaaggtgcctgaaattttacttttacctactgttgccttatcaccactcttatgtcaacatatagtttagcataaaccatgagattttaaaaagttattctacacttgcattatttcaggacatgtgtttgttgccaagctttcacgtaagagtatctttaactagttggtgctgatgcctggcaaatacaagccaagtaacaagtccagccatgtttatgcacgcatccattgataacgtattagcacagtcagccctccatatcctcaggttctgcatctgcagattcaaccaaatgtgcttgaaaatatttgagaatatttaatacaatagtacaaatataagtacacaatgtaacaactatttatatagcattcacattgtatgaagtataagaaatctggaaatgatttaaagtatatgggaggatgtgtgtaagttgtatgcagatacagcgccattttataaaagggaattcaacatccttgggttttggtgtccttggcaaatggccagcaagggtgggggagtgctgtccagaaaccaatcccaaagcaagggacaaatatatacttctacagatgagatattaactcagaatccatgtctgcacacacatctttaatgacaagttgctttatcactcaacagcggcactactatgatcttttacttacacttcagccagcagagacattcagcagtgtgagattttttaagtttttcagctattgtgtatgtttctagtgtataataaagtaacttatcctttaagatacttaagtagcttttcatttctagctttaaaacctgtttttttttttttcccagtagtggcatacctgcattaaaaaataatgccttaccaaaaaaagcactctgaatgattggtttcaaaatgatgccacaacataggtggcaccaacactattcaagatcattccattcccatctctaaaaaaatttttggctgggtatggtggctcacgcctattaactcaacattttgagaggcccaaagcaagatcactcagggctaggagttgaagactagcctgagcaacatggcaagatcctgtctcaaaattctttttaaaatttttttaaaagcccaggcttggtggcgcatgcctatagttccagctactcaggaggctgaggcagaaggatctcttgaacccaggagtttcaggctgtagttcactatgatggcagctgtgaatagcctgggcaacacagcaagacctcatctccaaaaaaagacaaaagaactaaattattctactgcagaacatgattaggtaaatatctccaaagcagaaagacaggtttcatattttcgttagtttgagtcagtccttccaaatcaaatcttgttttttattagtatacagatggtatagccagtaagtaaatgagaagcagtctttttaagccgatccattcttaaatgaaaaaatatataaatattttagaataaatttattaaattctaaagttgtagaatttttaaatttggatattttgggaaaatatttaaaccactattgcaaacaaaacaacaaaatgtacttatgtttatacttaggcacaaagaaaactacagtattttaaagtaaccattacacaatattgaggttgcaaagattactgaaggcataacctaaaaaatgagttgatttctaaaaatgggaaaaaggaaaaaaataatttctaaaaacaagtatgcatacctaaacctacctaatgacaccttagaaaattcaagtatagcaccattcattaacatcaatgaggatgtcatcacacatcatgtagcctctgcacaccgtgagaataaatgaaaaagacaggcatcttgctatcatgacaatagttttgacctcgcagacctctctgtgcttacgcaacggataaagccataagaactgtcctgccctcaaggagcaacctaaagtaggaaaaaaaaaacaaaattacacaattattatttacaattgtgagaagagctcttgacaacattcaaatggaggatacagtgtagtaagggtgagggtatcaaggcttccttgagaagtgatgttttgaggccattctttcttctcaataactggtatttggttcctgaatcctttaacttccttaccattgtcactcctaagccaaatctcattacgtcatgtctagactactgttaagagaaccacttaagtggtctctgcagccctcaatttattggtgttatctatgggaaaattgctcaaactctaagccttagtttcctaccctataaaatggggttttatatacaaggaacatactaaatacacaggtatacctcagaaacacggcaggctcaattccagagcactacaataaagcgaatctcatgaatttgttggtttcccagtgcataaattatggttacactataccatagtctattaagaagtgtacaatagcattatgtataattgataaatacatcactgctaaaaaaatgctaacaatctttttgctggtggagggtcttacgccaacgttaacgatggctactgactcatcagggtggtggtggttgaagattacgatagctgtggcaatttcttaaaagacaatgaagtttgccacactgacatcctttcacaagacttctctgtagcatgtgatgctgtttgatagcatgatagcattttacccacagtagaacttttttttccttttcctttttttttttttttaaacgcaaggtctcactctgtcacccaggctggagtgcaggggcgccatctcggcttactgcaacctcctcctccctggttcaagagattctcctgcctcagcttcccaagtagctgggactacaggtgtgcaccacacctggctaatttggtagaggtggggtttcaccatgttggcaaggctggtcttgaactcctgacctcaaatgatctaccagtctcggcctcctaaagtactgggattgcaggtgtgagccaccacacccagccagtggaacttatttcaaaattgaagtcaactctctcacaccctggcactgctttatcaaccaggtttctgtaattcctaaatcctttattggcattttaacaatgttcacagcaacttcaccagtagattccatctcgagaaaccactttctttgctcatccctaaaaagcaactcctcatccattcaaatttgatcatgagattgcagcaattcagtcacatcttcaatgctttacttccagttctagttctcttcctgtttccacacctgcagtacacaaaaagcattcaataactattacttcatttcttctacctatgtttccattagcttttgcctatagtacgcactagagtatgttaccattatttgttataagtagtacctcattattacactattcgtaagcaatacctcaaggtctaagattagattttaaatcaaggtcagtaaaaatagaaaaggctgtgaagactgttgactgactttaccagaatccatacactagaggtgagattagttaggtgatgaaataaccattctataaacatgatctgaaactctgttactgttgtcagcaggaaaagccaatgttacatatgtttaaaaaagaaaaaaaaaacccaaaaccagaaaacaaaaggtgacaaagtatcaagacaaaaggtcactgatgactgatctctaggaaaagctggaaagcaggattattaaatgtaaccacgactaagataaaaatcagagacagaaaagtctttgtcaccaagaagatatactccatgagagagcagaaacaattcatcaggtttaaccctgctctagataaaataaaactatctgattcaatactcacacttctctaataatccaatacattatcccatctcaagaagagagagtcacagataagaaaaaaaaggcttcttgagaagtatgtgctctaatataaactaatatgccactaagaaagcaacctgcaaagtccagtaccagacttctggatttgtgacctaacaaggtgctctacaattaacctaacagtcaaaccagagtgttgtaaaagagaattatgtaattatgccaaacctccactcacaaaaaatatatggaagtaacctaagtttacattttgcaaatctcacacacacactagccctgacaaaagtttcaccagctttctcatccaagtacaagcgtgtaatatacttaataaatttgtcttataagggtaagaaatagtatgtaactacttgaaaaggagataggtagctggttaatttaaacaaaaagcccaaggaagtaaggtgcaggaaaaggataactgcaatgattagtacaggaaacccaaagaagaactgaatggtgggatagatgtactcagagaccatgaggcatcagtttcctctatgaatagaatattaggagatgtaggttaaatgggaccctgaagtctctcccaaaaagccttgtttatatgttttctgagcttaactattacttgagaatcaatttcacgtataaaccaacaaaactaacatttattgagcttccagctctgtgcttaggcactgaaaaatcactttccttaaggattgcaattaagcaggagaaacacaaataaggtgaacttctcttgttcgaaagaatatatttcaacattccttttaaaaggaaaacctgacctgcaagtttccaaaaatattaattactattcctctttgcctctcaaaattcccattctgttattttttaggaggaggaaaaaacagttcatttgaggaaaaattgagggtcacatactatacaattgagaagagtttctctgaaactgtaatcatttttggcaggtaaataggcatatccgagtcagcaaatgaacttgaagatactgagttatactgcctgccctgtggggttccaccttccccaaaagaattcagaatttttgggtgatctgagaatctacattaagacaactgtctccacacacaggaggcctgaagatcgctgacataagggtctttttaaaaagtatatttaatggcctagggcggtggctcacacctgtaatcccaggactttgggaagcttagggcaggaagatcacttgagcccaggagttctaacctgtgcagcacagcaaaaacccatctctacaaaaaaaaaaacacaaaaaaattagctgggcatggaagcgtgtgcctgtagttccagctactcaggaggctgaggcaggaggatcacttgagcccaggaagtcaaggctgcgtgagccatgatcatgccattgcaatccagtatgtgacactaagactccgtctcaaaaaaaaaaaaaagataattaaaatgtgtaagatactgtattagcaatataaaaagcatttggtgttaaaatgttggtattataattcctcaggataaaacttactttgtgattgttttctataactcaagatatgatgcttagagctcctccaatcaagtgtttccaggaagtgaaaacttgtaggacagaaatttaggctgggttcatttgtatcacacagacctattcttcattcaagttctgatatatttaactatgtagctcctgtaacagtttaatggaatctcacctccctaaaattcattatgcatttttttttgaaatccaaactcattaacgcttgctttcactgttgtccaaggcaggcacatctttaaaaatggtttgttggacttagctttcagctaaatatataataaataaaacaaaacaagcagttaaatgaaatgtaatgggccagagagcttcagcttttatttccttactgctcagtaaaaagagaaaaccatcaatgtccacgtattctgtaatccacagaacaagtccggggctacagctatactgtccacagttgcaattcaaattagataaaaaataaaaattcagttctttagtcataccagccacttttccaatgctcaagattaataaaatgtcaaaccataaagacatttacatgtcgctcactccatttacttaaagttggctagacatcagagtatactaggagctcaggagtacaagacactattccttcaaaaagctcagaatagttaaggtaatttaaatcagcaatgacaacaaccccagaattactatgacccacgcagtacaaactgctcaggagtcagaagaaaactgcttttttaaaagggcagtttgggtcatagaacaacagaccatggaaggcatgaccaaaggggagatgacatttgaatctgcaggattaaaagcagcaagggtagcattccaaaaagaaccaccccacaaagatatatgacgtctctatgatttgggtaactgcaattcattccatgtgacttcaggagagaggtcatatttgtgtgtgtagtatgtggaaaatagtgaaaaatgaaaaagctgttaaattgaggaaagtctatccagggaccttatgcatcacattcacgagaacagaattcatcctgtaaaccaggggtgtccaatctttcggcttccctgggccacactgcaagaactgtcttgggccacatataaaggacagctgatgagcaaaaaaaaaaaacagacaacaacaacaaaaaaaacaccccgcaaaaaaaactcctaaaactttaagaaagtttacgaatttgtgttgggtcgcattcaaagctgtcctgggtcccatgcggcccgcgggttagacaacttgctgtaaacagtacaagccagtaatggagtttcacctgtcattttcatgctctatcttcctttaggacaatcatcctaacaagatgtaagatggatcaaaagataacactaaagacagagacagcaatttggaagctatcacacaggcatctgagatcagttactaactggtaagaacagaaatgagaggtatttagaggaagaaaaagggagatgttgcctaacctcagatccaattctctgtaaagcagtagtcaagatcacctggactgtgaagacggtcagggacagaatcccagctaaggaaaaaggataaaatgaaaatcaagataaacatttaagaacgtgaactagggaggaataaaagcactgctgggtaagagtcaagccccagctcaagccttaatttgtggtggaaccaatctgtctggtttcgcgagacaccaggctacccaagatcaagagagggagaaagctagtgctatgtctgaatactagaggagcaagtacaacaaatggaaaatgggatcaagtatgagtgagagttgctaagatgcctggtagggatgcaaaggggtagagagcctggggagagagggtgagggagggaagcactggtttctcaagcaaaagctaaaatttttctattaagatttaacctgatgctacactttggtggtgcagcaagggtctcaaatggtataaaactcaggtgatcatgctttatgtctgtctctagaaaaatgctccaaaaatgataagtagtgataatccgcagtctcgttgcataaaatcagccccaggtgaatgactaagctccatttccctaccccacccttattacaataacctcgacaccaactctagtccgtgggaagataaactaatcggagtcgcccctcaaatcttacagctgctcactcccctgcagggcaacgcccagggaccaagttagccccttaagcctaggcaaaagaatcccgcccataatcgagaagcgactcgacatggaggcgatgacgagatcacgcgaggaggaaaggagggagggcttcttccaggcccagggcggtccttacaagacgggaggcagcagagaactcccataaaggtattgcggcactcccctccccctgcccagaagggtgcggccttctctccacctcctccaccgcagctccctcaggattgcagctcgcgccggtttttggagaacaagcgcctcccacccacaaaccagccggaccgacccccgctcctcccccacccccacgagtgcctgtagcaggtcgggcttgtctcgcccttcaggcggtgggaacccggggcggagccgcggccgccgccatccagaagtctcggccggcagcccgcccccgcctccagcgcgcgcttcctgccacgttgcgcaggggcgcggggccagacactgcggcgctcggcctcggggaggaccgtaccaacgcccgcctccccgccacccccgcgccccgcgcagtggtttcgctcatgtgagactcgagccagtagcaagggcccggtcccacagcttcgacagccaatcaggtgtcgaagacaagcaggcggcgggtaaaccgactcccccgaaggaaggggagggtgggaggacgcccgcgccagagccgatttcactgaccctcccctcccgccgcaggaggccggccgcgcccgcacacccagcatctctacaccccacctacctacccgccccacccagggggcaacgcgagagtcgctaagcggctgcgtactcccgacggcgtaactgacaggagctttactccaaccagaatacgccatttgtgttttcacacacggcgggaggagaaacggccaatcggcgacaagaggctagccggaagcgctcctccctctgcgagagcaatggctccgtccggtttcgagcattttccgctcccttctccctccccctccggttgccgcagggcgggcctccctcccgcctgcatccagccacccctttccctcccaacgtaacaaacattatgttcccgacttcccacgggaaaggcaacccccgcaagccaccagacggcccccctagccacccatccccccagtgtaccgcacctcccctcccaccagagttccgctcccctacctagccgaggctctctgaggagccggagcgccgaagcacagcctcttctctaggcggccccggcggcttccgctgattggcggcgagtgggccaatgggtgcggggcggtgggcggagaggccaatggcgcggcgggagggggcgtgtcccgggtgcccctggcgccggcgctgggaatccccgtgcggtcagtggcgtttccgctcgggcagcgggctgagtgagctgccgccgccgccgccgccgccgccgccgccgccgctgccgggggaggggcggccgccgcccgcctgcgctcagagactcacgcagccccagtcccgccagtccgccaacacagtagtgccggcccccctctttccctggccctgccccccctccccgcctttggctcgctccgcctttctgccccccacccccacctcacgggtacgggccattcccggccaggaaacgccgtggcgccgcgttgggcctaactcgagtcctgccgcctcccgggagtgccgtgcgccgcagcccgggcccaggccccggcagcgcctgggacaaggtaagggtccgacagaaaagagaccgaacctcacgatcgggccccaggggagggaagggtcacctcctccgtctccccgcgctcgctctccttgggtcgtgggcctggccctccccaagctcttaggaggatgctgccacttctcacccccctcgccgccttgcacacaccgttgcaacaccccattttcccagggagagagatccccctctaatctaggcgacccaactccccctttcatgtttttcctgggtcaggacgcttcccctcccccaacgcctcttcaccccctttcctgggaactgcctactccacgtttacctttcccttgaggagaggcctcttgctgccctccgctcgaaatacacaggcatactttttttctctccccgatcccccactccctacccccgttctcgcggccttgtgacagacaactctgatcgctctgggggccgcgatctcccctccgtaatcttcctggacgccttccctctcgttttctggcttcccacctcagatggctgcttcccaaaggcattaccttcgccacccccaccacacgttctctggctccccgtggcgtgtgccacagcgtgtctgagatagcctcgttgaatgtgtagggttcgagcctggagttgagccagattgtgtcgttttacttgccttgggcgtggagaacgatcttgtgagaatatcttcaaaggcagaaaaatattccctttatgaattctctttccctctgcgtgtaagtcgggaatgtgaagaggagtgtaggaaagagccctggttcaagtaggtaaatcgcatgagagggaaagttaaactgttgggaaagccccttctatgctaattgattctatagagtccttgcttgtctcacttcttgggcgtcagtggtctttctcttggatatggatgctgcagtcagctctgctggtctgggtcaggggtgcgtgtatgacctgcattttctgctttctcatgttacttgtgcaatgtattcaccggtaactcatttctttcccagacctctgggttccactgggctttgtctatatttaagttcatttctccagtttccttcctgcacataggtactgaacgaatccccaagttctgtgctaattaccttcatcagttgactaaacaagtttttagatgacatatttgtgaccaaggtcatatttacatttctttgttggacagatgttacatagctatacttgtgattggggaggatccagctgagtggagtgtgctgagctttttaggagagtgtgtactccctatttgaaattattttttggttgttaattttatattattaatgtttttaggtcacagaaagttctaagtggtaattttagatgtgtgggatctgagctaggactaaagcagagaatacccacgtaatcagaggtttctgggctccatagaggacgtagggcttttttttttctattggatttcttccagttttctcaggatcattagttctcttctgtagccaaaaattctggcctgttatgggattagagtctttaaggtttactcagactgtcattatgtgtagaaaaatgaattatgccctttggtaggacatgacacaaggctctgtttctagctgcaaatttaaattagattgtagagtgcttgggaaattggctttcaaaagaccaaagcttaatcttcactcctaaactgctggcttaattaaaatggatatttagaatttggtaaatgttgatttttctaataaaaggccttggtttaaaagggtgaccttaggattgtttctttcttaaaagcataattccagcccttctggcatggagcactggtccaaaaaaaaaaaaaaaaagtgtgtgtaaggagtgggggtggggtaaagagaaggttgttcctttgggttggatcacaggggtgagtatacaaggcagcagcagctgctggctctggagctctggttgctacgtgagaagcttgagtagtgctggctgctgtctccagggaaggacagcagtgcagcgtccattaatgctgctggctgcagggagcagcacttaggcgatggctgcttcaggactaagaagaaaccttgcttttctgggaattttcactgctgagctggtttgctttttattggtggggagatgggaattagtaattcataatctcctacccatttatggatattggcatctggaaactggatcatggttaaagcctttctttttttgtttgtttgatttgatttttgttttttggcagatttttgttttttatctagacatttgtgcttggataggactaaaagttccattagagttttaatttttcaatcagtttaaaaacccaagtaataattttaagaatctttctgataaccacaataggaagaaaataacaggaattttttcctgcagctcacatatcatgccttcctccatctctttaatcatagaatcaattcttattattttgttatgtgtctccatcctttcgattagaccacatttaccttatagacgatttgctaaacattttactaagcttgaactcttaaactctaaaaaggtgccattttggagtggtttctaaataaatatttttaatttgtatattagtaataaacttctccagattagatattttctttggagtttgacttataagattgattcattatatacatgttggatatagccttctgacatcacaaatatatgtctttggccataatccatctgaaatgtaggacagaccagaagaaatatgcagaaatcgaataagtctagttcaggatactgagaagatggcctctgagccccttaggtgatctcccctcccccacaactcctgaacattaggatgatctctgattaagcaaaacagtctgagcgtggaaaaacttgaaggagaaccaccaccaccaattatatgcaatactggacatattcctgtgtgctgtttttcttccccaagactcgtgtatcctatacttttttctctcagaattttgatttgttcattttcgtgtaaatgtacttaaatctcacaaacatctataatttgtagtatcactctggcatttgtggcagagaaccaaaaagaatggaaatgagttttgtcattcacaaatgtggctcacattgttttcccagtaataaaagcagaccaatgaaacagaacctttaatggatactattttaggaggttccaattcttattactatcacatagataagatgcaatagcagataaatatgatttcatgtatactggctgtttgacatacttagggtttaagataaaaatgtttgtagttttttactctgtggcttaagttgctatataaaataattgcttttacactcgaatttcctgttgtttggaaccttttgtgctcttgatattatcattttttagaggatcatacaggcccttttcatagaaggatttacttaagttatacccttgaaaacttttttatatcttttgatactgttttgtgtccaggaactgactttctgaaattattctggcttttctggggagaatgactatttcatttttacctttgaatggggaaataataaagtgcaaagtacagatttgcagataattacttttgctttatcctctccatgttgaaataacttatgaaaaattaggccatagttaacagcagtcaatgactattggatacattttatcagaggggaactggatcatgaataaaataaaattttaaaaataatttttggctgaactctggtgattcatcagtttaatttgaagtcagaaggtctagcagtgaattttatttataaaaattgtatttcaagtgttgaaaactgaaacttcttgaccagtatattttgtttgaggcatcaaactttgcaaaatgtgcatcgtatatttagtgatataactggtagtcatttgtaatttaaagtattctttcaaaggcactctttagaaagtaatgtagtgtacccgtgatgggcagggattggtaccattccttactgccaaaaattccaaaatatgtggcaaaatgattgatttatcttgtgggtgggattctgggaagttcatgaaaggtggagagaatatagtttccttcacttgtctatatacattttgttaaataagtcttaggaaaactgttttattgtatctttaattatgaattgcgtaaaagatacccagtaactttggggggaggtgctgttagaaagcattacattggagagaattcccctaccctgggacaaaatgcattctgtctttaatacttagcgaagggaactatgggataaaataaacaatgaaggtaagctcagtctgctttatatgtgccctcactgagcaaggaatttgtaatcgcatcgtgcctcattcgtttataccatcatattgattttgtttgctgagtacctgagggaataccttacttaatgtaaggtcacattaagtatgtttgatatgaagacagggaaaggaattttctgcttcttggagtaatgtcttagtatttttaaaacacttaagtttttacatcaggccagttttgcctgatgctcatgtctgttgctttggttgggctgctgctttctcttctgtgttcttatgggttcgttgtggtataaggattcccacagctttcatggcagtatgaagtaatgagaagcattgccttagccatgttagttacatgtatacttttggcctatgttatgaatcacaaaaagcggtagctataggaatgtatacaaaatagatttctgtctggggaatcaagtttttgatttgtgctacctaatggaggggaaaatgctgaatttcttgctgctctgtttgagaaatagatggaagcatgggaggagccagagacctctgcagcaggatttggtctaagtagaaaaggaagatttttgtttcaaattgccagctgcttatgtcagactgactcccttattatgcctccagtaggcctgtcaatatggccaaacagctagataagtgcggggcaggacaaagggctctttgcacagcagggaggcaatgttggtgggggaggggcaggaggtaggaaaggcaagaggaggaggttcttttccctgggagattattcagtttggcatacaattaaagaaatcatttttagttcccactcaagcattgaatttttgccaaccacatactattaaccccaaatttgatacatttcagaatatcttgtagggatccattctcgccaaggaaaaataaaaaaataaataaagctctgtataggttaaaataaaataaatcccacactctgcaccctcctaggtgcaagtcacctcccgaggagacccgttctagagctgaattctcattaagaaatggaaaagaatactctatctgaataaaaacacattgtaatacaatgtgtttatttgggttgggattggacctgaacatgtagaataatttgtttccctttatgaaatagttgctcgtagttgtctacaattttatttcattaagataggtagcacattacagctttcatgtgttgggttgccatatgtaaaatgctaactgaagaaaggctactttttaatttcagcctcatccttagttcctggagaacctgatatttcctggagattactccctcccccaccttttagtttaggcaacctcttttgatacatttgtgttcagctcgcatacaagtgggatagttgcatccagtttattaagacttagtatgaatcatagagttggaaaagatctgttggttatctggtcctttaaaccaaaatcataatgaaatattttgaaatttgggtccctattgaagttttcattaaaatgttaaaggatcggtgttctgaacaacatttttagttacttttaaaataaatgttttgcgtcagttctttttttaaaaataaagaatttcatttataggcaaattagctggcaattatttgaattgtgataggatttctcttttatgaaggaatatatgacaaggtttttcaaaatgcttaatatattttaaaagactttaatttttagaaataattggtttgaacagttttccaagagcacatttgttgcttgggttgaggtaccacctatattgcaatgttactaaactagccttaaagttttcccttctgtctatactgcatgcaacaataaagggaactggaatgttaatttccatttatggattagcagaggagatgttttaaccgattaataaccaaaaaactgcctttcgtacacgtaatattaagcaagcctgaccaagttttgtgttatttctctctgttaaagaaaactggatgtgttactacttaacattatattgttatttaatggtcttggcagtaatgatataatatttcgaccaaaagaaattttgagtaattaattattattgtaattagttggaagtttctcatcagtaaaatagcaacagcattaacacaaaatctagtgagctatattttatattactacagaaatttagggtagtcatttctttctttataatttattcacatggattatttccataaatttgtgggactaaaatagaagccatctagtcaagcaccagtctccataccagacagttttctctgcatgtgctatgacccacattgccagtattaaacatcctttacaccctcccccttcccagataattagaaatctcttcagggtagcttccattgctcctattacctggatcttgctagaggctctaagaagttcctggtaaaagtgagacagtaagggaccacattttgattccaaaggttttgataactgttagggctccccaaacagctaatctcattttcaccaagacttagccagcagagggctggaatggaggtgaaacacaagcactgtacctcatcttgcctgtgcagctgctccaccttatttcctgctattattatctcacaacgcctcctcccatcaaaaagaaactaggacaaagggggaaaattggatgggctaatgtgatttttattatgctaggttgtgggcttgtttatatgtacttaaatacaaagctaatttgccccattcttaaaagtctttagtgatagagattttgtaacttctgtatcttctactttctttcttgataaaccatttcagattctcagccttacagaaagaaaggttttaagcatacttaattttcgttggccgttcacagtcattattaccaccagatgccactgtattattagcttgaagaaaggtgggctctcttctgtacataatatctgcaatttgttttggaaaatactaatttgtataaatctgatttatgactaaaataaggttaaaaattagacctctatgtatgtttaccctattaccttagtgggggtgaaattaattagctctttgaacataaatttttcatgtcttagagttcttttttcaagctgcataatttatgttcttcaagccatttttatcccataccacccccacaaagggggaaattttattttttatcatttttattgtctttcaatggtgagattttcgccaccccactcctgaaatgtgaagactcaaataaaactgagtaatctaataaggtatatgcgttgctgaatgtagtaagatgattgtttcatcattcttagatattatgatctagtttgaatctggtttccagtatcatgttagcatatttaatactgttgatatgttaattttaatacatgcccaggtggatctccttgctttctatttgtgccccttgtttgtcgttttgtatgaagggggtttttgttgttggattttcttccccatctctgtgtcctgttatgttctttggcttatgtttcaaaaattctgtttcctaccaccaacctctgtacatgccacaacacatacaatttgtactttcacagtttctgtgaagtaggatgatctgcagttaataatcaactgtttgggcattcttggtatccaaggaaggttttacttagaaggaagaacctggaaggacctgttggcaattagactacttctgcgtttattttacattttcccttattaacgtaggctgttgagagttgacttgttttataagagaaaccagattgacagagaagacccccaatcagatagagttattttaaaaataaatgtgtttattatggtaacatttggggtagaatctaaagggcatatttttaaaaaaacttttagttctaaagacaaaagagtttaacctaaaacagaacaaagagaagggcctttgaagcagtatgattgattatat

SEQ ID NO: 5 - DNA, 6473 bp, Cricetulus griseus
  source 1..6473: mol_type = genomic DNA
tctagaaacaaaaccaaaaatattaagtcaggcttggcttcaggtgctggggtggagtgctgacaaaaatacacaaattcctggctttctaaggctttttcggggattcaggtattgggtgatggtagaataaaaatctgaaacataggtgatgtatctgccatactgcatgggtgtgtatgtgtgtgtatgtgtgtctgtgtgtgtgcccagacagaaataccatgaaggaaaaaaacacttcaaagacaggagagaagagtgacctgggaaggactccccaatgagatgagaactgagcacatgccagaggaggtgaggactgaaccattcaacacaagtggtgaatagtcctgcagacacagagagggccagaagcactcagaactccagggggtcaggagtggttctctggaggcttctgcccttggaggttcctgaggaggaggcttccatattgaaaatgtagttagtggccgtttccattagtacagtgactagagagagctgagggaccactggactgaggcctagatgctcagtcagatggccatgaaagcctagacaagcacttccgggtggaaaggaaacagcaggtgtgaggggtcaggggcaagttagtgggagaggtcttccagatgaagtagcaggaacggagacgcactggatggccccacttgtcaaccagcaaaagcttggatcttgttctaagaggccagggacatgacaagggtgatctcggtttttaaaaggctttgtgttacctaatcacttctattagtcagatactttgtaacacaaatgagtacttggcctgtattttagaaacttctgggatcctgaaaaaacacaatgacattctggctgcaacacctggagactcccagccaggccctggacccgggtccattcatgcaaatactcagggacagattcttcactaggtactgatgagctgtcttggatgcaaatgtggcctcttcattttactacaagtcaccatgagtcaggaggtgctgtttgcacagtgtgactaagtgatggagtgttgactgcagccattcccggccccagcttgtgagagagatccttttaaattgaaagtaagctcaaagttaccacgaagccacacatgtataaactgtgtgaataatctgtgcacatacacaaaccatgtgaataatctgtgtacatgtataaactgtgtgaataatctgtgtgcagcctttccttacctactaccttccagtgatcaggtttggactgcctgtgtgctactggaccctgaatgtccccaccgctgtcccctgtcttttacgattctgacatttttaataaattcagcggcttcccctctgctctgtgcctagctataccttggtactctgcattttggtttctgtgacatttctctgtgactctgctacattctcagatgacatgtgacacagaaggtgttccctctggagacatgtgatgtccctgtcattagtggaatcagatgcccccaaactgttgtccagtgtttgggaaagtgacacgtgaaggaggatcaggaaaagaggggtggaaatcaagatgtgtctgagtatctcatgtccctgagtggtccaggctgctgacttcactcccccaagtgagggaggccatggtgagtacacacacctcacacatactatatccaacacacacacacacacacacacacacacgcacgcacgcacgcacgcacgcacacatgcacacacacgaactacatttcacaaaccacatacgcatattacaccccaaacgtatcacctatacataccacacatacacacccctccacacatcacacacataccacacccacacacagcacacacatacataggcacacattcacacaccacacatatacatttgtgtatgcatacatgcatacacacacaggcacacagacaccacacacatgcattgtgtacgcacacatgcatacacacacataggcacacattgagcacacacatacatttgtgtacgcacactacatagacatatatgcatttgtatatgcacacatgcatgcacacatacataggcacacatagagcacacacatacatttgtgtatgcacacatgcacacaccaatcacatgggaagactcaggttcttcactaaggttcacatgaacttagcagttcctggttatctcgtgaaacttggaagattgctgtggagaagaggaagcgttggcttgagccctggcagcaattaaccccgcccagaagaagtaggtttaaaaatgagagggtctcaatgtggaacccgcagggcgccagttcagagaagagacctacccaagccaactgagagcaaaggcagagggatgaacctgggatgtagtttgaacctctgtaccagctgggcttcatgctattttgttatatctttattaaatattcttttagttttatgtgcgtgaataccttgcttgcataaatgtatgggcactgtatgtgttcttggtgccggtggaggccaggagagggcatggatcctccggagctggcgtttgagacagttgtgacccacagtgtggggtctgggaactgggtcttagtgttccgcaagtgcagctggggctcttaacctctgagccatccctccagcttcaagaaacttattttcttaggacatgggggaagggatccagggctttaggcttgtttgttcagcaaatactcttttcgtgtattttgaattttattttattttacttttttgggatagaatcacattctgcagctcaggctgggcctgaactcatcaaaatcctcctgtctcagtctaccaggtgataagattactgatgtgagcctggctttgacaagcactttagagtccccagcccttctggacacttgttccaagtataatatatatatatatatatatatatatatatatatatatatatattgtgtgtgtgtgtttgtgtgtgtatgagacacttgctctaagggtatcatatatatccttgatttgcttttaatttattttttaattaaaaatgattagctacatgtcacctgtatgcgtctgtatcatctatatatccttccttccttctctctctttctctcttcttcttctcacccccaagcatctattttcaaatccttgtgccgaggagatgccaagagtctcgttgggggagatggtgagggggcgatacaggggaagagcaggaggaaagggggacagactggtgtgggtctttggagagctcaggagaatagcagcgatcttccctgtccctggtgtcacctcttacagccaacaccattttgtggcctggcagaagagttgtcaagctggtcgcaggtctgccacacaaccccaatctggccccaagaaaaggcacctgtgtgtgactctggggttaaaggcgctgcctggtcgtctccagctggacttgaaactcccgtttaataaagagttctgcaaaataatacccgcagagtcacagtgccaggttcccgtgctttcctgaagcgccaggcacgggttccctaggaaatggggccttgcttgccaagctcccacggcttgccctgcaaacggcctgaatgatctggcactctgcgttgccactgggatgaaatggaaaaaagaaaaagaagaagtgtctctggaagcgggcgcgctcacacaaacccgcaacgattgtgtaaacactctccattgagaatctggagtgcggttgccctctactggggagctgaagacagctagtgggggcggggggaggaccgtgctagcatccttccacggtgctcgctggctgtggtgcatgccgggaaccgaaacgcggaactaaagtcaagtcttgctttggtggaactgacaatcaacgaaatcacttcgattgttttcctctttttactggaattcttggatttgatagatgggggaggatcagagggggaggggaggggcggggagacggagggaggaggggaggaggggaggaggggaggaggggaggaggggaagggatggaggaaaatactaacttttctaattcaacatgacaaagattcggagaaagtgcaccgctagtgaccgggaggaggaatgccctattgggcattatattccctgtcgtctaatggaatcaaactcttggttccagcaccaaggattctgagcctatcctattcaagacagtaactacagcccacacggaagaggctatacaactgaagaaataaaattttcactttatttcatttctgtgactgcatgttcacatgtagagagccacctgtgtctaggggctgatgtgctgggcagtagagttctgagcccgttaactggaacaacccagaactcccaccacagttagagcttgctgagagagggaggcccttggtgagatttctttgtgtatttatttagagacagggtctcatactgtagtccaagctagcctccagctcacagaaattctcctgttccggtttccaaagtactggagttatgagtgtgtgttaattgaacgctaagaatttgctgattgaagaaaacctcaagtgggtttggctaatccccacgaccccagaggctgaggcaggaggaatgagagaattcaaggtttgccagagccacagggtgagctcaatgtggagactgtgagggtgagctcaatgtggagactgtgagggtgagctcaatgtggagactgtgagggtgagctcaatgtggagactgtgagggtgagctcaatgtggagactgtgagggtgagctcaatgtggagacctgtatcaagataataatagtagtagtaacaatgcaggcgagggtgtggttgagtggtagagcagttagttgatttgacatgcttgaggtctcccggtccatctgtggccctgcaacaggaagggagggaggaagggggggaacgagagagaggaaagagagacagaagctaagatagggaatgagagaggaaggaagaaacgggaagaaattcagactccttcctgagttccgccaacgcctagtgacatcctgtgcacaccctaaggtggcctttgtgtggcactggcttgggtggtcgggaaaggcattttcagcttgttgcagaactgccacagtagcatgctgggtccgtgaaagtttctgcccgttaacaagaagtctctactacttgtgacctcaccagtgaaaatttctttaattgtctcctggtgttctgggttttgcatttttgtttctaaggatacattcctgggtgatgtcatgaagtccccaaagacacagtggggctgtgttggattgggaaagatgatttatctggggtgtcaaaaggaaaagaagggaaacaggcacttgggaaaatgtcctcccgcccacccgaattttggcttggcaaccgtggtggaggagcaagaaacacgtggacgtttgaggaggcatggggtcctaggaggacaggaagcagaaggagagagctgggctgacagcctgcaggcattgcacagtttcagaaggagattacagcatgactgagtttttagggatccaacagggacctgggtagagattctgtgggctctgaggcaacttgacctcagccagatggtatttgaataacctgctcttagagggaaaacagacatagcaaacagagccacgtttagtgatgaaactctcactttgcctgagtcatgtgcggccatgcccaggggtcaggctgacactcaactcaaaaacaagtgagaaattgaagacaatccgtggtggcagctactggaagggccaccacatccccagaaagagtggagctgctaaaaagccatttgtgataggcacagttatcttgaatgcatggagcagagattacggaaaaatcgagaatgttaatgaggcaacattcgagttgagtcattcagtgtgggaaacccagacgcttccatcccctaaaaggaacatcttgctctcagtcaaaatggaaataaaaattggggcttgaatttggcaaatgattcagaactctgtgtaggtattttcacacgcacagtggataattttcatgttggagtttatttgtgctaaaaggcagaaaagggtaaaaagcacatcttaagagttatgaggttctacgaataaaaataatgttacttacagctattccttaattagtacccccttccacctgtggtaatttcctgagatagtcagtggggaaaagatctctccttctcttctttctccccctcccctcctctccctccctccctccctccctccctcctctccctccctccccctttccttctttctttgctccttctcctctgcctccttctccctttcttcttcatttattctaagtagcttttaacagcacaccaattacctgtgtataacgggaaaacacaggctcaagcagcttagagaagattgatctgtgttcact

SEQ ID NO: 6 - DNA, 7045 bp, Cricetulus griseus
  source 1..7045: mol_type = genomic DNA
actagcgtgcaattcagaggtgggtgaagataaaaggcaaacatttgaggccatttccttatttggcacggcacttaggaagtggaacatgcctaatctactggtttgtaccacctttccctataatggactgtttgggaagctcctgggcaaccgattctggcatctcattggtcagaggcctgttaaatggtactcttatttgcaaagaaggctgtaacttgtagctttaaaagcctctcctcaagaaagaagggagaaaggatatggctagacatatctaatagacttaaccactgtgaaaagccttagtatgaatcagatagaacctatttttaactcagttttgaaaaaaataatctttatatttatttgtgtgtgtgtgtgtgtgtgtgtgtgtgtgtgtgtgtgtgtgtgtgtgaaccacatgtagcaggtgctggaggaggccagaagagggcaccagatctcctggaactgacaccacacatggttatgagctgcctgatgtgggtgctgggaactgaactctcgtgttctgcaagagcagcaactgttctcttaactgatgagccatctctccagccccccccataattttaattgttcattttagtaaattttattcataatcaattatcacagtataaaacaatgattttatatatatcatatacatatcaaggatgacagtgagggggatatgtgtgtgtgtgtgtgtgtgtgtgtgtgtgtgtgtgtgtgttatttgtgtgtgtgctttttaagaaggtgccatagtcactgcatttctctgaaggatttcaaaggaatgagacatgtctgtctgccaggaaccctatcttcctctttgggaatctgacccaaatgaggtattctgaggaactgaatgaagagctcaagtagcagtgtcttaaacccaaatgtgctgtctagagaaagtcaacgtcatcagtgagctgaggagagatttactgagcggaagacaagcgctctttgatttaagtggctcgaacagtcacggctgtggagtggagcctgtgctcaggtctgaggcagtctttgctagccagctgtgatgagcagtgaagaaagggtggagatggaggcagggtgggagcagggctatggttcagactaggtatcgtgagcacaccagctggttgacttgtggtctgtgggtcaggcgttgtaaacgccctcagggtcaggcagtcacattgcttgaagctgaatgggtgaggcaacacagagagtgcaaagaaggcaaagtaccacctcttccccgacccaggtcacttctgggttatagctgagactccggacagcatgcaaccagctggttagagcttcagggaaaacttgatgtctgcatgttgctatgaaatgtgattcggtacatctggagaaaatttataatgctggctcagtcaagcactgaacaaaggtaccttggctttgggagctacatgacattgacttgtaggcagacttttttttttctgcccgccaattcccagataaccaatatggaggctcaatattaattataaatgctcggctgatagctcaggcttgttactagctaactcttccaacttaaatgaacccatttctattatctacattctgccacgtgactttaccttgtacttcctgtttcctctccttgtctgactctgcccttctgcttcccagagtccttagtctggttctcctgcctaaccttatcctgcccagctgctgaccaagcatttataattaatattaagtctcccagtgagactctcatccagggaggacttgggtgctcccccctcctcattgccatccgtgtcttcctcttccctcgcttccccctcctcttcctgctcttcctcctccacccctcctttcatagtattgatggcaagggtgttctagaatggaggagtgcccataggcatgcaaagaaaccagttaggatgctctgtgaggggttgtaatcataagcgatggacacaattcaagccacagagtgaagacggaaggatgcactgtgctctagagcaacttctggggcagaatcacagggtgagtttctgacttgagggcgaagaggccacgaggaagggagtgagtttgtctgagctagaagctacggcccacctcttggtagcagacctgcccacaagcatgctttgttaatcatgtgggatctgattttcctctaaatctatgttcaactcttaagaaaatgtgaattctcacattaaaatttagatatacgtcttttggtggggggggtgtaaaaaatcctcaagaatatggatttctgggggccggagagatggctcagaggttaagagaactggttgctcttctagacattctgagttcaattcccagcaaccacatggtggctcacaaccatctgtaatgcgacctggtgccatcttctgacatgcatggatacatgcaggcagaaagctgtatacatagtaaattgataaatctttttttaaaaagagtatggattctgccgggtgttggtggcgcacgcctttaatcccagcactctggaggcagaggcaggtggatctctgtgagttcgagaccagcctggtctataagagctagttccaggacagcctccaaagccacagagaaaccctgtctcgaaaaaccaaaaaaaaaaaaaaaaaaaaaaaaaaaaaaagagtatggattctaagaaagccgtaacagctggagctgtgtacggagttcagcgtggtactagaagaacagacattcatgatgaaacaccccaggatttttacttagtatctagtttccattgttgttttgagaccggctcttatgctctccaggctggcctcaaactgctgatcttcccgcctctacctctcaagtcctgggactacttggctcataaaacagtttttgtcgggctccctgaagttatggttgtacaaaccgtgggggtcaatatactcacttgggcagagagagaaggtctgaatcccagacaatgactgcatctcaggacagttgggaagaggacaatggcagaaggacttagaaaagatagactggagggtggaaaagcagcaggaacagagaaacaaaacaggaagcttgctatccagggccactctggagtcctgtggcaagatggaagcgggctaggggaatacatttgtgctactgtgtgtgtgtgtgtgtgtgtgtgtgtgtgtgtgtgtgatcaatgcctatcaatgttgaaggggaaatatgtataccacattgattctgggagcaattctcagtatctggcctagagaaaggaatggcccctgcagaatagacagagtgaatggtgccctttatcatttgctaaagtgaaggagaaataaacatccttccatagagtttcaggtaaatgaaccccacagttcatctgtgccgtggtggaggcctggccaacagttaaaaagattagacacggacaaagtctgaaggaaacacctcgaataggaagaggagagccacctcattctgtaactttcctcaaggggaagatgttccaagagtgggaataaatggtcaaaggggggatttttaattaggaaaacgatttcctgtatcacttgtgaaactggaggttgatttggggcataggacaatagatttgatgctttgcaaaaagctgtttcaaagcagagaaatggaatagagacaattatgtagcgaggagggagggtggggcgaagatggagacagagaagtggaagctgactttagggaagaggaacatagaccacaggggcggggcggggggcaggggcggggggcggggctcaaaggaggcagtgggaacgttgctagtgttcgcagcgtaagcgtgaatgtgcaagcgtctttgtggtgtgtgaccaggagtagcgtggctggcttgtgtgctgcttgtaatcccagtctttgaggtttccacactgttccacagtgggtgtgattttccctcggagagcatgagggctctgctttccccacatcctccccagcgttcgttggtatttgtttccaagatgttagtgggtgagacaaagcctctctgttgatttgcctttaacaggtgacaaaaaaagctcaaccaggagacatttttgccttcttggaaggtaatgctcccatgtagagcaatgggacccatctctaaggtgaggctactcttgcagtttgcacccagctcttctgatgcaggaaggaagttggtgggcaagcaagactgtttgcttcttgcgatggacacattctgcacacaaaggctcaggaggggagaaggctgtttgatgtttagcactcaggaaggcccctgatgcatctgtgattagctgtctccatctgtggagcagacacggactaactaaaaaccagtgtttttaaattgtcaagcctttaaggtgaggaaattgacttattgtgctgggccatacgtagagcaagtgctctgcattgggccaacccccggctctggtttctaggcaccagaatggcctagaactaactcacaatcctcccattccaggtctcaggtgctagaatgaaccactataccagcctgcctgcctgcctacctgccttcctaaattttaaatcatggggagtaggggagaatacacttatcttagttagggtttctattgctgtgaagagacaccatgagcatggcaactcttataaaggaaaacatttagttgggtggcagtttcagaggttttagtacattgtcatcatggctgggaacatgatggcatgcagacagacatggtgctggagaaagggatgagagtcctacatcttgcaggcaacaggacctcagctgagacactggctggtaccctgagcataggaaacctcacagcccaccctcacagtgacatatttccttcaacaaagccatacctcctaatagtgccactccctatgagatgacagggccaattacattcaaactgctataacactttaaagtattttatttttattattgtaaattatgtatgtagctgggtggtggcagccgaggtgcacgcctttaatcccagcacttgggaggcagaggcagatggatctctgtgagttcaagaccagcctggtctataagagctagttgcaaggaaggatatacaaagaacagttctaggatagccttcaaagccacagagaagtgctgtcttgaaaaccaaaaattgtgctgggacctgtctctgctttggttgcttcccactcccccagagctggactcttggtcaacactgaatcagctgcaaaataaactcctggattcctctcttgtaacaggagcccgaagtcaggcgcccacttgtcttctcgcaggattgccatagactttttctgtgtgcccaccattccagactgaagtagagatggcagtggcagagactgggaaggctgcaacgaaaacaggaagttattgcaccctgggaatagtctggaaatgaagcttcaaaacttgcttcatgttcagttgtacacagactcactcccaggttgactcacacgtgtaaatattcctgactatgtctgcactgcttttatctgatgcttccttcccaaaatgccaagtgtacaaggtgagggaatcacccttggattcagagcccagggtcgtcctccttaacctggacttgtctttctccggcagcctctgacacccctccccccattttctctatcagaaggtctgagcagagttggggcacgctcatgtcctgatacactccttgtcttcctgaagatctaacttctgacccagaaagatggctaaggtggtgaagtgtttgacatgaagacttggtcttaagaactggagcaggggaaaaaagtcggatgtggcagcatgtacccgaaatcccagaactggggaggtagagacggatgagtgcccggggctagctggctgctcagccagcctagctgaattgccaaattccaactcctattgaaaaacctttaccaaacaaacaaacaaacaaataataacaacaacaacaacaacaaactaccccatacaaggtgggcggctcttggctcttgaggaatgactcacccaaacccaaagcttgccacagctgttctctggcctaaatggggtgggggtggggcagagacagagacagagagagacatgacttcctgggctgggctgtgtgctctaggccaccaggaactttcctgtcttgctctctgtctggcacagccagagcaccagcacccagcaggtgcacacacctccctccgtgcttcttgagcaaacacaggtgccttggtctgtctattgaaccggagtaagttcttgcagatgtatgcatggaaacaacattgtcctggttttatttctactgttgtgataaaaaccggggaactccaggaagcagctgaggcagaggcaaatgcaaggaatgctgcctcctagcttgctccccatggcttgccgggcctgctttctgcaagcccttctctccccattggcatgcctgacatgaacagcgtttgaaatgctctcaaatgtcactttcaaagaaggcttctctgatcttgctaactaaatcagaccatgtttcaccgtgcattatctttctgctgtctgtctgtctgtctgtctgtctatctgtctatcatctatcaatcatctatctatctatcttctatttatctacctatcattcaatcatctatcttctaactagttatcatttatttatttgtttacttactttttttatttgagacagtatttctctgagtgacagccttggctgtcctggaacccattctgtaaccaggctgtcctcaaactcacagagatccaactgcctctgcctctctggtgctggggttaaagacgtgcaccaccaacgccccgctctatcatctatttatgtacttattattcagtcattatctatcctctaactatccatcatctgtctatccatcatctatctatctatctatctatctatctatctatctatcatccatctataatcaattg

SEQ ID NO: 7 - DNA, 6473 bp, Cricetulus griseus
  source 1..6473: mol_type = genomic DNA
agtgaacacagatcaatcttctctaagctgcttgagcctgtgttttcccgttatacacaggtaattggtgtgctgttaaaagctacttagaataaatgaagaagaaagggagaaggaggcagaggagaaggagcaaagaaagaaggaaagggggagggagggagaggagggagggagggagggagggagggagaggaggggagggggagaaagaagagaaggagagatcttttccccactgactatctcaggaaattaccacaggtggaagggggtactaattaaggaatagctgtaagtaacattatttttattcgtagaacctcataactcttaagatgtgctttttacccttttctgccttttagcacaaataaactccaacatgaaaattatccactgtgcgtgtgaaaatacctacacagagttctgaatcatttgccaaattcaagccccaatttttatttccattttgactgagagcaagatgttccttttaggggatggaagcgtctgggtttcccacactgaatgactcaactcgaatgttgcctcattaacattctcgatttttccgtaatctctgctccatgcattcaagataactgtgcctatcacaaatggctttttagcagctccactctttctggggatgtggtggcccttccagtagctgccaccacggattgtcttcaatttctcacttgtttttgagttgagtgtcagcctgacccctgggcatggccgcacatgactcaggcaaagtgagagtttcatcactaaacgtggctctgtttgctatgtctgttttccctctaagagcaggttattcaaataccatctggctgaggtcaagttgcctcagagcccacagaatctctacccaggtccctgttggatccctaaaaactcagtcatgctgtaatctccttctgaaactgtgcaatgcctgcaggctgtcagcccagctctctccttctgcttcctgtcctcctaggaccccatgcctcctcaaacgtccacgtgtttcttgctcctccaccacggttgccaagccaaaattcgggtgggcgggaggacattttcccaagtgcctgtttcccttcttttccttttgacaccccagataaatcatctttcccaatccaacacagccccactgtgtctttggggacttcatgacatcacccaggaatgtatccttagaaacaaaaatgcaaaacccagaacaccaggagacaattaaagaaattttcactggtgaggtcacaagtagtagagacttcttgttaacgggcagaaactttcacggacccagcatgctactgtggcagttctgcaacaagctgaaaatgcctttcccgaccacccaagccagtgccacacaaaggccaccttagggtgtgcacaggatgtcactaggcgttggcggaactcaggaaggagtctgaatttcttcccgtttcttccttcctctctcattccctatcttagcttctgtctctctttcctctctctcgttcccccccttcctccctcccttcctgttgcagggccacagatggaccgggagacctcaagcatgtcaaatcaactaactgctctaccactcaaccacaccctcgcctgcattgttactactactattattatcttgatacaggtctccacattgagctcaccctcacagtctccacattgagctcaccctcacagtctccacattgagctcaccctcacagtctccacattgagctcaccctcacagtctccacattgagctcaccctcacagtctccacattgagctcaccctgtggctctggcaaaccttgaattctctcattcctcctgcctcagcctctggggtcgtggggattagccaaacccacttgaggttttcttcaatcagcaaattcttagcgttcaattaacacacactcataactccagtactttggaaaccggaacaggagaatttctgtgagctggaggctagcttggactacagtatgagaccctgtctctaaataaatacacaaagaaatctcaccaagggcctccctctctcagcaagctctaactgtggtgggagttctgggttgttccagttaacgggctcagaactctactgcccagcacatcagcccctagacacaggtggctctctacatgtgaacatgcagtcacagaaatgaaataaagtgaaaattttatttcttcagttgtatagcctcttccgtgtgggctgtagttactgtcttgaataggataggctcagaatccttggtgctggaaccaagagtttgattccattagacgacagggaatataatgcccaatagggcattcctcctcccggtcactagcggtgcactttctccgaatctttgtcatgttgaattagaaaagttagtattttcctccatcccttcccctcctcccctcctcccctcctcccctcctcccctcctccctccgtctccccgcccctcccctccccctctgatcctcccccatctatcaaatccaagaattccagtaaaaagaggaaaacaatcgaagtgatttcgttgattgtcagttccaccaaagcaagacttgactttagttccgcgtttcggttcccggcatgcaccacagccagcgagcaccgtggaaggatgctagcacggtcctccccccgcccccactagctgtcttcagctccccagtagagggcaaccgcactccagattctcaatggagagtgtttacacaatcgttgcgggtttgtgtgagcgcgcccgcttccagagacacttcttctttttcttttttccatttcatcccagtggcaacgcagagtgccagatcattcaggccgtttgcagggcaagccgtgggagcttggcaagcaaggccccatttcctagggaacccgtgcctggcgcttcaggaaagcacgggaacctggcactgtgactctgcgggtattattttgcagaactctttattaaacgggagtttcaagtccagctggagacgaccaggcagcgcctttaaccccagagtcacacacaggtgccttttcttggggccagattggggttgtgtggcagacctgcgaccagcttgacaactcttctgccaggccacaaaatggtgttggctgtaagaggtgacaccagggacagggaagatcgctgctattctcctgagctctccaaagacccacaccagtctgtccccctttcctcctgctcttcccctgtatcgccccctcaccatctcccccaacgagactcttggcatctcctcggcacaaggatttgaaaatagatgcttgggggtgagaagaagaagagagaaagagagagaaggaaggaaggatatatagatgatacagacgcatacaggtgacatgtagctaatcatttttaattaaaaaataaattaaaagcaaatcaaggatatatatgatacccttagagcaagtgtctcatacacacacaaacacacacacacaatatatatatatatatatatatatatatatatatatatatatattatacttggaacaagtgtccagaagggctggggactctaaagtgcttgtcaaagccaggctcacatcagtaatcttatcacctggtagactgagacaggaggattttgatgagttcaggcccagcctgagctgcagaatgtgattctatcccaaaaaagtaaaataaaataaaattcaaaatacacgaaaagagtatttgctgaacaaacaagcctaaagccctggatcccttcccccatgtcctaagaaaataagtttcttgaagctggagggatggctcagaggttaagagccccagctgcacttgcggaacactaagacccagttcccagaccccacactgtgggtcacaactgtctcaaacgccagctccggaggatccatgccctctcctggcctccaccggcaccaagaacacatacagtgcccatacatttatgcaagcaaggtattcacgcacataaaactaaaagaatatttaataaagatataacaaaatagcatgaagcccagctggtacagaggttcaaactacatcccaggttcatccctctgcctttgctctcagttggcttgggtaggtctcttctctgaactggcgccctgcgggttccacattgagaccctctcatttttaaacctacttcttctgggcggggttaattgctgccagggctcaagccaacgcttcctcttctccacagcaatcttccaagtttcacgagataaccaggaactgctaagttcatgtgaaccttagtgaagaacctgagtcttcccatgtgattggtgtgtgcatgtgtgcatacacaaatgtatgtgtgtgctctatgtgtgcctatgtatgtgtgcatgcatgtgtgcatatacaaatgcatatatgtctatgtagtgtgcgtacacaaatgtatgtgtgtgctcaatgtgtgcctatgtgtgtgtatgcatgtgtgcgtacacaatgcatgtgtgtggtgtctgtgtgcctgtgtgtgtatgcatgtatgcatacacaaatgtatatgtgtggtgtgtgaatgtgtgcctatgtatgtgtgtgctgtgtgtgggtgtggtatgtgtgtgatgtgtggaggggtgtgtatgtgtggtatgtataggtgatacgtttggggtgtaatatgcgtatgtggtttgtgaaatgtagttcgtgtgtgtgcatgtgtgcgtgcgtgcgtgcgtgcgtgcgtgtgtgtgtgtgtgtgtgtgtgtgtgttggatatagtatgtgtgaggtgtgtgtactcaccatggcctccctcacttgggggagtgaagtcagcagcctggaccactcagggacatgagatactcagacacatcttgatttccacccctcttttcctgatcctccttcacgtgtcactttcccaaacactggacaacagtttgggggcatctgattccactaatgacagggacatcacatgtctccagagggaacaccttctgtgtcacatgtcatctgagaatgtagcagagtcacagagaaatgtcacagaaaccaaaatgcagagtaccaaggtatagctaggcacagagcagaggggaagccgctgaatttattaaaaatgtcagaatcgtaaaagacaggggacagcggtggggacattcagggtccagtagcacacaggcagtccaaacctgatcactggaaggtagtaggtaaggaaaggctgcacacagattattcacacagtttatacatgtacacagattattcacatggtttgtgtatgtgcacagattattcacacagtttatacatgtgtggcttcgtggtaactttgagcttactttcaatttaaaaggatctctctcacaagctggggccgggaatggctgcagtcaacactccatcacttagtcacactgtgcaaacagcacctcctgactcatggtgacttgtagtaaaatgaagaggccacatttgcatccaagacagctcatcagtacctagtgaagaatctgtccctgagtatttgcatgaatggacccgggtccagggcctggctgggagtctccaggtgttgcagccagaatgtcattgtgttttttcaggatcccagaagtttctaaaatacaggccaagtactcatttgtgttacaaagtatctgactaatagaagtgattaggtaacacaaagccttttaaaaaccgagatcacccttgtcatgtccctggcctcttagaacaagatccaagcttttgctggttgacaagtggggccatccagtgcgtctccgttcctgctacttcatctggaagacctctcccactaacttgcccctgacccctcacacctgctgtttcctttccacccggaagtgcttgtctaggctttcatggccatctgactgagcatctaggcctcagtccagtggtccctcagctctctctagtcactgtactaatggaaacggccactaactacattttcaatatggaagcctcctcctcaggaacctccaagggcagaagcctccagagaaccactcctgaccccctggagttctgagtgcttctggccctctctgtgtctgcaggactattcaccacttgtgttgaatggttcagtcctcacctcctctggcatgtgctcagttctcatctcattggggagtccttcccaggtcactcttctctcctgtctttgaagtgtttttttccttcatggtatttctgtctgggcacacacacagacacacatacacacacatacacacccatgcagtatggcagatacatcacctatgtttcagatttttattctaccatcacccaatacctgaatccccgaaaaagccttagaaagccaggaatttgtgtatttttgtcagcactccaccccagcacctgaagccaagcctgacttaatatttttggttttgtttctaga

SEQ ID NO: 8 - DNA, 7045 bp, Cricetulus griseus
  source 1..7045: mol_type = genomic DNA
caattgattatagatggatgatagatagatagatagatagatagatagatagatagatgatggatagacagatgatggatagttagaggatagataatgactgaataataagtacataaatagatgatagagcggggcgttggtggtgcacgtctttaaccccagcaccagagaggcagaggcagttggatctctgtgagtttgaggacagcctggttacagaatgggttccaggacagccaaggctgtcactcagagaaatactgtctcaaataaaaaaagtaagtaaacaaataaataaatgataactagttagaagatagatgattgaatgataggtagataaatagaagatagatagatagatgattgatagatgatagacagatagacagacagacagacagacagacagcagaaagataatgcacggtgaaacatggtctgatttagttagcaagatcagagaagccttctttgaaagtgacatttgagagcatttcaaacgctgttcatgtcaggcatgccaatggggagagaagggcttgcagaaagcaggcccggcaagccatggggagcaagctaggaggcagcattccttgcatttgcctctgcctcagctgcttcctggagttccccggtttttatcacaacagtagaaataaaaccaggacaatgttgtttccatgcatacatctgcaagaacttactccggttcaatagacagaccaaggcacctgtgtttgctcaagaagcacggagggaggtgtgtgcacctgctgggtgctggtgctctggctgtgccagacagagagcaagacaggaaagttcctggtggcctagagcacacagcccagcccaggaagtcatgtctctctctgtctctgtctctgccccacccccaccccatttaggccagagaacagctgtggcaagctttgggtttgggtgagtcattcctcaagagccaagagccgcccaccttgtatggggtagtttgttgttgttgttgttgttattatttgtttgtttgtttgtttggtaaaggtttttcaataggagttggaatttggcaattcagctaggctggctgagcagccagctagccccgggcactcatccgtctctacctccccagttctgggatttcgggtacatgctgccacatccgacttttttcccctgctccagttcttaagaccaagtcttcatgtcaaacacttcaccaccttagccatctttctgggtcagaagttagatcttcaggaagacaaggagtgtatcaggacatgagcgtgccccaactctgctcagaccttctgatagagaaaatggggggaggggtgtcagaggctgccggagaaagacaagtccaggttaaggaggacgaccctgggctctgaatccaagggtgattccctcaccttgtacacttggcattttgggaaggaagcatcagataaaagcagtgcagacatagtcaggaatatttacacgtgtgagtcaacctgggagtgagtctgtgtacaactgaacatgaagcaagttttgaagcttcatttccagactattcccagggtgcaataacttcctgttttcgttgcagccttcccagtctctgccactgccatctctacttcagtctggaatggtgggcacacagaaaaagtctatggcaatcctgcgagaagacaagtgggcgcctgacttcgggctcctgttacaagagaggaatccaggagtttattttgcagctgattcagtgttgaccaagagtccagctctgggggagtgggaagcaaccaaagcagagacaggtcccagcacaatttttggttttcaagacagcacttctctgtggctttgaaggctatcctagaactgttctttgtatatccttccttgcaactagctcttatagaccaggctggtcttgaactcacagagatccatctgcctctgcctcccaagtgctgggattaaaggcgtgcacctcggctgccaccacccagctacatacataatttacaataataaaaataaaatactttaaagtgttatagcagtttgaatgtaattggccctgtcatctcatagggagtggcactattaggaggtatggctttgttgaaggaaatatgtcactgtgagggtgggctgtgaggtttcctatgctcagggtaccagccagtgtctcagctgaggtcctgttgcctgcaagatgtaggactctcatccctttctccagcaccatgtctgtctgcatgccatcatgttcccagccatgatgacaatgtactaaaacctctgaaactgccacccaactaaatgttttcctttataagagttgccatgctcatggtgtctcttcacagcaatagaaaccctaactaagataagtgtattctcccctactccccatgatttaaaatttaggaaggcaggtaggcaggcaggcaggctggtatagtggttcattctagcacctgagacctggaatgggaggattgtgagttagttctaggccattctggtgcctagaaaccagagccgggggttggcccaatgcagagcacttgctctacgtatggcccagcacaataagtcaatttcctcaccttaaaggcttgacaatttaaaaacactggtttttagttagtccgtgtctgctccacagatggagacagctaatcacagatgcatcaggggccttcctgagtgctaaacatcaaacagccttctcccctcctgagcctttgtgtgcagaatgtgtccatcgcaagaagcaaacagtcttgcttgcccaccaacttccttcctgcatcagaagagctgggtgcaaactgcaagagtagcctcaccttagagatgggtcccattgctctacatgggagcattaccttccaagaaggcaaaaatgtctcctggttgagctttttttgtcacctgttaaaggcaaatcaacagagaggctttgtctcacccactaacatcttggaaacaaataccaacgaacgctggggaggatgtggggaaagcagagccctcatgctctccgagggaaaatcacacccactgtggaacagtgtggaaacctcaaagactgggattacaagcagcacacaagccagccacgctactcctggtcacacaccacaaagacgcttgcacattcacgcttacgctgcgaacactagcaacgttcccactgcctcctttgagccccgccccccgcccctgccccccgccccgcccctgtggtctatgttcctcttccctaaagtcagcttccacttctctgtctccatcttcgccccaccctccctcctcgctacataattgtctctattccatttctctgctttgaaacagctttttgcaaagcatcaaatctattgtcctatgccccaaatcaacctccagtttcacaagtgatacaggaaatcgttttcctaattaaaaatcccccctttgaccatttattcccactcttggaacatcttccccttgaggaaagttacagaatgaggtggctctcctcttcctattcgaggtgtttccttcagactttgtccgtgtctaatctttttaactgttggccaggcctccaccacggcacagatgaactgtggggttcatttacctgaaactctatggaaggatgtttatttctccttcactttagcaaatgataaagggcaccattcactctgtctattctgcaggggccattcctttctctaggccagatactgagaattgctcccagaatcaatgtggtatacatatttccccttcaacattgataggcattgatcacacacacacacacacacacacacacacacacacacagtagcacaaatgtattcccctagcccgcttccatcttgccacaggactccagagtggccctggatagcaagcttcctgttttgtttctctgttcctgctgcttttccaccctccagtctatcttttctaagtccttctgccattgtcctcttcccaactgtcctgagatgcagtcattgtctgggattcagaccttctctctctgcccaagtgagtatattgacccccacggtttgtacaaccataacttcagggagcccgacaaaaactgttttatgagccaagtagtcccaggacttgagaggtagaggcgggaagatcagcagtttgaggccagcctggagagcataagagccggtctcaaaacaacaatggaaactagatactaagtaaaaatcctggggtgtttcatcatgaatgtctgttcttctagtaccacgctgaactccgtacacagctccagctgttacggctttcttagaatccatactctttttttttttttttttttttttttttttttggtttttcgagacagggtttctctgtggctttggaggctgtcctggaactagctcttatagaccaggctggtctcgaactcacagagatccacctgcctctgcctccagagtgctgggattaaaggcgtgcgccaccaacacccggcagaatccatactctttttaaaaaaagatttatcaatttactatgtatacagctttctgcctgcatgtatccatgcatgtcagaagatggcaccaggtcgcattacagatggttgtgagccaccatgtggttgctgggaattgaactcagaatgtctagaagagcaaccagttctcttaacctctgagccatctctccggcccccagaaatccatattcttgaggattttttacaccccccccaccaaaagacgtatatctaaattttaatgtgagaattcacattttcttaagagttgaacatagatttagaggaaaatcagatcccacatgattaacaaagcatgcttgtgggcaggtctgctaccaagaggtgggccgtagcttctagctcagacaaactcactcccttcctcgtggcctcttcgccctcaagtcagaaactcaccctgtgattctgccccagaagttgctctagagcacagtgcatccttccgtcttcactctgtggcttgaattgtgtccatcgcttatgattacaacccctcacagagcatcctaactggtttctttgcatgcctatgggcactcctccattctagaacacccttgccatcaatactatgaaaggaggggtggaggaggaagagcaggaagaggagggggaagcgagggaagaggaagacacggatggcaatgaggaggggggagcacccaagtcctccctggatgagagtctcactgggagacttaatattaattataaatgcttggtcagcagctgggcaggataaggttaggcaggagaaccagactaaggactctgggaagcagaagggcagagtcagacaaggagaggaaacaggaagtacaaggtaaagtcacgtggcagaatgtagataatagaaatgggttcatttaagttggaagagttagctagtaacaagcctgagctatcagccgagcatttataattaatattgagcctccatattggttatctgggaattggcgggcagaaaaaaaaaagtctgcctacaagtcaatgtcatgtagctcccaaagccaaggtacctttgttcagtgcttgactgagccagcattataaattttctccagatgtaccgaatcacatttcatagcaacatgcagacatcaagttttccctgaagctctaaccagctggttgcatgctgtccggagtctcagctataacccagaagtgacctgggtcggggaagaggtggtactttgccttctttgcactctctgtgttgcctcacccattcagcttcaagcaatgtgactgcctgaccctgagggcgtttacaacgcctgacccacagaccacaagtcaaccagctggtgtgctcacgatacctagtctgaaccatagccctgctcccaccctgcctccatctccaccctttcttcactgctcatcacagctggctagcaaagactgcctcagacctgagcacaggctccactccacagccgtgactgttcgagccacttaaatcaaagagcgcttgtcttccgctcagtaaatctctcctcagctcactgatgacgttgactttctctagacagcacatttgggtttaagacactgctacttgagctcttcattcagttcctcagaatacctcatttgggtcagattcccaaagaggaagatagggttcctggcagacagacatgtctcattcctttgaaatccttcagagaaatgcagtgactatggcaccttcttaaaaagcacacacacaaataacacacacacacacacacacacacacacacacacacacacatatccccctcactgtcatccttgatatgtatatgatatatataaaatcattgttttatactgtgataattgattatgaataaaatttactaaaatgaacaattaaaattatggggggggctggagagatggctcatcagttaagagaacagttgctgctcttgcagaacacgagagttcagttcccagcacccacatcaggcagctcataaccatgtgtggtgtcagttccaggagatctggtgccctcttctggcctcctccagcacctgctacatgtggttcacacacacacacacacacacacacacacacacacacacacacacacaaataaatataaagattatttttttcaaaactgagttaaaaataggttctatctgattcatactaaggcttttcacagtggttaagtctattagatatgtctagccatatcctttctcccttctttcttgaggagaggcttttaaagctacaagttacagccttctttgcaaataagagtaccatttaacaggcctctgaccaatgagatgccagaatcggttgcccaggagcttcccaaacagtccattatagggaaaggtggtacaaaccagtagattaggcatgttccacttcctaagtgccgtgccaaataaggaaatggcctcaaatgtttgccttttatcttcacccacctctgaattgcacgctagt

SEQ ID NO: 9 - DNA, 13515 bp, Cricetulus griseus
  source 1..13515: mol_type = genomic DNA
tctagaaacaaaaccaaaaatattaagtcaggcttggcttcaggtgctggggtggagtgctgacaaaaatacacaaattcctggctttctaaggctttttcggggattcaggtattgggtgatggtagaataaaaatctgaaacataggtgatgtatctgccatactgcatgggtgtgtatgtgtgtgtatgtgtgtctgtgtgtgtgcccagacagaaataccatgaaggaaaaaaacacttcaaagacaggagagaagagtgacctgggaaggactccccaatgagatgagaactgagcacatgccagaggaggtgaggactgaaccattcaacacaagtggtgaatagtcctgcagacacagagagggccagaagcactcagaactccagggggtcaggagtggttctctggaggcttctgcccttggaggttcctgaggaggaggcttccatattgaaaatgtagttagtggccgtttccattagtacagtgactagagagagctgagggaccactggactgaggcctagatgctcagtcagatggccatgaaagcctagacaagcacttccgggtggaaaggaaacagcaggtgtgaggggtcaggggcaagttagtgggagaggtcttccagatgaagtagcaggaacggagacgcactggatggccccacttgtcaaccagcaaaagcttggatcttgttctaagaggccagggacatgacaagggtgatctcggtttttaaaaggctttgtgttacctaatcacttctattagtcagatactttgtaacacaaatgagtacttggcctgtattttagaaacttctgggatcctgaaaaaacacaatgacattctggctgcaacacctggagactcccagccaggccctggacccgggtccattcatgcaaatactcagggacagattcttcactaggtactgatgagctgtcttggatgcaaatgtggcctcttcattttactacaagtcaccatgagtcaggaggtgctgtttgcacagtgtgactaagtgatggagtgttgactgcagccattcccggccccagcttgtgagagagatccttttaaattgaaagtaagctcaaagttaccacgaagccacacatgtataaactgtgtgaataatctgtgcacatacacaaaccatgtgaataatctgtgtacatgtataaactgtgtgaataatctgtgtgcagcctttccttacctactaccttccagtgatcaggtttggactgcctgtgtgctactggaccctgaatgtccccaccgctgtcccctgtcttttacgattctgacatttttaataaattcagcggcttcccctctgctctgtgcctagctataccttggtactctgcattttggtttctgtgacatttctctgtgactctgctacattctcagatgacatgtgacacagaaggtgttccctctggagacatgtgatgtccctgtcattagtggaatcagatgcccccaaactgttgtccagtgtttgggaaagtgacacgtgaaggaggatcaggaaaagaggggtggaaatcaagatgtgtctgagtatctcatgtccctgagtggtccaggctgctgacttcactcccccaagtgagggaggccatggtgagtacacacacctcacacatactatatccaacacacacacacacacacacacacacacgcacgcacgcacgcacgcacgcacacatgcacacacacgaactacatttcacaaaccacatacgcatattacaccccaaacgtatcacctatacataccacacatacacacccctccacacatcacacacataccacacccacacacagcacacacatacataggcacacattcacacaccacacatatacatttgtgtatgcatacatgcatacacacacaggcacacagacaccacacacatgcattgtgtacgcacacatgcatacacacacataggcacacattgagcacacacatacatttgtgtacgcacactacatagacatatatgcatttgtatatgcacacatgcatgcacacatacataggcacacatagagcacacacatacatttgtgtatgcacacatgcacacaccaatcacatgggaagactcaggttcttcactaaggttcacatgaacttagcagttcctggttatctcgtgaaacttggaagattgctgtggagaagaggaagcgttggcttgagccctggcagcaattaaccccgcccagaagaagtaggtttaaaaatgagagggtctcaatgtggaacccgcagggcgccagttcagagaagagacctacccaagccaactgagagcaaaggcagagggatgaacctgggatgtagtttgaacctctgtaccagctgggcttcatgctattttgttatatctttattaaatattcttttagttttatgtgcgtgaataccttgcttgcataaatgtatgggcactgtatgtgttcttggtgccggtggaggccaggagagggcatggatcctccggagctggcgtttgagacagttgtgacccacagtgtggggtctgggaactgggtcttagtgttccgcaagtgcagctggggctcttaacctctgagccatccctccagcttcaagaaacttattttcttaggacatgggggaagggatccagggctttaggcttgtttgttcagcaaatactcttttcgtgtattttgaattttattttattttacttttttgggatagaatcacattctgcagctcaggctgggcctgaactcatcaaaatcctcctgtctcagtctaccaggtgataagattactgatgtgagcctggctttgacaagcactttagagtccccagcccttctggacacttgttccaagtataatatatatatatatatatatatatatatatatatatatatatattgtgtgtgtgtgtttgtgtgtgtatgagacacttgctctaagggtatcatatatatccttgatttgcttttaatttattttttaattaaaaatgattagctacatgtcacctgtatgcgtctgtatcatctatatatccttccttccttctctctctttctctcttcttcttctcacccccaagcatctattttcaaatccttgtgccgaggagatgccaagagtctcgttgggggagatggtgagggggcgatacaggggaagagcaggaggaaagggggacagactggtgtgggtctttggagagctcaggagaatagcagcgatcttccctgtccctggtgtcacctcttacagccaacaccattttgtggcctggcagaagagttgtcaagctggtcgcaggtctgccacacaaccccaatctggccccaagaaaaggcacctgtgtgtgactctggggttaaaggcgctgcctggtcgtctccagctggacttgaaactcccgtttaataaagagttctgcaaaataatacccgcagagtcacagtgccaggttcccgtgctttcctgaagcgccaggcacgggttccctaggaaatggggccttgcttgccaagctcccacggcttgccctgcaaacggcctgaatgatctggcactctgcgttgccactgggatgaaatggaaaaaagaaaaagaagaagtgtctctggaagcgggcgcgctcacacaaacccgcaacgattgtgtaaacactctccattgagaatctggagtgcggttgccctctactggggagctgaagacagctagtgggggcggggggaggaccgtgctagcatccttccacggtgctcgctggctgtggtgcatgccgggaaccgaaacgcggaactaaagtcaagtcttgctttggtggaactgacaatcaacgaaatcacttcgattgttttcctctttttactggaattcttggatttgatagatgggggaggatcagagggggaggggaggggcggggagacggagggaggaggggaggaggggaggaggggaggaggggaggaggggaagggatggaggaaaatactaacttttctaattcaacatgacaaagattcggagaaagtgcaccgctagtgaccgggaggaggaatgccctattgggcattatattccctgtcgtctaatggaatcaaactcttggttccagcaccaaggattctgagcctatcctattcaagacagtaactacagcccacacggaagaggctatacaactgaagaaataaaattttcactttatttcatttctgtgactgcatgttcacatgtagagagccacctgtgtctaggggctgatgtgctgggcagtagagttctgagcccgttaactggaacaacccagaactcccaccacagttagagcttgctgagagagggaggcccttggtgagatttctttgtgtatttatttagagacagggtctcatactgtagtccaagctagcctccagctcacagaaattctcctgttccggtttccaaagtactggagttatgagtgtgtgttaattgaacgctaagaatttgctgattgaagaaaacctcaagtgggtttggctaatccccacgaccccagaggctgaggcaggaggaatgagagaattcaaggtttgccagagccacagggtgagctcaatgtggagactgtgagggtgagctcaatgtggagactgtgagggtgagctcaatgtggagactgtgagggtgagctcaatgtggagactgtgagggtgagctcaatgtggagactgtgagggtgagctcaatgtggagacctgtatcaagataataatagtagtagtaacaatgcaggcgagggtgtggttgagtggtagagcagttagttgatttgacatgcttgaggtctcccggtccatctgtggccctgcaacaggaagggagggaggaagggggggaacgagagagaggaaagagagacagaagctaagatagggaatgagagaggaaggaagaaacgggaagaaattcagactccttcctgagttccgccaacgcctagtgacatcctgtgcacaccctaaggtggcctttgtgtggcactggcttgggtggtcgggaaaggcattttcagcttgttgcagaactgccacagtagcatgctgggtccgtgaaagtttctgcccgttaacaagaagtctctactacttgtgacctcaccagtgaaaatttctttaattgtctcctggtgttctgggttttgcatttttgtttctaaggatacattcctgggtgatgtcatgaagtccccaaagacacagtggggctgtgttggattgggaaagatgatttatctggggtgtcaaaaggaaaagaagggaaacaggcacttgggaaaatgtcctcccgcccacccgaattttggcttggcaaccgtggtggaggagcaagaaacacgtggacgtttgaggaggcatggggtcctaggaggacaggaagcagaaggagagagctgggctgacagcctgcaggcattgcacagtttcagaaggagattacagcatgactgagtttttagggatccaacagggacctgggtagagattctgtgggctctgaggcaacttgacctcagccagatggtatttgaataacctgctcttagagggaaaacagacatagcaaacagagccacgtttagtgatgaaactctcactttgcctgagtcatgtgcggccatgcccaggggtcaggctgacactcaactcaaaaacaagtgagaaattgaagacaatccgtggtggcagctactggaagggccaccacatccccagaaagagtggagctgctaaaaagccatttgtgataggcacagttatcttgaatgcatggagcagagattacggaaaaatcgagaatgttaatgaggcaacattcgagttgagtcattcagtgtgggaaacccagacgcttccatcccctaaaaggaacatcttgctctcagtcaaaatggaaataaaaattggggcttgaatttggcaaatgattcagaactctgtgtaggtattttcacacgcacagtggataattttcatgttggagtttatttgtgctaaaaggcagaaaagggtaaaaagcacatcttaagagttatgaggttctacgaataaaaataatgttacttacagctattccttaattagtacccccttccacctgtggtaatttcctgagatagtcagtggggaaaagatctctccttctcttctttctccccctcccctcctctccctccctccctccctccctccctcctctccctccctccccctttccttctttctttgctccttctcctctgcctccttctccctttcttcttcatttattctaagtagcttttaacagcacaccaattacctgtgtataacgggaaaacacaggctcaagcagcttagagaagattgatctgtgttcactagcgtgcaattcagaggtgggtgaagataaaaggcaaacatttgaggccatttccttatttggcacggcacttaggaagtggaacatgcctaatctactggtttgtaccacctttccctataatggactgtttgggaagctcctgggcaaccgattctggcatctcattggtcagaggcctgttaaatggtactcttatttgcaaagaaggctgtaacttgtagctttaaaagcctctcctcaagaaagaagggagaaaggatatggctagacatatctaatagacttaaccactgtgaaaagccttagtatgaatcagatagaacctatttttaactcagttttgaaaaaaataatctttatatttatttgtgtgtgtgtgtgtgtgtgtgtgtgtgtgtgtgtgtgtgtgtgtgtgaaccacatgtagcaggtgctggaggaggccagaagagggcaccagatctcctggaactgacaccacacatggttatgagctgcctgatgtgggtgctgggaactgaactctcgtgttctgcaagagcagcaactgttctcttaactgatgagccatctctccagccccccccataattttaattgttcattttagtaaattttattcataatcaattatcacagtataaaacaatgattttatatatatcatatacatatcaaggatgacagtgagggggatatgtgtgtgtgtgtgtgtgtgtgtgtgtgtgtgtgtgtgtgttatttgtgtgtgtgctttttaagaaggtgccatagtcactgcatttctctgaaggatttcaaaggaatgagacatgtctgtctgccaggaaccctatcttcctctttgggaatctgacccaaatgaggtattctgaggaactgaatgaagagctcaagtagcagtgtcttaaacccaaatgtgctgtctagagaaagtcaacgtcatcagtgagctgaggagagatttactgagcggaagacaagcgctctttgatttaagtggctcgaacagtcacggctgtggagtggagcctgtgctcaggtctgaggcagtctttgctagccagctgtgatgagcagtgaagaaagggtggagatggaggcagggtgggagcagggctatggttcagactaggtatcgtgagcacaccagctggttgacttgtggtctgtgggtcaggcgttgtaaacgccctcagggtcaggcagtcacattgcttgaagctgaatgggtgaggcaacacagagagtgcaaagaaggcaaagtaccacctcttccccgacccaggtcacttctgggttatagctgagactccggacagcatgcaaccagctggttagagcttcagggaaaacttgatgtctgcatgttgctatgaaatgtgattcggtacatctggagaaaatttataatgctggctcagtcaagcactgaacaaaggtaccttggctttgggagctacatgacattgacttgtaggcagacttttttttttctgcccgccaattcccagataaccaatatggaggctcaatattaattataaatgctcggctgatagctcaggcttgttactagctaactcttccaacttaaatgaacccatttctattatctacattctgccacgtgactttaccttgtacttcctgtttcctctccttgtctgactctgcccttctgcttcccagagtccttagtctggttctcctgcctaaccttatcctgcccagctgctgaccaagcatttataattaatattaagtctcccagtgagactctcatccagggaggacttgggtgctcccccctcctcattgccatccgtgtcttcctcttccctcgcttccccctcctcttcctgctcttcctcctccacccctcctttcatagtattgatggcaagggtgttctagaatggaggagtgcccataggcatgcaaagaaaccagttaggatgctctgtgaggggttgtaatcataagcgatggacacaattcaagccacagagtgaagacggaaggatgcactgtgctctagagcaacttctggggcagaatcacagggtgagtttctgacttgagggcgaagaggccacgaggaagggagtgagtttgtctgagctagaagctacggcccacctcttggtagcagacctgcccacaagcatgctttgttaatcatgtgggatctgattttcctctaaatctatgttcaactcttaagaaaatgtgaattctcacattaaaatttagatatacgtcttttggtggggggggtgtaaaaaatcctcaagaatatggatttctgggggccggagagatggctcagaggttaagagaactggttgctcttctagacattctgagttcaattcccagcaaccacatggtggctcacaaccatctgtaatgcgacctggtgccatcttctgacatgcatggatacatgcaggcagaaagctgtatacatagtaaattgataaatctttttttaaaaagagtatggattctgccgggtgttggtggcgcacgcctttaatcccagcactctggaggcagaggcaggtggatctctgtgagttcgagaccagcctggtctataagagctagttccaggacagcctccaaagccacagagaaaccctgtctcgaaaaaccaaaaaaaaaaaaaaaaaaaaaaaaaaaaaaagagtatggattctaagaaagccgtaacagctggagctgtgtacggagttcagcgtggtactagaagaacagacattcatgatgaaacaccccaggatttttacttagtatctagtttccattgttgttttgagaccggctcttatgctctccaggctggcctcaaactgctgatcttcccgcctctacctctcaagtcctgggactacttggctcataaaacagtttttgtcgggctccctgaagttatggttgtacaaaccgtgggggtcaatatactcacttgggcagagagagaaggtctgaatcccagacaatgactgcatctcaggacagttgggaagaggacaatggcagaaggacttagaaaagatagactggagggtggaaaagcagcaggaacagagaaacaaaacaggaagcttgctatccagggccactctggagtcctgtggcaagatggaagcgggctaggggaatacatttgtgctactgtgtgtgtgtgtgtgtgtgtgtgtgtgtgtgtgtgtgatcaatgcctatcaatgttgaaggggaaatatgtataccacattgattctgggagcaattctcagtatctggcctagagaaaggaatggcccctgcagaatagacagagtgaatggtgccctttatcatttgctaaagtgaaggagaaataaacatccttccatagagtttcaggtaaatgaaccccacagttcatctgtgccgtggtggaggcctggccaacagttaaaaagattagacacggacaaagtctgaaggaaacacctcgaataggaagaggagagccacctcattctgtaactttcctcaaggggaagatgttccaagagtgggaataaatggtcaaaggggggatttttaattaggaaaacgatttcctgtatcacttgtgaaactggaggttgatttggggcataggacaatagatttgatgctttgcaaaaagctgtttcaaagcagagaaatggaatagagacaattatgtagcgaggagggagggtggggcgaagatggagacagagaagtggaagctgactttagggaagaggaacatagaccacaggggcggggcggggggcaggggcggggggcggggctcaaaggaggcagtgggaacgttgctagtgttcgcagcgtaagcgtgaatgtgcaagcgtctttgtggtgtgtgaccaggagtagcgtggctggcttgtgtgctgcttgtaatcccagtctttgaggtttccacactgttccacagtgggtgtgattttccctcggagagcatgagggctctgctttccccacatcctccccagcgttcgttggtatttgtttccaagatgttagtgggtgagacaaagcctctctgttgatttgcctttaacaggtgacaaaaaaagctcaaccaggagacatttttgccttcttggaaggtaatgctcccatgtagagcaatgggacccatctctaaggtgaggctactcttgcagtttgcacccagctcttctgatgcaggaaggaagttggtgggcaagcaagactgtttgcttcttgcgatggacacattctgcacacaaaggctcaggaggggagaaggctgtttgatgtttagcactcaggaaggcccctgatgcatctgtgattagctgtctccatctgtggagcagacacggactaactaaaaaccagtgtttttaaattgtcaagcctttaaggtgaggaaattgacttattgtgctgggccatacgtagagcaagtgctctgcattgggccaacccccggctctggtttctaggcaccagaatggcctagaactaactcacaatcctcccattccaggtctcaggtgctagaatgaaccactataccagcctgcctgcctgcctacctgccttcctaaattttaaatcatggggagtaggggagaatacacttatcttagttagggtttctattgctgtgaagagacaccatgagcatggcaactcttataaaggaaaacatttagttgggtggcagtttcagaggttttagtacattgtcatcatggctgggaacatgatggcatgcagacagacatggtgctggagaaagggatgagagtcctacatcttgcaggcaacaggacctcagctgagacactggctggtaccctgagcataggaaacctcacagcccaccctcacagtgacatatttccttcaacaaagccatacctcctaatagtgccactccctatgagatgacagggccaattacattcaaactgctataacactttaaagtattttatttttattattgtaaattatgtatgtagctgggtggtggcagccgaggtgcacgcctttaatcccagcacttgggaggcagaggcagatggatctctgtgagttcaagaccagcctggtctataagagctagttgcaaggaaggatatacaaagaacagttctaggatagccttcaaagccacagagaagtgctgtcttgaaaaccaaaaattgtgctgggacctgtctctgctttggttgcttcccactcccccagagctggactcttggtcaacactgaatcagctgcaaaataaactcctggattcctctcttgtaacaggagcccgaagtcaggcgcccacttgtcttctcgcaggattgccatagactttttctgtgtgcccaccattccagactgaagtagagatggcagtggcagagactgggaaggctgcaacgaaaacaggaagttattgcaccctgggaatagtctggaaatgaagcttcaaaacttgcttcatgttcagttgtacacagactcactcccaggttgactcacacgtgtaaatattcctgactatgtctgcactgcttttatctgatgcttccttcccaaaatgccaagtgtacaaggtgagggaatcacccttggattcagagcccagggtcgtcctccttaacctggacttgtctttctccggcagcctctgacacccctccccccattttctctatcagaaggtctgagcagagttggggcacgctcatgtcctgatacactccttgtcttcctgaagatctaacttctgacccagaaagatggctaaggtggtgaagtgtttgacatgaagacttggtcttaagaactggagcaggggaaaaaagtcggatgtggcagcatgtacccgaaatcccagaactggggaggtagagacggatgagtgcccggggctagctggctgctcagccagcctagctgaattgccaaattccaactcctattgaaaaacctttaccaaacaaacaaacaaacaaataataacaacaacaacaacaacaaactaccccatacaaggtgggcggctcttggctcttgaggaatgactcacccaaacccaaagcttgccacagctgttctctggcctaaatggggtgggggtggggcagagacagagacagagagagacatgacttcctgggctgggctgtgtgctctaggccaccaggaactttcctgtcttgctctctgtctggcacagccagagcaccagcacccagcaggtgcacacacctccctccgtgcttcttgagcaaacacaggtgccttggtctgtctattgaaccggagtaagttcttgcagatgtatgcatggaaacaacattgtcctggttttatttctactgttgtgataaaaaccggggaactccaggaagcagctgaggcagaggcaaatgcaaggaatgctgcctcctagcttgctccccatggcttgccgggcctgctttctgcaagcccttctctccccattggcatgcctgacatgaacagcgtttgaaatgctctcaaatgtcactttcaaagaaggcttctctgatcttgctaactaaatcagaccatgtttcaccgtgcattatctttctgctgtctgtctgtctgtctgtctgtctatctgtctatcatctatcaatcatctatctatctatcttctatttatctacctatcattcaatcatctatcttctaactagttatcatttatttatttgtttacttactttttttatttgagacagtatttctctgagtgacagccttggctgtcctggaacccattctgtaaccaggctgtcctcaaactcacagagatccaactgcctctgcctctctggtgctggggttaaagacgtgcaccaccaacgccccgctctatcatctatttatgtacttattattcagtcattatctatcctctaactatccatcatctgtctatccatcatctatctatctatctatctatctatctatctatctatcatccatctataatcaattg

SEQ ID NO: 10 - DNA, 14553 bp, Mus musculus
  source 1..14553: mol_type = genomic DNA
cttgaagaacacatgttttccaagagggagcacccatgttggaatgacaatgtagttagtgctcctctcctgtaggttagtgctcctttgctataggtaagtgctcctctcctataggtcagtgctcctctcctataggttagtgctcctctcctataggttagtgctcctctcctacaggttagtgctcctctgctctaggttagtcctgctctcctatagtacctagagagctagggcaaatgggctaggcccgaagtgcagagacaaacagctatggaagactgggtaagcacttccaagctacgaaagagcagtgtgaagggtcagggcttgtgcagttagtaggggagatcttccagttgaagaaacagaagaactgagagccactgggtatcatcctcctgcgccatgccttcctggatactgccatgctcccaccttgatgataatggaatgaacctctgaacctgtaagccagccccaatgaaatattgtttttatgagagttgccttggtcatgctgtctgttcacagcagtaaaaccctaaataaggcagaagttggtaccagtattgctgtgatagacctgaccatgctttcctttgaaagaatgtggatttggtgactttggatttgcaacacagtggaatgctttaaatggagattaatgggtcatcaattcctagtaggaatatggaagactttgttgctgggagtatttgaactgtgttgacctggcctaagagatttcaaaggagaagaatttcagaatgtggcataaagacagtttttgtggtattttggtgaagaatgtggctactttttgcccttgtctgaaaagtctgcctgagactaaagtgaagagaatcagattaattgcattgacaagggaagtttgtggctgcgctatctggaaacttacagccagcctcttggacctcgggtgacttacgcaaatactcagggacagagatgcttgactctgtactgatgagttgtcttggatgcaaatatgggctcttcatttgactacatgtcacgatgagtcaggagctgctctctccagagtgtgacaaagcgaggggatgctgacggtagctgttctagctttgaaggtaagcctgcacttatgctaaagtcacacatacacgagccgggtggagaacctgtctgtgtggagacacctttcattacctgtggcatccagcctctcaagcttggactgcctgtgtgctcctggactctggaggtcccactgctctgtcctctgctgcttatgatactgacattttaaaagaatccagtggttcccccctgtactcggtgtctacttctacctggatgttcctcatttatgttctgtgacacttctctgtgactctgctgcattcctgggtgacatgtggacaccctgtccctttgcagaccatgatgtcactgtcactagtggaatcagatgccccaagtgttgtcctgtgtttgggaacgtgacaggcagtacagaagcagaagaggaagggtgaaaacggaaatgtcacagcagcatctgatgtgtgcctcagtcacgcatgctgctgattggaactactcagcatgagagagggccatggtgaatacacaaccctatacacactgtgtccatttctctctctctcttacacagagagagagggaggagggggagggggaggcggagggggagggggagggagagggagtgggagagggagagggagagggagagggagagggagagggagagggagagggagagtttaatgtctgtgaagagataccatgaccaaagcaactcttataaaggacaacatttaattggggctggcttacaggttcagaaattcagtccattctcaccatggtgggaagcatgcaggtagatgtggtgctggaggaaccaagagttctatatcctgatctgaaggcagccaggagaagactgcctcttctgcacagggcagagcttgagcatagaacatcaaagcccttccccacacttcctccaacaaggtcatacatacttcaacaaagacacacctcctaacggtgccactccctgtggaccaaccatttaaacgcatgagtctatgagggtcaaagctcttcaaaccaccacactcatgtacacacacacacacacacacacacactctcatacacacacacacacacactcacacacacacacacacacacacacacacacacaccacacacacacacacacacagagttctattttgcactgtttcactgtcacaaggttctacttatctcagacacactgccaggaattgtgtgggaagactttcagtttctttgggttcacatggacttagcagttcttggtgatcctgaaagatttctgcagaaagaagccaaagtgttgagcccaaggcctggccacacattagtcctgtctagatgaacaggggtttaaaaataagggggcatcaaggtgaagccagcaggggctgacttagagaggagacccacccaagccaactgctcgaagtcaaaagcgatgaatccccatatccagctgtgcccggtgctgtcttgctacatctttagtaaatgttcttttagttgtatgcgtatgaatattttgcttgcatatatttgtgtacaccataggtgttcctagggcctatggaggccagaagagggcatcagatcctttggaactggaattatagacacttgttacccatagagtagattgtgggaaatgagcctttagtcttcgagagcggccagtgctcttaacctttggtcgtttctccaggtctttgagactttattttcttggacatcaggacaggatccagggctttgagcttgtttcttcagccagctttcttttcatgtatattaaattttatgttattttgctttctttttccccaagacagaatcacactctatatagctcaggctgggtttgaattcagtttccctgtctcagtctaccgggtaatatgattacagatgtgagtctgactttggtatcaaagtccccagcccttctggatatgtgttttaaggatatcagatatatccttgatttgctttgaattttctttttagttacaacataattagttccgtgtcacctgaatatgtgtatgtcacctacatagtcttccttcttctcttcttccctctcccaccttcccaggtacctgtctgtcttcatatccttgtgctgagagtcttgttgagggagatgatgaccgagacagagccactggggaagggagatgggctagtgcaggtcttcagagaggagctcgtgaatattgtagcccctttagtccctggcatgtcctcttgtatagccaccgccatgctgtggcctggcagaagtgaataagttgtccagctgttgacaggcctgccctccagacccagtctgatcccaagaaagggcatctgtgtctgtctctgaggccgtaagtgctgcctggttgtctccagcttgacttgacactccctccttaataagagtaccacagaacagggtctgcagagtccctgggccaggtccctgtgctgtcctggaatgccaggcgtgaatttcctgtgaagtaggactttgctcgccaagctcccacggcttgcccttcagatagccagaattatctggtaccctgcattgccgttcaatacgcagagtatcactggaagcgcgcgcgcgcacacacacacacacacacacacacacacacacacacacacacgcccactccatctttaaaccccaccccccagcaacggcggtgtaaacactctccatcaggaagctgaaacgcagttgccctctgctggggagatgaaggcagcttgctgggggcgaggaccgtgctagcaaccttccctggtgcacacgggctctggtgcatgacgggaacggaaacgcggaactaaagtcagtcctgctttttttttttttttttttttttttttttttttttttttttttttttggcgttggtggtggactgagtgacaatcagtgaaatcacttaggttgtttttctcttcttcgttgggtttgatagacggtgggagagggtcagaggagaaggggagggatggggagagagggaggagggaggggcgggaggcggggggcgaggaaaacgtgctaacttctccaatcctacaagacaaaggtttggagaaagccgcactgagtgacccagcagaaggaatccaggaatgtccgctggaatctgactgttgattccagcgccatgcagagaatctaggctggtaggaacattctttgtcctatccgacataataactccaaccaacacggaaaagaaaggctatacaagtgaagaaatggcattttcactttcatgactatacaatcacttccaggtagtaacacgtgtctagcacagcggttctcaacctgggggtcacgatcccccacttttctgcatatcagacatttttacgttgttattcataacagtagcaaaattgcagctatgaagtaacaatgaaatgcatttatggtgcgtgtgtgtgtgtgtgggggggtatcaccttaacatttactgtaagaaggttgagaatactgctccagcagctagtgtgttggacttaggttctgggtatattattagcaatagccaaccagaatccccacccaccacagcattgaggccccatgcagggcttgctgggagaggcactgataagacttctttatgtatttatttagagacgaatactcattaggtaggccaagctagcgtcaaactcatggcaattctcctcctccagtttcctaagtactggactcaggagtgtgttgccatcatatacagtaaggatttattgactgaagaaaatctcaagtggctttggttaatccctactacgccagaggctgaggcaggaggcgcgcaaggtcaaggcttgcctgggctacatatagagtgagctcaattttgacacttggtgcggtgttagtagtaatagtaaagatgaaggtgtggctcaggtggggccggtgattggacacacttggggtctcctggtccatctgcagctgtgcaacaggaagagcggagaatgagaggaaagagagaaaagacagaatgagagagagggaggaagagagaaaaaggaaaagagagaggaaaggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaaaggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaagggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaagggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaagggaaaatggaaaatgaggaaagcgagaaagaagaaatgagaaagaggaaagggagaaagaaatgagcgagataaaagacagaatttgagagagggaggaagaaataggaaaagagaggaaaggatggagaaaagagagaaagaaagagagatgaaagagagaaaggagaaatgaaatgagagagagagagagacacaaagagccagagagagaagaaaaaaggggaaagagaaagagaaagaggaaggctcctcttggacacatcttcctttatctttccctggggaccgccaaagcctggtggcatactgtacattctgtacactgttcattcaaaacaggctctgtcttaaagatggtctgagcggtcagaaaagggtattgttaacttgtttgcaaaactgcctcaggagagtgctgagtgcgtgaaagttgctgcccgttaaggagaagtctctactacttgtgatctcaccatcgaaaatttctttaattgtctcctggtgttctgggttttgcagttttgtttctaaggatacattcttgggtgatgtcacaaagtccccaaagacacggtggagctgtgttagatggggaaagacagtctgctgaggatttatctggaactgtcagaaggaaaagaaggtaaatggggcacttgggaaagtggcctctagtttgacttctggcttagcaaaggttgtggggagataaggcatacacagtagttagcaggaggcaacagggtcctgggaggacgcgaggcagaaggagaggctgggctgacagcatgcaatcattgcatagtctccaaaggagattgcaacatggctgagttttcagaggtcctacagagcccgtggtagagattctgtgggttctgagacaacttgactttagccagatggtatttgagtaatctgggagagagaaaacagctacagcaaacagggccacatttagtgacgaaactctcactttgactgttgagtcatttgcagtgggccctgaggtcaggctggccctcagctcaaaaacaagcgaggaactgaagcaattactcagataatccacagccacagccactggaaagggccacatccccagagacagcacagcaggggtgggggtggggctatgagaaagttagtgattgtagcagttatctagaatgtgcggagcagaggaggttacacaaaaacctagaatgtcattcaatgtgggaaaccgagaggctcccaagccctaaaaggaacagtttgctttcagccaaaatggaaataaaatttggggcttaaatctggcaaatgattcagaccttctgtgtaggtgtctttaaatgcacagcagattgattttcatgttggagtttatttgaactaaaagacagaaatggtgaaaagcacacctgaagaaattgagatgctatgaataaaatcatttacttacagctatcacttaattagtacctccttccaccttgctgatttattgggctagtcaaggaagaaaagatcttccctcctccttctctcctcctccccctcctctcctcctcccctcccctccttgaccttcctctcctccttttccctcctccccctcttcttctcttcaccccctcctcccctcccctcctctgtactcctcccctttcctcccaatctcttttttctcccccttcttctctttctcccccctcctcttccctcctcttcctccctccctccctcctcctcctcatcctcctcttcctcttcatcctcttctccttcctccctctcctcctcctccttttccagccctacctaccttccctttcttcttcatttattcaaagtagctttgaacagcactactcggtttagttgtgtataaaaggaaaatgcaggtccaagcagcttggggaagattgctttttgctctctggaggcagatgatgacagttcaagatcattccttttgctccatgtcacaggaagggggacatgccgaatctaccagtttgcagccacctacacaggatccaccttcacttctaaggaaatgtttgggaagctacctaccaaccacttctggcatctcatgggctagaggactcttaaatggcactcttatttgtttaataaaggaggttgtgacgtgtagttttaaatcccttccacacaacaattgctactctctgaccaaaaaagaagggagacaggatacggctaggtgtctagtagactttaccactttgaaaagccttaatataaatcaggtagatacatctttttaacttattcttgtaaagacaaaaacaaaactttatttttatttgtgtgtatgcttgtgtgtgtgtgcctgtgtgtataccacatgtcgctggtgccggagaacaccagaagaggggacctgatctcctggagctaaagctatccatggttctgagctgcctgatgtgggtgctgggaacagaactctggtcttctgcaagagcaacaagcctcctcttaactacgaatctcctccccatccccccaaatacatttaattattcattttagcagctttatttcgtaactacttatcacagcataaaacaaggattttatatatattacatgcaatcgaggataagagttgaggggagatgcgtgtgctccttctgggtgtctgtgcttttgaagaatgtaagcagtgcacaagggaccgaggcgtgcctgtctgccaggagctgtcttcttcccttggactctgagctgagtgcagtgctccgaagaagtaaaagacgacctcatgaagcaatgtcttcaacccaaacatgctgtccagacaaagtccagcttcattagtgctctgaggagagacttactgagcctcaggaaagcccccctcagcatggcgaaagtccactttgattgaagtgactcgaaagccatggcagtgcggcggcggccgcgtggagcttgtgctcgagtcggaagcggcatctttgtcaggcggctgtgattagcacggggaggcaggactggagtgaaggaagagttgggggcggggcttagcgctctggtctcctaagctgtagtcagcgcctcaagatttgtaacctgccttctgccttcccagccaggcagtcaagtggctccaagctgaagactgcaaagtgcccctaaccttttggttatagcgaggctgaagacaccgtgctctttcatgaaagccggatgtctgaaatccgatttgataaatatggataaaacgtataacgctcgatcaatcgaatcgaaggagctcacgattggcaccacggctttggggacaacagagtactgactcgttgggaggacttggatacttcccctcctcttccatctcttcccctttcctcacttcctcctccttccttctccattttctccctcttcactgtttcttactatttttacaaaagattttatttatttatttatttatttatttatttatttatttatttatttatttatttaatgtatgcgagtacactgtagctgtcttcagacacaccagaagagggcgtcaagttccattagagatggtttcgagccaccatgtggttgctggggcctctggaaggaccgccagtgctcttaacccctgagccatttctccagtacccttctcaccgtttctcttcaatcttcttcctcttccttctccactttccttgtcttcttggtttcattatctttctccctttcttcctcttctccccttcttcctcctccactgtagttttccttccctactcttttcctgcctccctcctcctcccctctcattccccctcctctttcctccttctccctcctcctccttccttctccctctcccctctcccctctcccttctcccttctccccctcctcttcctctttctccttctccacccctcctgtcacagtatcaatggcaagggtgttctagaatggaggagtgtcccctaggcactaacgaaagccagttaggatgctctgagacgggtacaattcagggagggccgtggggatggaagggttgtgctgcgattcattctggagcaacccccaggcagaatcatgaggttggttccggattcgcagggcacaattcagaagaggaaggtttcaggaaggacgagtttgtctgagataggagttacatctgatgtcttggcagcagagccactgtacaagcgtgctttattaaccacgtgggattaaatcttcttttaaatttattttcaactcttaaggaaacgtgaactttcacattcaaatttagacttgcagctcttatggggaaaaaaaggggatcttaagaatattaagcataggcggctggagagatggctcagcggttaagagcactctctgctctcccagaggtcctgagttcaattcctagcaaccacataatagttaacaacagtctttaatgaattctaatgccctcttctggtgtgtctgaagacagttacagtgtactcatataaataaaataaagaaatttaaaaaaatgaatattaggcatagattcctggatcctaagaaagccatcagagctggagccatgtgtgggatcctgcttggtgctggaggggcagagttcatgcccccggggtttttacttattatcacattttcatcgttgttttgaaacagggtcttgtgtggtccaggctggccttgaactcatctttcagcctctacctcacaggttctgggattacttggttcctaaaagtatctccgtcaagctccctggtgttatggctgtgccaaccaggagggtctatacactcgctcaggtagagggagaagatccgaatctctgacagggactgctgcctctcggggcaaatggagtgaaggacagcggcagaaggatttaggaaagatggacgggagagtggaaatgctgcagaagccagaaaacaaagcaggaagcctgctgtccagtggggctcaagagcggagggatgcgagggggctgcgcaggaacatttagcgtctgcgtctatgggggtaggggcggggtgccagcacctagtcacctgaaggggaaatgcttgcccagggagcaggtctcagtagctgacctagagaaaggagcggcccctacagaggagacacgggtcactgtttgttaaagtgaaggagaaataaatattctttcaaagaatcttaggtgagcccagttcatctgcgctgtggaggcctggggaacagttaaaaagaccctgacacacacccaaggcaaacaagcaacacacggctccttccgtaagggtccatgattctctgaagaatcagccccggaatcagccccggaatcaggtagtccgtaaacacaatgagtgttttactctgcagaagtccagcctgctggcgtctcccattaccaaaatagagggatagtcacgtgagctcaccggctcgatttaaggcacgtggttttccagggtagatgagctttggcttctggaaccattatggggcacgaaggatggagccaggattttttttttttttttttttttctattagcaattgatttgcttgggcttggctggacttgcccagttcttaggcccagtcttcttaactgccgatctgaagtctgtcatggagtcagcctagccttctcacttcccttcagctcgaataggaagaggaggtgcacaccagatggtctgagagcagggataaatggtgtgcctttgtctttcagtatttcgttattttaagtaggaagatgcttttctgtattacattgcttgtgaaaccggaagttgattcggggcacaggacaatggatttggtgttttgcaaggactgtttcagaagagagaggagtggaagggtggttagagtgaggagtggggtgggacgggatgggggaagagaaggaagggccagacaggctaggtagggctgagaggaggcggtgggaacttcttgagttagcgcagcagtaaacttggatgtgcgtgtatctttgtgatatatgacccggagccgtgtagctggctccgatagtactgctaatgtcagtgtcggggggggggggtcccatactgttccacaggggctgcacattcccatcgagagcaggagggctcctctctccatacatcctcgccagcattccttgttgtttctgtgatgacagggggtgggatgaaatctctctgttggtttgagagaccgtgaagaagctcaaccccaggacattttgcagtcttggaaggcagtgcctccatgtggagccgtggagcccatctctgagtccaggtcactcttgcagttcgcactcagctcttcagatgcaggagagacgttggtgggaaagcaagattgtttgcttgttgagatagacacattctccacacaaaggctcacgtggggcaaaggctgattgacgtacagcgttcaggaacgcctgtggtagagctatgattagctgtctccatctatgaagcagacaaagagttataaaaaaaatcaatgttttcaaattgtcaaacttttaacccgacagcaagcgctctgtccctgggctaatccctagccctggtttcttgagatggggtcttttgtgcactagactggcctagaactcacgatcttagtgttccagcctcccagctgctgggatgagccgctataaccagtctgcctgccttcctaaattttaagtgatgggaagtgggggagaatacagtttaaagtatgcagatctgagagcaggaacctggcaaagccaaggggccggagttacaggcggctaacatgggtgctgggaactgacccaggtccttgagaggagcagtgtgtactcttgaccaaacaggtccgtctctccagtccccgtagtattaaaaataggtactacgggcatggtggtgcacacctttaatcccagcactagggaggcagaggcaggtggatttctgagtttgaggccagcctggtctacaaaatgagttccaggacagccacggctatacagagaaaccctgtcttgaaaacaaaacaacaacaaaataggtactacaaagcgatgtaattgtgctcaaacatgcaaaccgaggggactgtatgcataagaaagagaaagacggccacactggttctatctgggtgacaggaaatcagtatttttatttttcacattcatttttttgttgttgttgttgacacagtgatttttctatcaaaaacattatttcttttatagttcccctgaggagctgtttttaaagccgtgctttgaaaaaccattgaaggagcagaggcagggagactcctgtgtggcagtcggtgaagcaggccctctgcaggcaggctggccctggacttgggagtctctttccctccctcctgtgctcaaatagcaaatgtcaggcttcaatgtagctagaaggttctagaatgattaagtttccaaggctgaagagcttccctgtttgcctttcacttccctggagaggtcgttgtgtgttccggagtctgcaaggtgcctttggtgatgcgggtggttcatctcgggagattccgcctggaggacccaagttcaagccctgcctgagctacagagtgactttcaggtcttctgcgcaattcagtgagacccagtctacaaataaaaagtaaaaagaaggctgtggatggaactcggtggtagagttctgggtttactccctagaggaggggagaaggaggaggagggaggaggaagaggaagaaagaagaagagaagggaagaggagaaggaagggagggaaggggctgacaagaagagagaagagggagggaggggagggaaaggaaggggaaaggaagggagggaaggggctgacaagaagagagaagagggagggaggggagggaaaggaaggggaaagaagagaagggtaagaagaaactgttccaatggtctgggccacagagtgatggccttttgtggtgatcagctgtaatccttgatttgacacaacctagaatctgggaagcgagtttctgtgaaggagcattcacactggctggcctgtgggcgtgcatgtgggagactgtcataattaggttcattaatacaggaagtcccagcccactacaaatggcttcgttccatacccaagagatgctaactgtagacggttggagaaagcaagcaagctgtggataccccacgctctttcacctcggctcctggggggtgggtgcactgtgtctcttggtattttaaagtcctgccttgacgtccctgctgtgacagactgtaactggaattgtgagctttagtcctttagttttctacgttggtttttctcaggatattttatcgcagtaacagaaacaagaccaggacacttgatctcctctgatcaacactgaagagttacaaaacaggctgaggaaacaaactttcttctccctctcccccttctgtccctccccttccttctcgctccctcccttgccccctctctccctgtctctgtctctgtctctgtctctgtctctgtctctgtctctgcctctcccctcccctcccctccctctgtctctgtctctgtctctgtctctgtctctgtctctgtctctgtccctttctcctctatctcctaaatggctggaggccatgctagctcaatgttgaactttgaacacgtatttaggaaatctttgttcttaacagttctgaagtgctgaagtggtggtttagtctctcggcctgacaagctcacttcctctcactctgtcttaatgaccaaatctgccatttccctaaaacagcacaggctccagctccaggttgctccggagcggag

SEQ ID NO: 11 - DNA, 4001 bp, Cricetulus griseus
  source 1..4001: mol_type = genomic DNA
ccaagatgcccatcaactgattaatagatgataaaattattgtacatttcagtgtaatattattcagtttttaagaaaaatgaaattatgtaataagcatgtaaatggatatatcttgaaacaaccattccccattatattacctaaacattgaaagtccaaaatcatatgatctttttagtggatctactaatcttttgctatatgtattttattgaactacccatggatgtgagataattggtaacaacagcacatgggagagcatgggatcattcaaggaagattagagagaatgcattttttaggagataatggaggagcaatagaaaggattaaatgaggttactgatgaaagtgatggttagagaaggcaatatgaggagggataactagcacttagggccttttgaaaaagacatagagaaaatactattgtagaaacttcctataattggtgtatagttatatacaccaaagagctcagatggagttaccctataatggaaatattaactactttttatcactgtgataaaacatcctgaacagagcaacatagattgggaagcatttactttggcttacagttctaacgggataaaaattcatgatgaaagaatgaatatgtcagcaaacagcagtagcaatggcctgagaagcaggtgagagctcacatcttgaagtgtaagaatgtagcagagagaacaaactgcaaatgaccagaaaatgcttttggatcagagcccatacccctctgactgacttctccagaaattctgaacaaataaaactccccaaacagagccataactgaaggtccagtgtctgagactactaggggtatttcttattcaaaccactacaatggggtggggggagcaatcctccaagtaggcactacacacagacaaataaaaactctagtaactggaatggattgacttatttgaattacttgccagtggagctacatagagcacaattattgtatttaaattaccctttatgatcttacaaaacttgacagtaagatcatattgctaaagaaaccacatatttgaatcagggaacatggtgatatctagttgttcttcaactggaaacttcatgctttctgcccagcattcatgttgctggaaagagcaatgtacactaccagtgtagaaattaaatcatcaatcttatcaagatgtggatcctataagttacaataaaaattagcctgataagatatccccaccagaagaatattcacataaatgctatgggagcaacaagctattttctaaattagctttaatcctattctacaagagagaatccatatctagaatagttatagggatcaagaacccatggcttgattggtcataggcccaatgggagatcctaatattattgttctacaaaatgaaaataactcctaatgacttgttgctgcagtaataagttagtatgttgctcaactctcacaagagaagttttgtcttacaataaatggcaattaaagcagccccacaagatttatatcataccgatctcctcatggcctatgcatctagaagctaggaaacaaagaggaccctaagagagacatacatggtccccctggagaaggggaagggggcaagacctccaaagctaattgggagcatgggggaggggagagggagttagaagaaagagaaggggataaaaggagggagaggaggacaagagagagaaggaagatctagtcaagagaagatagaggagagcaagaaaagagataccatagtagagggagccttgtatgtttaaatagaaaactggcactagggaattgtccaaagatccacaaggtccaactaataatctaagcaatagtcgagaggctaccttaaaagcctttctctgataatgagattgatgactaccttatataccatcctagagccttcatccagtagctgatggaagcagaagcagacatctacagctaaacactgagctagttgcagacagggaggagtgatgagcaaagtcaagaccaggctggagaaacacacagaaacagcagacctgaaaaaaatgttgcacatggaccccagactgatagctgggagtccagcataggacttttctagaaaccctgaatgaggatatcagtttggaggtctggttaatctatggggacactggtagtggatcaatatttatccctagttcatgactggaatttgggtacccattccacatggaggaattctctgtcagcctagacacatgggggaggttctaggtcctgctccaaataatgtgttagactttgaagaactcccttgagaagactcaccctccctggggagcagaaaggggatgggatgagggttggtgagggacaggagaggaggggagggtgagggaactgggattgacaagtaaatgatgcttgtttctaatttaaatgaataaaggaaaagtaaaagaagaaaagaaaacaggccaaaagattataaaagacagaggtggtgggtgactataaagaaacactattatctaaataaaaacatgtcagaagcacacatgaacttatagtgtttatgaaagtatgtataataactacataatctcaagccaagaaaaaaatatcatctttcagtgatgaaggtgattttatttctcccagaattaaagccaaagacctaatgaaagtaattatcttcaaaaggttgaaaatacatactttgcaatacacagatctgcctagaaatctcatgttcacaatacacatgatgctcaattgaattccattcaatgttacagtttagataaacagtttgtagataaactcacaatgtatcatttctttttattttttgaccaaacagcttctcatctgttattcagaataattcctcgatggcaggatatccatcccaattgggggaaggggagaatttgaagaaaacctagaccacatacatatttgccattgggaaacaaagtctaaaatgatgttgttcacatcttctctactagtcctctccccgtcccaaagaaccttggtatatgtgcctcattttacagagagaggaaagcaggaactgagcatcccttacttgccatcctcaacccaaaatttgcatcattgctcagctctgcccttctcatatgacagttacaagtcaaggcttccaaagtccctctgtcatgtttggtgtcaatagtttatacagatgacttcatgtcttcatatctaatgtcttatatagattaatattaaacaatgttatttctctaaccacattttaaattaatttaaaaatccattaattgtgtctataaaatgcagacagagtgctgagacacaatataagcctgatgatctgaatttgaaactcacacccaccacatggagaatcaacttccaaaaattttcctattacttccacacttacaccattgtacaaacacaataataatgaacaaaatgaaatgaaataaaaaattaagtctctgtaggtaatgctactgtgcagcaaaagtaaaaatggcagcttaagcttgctttatggttacactttaccatcttccattaattataaggacttcaatcatggcagaactatgctgttattgtctcagtgtaacctaaccaggtgttccagatgttcttaatgtggacacctaaactatttgatatttgggttaagatctttccctctttcagaagaaacctcaggacagagggaatcttgtcttttaattttgagtctgtagactttttccatttcaaatatacatgaaacaagtgatgaagaaaattaatcaaaaggtgggaattgcaatgatattaggttcaatattaagcttcaatattatcatggaatcgcctgttatacactgagtgtttggcaataagggatttttagaagaaggagtttttattctcaacaggttccttaagtttagctcaaataaatctaagcaatccactctagaattaaatagtttcc

SEQ ID NO: 12 - DNA, 14931 bp, Cricetulus griseus
  source 1..14931: mol_type = genomic DNA
catgtacacttatgcaagtatgatatggcccaacacagtattttacaccaatttttatctataaaatatacatgtacatcaaaatatattattaataataacatcattattctttctttccaagtaataaacacatacactgaaattttggttcttgtggataattttaatgaaacaggaaatgcaaatttatcttagcatgtttacttcactttctttgcatagataaccagtaatcacattgatggatcatgtagtgaaatgtatttttaggtatctaaggaattttggcttcgttttgtgcttgttgacactgaattctattcctaacaacagtgtgtaaggattctgtctgatttcttttaccagtatttgtccatttgcattttctttattattcatggctgctgttctagaaagtggaaggtagtgtgtcaagtctgtttaacatgtttccctgatgatcagtgtcttaacacctctctgagtacatgttggccaatgtcgtttctagacccatctattcttgcttgacttatcctggtacatgcctgccaagaaatttctcctcatcctttctgtctcttcactgatttacttgatgtgtggatttcacattgatcatatggaaatagaagatacaattttctttattcacagtttggaagactttcaatctcatagatcatcattattttttgctactgttccctatgctatggtgaaatttccatttgaataattgcttaaacaattaacaagaaagaatctatttttacttgcaataacttccatttcagaacatttactacactgttactatatccaaaaactagttttatatatcatgtgagaaatgactaattcataatttggccatgacatttttttcagaaacagaaaaagtgaccaatacatacacaatgctataaatattaagacttcagcaaattaaatatttattcatgatatcacataaaattcatttattatgttttatttaaatgtgtttttaaaacagtggtatcactaaatattaagttagatgtgtttatgtgcttaatgaatttatattttagaatgttataagttgtatatagtcaaatatgtaataaattttattttttaggtctttctcattaaggtattttaattttgggtcccttttccagagtgactctagctcatgatgagttgacataaaaactaaacagtacaaaatgtacattgcattcagtattgcacttgatctttgcactgaagtttgagtcagttcatacatttagtacttgggaagtacattaagctaactttcattgctctggcaaaatgctcgataagataagagtctattgtggaaagccatggcagcaggaaagtaagactgctgatgatgtttaatccatagtcaagacgcagaaggagatgaatgctggtatccaacattttttgctgttcattttctctagaaccctagtccataaagatgtatgacttgcattcaaaatgcgtccccttcagttgttcaacttttctgtaaatatcctttcaggcatgtctagaagattgtttcgcaaatacttctcaatccattcaagttgatagtgcagattaatcactgcagaataaaagcctgtaacttggctcacgtgccaaggaatatgcacactcctgacacatcaataagtaaatcaaagtgtagcttttgcctttaacattgccagacttatgtaatgttctgcacgttcttcctccatcactttttattctaatggtgtttccttgacattgaatcacgctgtggaagctgcttagaattaacattgaaatctactgatatatttatgatgcagcaatttagatttactattttacttagaattttttataattgagagaatataatattttcacagttatctatctgctgtaaatagaggattttaaaaaaaatctctataacttttttttacaacacacagtaaaattaagttaaaatttaataaagtcactatgttgatttcaaagtgtgctacgcccacggtggtcacgcaggtgtagcagaagatgccactaaggtgggctaaggccgatgggttggggtctgcgctccctggagatgagccccaggcggttccctggcaatcagctgcgatcatgatgcccgatgagccannnnnnnnnnnnnnnnnnnnnnnnnnnnnnnnnnnnnnnnnnnnnnnnnnnnnnnnnnnnnnnnctgggtgactttatggaaagaatttgatagatttcatgatgtagaagaattttattaggcttattttacaggagactaagaccctgggacctaaagatatctgggtcctgagaatcaggaaatgggtagagacgtggttgatggtatgagacagattttagagaactcttagatcatgggcaatgaccgcaatctgatgcttagaatagatcatctataaacaattatgctgttctttttctttctgttgtatgatctgatgatgtagcccccttgccaagttccctgatcccccttgccaagttccctgattgtaacagtatataagcattgcttgagagcatattcaactacattgagtgtgtctgtctgtcatttcctcgccgattcctgatttctccttgagccttttcccttgttctccctcggtcggtggtctccacgagaggcggtccgtggcaaaagtgtataaatgttctaaaacatttgaactctaaaacatgcaaaatgaaaaattaaaataaataaacatgaaaattaaaatatattagctgctaaaagttaaacaatactatataatattttgttattagaattcaaaatcacattagttggatttaatttgaacattgcattctttcaataataatttcaataaaaaaagtttccccatgatagtagaaaataataacatatgtatctatctatttatttaactacacatatatagcatttgtttcaactaaaataaatgaatgagcaaagcacctaagtaattggtgtctattatatttatgaagccaatagtttcaaataaattatcatgcataaggaggtattgcaaatgttaaaccttttttgaaacagatattcccagttacagaaattataatttctaatctttcctataagtagaatgatgataattaatataggccatttgtaaataatgttcagattaaaatattctctatttcactagagaagaatgatattaaatgtattatattttatttcccattttgtttgcaccactattctatatccctcagcagtttaaatttgtttcaccatatgtgtgtgtgtttgtatcttaaatatggcactaaaattagaataatttaatataaatctttaggagaaaagatattgaattattttatgttgataggaaaatatcttttaattgtccaagaatactttttcttctattttaggactgatcagacccaggactaatattttatatgtactaattctatgtaccaaaatatgttattatctcatgaattctgtctcaatattgaggtaataaaaatagtccatcatgaactttaaaattaaaataatgattaattaatttttattcatattttgtttgtatgaatggttatacatcacatgtgtgcctggtgactgtgaatgtcaggagaaggtatgaaagccactggaattggaataagagataatatttgagatgttatgtgggtgctgagaattagacgcaagccatcttcaagaatagccagcatactataccactgagtaatccattcatccctcaataattatctttgtagacagtaaatatatttctaaactataaatgaccagaaaaattaatgtattattaatgaagacattcatctcatgtgacacacttcacctgtctaaatcagtaacactctctccactaattaagattttctaagtgcatgacacttactatttctaaagctgtccaatgggggccagtccccagtcagcacccagtgagataatccatgaatgcatttatatcttaggaaaaattcttatctatgtagtatttagaacattttcatgtgaggggataaacaaggaagcacagatgctttctgatagaaactttctctttaattcatctagaaaaaaaaaacctctcaggaaaatctctcttgctctcctcccaatgctctattcagcatcttctccctacttaattctagatctttttctctatgcctccttgctgctgccctgctggctctgctctatgcctccccatgtcacttttctttgctatctcaccgttaccttctctgcctcactctctgccttcttctctgcttctcacatggccaggctctggacaattatagttatatgttacattctcataacacatgatatgtcacatagtttctctcaggctagggatatcacaatgactggccaatgagcaagtggccttgcatgtagctctaagttggtgatggttcccagacagtaagtagccatttggttgaaatttgaggttgggtagtacatgaagactgaattttcttcaaactctggccttgaaatagtaaaacaacacctatgaaaatgacgacctgtatttgtctttagaggcaaccacatattgtctgcagggcctgctttgaatttgctctgaagttagcttgtttgtgtaaaaggaagaatcctatatcagcctgagaaatgtaaaatatcctagcatttcaagtcatcaaaattatatggagagtataaatcatccttctgactattcatagtcatatttgtgtccaccaagtataaaacacactaccaaagggctgtggaaaaaatcgccataactgttcttattagggaggcatagcagtggtacctgaggaagttacagcaacaaccagtcatccagtcaataaccccatggctttgccacttggaggtacccaataatgtttggctttgccgagtaggactccaacaaattcagagggtcaatttttaaatgctggttgtcactgctgaacagtcccattgccctctgcataattccacaatggaaagctttttacactgattgccaatcattaaacagcctactcagcataaacaggtatgatattattctgcattttgttacattactagatgaattcctatttcttcctacaatagtggaactgaaaaaagatacacaatcatactacccctctactaatcttatgacttatatcatttcaattttcagaccataatgcaaactattgaccaaaacatgtgaagatgaaaaatagaaatgtagaataatattacatataaaaagaaaaggcggacttattttgttttatttcttagcatgcatagcaatacatgatttgaggtttatataataaagggacaataaatcttcaagaaacttacccctactgaattaaaatattaaagaaggtcacacatttactcaaatatattagactactgggcaaatagacatgaaaagtagagttaatattgaggtaggccttctgtgaaatgtctaaggaaattatgtttcatacagtgtgtaaccaagtgggaatcatatcagaaagcagtcaaaagcttatattacaagtaacagatgcttggttatatgacctcccagagcttgactgtctatacacaaaaagtggtgttaataaaactgtaatttgggctatgtttttttaaatggcttcaccaacatgaaaggaagggaatgagcatgtcatggatgcttagagattatgcttccagcaagaagaattgagctttggctcttattacagaaacatgacaaggtgtgagttttatttattagaaattatataatattttaagctggggactaaaaattttattgaaacaaacaggcaagggataggcatgtactagaagcaaaaataggatgtcaatgctgtaatgttattttttggaccaaaatagtatttcctatagaaatgacaatgatcttaggttattattcttcataaagatgacaagttcacaagatatcctagttcattaaaatcgttttagtcatttaatagagtgctgtgatagattacacaaaggaaagcacttacgatgagaaataatgatatccacaattattttcttaattcttagaaacattctattgttatatctcaatctcagaagccacttattgctttattattgaaacatatgaaattgtaagttatatattgtctatggtgacatttcaaagaacatgtgacgtacagtgtagcacagataaagaacataactgcagctgaatcagtaactaaacttacatacattaaatctgccatgttggcaacagtgtgtgcactaccaaaggatgtactaatgctcacgacactcccctatgtcaccctttgttcatcattacatcataggtctattttgtttgcttttgaaatctagaccaagtcttttgtgtctttccaagcacagagctcattaatttacctcatagacttgttaaacttcttctggttcatcaattgaatagaaatactcactactaattatgtgagaccctgccagtaccatagcacatggataatttttacataaaacatgcatacaagtaagattattcagactgaacatgaattttagagaaatcaggaaggagtatatgggagtggttggagtgagactagagaaatgtaattaaactataatctcaatacaaagatctactaagcaaaaaacatgaaacattgtcattcaagtgaaacatcagtcttcaaattggaaagatatttttactaggaaaatgtctggtagatggttattatctagaaaacacaaaaattagaaaacggtaaactttaataaaaagaataatacaatgagactacatgaaaagttcttaactaatgaaacaaatatcttgaaacttttttcttaaaagtttaatatcaataaccatcatggaaattcaaattaaaactatttacatattacccctgaaataataactaatacccaataaaaataatataaacaaaaaatggcaatgcatgccatcatggatttgggagagagaatgttcattgcagttctgaatggatactggtgccaccacggtgaaaatctctgtataggtccttccaaaagctgaaaatagacatatcacaagacctgccacacatttttcaagcaaatacccaaaggactctacctgactgcagagacactttctcataaaatattattgttgatctattcataatatctggaaaatagaaacagccaagatgcccatcaactgattaatagatgataaaattattgtacatttcagtgtaatattattcagtttttaagaaaaatgaaattatgtaataagcatgtaaatggatatatcttgaaacaaccattccccattatattacctaaacattgaaagtccaaaatcatatgatctttttagtggatctactaatcttttgctatatgtattttattgaactacccatggatgtgagataattggtaacaacagcacatgggagagcatgggatcattcaaggaagattagagagaatgcattttttaggagataatggaggagcaatagaaaggattaaatgaggttactgatgaaagtgatggttagagaaggcaatatgaggagggataactagcacttagggccttttgaaaaagacatagagaaaatactattgtagaaacttcctataattggtgtatagttatatacaccaaagagctcagatggagttaccctataatggaaatattaactactttttatcactgtgataaaacatcctgaacagagcaacatagattgggaagcatttactttggcttacagttctaacgggataaaaattcatgatgaaagaatgaatatgtcagcaaacagcagtagcaatggcctgagaagcaggtgagagctcacatcttgaagtgtaagaatgtagcagagagaacaaactgcaaatgaccagaaaatgcttttggatcagagcccatacccctctgactgacttctccagaaattctgaacaaataaaactccccaaacagagccataactgaaggtccagtgtctgagactactaggggtatttcttattcaaaccactacaatggggtggggggagcaatcctccaagtaggcactacacacagacaaataaaaactctagtaactggaatggattgacttatttgaattacttgccagtggagctacatagagcacaattattgtatttaaattaccctttatgatcttacaaaacttgacagtaagatcatattgctaaagaaaccacatatttgaatcagggaacatggtgatatctagttgttcttcaactggaaacttcatgctttctgcccagcattcatgttgctggaaagagcaatgtacactaccagtgtagaaattaaatcatcaatcttatcaagatgtggatcctataagttacaataaaaattagcctgataagatatccccaccagaagaatattcacataaatgctatgggagcaacaagctattttctaaattagctttaatcctattctacaagagagaatccatatctagaatagttatagggatcaagaacccatggcttgattggtcataggcccaatgggagatcctaatattattgttctacaaaatgaaaataactcctaatgacttgttgctgcagtaataagttagtatgttgctcaactctcacaagagaagttttgtcttacaataaatggcaattaaagcagccccacaagatttatatcataccgatctcctcatggcctatgcatctagaagctaggaaacaaagaggaccctaagagagacatacatggtccccctggagaaggggaagggggcaagacctccaaagctaattgggagcatgggggaggggagagggagttagaagaaagagaaggggataaaaggagggagaggaggacaagagagagaaggaagatctagtcaagagaagatagaggagagcaagaaaagagataccatagtagagggagccttgtatgtttaaatagaaaactggcactagggaattgtccaaagatccacaaggtccaactaataatctaagcaatagtcgagaggctaccttaaaagcctttctctgataatgagattgatgactaccttatataccatcctagagccttcatccagtagctgatggaagcagaagcagacatctacagctaaacactgagctagttgcagacagggaggagtgatgagcaaagtcaagaccaggctggagaaacacacagaaacagcagacctgaaaaaaatgttgcacatggaccccagactgatagctgggagtccagcataggacttttctagaaaccctgaatgaggatatcagtttggaggtctggttaatctatggggacactggtagtggatcaatatttatccctagttcatgactggaatttgggtacccattccacatggaggaattctctgtcagcctagacacatgggggaggttctaggtcctgctccaaataatgtgttagactttgaagaactcccttgagaagactcaccctccctggggagcagaaaggggatgggatgagggttggtgagggacaggagaggaggggagggtgagggaactgggattgacaagtaaatgatgcttgtttctaatttaaatgaataaaggaaaagtaaaagaagaaaagaaaacaggccaaaagattataaaagacagaggtggtgggtgactataaagaaacactattatctaaataaaaatatgtcagaagcacacatgaacttatagtgtttatgaaagtatgtataataactacataatctcaagccaagaaaaaaatatcatctttcagtgatgaaggtgattttatttctcccagaattaaagccaaagacctaatgaaagtaattatcttcaaaaggttgaaaatacatactttgcaatacacagatctgcctagaaatctcatgttcacaatacacatgatgctcaattgaattccattcaatgttacagtttagataaacagtttgtagataaactcacaatgtatcatttctttttattttttgaccaaacagcttctcatctgttattcagaataattcctcgatggcaggatatccatcccaattgggggaaggggagaatttgaagaaaacctagaccacatacatatttgccattgggaaacaaagtctaaaatgatgttgttcacatcttctctactagtcctctccccgtcccaaagaaccttggtatatgtgcctcattttacagagagaggaaagcaggaactgagcatcccttacttgccatcctcaacccaaaatttgcatcattgctcagctctgcccttctcatatgacagttacaagtcaaggcttccaaagtccctctgtcatgtttggtgtcaatagtttatacagatgacttcatgtcttcatatctaatgtcttatatagattaatattaaacaatgttatttctctaaccacattttaaattaatttaaaaatccattaattgtgtctataaaatgcagacagagtgctgagacacaatataagcctgatgatctgaatttgaaactcacacccaccacatggagaatcaacttccaaaaattttcctattacttccacacttacaccattgtacaaacacaataataatgaacaaaatgaaatgaaataaaaaattaagtctctgtaggtaatgctactgtgcagcaaaagtaaaaatggcagcttaagcttgctttatggttacactttaccatcttccattaattataaggacttcaatcatggcagaactatgctgttattgtctcagtgtaacctaaccaggtgttccagatgttcttaatgtggacacctaaactatttgatatttgggttaagatctttccctctttcagaagaaacctcaggacagagggaatcttgtcttttaattttgagtctgtagactttttccatttcaaatatacatgaaacaagtgatgaagaaaattaatcaaaaggtgggaattgcaatgatattaggttcaatattaagcttcaatattatcatggaatcgcctgttatacactgagtgtttggcaataagggatttttagaagaaggagtttttattctcaacaggttccttaagtttagctcaaataaatctaagcaatccactctagaattaaatagtttcctaagggcacagctatgaatagagctcaatttacatataaaattttgttcaccatttatgtcattccagttttcattagtacaaggaaaatacaaaatatttagatgtcaatatcaagtgaatagttcatctccttttttaatatatatcacctaaatcaccattttctcagaaaaatctggcctgaagttctgtctggaacttcaacatgaaaaatatgcacagcttgctattataaatcctagttgatttttaagattcatgtctggtgtctgactcagaggggccagaggctagacaaatattttttgaatcttcattgtgaagatttttaatgattattttaatataaataacaaagatgatggataatgtaactttgtacagttcatagacgctgaactactttgtgcttaaaatgttagttccctatcataaatgataggtgataagtgtatgtttaatactttccctctgagctatattcatgtactagagaattattttaaacatgaaaagactgtgtttatagtctcagctcctgagaactggtccaaccttaggcaggtgaatgccaggagcaacgtttttcttctacagaggatgctttgctgccaagcaacctggttgtgtggaaatgttccttttttaatcaagtttaaagggtcttcatcatgctgttgctccacatattttcaggttagagcttggtccttggagtattatcttttaccagaaaattcatagtattctttcaataactaacaactaaacttttcgataaaaaagaattggaatttcaattttaaagcctgagtaaaattcttgtgaatcaggatattttattttaagtcttatcttttaaaaagttattttattttttaaaaaattataatatactttcataatttccctccttcacttttctttacaaacacttctatagatcaccatgtgtttttttttttacatttatggcctctttctgttcattgttattacatacaaatagtcttgcctatagaagaacaccacaatttgttacctgataacaaattatcaacccttaaaacctacaaactattgatattactgaaaagactatacttatagatgtaaagatatatgtgtgtgcacatatatagatacacatatatgtaggatttttaattttagattttagacatcaaaattatttatatgactgagaaactagacactataaatgagcattcagtattcaacaccgtgattttagatattgtcacaatgacagaaaattttcttatagaaaattttaagttttgtgattgctctgtgcacttagtgaagtctcacagaaaaagaatcatagtatttttagtttataataaaaagtacatataattaaaatggttggcacaaaacaacatttgagcatttttcctatttactatcaagtagtatcattttgaaataataatttgactagtttcaaaaatgaaaacaaaatttaaactaaatgcctaatctagcctgataacatttttatgaatgaaattattcaatagtgttatcaattaggggcccaaaacttttcctaaaataaaacttttaatttttttccatttttatttaaattagaaacaaaattgttttacatgtaaatcagagtttcctcaccctccccttctccctgtccctcactaacaccctacttgtcccataccatttctgctccccagggagggtgaggccttccatggggaaacttcagagtctgtctatcctttcggatagggcctaggccctcacccatttgtctaggctaaggctcacaaagtttactcctatgctagtgataagtactgatctactacaagagacaccatagatttcctaggcttcctcactgacacccatgttcatggggtctggaacaatcatatgctagtttcctaggtatcagtctggggaccatgagctcccccttgttcaggtcaactgtttctgtgggtttcaccaccctggtcttgactgctttgctcatcactcctccctttctgtaactgggttccagtacaattccgtgtttagctgtgggtgtctacttctactttcatcagcttctgggatggagcctctaggatagcatacaattagtcatcatctcattatcagggaagggcatttaaagtagcctctccattgttgcttggattgttagttggtgtcatctttgtagatctctggacatttccctagtgccagatatctctttaaacctacaagactacctctattatggtatctcttttcttgctctcgtctattcttccagacaaaatcttcctgctcccttatattttcctctcccctcctcttctccccttctcattctcctagatccatcttcccttcccccatgctcccaagagagatgttgctcaggagatcttgttccttaacccttttcttggggatctgtctctcttagggttgtccttgtttcctagcttctctggaagtgtggattgtaagctggtaatcatttgctccatgtctaaaatccatatatgagtgatgtttgtctttttgtgactgggttacctcactcaaaatggtttcttccatatgtctgtggatttcaatagcacaaacaacatacagtatcttggggcaacactaaccaaacaagtgaaagaccagtatagcaagaactttgagtttaaagaaagaaattaaagaagataccagaaaatggaaagatctcccatgctctttgataggcagaatcaacatagtaaaaatggcaatcttgccaaaatccatctacagactcaatgcaatccccattaaataccagcacacttcttcacagacctgaaagaataatacttaactttatatggagaaacaaaagacccaggataggccaaacaaccctgtacaatgaaggcacttccagaggcatccccatccctgacttcaagctctattatagagtaataatcctgaaaacagcttggtaatggcacaaaaatagacaggtagaccaatggaattgagttgaaaaccctgatattaacccacatatctatgaacacctgactttgacaaagaagctaaggttatacaatgtaagaaagaaagcatcttcaacaaatcgtgctggcataactggatgctggcatgtagaagactgcagatagatccatgtctaatgccatgcacaaaacttaagtccaaatggatcaaaaacctcaacataaatccagccacactgaacctcatagaagagaaagtgggaagtatccttgaataaattggtacaggagaccacatcttgaacttaacaccagtagcacagacaatcagatcaataatcaataaatgggacctcctgaaactgagaagcttctgtaaggcaatggataagtcaacaggacaaaatggcagcccacggaatgggaaaagatattcaccaatcctatatctgacagagggctgctctctatttgcaaagaacacaataagctagtttttaaaacaccaattaatccgattataaagttgggtagagaactaaataaagaattgttaacagagcaatctaacttggcagaaagacacataagaaagtgctcaccat

SEQ ID NO: 13 - DNA, 23 bp, synthetic construct
  misc_feature 1..23: note = Synthetic: Guide Sequence for AAVS1- Like Sequence in CHO
  source 1..23: mol_type = other DNA
ccccgctggcgccgggatcgggg

SEQ ID NO: 14 - DNA, 23 bp, synthetic construct
  misc_feature 1..23: note = Synthetic: Guide Sequence for AAVS1- Like Sequence in CHO
  source 1..23: mol_type = other DNA
gagtcgagcaccgctcgggcagg

SEQ ID NO: 15 - DNA, 23 bp, synthetic construct
  misc_feature 1..23: note = Synthetic: Guide Sequence for AAVS1- Like Sequence in CHO
  source 1..23: mol_type = other DNA
ttccccgctggcgccgggatcgg

SEQ ID NO: 16 - DNA, 23 bp, synthetic construct
  misc_feature 1..23: note = Synthetic: Guide Sequence for AAVS1- Like Sequence in CHO
  source 1..23: mol_type = other DNA
gtgtgcggaagacgccgccgggg

SEQ ID NO: 17 - DNA, 23 bp, synthetic construct
  misc_feature 1..23: note = Synthetic: Guide Sequence for AAVS1- Like Sequence in CHO
  source 1..23: mol_type = other DNA
cggtgacagcgcggatgacaggg

SEQ ID NO: 18 - DNA, 23 bp, synthetic construct
  misc_feature 1..23: note = Synthetic: Guide Sequence for AAVS1- Like Sequence in CHO
  source 1..23: mol_type = other DNA
cagcgcggatgacaggggcgagg

SEQ ID NO: 19 - DNA, 23 bp, synthetic construct
  misc_feature 1..23: note = Synthetic: Guide Sequence for AAVS1- Like Sequence in CHO
  source 1..23: mol_type = other DNA
gccggcgtccgattccccgctgg

SEQ ID NO: 20 - DNA, 23 bp, synthetic construct
  misc_feature 1..23: note = Synthetic: Guide Sequence for AAVS1- Like Sequence in CHO
  source 1..23: mol_type = other DNA
cgtgtgcggaagacgccgccggg

SEQ ID NO: 21 - DNA, 23 bp, synthetic construct
  misc_feature 1..23: note = Synthetic: Guide Sequence for AAVS1- Like Sequence in CHO
  source 1..23: mol_type = other DNA
gaggcgctccaccgtctgttggg

SEQ ID NO: 22 - DNA, 23 bp, synthetic construct
  misc_feature 1..23: note = Synthetic: Guide Sequence for AAVS1- Like Sequence in CHO
  source 1..23: mol_type = other DNA
gtccgattccccgctggcgccgg

SEQ ID NO: 23 - DNA, 23 bp, synthetic construct
  misc_feature 1..23: note = Synthetic: Guide Sequence for AAVS1- Like Sequence in CHO
  source 1..23: mol_type = other DNA
gaccccgggggccccgatcccgg

SEQ ID NO: 24 - DNA, 23 bp, synthetic construct
  misc_feature 1..23: note = Synthetic: Guide Sequence for AAVS1- Like Sequence in CHO
  source 1..23: mol_type = other DNA
cggcgtcttccgcacacggatgg

SEQ ID NO: 25 - DNA, 23 bp, synthetic construct
  misc_feature 1..23: note = Synthetic: Guide Sequence for AAVS1- Like Sequence in CHO
  source 1..23: mol_type = other DNA
tcgagcaccgctcgggcaggcgg

SEQ ID NO: 26 - DNA, 23 bp, synthetic construct
  misc_feature 1..23: note = Synthetic: Guide Sequence for AAVS1- Like Sequence in CHO
  source 1..23: mol_type = other DNA
agctcacgccggccccataaagg

SEQ ID NO: 27 - DNA, 23 bp, synthetic construct
  misc_feature 1..23: note = Synthetic: Guide Sequence for AAVS1- Like Sequence in CHO
  source 1..23: mol_type = other DNA
catcgtcctctatatatagcagg

SEQ ID NO: 28 - DNA, 23 bp, synthetic construct
  misc_feature 1..23: note = Synthetic: Guide Sequence for AAVS1- Like Sequence in CHO
  source 1..23: mol_type = other DNA
agaggcgctccaccgtctgttgg

SEQ ID NO: 29 - DNA, 23 bp, synthetic construct
  misc_feature 1..23: note = Synthetic: Guide Sequence for AAVS1- Like Sequence in CHO
  source 1..23: mol_type = other DNA
ggtcggctgcgcgaagcatcagg

SEQ ID NO: 30 - DNA, 23 bp, synthetic construct
  misc_feature 1..23: note = Synthetic: Guide Sequence for AAVS1- Like Sequence in CHO
  source 1..23: mol_type = other DNA
tgcttcgcgcagccgaccccggg

SEQ ID NO: 31 - DNA, 23 bp, synthetic construct
  misc_feature 1..23: note = Synthetic: Guide Sequence for AAVS1- Like Sequence in CHO
  source 1..23: mol_type = other DNA
ggccccgatcccggcgccagcgg

SEQ ID NO: 32 - DNA, 23 bp, synthetic construct
  misc_feature 1..23: note = Synthetic: Guide Sequence for AAVS1- Like Sequence in CHO
  source 1..23: mol_type = other DNA
tccgattccccgctggcgccggg

SEQ ID NO: 33 - DNA, 23 bp, synthetic construct
  misc_feature 1..23: note = Synthetic: Guide Sequence for AAVS1- Like Sequence in CHO
  source 1..23: mol_type = other DNA
tcccggcgccagcggggaatcgg

SEQ ID NO: 34 - DNA, 23 bp, synthetic construct
  misc_feature 1..23: note = Synthetic: Guide Sequence for AAVS1- Like Sequence in CHO
  source 1..23: mol_type = other DNA
tggtggagtcgagcaccgctcgg

SEQ ID NO: 35 - DNA, 23 bp, synthetic construct
  misc_feature 1..23: note = Synthetic: Guide Sequence for AAVS1- Like Sequence in CHO
  source 1..23: mol_type = other DNA
caagatggtcctcactctcgggg

SEQ ID NO: 36 - DNA, 23 bp, synthetic construct
  misc_feature 1..23: note = Synthetic: Guide Sequence for AAVS1- Like Sequence in CHO
  source 1..23: mol_type = other DNA
gcttcgcgcagccgaccccgggg

SEQ ID NO: 37 - DNA, 23 bp, synthetic construct
  misc_feature 1..23: note = Synthetic: Guide Sequence for AAVS1- Like Sequence in CHO
  source 1..23: mol_type = other DNA
gtggagcgcctcttctccagggg

SEQ ID NO: 38 - DNA, 23 bp, synthetic construct
  misc_feature 1..23: note = Synthetic: Guide Sequence for AAVS1- Like Sequence in CHO
  source 1..23: mol_type = other DNA
gacgtgtcagccttccaggtggg

SEQ ID NO: 39 - DNA, 23 bp, synthetic construct
  misc_feature 1..23: note = Synthetic: Guide Sequence for AAVS1- Like Sequence in CHO
  source 1..23: mol_type = other DNA
gccagcggggaatcggacgccgg

SEQ ID NO: 40 - DNA, 23 bp, synthetic construct
  misc_feature 1..23: note = Synthetic: Guide Sequence for AAVS1- Like Sequence in CHO
  source 1..23: mol_type = other DNA
tctccccgtcatccaaaagctgg

SEQ ID NO: 41 - DNA, 23 bp, synthetic construct
  misc_feature 1..23: note = Synthetic: Guide Sequence for AAVS1- Like Sequence in CHO
  source 1..23: mol_type = other DNA
ggtggagtcgagcaccgctcggg

SEQ ID NO: 42 - DNA, 23 bp, synthetic construct
  misc_feature 1..23: note = Synthetic: Guide Sequence for AAVS1- Like Sequence in CHO
  source 1..23: mol_type = other DNA
gctgcccaaatatagtccatggg

SEQ ID NO: 43 - DNA, 23 bp, synthetic construct
  misc_feature 1..23: note = Synthetic: Guide Sequence for AAVS1- Like Sequence in CHO
  source 1..23: mol_type = other DNA
atgcttcgcgcagccgaccccgg

SEQ ID NO: 44 - DNA, 23 bp, synthetic construct
  misc_feature 1..23: note = Synthetic: Guide Sequence for AAVS1- Like Sequence in CHO
  source 1..23: mol_type = other DNA
gcggtgacagcgcggatgacagg

SEQ ID NO: 45 - DNA, 23 bp, synthetic construct
  misc_feature 1..23: note = Synthetic: Guide Sequence for AAVS1- Like Sequence in CHO
  source 1..23: mol_type = other DNA
atgctcgggggccgctgacctgg

SEQ ID NO: 46 - DNA, 23 bp, synthetic construct
  misc_feature 1..23: note = Synthetic: Guide Sequence for AAVS1- Like Sequence in CHO
  source 1..23: mol_type = other DNA
ttgtattgccgggatccttctgg

SEQ ID NO: 47 - DNA, 23 bp, synthetic construct
  misc_feature 1..23: note = Synthetic: Guide Sequence for AAVS1- Like Sequence in CHO
  source 1..23: mol_type = other DNA
tccccgctggcgccgggatcggg

SEQ ID NO: 48 - DNA, 23 bp, synthetic construct
  misc_feature 1..23: note = Synthetic: Guide Sequence for AAVS1- Like Sequence in CHO
  source 1..23: mol_type = other DNA
ctcgactccaccaacgccgacgg

SEQ ID NO: 49 - DNA, 23 bp, synthetic construct
  misc_feature 1..23: note = Synthetic: Guide Sequence for AAVS1- Like Sequence in CHO
  source 1..23: mol_type = other DNA
ggtggcaagatcaccaaaagggg

SEQ ID NO: 50 - DNA, 23 bp, synthetic construct
  misc_feature 1..23: note = Synthetic: Guide Sequence for AAVS1- Like Sequence in CHO
  source 1..23: mol_type = other DNA
ggcgctgatgccgtcggcgttgg

SEQ ID NO: 51 - DNA, 23 bp, synthetic construct
  misc_feature 1..23: note = Synthetic: Guide Sequence for AAVS1- Like Sequence in CHO
  source 1..23: mol_type = other DNA
tccacgagcatcctagcaagagg

SEQ ID NO: 52 - DNA, 23 bp, synthetic construct
  misc_feature 1..23: note = Synthetic: Guide Sequence for AAVS1- Like Sequence in CHO
  source 1..23: mol_type = other DNA
aggctgacacgtcaggcctgagg

SEQ ID NO: 53 - DNA, 23 bp, synthetic construct
  misc_feature 1..23: note = Synthetic: Guide Sequence for AAVS1- Like Sequence in CHO
  source 1..23: mol_type = other DNA
gggtgtaagccatccgtgtgcgg

SEQ ID NO: 54 - DNA, 23 bp, synthetic construct
  misc_feature 1..23: note = Synthetic: Guide Sequence for AAVS1- Like Sequence in CHO
  source 1..23: mol_type = other DNA
aggatcccggcaatacaagatgg

SEQ ID NO: 55 - DNA, 23 bp, synthetic construct
  misc_feature 1..23: note = Synthetic: Guide Sequence for AAVS1- Like Sequence in CHO
  source 1..23: mol_type = other DNA
ggatggggcccaacagacggtgg

SEQ ID NO: 56 - DNA, 23 bp, synthetic construct
  misc_feature 1..23: note = Synthetic: Guide Sequence for AAVS1- Like Sequence in CHO
  source 1..23: mol_type = other DNA
gaggaccatcttgtattgccggg

SEQ ID NO: 57 - DNA, 23 bp, synthetic construct
  misc_feature 1..23: note = Synthetic: Guide Sequence for AAVS1- Like Sequence in CHO
  source 1..23: mol_type = other DNA
agtcgcccagggtcctggtgggg

SEQ ID NO: 58 - DNA, 23 bp, synthetic construct
  misc_feature 1..23: note = Synthetic: Guide Sequence for AAVS1- Like Sequence in CHO
  source 1..23: mol_type = other DNA
ggcgggaggagagtcccacctgg

SEQ ID NO: 59 - DNA, 23 bp, synthetic construct
  misc_feature 1..23: note = Synthetic: Guide Sequence for AAVS1- Like Sequence in CHO
  source 1..23: mol_type = other DNA
acctaccccaccaggaccctggg

SEQ ID NO: 60 - DNA, 23 bp, synthetic construct
  misc_feature 1..23: note = Synthetic: Guide Sequence for AAVS1- Like Sequence in CHO
  source 1..23: mol_type = other DNA
tcagcgtctttgaccagtccagg

SEQ ID NO: 61 - DNA, 23 bp, synthetic construct
  misc_feature 1..23: note = Synthetic: Guide Sequence for AAVS1- Like Sequence in CHO
  source 1..23: mol_type = other DNA
cgtcccgccgcctgcccgagcgg

SEQ ID NO: 62 - DNA, 23 bp, synthetic construct
  misc_feature 1..23: note = Synthetic: Guide Sequence for AAVS1- Like Sequence in CHO
  source 1..23: mol_type = other DNA
gtccccgggatccccggggtcgg

SEQ ID NO: 63 - DNA, 23 bp, synthetic construct
  misc_feature 1..23: note = Synthetic: Guide Sequence for AAVS1- Like Sequence in CHO
  source 1..23: mol_type = other DNA
agcaccgctcgggcaggcggcgg

SEQ ID NO: 64 - DNA, 23 bp, synthetic construct
  misc_feature 1..23: note = Synthetic: Guide Sequence for AAVS1- Like Sequence in CHO
  source 1..23: mol_type = other DNA
cggtggagcgcctcttctccagg

SEQ ID NO: 65 - DNA, 23 bp, synthetic construct
  misc_feature 1..23: note = Synthetic: Guide Sequence for AAVS1- Like Sequence in CHO
  source 1..23: mol_type = other DNA
gccccgatcccggcgccagcggg

SEQ ID NO: 66 - DNA, 23 bp, synthetic construct
  misc_feature 1..23: note = Synthetic: Guide Sequence for AAVS1- Like Sequence in CHO
  source 1..23: mol_type = other DNA
atcggggcccccggggtccccgg

SEQ ID NO: 67 - DNA, 23 bp, synthetic construct
  misc_feature 1..23: note = Synthetic: Guide Sequence for AAVS1- Like Sequence in CHO
  source 1..23: mol_type = other DNA
attctcggctcatccccttttgg

SEQ ID NO: 68 - DNA, 23 bp, synthetic construct
  misc_feature 1..23: note = Synthetic: Guide Sequence for AAVS1- Like Sequence in CHO
  source 1..23: mol_type = other DNA
accaccccatggactatatttgg

SEQ ID NO: 69 - DNA, 23 bp, synthetic construct
  misc_feature 1..23: note = Synthetic: Guide Sequence for AAVS1- Like Sequence in CHO
  source 1..23: mol_type = other DNA
tagcaagaggacgacaacccagg

SEQ ID NO: 70 - DNA, 23 bp, synthetic construct
  misc_feature 1..23: note = Synthetic: Guide Sequence for AAVS1- Like Sequence in CHO
  source 1..23: mol_type = other DNA
gctgatgccgtcggcgttggtgg

SEQ ID NO: 71 - DNA, 23 bp, synthetic construct
  misc_feature 1..23: note = Synthetic: Guide Sequence for AAVS1- Like Sequence in CHO
  source 1..23: mol_type = other DNA
tacaagatggtcctcactctcgg

SEQ ID NO: 72 - DNA, 23 bp, synthetic construct
  misc_feature 1..23: note = Synthetic: Guide Sequence for AAVS1- Like Sequence in CHO
  source 1..23: mol_type = other DNA
tgccgggatccttctggattcgg

SEQ ID NO: 73 - DNA, 23 bp, synthetic construct
  misc_feature 1..23: note = Synthetic: Guide Sequence for AAVS1- Like Sequence in CHO
  source 1..23: mol_type = other DNA
cccaaatatagtccatggggtgg

SEQ ID NO: 74 - DNA, 23 bp, synthetic construct
  misc_feature 1..23: note = Synthetic: Guide Sequence for AAVS1- Like Sequence in CHO
  source 1..23: mol_type = other DNA
tacctgtagaatgggaccagtgg

SEQ ID NO: 75 - DNA, 23 bp, synthetic construct
  misc_feature 1..23: note = Synthetic: Guide Sequence for AAVS1- Like Sequence in CHO
  source 1..23: mol_type = other DNA
tcttgctaggatgctcgtggagg

SEQ ID NO: 76 - DNA, 23 bp, synthetic construct
  misc_feature 1..23: note = Synthetic: Guide Sequence for AAVS1- Like Sequence in CHO
  source 1..23: mol_type = other DNA
atgccagcttttggatgacgggg

SEQ ID NO: 77 - DNA, 23 bp, synthetic construct
  misc_feature 1..23: note = Synthetic: Guide Sequence for AAVS1- Like Sequence in CHO
  source 1..23: mol_type = other DNA
atagtccatggggtggtaggtgg

SEQ ID NO: 78 - DNA, 23 bp, synthetic construct
  misc_feature 1..23: note = Synthetic: Guide Sequence for AAVS1- Like Sequence in CHO
  source 1..23: mol_type = other DNA
caggaccctgctatatatagagg

SEQ ID NO: 79 - DNA, 23 bp, synthetic construct
  misc_feature 1..23: note = Synthetic: Guide Sequence for AAVS1- Like Sequence in CHO
  source 1..23: mol_type = other DNA
ggggccggcgtgagctgtgtggg

SEQ ID NO: 80 - DNA, 23 bp, synthetic construct
  misc_feature 1..23: note = Synthetic: Guide Sequence for AAVS1- Like Sequence in CHO
  source 1..23: mol_type = other DNA
acctggaaggctgacacgtcagg

SEQ ID NO: 81 - DNA, 23 bp, synthetic construct
  misc_feature 1..23: note = Synthetic: Guide Sequence for AAVS1- Like Sequence in CHO
  source 1..23: mol_type = other DNA
cagcggacagcacgggtcacagg

SEQ ID NO: 82 - DNA, 23 bp, synthetic construct
  misc_feature 1..23: note = Synthetic: Guide Sequence for AAVS1- Like Sequence in CHO
  source 1..23: mol_type = other DNA
cgccgggatcggggcccccgggg

SEQ ID NO: 83 - DNA, 23 bp, synthetic construct
  misc_feature 1..23: note = Synthetic: Guide Sequence for AAVS1- Like Sequence in CHO
  source 1..23: mol_type = other DNA
gcgccgggatcggggcccccggg

SEQ ID NO: 84 - DNA, 23 bp, synthetic construct
  misc_feature 1..23: note = Synthetic: Guide Sequence for AAVS1- Like Sequence in CHO
  source 1..23: mol_type = other DNA
gccgggatccttctggattcggg

SEQ ID NO: 85 - DNA, 23 bp, synthetic construct
  misc_feature 1..23: note = Synthetic: Guide Sequence for AAVS1- Like Sequence in CHO
  source 1..23: mol_type = other DNA
gctgtcaccgctctccccggcgg

SEQ ID NO: 86 - DNA, 23 bp, synthetic construct
  misc_feature 1..23: note = Synthetic: Guide Sequence for AAVS1- Like Sequence in CHO
  source 1..23: mol_type = other DNA
ggtgacagcgcggatgacagggg

SEQ ID NO: 87 - DNA, 23 bp, synthetic construct
  misc_feature 1..23: note = Synthetic: Guide Sequence for AAVS1- Like Sequence in CHO
  source 1..23: mol_type = other DNA
gaagacgccgccggggagagcgg

SEQ ID NO: 88 - DNA, 23 bp, synthetic construct
  misc_feature 1..23: note = Synthetic: Guide Sequence for AAVS1- Like Sequence in CHO
  source 1..23: mol_type = other DNA
aaggggtacactgccttggaggg

SEQ ID NO: 89 - DNA, 23 bp, synthetic construct
  misc_feature 1..23: note = Synthetic: Guide Sequence for AAVS1- Like Sequence in CHO
  source 1..23: mol_type = other DNA
cttctagaacctaccccaccagg

SEQ ID NO: 90 - DNA, 23 bp, synthetic construct
  misc_feature 1..23: note = Synthetic: Guide Sequence for AAVS1- Like Sequence in CHO
  source 1..23: mol_type = other DNA
ggggccgctgacctggtgcaggg

SEQ ID NO: 91 - DNA, 23 bp, synthetic construct
  misc_feature 1..23: note = Synthetic: Guide Sequence for AAVS1- Like Sequence in CHO
  source 1..23: mol_type = other DNA
tcccgaatccagaaggatcccgg

SEQ ID NO: 92 - DNA, 23 bp, synthetic construct
  misc_feature 1..23: note = Synthetic: Guide Sequence for AAVS1- Like Sequence in CHO
  source 1..23: mol_type = other DNA
tcacagtgtctgagtcgcccagg

SEQ ID NO: 93 - DNA, 23 bp, synthetic construct
  misc_feature 1..23: note = Synthetic: Guide Sequence for AAVS1- Like Sequence in CHO
  source 1..23: mol_type = other DNA
gctaggatgctcgtggaggtggg

SEQ ID NO: 94 - DNA, 23 bp, synthetic construct
  misc_feature 1..23: note = Synthetic: Guide Sequence for AAVS1- Like Sequence in CHO
  source 1..23: mol_type = other DNA
ctcgtggaggtggggaataaagg

SEQ ID NO: 95 - DNA, 23 bp, synthetic construct
  misc_feature 1..23: note = Synthetic: Guide Sequence for AAVS1- Like Sequence in CHO
  source 1..23: mol_type = other DNA
tgatctgtgccccgagagtgagg

SEQ ID NO: 96 - DNA, 23 bp, synthetic construct
  misc_feature 1..23: note = Synthetic: Guide Sequence for AAVS1- Like Sequence in CHO
  source 1..23: mol_type = other DNA
tgaattaataggacatggggagg

SEQ ID NO: 97 - DNA, 23 bp, synthetic construct
  misc_feature 1..23: note = Synthetic: Guide Sequence for AAVS1- Like Sequence in CHO
  source 1..23: mol_type = other DNA
actctcggggcacagatcactgg

SEQ ID NO: 98 - DNA, 23 bp, synthetic construct
  misc_feature 1..23: note = Synthetic: Guide Sequence for AAVS1- Like Sequence in CHO
  source 1..23: mol_type = other DNA
gaccactggtcccattctacagg

SEQ ID NO: 99 - DNA, 23 bp, synthetic construct
  misc_feature 1..23: note = Synthetic: Guide Sequence for AAVS1- Like Sequence in CHO
  source 1..23: mol_type = other DNA
tgaggaccatcttgtattgccgg

SEQ ID NO: 100 - DNA, 23 bp, synthetic construct
  misc_feature 1..23: note = Synthetic: Guide Sequence for AAVS1- Like Sequence in CHO
  source 1..23: mol_type = other DNA
agaatccgtctgtcctgggctgg

SEQ ID NO: 101 - DNA, 23 bp, synthetic construct
  misc_feature 1..23: note = Synthetic: Guide Sequence for AAVS1- Like Sequence in CHO
  source 1..23: mol_type = other DNA
tgacgtgtcagccttccaggtgg

SEQ ID NO: 102 - DNA, 23 bp, synthetic construct
  misc_feature 1..23: note = Synthetic: Guide Sequence for AAVS1- Like Sequence in CHO
  source 1..23: mol_type = other DNA
aaaagcatcccgaatccagaagg

SEQ ID NO: 103 - DNA, 23 bp, synthetic construct
  misc_feature 1..23: note = Synthetic: Guide Sequence for AAVS1- Like Sequence in CHO
  source 1..23: mol_type = other DNA
ttttcccgaggccacactcaggg

SEQ ID NO: 104 - DNA, 23 bp, synthetic construct
  misc_feature 1..23: note = Synthetic: Guide Sequence for AAVS1- Like Sequence in CHO
  source 1..23: mol_type = other DNA
agcgccctgcaccaggtcagcgg

SEQ ID NO: 105 - DNA, 23 bp, synthetic construct
  misc_feature 1..23: note = Synthetic: Guide Sequence for AAVS1- Like Sequence in CHO
  source 1..23: mol_type = other DNA
tgagtcgcccagggtcctggtgg

SEQ ID NO: 106 - DNA, 23 bp, synthetic construct
  misc_feature 1..23: note = Synthetic: Guide Sequence for AAVS1- Like Sequence in CHO
  source 1..23: mol_type = other DNA
cacgtcaggcctgaggtcacagg

SEQ ID NO: 107 - DNA, 23 bp, synthetic construct
  misc_feature 1..23: note = Synthetic: Guide Sequence for AAVS1- Like Sequence in CHO
  source 1..23: mol_type = other DNA
gttttcccgaggccacactcagg

SEQ ID NO: 108 - DNA, 23 bp, synthetic construct
  misc_feature 1..23: note = Synthetic: Guide Sequence for AAVS1- Like Sequence in CHO
  source 1..23: mol_type = other DNA
gagtcgcccagggtcctggtggg

SEQ ID NO: 109 - DNA, 23 bp, synthetic construct
  misc_feature 1..23: note = Synthetic: Guide Sequence for AAVS1- Like Sequence in CHO
  source 1..23: mol_type = other DNA
gaatccgtctgtcctgggctggg

SEQ ID NO: 110 - DNA, 23 bp, synthetic construct
  misc_feature 1..23: note = Synthetic: Guide Sequence for AAVS1- Like Sequence in CHO
  source 1..23: mol_type = other DNA
ccggggagggaggatgctcgggg

SEQ ID NO: 111 - DNA, 23 bp, synthetic construct
  misc_feature 1..23: note = Synthetic: Guide Sequence for AAVS1- Like Sequence in CHO
  source 1..23: mol_type = other DNA
gagccgagaattgaattaatagg

SEQ ID NO: 112 - DNA, 23 bp, synthetic construct
  misc_feature 1..23: note = Synthetic: Guide Sequence for AAVS1- Like Sequence in CHO
  source 1..23: mol_type = other DNA
gtgacccgtgctgtccgctgtgg

SEQ ID NO: 113 - DNA, 23 bp, synthetic construct
  misc_feature 1..23: note = Synthetic: Guide Sequence for AAVS1- Like Sequence in CHO
  source 1..23: mol_type = other DNA
gggggcgtcaagtcagagctggg

SEQ ID NO: 114 - DNA, 23 bp, synthetic construct
  misc_feature 1..23: note = Synthetic: Guide Sequence for AAVS1- Like Sequence in CHO
  source 1..23: mol_type = other DNA
cgcgctgtcaccgctctccccgg

SEQ ID NO: 115 - DNA, 23 bp, synthetic construct
  misc_feature 1..23: note = Synthetic: Guide Sequence for AAVS1- Like Sequence in CHO
  source 1..23: mol_type = other DNA
tgctgcccaaatatagtccatgg

SEQ ID NO: 116 - DNA, 23 bp, synthetic construct
  misc_feature 1..23: note = Synthetic: Guide Sequence for AAVS1- Like Sequence in CHO
  source 1..23: mol_type = other DNA
agttgaggagaaacctatggggg

SEQ ID NO: 117 - DNA, 23 bp, synthetic construct
  misc_feature 1..23: note = Synthetic: Guide Sequence for AAVS1- Like Sequence in CHO
  source 1..23: mol_type = other DNA
atcgtcctctatatatagcaggg

SEQ ID NO: 118 - DNA, 23 bp, synthetic construct
  misc_feature 1..23: note = Synthetic: Guide Sequence for AAVS1- Like Sequence in CHO
  source 1..23: mol_type = other DNA
gagttgaggagaaacctatgggg

SEQ ID NO: 119 - DNA, 23 bp, synthetic construct
  misc_feature 1..23: note = Synthetic: Guide Sequence for AAVS1- Like Sequence in CHO
  source 1..23: mol_type = other DNA
aggggtacactgccttggagggg

SEQ ID NO: 120 - DNA, 23 bp, synthetic construct
  misc_feature 1..23: note = Synthetic: Guide Sequence for AAVS1- Like Sequence in CHO
  source 1..23: mol_type = other DNA
atagagtccctctggggacaggg

SEQ ID NO: 121 - DNA, 23 bp, synthetic construct
  misc_feature 1..23: note = Synthetic: Guide Sequence for AAVS1- Like Sequence in CHO
  source 1..23: mol_type = other DNA
ctaggatgctcgtggaggtgggg

SEQ ID NO: 122 - DNA, 23 bp, synthetic construct
  misc_feature 1..23: note = Synthetic: Guide Sequence for AAVS1- Like Sequence in CHO
  source 1..23: mol_type = other DNA
cgcggatgacaggggcgaggcgg

SEQ ID NO: 123 - DNA, 23 bp, synthetic construct
  misc_feature 1..23: note = Synthetic: Guide Sequence for AAVS1- Like Sequence in CHO
  source 1..23: mol_type = other DNA
aaaacagaatccgtctgtcctgg

SEQ ID NO: 124 - DNA, 23 bp, synthetic construct
  misc_feature 1..23: note = Synthetic: Guide Sequence for AAVS1- Like Sequence in CHO
  source 1..23: mol_type = other DNA
tgctaggatgctcgtggaggtgg

SEQ ID NO: 125 - DNA, 23 bp, synthetic construct
  misc_feature 1..23: note = Synthetic: Guide Sequence for AAVS1- Like Sequence in CHO
  source 1..23: mol_type = other DNA
gagtgtggcctcgggaaaacagg

SEQ ID NO: 126 - DNA, 23 bp, synthetic construct
  misc_feature 1..23: note = Synthetic: Guide Sequence for AAVS1- Like Sequence in CHO
  source 1..23: mol_type = other DNA
cgaggcggcccctgcagggcagg

SEQ ID NO: 127 - DNA, 23 bp, synthetic construct
  misc_feature 1..23: note = Synthetic: Guide Sequence for AAVS1- Like Sequence in CHO
  source 1..23: mol_type = other DNA
cttacaccccgtgcctttccagg

SEQ ID NO: 128 - DNA, 23 bp, synthetic construct
  misc_feature 1..23: note = Synthetic: Guide Sequence for AAVS1- Like Sequence in CHO
  source 1..23: mol_type = other DNA
ctgcccaaatatagtccatgggg

SEQ ID NO: 129 - DNA, 23 bp, synthetic construct
  misc_feature 1..23: note = Synthetic: Guide Sequence for AAVS1- Like Sequence in CHO
  source 1..23: mol_type = other DNA
aggggcaaaggaccctcctgagg

SEQ ID NO: 130 - DNA, 23 bp, synthetic construct
  misc_feature 1..23: note = Synthetic: Guide Sequence for AAVS1- Like Sequence in CHO
  source 1..23: mol_type = other DNA
tctcaccatagagtccctctggg

SEQ ID NO: 131 - DNA, 23 bp, synthetic construct
  misc_feature 1..23: note = Synthetic: Guide Sequence for AAVS1- Like Sequence in CHO
  source 1..23: mol_type = other DNA
agtgtacccctttgttcccctgg

SEQ ID NO: 132 - DNA, 23 bp, synthetic construct
  misc_feature 1..23: note = Synthetic: Guide Sequence for AAVS1- Like Sequence in CHO
  source 1..23: mol_type = other DNA
gttgaggagaaacctatgggggg

SEQ ID NO: 133 - DNA, 23 bp, synthetic construct
  misc_feature 1..23: note = Synthetic: Guide Sequence for AAVS1- Like Sequence in CHO
  source 1..23: mol_type = other DNA
cacagtgtctgagtcgcccaggg

SEQ ID NO: 134 - DNA, 23 bp, synthetic construct
  misc_feature 1..23: note = Synthetic: Guide Sequence for AAVS1- Like Sequence in CHO
  source 1..23: mol_type = other DNA
tcctcttgctaggatgctcgtgg

SEQ ID NO: 135 - DNA, 23 bp, synthetic construct
  misc_feature 1..23: note = Synthetic: Guide Sequence for AAVS1- Like Sequence in CHO
  source 1..23: mol_type = other DNA
cacatgatcaccaaagtccctgg

SEQ ID NO: 136 - DNA, 23 bp, synthetic construct
  misc_feature 1..23: note = Synthetic: Guide Sequence for AAVS1- Like Sequence in CHO
  source 1..23: mol_type = other DNA
aaacagaatccgtctgtcctggg

SEQ ID NO: 137 - DNA, 23 bp, synthetic construct
  misc_feature 1..23: note = Synthetic: Guide Sequence for AAVS1- Like Sequence in CHO
  source 1..23: mol_type = other DNA
gtgcagggcgctgatgccgtcgg

SEQ ID NO: 138 - DNA, 23 bp, synthetic construct
  misc_feature 1..23: note = Synthetic: Guide Sequence for AAVS1- Like Sequence in CHO
  source 1..23: mol_type = other DNA
cagcacggactttttttgtttgg

SEQ ID NO: 139 - DNA, 23 bp, synthetic construct
  misc_feature 1..23: note = Synthetic: Guide Sequence for AAVS1- Like Sequence in CHO
  source 1..23: mol_type = other DNA
cagccgaccccggggatcccggg

SEQ ID NO: 140 - DNA, 23 bp, synthetic construct
  misc_feature 1..23: note = Synthetic: Guide Sequence for AAVS1- Like Sequence in CHO
  source 1..23: mol_type = other DNA
tttggtcaagattttgcaactgg

SEQ ID NO: 141 - DNA, 23 bp, synthetic construct
  misc_feature 1..23: note = Synthetic: Guide Sequence for AAVS1- Like Sequence in CHO
  source 1..23: mol_type = other DNA
ttggtcaagattttgcaactggg

SEQ ID NO: 142 - DNA, 23 bp, synthetic construct
  misc_feature 1..23: note = Synthetic: Guide Sequence for AAVS1- Like Sequence in CHO
  source 1..23: mol_type = other DNA
cctgtcagagaggatgctctagg

SEQ ID NO: 143 - DNA, 23 bp, synthetic construct
  misc_feature 1..23: note = Synthetic: Guide Sequence for AAVS1- Like Sequence in CHO
  source 1..23: mol_type = other DNA
tgggttgtcgtcctcttgctagg

SEQ ID NO: 144 - DNA, 23 bp, synthetic construct
  misc_feature 1..23: note = Synthetic: Guide Sequence for AAVS1- Like Sequence in CHO
  source 1..23: mol_type = other DNA
ctcaccatagagtccctctgggg

SEQ ID NO: 145 - DNA, 23 bp, synthetic construct
  misc_feature 1..23: note = Synthetic: Guide Sequence for AAVS1- Like Sequence in CHO
  source 1..23: mol_type = other DNA
agacgctgacaccctgagtgtgg

SEQ ID NO: 146 - DNA, 23 bp, synthetic construct
  misc_feature 1..23: note = Synthetic: Guide Sequence for AAVS1- Like Sequence in CHO
  source 1..23: mol_type = other DNA
ggtggagcgcctcttctccaggg

SEQ ID NO: 147 - DNA, 23 bp, synthetic construct
  misc_feature 1..23: note = Synthetic: Guide Sequence for AAVS1- Like Sequence in CHO
  source 1..23: mol_type = other DNA
aacaaaggggtacactgccttgg

SEQ ID NO: 148 - DNA, 23 bp, synthetic construct
  misc_feature 1..23: note = Synthetic: Guide Sequence for AAVS1- Like Sequence in CHO
  source 1..23: mol_type = other DNA
ctgtccccagagggactctatgg

SEQ ID NO: 149 - DNA, 23 bp, synthetic construct
  misc_feature 1..23: note = Synthetic: Guide Sequence for AAVS1- Like Sequence in CHO
  source 1..23: mol_type = other DNA
gtctctgaccccctcatttgtgg

SEQ ID NO: 150 - DNA, 23 bp, synthetic construct
  misc_feature 1..23: note = Synthetic: Guide Sequence for AAVS1- Like Sequence in CHO
  source 1..23: mol_type = other DNA
gtctgagtcgcccagggtcctgg

SEQ ID NO: 151 - DNA, 23 bp, synthetic construct
  misc_feature 1..23: note = Synthetic: Guide Sequence for AAVS1- Like Sequence in CHO
  source 1..23: mol_type = other DNA
gcctgacgtgtcagccttccagg

SEQ ID NO: 152 - DNA, 23 bp, synthetic construct
  misc_feature 1..23: note = Synthetic: Guide Sequence for AAVS1- Like Sequence in CHO
  source 1..23: mol_type = other DNA
atgtcccacacagctcacgccgg

SEQ ID NO: 153 - DNA, 23 bp, synthetic construct
  misc_feature 1..23: note = Synthetic: Guide Sequence for AAVS1- Like Sequence in CHO
  source 1..23: mol_type = other DNA
ggctgtgccgcaggcttccaggg

SEQ ID NO: 154 - DNA, 23 bp, synthetic construct
  misc_feature 1..23: note = Synthetic: Guide Sequence for AAVS1- Like Sequence in CHO
  source 1..23: mol_type = other DNA
caccccgtgcctttccaggctgg

SEQ ID NO: 155 - DNA, 23 bp, synthetic construct
  misc_feature 1..23: note = Synthetic: Guide Sequence for AAVS1- Like Sequence in CHO
  source 1..23: mol_type = other DNA
tcaccaaggtgtctgcatggcgg

SEQ ID NO: 156 - DNA, 23 bp, synthetic construct
  misc_feature 1..23: note = Synthetic: Guide Sequence for AAVS1- Like Sequence in CHO
  source 1..23: mol_type = other DNA
gaattgaattaataggacatggg

SEQ ID NO: 157 - DNA, 23 bp, synthetic construct
  misc_feature 1..23: note = Synthetic: Guide Sequence for AAVS1- Like Sequence in CHO
  source 1..23: mol_type = other DNA
aatatagtccatggggtggtagg

SEQ ID NO: 158 - DNA, 23 bp, synthetic construct
  misc_feature 1..23: note = Synthetic: Guide Sequence for AAVS1- Like Sequence in CHO
  source 1..23: mol_type = other DNA
cagggcatagtttttaaagcagg

SEQ ID NO: 159 - DNA, 23 bp, synthetic construct
  misc_feature 1..23: note = Synthetic: Guide Sequence for AAVS1- Like Sequence in CHO
  source 1..23: mol_type = other DNA
cggcatcagcgccctgcaccagg

SEQ ID NO: 160 - DNA, 23 bp, synthetic construct
  misc_feature 1..23: note = Synthetic: Guide Sequence for AAVS1- Like Sequence in CHO
  source 1..23: mol_type = other DNA
gcagccgaccccggggatcccgg

SEQ ID NO: 161 - DNA, 23 bp, synthetic construct
  misc_feature 1..23: note = Synthetic: Guide Sequence for AAVS1- Like Sequence in CHO
  source 1..23: mol_type = other DNA
acaggacctgtatttgaggttgg

SEQ ID NO: 162 - DNA, 23 bp, synthetic construct
  misc_feature 1..23: note = Synthetic: Guide Sequence for AAVS1- Like Sequence in CHO
  source 1..23: mol_type = other DNA
ggggcgaggcggcccctgcaggg

SEQ ID NO: 163 - DNA, 23 bp, synthetic construct
  misc_feature 1..23: note = Synthetic: Guide Sequence for AAVS1- Like Sequence in CHO
  source 1..23: mol_type = other DNA
aacctaccccaccaggaccctgg

SEQ ID NO: 164 - DNA, 23 bp, synthetic construct
  misc_feature 1..23: note = Synthetic: Guide Sequence for AAVS1- Like Sequence in CHO
  source 1..23: mol_type = other DNA
gcaccgctcgggcaggcggcggg

SEQ ID NO: 165 - DNA, 23 bp, synthetic construct
  misc_feature 1..23: note = Synthetic: Guide Sequence for AAVS1- Like Sequence in CHO
  source 1..23: mol_type = other DNA
tggaagcctgcggcacagccagg

SEQ ID NO: 166 - DNA, 23 bp, synthetic construct
  misc_feature 1..23: note = Synthetic: Guide Sequence for AAVS1- Like Sequence in CHO
  source 1..23: mol_type = other DNA
gaaccaacactgtggccaggagg

SEQ ID NO: 167 - DNA, 23 bp, synthetic construct
  misc_feature 1..23: note = Synthetic: Guide Sequence for AAVS1- Like Sequence in CHO
  source 1..23: mol_type = other DNA
agcagggtcctgttttcccgagg

SEQ ID NO: 168 - DNA, 23 bp, synthetic construct
  misc_feature 1..23: note = Synthetic: Guide Sequence for AAVS1- Like Sequence in CHO
  source 1..23: mol_type = other DNA
tgggtggcaagatcaccaaaagg

SEQ ID NO: 169 - DNA, 23 bp, synthetic construct
  misc_feature 1..23: note = Synthetic: Guide Sequence for AAVS1- Like Sequence in CHO
  source 1..23: mol_type = other DNA
agccgaccccggggatcccgggg

SEQ ID NO: 170 - DNA, 23 bp, synthetic construct
  misc_feature 1..23: note = Synthetic: Guide Sequence for AAVS1- Like Sequence in CHO
  source 1..23: mol_type = other DNA
catggcaacttccatctcctggg

SEQ ID NO: 171 - DNA, 23 bp, synthetic construct
  misc_feature 1..23: note = Synthetic: Guide Sequence for AAVS1- Like Sequence in CHO
  source 1..23: mol_type = other DNA
acagcacgggtcacaggaagtgg

SEQ ID NO: 172 - DNA, 23 bp, synthetic construct
  misc_feature 1..23: note = Synthetic: Guide Sequence for AAVS1- Like Sequence in CHO
  source 1..23: mol_type = other DNA
gggggccgctgacctggtgcagg

SEQ ID NO: 173 - DNA, 23 bp, synthetic construct
  misc_feature 1..23: note = Synthetic: Guide Sequence for AAVS1- Like Sequence in CHO
  source 1..23: mol_type = other DNA
aggggcgaggcggcccctgcagg

SEQ ID NO: 174 - DNA, 23 bp, synthetic construct
  misc_feature 1..23: note = Synthetic: Guide Sequence for AAVS1- Like Sequence in CHO
  source 1..23: mol_type = other DNA
gtggtcacccctgtccccagagg

SEQ ID NO: 175 - DNA, 23 bp, synthetic construct
  misc_feature 1..23: note = Synthetic: Guide Sequence for AAVS1- Like Sequence in CHO
  source 1..23: mol_type = other DNA
tggggccggcgtgagctgtgtgg

SEQ ID NO: 176 - DNA, 23 bp, synthetic construct
  misc_feature 1..23: note = Synthetic: Guide Sequence for AAVS1- Like Sequence in CHO
  source 1..23: mol_type = other DNA
ggaagcctgcggcacagccaggg

SEQ ID NO: 177 - DNA, 23 bp, synthetic construct
  misc_feature 1..23: note = Synthetic: Guide Sequence for AAVS1- Like Sequence in CHO
  source 1..23: mol_type = other DNA
cctgagctgatctcctggactgg

SEQ ID NO: 178 - DNA, 23 bp, synthetic construct
  misc_feature 1..23: note = Synthetic: Guide Sequence for AAVS1- Like Sequence in CHO
  source 1..23: mol_type = other DNA
atccaaaagctggcattgtcagg

SEQ ID NO: 179 - DNA, 23 bp, synthetic construct
  misc_feature 1..23: note = Synthetic: Guide Sequence for AAVS1- Like Sequence in CHO
  source 1..23: mol_type = other DNA
ctctcaccatagagtccctctgg

SEQ ID NO: 180 - DNA, 23 bp, synthetic construct
  misc_feature 1..23: note = Synthetic: Guide Sequence for AAVS1- Like Sequence in CHO
  source 1..23: mol_type = other DNA
ggggggcgtcaagtcagagctgg

SEQ ID NO: 181 - DNA, 23 bp, synthetic construct
  misc_feature 1..23: note = Synthetic: Guide Sequence for AAVS1- Like Sequence in CHO
  source 1..23: mol_type = other DNA
gggtggaaatctaagagacaggg

SEQ ID NO: 182 - DNA, 23 bp, synthetic construct
  misc_feature 1..23: note = Synthetic: Guide Sequence for AAVS1- Like Sequence in CHO
  source 1..23: mol_type = other DNA
cggggagagcggtgacagcgcgg

SEQ ID NO: 183 - DNA, 23 bp, synthetic construct
  misc_feature 1..23: note = Synthetic: Guide Sequence for AAVS1- Like Sequence in CHO
  source 1..23: mol_type = other DNA
agaattgaattaataggacatgg

SEQ ID NO: 184 - DNA, 23 bp, synthetic construct
  misc_feature 1..23: note = Synthetic: Guide Sequence for AAVS1- Like Sequence in CHO
  source 1..23: mol_type = other DNA
ccataaaggaagttttccacagg

SEQ ID NO: 185 - DNA, 23 bp, synthetic construct
  misc_feature 1..23: note = Synthetic: Guide Sequence for AAVS1- Like Sequence in CHO
  source 1..23: mol_type = other DNA
agtgaaccaacactgtggccagg

SEQ ID NO: 186 - DNA, 23 bp, synthetic construct
  misc_feature 1..23: note = Synthetic: Guide Sequence for AAVS1- Like Sequence in CHO
  source 1..23: mol_type = other DNA
gttgggagggaactcttgggagg

SEQ ID NO: 187 - DNA, 23 bp, synthetic construct
  misc_feature 1..23: note = Synthetic: Guide Sequence for AAVS1- Like Sequence in CHO
  source 1..23: mol_type = other DNA
cgggtcacaggaagtggggtagg

SEQ ID NO: 188 - DNA, 23 bp, synthetic construct
  misc_feature 1..23: note = Synthetic: Guide Sequence for AAVS1- Like Sequence in CHO
  source 1..23: mol_type = other DNA
ggcctggctagcctcagaggagg

SEQ ID NO: 189 - DNA, 23 bp, synthetic construct
  misc_feature 1..23: note = Synthetic: Guide Sequence for AAVS1- Like Sequence in CHO
  source 1..23: mol_type = other DNA
ggtaggttctagaaggtgacagg

SEQ ID NO: 190 - DNA, 23 bp, synthetic construct
  misc_feature 1..23: note = Synthetic: Guide Sequence for AAVS1- Like Sequence in CHO
  source 1..23: mol_type = other DNA
acaagatggtcctcactctcggg

SEQ ID NO: 191 - DNA, 23 bp, synthetic construct
  misc_feature 1..23: note = Synthetic: Guide Sequence for AAVS1- Like Sequence in CHO
  source 1..23: mol_type = other DNA
caaggtgtctgcatggcgggagg

SEQ ID NO: 192 - DNA, 23 bp, synthetic construct
  misc_feature 1..23: note = Synthetic: Guide Sequence for AAVS1- Like Sequence in CHO
  source 1..23: mol_type = other DNA
tgtttcactcatccaggcagagg

SEQ ID NO: 193 - DNA, 23 bp, synthetic construct
  misc_feature 1..23: note = Synthetic: Guide Sequence for AAVS1- Like Sequence in CHO
  source 1..23: mol_type = other DNA
tgtggaaaacttcctttatgggg

SEQ ID NO: 194 - DNA, 23 bp, synthetic construct
  misc_feature 1..23: note = Synthetic: Guide Sequence for AAVS1- Like Sequence in CHO
  source 1..23: mol_type = other DNA
gaggacgacaacccaggagatgg

SEQ ID NO: 195 - DNA, 23 bp, synthetic construct
  misc_feature 1..23: note = Synthetic: Guide Sequence for AAVS1- Like Sequence in CHO
  source 1..23: mol_type = other DNA
gggtggcaagatcaccaaaaggg

SEQ ID NO: 196 - DNA, 23 bp, synthetic construct
  misc_feature 1..23: note = Synthetic: Guide Sequence for AAVS1- Like Sequence in CHO
  source 1..23: mol_type = other DNA
ctggtggggtaggttctagaagg

SEQ ID NO: 197 - DNA, 23 bp, synthetic construct
  misc_feature 1..23: note = Synthetic: Guide Sequence for AAVS1- Like Sequence in CHO
  source 1..23: mol_type = other DNA
actcttcaggcctttgcaggagg

SEQ ID NO: 198 - DNA, 23 bp, synthetic construct
  misc_feature 1..23: note = Synthetic: Guide Sequence for AAVS1- Like Sequence in CHO
  source 1..23: mol_type = other DNA
cagagggactctatggtgagagg

SEQ ID NO: 199 - DNA, 23 bp, synthetic construct
  misc_feature 1..23: note = Synthetic: Guide Sequence for AAVS1- Like Sequence in CHO
  source 1..23: mol_type = other DNA
agcacgggtcacaggaagtgggg

SEQ ID NO: 200 - DNA, 23 bp, synthetic construct
  misc_feature 1..23: note = Synthetic: Guide Sequence for AAVS1- Like Sequence in CHO
  source 1..23: mol_type = other DNA
ctatggtgagaggcgtcctgtgg

SEQ ID NO: 201 - DNA, 23 bp, synthetic construct
  misc_feature 1..23: note = Synthetic: Guide Sequence for AAVS1- Like Sequence in CHO
  source 1..23: mol_type = other DNA
cagcacgggtcacaggaagtggg

SEQ ID NO: 202 - DNA, 23 bp, synthetic construct
  misc_feature 1..23: note = Synthetic: Guide Sequence for AAVS1- Like Sequence in CHO
  source 1..23: mol_type = other DNA
atgggatggggcccaacagacgg

SEQ ID NO: 203 - DNA, 23 bp, synthetic construct
  misc_feature 1..23: note = Synthetic: Guide Sequence for AAVS1- Like Sequence in CHO
  source 1..23: mol_type = other DNA
ctcccgccatgcagacaccttgg

SEQ ID NO: 204 - DNA, 23 bp, synthetic construct
  misc_feature 1..23: note = Synthetic: Guide Sequence for AAVS1- Like Sequence in CHO
  source 1..23: mol_type = other DNA
ggggatcccggggaccccggggg

SEQ ID NO: 205 - DNA, 23 bp, synthetic construct
  misc_feature 1..23: note = Synthetic: Guide Sequence for AAVS1- Like Sequence in CHO
  source 1..23: mol_type = other DNA
tgtccgctgtggcctcaggaggg

SEQ ID NO: 206 - DNA, 23 bp, synthetic construct
  misc_feature 1..23: note = Synthetic: Guide Sequence for AAVS1- Like Sequence in CHO
  source 1..23: mol_type = other DNA
gcagttgggagggaactcttggg

SEQ ID NO: 207 - DNA, 23 bp, synthetic construct
  misc_feature 1..23: note = Synthetic: Guide Sequence for AAVS1- Like Sequence in CHO
  source 1..23: mol_type = other DNA
gcctggctagcctcagaggaggg

SEQ ID NO: 208 - DNA, 23 bp, synthetic construct
  misc_feature 1..23: note = Synthetic: Guide Sequence for AAVS1- Like Sequence in CHO
  source 1..23: mol_type = other DNA
gagagttgaggagaaacctatgg

SEQ ID NO: 209 - DNA, 23 bp, synthetic construct
  misc_feature 1..23: note = Synthetic: Guide Sequence for AAVS1- Like Sequence in CHO
  source 1..23: mol_type = other DNA
acctgtatttgaggttggcctgg

SEQ ID NO: 210 - DNA, 23 bp, synthetic construct
  misc_feature 1..23: note = Synthetic: Guide Sequence for AAVS1- Like Sequence in CHO
  source 1..23: mol_type = other DNA
tcgggcaggcggcgggacgccgg

SEQ ID NO: 211 - DNA, 23 bp, synthetic construct
  misc_feature 1..23: note = Synthetic: Guide Sequence for AAVS1- Like Sequence in CHO
  source 1..23: mol_type = other DNA
tagagtccctctggggacagggg

SEQ ID NO: 212 - DNA, 23 bp, synthetic construct
  misc_feature 1..23: note = Synthetic: Guide Sequence for AAVS1- Like Sequence in CHO
  source 1..23: mol_type = other DNA
gaagtgacactgaagggcctggg

SEQ ID NO: 213 - DNA, 23 bp, synthetic construct
  misc_feature 1..23: note = Synthetic: Guide Sequence for AAVS1- Like Sequence in CHO
  source 1..23: mol_type = other DNA
agcagcctgagctgatctcctgg

SEQ ID NO: 214 - DNA, 23 bp, synthetic construct
  misc_feature 1..23: note = Synthetic: Guide Sequence for AAVS1- Like Sequence in CHO
  source 1..23: mol_type = other DNA
tcatggcaacttccatctcctgg

SEQ ID NO: 215 - DNA, 23 bp, synthetic construct
  misc_feature 1..23: note = Synthetic: Guide Sequence for AAVS1- Like Sequence in CHO
  source 1..23: mol_type = other DNA
gtcacaggttcctgtcagagagg

SEQ ID NO: 216 - DNA, 23 bp, synthetic construct
  misc_feature 1..23: note = Synthetic: Guide Sequence for AAVS1- Like Sequence in CHO
  source 1..23: mol_type = other DNA
caccaaggtgtctgcatggcggg

SEQ ID NO: 217 - DNA, 23 bp, synthetic construct
  misc_feature 1..23: note = Synthetic: Guide Sequence for AAVS1- Like Sequence in CHO
  source 1..23: mol_type = other DNA
gtaggttctagaaggtgacaggg

SEQ ID NO: 218 - DNA, 23 bp, synthetic construct
  misc_feature 1..23: note = Synthetic: Guide Sequence for AAVS1- Like Sequence in CHO
  source 1..23: mol_type = other DNA
aattgaattaataggacatgggg

SEQ ID NO: 219 - DNA, 23 bp, synthetic construct
  misc_feature 1..23: note = Synthetic: Guide Sequence for AAVS1- Like Sequence in CHO
  source 1..23: mol_type = other DNA
ttccaagcacctgatttctgtgg

SEQ ID NO: 220 - DNA, 23 bp, synthetic construct
  misc_feature 1..23: note = Synthetic: Guide Sequence for AAVS1- Like Sequence in CHO
  source 1..23: mol_type = other DNA
ctgtcagagaggatgctctaggg

SEQ ID NO: 221 - DNA, 23 bp, synthetic construct
  misc_feature 1..23: note = Synthetic: Guide Sequence for AAVS1- Like Sequence in CHO
  source 1..23: mol_type = other DNA
gtgctgtccgctgtggcctcagg

SEQ ID NO: 222 - DNA, 23 bp, synthetic construct
  misc_feature 1..23: note = Synthetic: Guide Sequence for AAVS1- Like Sequence in CHO
  source 1..23: mol_type = other DNA
cccggggagggaggatgctcggg

SEQ ID NO: 223 - DNA, 23 bp, synthetic construct
  misc_feature 1..23: note = Synthetic: Guide Sequence for AAVS1- Like Sequence in CHO
  source 1..23: mol_type = other DNA
ggggtggctcggggggccccggg

SEQ ID NO: 224 - DNA, 23 bp, synthetic construct
  misc_feature 1..23: note = Synthetic: Guide Sequence for AAVS1- Like Sequence in CHO
  source 1..23: mol_type = other DNA
gtcaagtcagagctgggccctgg

SEQ ID NO: 225 - DNA, 23 bp, synthetic construct
  misc_feature 1..23: note = Synthetic: Guide Sequence for AAVS1- Like Sequence in CHO
  source 1..23: mol_type = other DNA
gacactgaagggcctgggcctgg

SEQ ID NO: 226 - DNA, 23 bp, synthetic construct
  misc_feature 1..23: note = Synthetic: Guide Sequence for AAVS1- Like Sequence in CHO
  source 1..23: mol_type = other DNA
ctgaaagtgaaccaacactgtgg

SEQ ID NO: 227 - DNA, 23 bp, synthetic construct
  misc_feature 1..23: note = Synthetic: Guide Sequence for AAVS1- Like Sequence in CHO
  source 1..23: mol_type = other DNA
aaaggggtacactgccttggagg

SEQ ID NO: 228 - DNA, 23 bp, synthetic construct
  misc_feature 1..23: note = Synthetic: Guide Sequence for AAVS1- Like Sequence in CHO
  source 1..23: mol_type = other DNA
tctggaaacttctaagcattcgg

SEQ ID NO: 229 - DNA, 23 bp, synthetic construct
  misc_feature 1..23: note = Synthetic: Guide Sequence for AAVS1- Like Sequence in CHO
  source 1..23: mol_type = other DNA
agagttgaggagaaacctatggg

SEQ ID NO: 230 - DNA, 23 bp, synthetic construct
  misc_feature 1..23: note = Synthetic: Guide Sequence for AAVS1- Like Sequence in CHO
  source 1..23: mol_type = other DNA
ctctgaggctagccaggcccagg

SEQ ID NO: 231 - DNA, 23 bp, synthetic construct
  misc_feature 1..23: note = Synthetic: Guide Sequence for AAVS1- Like Sequence in CHO
  source 1..23: mol_type = other DNA
aggggtggctcggggggccccgg

SEQ ID NO: 232 - DNA, 23 bp, synthetic construct
  misc_feature 1..23: note = Synthetic: Guide Sequence for AAVS1- Like Sequence in CHO
  source 1..23: mol_type = other DNA
tccacattgatttgcctttctgg

SEQ ID NO: 233 - DNA, 23 bp, synthetic construct
  misc_feature 1..23: note = Synthetic: Guide Sequence for AAVS1- Like Sequence in CHO
  source 1..23: mol_type = other DNA
agggtggaaatctaagagacagg

SEQ ID NO: 234 - DNA, 23 bp, synthetic construct
  misc_feature 1..23: note = Synthetic: Guide Sequence for AAVS1- Like Sequence in CHO
  source 1..23: mol_type = other DNA
ctgtccgctgtggcctcaggagg

SEQ ID NO: 235 - DNA, 23 bp, synthetic construct
  misc_feature 1..23: note = Synthetic: Guide Sequence for AAVS1- Like Sequence in CHO
  source 1..23: mol_type = other DNA
agacacaggacctgtatttgagg

SEQ ID NO: 236 - DNA, 23 bp, synthetic construct
  misc_feature 1..23: note = Synthetic: Guide Sequence for AAVS1- Like Sequence in CHO
  source 1..23: mol_type = other DNA
gggtggctcggggggccccgggg

SEQ ID NO: 237 - DNA, 23 bp, synthetic construct
  misc_feature 1..23: note = Synthetic: Guide Sequence for AAVS1- Like Sequence in CHO
  source 1..23: mol_type = other DNA
tggctgtgccgcaggcttccagg

SEQ ID NO: 238 - DNA, 23 bp, synthetic construct
  misc_feature 1..23: note = Synthetic: Guide Sequence for AAVS1- Like Sequence in CHO
  source 1..23: mol_type = other DNA
tccagaaaggcaaatcaatgtgg

SEQ ID NO: 239 - DNA, 23 bp, synthetic construct
  misc_feature 1..23: note = Synthetic: Guide Sequence for AAVS1- Like Sequence in CHO
  source 1..23: mol_type = other DNA
caccagcctggaaaggcacgggg

SEQ ID NO: 240 - DNA, 23 bp, synthetic construct
  misc_feature 1..23: note = Synthetic: Guide Sequence for AAVS1- Like Sequence in CHO
  source 1..23: mol_type = other DNA
cggggtccccgggatccccgggg

SEQ ID NO: 241 - DNA, 23 bp, synthetic construct
  misc_feature 1..23: note = Synthetic: Guide Sequence for AAVS1- Like Sequence in CHO
  source 1..23: mol_type = other DNA
acactgccttggaggggcaaagg

SEQ ID NO: 242 - DNA, 23 bp, synthetic construct
  misc_feature 1..23: note = Synthetic: Guide Sequence for AAVS1- Like Sequence in CHO
  source 1..23: mol_type = other DNA
tgacaggaacctgtgacctcagg

SEQ ID NO: 243 - DNA, 23 bp, synthetic construct
  misc_feature 1..23: note = Synthetic: Guide Sequence for AAVS1- Like Sequence in CHO
  source 1..23: mol_type = other DNA
tctcatgtgggctatcaagatgg

SEQ ID NO: 244 - DNA, 23 bp, synthetic construct
  misc_feature 1..23: note = Synthetic: Guide Sequence for AAVS1- Like Sequence in CHO
  source 1..23: mol_type = other DNA
gggaactcttgggagggccaggg

SEQ ID NO: 245 - DNA, 23 bp, synthetic construct
  misc_feature 1..23: note = Synthetic: Guide Sequence for AAVS1- Like Sequence in CHO
  source 1..23: mol_type = other DNA
gaggccacagcggacagcacggg

SEQ ID NO: 246 - DNA, 23 bp, synthetic construct
  misc_feature 1..23: note = Synthetic: Guide Sequence for AAVS1- Like Sequence in CHO
  source 1..23: mol_type = other DNA
tgtcctattaattcaattctcgg

SEQ ID NO: 247 - DNA, 23 bp, synthetic construct
  misc_feature 1..23: note = Synthetic: Guide Sequence for AAVS1- Like Sequence in CHO
  source 1..23: mol_type = other DNA
agggaactcttgggagggccagg

SEQ ID NO: 248 - DNA, 23 bp, synthetic construct
  misc_feature 1..23: note = Synthetic: Guide Sequence for AAVS1- Like Sequence in CHO
  source 1..23: mol_type = other DNA
gcacctgatttctgtggtattgg

SEQ ID NO: 249 - DNA, 23 bp, synthetic construct
  misc_feature 1..23: note = Synthetic: Guide Sequence for AAVS1- Like Sequence in CHO
  source 1..23: mol_type = other DNA
gtatcttgagtgtcttttctcgg

SEQ ID NO: 250 - DNA, 23 bp, synthetic construct
  misc_feature 1..23: note = Synthetic: Guide Sequence for AAVS1- Like Sequence in CHO
  source 1..23: mol_type = other DNA
ctgtggaaaacttcctttatggg

SEQ ID NO: 251 - DNA, 23 bp, synthetic construct
  misc_feature 1..23: note = Synthetic: Guide Sequence for AAVS1- Like Sequence in CHO
  source 1..23: mol_type = other DNA
gggacctccagcagatgcagagg

SEQ ID NO: 252 - DNA, 23 bp, synthetic construct
  misc_feature 1..23: note = Synthetic: Guide Sequence for AAVS1- Like Sequence in CHO
  source 1..23: mol_type = other DNA
ctggggacaggggtgaccactgg

SEQ ID NO: 253 - DNA, 23 bp, synthetic construct
  misc_feature 1..23: note = Synthetic: Guide Sequence for AAVS1- Like Sequence in CHO
  source 1..23: mol_type = other DNA
ttcagtgtcacttcttttggggg

SEQ ID NO: 254 - DNA, 23 bp, synthetic construct
  misc_feature 1..23: note = Synthetic: Guide Sequence for AAVS1- Like Sequence in CHO
  source 1..23: mol_type = other DNA
tccctcctctgaggctagccagg

SEQ ID NO: 255 - DNA, 23 bp, synthetic construct
  misc_feature 1..23: note = Synthetic: Guide Sequence for AAVS1- Like Sequence in CHO
  source 1..23: mol_type = other DNA
ggcgccgggatcggggcccccgg

SEQ ID NO: 256 - DNA, 23 bp, synthetic construct
  misc_feature 1..23: note = Synthetic: Guide Sequence for AAVS1- Like Sequence in CHO
  source 1..23: mol_type = other DNA
ggttctagaaggtgacagggtgg

SEQ ID NO: 257 - DNA, 23 bp, synthetic construct
  misc_feature 1..23: note = Synthetic: Guide Sequence for AAVS1- Like Sequence in CHO
  source 1..23: mol_type = other DNA
ttgggagggaactcttgggaggg

SEQ ID NO: 258 - DNA, 23 bp, synthetic construct
  misc_feature 1..23: note = Synthetic: Guide Sequence for AAVS1- Like Sequence in CHO
  source 1..23: mol_type = other DNA
atcaggtgcttggaaagtagagg

SEQ ID NO: 259 - DNA, 23 bp, synthetic construct
  misc_feature 1..23: note = Synthetic: Guide Sequence for AAVS1- Like Sequence in CHO
  source 1..23: mol_type = other DNA
cggggagggaggatgctcggggg

SEQ ID NO: 260 - DNA, 23 bp, synthetic construct
  misc_feature 1..23: note = Synthetic: Guide Sequence for AAVS1- Like Sequence in CHO
  source 1..23: mol_type = other DNA
tgaggccacagcggacagcacgg

SEQ ID NO: 261 - DNA, 23 bp, synthetic construct
  misc_feature 1..23: note = Synthetic: Guide Sequence for AAVS1- Like Sequence in CHO
  source 1..23: mol_type = other DNA
tggtcacccctgtccccagaggg

SEQ ID NO: 262 - DNA, 23 bp, synthetic construct
  misc_feature 1..23: note = Synthetic: Guide Sequence for AAVS1- Like Sequence in CHO
  source 1..23: mol_type = other DNA
tccccgcctcctgccctgcaggg

SEQ ID NO: 263 - DNA, 23 bp, synthetic construct
  misc_feature 1..23: note = Synthetic: Guide Sequence for AAVS1- Like Sequence in CHO
  source 1..23: mol_type = other DNA
ggctgccctggctgtgccgcagg

SEQ ID NO: 264 - DNA, 23 bp, synthetic construct
  misc_feature 1..23: note = Synthetic: Guide Sequence for AAVS1- Like Sequence in CHO
  source 1..23: mol_type = other DNA
tccaaaagctggcattgtcaggg

SEQ ID NO: 265 - DNA, 23 bp, synthetic construct
  misc_feature 1..23: note = Synthetic: Guide Sequence for AAVS1- Like Sequence in CHO
  source 1..23: mol_type = other DNA
caatgccagcttttggatgacgg

SEQ ID NO: 266 - DNA, 23 bp, synthetic construct
  misc_feature 1..23: note = Synthetic: Guide Sequence for AAVS1- Like Sequence in CHO
  source 1..23: mol_type = other DNA
ctgggcctggctagcctcagagg

SEQ ID NO: 267 - DNA, 23 bp, synthetic construct
  misc_feature 1..23: note = Synthetic: Guide Sequence for AAVS1- Like Sequence in CHO
  source 1..23: mol_type = other DNA
ggttcactttcagtctttcatgg

SEQ ID NO: 268 - DNA, 23 bp, synthetic construct
  misc_feature 1..23: note = Synthetic: Guide Sequence for AAVS1- Like Sequence in CHO
  source 1..23: mol_type = other DNA
aggagaaacctatggggggtggg

SEQ ID NO: 269 - DNA, 23 bp, synthetic construct
  misc_feature 1..23: note = Synthetic: Guide Sequence for AAVS1- Like Sequence in CHO
  source 1..23: mol_type = other DNA
tctaaaagacagcccagcccagg

SEQ ID NO: 270 - DNA, 23 bp, synthetic construct
  misc_feature 1..23: note = Synthetic: Guide Sequence for AAVS1- Like Sequence in CHO
  source 1..23: mol_type = other DNA
cggggatcccggggaccccgggg

SEQ ID NO: 271 - DNA, 23 bp, synthetic construct
  misc_feature 1..23: note = Synthetic: Guide Sequence for AAVS1- Like Sequence in CHO
  source 1..23: mol_type = other DNA
tcttctccaggggaacaaagggg

SEQ ID NO: 272 - DNA, 23 bp, synthetic construct
  misc_feature 1..23: note = Synthetic: Guide Sequence for AAVS1- Like Sequence in CHO
  source 1..23: mol_type = other DNA
actgacacaaaaagtcagcacgg

SEQ ID NO: 273 - DNA, 23 bp, synthetic construct
  misc_feature 1..23: note = Synthetic: Guide Sequence for AAVS1- Like Sequence in CHO
  source 1..23: mol_type = other DNA
cctgaagagtcaggtcaccaagg

SEQ ID NO: 274 - DNA, 23 bp, synthetic construct
  misc_feature 1..23: note = Synthetic: Guide Sequence for AAVS1- Like Sequence in CHO
  source 1..23: mol_type = other DNA
ggaggagagtcccacctggaagg

SEQ ID NO: 275 - DNA, 23 bp, synthetic construct
  misc_feature 1..23: note = Synthetic: Guide Sequence for AAVS1- Like Sequence in CHO
  source 1..23: mol_type = other DNA
gggcagccaccagcctggaaagg

SEQ ID NO: 276 - DNA, 23 bp, synthetic construct
  misc_feature 1..23: note = Synthetic: Guide Sequence for AAVS1- Like Sequence in CHO
  source 1..23: mol_type = other DNA
agccctatttctctctcctctgg

SEQ ID NO: 277 - DNA, 23 bp, synthetic construct
  misc_feature 1..23: note = Synthetic: Guide Sequence for AAVS1- Like Sequence in CHO
  source 1..23: mol_type = other DNA
gccaccagcctggaaaggcacgg

SEQ ID NO: 278 - DNA, 23 bp, synthetic construct
  misc_feature 1..23: note = Synthetic: Guide Sequence for AAVS1- Like Sequence in CHO
  source 1..23: mol_type = other DNA
tgacaccctgagtgtggcctcgg

SEQ ID NO: 279 - DNA, 23 bp, synthetic construct
  misc_feature 1..23: note = Synthetic: Guide Sequence for AAVS1- Like Sequence in CHO
  source 1..23: mol_type = other DNA
aattaataggacatggggagggg

SEQ ID NO: 280 - DNA, 23 bp, synthetic construct
  misc_feature 1..23: note = Synthetic: Guide Sequence for AAVS1- Like Sequence in CHO
  source 1..23: mol_type = other DNA
ggctcggggggccccggggaggg

SEQ ID NO: 281 - DNA, 23 bp, synthetic construct
  misc_feature 1..23: note = Synthetic: Guide Sequence for AAVS1- Like Sequence in CHO
  source 1..23: mol_type = other DNA
taataggacatggggaggggagg

SEQ ID NO: 282 - DNA, 23 bp, synthetic construct
  misc_feature 1..23: note = Synthetic: Guide Sequence for AAVS1- Like Sequence in CHO
  source 1..23: mol_type = other DNA
ctcttctccaggggaacaaaggg

SEQ ID NO: 283 - DNA, 23 bp, synthetic construct
  misc_feature 1..23: note = Synthetic: Guide Sequence for AAVS1- Like Sequence in CHO
  source 1..23: mol_type = other DNA
tcggggcccccggggtccccggg

SEQ ID NO: 284 - DNA, 23 bp, synthetic construct
  misc_feature 1..23: note = Synthetic: Guide Sequence for AAVS1- Like Sequence in CHO
  source 1..23: mol_type = other DNA
tccctgacaatgccagcttttgg

SEQ ID NO: 285 - DNA, 23 bp, synthetic construct
  misc_feature 1..23: note = Synthetic: Guide Sequence for AAVS1- Like Sequence in CHO
  source 1..23: mol_type = other DNA
gaattaataggacatggggaggg

SEQ ID NO: 286 - DNA, 23 bp, synthetic construct
  misc_feature 1..23: note = Synthetic: Guide Sequence for AAVS1- Like Sequence in CHO
  source 1..23: mol_type = other DNA
aatgagggggtcagagacacagg

SEQ ID NO: 287 - DNA, 23 bp, synthetic construct
  misc_feature 1..23: note = Synthetic: Guide Sequence for AAVS1- Like Sequence in CHO
  source 1..23: mol_type = other DNA
gaaaacttcctttatggggccgg

SEQ ID NO: 288 - DNA, 23 bp, synthetic construct
  misc_feature 1..23: note = Synthetic: Guide Sequence for AAVS1- Like Sequence in CHO
  source 1..23: mol_type = other DNA
cttgggagggccagggactttgg

SEQ ID NO: 289 - DNA, 23 bp, synthetic construct
  misc_feature 1..23: note = Synthetic: Guide Sequence for AAVS1- Like Sequence in CHO
  source 1..23: mol_type = other DNA
cccctgcagggcaggaggcgggg

SEQ ID NO: 290 - DNA, 23 bp, synthetic construct
  misc_feature 1..23: note = Synthetic: Guide Sequence for AAVS1- Like Sequence in CHO
  source 1..23: mol_type = other DNA
tcagtgtcacttcttttgggggg

SEQ ID NO: 291 - DNA, 23 bp, synthetic construct
  misc_feature 1..23: note = Synthetic: Guide Sequence for AAVS1- Like Sequence in CHO
  source 1..23: mol_type = other DNA
atccccgttcttcttcctcctgg

SEQ ID NO: 292 - DNA, 23 bp, synthetic construct
  misc_feature 1..23: note = Synthetic: Guide Sequence for AAVS1- Like Sequence in CHO
  source 1..23: mol_type = other DNA
cttcctcctggccacagtgttgg

SEQ ID NO: 293 - DNA, 23 bp, synthetic construct
  misc_feature 1..23: note = Synthetic: Guide Sequence for AAVS1- Like Sequence in CHO
  source 1..23: mol_type = other DNA
tgcagttgggagggaactcttgg

SEQ ID NO: 294 - DNA, 23 bp, synthetic construct
  misc_feature 1..23: note = Synthetic: Guide Sequence for AAVS1- Like Sequence in CHO
  source 1..23: mol_type = other DNA
tggctcggggggccccggggagg

SEQ ID NO: 295 - DNA, 23 bp, synthetic construct
  misc_feature 1..23: note = Synthetic: Guide Sequence for AAVS1- Like Sequence in CHO
  source 1..23: mol_type = other DNA
ctgcaaaggcctgaagagtcagg

SEQ ID NO: 296 - DNA, 23 bp, synthetic construct
  misc_feature 1..23: note = Synthetic: Guide Sequence for AAVS1- Like Sequence in CHO
  source 1..23: mol_type = other DNA
ccgtgtgcggaagacgccgccgg

SEQ ID NO: 297 - DNA, 23 bp, synthetic construct
  misc_feature 1..23: note = Synthetic: Guide Sequence for AAVS1- Like Sequence in CHO
  source 1..23: mol_type = other DNA
ccccggggagggaggatgctcgg

SEQ ID NO: 298 - DNA, 23 bp, synthetic construct
  misc_feature 1..23: note = Synthetic: Guide Sequence for AAVS1- Like Sequence in CHO
  source 1..23: mol_type = other DNA
ttccaggctggtggctgccctgg

SEQ ID NO: 299 - DNA, 23 bp, synthetic construct
  misc_feature 1..23: note = Synthetic: Guide Sequence for AAVS1- Like Sequence in CHO
  source 1..23: mol_type = other DNA
aggtcaccaaggtgtctgcatgg

SEQ ID NO: 300 - DNA, 23 bp, synthetic construct
  misc_feature 1..23: note = Synthetic: Guide Sequence for AAVS1- Like Sequence in CHO
  source 1..23: mol_type = other DNA
ggcggcccctgcagggcaggagg

SEQ ID NO: 301 - DNA, 23 bp, synthetic construct
  misc_feature 1..23: note = Synthetic: Guide Sequence for AAVS1- Like Sequence in CHO
  source 1..23: mol_type = other DNA
tcggggggccccggggagggagg

SEQ ID NO: 302 - DNA, 23 bp, synthetic construct
  misc_feature 1..23: note = Synthetic: Guide Sequence for AAVS1- Like Sequence in CHO
  source 1..23: mol_type = other DNA
ccaaaagaagtgacactgaaggg

SEQ ID NO: 303 - DNA, 23 bp, synthetic construct
  misc_feature 1..23: note = Synthetic: Guide Sequence for AAVS1- Like Sequence in CHO
  source 1..23: mol_type = other DNA
ggccaggaggaagaagaacgggg

SEQ ID NO: 304 - DNA, 23 bp, synthetic construct
  misc_feature 1..23: note = Synthetic: Guide Sequence for AAVS1- Like Sequence in CHO
  source 1..23: mol_type = other DNA
gcccagggtcctggtggggtagg

SEQ ID NO: 305 - DNA, 23 bp, synthetic construct
  misc_feature 1..23: note = Synthetic: Guide Sequence for AAVS1- Like Sequence in CHO
  source 1..23: mol_type = other DNA
gctagcctcagaggagggagtgg

SEQ ID NO: 306 - DNA, 23 bp, synthetic construct
  misc_feature 1..23: note = Synthetic: Guide Sequence for AAVS1- Like Sequence in CHO
  source 1..23: mol_type = other DNA
gagggtcctttgcccctccaagg

SEQ ID NO: 307 - DNA, 23 bp, synthetic construct
  misc_feature 1..23: note = Synthetic: Guide Sequence for AAVS1- Like Sequence in CHO
  source 1..23: mol_type = other DNA
ccaccagcctggaaaggcacggg

SEQ ID NO: 308 - DNA, 23 bp, synthetic construct
  misc_feature 1..23: note = Synthetic: Guide Sequence for AAVS1- Like Sequence in CHO
  source 1..23: mol_type = other DNA
gatttctgtggtattggggttgg

SEQ ID NO: 309 - DNA, 23 bp, synthetic construct
  misc_feature 1..23: note = Synthetic: Guide Sequence for AAVS1- Like Sequence in CHO
  source 1..23: mol_type = other DNA
ctagcctcagaggagggagtggg

SEQ ID NO: 310 - DNA, 23 bp, synthetic construct
  misc_feature 1..23: note = Synthetic: Guide Sequence for AAVS1- Like Sequence in CHO
  source 1..23: mol_type = other DNA
cccggggtccccgggatccccgg

SEQ ID NO: 311 - DNA, 23 bp, synthetic construct
  misc_feature 1..23: note = Synthetic: Guide Sequence for AAVS1- Like Sequence in CHO
  source 1..23: mol_type = other DNA
catggggtggtaggtggagtggg

SEQ ID NO: 312 - DNA, 23 bp, synthetic construct
  misc_feature 1..23: note = Synthetic: Guide Sequence for AAVS1- Like Sequence in CHO
  source 1..23: mol_type = other DNA
aatgccagcttttggatgacggg

SEQ ID NO: 313 - DNA, 23 bp, synthetic construct
  misc_feature 1..23: note = Synthetic: Guide Sequence for AAVS1- Like Sequence in CHO
  source 1..23: mol_type = other DNA
gcccctgcagggcaggaggcggg

SEQ ID NO: 314 - DNA, 23 bp, synthetic construct
  misc_feature 1..23: note = Synthetic: Guide Sequence for AAVS1- Like Sequence in CHO
  source 1..23: mol_type = other DNA
gaggagaaacctatggggggtgg

SEQ ID NO: 315 - DNA, 23 bp, synthetic construct
  misc_feature 1..23: note = Synthetic: Guide Sequence for AAVS1- Like Sequence in CHO
  source 1..23: mol_type = other DNA
agaagtgacactgaagggcctgg

SEQ ID NO: 316 - DNA, 23 bp, synthetic construct
  misc_feature 1..23: note = Synthetic: Guide Sequence for AAVS1- Like Sequence in CHO
  source 1..23: mol_type = other DNA
cctccagcagatgcagaggaagg

SEQ ID NO: 317 - DNA, 23 bp, synthetic construct
  misc_feature 1..23: note = Synthetic: Guide Sequence for AAVS1- Like Sequence in CHO
  source 1..23: mol_type = other DNA
cctcttctccaggggaacaaagg

SEQ ID NO: 318 - DNA, 23 bp, synthetic construct
  misc_feature 1..23: note = Synthetic: Guide Sequence for AAVS1- Like Sequence in CHO
  source 1..23: mol_type = other DNA
ccggggtccccgggatccccggg

SEQ ID NO: 319 - DNA, 23 bp, synthetic construct
  misc_feature 1..23: note = Synthetic: Guide Sequence for AAVS1- Like Sequence in CHO
  source 1..23: mol_type = other DNA
tagcctcagaggagggagtgggg

SEQ ID NO: 320 - DNA, 23 bp, synthetic construct
  misc_feature 1..23: note = Synthetic: Guide Sequence for AAVS1- Like Sequence in CHO
  source 1..23: mol_type = other DNA
cagaggaaggggatgcagttggg

SEQ ID NO: 321 - DNA, 23 bp, synthetic construct
  misc_feature 1..23: note = Synthetic: Guide Sequence for AAVS1- Like Sequence in CHO
  source 1..23: mol_type = other DNA
ctccagcagatgcagaggaaggg

SEQ ID NO: 322 - DNA, 23 bp, synthetic construct
  misc_feature 1..23: note = Synthetic: Guide Sequence for AAVS1- Like Sequence in CHO
  source 1..23: mol_type = other DNA
gattctgtttttcctctgcctgg

SEQ ID NO: 323 - DNA, 23 bp, synthetic construct
  misc_feature 1..23: note = Synthetic: Guide Sequence for AAVS1- Like Sequence in CHO
  source 1..23: mol_type = other DNA
cttcagtgtcacttcttttgggg

SEQ ID NO: 324 - DNA, 23 bp, synthetic construct
  misc_feature 1..23: note = Synthetic: Guide Sequence for AAVS1- Like Sequence in CHO
  source 1..23: mol_type = other DNA
catagagtccctctggggacagg

SEQ ID NO: 325 - DNA, 23 bp, synthetic construct
  misc_feature 1..23: note = Synthetic: Guide Sequence for AAVS1- Like Sequence in CHO
  source 1..23: mol_type = other DNA
ggaccctcctgaggccacagcgg

SEQ ID NO: 326 - DNA, 23 bp, synthetic construct
  misc_feature 1..23: note = Synthetic: Guide Sequence for AAVS1- Like Sequence in CHO
  source 1..23: mol_type = other DNA
ccatggggtggtaggtggagtgg

SEQ ID NO: 327 - DNA, 23 bp, synthetic construct
  misc_feature 1..23: note = Synthetic: Guide Sequence for AAVS1- Like Sequence in CHO
  source 1..23: mol_type = other DNA
gacaccctgagtgtggcctcggg

SEQ ID NO: 328 - DNA, 23 bp, synthetic construct
  misc_feature 1..23: note = Synthetic: Guide Sequence for AAVS1- Like Sequence in CHO
  source 1..23: mol_type = other DNA
atgcttagaagtttccagaaagg

SEQ ID NO: 329 - DNA, 23 bp, synthetic construct
  misc_feature 1..23: note = Synthetic: Guide Sequence for AAVS1- Like Sequence in CHO
  source 1..23: mol_type = other DNA
agctgggccctggaagcctgcgg

SEQ ID NO: 330 - DNA, 23 bp, synthetic construct
  misc_feature 1..23: note = Synthetic: Guide Sequence for AAVS1- Like Sequence in CHO
  source 1..23: mol_type = other DNA
taccacagaaatcaggtgcttgg

SEQ ID NO: 331 - DNA, 23 bp, synthetic construct
  misc_feature 1..23: note = Synthetic: Guide Sequence for AAVS1- Like Sequence in CHO
  source 1..23: mol_type = other DNA
accccaataccacagaaatcagg

SEQ ID NO: 332 - DNA, 23 bp, synthetic construct
  misc_feature 1..23: note = Synthetic: Guide Sequence for AAVS1- Like Sequence in CHO
  source 1..23: mol_type = other DNA
ttctacaggtaaaaaaactaagg

SEQ ID NO: 333 - DNA, 23 bp, synthetic construct
  misc_feature 1..23: note = Synthetic: Guide Sequence for AAVS1- Like Sequence in CHO
  source 1..23: mol_type = other DNA
ggcccctgcagggcaggaggcgg

SEQ ID NO: 334 - DNA, 23 bp, synthetic construct
  misc_feature 1..23: note = Synthetic: Guide Sequence for AAVS1- Like Sequence in CHO
  source 1..23: mol_type = other DNA
ctccccgcctcctgccctgcagg

SEQ ID NO: 335 - DNA, 23 bp, synthetic construct
  misc_feature 1..23: note = Synthetic: Guide Sequence for AAVS1- Like Sequence in CHO
  source 1..23: mol_type = other DNA
tctctgaccccctcatttgtggg

SEQ ID NO: 336 - DNA, 23 bp, synthetic construct
  misc_feature 1..23: note = Synthetic: Guide Sequence for AAVS1- Like Sequence in CHO
  source 1..23: mol_type = other DNA
ggagaaacctatggggggtgggg

SEQ ID NO: 337 - DNA, 23 bp, synthetic construct
  misc_feature 1..23: note = Synthetic: Guide Sequence for AAVS1- Like Sequence in CHO
  source 1..23: mol_type = other DNA
acagcccagcccaggacagacgg

SEQ ID NO: 338 - DNA, 23 bp, synthetic construct
  misc_feature 1..23: note = Synthetic: Guide Sequence for AAVS1- Like Sequence in CHO
  source 1..23: mol_type = other DNA
cctgtatttgaggttggcctggg

SEQ ID NO: 339 - DNA, 23 bp, synthetic construct
  misc_feature 1..23: note = Synthetic: Guide Sequence for AAVS1- Like Sequence in CHO
  source 1..23: mol_type = other DNA
agccagggcagccaccagcctgg

SEQ ID NO: 340 - DNA, 23 bp, synthetic construct
  misc_feature 1..23: note = Synthetic: Guide Sequence for AAVS1- Like Sequence in CHO
  source 1..23: mol_type = other DNA
agcctcagaggagggagtggggg

SEQ ID NO: 341 - DNA, 23 bp, synthetic construct
  misc_feature 1..23: note = Synthetic: Guide Sequence for AAVS1- Like Sequence in CHO
  source 1..23: mol_type = other DNA
gttcagtgtttcactcatccagg

SEQ ID NO: 342 - DNA, 23 bp, synthetic construct
  misc_feature 1..23: note = Synthetic: Guide Sequence for AAVS1- Like Sequence in CHO
  source 1..23: mol_type = other DNA
ctgactcttcaggcctttgcagg

SEQ ID NO: 343 - DNA, 23 bp, synthetic construct
  misc_feature 1..23: note = Synthetic: Guide Sequence for AAVS1- Like Sequence in CHO
  source 1..23: mol_type = other DNA
atcccccactccctcctctgagg

SEQ ID NO: 344 - DNA, 23 bp, synthetic construct
  misc_feature 1..23: note = Synthetic: Guide Sequence for AAVS1- Like Sequence in CHO
  source 1..23: mol_type = other DNA
cccaaaagaagtgacactgaagg

SEQ ID NO: 345 - DNA, 23 bp, synthetic construct
  misc_feature 1..23: note = Synthetic: Guide Sequence for AAVS1- Like Sequence in CHO
  source 1..23: mol_type = other DNA
tggccaggaggaagaagaacggg

SEQ ID NO: 346 - DNA, 23 bp, synthetic construct
  misc_feature 1..23: note = Synthetic: Guide Sequence for AAVS1- Like Sequence in CHO
  source 1..23: mol_type = other DNA
gggaggaaggttatgggatgggg

SEQ ID NO: 347 - DNA, 23 bp, synthetic construct
  misc_feature 1..23: note = Synthetic: Guide Sequence for AAVS1- Like Sequence in CHO
  source 1..23: mol_type = other DNA
cctgaggcttcctgcactctagg

SEQ ID NO: 348 - DNA, 23 bp, synthetic construct
  misc_feature 1..23: note = Synthetic: Guide Sequence for AAVS1- Like Sequence in CHO
  source 1..23: mol_type = other DNA
tagtttttttacctgtagaatgg

SEQ ID NO: 349 - DNA, 23 bp, synthetic construct
  misc_feature 1..23: note = Synthetic: Guide Sequence for AAVS1- Like Sequence in CHO
  source 1..23: mol_type = other DNA
aagtggggtagggaacaaggtgg

SEQ ID NO: 350 - DNA, 23 bp, synthetic construct
  misc_feature 1..23: note = Synthetic: Guide Sequence for AAVS1- Like Sequence in CHO
  source 1..23: mol_type = other DNA
gggtcacaggaagtggggtaggg

SEQ ID NO: 351 - DNA, 23 bp, synthetic construct
  misc_feature 1..23: note = Synthetic: Guide Sequence for AAVS1- Like Sequence in CHO
  source 1..23: mol_type = other DNA
cacctgatttctgtggtattggg

SEQ ID NO: 352 - DNA, 23 bp, synthetic construct
  misc_feature 1..23: note = Synthetic: Guide Sequence for AAVS1- Like Sequence in CHO
  source 1..23: mol_type = other DNA
tttgcaactgggtctcatgtggg

SEQ ID NO: 353 - DNA, 23 bp, synthetic construct
  misc_feature 1..23: note = Synthetic: Guide Sequence for AAVS1- Like Sequence in CHO
  source 1..23: mol_type = other DNA
gagaaacctatggggggtggggg

SEQ ID NO: 354 - DNA, 23 bp, synthetic construct
  misc_feature 1..23: note = Synthetic: Guide Sequence for AAVS1- Like Sequence in CHO
  source 1..23: mol_type = other DNA
gagggaggaggggtggctcgggg

SEQ ID NO: 355 - DNA, 23 bp, synthetic construct
  misc_feature 1..23: note = Synthetic: Guide Sequence for AAVS1- Like Sequence in CHO
  source 1..23: mol_type = other DNA
gcctgtaatcccacaaatgaggg

SEQ ID NO: 356 - DNA, 23 bp, synthetic construct
  misc_feature 1..23: note = Synthetic: Guide Sequence for AAVS1- Like Sequence in CHO
  source 1..23: mol_type = other DNA
gcagaggaaggggatgcagttgg

SEQ ID NO: 357 - DNA, 23 bp, synthetic construct
  misc_feature 1..23: note = Synthetic: Guide Sequence for AAVS1- Like Sequence in CHO
  source 1..23: mol_type = other DNA
acctgatttctgtggtattgggg

SEQ ID NO: 358 - DNA, 23 bp, synthetic construct
  misc_feature 1..23: note = Synthetic: Guide Sequence for AAVS1- Like Sequence in CHO
  source 1..23: mol_type = other DNA
aaaccagaggagagagaaatagg

SEQ ID NO: 359 - DNA, 23 bp, synthetic construct
  misc_feature 1..23: note = Synthetic: Guide Sequence for AAVS1- Like Sequence in CHO
  source 1..23: mol_type = other DNA
aaccagaggagagagaaataggg

SEQ ID NO: 360 - DNA, 23 bp, synthetic construct
  misc_feature 1..23: note = Synthetic: Guide Sequence for AAVS1- Like Sequence in CHO
  source 1..23: mol_type = other DNA
gaggagagagaaatagggcttgg

SEQ ID NO: 361 - DNA, 23 bp, synthetic construct
  misc_feature 1..23: note = Synthetic: Guide Sequence for AAVS1- Like Sequence in CHO
  source 1..23: mol_type = other DNA
ctgcagggcaggaggcggggagg

SEQ ID NO: 362 - DNA, 23 bp, synthetic construct
  misc_feature 1..23: note = Synthetic: Guide Sequence for AAVS1- Like Sequence in CHO
  source 1..23: mol_type = other DNA
aggaaggggatgcagttgggagg

SEQ ID NO: 363 - DNA, 23 bp, synthetic construct
  misc_feature 1..23: note = Synthetic: Guide Sequence for AAVS1- Like Sequence in CHO
  source 1..23: mol_type = other DNA
ggtattggggttggaacctgagg

SEQ ID NO: 364 - DNA, 23 bp, synthetic construct
  misc_feature 1..23: note = Synthetic: Guide Sequence for AAVS1- Like Sequence in CHO
  source 1..23: mol_type = other DNA
ttttgcaactgggtctcatgtgg

SEQ ID NO: 365 - DNA, 23 bp, synthetic construct
  misc_feature 1..23: note = Synthetic: Guide Sequence for AAVS1- Like Sequence in CHO
  source 1..23: mol_type = other DNA
gggaggaggggtggctcgggggg

SEQ ID NO: 366 - DNA, 23 bp, synthetic construct
  misc_feature 1..23: note = Synthetic: Guide Sequence for AAVS1- Like Sequence in CHO
  source 1..23: mol_type = other DNA
tccccttcctctgcatctgctgg

SEQ ID NO: 367 - DNA, 23 bp, synthetic construct
  misc_feature 1..23: note = Synthetic: Guide Sequence for AAVS1- Like Sequence in CHO
  source 1..23: mol_type = other DNA
aggaagtggggtagggaacaagg

SEQ ID NO: 368 - DNA, 23 bp, synthetic construct
  misc_feature 1..23: note = Synthetic: Guide Sequence for AAVS1- Like Sequence in CHO
  source 1..23: mol_type = other DNA
aggggaggaaggttatgggatgg

SEQ ID NO: 369 - DNA, 23 bp, synthetic construct
  misc_feature 1..23: note = Synthetic: Guide Sequence for AAVS1- Like Sequence in CHO
  source 1..23: mol_type = other DNA
cctgtaatcccacaaatgagggg

SEQ ID NO: 370 - DNA, 23 bp, synthetic construct
  misc_feature 1..23: note = Synthetic: Guide Sequence for AAVS1- Like Sequence in CHO
  source 1..23: mol_type = other DNA
agtttttttacctgtagaatggg

SEQ ID NO: 371 - DNA, 23 bp, synthetic construct
  misc_feature 1..23: note = Synthetic: Guide Sequence for AAVS1- Like Sequence in CHO
  source 1..23: mol_type = other DNA
aggaagaagaacggggatggggg

SEQ ID NO: 372 - DNA, 23 bp, synthetic construct
  misc_feature 1..23: note = Synthetic: Guide Sequence for AAVS1- Like Sequence in CHO
  source 1..23: mol_type = other DNA
tgcagggcaggaggcggggaggg

SEQ ID NO: 373 - DNA, 23 bp, synthetic construct
  misc_feature 1..23: note = Synthetic: Guide Sequence for AAVS1- Like Sequence in CHO
  source 1..23: mol_type = other DNA
ggaaggggatgcagttgggaggg

SEQ ID NO: 374 - DNA, 23 bp, synthetic construct
  misc_feature 1..23: note = Synthetic: Guide Sequence for AAVS1- Like Sequence in CHO
  source 1..23: mol_type = other DNA
caaagtcactgtgtagatgaagg

SEQ ID NO: 375 - DNA, 23 bp, synthetic construct
  misc_feature 1..23: note = Synthetic: Guide Sequence for AAVS1- Like Sequence in CHO
  source 1..23: mol_type = other DNA
gtggccaggaggaagaagaacgg

SEQ ID NO: 376 - DNA, 23 bp, synthetic construct
  misc_feature 1..23: note = Synthetic: Guide Sequence for AAVS1- Like Sequence in CHO
  source 1..23: mol_type = other DNA
tccagcagatgcagaggaagggg

SEQ ID NO: 377 - DNA, 23 bp, synthetic construct
  misc_feature 1..23: note = Synthetic: Guide Sequence for AAVS1- Like Sequence in CHO
  source 1..23: mol_type = other DNA
agggaggaggggtggctcggggg

SEQ ID NO: 378 - DNA, 23 bp, synthetic construct
  misc_feature 1..23: note = Synthetic: Guide Sequence for AAVS1- Like Sequence in CHO
  source 1..23: mol_type = other DNA
aacctatggggggtgggggtggg

SEQ ID NO: 379 - DNA, 23 bp, synthetic construct
  misc_feature 1..23: note = Synthetic: Guide Sequence for AAVS1- Like Sequence in CHO
  source 1..23: mol_type = other DNA
ggggaggggaggaaggttatggg

SEQ ID NO: 380 - DNA, 23 bp, synthetic construct
  misc_feature 1..23: note = Synthetic: Guide Sequence for AAVS1- Like Sequence in CHO
  source 1..23: mol_type = other DNA
cacccacccccaccccccatagg

SEQ ID NO: 381 - DNA, 23 bp, synthetic construct
  misc_feature 1..23: note = Synthetic: Guide Sequence for AAVS1- Like Sequence in CHO
  source 1..23: mol_type = other DNA
aaacctatggggggtgggggtgg

SEQ ID NO: 382 - DNA, 23 bp, synthetic construct
  misc_feature 1..23: note = Synthetic: Guide Sequence for AAVS1- Like Sequence in CHO
  source 1..23: mol_type = other DNA
ggaggaagaagaacggggatggg

SEQ ID NO: 383 - DNA, 23 bp, synthetic construct
  misc_feature 1..23: note = Synthetic: Guide Sequence for AAVS1- Like Sequence in CHO
  source 1..23: mol_type = other DNA
gaggaagaagaacggggatgggg

SEQ ID NO: 384 - DNA, 23 bp, synthetic construct
  misc_feature 1..23: note = Synthetic: Guide Sequence for AAVS1- Like Sequence in CHO
  source 1..23: mol_type = other DNA
ggagggaggaggggtggctcggg

SEQ ID NO: 385 - DNA, 23 bp, synthetic construct
  misc_feature 1..23: note = Synthetic: Guide Sequence for AAVS1- Like Sequence in CHO
  source 1..23: mol_type = other DNA
ggttggcctgggctacacagggg

SEQ ID NO: 386 - DNA, 23 bp, synthetic construct
  misc_feature 1..23: note = Synthetic: Guide Sequence for AAVS1- Like Sequence in CHO
  source 1..23: mol_type = other DNA
agaggagggagtgggggattggg

SEQ ID NO: 387 - DNA, 23 bp, synthetic construct
  misc_feature 1..23: note = Synthetic: Guide Sequence for AAVS1- Like Sequence in CHO
  source 1..23: mol_type = other DNA
gaggttggcctgggctacacagg

SEQ ID NO: 388 - DNA, 23 bp, synthetic construct
  misc_feature 1..23: note = Synthetic: Guide Sequence for AAVS1- Like Sequence in CHO
  source 1..23: mol_type = other DNA
aggttggcctgggctacacaggg

SEQ ID NO: 389 - DNA, 23 bp, synthetic construct
  misc_feature 1..23: note = Synthetic: Guide Sequence for AAVS1- Like Sequence in CHO
  source 1..23: mol_type = other DNA
tggggaggggaggaaggttatgg

SEQ ID NO: 390 - DNA, 23 bp, synthetic construct
  misc_feature 1..23: note = Synthetic: Guide Sequence for AAVS1- Like Sequence in CHO
  source 1..23: mol_type = other DNA
gaaagtagaggcaggagggttgg

SEQ ID NO: 391 - DNA, 23 bp, synthetic construct
  misc_feature 1..23: note = Synthetic: Guide Sequence for AAVS1- Like Sequence in CHO
  source 1..23: mol_type = other DNA
gaggagggagtgggggattgggg

SEQ ID NO: 392 - DNA, 23 bp, synthetic construct
  misc_feature 1..23: note = Synthetic: Guide Sequence for AAVS1- Like Sequence in CHO
  source 1..23: mol_type = other DNA
ggggaggaaggttatgggatggg

SEQ ID NO: 393 - DNA, 23 bp, synthetic construct
  misc_feature 1..23: note = Synthetic: Guide Sequence for AAVS1- Like Sequence in CHO
  source 1..23: mol_type = other DNA
agagtgcttgcctagagtgcagg

SEQ ID NO: 394 - DNA, 23 bp, synthetic construct
  misc_feature 1..23: note = Synthetic: Guide Sequence for AAVS1- Like Sequence in CHO
  source 1..23: mol_type = other DNA
aggagggagtgggggattggggg

SEQ ID NO: 395 - DNA, 23 bp, synthetic construct
  misc_feature 1..23: note = Synthetic: Guide Sequence for AAVS1- Like Sequence in CHO
  source 1..23: mol_type = other DNA
cagaggagggagtgggggattgg

SEQ ID NO: 396 - DNA, 23 bp, synthetic construct
  misc_feature 1..23: note = Synthetic: Guide Sequence for AAVS1- Like Sequence in CHO
  source 1..23: mol_type = other DNA
aggaggaagaagaacggggatgg

SEQ ID NO: 397 - DNA, 23 bp, synthetic construct
  misc_feature 1..23: note = Synthetic: Guide Sequence for AAVS1- Like Sequence in CHO
  source 1..23: mol_type = other DNA
ttttttcccctgtgtagcccagg

SEQ ID NO: 398 - DNA, 23 bp, synthetic construct
  misc_feature 1..23: note = Synthetic: Guide Sequence for AAVS1- Like Sequence in CHO
  source 1..23: mol_type = other DNA
ggtgcttggaaagtagaggcagg

SEQ ID NO: 399 - DNA, 23 bp, synthetic construct
  misc_feature 1..23: note = Synthetic: Guide Sequence for AAVS1- Like Sequence in CHO
  source 1..23: mol_type = other DNA
cttggaaagtagaggcaggaggg

SEQ ID NO: 400 - DNA, 23 bp, synthetic construct
  misc_feature 1..23: note = Synthetic: Guide Sequence for AAVS1- Like Sequence in CHO
  source 1..23: mol_type = other DNA
ctgtaatcccacaaatgaggggg

SEQ ID NO: 401 - DNA, 23 bp, synthetic construct
  misc_feature 1..23: note = Synthetic: Guide Sequence for AAVS1- Like Sequence in CHO
  source 1..23: mol_type = other DNA
aggacatggggaggggaggaagg

SEQ ID NO: 402 - DNA, 23 bp, synthetic construct
  misc_feature 1..23: note = Synthetic: Guide Sequence for AAVS1- Like Sequence in CHO
  source 1..23: mol_type = other DNA
tcatctacacagtgactttgagg

SEQ ID NO: 403 - DNA, 23 bp, synthetic construct
  misc_feature 1..23: note = Synthetic: Guide Sequence for AAVS1- Like Sequence in CHO
  source 1..23: mol_type = other DNA
gagggagtgggggattggggggg

SEQ ID NO: 404 - DNA, 23 bp, synthetic construct
  misc_feature 1..23: note = Synthetic: Guide Sequence for AAVS1- Like Sequence in CHO
  source 1..23: mol_type = other DNA
gggagggaggaggggtggctcgg

SEQ ID NO: 405 - DNA, 23 bp, synthetic construct
  misc_feature 1..23: note = Synthetic: Guide Sequence for AAVS1- Like Sequence in CHO
  source 1..23: mol_type = other DNA
agggagtgggggattgggggggg

SEQ ID NO: 406 - DNA, 23 bp, synthetic construct
  misc_feature 1..23: note = Synthetic: Guide Sequence for AAVS1- Like Sequence in CHO
  source 1..23: mol_type = other DNA
aacaacaaaaacaaaaccagagg

SEQ ID NO: 407 - DNA, 23 bp, synthetic construct
  misc_feature 1..23: note = Synthetic: Guide Sequence for AAVS1- Like Sequence in CHO
  source 1..23: mol_type = other DNA
ctatggggggtgggggtgggtgg

SEQ ID NO: 408 - DNA, 23 bp, synthetic construct
  misc_feature 1..23: note = Synthetic: Guide Sequence for AAVS1- Like Sequence in CHO
  source 1..23: mol_type = other DNA
ggagggagtgggggattgggggg

SEQ ID NO: 409 - DNA, 23 bp, synthetic construct
  misc_feature 1..23: note = Synthetic: Guide Sequence for AAVS1- Like Sequence in CHO
  source 1..23: mol_type = other DNA
tgcctgtaatcccacaaatgagg

SEQ ID NO: 410 - DNA, 23 bp, synthetic construct
  misc_feature 1..23: note = Synthetic: Guide Sequence for AAVS1- Like Sequence in CHO
  source 1..23: mol_type = other DNA
gagtgggggattggggggggggg

SEQ ID NO: 411 - DNA, 23 bp, synthetic construct
  misc_feature 1..23: note = Synthetic: Guide Sequence for AAVS1- Like Sequence in CHO
  source 1..23: mol_type = other DNA
agggcaggaggcggggagggagg

SEQ ID NO: 412 - DNA, 23 bp, synthetic construct
  misc_feature 1..23: note = Synthetic: Guide Sequence for AAVS1- Like Sequence in CHO
  source 1..23: mol_type = other DNA
caggaggcggggagggaggaggg

SEQ ID NO: 413 - DNA, 23 bp, synthetic construct
  misc_feature 1..23: note = Synthetic: Guide Sequence for AAVS1- Like Sequence in CHO
  source 1..23: mol_type = other DNA
aggaggcggggagggaggagggg

SEQ ID NO: 414 - DNA, 23 bp, synthetic construct
  misc_feature 1..23: note = Synthetic: Guide Sequence for AAVS1- Like Sequence in CHO
  source 1..23: mol_type = other DNA
ggagtgggggattgggggggggg

SEQ ID NO: 415 - DNA, 23 bp, synthetic construct
  misc_feature 1..23: note = Synthetic: Guide Sequence for AAVS1- Like Sequence in CHO
  source 1..23: mol_type = other DNA
gggagtgggggattggggggggg

SEQ ID NO: 416 - DNA, 23 bp, synthetic construct
  misc_feature 1..23: note = Synthetic: Guide Sequence for AAVS1- Like Sequence in CHO
  source 1..23: mol_type = other DNA
gcaggaggcggggagggaggagg

SEQ ID NO: 417 - DNA, 23 bp, synthetic construct
  misc_feature 1..23: note = Synthetic: Guide Sequence for AAVS1- Like Sequence in CHO
  source 1..23: mol_type = other DNA
aggcggggagggaggaggggtgg

SEQ ID NO: 418 - DNA, 23 bp, synthetic construct
  misc_feature 1..23: note = Synthetic: Guide Sequence for AAVS1- Like Sequence in CHO
  source 1..23: mol_type = other DNA
gcttggaaagtagaggcaggagg

SEQ ID NO: 419 - DNA, 23 bp, synthetic construct
  misc_feature 1..23: note = Synthetic: Guide Sequence for AAVS1- Like Sequence in CHO
  source 1..23: mol_type = other DNA
gagagagagagagagagttgagg

(19) United States
(12) Patent Application Publication
(10) Pub. No.: US 2026/0265784 A1
(43) Pub. Date: Sep. 10, 2026

(54) MAMMALIAN CELLS COMPRISING INTEGRATED CAS9 GENES TO PRODUCE STABLE INTEGRATION SITES, AND MAMMALIAN CELLS COMPRISING STABLE INTEGRATION SITES AND OTHER SITES

(71) Applicant: Regeneron Pharmaceuticals, Inc, Tarrytown, NY (US)

(72) Inventors: Michael Goren, Tarrytown, NY (US); Darya Burakov, Tarrytown, NY (US); Gang Chen, Yorktown Heights, NY (US); Yu Zhao, Williston Park, NY (US); Dipali Deshpande, Tarrytown, NY (US)

(21) Appl. No.: 19/554,177

(22) Filed: Mar. 2, 2026

Related U.S. Application Data
(62) Division of application No. 18/047,357, filed on Oct. 18, 2022.
(60) Provisional application No. 63/256,675, filed on Oct. 18, 2021.

(51) Int. Cl. C12N 15/90; C12N 5/00; C12N 9/22; C12N 15/11

(52) U.S. Cl. CPC C12N 15/907; C12N 5/00; C12N 9/22; C12N 15/11; C12N 2310/20; C12N 2800/80; C12N 2820/002

(57) ABSTRACT

The present inventions provide mammalian cells that comprise multiple Stable Integration Sites. The inventions provide sites introduced genomically into a Genomic Safe Harbor and introduced genomically outside of that particular Genomic Safe Harbor, including but not limited to another Genomic Safe Harbor. Polynucleotides of interest that encode polypeptides or RNAs of interest can be inserted into the Stable Integration Sites provided according to the inventions. The cells and methods of the inventions can be used for the high yield production of any protein, including viral proteins. Additionally, the cells and methods of the inventions are useful for production of viral vectors, such as AAV, antibodies and other proteins.

This application claims priority to U.S. Application Ser. No. 63/256,675, filed Oct. 18, 2021, which is hereby incorporated by reference in its entirety.

### FIELD OF THE INVENTIONS

The present inventions provide mammalian cells (including cell lines), including human and rodent cells (including cell lines), that comprise multiple Stable Integration Sites (SIS), which can be produced using integrated Cas9 genes. The inventions provide Stable Integration Sites (1) introduced genomically into Genomic Safe Harbors (GSH), for example AAVS1 (Adeno-Associated Virus Integration Site 1) and AAVS1-like, and (2) introduced genomically outside of that particular Genomic Safe Harbor, such as a different Genomic Safe Harbor or other region that is not a Genomic Safe Harbor. Polydeoxyribonucleotides of interest that encode polypeptides or RNAs of interest can be inserted into the Stable Integration Sites provided according to the inventions.

### REFERENCE TO ELECTRONIC SEQUENCE LISTING

The application contains a Sequence Listing which has been submitted electronically in .XML format and is hereby incorporated by reference in its entirety. Said .XML copy, created on Oct. 7, 2022, is named “135975-97402.xml” and is 709,205 bytes in size. The sequence listing contained in this. XML file is part of the specification and is hereby incorporated by reference herein in its entirety.

### BACKGROUND OF THE INVENTIONS

Mammalian cell lines are the preferred approach for producing commercial quantities of therapeutic proteins, such as antibodies. However, it has been reported that modified mammalian cells often exhibit production decreases due to genetic and epigenetic instability. Hilliard and Lee, Biotech. Bioeng. 118:659-75 (2021).

Integration of polynucleotides is the preferred approach for creating and maintaining transformed cells. Integration of particular sequences into human AAVS1 is discussed in Liu et al., BMC Research Note, 7:626 (2014) and Ramachandra et al., Nucl. Acids Res. 39: e107 (2011). Human AAVS1 is known as a Genomic Safe Harbor. Papapetrou et al., Molecular Therapy 24:678-84 (2016). Gaidukov et al., Nucl. Acids Res. 46:4072-86 (2018) have disclosed sites for DNA integration into landing pads.

Chinese hamster ovary (CHO) cells and baby hamster kidney cells (BHK) are used in the production of therapeutic proteins, and hamster genomes have been extensively studied. Hamaker and Lee have reported on CHO chromosomal loci as potential sites for stable integration and refers to them as “genomic hot spots”. Curr. Op. Chem. Eng. 22:152-60 (2018) at 153. At Table 1, Hamaker and Lee identify 30 hot spot loci, of which 17 are identified by gene and 13 are unannotated. Curr. Op. Chem. Eng. 22:152-60 (2018) at 154. This work was followed by Hilliard and Lee, who sought to identify safe harbor regions in CHO using an epigenome analysis. Hilliard and Lee, Biotech. Bioeng. 118:659-75 (2021). The authors determined that 10.9% of the CHO genome contained chromatin structures with enhanced genetic and epigenetic stability. The authors further determined that of the 30 hot spots identified Table 1 by Hamaker and Lee, five of which overlapped with stable regions determined by high throughput chromosome conformation capture (Hi-C). The closest genes to the regions were ALDH5A1, SMAD6 and CLCN3, and two other regions were unannotated. Hilliard and Lee, Biotech. Bioeng. 118: 659-75 (2021) at Supplementary Table 3 (S3). Gaidukov et al., Nucl. Acids Res. 46: 4072-86 (2018) at Table 1 also identifies loci for integration in CHO cells. Lee et al., Scientific Reps. 5:8572 (2015) identifies the COSMC locus.

The present inventions advantageously employ an integrated Cas9 gene to efficiently create mammalian cell intermediates that are further modified to provide mammalian cells having multiple Stable Integration Sites for stable integration of multiple DNA cassettes and other polydeoxyribonucleotides of interest. According to the inventions, a Stable Integration Site can be located in a Genomic Safe Harbor or other regions, including newly-identified Genomic Safe Harbors.

### SUMMARY OF THE INVENTIONS

The inventions provide mammalian cells, wherein any cell thereof can comprise a first Stable Integration Site located in a Genomic Safe Harbor and a second Stable Integration Site that is not located in the Genomic Safe Harbor, wherein the first Stable Integration Site comprises a first reporter gene encoding a first reporter protein and the second Stable Integration Site comprises a second reporter gene encoding a second reporter protein, wherein the first reporter protein and the second reporter protein are different. The first and second Stable Integration Sites can comprise recombinase recognition sites (RRSs). The first and second reporter genes can be under the control of SV40 promoters. The first and second reporter genes can be fluorescent proteins. The cells can further comprise a polynucleotide encoding a repressor protein under the control of a CMV promoter. The cells can be a Human Amniotic Epithelial, HEK 293, CHO or a BHK Cell. The polynucleotide encoding a protein of interest can be inserted into the first Stable Integration Site or the second Stable Integration Site. The second Stable Integration Site can be located in a second Genomic Safe Harbor that is different from the first Genomic Safe Harbor or in a region that is not a Genomic Safe Harbor.

The inventions also provide mammalian cells, wherein any cell thereof can comprise a first Stable Integration Site located in a Genomic Safe Harbor and a second Stable Integration Site that is not located in the first Genomic Safe Harbor, wherein the first Stable Integration Site comprises first polynucleotide encoding a first protein and the second Stable Integration Site comprises a second polynucleotide encoding a second protein. The first and second proteins can be viral proteins, such as an adenovirus associated virus protein or an adenovirus protein. For example, mammalian cells can comprise a polynucleotide encoding an adeno-associated virus protein and a polynucleotide encoding an adenovirus protein. Other polynucleotides encoding proteins include, but are not limited to, antibody genes, for example. Cells can have the second Stable Integration Site located in a second Genomic Safe Harbor that is different from the first Genomic Safe Harbor that the first Stable Integration Site is located in, or in a region that is not a Genomic Safe Harbor.

The inventions further provide a mammalian cells, wherein any cell thereof can comprise a first Stable Integration Site located in a Genomic Safe Harbor and a second Stable Integration Site that is not located in the Genomic Safe Harbor, wherein the first Stable Integration Site comprises a polynucleotide encoding a first reporter gene encoding a first reporter protein and the second Stable Integration Site comprises a polynucleotide encoding Cas9 and a polynucleotide encoding a second reporter gene encoding a second reporter protein, wherein the first reporter protein and the second reporter protein are different. The second Stable Integration Site can further comprise a selection marker gene and an internal ribosome entry site (IRES). The first and second Stable Integration Sites can comprise recombinase recognition sites. The first and second reporter genes can be under the control of SV40 promoters. The first and second reporter genes can be fluorescent proteins. The cell can further comprise a polynucleotide encoding a repressor (for example TetR) under the control of a promoter (for example, CMV). The cell can be a Human Amniotic Epithelial Cell, HEK293, CHO or a BHK Cell. The polynucleotide encoding a protein of interest can be inserted into the first Stable Integration Site or the second Stable Integration Site. The selection marker protein can confer drug resistance. The second reporter gene, the selection marker gene, the IRES and an SV40 promoter can be arranged on a DNA cassette. The cell can further comprise a polynucleotide encoding a repressor protein under the control of a promoter (for example, CMV). The second Stable Integration Site can be located in a second Genomic Safe Harbor that is different from the first Genomic Safe Harbor that the first Stable Integration Site is located in or in a region that is not a Genomic Safe Harbor. The first reporter gene can be flanked by a 5′ genomic safe harbor homology arm and a 3′ genomic safe harbor homology arm. The 5′ genomic safe harbor homology arm can comprise a CRISPR sgRNA target site and the 3′ genomic safe harbor homology arm can comprise a CRISPR sgRNA target site.

The inventions further provide methods for making at least one protein of interest, wherein any method thereof can comprise: (a) providing mammalian cells comprising a first Stable Integration Site located in a Genomic Safe Harbor and a second Stable Integration Site that is not located in the first Genomic Safe Harbor, wherein the first Stable Integration Site comprises a first reporter gene encoding a first reporter protein and the second Stable Integration Site comprises a second reporter gene encoding a second reporter protein, wherein the first reporter protein and the second reporter protein are different, and wherein the first and second Stable Integration Sites comprise recombinase recognition sites; (b) introducing a polynucleotide encoding the protein of interest into a Stable Integration Site by recombinase mediated cassette exchange, and (c) culturing the mammalian cell of under conditions that allow expression of the polynucleotide encoding the polynucleotide of interest. The first and second reporter genes can be under the control of SV40 promoters. The first and second reporter genes can be fluorescent proteins. The cell can further comprise a polynucleotide encoding a repressor protein under the control of a CMV promoter. The cell can be a Human Amniotic Epithelial, HEK 293, CHO or a BHK Cell. The polynucleotide encoding a protein of interest can be inserted into the first Stable Integration Site or the second Stable Integration Site. The second Stable Integration Site can be located in a second Genomic Safe Harbor that is different from the first Genomic Safe Harbor that first Stable Integration Site is located in, or in a region that is not a Genomic Safe Harbor. The first Stable Integration Site comprises a first polynucleotide encoding a first protein and the second Stable Integration Site comprises a second polynucleotide encoding a second protein. The first and second proteins can be viral proteins, such as an adenovirus associated virus protein or an adenovirus protein. For example, the mammalian cell can comprise a polynucleotide encoding an adeno-associated virus protein and a polynucleotide encoding an adenovirus protein. Other polynucleotides encoding proteins include, but are not limited to, antibody genes, for example. The second Stable Integration Site also can be located in a region that is not a Genomic Safe Harbor.

The inventions further provide methods of creating mammalian cells with multiple Stable Integration Sites, wherein any method thereof can comprise: (A) providing a mammalian cell comprising a first DNA cassette comprising in 5′ to 3′ order a polynucleotide encoding the first lox site, a promoter, a selection marker gene encoding a selection marker protein, an IRES, a first reporter gene encoding a first reporter protein, a promoter operably linked to an operator, a Cas9 gene and the second lox site; (B) integrating a second DNA cassette comprising in a 5′ to 3′ order a polynucleotide comprising a first Genomic Safe Harbor homology arm containing a CRISPR sgRNA target site, a third lox site, a second reporter gene encoding a second reporter protein, a forth lox site and a second Genomic Safe Harbor homology arm containing an CRISPR sgRNA target site, wherein the first lox site, the second lox site, the third lox site and the forth lox site are different, wherein the first and second guide arms can contain a region with alterations (if needed to avoid recreating a targetable site), and wherein the second reporter protein is different from the first reporter protein; (C) exchanging the first DNA cassette with a third DNA cassette, wherein the third DNA cassette comprises in a 5′ to 3′ order a polynucleotide encoding the first lox site, a promoter, a third reporter gene encoding a third reporter protein, and the second lox site, wherein the third reporter protein is different from the second reporter protein, thereby providing the mammalian cell with multiple Stable Integration Sites. The mammalian cells can be Human Amniotic Epithelial Cells, HEK 293 Cells, CHO Cells or BHK Cells. Reporter genes for use can fluorescent proteins. The cell of step (A) can further comprise a polynucleotide encoding a repressor (for example, TetR) under the control of a promoter (for example, CMV). The cell of step (B) can further comprise a polynucleotide encoding a repressor (for example, TetR) under the control of a promoter (for example, CMV). The cell of step (C) can further comprise a polynucleotide encoding a repressor (for example, TetR) under the control of a promoter (for example, CMV). The selection marker protein can confer drug resistance. Lox sites are the most commonly used type of RRS; however, different RRSs can be used as well.

The inventions also provide methods of creating a mammalian cell with multiple recombinase-mediated cassette exchange sites, wherein any method thereof can comprise: (A) randomly integrating a promoter and polynucleotide encoding a repressor into the cell genome, wherein the repressor can bind to a ligand; (B) randomly integrating into the cell genome a first DNA cassette comprising in 5′ to 3′ order a polynucleotide encoding a first lox site, a promoter and optionally an operator, a first reporter gene encoding a first reporter protein, an IRES, a first selection marker gene encoding a first selection maker protein and a second lox site, wherein the first lox site and the second lox site are different; (C) exchanging the first DNA cassette with a second DNA cassette, wherein the second DNA cassette comprises in 5′ to 3′ order a polynucleotide encoding the first lox site, a promoter, a second selection marker gene encoding a second selection marker protein, an IRES, a second reporter gene encoding a second reporter protein, a promoter and an optional operator, a Cas9 gene and the second lox site, wherein the first and second selection marker proteins are different and the first and second reporter proteins are different; (D) integrating a third DNA cassette comprising in 5′ to 3′ order a polynucleotide comprising a first Genomic Safe Harbor (GSH) homology arm containing an sgRNA (single guide RNA) target site, a third lox site, a third reporter gene encoding a third reporter protein, a forth lox site and a second GSH homology arm containing an sgRNA target site, wherein the first lox site, the second lox site, the third lox site and the forth lox site are different, wherein the first and second guide arms can contain at least one region with alterations (if needed to avoid recreating a targetable site), and wherein the third reporter protein is different from the second reporter protein and can be the same or different from the first reporter protein; and (E) exchanging the second DNA cassette with a fourth DNA cassette, wherein the fourth DNA cassette comprises in a 5′ to 3′ order a polynucleotide encoding the first lox site, a promoter, a fourth reporter gene encoding a fourth reporter protein, and the second lox site, wherein the fourth reporter protein is different from the third reporter protein and the second reporter protein and preferably different from the first reporter protein, thereby providing the cell with multiple Stable Integration Sites. Lox sites are the most commonly used type of RRS; however, different RRSs can be used as well.

The inventions further provide mammalian cells comprising a modified genomes, wherein a given genome is modified by insertion of at least three DNA cassettes within different regions of the genome, wherein the modified genome comprises (1) a first deoxyribonucleic acid sequence that is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of SEQ ID NOS: 1 and 2 prior to modification; (2) a second deoxyribonucleic acid sequence that is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of SEQ ID NOS: 5 to 10 prior to modification; and (3) a third deoxyribonucleic acid sequence that is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, 99% identical to at least one selected from the group consisting of SEQ ID NO: 11 and SEQ ID NO: 12 prior to modification, wherein the first deoxyribonucleic acid sequence is modified by insertion of a first DNA cassette, the second deoxyribonucleic acid sequence is modified by insertion of a second DNA cassette, and the third deoxyribonucleic acid sequence is modified by insertion of a third DNA cassette. The mammalian cells can each have (a) the first DNA cassette comprise a promoter and at least one selected from the group consisting of a selectable marker gene and a reporter gene; (b) the second DNA cassette comprise a promoter and at least one selected from the group consisting of a selectable marker gene and a reporter gene; and (c) the third DNA cassette comprise a promoter and at least one selected from the group consisting of a selectable marker gene and a reporter gene. Moreover, the mammalian cells can each have (a) the first DNA cassette comprise a promoter, a selectable marker gene and a reporter gene; (b) the second DNA cassette comprise a promoter, a selectable marker gene and a reporter gene; and (c) the third DNA cassette comprises a promoter, a selectable marker gene and a reporter gene. The first deoxyribonucleic acid sequence comprises a Stable Integration Site, and a gene of interest inserted therein. The gene of interest can encode a polypeptide of interest selected from the group consisting of antibodies, antibody chains, receptors, Fc-containing proteins, trap proteins, enzymes, factors, repressors, activators, ligands, reporter proteins, selection proteins, protein hormones, protein toxins, structural proteins, storage proteins, transport proteins, neurotransmitters and contractile proteins. The mammalian cells can be human cells and the first deoxyribonucleic acid sequence is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to SEQ ID NO: 1. Alternatively, the mammalian cell can be a CHO cell and the first deoxyribonucleic acid sequence is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to SEQ ID NO: 2. The first deoxyribonucleic acid sequence can comprise a Stable Integration Site produced using a guide sequence selected from the group consisting of SEQ ID NOS: 13 to 419. Additionally, the first deoxyribonucleic acid sequence can comprise a Stable Integration Site produced by using a guide sequence that binds to and/or is complementary to target sequences in SEQ ID NO: 2 at nucleotide position ranges selected from the group consisting of: (a) 1 to 2000; (b) 2001 to 4000; (c) 4001 to 6000; (d) 6001 to 8000; (e) 8001 to 10,000; (f) 10,001 to 12,000; (g) 12,001 to 14,000; (h) 14,001 to 16,000; (i) 16,001 to 18,000; (j) 18,001 to 20,000; (k) 20,001 to 22,000; (l) 22,001 to 24,000; (m) 24,001 to 26,000; (n) 26,001 to 28,000; (o) 28,001 to 30,000; (p) 30,001 to 32,000; (q) 32,001 to 34,000; (r) 34,001 to 36,000; (s) 36,001 to 38,000; (t) 38,001 to 40,000; (u) 40,001 to 42,000; and (v) 42,001 to terminus (44,232).

Additionally, there are provided mammalian cells comprising a modified genomes, wherein a modified genome comprises a deoxyribonucleic acid sequence comprising an AAVS1-like region modified by insertion of at least one DNA cassette, and wherein a guide sequence selected from the group consisting of SEQ ID NOS: 13 to 419 that binds to and/or is complementary to a sense or antisense strand of the AAVS1-like region. The mammalian cells can further comprise a second deoxyribonucleic acid sequence that is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of SEQ ID NOS: 5 to 10 prior to modification; and a third deoxyribonucleic acid sequence that is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of SEQ ID NO: 11 and SEQ ID NO: 12 prior to modification, wherein the first deoxyribonucleic acid sequence is modified by insertion of a first DNA cassette, the second deoxyribonucleic acid sequence is modified by insertion of a second DNA cassette, and the third deoxyribonucleic acid sequence is modified by insertion of a third DNA cassette. The second deoxyribonucleic acid sequence is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of one selected from the group consisting of SEQ ID NOS: 5 to 10 prior to modification; and the third deoxyribonucleic acid sequence is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of SEQ ID NO: 11 and SEQ ID NO: 12 prior to modification. The first deoxyribonucleic acid sequence comprises a Stable Integration Site produced by using a guide sequence that binds to and/or is complementary to at least one target sequence having at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identity to SEQ ID NO:2 at nucleotide positions: (a) 1 to 2000; or (b) 2001 to 4000; or (c) 4001 to 6000; or (d) 6001 to 8000; or (e) 8001 to 10,000; or (f) 10,001 to 12,000; or (g) 12,001 to 14,000; or (h) 14,001 to 16,000; or (i) 16,001 to 18,000; or (j) 18,001 to 20,000; or (k) 20,001 to 22,000; or (l) 22,001 to 24,000; or (m) 24,001 to 26,000; or (n) 26,001 to 28,000; or (0) 28,001 to 30,000; or (p) 30,001 to 32,000; or (q) 32,001 to 34,000; or (r) 34,001 to 36,000; or(s) 36,001 to 38,000; or (t) 38,001 to 40,000; or (u) 40,001 to 42,000; or (v) 42,001 to 44,232.

There are also provided mammalian cells comprising a modified genome, wherein a modified genome comprises a Stable Integration Site in a AAVS1-like region, wherein the Stable Integration Site is produced by using a guide sequence that binds to and/or is complementary to at least one target sequence having at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identity to SEQ ID NO:2 at nucleotide positions: (a) 1 to 2000; or (b) 2001 to 4000; or (c) 4001 to 6000; or (d) 6001 to 8000; or (e) 8001 to 10,000; or (f) 10,001 to 12,000; or (g) 12,001 to 14,000; or (h) 14,001 to 16,000; or (i) 16,001 to 18,000; or (j) 18,001 to 20,000; or (k) 20,001 to 22,000; or (l) 22,001 to 24,000; or (m) 24,001 to 26,000; or (n) 26,001 to 28,000; or (o) 28,001 to 30,000; or (p) 30,001 to 32,000; or (q) 32,001 to 34,000; or (r) 34,001 to 36,000; or(s) 36,001 to 38,000; or (t) 38,001 to 40,000; or (u) 40,001 to 42,000; or (v) 42,001 to 44,232.

There further provided mammalian cells according to the preceding paragraph, further comprising a second deoxyribonucleic acid sequence that is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of SEQ ID NOS: 5 to 10 prior to modification; and a third deoxyribonucleic acid sequence that is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of SEQ ID NO: 11 and SEQ ID NO: 12 prior to modification, wherein the first deoxyribonucleic acid sequence is modified by insertion of a first DNA cassette, the second deoxyribonucleic acid sequence is modified by insertion of a second DNA cassette, and the third deoxyribonucleic acid sequence is modified by insertion of a third DNA cassette. The mammalian cell can have the second deoxyribonucleic acid sequence at least is 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of one selected from the group consisting of SEQ ID NOS: 5 to 10 prior to modification; and the third deoxyribonucleic acid sequence is at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, or 99% identical to at least one selected from the group consisting of SEQ ID NO: 11 and SEQ ID NO: 12 prior to modification.

Additionally, there are provided methods of producing proteins of interest, wherein the method comprises the steps of: (1) culturing the above mammalian cells; and (2) harvesting the protein of interest. There also are provided cells made according to any of the above methods, as well as methods of using the disclosed cells.

### BRIEF DESCRIPTION OF THE FIGURES

The below figures illustrate an exemplary progression and creation of intermediate cells useful for creating cells having Stable Integration Sites in different regions of the genome, and thereafter creating cells having Stable Integration Sites in different regions of the genome. These figures illustrate embodiments of the invention, and do not limit the inventions in any manner.

FIG. 1 schematically depicts the modification of a cell that has a polynucleotide encoding a repressor protein and a polyadenylation signal under transcriptional control of a promoter, wherein the polynucleotide is randomly inserted in the cell genome.

FIG. 2 schematically depicts the modification of the cell of FIG. 1 after a DNA cassette (1) is randomly or site-specifically inserted into the cell genome. DNA cassette (1) comprises flanking lox sites (1 and 2), a promoter, a reporter gene (1), an IRES, selection marker gene (1) and a polyadenylation signal. Instead of lox sites, other RRSs can be used as well.

FIG. 3 schematically depicts the modification of the cell of FIG. 2 wherein DNA cassette (1) is replaced by recombinase mediated cassette exchange with DNA cassette (2). DNA cassette (2) comprises flanking lox sites (1 and 2), a promoter, selection marker gene (2), an IRES and reporter gene (2) and polyadenylation signal, and a Cas9 gene with a second polyadenylation signal under control of a second promoter (an operator is optional). Instead of lox sites, other RRSs can be used as well.

FIG. 4 schematically depicts the modification of the cell of FIG. 3 that has a DNA cassette (3) comprising flanking Genomic Safe Harbor (GSH) homology arms, lox sites (3 and 4) and a reporter gene (3) with a polyadenylation signal under the control of a promoter inserted into the Genomic Safe Harbor. The insertion is a site-specific integration and creates a Stable Integration Site between Lox3 and Lox4. Instead of lox sites, other RRSs can be used as well.

FIG. 5 schematically depicts the modification of the cell of FIG. 4, wherein DNA cassette (2) is replaced by recombinase mediated cassette exchange with DNA cassette (4). DNA cassette (4) comprises flanking lox sites (1 and 2), reporter gene (4) and a polyadenylation signal under the control of a promoter. This exchange removes the Cas9 gene. Instead of lox sites, other RRSs can be used as well.

FIG. 6 schematically depicts an sgRNA plasmid used in Example 6.

FIG. 7 depicts plots of Example 6 showing green fluorescent protein positive populations (Q1) for No HDR Template (control), 104mer HDR Template, 401mer HDR Template and 1030mer HDR Template. GFP positive is the vertical axis and CFP positive is the horizontal axis.

FIG. 8 schematically depicts a mammalian cell (HEK293, for example) with stably integrated Cas9 gene flanked by Lox sites 3 and 4. The Cas9 gene is under the control of at least a promoter (not depicted). AAVS1 also is schematically depicted. Instead of lox sites, other RRSs can be used as well. Promoters are present 5′ of genes, but are not depicted.

FIG. 9A and FIG. 9B schematically depict targeting plasmids containing sgRNA target site, left homology arm (here a GSH homology arm) for insertion into a region, such as a Genomic Safe Harbor (here AAVS1), Lox 1 site, a reporter gene (color 1), Lox 2 site, a right homology arm (here a GSH homology arm) for insertion into a region, such as a Genomic Safe Harbor (here AAVS1). At the 3′ end, FIG. 9A schematically depicts a reporter gene (Color 2), and FIG. 9B schematically depicts at the 3′ end a negative selection gene (Negative Selection 1). Promoters and optionally other moieties (such as operators) are represented by arrows pointed in a 5′ to 3′ direction. Both plasmids insert color 1 into a region, such as a Genomic Safe Harbor. Instead of lox sites, other RRSs can be used as well.

FIG. 10 schematically shows the results after Cas9 mediated integration into the Genomic Safe Harbor (AAVS1) of the mammalian cell (HEK293, for example). Color 1 is flanked by Lox 1 and Lox 2. A gene of interest can replace color 1 via RMCE. When a targeting plasmid according to FIG. 9A is properly integrated, the cell will be color 1 positive and color 2 negative. When a targeting plasmid according to FIG. 9B is properly integrated, the cell will be color 1 positive and will be able to propagate because the negative selection gene is removed. Instead of lox sites, other RRSs can be used as well. Promoters and optionally other moieties (such as operators) are represented by arrows pointed in a 5′ to 3′ direction.

FIG. 11 schematically depicts the insertion of FIG. 10 in greater detail. The cellular genome, including AAVS1, flanks the insert and the 5′ and 3′ ends. Color 1 is flanked by Lox 1 and Lox 2. FIG. 11, left side indicates the location of 5′ genome primer and 3′ insertion primer used with 5′ junction PCR. FIG. 11, right side indicates the location of 5′ insertion primer and 3′ genome primer used with 3′ junction PCR. Instead of lox sites, other RRSs can be used as well. A promoter 5′ of the color 1 gene is depicted as a 5′ arrow.

FIG. 12 shows that correct size fragments are amplified in HEK 293 cells by the junction PCR schematically depicted in FIG. 11. Stable Cas9 targeted HEK293 cells and the 5′ junction and the 3′ junction are obtained and detected, which establish correct insertion.

FIG. 13 shows that correct size fragments are amplified in CHO cells by the junction PCR schematically depicted in FIG. 11. Stable Cas9 targeted CHO cells and the 5′ junction and the 3′ junction are obtained and detected, which establish correct insertion. Instead of lox sites, other RRSs can be used as well.

FIG. 14 schematically depicts an exemplary cell comprising three cassettes integrated into regions of the genome with flanking RRSs (here lox 1 and lox 2). Depending on the cell type, each of the three cassettes can be integrated into different Stable Integration Sites (for example, AAVS1-like) schematically depicted at position A, and other available sites (such as Stable Site 1 and Stable Site 2) schematically depicted at positions B and C. The reporter genes can be the same or different. The negative selection genes can be the same or different, but preferably the same. The cell can contain additional Stable Integration Sites and integrated cassettes according the teachings contained herein. Promoters are present 5′ of genes, but are not depicted.

FIG. 15 schematically depicts the modification of the cell of FIG. 14 at schematically depicted positions A, B and C. Three cassettes each comprise flanking RRSs (here lox 1 and lox 2), a gene of interest, a positive selection marker gene, and a reporter* gene. The positive selection marker genes can be the same or different, but preferably the same. The reporter* genes can be the same or different, but each must be different from any of the reporter genes in the cell of FIG. 14. The genes of interest can be the same or different. The cassettes of FIG. 14 are replaced by the cassettes of FIG. 15 by RMCE. The cell can contain additional Stable Integration Sites and integrated cassettes according the teachings contained herein. Promoters are present 5′ of genes, but are not depicted.

FIG. 16 is a bar graph comparing protein produced by a three-site CHO-K1 cell (A, B, and C) compared to a two-site CHO-K1 cell (B and C).

FIG. 17 schematically depicts an exemplary cell comprising four cassettes integrated into regions of the genome with flanking RRSs (here lox 1 and lox 2, or lox 3 and lox 4). Depending on the cell type, each of the four cassettes can be integrated into different Stable Integration Sites (and other available sites (such as Stable Site 1 and Stable Site 2), and schematically depicted as positions A and B (SISs) and C and D (Stable Sites 1 and 2). The reporter genes can be the same or different. The negative selection genes can be the same or different, but preferably the same. The cell can contain additional Stable Integration Sites and integrated cassettes according the teachings contained herein. Promoters are present 5′ of genes, but are not depicted.

FIG. 18 schematically depicts the modification of the cell of FIG. 17 at schematically depicted positions A, B, C and D. Four cassettes each comprise flanking RRSs (here lox 1 and lox 2, or lox 3 and lox 4), a gene of interest, a positive selection marker gene, and a reporter* gene. The positive selection marker genes can be the same or different, but preferably the same. The reporter* genes can be the same or different, but each must be different from any of the reporter genes in the cell of FIG. 17. The genes of interest can be the same or different. In this figure, there are two copies of Gene of Interest 1 and two copies of Gene of Interest 2. The cassettes of FIG. 17 are replaced by the cassettes of FIG. 18 by RMCE. The cell can contain additional Stable Integration Sites and integrated cassettes according the teachings contained herein. Promoters are present 5′ of genes, but are not depicted.

### DETAILED DESCRIPTION OF THE INVENTIONS

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform, such as having a sought rate, amount, degree, increase, decrease, or extent of expression, concentration, or time, as is apparent from the teachings contained herein. Thus, this term encompasses values beyond those simply resulting from systematic error. For example, “about” can signify values either above or below the stated value in a range of approx. +/−10% or more or less depending on the ability to perform.

“AAVS1” can be a Genomic Safe Harbor and refers to Adeno-associated virus integration site 1, and is reported to be located on human chromosome 19 in nature and contains approximately 4.7 kilobases. The AAVS1 locus can be used according to the inventions.

“AAVS1-like” refers to an AAVS1 homolog found in CHO cells, and is disclosed herein. An AAVS1-like region containing an AAVS1-like Genomic Safe Harbor (GSH) can be used according to the inventions. SEQ ID NO:2 is an example of an AAVS1-like region.

A “DNA cassette” or “cassette” is a type of nucleic acid moiety that comprises at least a promoter, at least one open reading frame and optionally a polyadenylation signal, for example an SV40 polyadenylation signal. Other nucleic acid moieties, such as operators, also are optional. A DNA cassette thus is a polynucleotide that comprises two or more shorter polynucleotides. A cassette can comprise one or more gene and promoters, enhancers, operators, repressors, transcription termination signals, ribosomal entry sites, introns and polyadenylation signals.

“COSMC” has reportedly been found in hamster cells. Homologs of a partial or whole COSMC locus are candidates for use according to the inventions.

“CCR5” refers to C-C chemokine receptor type 5 gene, and has been reportedly found in human, mouse and rat cells. Homologs of a partial or whole CCR5 locus are candidates for use according to the inventions.

“Genomic Safe Harbors” or “GSH” refers to sites in the cell genome that can accommodate insertions of polynucleotides, such as DNA cassettes, and permit the inserted polynucleotide to function and not pose an undue burden on a transformed cell. Accordingly, Genomic Safe Harbors are ideal locations for creating Stable Integration Sites for the insertions of DNA cassettes through the practice of the inventions. Genomic Safe Harbors that can be utilized herein include, but are not limited to, AAVS1 and AAVS1-like. Reported loci that are candidates include, but are not limited to, CCR5, COSMC and Rosa26.

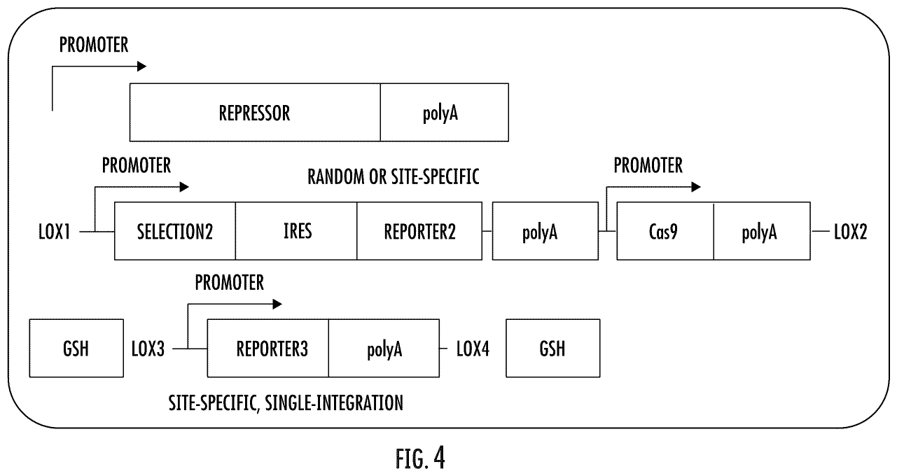

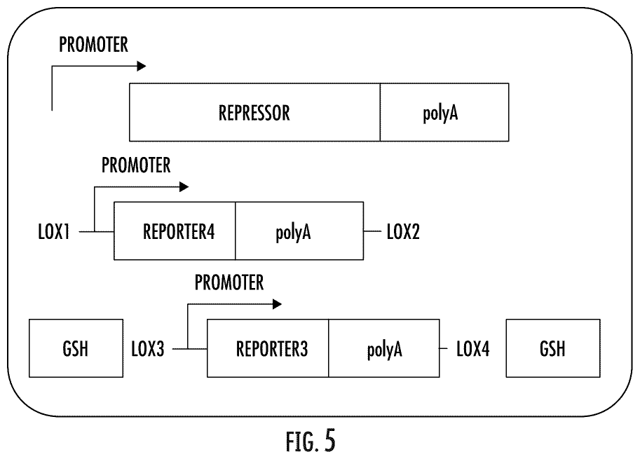

“Genomic Safe Harbor homology arm” or “GSH homology arms” is derived from Genomic Safe Harbors, and have homology to the Genomic Safe Harbor. Preferably, the Genomic Safe Harbor homology arm comprise about 100 to 2000 bases, more preferably about 300 to 1800 bases, more preferably about 400 to 1600 bases, more preferably about 500 to 1500 bases, more preferably about 500 to 1300 bases, more preferably about 500 to 1100 bases, more preferably about 500 to 1000 bases, more preferably about 600 to 1000 bases, more preferably about 700 to 1000 base, more preferably about 800 to 1000 bases, and still more preferably about 900 to 1000 bases. Typically, a polynucleotide to be inserted into a Genomic Safe Harbor will be flanked by a 5′ GSH Homology Arm and a 3′ GSH Homology Arm. For example, see FIGS. 4 and 5 showing a lox site-flanked DNA cassette that is further flanked by GSH Homology Arms.

“hRosa26” refers to the human homolog of the murine Rosa26 locus (“Reverse Orientation Splice Acceptor”). “Rosa26” refers to a partial or whole Rosa26 locus, and has been reportedly found in hamster cells in addition to mouse and human cells. Homologs of a partial or whole Rosa26 locus are candidates for use according to the inventions.

An “Intron” is a section of DNA located between exons. An intron is removed to form a mature messenger RNA. Preferred introns are those that can affect the starting point of translation, and exemplars are the hCMV-IE intron (Human cytomegalovirus immediate early protein) and FMDV intron (Foot and Mouth Disease Virus).

A “nucleic acid moiety” includes any arrangement of single stranded or double stranded nucleotide sequences. Nucleic acid moieties can include, but are not limited to, polynucleotides, promoters, enhancers, operators, repressors, transcription termination signals, ribosomal entry sites and polyadenylation signals.

“Operably linked” refers to one or more nucleotide sequences in functional relationships with one or more other nucleotide sequences. Such functional relationships can directly or indirectly control, cause, regulate, enhance, facilitate, permit, attenuate, repress or block an action or activity in accordance with the selected design. Exemplars include single-stranded or double-stranded nucleic acid moieties, and can comprise two or more nucleotide sequences arranged within a given moiety in such a way that sequence(s) can exert at least one functional effect on other(s). For example, a promoter operably linked to the coding region of a DNA polynucleotide sequence can facilitate transcription of the coding region. Other elements, such as enhancers, operators, repressors, transcription termination signals, ribosomal entry sites and polyadenylation signals also can be operably linked with a polynucleotide of interest to control its expression. Arrangements and spacing to achieve operable linkages can be ascertained by approaches available to the person skilled in the art, such as screening using western blots and RT-PCR.

“Operator” indicates a DNA sequence that is introduced in or near a polynucleotide sequence in such a way that the polynucleotide sequence may be regulated by the interaction of a molecule capable of binding to the operator and, as a result, prevent or allow transcription of the polynucleotide sequence, as the case may be. One skilled in the art will recognize that the operator must be located sufficiently in proximity to the promoter such that it is capable of controlling or influencing transcription by the promoter, which can be considered a type of operable linkage. The operator may be placed either downstream or upstream of the promoter. These include, but are not limited to, the operator region of the Lex A gene of E. coli, which binds the Lex A peptide and the lactose and 45 tryptophan operators, which bind the repressor proteins encoded by the Lad and trpR genes of E. coli. The bacteriophage operators from the lambda Pi and the phage P22 Mnt and Arc. Preferred operators are the Tet (tetracycline) operator (TetO or TO) and the Arc operator (ArcO or AO). Operators can have a native sequence or a mutant sequence. For example, mutant sequences of the Tet operator are disclosed in Wissmann et al., Nucleic Acids Res. 14: 4253-4266 (1986).

The Tet operator is preferred, and can be used to control transcription using a repressor, such as the Tetracycline repressor (TetR). Appropriate ligands for the repressor are tetracycline (tet), doxycycline (dox) and derivatives thereof. When the ligand binds to TetR, the affinity of the Tet repressor for the Tet operator is lessened and the Tet repressor separates from the operator, and thereby the operator becomes permissive for transcription. Other repressors can be paired for usage with their own respective operators.

The phrases “percent identity” or “% identical,” in their various grammatical forms, when describing a sequence is meant to include homologous sequences that display the recited identity along regions of contiguous homology, but the presence of gaps, deletions, or insertions that have no homolog in the compared sequence are not taken into account in calculating percent identity. As used herein, a “percent identity” or “% identical” determination between homologs would not include a comparison of sequences where the homolog has no homologous sequence to compare in an alignment. Thus, “percent identity” and “% identical” do not include penalties for gaps, deletions, and insertions.

A “homologous sequence” in its various grammatical forms in the context of nucleic acid sequences refers to a sequence that is substantially homologous to a reference nucleic acid sequence. In some embodiments, two sequences are considered to be substantially homologous if at least 50%-99%, 75%-99%, 85%-99%, 90%-99%, 95%-98%, 98%-99%, 50%, 55%, 60%, 65%, 70%, 75%, 80%, 85%, 90%, 91%, 92%, 93%, 94%, 95%, 96%, 97%, 98%, 99% or more of their corresponding nucleotides are identical over a relevant stretch of residues. In some embodiments, the relevant stretch is a complete (i.e., full) sequence.

“Polynucleotide” includes a sequence of nucleotides covalently joined, and includes RNA and DNA. Oligonucleotides are considered shorter polynucleotides. Genes are DNA polynucleotides (polydeoxyribonucleic acid) that ultimately encode polypeptides, which are translated from RNA (polyribonucleic acid) that was typically transcribed from DNA. DNA polynucleotides also can encode RNA polynucleotides that is not translated, but rather function as RNA “products”. The type of polynucleotide (that is, DNA or RNA) is apparent from the context of the usage of the term. A polynucleotide referred to or identified by the polypeptide it encodes sets forth and covers all suitable sequences in accordance with codon degeneracy. Polynucleotides, including those disclosed herein, include percent identity sequences and homologous sequences when indicated.

“Polypeptide” and “peptide” refers to sequence(s) of amino acids covalently joined. Polypeptides include natural, semi-synthetic and synthetic proteins and protein fragments. “Polypeptide” and “protein” can be used interchangeably. Oligopeptides are considered shorter polypeptides.

“Promoter” indicates a DNA sequence that cause transcription of a DNA sequence to which it is operably linked, i.e., linked in such a way as to permit transcription of the nucleotide sequence of interest when the appropriate signals are present and repressors are absent. The expression of a polynucleotide of interest may be placed under control of any promoter or enhancer element known in the art. A eukaryotic promoter can be operably linked to a TATA Box. The TATA Box is typically located upstream of the transcription start site.

Useful promoters that may be used include, but are not limited to, the SV40 early promoter region, SV40 E/L (early late) promoter, the promoter contained in the 3′ long terminal repeat of Rous sarcoma virus, the regulatory sequences of the metallothionein gene, mouse or human cytomegalovirus major immediate early (CMV-MIE) promoter and other CMV promoters, including CMVmin promoters. Plant expression vectors comprising the nopaline synthetase promoter region, the cauliflower mosaic virus 35S RNA promoter, and the promoter of the photosynthetic enzyme ribulose biphosphate carboxylase; promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I; insulin; immuno globulin; mouse mammary tumor virus; albumin; C.-feto protein; C.1-antitrypsin; 3-globin, and myosin light chain-2. Various forms of the CMV promoter can be used according to the inventions.

Minimal promoters, such as CMVmin promoters, can be truncated promoters or core promoters and are preferred for use in controlled expression systems. Minimal promoters and development approaches are widely known and disclosed in, for example, Saxena et al., Methods Molec. Biol. 1651:263-73 (2017); Ede et al., ACS Synth Biol. 5:395-404 (2016); Brown et al., Biotech Bioeng. 111:1638-47 (2014); Morita et al., Biotechniques 0:1-5 (2012); Lagrange et al., Genes Dev. 12:34-44 (1998). There are many CMVmin promoters described in the field.

“Protein of interest” or “polypeptide of interest” can have any amino acid sequence, and includes any protein, polypeptide, or peptide, and derivatives, components, domains, chains and fragments thereof. Included are, but not limited to, viral proteins, bacterial proteins, fungal proteins, plant proteins and animal (including human) proteins. Protein types can include, but are not limited to, antibodies, bi-specific antibodies, multi-specific antibodies, antibody chains (including heavy and light), antibody fragments, Fv fragments, Fc fragments, Fc-containing proteins, Fc-fusion proteins, receptor Fc-fusion proteins, receptors, receptor domains, trap and mini-trap proteins, enzymes, factors, repressors, activators, ligands, reporter proteins, selection proteins, protein hormones, protein toxins, structural proteins, storage proteins, transport proteins, neurotransmitters and contractile proteins. Derivatives, components, chains and fragments of the above also are included. The sequences can be natural, semi-synthetic or synthetic. Proteins of interest and polypeptides of interest are encoded by “genes of interest,” which also can be referred to as “polynucleotides of interest.” Where multiple genes (same or different) are integrated, they can be referred to as “first,” “second”, “third,” “fourth,” “fifth,” “sixth,” “seventh,” “eighth,” “ninth,” “tenth,” etc. as is apparent from the context of use.

“Recombinase recognition sites” (RRS), also known as “heterospecific recombination sites,” are used in recombinase mediated cassette exchange (RMCE). Cre/Lox, Dre/Rox, Vre/Vlox, SCre/Slox and Flp/Frt are suitable RRS systems, for example. Suitable RRSs for use according to the inventions include Lox P, Lox 66, Lox 71, Lox 511, Lox 2272, Lox 2372, Lox 5171, Lox M2, Lox M3, lox M7 and Lox M11. These sites can be referred to generically as first (1), second (2), third (3), fourth (4), fifth (5), sixth (6), seventh (7), eighth (8), ninth (9), tenth (10), etc., as is apparent from the context of usage. Cre/Lox is most commonly used RRS, but other RRSs can be used instead of Cre/Lox according to the inventions.

“Reporter proteins” as used herein, refers to any protein capable of generating directly or indirectly a detectable signal. Reporter proteins typically fluoresce, or catalyze a colorimetric or fluorescent reaction, and often are referred to as “fluorescent proteins” or “color proteins.” However, a reporter protein also can be non-enzymatic and non-fluorescent as long as it can be detected by another protein or moiety, such as a cell surface protein detected with a fluorescent ligand. A reporter protein also can be an inactive protein that is made functional through interaction with another protein that is fluorescent or catalyzes a reaction. Accordingly, any suitable reporter protein, as understood by one of skill in the art, could be used. In some aspects, the reporter protein may be selected from fluorescent protein, luciferase, alkaline phosphatase, β-galactosidase, β-lactamase, dihydrofolate reductase, ubiquitin, and variants thereof. Fluorescent proteins are useful for the recognition of gene cassettes that have or have not been successfully inserted and/or replaced, as the case may be. Fluid cytometry and fluorescence-activated cell sorting are suitable for detection. Examples of fluorescent proteins are well-known in the art, including, but not limited to Discosoma coral (DsRed), green fluorescent protein (GFP), enhanced green fluorescent protein (eGFP), cyano fluorescent protein (CFP), enhanced cyano fluorescent protein (eCFP), yellow fluorescent protein (YFP), enhanced yellow fluorescent protein (eYFP) and far-red fluorescent protein (e.g. mKate, mKate2, mPlum, mRaspberry or E2-crimson. See, for example, U.S. Pat. Nos. 9,816,110. Reporter proteins are encoded by polynucleotides, and are referred to herein as “reporter genes” or “reporter protein genes.” Reporter genes and proteins can be referred to generically as first (1), second (2), third (3), fourth (4), fifth (5), sixth (6), seventh (7), eighth (8), ninth (9), tenth (10), etc., as is apparent from the context of usage. Reporters can be considered a type of marker. “Color” or “fluorescent,” in their various grammatical forms, also can be used the more specifically refer to a reporter protein or gene.

A “repressor protein”, also referred to as a “repressor,” is a protein that can bind to DNA in order to repressor transcription, and is encoded by a polynucleotide, also referred to herein as a “repressor gene” or a “repressor proteins gene.” Repressors are of eukaryotic and prokaryotic origin. Prokaryotic repressors are preferred. Examples of repressor families include: TetR, LysR, LacI, ArsR, IcIR, MerR, AsnC, MarR, DeoR, GntR and Crp families. Repressor proteins in the TetR family include: ArcR, ActII, AmeR, AmrR, ArpR, BpeR, EnvR, EthR, HemR, HydR, IfeR, LanK, LfrR, LmrA, MtrR, Pip, PqrA, QacR, RifQ, RmrR, SimReg2, SmeT, SrpR, TcmR, TetR, TtgR, TrgW, UrdK, VarR YdeS, ArpA, BarA, Aur1B, CalR, CprB, FarA, JadR*, JadR2, MphB, NonG, PhIF, TyIQ, VanT, TarA, TyIP, BM1P1, Bm3R1, ButR, CampR, CamR, DhaR, KstR, LexA-like, AcnR, PaaRR, Psbl, Th1R, UidR, YDH1, Betl, McbR, MphR, PhaD, Q9ZF45, TtK, Yhgd, YixD, CasR, IcaR, LitR, LuxR, LuxT, OpaR, Orf2, SmcR, HapR, Ef0113, HlyIIR, BarB, ScbR, MmfR, AmtR, PsrA and YjdC proteins See Ramos et al., Microbiol. Mol. Biol. Rev., 69:326-56 (2005). Still other repressors include PurR, LacR, MetJ and PadR,

“Selectable” or “selection” marker proteins include proteins conferring certain traits, including but not limited to drug resistance or other selective advantages. Selection markers can give the cell receiving the selectable marker gene resistance towards a certain toxin, drug, antibiotic or other compound and permit the cell to produce protein and propagate in the presence of the toxin, drug, antibiotic or other compound, and are often referred to as “positive selectable markers.” Suitable examples of antibiotic resistance markers include, but are not limited to, proteins that impart resistance to various antibiotics, such as kanamycin, spectinomycin, neomycin, gentamycin (G418), ampicillin, tetracycline, chloramphenicol, puromycin, hygromycin, zeocin, and/or blasticidin. There are other selectable markers, often referred to as “negative selectable markers,” which cause a cell to stop propagating, stop protein production and/or are lethal to the cell in the presence of the negative selectable marker proteins. Thymidine kinase and certain fusion proteins can serve as negative selectable markers, including but not limited to GyrB-PKR. See White et al., Biotechniques, 50:303-309 (May 2011). Selectable marker proteins and corresponding genes (selectable marker genes) can be referred to generically as first (1), second (2), third (3), fourth (4), fifth (5), sixth (6), seventh (7), eighth (8), ninth (9), tenth (10), etc., as is apparent from the context of usage. In the figures, the selectable markers are positive selectable markers unless otherwise specified as a negative (neg.) marker.

“Single guide RNA” or “sgRNA” is used for targeting Cas9 to a site, and is usually 17-24 nucleotides long.

A “Stable Integration Site” or “SIS” is a region for site-specific integration of DNA polynucleotides of interest, including cassettes that comprise genes and/or other open reading frames, promoters and optionally other elements. Stable Integration Sites comprise an exogenously-sourced DNA cassette, and can be created according to the methods of the inventions described and depicted herein, preferably in a GSH. Constructs can be inserted into an SIS by a variety of approaches. Multiple Stable Integration Sites can be created and located on different chromosomes, different regions of the same chromosome or different positions in a same region of a chromosome.

A “Tetracycline Response Element” or “TRE” comprises seven copies of the 19 nucleotide TetO spaced apart by spacers comprising 17-18 nucleotides, and are commercially available. TetO sequences can vary and nucleotide substitutions are known. For example, altered sequences based on the Tet operator are disclosed in Wissmann et al., Nucleic Acids Res. 14: 4253-66 (1986). The spacers are not sequence specific. The spacers can be similar, but all should not be identical. A TRE is considered a type of operator as used herein.

All numerical limits and ranges set forth herein include all numbers or values thereabout or there between of the numbers of the range or limit. The ranges and limits described herein expressly denominate and set forth all integers, decimals and fractional values defined and encompassed by the range or limit. The ranges and limits described herein expressly denominate and set forth all integers, decimals and fractional values defined and encompassed by the range or limit. Thus, a recitation of ranges of values herein are merely intended to serve as a shorthand method of referring individually to each separate value falling within the range, unless otherwise indicated herein, and each separate value is incorporated into the specification as if it were individually recited herein.

### DETAILED DESCRIPTION

The inventions provide mammalian cells with multiple Stable Integration Sites, and are suitable for production of proteins of interest, including viral proteins, and the production of viral vectors, including adeno-associated virus vectors (AAV). One or more Stable Integration Sites can be within the Genomic Safe Harbor and one or more Stable Integration Sites can be outside of the particular Genomic Safe Harbor. Multiple Stable Integration Sites can be created and located on different chromosomes, different regions of the same chromosome or different positions in a same region of a chromosome.

Genomic Safe Harbors are discussed in Pellenz et al., Hum. Gene Therapy 30: 814-28 (2019); Papapetrou et al., Molecular Therapy 24:678-84 (2016).

Preferably, the Stable Integration Sites contain recognition sites to allow for Recombinase-Mediated Cassette Exchange (RMCE). Stable modification of cellular genomes can be undertaken with known approaches employing heterospecific recombination sites (also known as RRSs), such as Cre/Lox, Flp/Frt, transcription activator-like effector nuclease (TALEN), a TAL effector domain fusion protein, zinc finger nuclease (ZFN), a ZFN dimer, or a RNA-guided DNA endonuclease system, such as CRISPR/Cas9. See U.S. Pat. No. 9,816,110 at cols. 17-18; Sajgo et al., PLoS ONE 9: e91435 (2014); Suzuki et al., Nucl. Acids. Res. 39: e49 (2011) Integration using Bxb1 integrase in human, mouse and rat cells also can be undertaken. Russell et al., Biotechniques 40:460-64 (2006).

Recombinase recognition sites, also known as heterospecific recombination sites, are referred to generically as first (1), second (2), third (3), fourth (4), fifth (5), sixth (6), seventh (7), eighth (8), ninth (9), tenth (10), etc., as is apparent from the context of usage. Suitable Lox sites for use according to the inventions include, but are not limited to, Lox P, Lox 66, Lox 71, Lox 511, Lox 2272, Lox 2372, Lox 5171, Lox M2, Lox M3, lox M7 and Lox M11. Other RRSs can be used as well. Lox sites are the most commonly used type of RRS; however, different RRSs can be used as well.

Homology arms preferably start within about 10 to 20 bases, more preferably 10 to 15 bases, of the cut site. A greater distance can be used as well, but with lower efficiency. In order to ensure that the DNA cassette(s) inserted into the Genomic Safe Harbor(s) maintain stability in the event that the homology repair could possibly recreate a targetable site, as determined by the skilled person, the guide arm region of the DNA cassette can be made to contain alterations (for example, base mismatches) that disrupt the function of CRISPR target site. There are two approaches that can be employed independently or together. The first approach is to insert base substitutions to create base mismatches in the CRISPR twenty base target site or the protospacer adjacent motif (PAM), which is usually 2 to 6 bases. The second approach is to create a donor plasmid where insertion divides the CRISPR target site or divides the CRISPR target site from the PAM.

Human cell lines include amniotic cells (such as Human Amniotic Epithelial cells), Hela cells, Per.C6 cells and HEK 293 cells. Examples of HEK 293 cells include, but are not limited, to HEK 293, HEK 293A, HEK 293E, HEK 293F, HEK 293 FT, HEK 293FTM, HEK 293H, HEK 293MSR, HEK 293S, HEK 293SG, HEK 293SGGD, HEK 293T and mutants and variants thereof. Rodent cell lines, such as Sp2/0 cells, BHK cells and CHO cells and mutants and variants thereof, also can be used according to the inventions. CHO cells include, but are not limited to, CHO-ori, CHO-K1, CHO-s, CHO-DHB11, CHO-DXB11, CHO-K1SV, and mutants and variants thereof.

The mammalian cells of the inventions are produced by advantageously producing and utilizing a cell intermediate that has a cassette comprising a Cas9 endonuclease gene flanked by recombinase recognition sites and integrated into the genome via RCME. Without being bound by any theory, the inventive use of an integrated Cas9 gene when expressed appears to increase the efficiency of homology arm integration into Genomic Safe Harbors by increasing the occurrence of cuts in genomic DNA caused by the Cas9 endonuclease. The use of stably integrated Cas9 gene of the inventions provides 10, 10², 10³, 10⁴, 10⁵, 10⁶, 10⁷, 10⁸, 10⁹, or 10¹⁰ greater HDR efficiency than HDR without a stably integrated Cas9 gene. Ultimately, this intermediate cell can be further subjected to RMCE to remove the cassette containing the Cas9 gene.

As a starting point for engineering of cells, polynucleotide sequences of interest, as well as the operably linked promoter and optional operators, may be introduced into the cell by transfection of a plasmid containing said polynucleotide sequences and elements. Accordingly, the inventions include the generation of cells as described.

Suitable plasmid constructs can be made by those of skill in the art. Useful regulatory elements, described previously or known in the art, can also be included in the plasmid constructs used to transfect the cells. Some non-limiting examples of useful regulatory elements include, but are not limited to, promoters, enhancers, sequences encoding suitable mRNA ribosomal binding sites, and sequences that control the termination of transcription and translation. Suitable plasmid constructs also may comprise non-transcribed elements such as an origin of replication, other 5′ or 3′ flanking non-transcribed sequences, and 5′ or 3′ non-translated sequences such as splice donor and acceptor sites. One or more selectable marker genes may also be incorporated. Useful selectable marker proteins and reporter proteins for use with the present inventions are known and can be readily identified by those of skill in the art.

A plasmid construct encoding a gene of interest may be delivered to the cell using a viral vector or via a non-viral method of transfer.

Non-viral methods of nucleic acid transfer include naked nucleic acid, liposomes, and protein/nucleic acid conjugates. A plasmid construct that is introduced to the cell may be linear or circular, may be single-stranded or double-stranded, and may be DNA, RNA, or any modification or combination thereof.

A plasmid construct may be introduced into the cell by transfection. Those of skill in the art are aware of numerous different transfection protocols, and can select an appropriate system for use in transfecting cells. Generally, transfection methods include, but are not limited to, viral transduction, cationic transfection, liposome transfection, dendrimer transfection, electroporation, heat shock, nucleofection transfection, magnetofection, nanoparticles, biolistic particle delivery (gene gun), and proprietary transfection reagents such as Lipofectamine, Dojindo Hilymax, Fugene, jetPEI, Effectene, or DreamFect.

The inventions are further described by the following Examples, which are illustrative of the many embodiments and aspects of the invention, but do not limit the inventions in any manner. In the Examples, the selectable markers are positive selectable markers unless otherwise specified as a negative (neg.) marker.

### Example 1

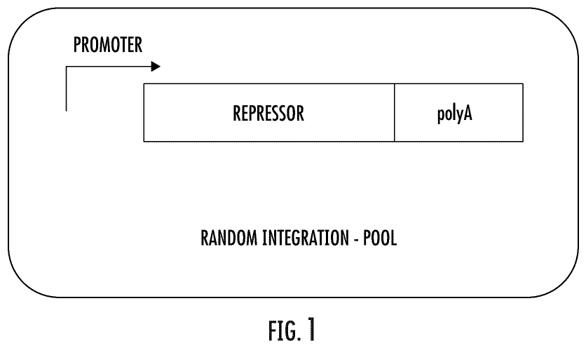

This example concerns the creation of mammalian cells comprising a repressor, such as TetR, under control of a promoter, such as a CMV promoter. See FIG. 1. The cell is transfected with a polynucleotide comprising the promoter and the repressor gene. The polynucleotide is randomly inserted into the cell genome. Western blots and Taqman can be used in the cell pool to identify transformants and determine average copy number. The integration of a repressor, such as TetR, allows for control of transcription of polynucleotides that under control of a promoter and an operator.

### Example 2

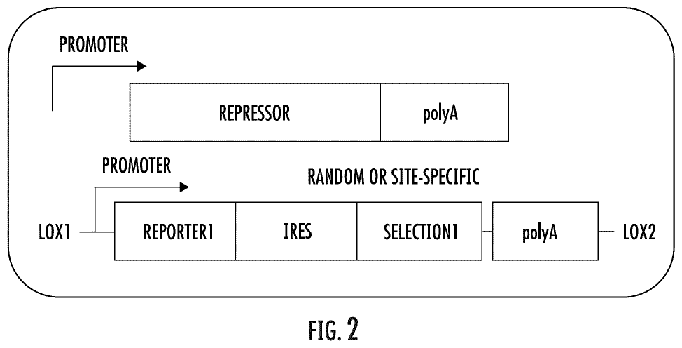

This example concerns further engineering of the cells of Example 1. DNA cassette 1 is schematically depicted in FIG. 2 and comprises flanking lox sites (1 and 2) and further comprises in 5′ to 3′ order a promoter, reporter gene (1) encoding reporter protein (1), an IRES and selection marker gene (1) encoding selection marker protein (1) and a polyadenylation signal. DNA cassette (1) optionally can include an operator operably linked to the promoter. DNA cassette (1) is randomly or site-specifically inserted into the cell genome. The first lox site and the second lox site on DNA cassette (1) are different.

Where the tet operator is used in DNA cassette (1), multiple rounds of −ligand/+ligand sort and single cell sort will identify Lox-site stable cells for dox-regulated expression. Thus, when the ligand, such as doxycycline or tetracycline, is present, TetR will not bind to the operator, and thereby conditions are permissive for transcription of reporter gene (1) and selection marker polynucleotide (1).

### Example 3

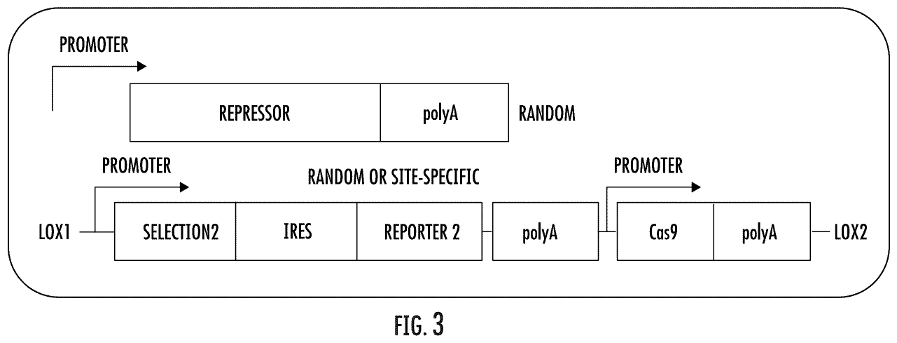

In this example, RMCE is performed to replace DNA cassette (1) with DNA cassette (2) in the cells of Example 2. As schematically depicted in FIG. 3, DNA cassette (2) comprises flanking lox sites (1 and 2), and further comprises in 5′ to 3′ order a promoter, selection marker gene (2) encoding selection marker protein (2), an IRES and reporter gene (2) encoding reporter protein (2), and a Cas9 gene under control of a second promoter (optionally operably linked to an operator).

In an embodiment, a CMV promoter is operably linked to a tet operator to control transcription of the Cas9 gene. When the cells are in the presence of doxycycline or tetracycline, TetR is no longer able to bind the tet operator, and thus allow transcription of the Cas9 gene to occur. Reporter protein (1) is different from reporter protein (2), and selection marker protein (1) is different from selection marker protein (2).

### Example 4

This example concerns the integration of DNA cassette (3) into a Genomic Safe Harbor. See FIG. 4. DNA cassette (3) comprises in 5′ to 3′ order a polynucleotide comprising a first Genomic Safe Harbor homology arm containing an sgRNA target site, lox site (3), a promoter operably linked to reporter gene (3) encoding reporter protein (3), a polyadenylation signal, lox site (4) and a second Genomic Safe Harbor homology arm containing an sgRNA target site, wherein the first and second guide arm target sites each can contain a region with alterations if needed to avoid recreating a targetable site. Lox site (1), lox site (2), lox site (3), and lox site (4) are different from one another. Reporter protein (3) is different from reporter protein (2). Reporter protein (3) and reporter protein (1) can be the same or different. Homology arms of about 1000 bases are used in this example.

When the Cas9 endonuclease is expressed, the efficiency of DNA cassette 3 integration is increased. Without being bound by any theory, the inventive use of an integrated Cas9 gene appears to increase the efficiency of integration by increasing the occurrence of cuts in genomic DNA caused by the Cas9 endonuclease. The use of stably integrated Cas9 gene of the inventions provides 10, 10², 10³, 10⁴, 10⁵, 10⁶, 10⁷, 10⁸, 10⁹, or 10¹⁰ greater HDR efficiency than HDR without a stably integrated Cas9 gene.

If needed, alterations in the first and second Genomic Safe Harbor homology arms ensue that the DNA cassette (3) will stay integrated by avoiding recreation of a targetable site. The smaller cassette therein, namely the region between lox site (3) and lox site (4), is available for RMCE and is referred to as a Stable Integration Site.

### Example 5

This Example concerns the final form of the cell line, and is schematically depicted in FIG. 5. To ensure stability of a cell line over time, it is preferred to remove the Cas9 gene. Accordingly, DNA cassette (2) is replaced by RMCE with DNA cassette (4), and removes the Cas9 gene. DNA cassette (4) comprises flanking lox sites (1 and 2) and reporter gene (4) encoding reporter protein (4) under the control of a promoter. Reporter protein (4) is different from reporter protein (2) and reporter protein (3) and preferably different from reporter protein (1).

The resulting cells will have two integration sites within the genome, one integration site within a Genomic Safe Harbor (for example, a Stable Integration Site) and one integration site outside of that particular Genomic Safe Harbor. It is possible to create still further integration sites by applying the approaches described above, including the use of an integrated Cas9 gene and the use of additional and different GSH homology arms.

### Example 6

This example is a comparison of the efficiency of using Cas9 with homology directed repair (HDR) as disclosed herein compared to conventional HDR. As reported in the literature, HDR is precise, but desired recombinational events occur infrequently: 1 in 10⁶-10⁹ cells (0.0001% to 0.0000001%). Hsu et al., Cell 157: 1262-78 (2014).

In order to assess the advantages of a stably integrated Cas9 gene, a CHO cell having the sites disclosed in U.S. Pat. No. 7,771,997 (“Stable Site 1”) and U.S. Pat. No. 9,816,110 (“Stable Site 2”) was modified. Regeneron provides a suite of goods and services referred to as EESYR®. CHO cells with integrated sequences in Stable Site 1 and Stable Site 2 are disclosed in US 2019/0233544 A1, and each is referred to as an “enhanced expression locus” therein. Sequences set forth in these patents and Examples 11 and 12 can be used according to the inventions described and depicted herein.

A CHO cell was modified to include a cyano fluorescent protein reporter gene under control of a promoter in Stable Site 1, and a selection marker gene and a yellow fluorescent protein reporter gene under the control of the same promoter in Stable Site 2. Additionally, a Cas9 gene under control of a second promoter with an operator also was inserted into Stable Site 2. The Cas9 gene can be eventually removed in accordance with the teachings contained herein.

The cyano fluorescent protein can be change to fluoresce green by changing the tyrosine residue at position 66 to tryptophan. The sgRNA Delivery Plasmid comprise a selection marker (Ampicillin resistance), a POL III promoter (RNA Polymerase III promoter), a target sequence and gRNA scaffold, a POL III Terminator and Digest Sites 1 and 2. Pol III promoters include H1 and U6.

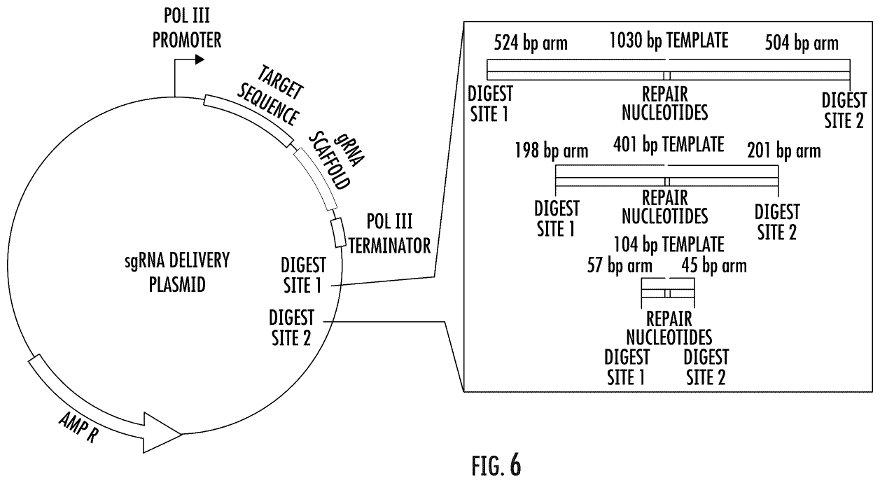

As depicted in FIG. 6, sgRNA delivery plasmids were constructed containing HDR Templates: a 104mer insert (having a 57 bp arm and a 45 bp arm), a 401mer insert (having a 198 bp arm and a 201 bp arm) or a 1030mer (having a 524 bp arm and a 504 bp arm) insert containing homology arms and the sequence to effect the change from cyano to green, which in this example was composed of 2 nucleotides (“repair nucleotides”). The HDR templates were inserted into the Digest Sites (for example, NotI and/or other appropriate sites) of the sgRNA delivery plasmid to form a sgRNA target plasmid. A sgRNA delivery plasmid without an insert (No HDR Template) was used as a control

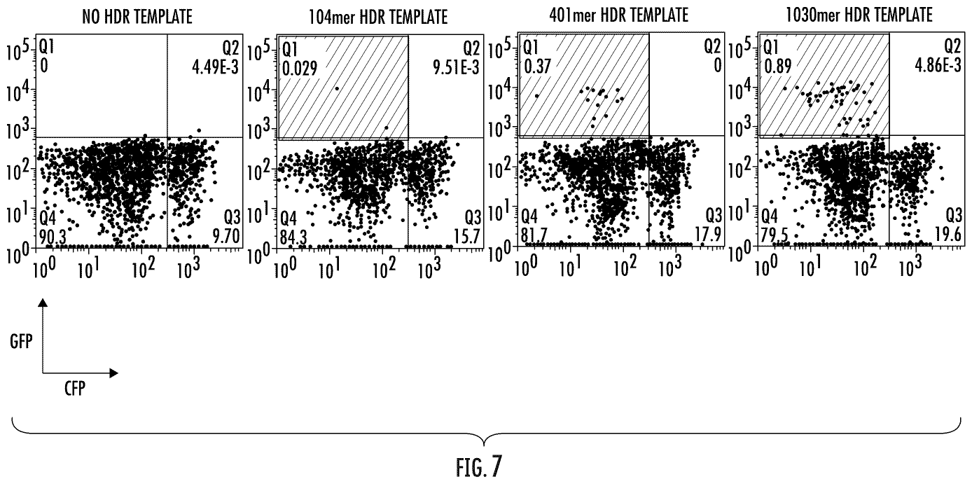

FIG. 7 shows that the control exhibited no green positives in Q1. The cells with HDR Template exhibited green positives in Q1, and the green positive population in Q1 consistently increased with the increased size of the HDR Template (left to right). The cells with the 1030mer HDR Templates showed the greatest efficiency in repair, which was about 6.5 percent.

The cells of this example possesses Stable Site 1 and Stable Site 2 and the SIS created in a GSH according to the inventions. Thus, this cell possess three sites for stable integration of genes of interest.

### Example 7—Generation of an Intermediate Human Cell Comprising a Stable Integration Site in a Genomic Safe Harbor (AAVS1)

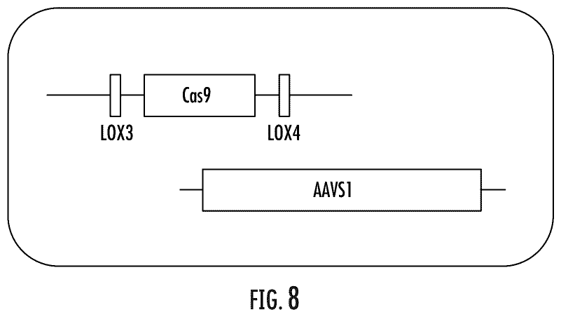

In this example, the starting point is HEK293 cell with stably integrated Cas9 gene flanked by Lox sites 3 and 4. The Cas9 gene is under the control of at least a promoter (not depicted). AAVS1 also is schematically depicted. See FIG. 8. This cell can made according to Examples 1~4 and FIGS. 1-4.

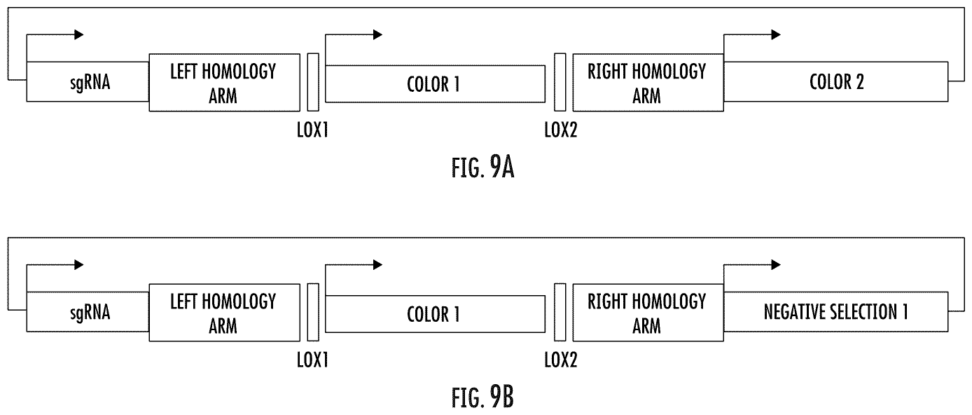

Targeting plasmids containing sgRNA target site, left homology arm (here a GSH homology arm) for insertion into a region, such as a Genomic Safe Harbor (here AAVS1), Lox 1 site, a reporter gene (color 1), Lox 2 site, a right homology arm (here a GSH homology arm) for insertion into a region, such as a Genomic Safe Harbor (here AAVS1). See FIGS. 9A and 9B for alternative targeting plasmids. At the 3′ end, one targeting plasmid has reporter gene (Color 2), See FIG. 9A. The other targeting plasmid has at the 3′ end a negative selection gene (Negative Selection 1). See FIG. 9B. Promoters and optionally other moieties (such as operators) are represented by arrows pointed in a 5′ to 3′ direction in FIG. 9A and FIG. 9B. Both plasmids insert color 1 into a region, such as a Genomic Safe Harbor (here AAVS1).

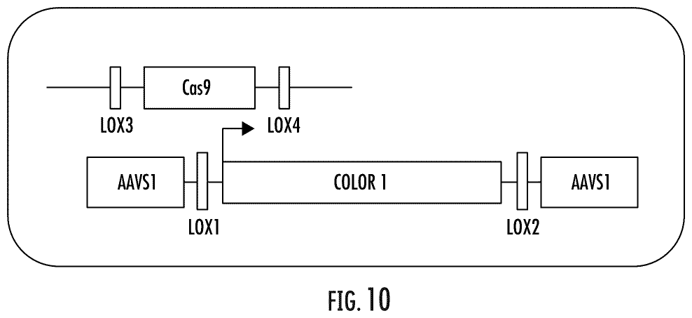

Cas9 mediated integration of a targeting plasmid (for example, FIG. 9A or FIG. 9B) into the Genomic Safe Harbor (AAVS1) of the HEK293 cell is schematically depicted in FIG. 10. Color 1 is flanked by Lox 1 and Lox 2. A gene of interest can replace color 1 via RMCE.

When a targeting plasmid according to FIG. 9A is properly integrated, the cell will be color 1 positive and color 2 negative. When a targeting plasmid according to FIG. 9B is properly integrated, the cell will be color 1 positive and will be able to propagate because the negative selection gene is removed. This cell is considered an intermediate. Ultimately, the cell can be further subjected to RMCE at lox sites 3 and 4 to remove the cassette containing the Cas9 gene, as shown in FIG. 8. See, for example, Example 5.

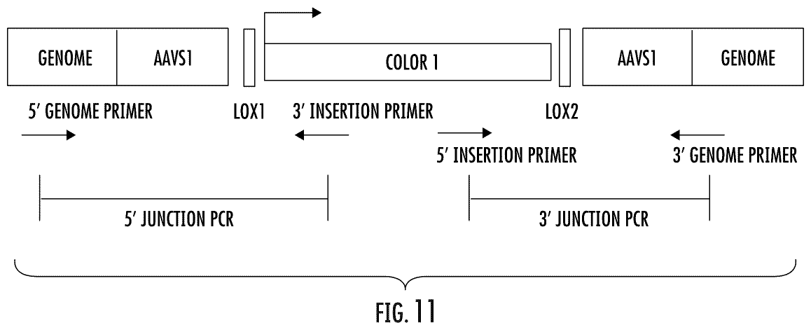

The precision of this inventive methodology is shown in FIGS. 10 and 11. FIG. 11 depicts the insertion of FIG. 10 in greater detail. The cellular genome, including AAVS1, flanks the insert and the 5′ and 3′ ends. Color 1 is flanked by Lox 1 and Lox 2. FIG. 11, left side identifies the location of 5′ genome primer and 3′ insertion primer used with 5′ junction PCR. FIG. 11, right side identifies the location of 5′ insertion primer and 3′ genome primer used with 3′ junction PCR.

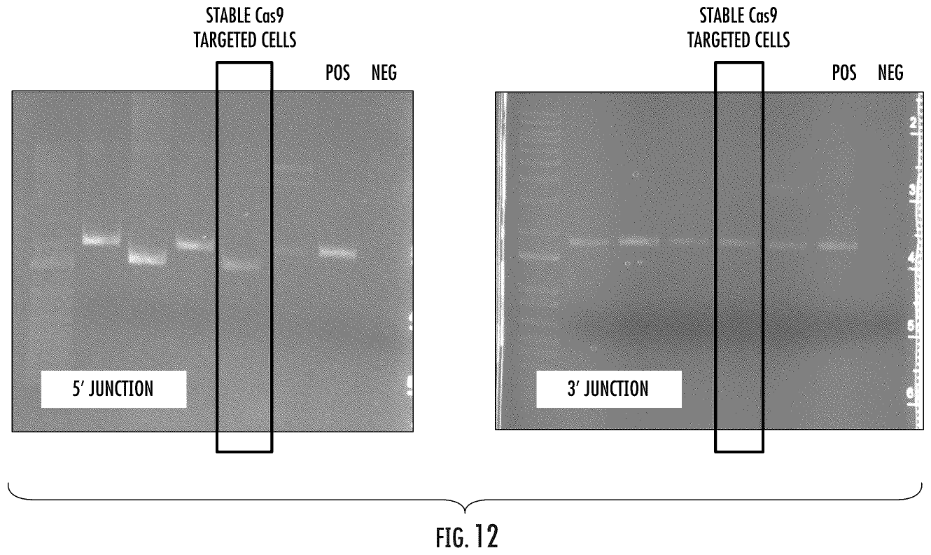

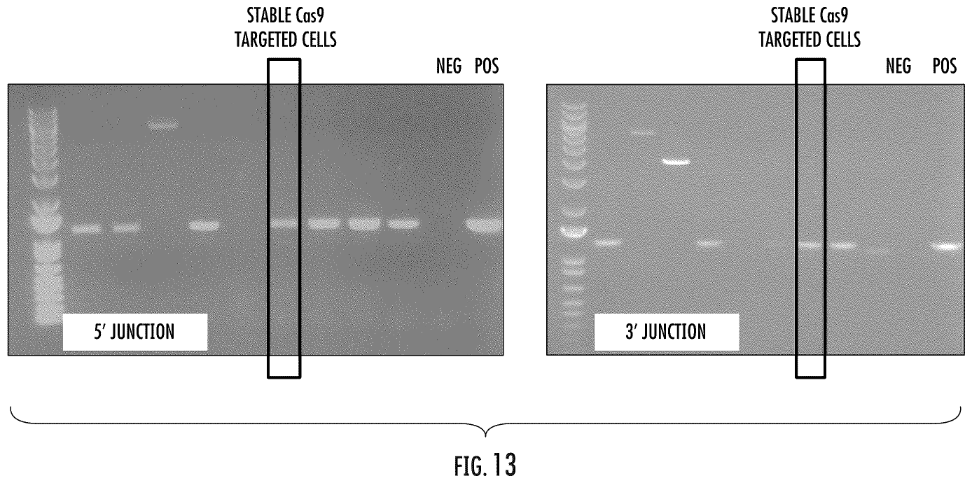

Junction PCR shows that correct size fragments are amplified and labeled as “Stable Cas9 targeted cells.” See FIGS. 12 and 13. Stable Cas9 targeted cells and the 5′ junction and the 3′ junction are obtained and detected, which establish correct insertion. Positive and negative controls are at the right hand columns of each gel.

### Example 8—CHO Regions and Sequences

For CHO cells, the sequences set forth in U.S. Pat. No. 7,771,997 (Stable Site 1) and U.S. Pat. No. 9,816,110 (Stable Site 2) can be utilized. The sequences and homologous sequences within the percent identity values of U.S. Pat. Nos. 7,771,997 and 9,816,110 are hereby incorporated by reference. An AAVS1-like region disclosed herein can be used to create Stable Integration Sites according to the inventions.

Candidate loci for use according to the inventions are reported in the literature. Hamaker and Lee, Curr. Op. Chem. Eng. 22:152-60 (2018) identify 30 hot spot loci. Hilliard and Lee, Biotech. Bioeng. 118:659-75 (2021) sought to identify safe harbor regions in CHO using an epigenomic analysis for Hi-C stable regions, and found an overlap with 5 of the 30 regions identified by Hamaker and Lee. See Supplementary Table 3 of Hilliard and Lee. Gaidukov et al., Nucl. Acids Res. 46:4072-86 (2018) also identifies loci for integration in CHO cells, including a putative Rosa26. Lee et al., Scientific Reps. 5:8572 (2015) reported a COSMC locus in hamster cells. In sum, these papers identify several unannotated regions and gene regions in CHO, and the gene regions are set forth below:

| | | | | |
|---|---|---|---|---|
| BMP5 | SSBP2 | TRMT6 | CLCC1 | FAM114A1 |
| | | | | (NOXP20) |
| LRBA | DCN | CEP128 | AACS | ALDH5A1 |
| SMAD6 | PTPRQ | ROSA26 | ADGRL4 | GPM6A |
| K1AA1551 | HPRT | CLCN3 | FER1L4 | COSMC |
| (C12ORF35) | | | | |

### Example 9—CHO Cells with Three or More Insertion Sites

CHO cells containing multiple insertion cites using the cells disclosed in US 2019/0233544 A1. Stable Site 1 and Stable Site 2 can be used initially in accordance with the teachings contained herein that utilize an integrated Cas9 gene. Once one or more Stable Integration Sites are created in Genomic Safe Harbors, such as in the AAVS1-like region (see, for example, SEQ ID NO:2) and counterpart guide sequences (see, for example, SEQ ID NOS: 13 to 419). Guide sequences can bind to target sequences in SEQ ID NO:2 at nucleotide position ranges selected from the group consisting of: (a) 1 to 2000; (b) 2001 to 4000; (c) 4001 to 6000; (d) 6001 to 8000; (e) 8001 to 10,000; (f) 10,001 to 12,000; (g) 12,001 to 14,000; (h) 14,001 to 16,000; (i) 16,001 to 18,000; (j) 18,001 to 20,000; (k) 20,001 to 22,000; (l) 22,001 to 24,000; (m) 24,001 to 26,000; (n) 26,001 to 28,000; (o) 28,001 to 30,000; (p) 30,001 to 32,000; (q) 32,001 to 34,000; (r) 34,001 to 36,000; (s) 36,001 to 38,000; (t) 38,001 to 40,000; (u) 40,001 to 42,000; and (v) 42,001 to 44,232.

Stable Site 1 and Stable Site 2 of U.S. Pat. Nos. 7,771,997 and 9,816,110 can be used for expression of genes of interest to encode proteins of interest. Cells with SISs ultimately can have 3, 4, 5, 6, 7, 8, 9, 10 or more sites for expressing genes of interest.

Preferably, a CHO cell comprising Stable Sites 1 and 2 is modified to create a third site in a Genomic Safe Harbor, namely a Stable Integration Site. Preferred Genomic Safe Harbors for creation of such a CHO cell are in the AAVS1-like region. Other CHO cell types can be used to create multiple sites according to the teachings contained herein.

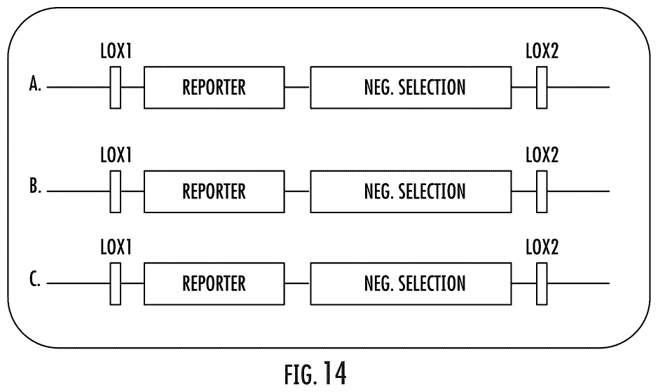

FIG. 14 schematically depicts an exemplary cell comprising three cassettes integrated into regions of the genome with flanking RRSs (here lox 1 and lox 2). Depending on the cell type, each of the three cassettes can be integrated into different Stable Integration Sites and other available sites (such as Stable Site 1 and Stable Site 2) schematically depicted as positions A, B and C. The reporter genes can be the same or different. The negative selection genes can be the same or different, but preferably the same. The cell can contain additional Stable Integration Sites and integrated cassettes according the teachings contained herein.

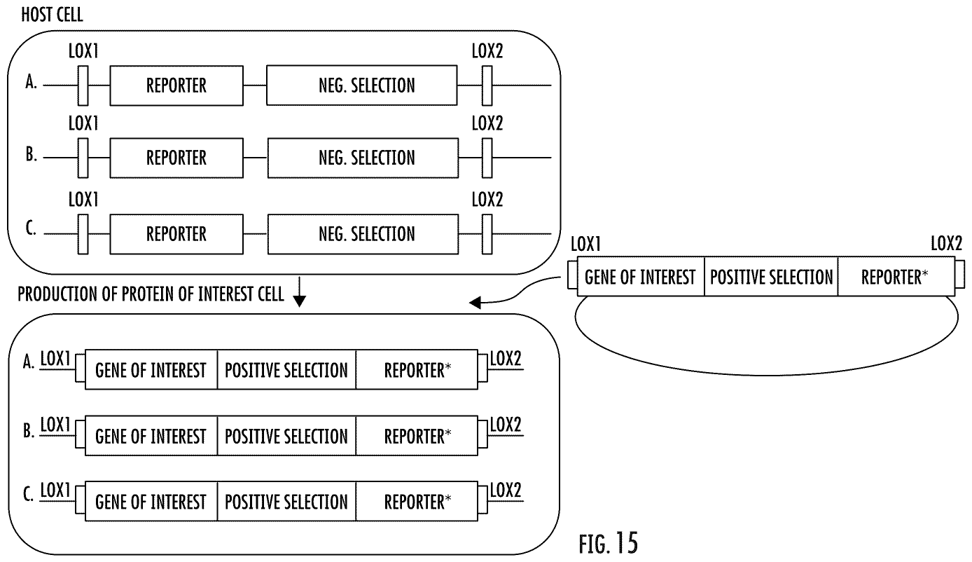

FIG. 15 schematically depicts the modification of the cell of FIG. 14 at schematically depicted positions A, B and C. Three cassettes each comprise flanking RRSs (here lox 1 and lox 2), a gene of interest, a positive selection marker gene, and a reporter* gene. The positive selection marker genes can be the same or different, but preferably the same. The reporter* genes can be the same or different, but each must be different from any of the reporter genes in the cell of FIG. 14. The genes of interest can be the same or different. The cassettes of FIG. 14 are replaced by the cassettes of FIG. 15 by RMCE. The cell can contain additional Stable Integration Sites and integrated cassettes according the teachings contained herein.

The combination of negative and positive selection assures isolation of cells that underwent recombination in all sites. If the gene of interest is the same in each of the three cassettes, the cell can result in high yield protein expression. For example, 7, 8, 9, 10 or more grams per liter (g/l) of protein production is possible.

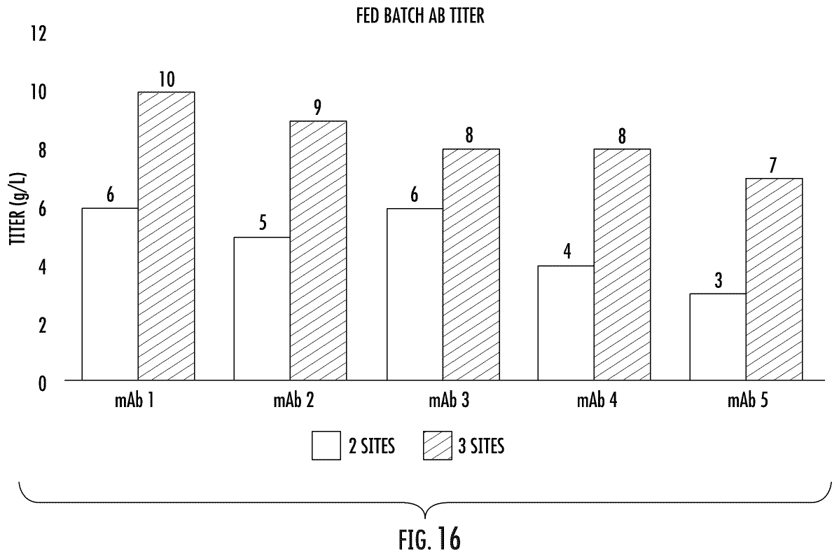

FIG. 16 shows the results from five different human IgG antibodies that were stably integrated using Cre-lox recombination into CHO K1 derived hosts engineered with either 2 integration sites (Stable Site 1 and 2) or 3 integration sites (Stable Site 1, Stable Site 2 and AAVS1-like (see SEQ ID NO:2)). Isogenic cell lines (ICLs) were isolated using flow cytometry. Fed batch production of ICLs were inoculated into chemically defined production media, and production cultures were carried out for 13 days. Antibody titer in conditioned media was determined using a protein A HPLC based method, and each three-site cell expressing a given antibody (1, 2, 3, 4, or 5) expressed a greater amount of protein than the comparison two-site cell. The three-site cell can provide increases of 10%, 20%, 30%, 40%, 50%, 60%, 70%, 80%, 90%, 100%, 110%, 120%, 130%, 140%, 150% or more over the two-site cell.

Alternatively, different genes of interest can be used in the cassettes. For example, heavy chain and light chain sequences of an antibody can be gene of interest.

Turning to a four-site cell, preferably a CHO cell comprising Stable Site 1 and 2 is modified to create a third and fourth site in a Genomic Safe Harbor, namely a Stable Integration Site. Preferred Genomic Safe Harbors for creation of such a CHO cell are in the AAVS1-like region, which can be the third site. A fourth site can be created in other loci, including but not limited to:

| | | | | |
|---|---|---|---|---|
| BMP5 | SSBP2 | TRMT6 | CLCC1 | FAM114A1 |
| | | | | (NOXP20) |
| LRBA | DCN | CEP128 | AACS | ALDH5A1 |
| SMAD6 | PTPRQ | ROSA26 | ADGRL4 | GPM6A |
| K1AA1551 | HPRT | CLCN3 | FER1L4 | COSMC |
| (C12ORF35) | | | | |

Other CHO cell types can be used to create multiple sites according to the teachings contained herein.

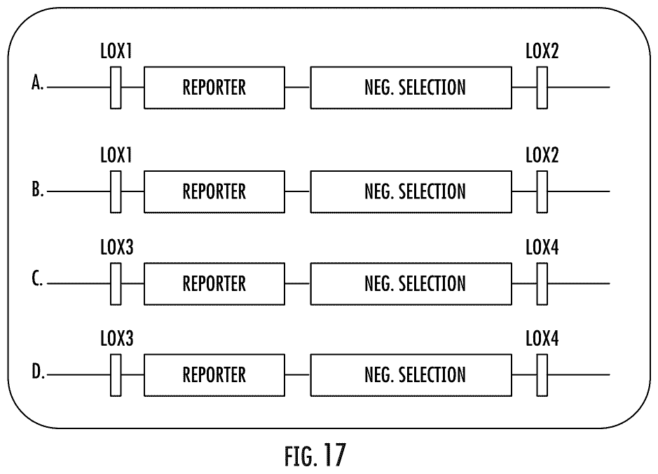

FIG. 17 schematically depicts an exemplary cell comprising four cassettes integrated into regions of the genome with flanking RRSs (here lox 1 and lox 2, or lox 3 and lox 4). Depending on the cell type, each of the four cassettes can be integrated into different Stable Integration Sites and other available sites (such as Stable Site 1 and Stable Site 2) schematically depicted as positions A, B, C and D. The reporter genes can be the same or different. The negative selection genes can be the same or different, but preferably the same. The cell can contain additional Stable Integration Sites and integrated cassettes according the teachings contained herein.

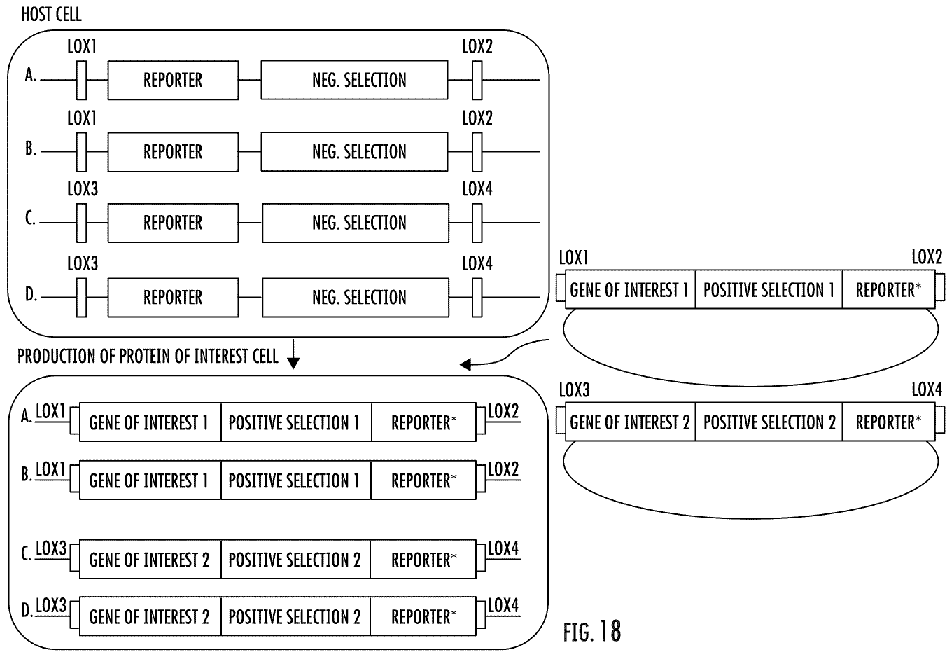

FIG. 18 schematically depicts the modification of the cell of FIG. 17 at schematically depicted positions A, B, C and D. Four cassettes each comprise flanking RRSs (here lox 1 and lox 2, or lox 3 and lox 4), a gene of interest, a positive selection marker gene, and a reporter* gene. The positive selection marker genes can be the same or different, but preferably the same. The reporter* genes can be the same or different, but each must be different from any of the reporter genes in the cell of FIG. 17. The genes of interest can be the same or different. In this figure, there are two copies of Gene of Interest 1 and two copies of Gene of Interest 2. The cassettes of FIG. 17 are replaced by the cassettes of FIG. 18 by RMCE. The cell can contain additional Stable Integration Sites and integrated cassettes according the teachings contained herein.

The combination of negative and positive selection assures isolation of cells that underwent recombination in all sites. A four-site cell is useful for making bispecific antibodies, wherein two distinct heavy chain/light chain plasmids can be targeted into distinct sites.

### Example 10—Genomic Safe Harbor Sequences

Genomic Safe Harbors Sequences and the like are described herein, and many are in the literature and are publically available. Exemplary sequences are set forth below.

| Human AAVS1 sequence | |
|---|---|
| Human AAVS1 (Native RBS and guide RNA site for safe harbor insertion indicated) | |
| (SEQ ID NO: 1) | |
| GAATTCCTAACTGCCCCGGGGCAGTCTGCTATTCATCCCCTTTACGCGGTGCTACACACACT | |
| TGCTAGTATGCCGTGGGGACCCCTCCGGCCTGTAGACTCCATTTCCCAGCATTCCCCGGAGG | |
| AGGCCCTCATCTGGCGATTTCCACTGGGGGCCTCGGAGCTGCGGACTTCCCAGTGTGCATCG | |
| GGGCACAGCGACTCCTGGAAGTGGCCACTTCTGCTAATGGACTCCATTTCCCAGGCTCCCGC | |
| TACCTGCCCAGCACACCCTGGGGCATCCGTGACGTCAGCAAGCCGGGCGGGGACCGGAGATC | |
| CTTGGGGCGGTGGGGGGCCAGCGGCAGTTCCCAGGCGGCCCCCGGGGCGGGCGGGCGGGCGG | |
| GTGGTGGCGGCGGTTGGGGCTCCGGGCGCGTCGCTCGCTCGCTCGCTGGGCGGGCGGGCGGT | |
| GCGATGTCCGGAGAGGATGGCCGGCGGCTGGCCCGGGGGCGGCGGCGCGGCTGCCCGGGAGC | |
| GGCGACGGGAGCAGCTGCGGCAGTGGGGCGCGGGCGGGCGCCGAGCCTGGCCCCGGAGAGCG | |
| CCGCGCCCGCACCGTCCGCTTCGAGCGCGCCGCCGAGTTCCTGGCGGCCTGTGCGGGCGGCG | |
| ACCTGGACGAGGCGCGTCTGATGCTGCGCGCCGCCGACCCTGGCCCCGGCGCCGGAGCTCGA | |
| CCCCGCCGGCCGCCGCCCGCCCGCGCCGTGCTGGACTCCACCAACGCCGACGGTATCAGCGC | |
| CCTGCACCAGGTCAGCGCCCCCCGCGGCGTCTCCCGGGGCCAGGTCCACCCTCTGCGCCACC | |
| TGGGGCATCCTCCTTCCCCGTTGCCAGTCTCGATCCGCCCCGTCGTTACTGGCCCTGGGTTT | |
| NCACCCTATGCTGACACCCCGTTCCAGTCCCCTTACCATTCCCTTCGACCACCCCACTTCCG | |
| AATTGGAGCGCTTCAACTGGCTGGGCTAGCACTCTGTGTGACACTCTGAAGCTCTACATTCC | |
| CTTCGACCTACTCTCTTCGATTGGAGTCGCTTTAACTGGCCCTGGCTTTGGCAGCCTGTGCT | |
| GACCCATCGAGTCCTCCTTACCATCCCTCCCTCGACTTCCCCTCTTCCGATGTTGAGCCCCT | |
| CCAGCCGGTCCTGGACTTTGTCTCCTTCCCTGCCCTGCCCTCTCCTGAACCTGAGCCAGCTC | |
| CCATAGCTCAGGTCTGGTCTATCTGCCTGGCCCTGGCCATTGTCACTTTGCGCTGCCCTCCT | |
| CTCGCCCCCGAGTGCCCTTGCTGTGCCGCCGGAACTCTGCCCTCTAACGCTGCCGTGCCGTC | |
| TCTCTCCTGAGTCCGGACCACTTTGAGCTCTACTGGCTTCTGCGCGCCTCTGGCCCACTGTT | |
| TCCCCTTCCCAGGCAGGTCCTGCTTTCTCTGACCAGCATTCTCTCCCCTGGGCCTGTGCCGC | |
| TTTCTGTCTGCAGCTTGTGGCCTGGGTCACCTCTACGGCTGGCCCAAGATCCTTCCCTGCCG | |
| CCTCCTTCAGGTTCCGTCTTCCTCCACTCCCTCTTCCCCTTGCTCTCTGCTGTGTTGCTGCC | |
| CAAGGATGCTCTTTCCGGAGCACTTCCTTCTCGGCGCTGCACCACGTGATGTCCTCTGAGCG | |
| GATCCTCCCCGTGTCTGGGTCCTCTCCGGGCATCTCTCCTCCCTCACCCAACCCCATGCCGT | |
| GTTCACTCGCTGGGTTCCCTTTTCCTTCTCCTTCTGGGGCCTGTGCCATCTCTCGTTTCTTA | |
| GGATGGCCTTCTCCGACGGATGTCTCCCTTGCGTCCCGCCTCCCCTTCTTGTAGGCCTGCAT | |
| CATCACCGTTTTTCTGGACAACCCCAAAGTACCCCGTCTCCCTGGCTTAGCACCTCTCCATC | |
| CTCTTGCTTTCTTTGCCTGGACACCCCGTTCTCCTGTGGATTCGGGTCACCTCTCACTCCTT | |
| TCATTTGGGCAGCTCCCCTACCCCCCTTACCTCTCTAGTCTGTGCTAGCTCTTCCAGCCCCC | |
| TGTCATGGCATCTTCCAGGGGTCCGAGAGCTCAGCTAGTCTTCTTCCTCCAACCCGGGCCCT | |
| ATGTCCACTTCAGGACAGCATGTTTGCTGCCTCCAGGGATCCTGTGTCCCCGAGCTGGGACC | |
| ACCTTATATTCCCAGGGCCGGTTAATGTGGCTCTGGTTCTGGGTACTTTTATCTGTCCCCTC | |
| CACCCCACAGTGGGGCCACTAGGGACAGGATTGGTGACAGAAAAGCCCCCATCCTTAGGCCT | |
| CCTCCTTCCTAGTCTCCTGATATTCGTCTAACCCCCACCTCCTGTTAGGCAGATTCCTTATC | |
| TGGTGACACACCCCCATTTCCTGGAGCCATCTCTCTCCTTGCCAGAACCTCTAAGGTTTGCT | |
| TACGATGGAGCCAGAGAGGATCCTGGGAGGGAGACTTGGCAGGGGGTGGGAGGGAAGGGGGG | |
| GATGCGTGACCTGCCCGGTTCTCAGTGGCCACCCTGCGCTACCCTCTCCCAGAACCTGAGCT | |
| GCTCTGACGCGGCTGTCTGGTGCGTTTCACTGATCCTGGTGCTGCAGCTTCCTTACACTTCC | |
| CAAGAGGAGAAGCAGTTTGGAAAAACAAAATCAGAATAAGTTGGTCCTGAGTTCTAACTTTG | |
| GCTCTTCACCTTTCTAGNCCCCAATTTATATTGTTCCTCCGTGCGTCAGTTTTACCTGTGAG | |
| ATAAGGCCAGTAGCCACCCCCGTCCTGGCAGGGCTGTGGTGAGGAGGGGGGTGTCCGTGTGG | |
| AAAACTCCCTTTGTGAGAATGGTGCGTCCTAGGTGTTCACCAGGTCGTGGCCGCCTCTACTC | |
| CCTTTCTCTTTCTCCATCCATCCTTCTTTCCTTAAAGAGCCCCCAGTGCTATCTGGACATAT | |
| TCCTCCGCCCAGAGCAGGGTCCGCTTCCCTAAGGCCCTGCTCTGGGCTTCTGGGTTTGAGTC | |
| CTTGCAAGCCCAGGAGAGCGCTAGCTTCCCTGTCCCCCTTCCTCGTCCACCATCTCATGCCC | |
| TGGCTCTCCTGCCCCTTCCTACAGGGGTTCCTGGCTCTGCTCTTCAGACTGAGCCCCGTTCC | |
| CCTGCATCCCCGTTCCCCTGCATCCCCCTTCCCCTGCATCCCCCAGAGCCCCAGGCCACCTA | |
| CTTGGCCTGGAACCCCACGAGAGGCCACCCCAGCCCTGTCTACCAGGCTGACCTTTTGGGTG | |
| ATTCTCCTCCAACTGTGGGGTGACTGCTTGGGCAAACTCACTCTTCGGGGTATCCCAGGAGG | |
| CCTGGAGCATTGGGGTGGGCTGGGGTTCAGAGAGGAGGGATTCCCTCCAGGTTACGTGGCCA | |
| AGAAGCAGGGGAGCTGGGTTTGGGTCAGGCTGGGTGTGGGGTGACCAGCTTATGCTGTTTGC | |
| CCAGGACAGCCTAGTTTTAGCGCTGAAACCCTCAGTCCTAGGAAAACAGGGATGGTTGGTCA | |
| CTGTCTCTGGGTGACTCTTGATTCCCGGCCAGTTTCTCCACCTGGGGCTGTGTTTCTCGTCC | |
| TGCATCCTTCTCCAGGCAGGTCCCCAAGCATCGCCCCCCTGGCTGTTCCCAAGTTCTTAGGT | |
| ACCCCACGTGGGTTTATGAACCACTTGGTGAGGCTGGTACCCTGCCCCCATTCCTGCACCCC | |
| AATTGCCTTAGTGGCTAGGGGGTTGGGGGCTAGAGTAGGAGGGGCTGGAGCCAGGATTCTTA | |
| GGGCTGAACAGAGCCGAGCTGGGGGCCTGGGCTCCTGGGTTTGAGAGAGGAGGGGCTGGGGC | |
| CTGGACTCCTGGGTCCGAGGGAGGAGGGGCTGGGGCCTGGACTCCTGGGTCTGAGGGTGGAG | |
| GGACTGGGGGCCTGGACTCCTGGGTCCGAGGGAGGAGGGGCTGGGGCCTGGACTCGTGGGTC | |
| TGAGGGAGGAGGGGTCGGGGGCCTGGACTTCTGGGTCTTAGGGAGGCGGGGCTGGGCCTGGA | |
| CCCCTGGGTCTGAATGGGGAGAGGCTGGGGGCCTGGACTCCTTCATCTGAGGGCGGAAGGGC | |
| TGGGGCCTGGCCTCCTGGGTTGAATGGGGAGGGGTTGGGCCTGGACTCTGGAGTCCCTGGTG | |
| CCCAGGCCTCAGGCATCTTTCACAGGGATGCCTGTAC | |
| CHO AAVS1-Like Region Sequence | |
| (Guides for Insertion are shown further below in Example 13) | |
| (SEQ ID NO: 2) | |
| CCAGCACCCACATGGTGGCTCACAACTGTCCGTAACTCCAGTTCCAGAGGATCTGATGCCCTCTTCTG | |
| TCTCCCGCGAGCACCTGGCACACACGTGATGCACACTTAAACACATGCAAGCAAACCATCAGACACAT | |
| AACTTTTTTTTCCAATTTTTTAAAGATTTAGTTATTATTATTTACTTAATAAATATTTATTATATTTA | |
| TTACATATACAGTTTCTGCCTACATGCCAGCAGAGGGCACCAGATTGAATTGTAGATGGTTGTGAGCC | |
| ACCATGTGGTTGCTGGGAATTGAACTCAGGACCCCTGGAAGAGCAGTCAGTGCTCTTAACCTCTGAGC | |
| CATCTCTCCAGCCCCTCCATTTTTTTTTTTTTAAATAAAGAAATGTAATGTCCTAAGTGGGGCTTAGA | |
| GAGTGGAAGCAGATAAAGAAAGATGGAGTTAAGAATTTTAAGAAGCCAGTTGGCGGTTGTGCATGCCA | |
| GCACTCAGGAGGCAGAGGCAGGTGGATGGATCTCTATGAGTTCGAGGCCAGCCTGGTCTACAGAGAGA | |
| GAGTTCCAGGACAGACTTCTCCAAAGCTACAGAGAAACCCTGTCTGAACCCACCACGACCACCACAAA | |
| GAAAAAAAGGATTTCAAGAGGAGAGCCAGGTTTATAGCAAGAGAGAAAGTTGTGAACTAATGCCCAGG | |
| GCTTAGTGTGGCCTACCTCTGGGCTGGGTCTCTCTCTGAACACAGGGTGGAGCTGCCCCGGGAGGAAG | |
| AAGCGGCTCCGTACAGTCCCGAATTCTACAGTGGCTGGGAGCCTCCCGCCACTGACCCGCAGGGCCGC | |
| GCCTGGGAGGACCCGGTGGAAAAACAGCTACAGCATGAGAAGAGGCGCAGGCAGGTGAGGCAGGGTTG | |
| CCGGGGGAGCACTGGGCTCCCCGTTTCTGCACAACATGGGCGAGCAGGACGTCTGAGGTCTAGCCTGC | |
| CTGACCCCAAGCTCTCTCTCTTCCCGCAGCAAAGCGCCCCCCAGATCGCTGTCAATGGGTGAGTGACC | |
| GCTGCAGGGTGGCCAGGGATGGGGTTGGGAGGACTGAGTCCCGGGGTCACCCCGGCTCTGACTCCGAC | |
| CCTCCCCCTTTTTTCTTGTCTTTTTTTTTTTTTTTTTTTTTTTTTAAACCTCTGCCTTCCCGGCTCTT | |
| TGCAGGTGGGTGAGGTGGTGAGGAGGCGGGGCTGGGGTGGGGGTGGGGGAGGAGCCAGGAGGGAGGGG | |
| GGGAGGAGCCCAGAACTCTGGGTCCAAGGGAAGAGGGAAAGGAGGCTTAGTTTGCTGAAGCTATGAGA | |
| GTTAGGGGCTGAAAGTGGGTGGGTCTAAAGGCTTGGACCCCACACCCCCACCCCCGGCATCCTCAAAA | |
| GATTGAAAAGGTGCAGTTTGGTGTTCTAGGACCTGGGAGAGCACCATGCTTGAGTCCCCAGAGCACAG | |
| AGCACTGGGTGTCAGAGAAAAAAAAAAAATGGAGACCAAAAAGCAGGGTTGGGACTTCCGAGGATTCA | |
| GGGACAAGTTTGAGGAAACGTGAGAAAGTGCTGGCATCCCTGGACCACTAACTGAGGTGGGACTTCCG | |
| GCTTCCTAATGCGCAAAGGAATAGCACGTACTGAGCAAACTGGAATGCTCCCAGGGCTGAAAGAATGG | |
| AGGAAATTGAAGGTCAAGGCACGGACTCCTGCCTAGGTCCCTGGGAAGGAAAGAACTAGGGACCTAAA | |
| TTTACAGTTCTACCAAACTATGGAAGCTGAGGGCTGCAGGTCCAGGTGAGGAAGTGATGGAGAGGGGG | |
| TCACAGCCCTAGGATCCTTGGGGAAATAGGGGCCAGGAGTGGAGGGCGTGGATGTGGCTTGAGAACAA | |
| AATGATAGACTTGGAGGAGAGGAATTGGGGGCCTAGGTGAGAGCCCCAGCAGAGGGTCTCAGCAGGGA | |
| CGGCATACTGGGAGCTGTCAGTCCCACACATGGGGCGCCGAGGCCCTGAAGAGTCCCCTCCTCCCTTC | |
| CACAGGTAGGCCTGATCCGGGATGAGGTCTCTCTTGCTGGGGGCGCCAGAGCTAATCGTCCCCCAGGC | |
| TGCCTGGTGCTGCAGGGCCCTCTTGTCTGTCTGTCTGCTTCTGAATCTTGGGCTCAGCACCTGCAAGC | |
| TGTTTACTCGCCTTCTCTGGCTGTAATTTCTTTGCCTGGAAGGGTGAGGACTCTCTGGCGCTGTAAGG | |
| GGCTTGCAAAGAGCTCAGTGCCGTGACTCAGCCTGAGTTCAAATCCAGCTGCATGAAGAACAGTACAG | |
| AGTGACCCTGACAAGGGCAGCCTAGGGCCAGCTCAGTCACACCTTTCTCTTTCTTGTGCACTGGCCGT | |
| TACTACAGTATCCCTCGGTTCCTTCATATAGAAAGAGAAATAGTGAGCCGGGCAGTGGTGGCGCACAC | |
| CTTTAATCCCAGCACTTGGGAGGCAAAGGCAGGTGGACCTCTGTGAGTTCAAGACCAGCCTGGTCTAC | |
| AAGAGCTAGTTCCAGGATAGTCTCCAAAGCCACAGAGAAACCCTGTCTCGAAAAACCAAAAAAGAAAA | |
| AAGAAAGAAAGAGAAATAGTGAGACCGGCAGTGGTGGTGCACGTCTTTAGTCCCAGCACTGGGGAGGC | |
| AGAGGCAGCCGGATTTCTGTGAGTTCAAGGATAGACTGGTCTACAGAGTGAGTTCCAAGACAGCCAGA | |
| ACTAAACAGTGAAACCCTGTCTTGGAAAAAAAAAAAAGTGAAATAATGGCCATATTCTGGTGATGGTG | |
| TAGGCCTGTGGTCCCAGCTACTCAGAGACATGAAGCAGGAGAATAAAAATCAAGGCCTGCTTTGACTA | |
| CAAAGTGAGCTTCAAAGGCCAGCCTGGGCAAAGCAACAAGGCCTTGCCTCAAAATGAAAAAATAAAAA | |
| TAAAAGAGGCTGGAGAAATGGCTTAGTGGTTAAGAGTACTGGCCGCTCTTCCAGGGGACCAGGGTTCA | |
| ATTCCCAGCACCCAGACATACAGCAGCTCACAACTCCAGTTTCAGGGAATCCGGTGTTCTCTCTGGTC | |
| TCTGTAGGCACCAGGCACTCAAGTTGTGCAGACATAAAATAACACAGAGGGCTGGGCTGGGGCTCAGT | |
| GGCAGGCATTTGCCCAGAATCCCCCAGTAAAGACATAGCTCAGTGAATCCAGAGCTGAGGGGCTGGGC | |
| GTATATTAATGGTGGAATCCTTGCCTAGAATTCAACCAGCGAAGGGCTGTGGCCGTGGCTCGGCTGTA | |
| GAACCCTGTCCTGGTATCTACCATGAAGGGCTGGGACATGGCTCAGAGATAAAACACTTGCCTAGACT | |
| CTACCGCTGAGAGCCTGGGGTGTGGATCAGTGGACAGTGCCCGCCTAGCATGCACAAGGCCCCTGGGT | |
| TCAATCCCCTGTACCACAAAAAAAAGGGGGGGTGGAGGGAGGGTAAGAGTGAGATCTCAGGAGAAGGA | |
| AGGAACCAAATTCATGGAACTACAAGGGAACTCCAGGAGAATCGAAGCGTTTCTGGCGTACGTTGCTG | |
| TGTAAGCACAAGGGTCGGCTATTTTTGCACCCTGTTCATTATCCTAGCGGGTGATGGGAATAGATCTG | |
| CTGTCTCTAGCCGATTCCTCATGATCCTCACTGATGAAAATGCAGGTGAGGGGCTGGAGAGATTAAGA | |
| ACACTGTCTGCTCTGGCACTGGACCTAGGTTCATTCAGCTCCCCACAGCACATGGTGGCCCACAAATA | |
| TCTGTAACTCCAGCTCTAAGAACCCAGGTCTAGGACACCCTCTCCTGGACTCTGTGGCTACTGCACAC | |
| AGGTGATGCACATACACACACATGCATGCAGGCAACACACACACACACACACACACACACACACACAC | |
| ACACACAATGCATGTGAACGACTGGGGATGAAGCTCGGAAGCTAAGCACTTCCCTGGCATGCACGGGC | |
| CCTGGGTTCAATCCCCAGCACCCCATAATGAATTAAATCGTTATCATGATACGGTGTGTTTACTGCAT | |
| GGTGCCAGGCAAGGAAATGAGCTAACTCCATTCAAGCTGTGACTCCAGTGTCAAGCCTGTATTAACAT | |
| ATTAACCTGGGCCTCTGCTCTGACCCCCTGCTTGGCTCTAACCCCACCTCACACCTTAGAGTCCAGAC | |
| CAGCAGGGCTGGCTACCTCCTAATCTCCTGCTGGTTTCTTTCTCCCCAGTCATCAAGATCCAGACCTG | |
| GAAGCCGCCGAGCTAGAAGAGAGAGCCAGAAAGTGGGTTCTGTGTAACTATGACTTCCAGGCCCGAAA | |
| TAGCAGCGAGCTGTCTGTCAAGCACGGAGATGTGTTGGAGGTTAGCGGTGTGGGGGGCCTGAGACCCT | |
| GAAATTGGTCAATTTAGCCCTAGGTATAGAACCGGAGCGTGAATTCTCTCCTTATACGCCACCTAGGT | |
| CCTGGATGACAGGCGCAAGTGGTGGAAGGTTCGGGACCATCAGGGACAGGAGGGTTATGTACCCTATA | |
| ACATCCTGACACCCCACCCTGGACCTCAGGTGCACCGCAGCCAAAGTCCTGCAGGAAACCTAGTAAGT | |
| CGGCGTGTTCTTGCTTCTTCGGGGAGAAAGGGGGGCAAGATCCTAGGTCCTGGGGATGAGGACAGAGA | |
| AAATCAGGTGTGAAGGTTGCTGTTTGGAAAGGGGGGGGGGTGGTCAGATGTTTATTGGGAAAGGAGCT | |
| GGAAGCCTCTCTTCATTCCCTTCCAGGAGACGAGTACTCCTCCTCCCCCACCCGCACCAGCTCCAGCC | |
| CCTGCTCAGGTGCGACCCCACTGGGACAGTTGCGACAGTCTCAACAATTTGGACCCCAGCGAGAAGGG | |
| TGAGTGGTGGAGCGTCACTCTGGGAAGTGATCCTTGTCTTCGCTTTTCAGGCTCCACCCTGGGCACCC | |
| TAGCGGCTCCCAGCCCCCTGACCCCAGAACCCCTGAGCGCGCACTCCCCTCCGCCCCCCCCCCTCACG | |
| GTTTCGCTTCTGCAGAGAAATTCTCCCAGATGCTCAGTGTCAATGAGGAGCTGCAGGCGCGCCTTGCG | |
| CAGGGCCGTTCGGGTCCCAGCCGGGTAGCCCCGGGACCCCGCGCCCCGGAGCCTCAGCTCAGCCCGCG | |
| CTCTGAGGCCTCGGTGGTCCGTGCCTGGCTGCAGACCAAGGGCTTTAGCTCGGGGTGAGTGGGGCTCC | |
| CCCCGGGGCTAGTCTGAAGAGACCTGTGCTTGAACTGAAAGGCGAGGTTCCCATTGGTCCAGGGGTGG | |
| GGGCGTGGAAACTGTGGAGCAGGCCCAAATTGCAACGCCCAATGCCCAGGGACAGGCTCCAAACGGAG | |
| GCCACAGGAAAGGAAGTCCCATCCCCTTTCCGAAGCCCCAAATCTCCAAGAGTTTGAACATCCCCCCC | |
| TCCCCCCAGCTTCCTTGTTTGAGAACTCTGATTGCACAAGCAGCTAGGTAGGTGTGGCGTGATTGGTG | |
| GAGGGCCGAGGGAGCTTGATGAGCTGTGATGGCCCCTGCTGCCTCGCTCAGGACTGTGGACGCGCTCG | |
| GCGTGCTGACCGGAGCACAGCTCTTCTCGCTGCAAAAGGAAGAGTTGCGGGCGGTGTGCCCCGAGGAA | |
| GGGGCGCGGGTGTACAGCCAAGTCACCGTGCAGCGCGCGCTGCTGGAGGTGAGCGAATCCTTGGGGCC | |
| GGACAAGGCGACGGAGGGTAGGGTGGGGATGGGGGACCTGGGGGGAGGGGGTCGTCCAGGGTTCACAT | |
| ACTAAGATCTTGATTTCTACCCCGCTCTGCAGGACAGAGAAAAAGTGTCGGAGCTGGAGCCGTGATGG | |
| AGAAGCAAAAGAAAAAAGTGGAAGGCGAGACCAAAACAGAAGTTATTTGATCCTTCCTGACTCGGTCA | |
| CAAAACGTGATGGCATGGCGGGGCTCCCAGCGCCCCCTAGGACAACAGTCGCCAGACTCCTCCCCGTG | |
| ACCGGGGACAGTAGATGTCCCGAAGGATCGCCCACCCTCATCTCCCGGCTCACTCGCTCGCTCGCTCT | |
| CCTGGCGGGCAGGCTGCGCTGACAGTGCCGGCTGGAATCCTTCCGGGGGACCTCAGACTGACGGGGAC | |
| GGGGACGGGGACGGGGACGGGGACGGAGCATACAGACACTACCAGAGAGGCACGCCCAAGAGGCGCAC | |
| GGAGGGAGGGCCCTGGGCGTCGTGACGTGCTATAAACAGCCTCCTTTCTAGACCATGCGTGTCACCTG | |
| CTGTCCCCTTCTCTCGCCGGCTACCCAGGAGCCAGGAATCTGAGAGATGCCCCACGCTTCCTCCCCAT | |
| AAACCTGGAGAGTCCAGCCCAGGCTTCCTAATCACCAGTCTATCCTCGCACTGGCCCCATCTACATCC | |
| CTTCTCCTGTTCAAAACCCTCGCCTGGCTGGCTCCTCGTTGTTCTCAGTCCTGTCTCCTGGTGTTTAA | |
| GGCCTGGGCTTTTCTCATTGTCTCCGCCCACCCTGCATTTCGGCCCAGCCGCTCCAGACCACAAGCGG | |
| TTTGCACTTAACGCTTCTGAGGGTTGGAGCGGCCCCCATCACCCTGGCTCGGCTCTCCTAGCCACACC | |
| GTGGACACCCGTGTCCAGCCTCTAAGGACCGGCCATGCAGATCTGGACGCTCCCGGGGCATGCCACGG | |
| GCTCTTGGTTCTTCCTGGCCCCTCAACAACTTTCTCCCTGCCAAGCCCTGCAACTTGTCCAGGTTATG | |
| CAGGTGGATGGTAAGAGCCGGTTTTCTCATCCGCGCTAGGTTTATCTAAGGCCTTTCTTTTCCCTGCA | |
| TCCTTGGAACACTCCCAAGAGTCCCACCGTTGCAGTCGGCCTCTGCTCCCCGCGCAGCTCAGTCCTTA | |
| CCTGGGCCACCAGGTGGCGCACCTCGAATCTGACCCAGGAGGGCCAGCCTTGGGCTGACTTCACTAAG | |
| CCCCCTTTCCTTCTGGAACACTGTAGCGTTCCAGTAAGCCTTTAGTGTCCATTCCCTTGGTTTCTCCT | |
| GGTACATGAGATAAAACCTAACTCCAGCATGACAGCCGATGGCCTGTGACCCCTATGGGCTCAGGTCG | |
| CCCTTCCTCTCTGTTCGGGACTCCAGGCACTGGTCCATGCTGTTGGTTCTGTTGGGATGTCTTGGCTC | |
| CATGGTGTCTTATCACTGCCTGGGGCGTCATTTCTTATGTCGCGCTTGGTTGGTTTGTTGGAGGCCGT | |
| CTGGGTACAGCCCCAAACTCTCGGTCCTCCAGTTTCAGTTTCCTGCATGTGGGGATATTGGCAGGCGC | |
| CCTGCTGCCACCCTCTTTTCTAATCGAGAAACCAAAAGTACAAGCAGTTGCCCAAGCTGTTTTGATTC | |
| CGGCAGTGAGGTCCCAGACTACAGACTGAAATGCCAGCAGGAGCCATCTGGCTTGCTGGGACATCAGG | |
| TGATCAGGTGCCTGTGGCTGGCTCTCTGTGGTTTGGAGTCTGACCTTTTCATCCTGACTTGACCCTCT | |
| GTCGATCACTTTGTCCATCCATCACTCCCCAAGTCTACATCCAGCCAGGGGCACCTGTCAGAGCTCAA | |
| GCCGGATGGTAACCTGGTGGTCAGGCCTCCCAGCTCAGGTGGAGCTCAAGTTCTTAACAGAGCCATGA | |
| TCACACACAAAGCCATCACCTCAGCGCCACAGCACGCCAGGCCTGCTCTACCCCACGCTGCACACGGT | |
| TCTCATCATCATGCAAAAGGTGCTTCCTTCAGATACAGGGCTCACCGTCACCTTCTAGCATCTGTCTG | |
| TGCAGCTTGTCATGGGGCCTACTTTTGACTGTCATAAACACCACACACGCACATATATATACACACCA | |
| GATACACACACACCACACACATGCCCAATACACTGTGCATGCGCACACACAAACACACACACATACCT | |
| CATACACCATACACCCTATAACCCACACCAGCCATACCACACACCACATATACACAGTTCACCTCAGA | |
| CAGCATGGCACACCACACACACACACACACACACACACACGCGCGCGCGCGCACACACACACACACAC | |
| ACACACACACACTCCGCACTCTCCCCTTCTCCACAGCACTGTAGCTGAAATCCACACAGTGGCAACCT | |
| TCCTCAGTGTACTGGCTGCTGGACCAAGCTGTTCACTCCTGTGACGCCAGCTGGCAGAACAGCCCATT | |
| CCTGACTGTCAGGATGGAGGAGGCACCACGCGATCCATCTCAAGACTGATTCCTGGCTCTGCCCCAGT | |
| CACTGTGGCCACGAAGGACTACTTACCATCACCTACTCCTTTCCCAGAAAACCTAGACTTGCGGTTTC | |
| CTATGTTGGCCATCCTACCTTTTCAATGTTAAGCCACTGACTCCGCTCACTTCCAAAGCACTGAGGGT | |
| CAATGTGAGCACCCGGATCAGGTCACAGGCTTCCTTCTGACCCCCCCTACCTCACCTGGGGCTCTTTC | |
| TCTCCAGCTGCTCACTCGAGCAAGCTCCCCTCCCCACACCTGTGAGCAAGCTCCCAGCCACCCACTGG | |
| CCCTCATCCAAATGGATGAGCGGTTTCAGTCAGATACACAGGCTGAGTATACAAGCAGGAACCAGTGC | |
| CCCACACCCAGGGGGAGACAAGTCACTGAGTGGCAATGTCACGACTTTATTTGTGGTGCCTGTGCTTT | |
| GTCTCAAAAATACCTTCTCCCCCTCCCCAGACAATGGGTGGGAAGGAGGCAGCAAAAATAGAAGACAA | |
| CCCTCCCTATTGCACACGGACCCTATATACAGGCCCACCTGGCAGAGGCCAGTGGGGCTCTTGGCACA | |
| TTCCTGGATCCCTGCTGGGGAGGGAAGGGATACTGGGTAGCATCACACGTGAGGTGGGCCCGGGGCAG | |
| CCACTCTGCTCCTGGATACTGATCCTGGCTTCCTTGGTCCTTGCTTCCTTCCTGGTCCCATCTCTGGT | |
| GCCTGCCCACTCTCGGCAACATTTCCCTACCTGGCTCAGCCTCCCACCTCCACCCTGGTTCTGGGGAC | |
| TCTGTGCTTTCCTCCGGGTTCTGAGGTCCCGAGAGGAGGTTATGGCTTCTCAACAACTTCCCCCGGAG | |
| CCCTGTCACTCATGTTCACTCGGGGGAAGGGGTGCGTGTGTCAAAAGCAGCTGTATAAATACGGTGCG | |
| GGAGCCCCTCCAGAGTCACTTGGAGAGCTTGCTAATGACGCGGATCAGTGCTGCATTCTCATCCTTGA | |
| GCCGCTGGTTGTCAGCGCGGAGGTCGGACAGGGCCTAGGGGGCAGGGTGGAGTCAGCTGGGCAGGGCG | |
| GGGCAGGGTGGGCTCTGGCCACCGCCCTTCACAAGCTCGTTACCTTCAGCTCCTCCTCCAGCTCTGCG | |
| GCCTTGCGCTCCAGGGCCCTGCGCTCCTGCAGGAATGGGCTGGGCTCAGAAGCAGGGTAAGGGCAGGG | |
| GACAGGGCAAGGGCGGGACACCACCCCAGCGGCCCAAACTCACGAATCTCTCCAGCTCCAGGAGGGCG | |
| GGCCTCTCGGCAAAGCGTTCCTGCCGCTGGTAAGGGCAGAGAAGACTGGGCGTCAGGAGCTGCTTCTT | |
| ACCCCTAGGACATCAGAGCCCTGCCCCCCCCCCCCGAGTGGGGGACCTCCAACCTCCCAGCCACGGCC | |
| AGGCCCCTTGCCACTGGGGCTCTGACTCCCACTGCCCCAACAGCTGGTTCTTAGGTCTCAGTATCTGC | |
| ACCTGCGTGGCCCGCTCAAGCTCCACCTTGAGCTGTGCCAGCCGCAGGGTGGTCTCTGTCAGGGCCTC | |
| ACGAAGCCGCTCGTTCTCCCTCCGAAGCTCCATGTACAGCTAGGGACACAGAGGAAGCAGGCAGGCTC | |
| AGAAGGGCCCGGGAAGGGGCCAGGACAGGGTGGGGTGGGGCAGGAGGTAGCATGCGGCACCTTCCGGA | |
| AGCTTCCATCGGGTTCTTCCTGTTCCTGCTTGGATTCTGGATTGAGGTCTCTCTGCAAACGCTGTCTA | |
| CGGGCAGTGGAGCCGCCATCCACGGTGCTGGACAGAAATTCAGGCCTTAGGGCCCAGGCCCTGCCCGA | |
| GGGGTGCCCCAGCCCCCACGCATGACCCGGCCTACCTGCACTCCAGGCTCCGTTCTGCCGGCCCCGCC | |
| TCCTCCCCCTGCAGAAGAGCCCTGAGAGTTCAGTCTCCATGCAACGTCCTCCCTCCAGCCCGCCCGGC | |
| CTCCACACAGCATCCTCACCTCCGCGGCCCCTCCCTCCAGCCCGCCCGGCCTCCACACAGCATCCTCA | |
| CCTCCGCGGGCCCCTCCCTCCAGCCCGCCCGGCCTCCACACAGCATCCTCACCTCCGCGGCCCCTCCC | |
| TCCAGCCCGCCCGGCCTCCACACAGCATCCTCACCTCCGCGGCCCCTCCCTCCAGCCCGCCCGGCCTC | |
| CACACAGCATCCTCACCTCCGCGGCCCCCTCCCTCCAGCCCGCCCGGCCTCCACACAGCATCCTCACC | |
| CTCCGCTGCCCCTCCCTCCAGCCCGCCCCCGCCTCCACACAGCATCCTCACCTCCGCGGCCCTCCCTC | |
| CAGCCCGCCCGGCCTCCACACAGCATCCTCACCTCCGCGGGCCCTCCCTCCAGCCCGCCCGGCCTCCA | |
| CACAGCAGCATCCTCACCTCCGCGGGCCCTCCCTCCAGCCCGCCCGGCCTCCACACAGCATCCTCACC | |
| TCCGCGGCCCCTCCCTCCAGCCCGCCCGGCCTCCACACAGCATCCTCACCTCCGCGGCCCCTCCCTCC | |
| AGCCCGCCCCCGCCTCCACACAGCATCCTCACCTCCGCGGCCCCTCCCTCCAGCCCGCCCGGCCTCCA | |
| CACAGCATCCTCACCTCCGCGGCCCCTCCCTCCAGCCCGCCCCGCCTCCACACAGCATCCTGACCTCC | |
| GCGGCCCTCCCTCCAGCCCGCCCGGCCTCCACACAGCATCCTCACCTCCGCGGGCCCTCCCTCCAGCC | |
| CCCCCCCCCCCGCCTCCACACAGCAGCATCCTCACCTCCGCGGCCCCTCCCTCCAGCCCGCCCCCGGC | |
| CTCCACACAGCAGCATCCTCACCTCCGCGGCCCCCTCCCTCCAGCTCCGCCCGGCCTCCACACAGCAT | |
| CCTCACCTCCTCGGCCCCTCCCTCCAGCCGCCCCCCCCCCGCCTCCACACAGCATCCTCACCTCCGCG | |
| GCCCTCCCTCCAGCCCGCCCGGCCTCCACACAGCAGCATCCTCACCTCCGCGGGCCTCCCTCCAGCCC | |
| GCCCGGCCTCCACACAGCATCCTCACCTCCGCGGCCCCTCCCTCCAGCCCGCCCCGGCCCTCCACACA | |
| GCATCCTCACCTCCGCGGCCCCTCCCTCCCCGCCCGGCCTCCACACAGCATCCTCACCTCCGCGGGCC | |
| CTCCCTCCAGCCCGCCCGGCCTCCACACAGCATCCTCACCTCCGCGGCCCCTCCCTCCAGCCCGGCCC | |
| CCCCCCCCCCACCCCCCGCCTCCACACAGCATCCTCACCTCCGCGGGCCCTCCCTCCAGCCCGCCCGC | |
| CTCCACACAGCATCCTCACCTCCGCGGGCCCTCCCTCCAGCCCGCCCCGCCTCCACACAGCAGCATCT | |
| CACCTCCGCGGCCCCTCCCTCCAGCCCGCCCCGGCCTCCACACAGCAGCATCCTCACCTCCGCGGCCC | |
| CTCCCTCCAGCCCGCCCCGCCTCCACACAGCAGCATCCTCACCTCTGCGGCCCCTCCCTCCAGCCCGC | |
| CCGGCCTCCACACAGCATCCTCACCTCCGCGGCCCCTCCCTCCAGCCCGCCCCGGCCTCCACACAGCA | |
| TCCTCACCTCTGCGGCCCCTCCCTCCAGCCCGCCCGGCCTCCACACAGCATCCTCACCTCCGCGGGCC | |
| CCCTTCGCTCGTGGCCGACCTTTCGATGCTCCCTGGCCGCCTGTGGTCCCTGGCCCTGCCCGTCGGGC | |
| GCCTCTGCTGGGGAACCAGTGGGAATCAGCTCAGACACCACCATAGGGGCCCCTGTCTACTGTGCAGG | |
| GAACCTGACTTAGCCCCCAGTGAACAAAGACACTTTATGGGGAGACAGGATGGCTCCCTGGGGAGCGA | |
| CTTCCCAGAAAGCCGACCTCACCTCTCTGGGCAGGGCCCTCAGCGTTCTCCACGCCAGGGACCCGAGG | |
| TCTCCGAGAAGGGTCCTGCCGGGGAGGAGCACAGTCAGAAACAGGGAGACGGGTCCACCCGCCCCAGT | |
| TCACCTGACCCTGCTCACCAGGCTGGGCAGGGCAGGCTGCTCTGGCTCTGGAGCCTTCCCTGCCACCT | |
| TCTCTGCTTCCTTCAAGTCTGTCAAGGTCACGCCCTAGTGTAGAGAACTCGGTCAAGGAAAAAGGGCC | |
| TAGACTTCCACAGGACTCAAGTTCAAGACCCCGGCCCTCCTCCCTCAGACCCGGGAGCACAGCCCCAG | |
| CCCCATCCCACACCTGTGTAGACCTCCGAGACTGGCGCATAAGGCGGGAGCGAGCCTTCCGCTGAGAC | |
| TCGGACTCTTCATCACGCACAGGCATCTGGTAGGACCTGAGTGGAGAATGTCCCCTTGGGTGCTGTCG | |
| CACTGTGAATGACACCTTAGGGGAGTGCACATTCTGGCAGAGAACGTGTCAACTGGGCAAACAGGACC | |
| CCAGGAGCCTACCCAGAGCCCCAGAGACCCCTAAACACTGTCTTCCCCTAGCCTCTTTACCTCCGCCG | |
| ATCCCTGGAGTCAGGTAGGGCTGCTGCAGAGGCTGCGAGGGCATTTGGCTTCACTGGGGATCCAGGCT | |
| CCCTCCTGGAGGATGGGGCGGAGTGATCCGAAGAAGGAGGCATGGCAGCCTCACACCTGTATGGATTC | |
| ATTCATTCATCAGCAAATATTCCTCAAGCCCGCATTCTGTGTCAGGCATAGGAGAGACCACAGAGAAG | |
| GAGCCAATCATGGCTGCTGATGAGCCATTTCTGGGCAAAACAGATAAAACAAACAGCAGCCAAAGAGA | |
| CCAGTGTGGAGCTTGGGGAGAAAAGGTGCTTGGAAAAAATAAAGAGAATAAGCAATTATTTGATGCAC | |
| CCTAAGGGCTTTCTCAGATCTCAAATGCCAGGATGGCACCAGACCTGTCCCCTTGCCCCAGCCACTGG | |
| TACTTACAGGGTAGAGGGCTCTGGCACTTTCTGGGCAGGGGTAGGGGTTATTCTGGCAAGACGGGGTT | |
| CCCTGGCCTGTGGAGACAGGAGAGAAGCAAAGGAGGCACTGTCTGCCCCAAGGCAGGAGCCTGTACCC | |
| CACACACTTCACGGCACCTACCTGAGAGGAGGCCTTTTCTAGGAGGGAGGAGGAGGCTGAGCGCTGCA | |
| GACCGAGAACCCCCTCTGCACCCCTCCTCTCTGAGGGACCCAGGGCACCAGAGCTTCCTGTCTTCTGG | |
| AGACCGCCGCGCCTGGAGAAGGGAGCCTCTTCTGGCGGCTGGGAGAGGAAGAAGGTCTTCATTACTGA | |
| GCAAAGCAATGACCCTTCTCCTCAGAGCCTACGCGTGTAACTCCAGGGGAATTACAGTAAACCACAGC | |
| CAAAGCAATGACCCTCCTCCTCAGAGCCTACGCGTGTAACTCCAGGAGAATCACAGTAAACCACAGCC | |
| AAAGCAATGGCCCTTCTCCTCAGAGCCTACGCGTGTAGCTCCAGGGGAATCACAGTAAACCACAGCCA | |
| AAGCAATGACCCTTCTCCTCAGAGCCTACGCGTGTAGCTCCAGGGGAATCACAGTAAACCACAGCCAA | |
| AGCAATGGCCCTTCTCCTCAGAGCCTACGCGTGTAGCTCCAGGGGAATTACAGTAAACCACAGCCAAA | |
| GCAATGACCCTTCTCCTCAGAGCCTACGCGTGTAACTCCAGGAGAATCACAGTAAACCACAGCCAAAG | |
| CAATGGCCCTTCTCCTCAGAGCCTACGCGTGTAACTCCAGGAGAATCACAGTAAACCACAGCCAAAGC | |
| AATGGCCCTTCTCCTCAGAGCCTACGCGTGTAACTCCAGGAGAATCACAGTAAACCACAGCCAAAGCA | |
| ATGGCCCTTCTCCTCAGAGCCTACGCGTGTAGCTCCAGGGGAATTACAGTAAACCACAGCCAAAGCAA | |
| TGACCCTTCTCCTCAGAGCCTACGCGTGTAACTCCAGGAGAATCACAGTAAACCACAGCCAAAGCAAT | |
| GACCCTTCTCTTCAGAGCCTAAGAGTGTAACTCCAGGAGAATCACAGTAAACCACAGCCCAGGCAGGT | |
| GCCACCAAAAAAAAAAAAAAAAAAAACATTACTTCTTGGTCCACAAGGACCTAAGAACCAAGTCAAAA | |
| AGCCACTTTCCTCAGCGGAAGCAGAAGTATTTACCGTATCCCACCCGCTGCCCCAAACCTCACATCTG | |
| CTCAGGGCGCTCAGGCTCACCACAGGGCTCTTGGGGCTGGAGGACACAGGAGAAGACACGCCATTGAG | |
| GGCTCTTGGTTGCACAGGAGGGTGATCTGTGTGCAGGAACAGGAGAGGGGGGTCACAGGAGAGGCCGG | |
| CCGCCTCTGAGATTGGGGACCCACAAGTCCAGCTCCTTCCTCAGACCCAGGGTCCAGCATCCCTACCA | |
| GCTGCCTCTTCTTCTCCCTCATCCTCATCCCCAAGAGAGGGGCCCGCGGCCCCACCAGGCCGGCGCTC | |
| CTTCGACAGATCCTGCAGGGAGATCTTCTCACGGCTGCTCAAACGACACACGGAGCTCCTAGGAGGAC | |
| AGGGTGTCCGTGTCCAAGTCTGGGGGCGAGTCCGACCCACCCCAGGCCTAGGCATCTCTTACCTTCTG | |
| TGCTTGCTGTTGGAGGGCACCTGTGGCTCTTGGCCTCGGCTCTGAGAGGCTTCCTTTTGGTTCCGAAG | |
| CTACAAGGATGGAAGGGGGCAACTGGGGAGGGGCAGAGAGCACAAGCCCTCCAGGGTCTCCTGGCCGC | |
| CCCCTCTGTGCCACCTCTCCACCTCGAGGGCCATCACGCATAACTGGGCTAGTCACACTTTATGCAGG | |
| GTCCTGCAAACATGGGGGACTCAGTAACCCGGCAGCACACTGGCTCTGGGGCTTATTCAGGCTCTCCC | |
| AGGCTTGGCCTGGTCCAGCTGTCACTGCCTCCAGCCTCATTCCCAGGGGGATTCGTCTTCTTCCCAGG | |
| AGCGAGCACCTTGCTCAGACTTCCCCCTACCCTCCAGCACATCCAGGGCAGGACAGGGCAGGTGGCTC | |
| TTTCTGGTTATCACAGGCCAGCTCTCAGCTCAAGGACAACGGCCACCGTCCCATACTAAGCAGTCTGG | |
| TGTCGTAACCCCAGGAACACCTCTTGCCCATGCCCTCCTTGCATCCCAGTGTGCCACGGGACTCCTCT | |
| CTGGACAATGTTCCCGATGGTTCCACGAGGCCCGGGCCACCTCACTAAATAATGGAATTGCAGCCATG | |
| CCGTCTGCTTGGGGCCACACCCATGATGCCTCACTCTCCACTTTCCTAGCAAAAGTGCTAACTAGAGT | |
| GGGGGGGGGGTAGATACAGGTTCAACCTGTGTCACACACAGCTGTCTTCCCAAGCGAGCAGGCAGGAA | |
| ACTCTGGGCATAGCCTCAAGTCCTCCAGATATGGAGGTGCCTCTGTTCTTAGCCCTCCACCAGAGCTG | |
| GGCTGACAGGTGGGAATAGCGGGTCTCAGTACTGAGGGTGTCAAGGGACAAAGACTGTCAGCCCTCCC | |
| GGTTACTGTTACCTCCTCAGAGCTGCCAAGTAAAGAGGCAAACTAGAGTCGAGACTCACGTCCTCCTG | |
| TTTCTGGGCCAGTTCCTCCAAAAGGTTCATCACTTCCTCATCAGCCAGGTCACAGGGCCGCTGCCCCT | |
| GAGTAGGAGAAGGAGGCAGATGACGGTGATGGTGGTGGTGTAGTAGGGGCTCCCCCGCCACCCTGCCC | |
| CACCATCTGAGATGGCCCTTACCGCATGGGTCAGCGAATCCATGCCCCCACCGTGCTCAGCCAGGAGA | |
| CGGCAGGCGTCCTCCACACCCCAGTGGGCTGCTGCGTGCAACGGTGTCCAGCCATCTCCATCCCGGAG | |
| CTCTGTGTCGTAGCCAGCTTGGAGTAGCAGCCTAAGGGCCAGGGAGGCTTGGGTCAGATGGCAAGCTA | |
| GGCCAATGGCTGATCTCAACTTCTGTTCTGTGGCCACAGGACTACTGATCAATACCCAAGCGTTACTA | |
| GTTTTACCAGCAACCAGCCCCACCCCAAGCTCAACTGAGCCCTCCCTTGGACCAGCAGCTACTAATGA | |
| AAAAGCTCCCTCATACCACAGGGATCCCACTCCTCAGGCCCCAGGGTAAAGGGTTAGGGCAGTGGTGA | |
| GGCGATGAGGTGGATGCAGGACTCCCCACTAACGCAAGCCCATGGAGAGGATGGACCCTGAAGGGGCT | |
| GTGATGCTGGAACCACTGGAACCACGCGGTTTTAGGACACGGATCCTCAACAGTGTCAAGCAGCTCTC | |
| ACACCCTCTCTACAACTGGAGACATCACCACTAGAATCCTAACTTACGGGTACAAGCAGGAAGCACCA | |
| GTGTGTGGGAGCTGGAGAGGCTGCTCAACCCCCTCCCACGCACAGGACAGCCCTACCACAGCACGGTA | |
| AGACCCCAAACATCACAGTGCCGGAGGAGAGCGAGCCTGGCTCAGCCTTCCAGAAGGTAACAACCTGG | |
| AGCTCTCAAAACTCAGCATGGCACGAGGCGAGGCCTCTTTTGGAAGCAGTGTGATGAGGTCCTGTGTC | |
| AGTGAGGAAGGCTTCAAGCCCAGGGAGGCAGAGGTACAAGGCACAAGGTGCTGTGTGGCCCTGGGACC | |
| CTCCTCCCTCACACTTCCCAAGATTCCCCTGTCCCCTTGCAGCAGGGCACGCTGGGCTTCTTGTTACA | |
| TTCCCACATGCCAGGGTCTCTAGCCAGCTGTGCGCTCCTTCTGGTCAGTATCCTAGGAGCCTGAAGCG | |
| TGCCACCCAGCCACACCCCCTAGTCCATCAGCACTTCCTCACCTGGCAGTTTCTTCACCACCATCTCT | |
| GCCAGGGGGCCTCCCTACTGCCCACTAGTTATAGCCTCCCAAGGCCAAGGTTTTCTTTGTATAAGCTT | |
| AGTGTTATTTACCATTAGTGTGTGTGTCTGTGTGTGTGTGTGTGTGTGTGTGTGTGTGTGTGTGTCTG | |
| TGTGTGTGTGTGTGTCTGTGTGTGTCTGTGTGTGTCTATGTGTGTGTGTGTCTGTGTGTGTCTGTGTG | |
| TGTGTGTGTCTGTGTGTGTGTGTGTGTGTCTATGTGTGTGTGTGTCTGCGTGTCTGTGTTTGTGTGTG | |
| TGTGTGTGTCTGTGTCTGTGTGTTGTGCATAAATGCCAACACACATGCCCCAGTATGAAGATCATGGA | |
| TGAAGATCAGAGGACATATTCAGGATTCACTTTCTCCTTCCACCACCGGTTCCAGGACCTAACACAAG | |
| TCACCAGGCTCTTGTGTGGCCAACACTTTTACCTCTGAGCTATCTCACTGGTCTAGAAGCCAACGTTT | |
| GCAGCTGGACCCTGCTACTCCCCAGAGGACCTGTGGCAATGTCTACAGTCATCACACAACTGGGTCAG | |
| AGGTGCTGCAATGGACTGGACAGCCATCAGAATAGAATGACCCAGCCCATCAAGTCTCTCATTGGCTA | |
| CGGTGGGTACACATCTGAAACACCACGACCAGCCCAGGAGGCTAGCCCCTAACAGACACCAATATTTA | |
| CCTGTACTTCAATGAGTACAATCATAGAAGACTTTTAATACAGTCAGAAACAATAGATAACTATAAAT | |
| TCAGTGAACAGGAGTCTAAACGCAAACTCACACAAAGGGGGCCATCACAAAATTACAAAATTCAGTAT | |
| GATGGCTCACACCTGCAATCCCAGAACACAGAAGCTGAGGCAGGAGGACAGCTGTGAGTGCAAGGCCA | |
| ACCTAGGCTATCTATCCAGTACCAGGCTAGTCAGGACTACATAGCAAGACCTTGTCTCCATTAGAAAA | |
| GAAAGAAGCCAGAGGGGAGGGAGGCAAGCATGGTGGCTCTCACCTCTATCCCACAGGAAGGTGAAGGA | |
| ACAAAGAGTAGAAATTCAAGACCAGTGAACTAGAGGCGATCATGACCGACATGAGCTATTTATGGAAG | |
| AGGCCAAATAAACAAACACAAAAGTTGTCATCAGTGCATTTTTTTTTTCAGGGCTGGGACTGGAACCC | |
| AGAACGCTAGGCAAGTGCTCTATCCCTGAGGCACCCCCCCCTTCCCTCACGGGTAGACACCAGGGAAG | |
| CATCTATCTACCTATGGCCTGCGACCACAGCCCAGTGCTTCAGTTCTGGGACAAGTATTGGCTCACTT | |
| TCTCTACTAACTAGCCCCCCGGACCTATGCAGGTGACACCGGGGAAAGCATTTAAGCACAAAGACAGG | |
| AAGGAGTTCTGATCACCAGAATCCACTTAAAAACTCAGTGGATAGCTGTTATAAAAAAATGACATCAG | |
| GGTGGAGAGAGATAGATGGCTCTGCTCTTCCAGAGACCCGGGTTCAATTCCCAGCACCCACACGGCAG | |
| CTCCAGGGGTTCTGACCCCTCACACTGACATAACACAGACAGGCAAAGCACTAATTAATGCACATTAA | |
| AAAAAATAACATCATGAAATCTGCAGGCAAATGGATGGAACTGGAAAAAAAAAAAAAAAAAAAAAACA | |
| TCCTGGGTGAGGTAACCCAGCCCCAGAAAGACAAACATGGTGTGTACTCATTTACAAGTGCACATTAG | |
| CTGTTCAGTGAAGGACAATCGTGCTACAATCCACAGACCCAGAGAGGCTAGGTAACAAGGAGGGCTCC | |
| GGGGAGGGACGGTGCACGGATGCCCCAGGGAAAGGGAAAGAGAAAAGACTTTGCAGATGGAATGGGCA | |
| GGTAGGGATGGAAACAGGAGAGGTGGGGAGAGGGAGTGGAGGGGAAATACTGGGGGGGGTGGCTGCAA | |
| TGGGGGCTCACTTTGGGGGTGTTAAGGAAACCCAGCACAGTGGGAACTCCTGGACTCTGCAAGGGTGG | |
| ACCTAGCCAAGTAACGAGGGACACAGAGTCTGAACCGGCTACTTTGGGTAACAGGCAAGGCTCCCAGC | |
| AGTGGGACATCAACCCGGCCACAAAACTTTTGACCTACGATGTGCCCTGCCTGCAAGGTGTGCTGAGG | |
| TAATGGTGGCGCAGAGCTTGTGGGAGTGGCCAACCAATGACAGGTCCAGCTTGAGGTCCATGCCACAA | |
| GAGGGAGCCCACGCCTGACACAGCCTTGATGGCCAGGAGCCTGGATAGCCCGAGACCTGGGGTAGAAC | |
| CAAATACAATTGGGGGAAAAGAAAAAAAGGCAAGAAACAATTCTTAATGATATTCTGCTGTTCTCATG | |
| GATCTGTGGCTAGCCCAACTGTCGTCAGAGAGCTTTTTCCAGCAGTTGACGGGAGCAGATGCAGAGAC | |
| CCACAGCTCAGGGAACCCCACAGGAAGGATTATGGGGGGGGGGGGCGCGAGGACACCAGGAGAACAAA | |
| GCCCACAGAATCAACTAAGCAGGGCTCCTTGGGGCTCATGGAGACTGAAGGAGCTAGCCATCAGGACC | |
| TGTATGGGTCTGCGCTGGGTCCTCGCCTGGTGCTCTTGCGGGACTCCTTAACACTGGGACTGGAGCTG | |
| TCGCTGACTCTTGTGCCTGTTTGGGGACCCAGACAAGCATAACTGGTTACGCTGTGCTTGGCTGTCAT | |
| CTCTGAGATGCCTGTTCTTTTCTGAAGGGAAACAGAGGACTGGATCTGGAGGAGGGGTGGAGGGGAAC | |
| AGGGCAGAGGGGAGGGAGGAATGTAATATGAGAGGAAAAAACAACAACTACAATTATTGAGTGGACAT | |
| GGCAGCCCATCTGCAGAGACAGGCCACCCTCAGACGGAGATGGCAGCTAAACTTGCCAAAAAGGCAAG | |
| CTGAGGGATCGGCCAGAGGCCCTGCCTCAATATTAGAGTGGAGAGCAACCAGAGAAAGTACTACATGC | |
| CAACACACACACGAGTGTGAACACACACACACACACAAGTCATACCCATACACATGCACACGCGCGCG | |
| CGCACACACACACACACACCACAACCTTTAACCAGACATATAGTTGTGTGGAAACAAACCTAGTTTTC | |
| CTTGCAACTAGGACTGGCCAATGGTGAGAACTGGGTTAATGGAACACAGATATTAAATATGCACACTT | |
| CTGGAATGTTCTCCTGAAAAGGAATAGACATTCGCTCCCTTTGCCTCTGCTTCCCACCAACTTGAGAT | |
| ATAGACGCAAAGGCAGGTGAGGCAAGTCACCCTCAAGTGAGAGGCACCGCTAGAGCAGGGCGCAAGCT | |
| CTGCACTCGGAGATTTAGGGCATCCTGTCCCCCAAAAGGAATGGGCTCAGAGCGCACTGGGACTCATG | |
| CTGTAACTACAGAGACTGATGCCCCTCCCCCAGGAGCACAACTATGCAGGCAGGCTGTAAGTCTGGGG | |
| GTGGCACGAGGTCTTAAATCCTGCTGGAGAAAACCTGCCTGCAACCTTACCAGTATGAAAAGCAGAGA | |
| GGTTCATCTTAATTCAATTTGGGTCTTTGTTTTTTTGTTGTTTTTTTTTACAACAGGATCCCTCTATA | |
| AAGCACTAGCCTCACACTCAGTATATAGACAAATCTATCCTGGAATTCCAGTAATCCTCCTGCCTCTG | |
| ATTCTCAAGTGTAATTATAGACATATAACACCGTATCAAGCAAGCAAGTGCACACACGCACGCACACG | |
| CTCTTGTTACATAGCCTGGGCTAGCCTACAACTCACAGCAATCCTGCCTCGACCTCCCAAGTGAGGAA | |
| ATTAAAAGCGTATACCACCATGCCTGGCTTAATGCCATTTTTTTAGGTTGGTATTATTTTTATGCGTA | |
| TATGTTTTGCCTACATGTATGTATGCATACAAATACACACAGACACAGAGATAAATAAATGTAATTTT | |
| TAAACCTCTTTGGCTTTAGGTATGTAAACCAGGAGAAGAAAAGGACAAGAGCCCCGAAAAGCTTCCAG | |
| ACACAAAACAATCACTCTGGCCTCGCTCACCTCATCACCTCGATGTAGCCCTTGGCGGCAGCCACATG | |
| CAGGGCAGAGGCCCCGGTCCGGGGGTGGCGGGCCTCTGGCATGGCACCCCCATTCAGCCAGCACCTTG | |
| TGTCATGAAGCAGCAGTTCTTCTTCAGCCCGCTTGGCTGCCTCGACATCCACACCTGGGAGAATGAGA | |
| GGTGACAGGTGGACTCACACAGGGTGGCCTAGGAAACCCCGGCTGCGGTCTCAACTAGTCACAGCCCG | |
| GCCCCGTGACTCATCAAGTCTCTGGACCACTCAGGAGACCGGGACTGCCCCAGTGTTTCCCAACTGTG | |
| CTCCCTGAAGACCTGGGCACCACCGAGGGGGCCAAGACAGGCCAGGAATGGAAACCACAGGTCCTGAC | |
| CCCTGTGGGTCAGTATCCTCTTTATGTTTTTCTAATAGAAAACCCCACACCGGATTCCATCTAGGTTT | |
| TCCTACCCCTCCAGCTATAAGCTAAAGCCAGCGCCTTCACACAATGTCACTGCTGGTTCTTCTCCCTT | |
| TGAAGTACGATAGGCCAAACAAAACTTCACTACGGCGTTGTACGTGGTGGCTCCGGCCTCTATTCCAG | |
| ATCTCAGCCTTGGCAGGATGACCGGTGGCCTCGAATCTGAGAACAGCCTGAGCTACATACATGGTGTC | |
| AAGCCAACCAGGACTAGAGAGACAGACTCTGTCTTAGACAACAGTAAAAACTAAAACTCAAAAGCTTC | |
| TGGGCGGTGGTGCACACCATTAATCCCAGCACTCGGGAGGCAGAGGCAGGCGGATCTCTGTGAGTTCG | |
| AGACCAGCCTGGTCTCCAGAGTGAGTGCCAGGATAGGCTCCAAAGCTACACAGAGAAACCCTGTCTCG | |
| AGAAAAAAAAAAAAAAAAAAAAAAAAGCTATTTCCCAAACTATTTGCATGCATAGTTTCATTCTTGCC | |
| CAGATGTCCAGGCATTTGACACCTCGCTGGCCCACGACAGAAGTGAGAAGTGAGTGACTGCCTTGGCA | |
| CTTTGTGCTTATGCGGGTATGCTGCATGCCTGTGACCCCAACACAGGCAAGAGGCAAGAGACCAGCAG | |
| GGCTCCACAGAGACCCTGAGTCAAAAGACAAACAGAGGGGGAGGGGCTGGAGAGATGGTTTAGAGGAT | |
| GAAGTGCCAAGCCCGATGACCCAAGTTCAATCCTGGGAACTCATGAGGCAAAGGAAAGAATCAAGTTG | |
| CACAAGGGGTTTCCCTTTGTGAACCCAGCTTGGCCTCAAACTCACAGCAATCCTCAGTCTCTGGAAAG | |
| CTGAGATTAAGAGGGGGTTTTTTTGTTGTTTATTTGTTTTTTTTGGTTTTGGTTTTACGAGACAGGGT | |
| TTCTCTGTGTATCTTTGGAGCCTATCCTGGCACTCGCTCTGGAGACCAGGCTGGTCTCCAACTCACAG | |
| AGATCCACCTGCCTCTGCCTCCCGAGTGCTGGGATTAAAGGCGTGCACCACCAACACCTGGCTAAGAT | |
| TAAGAGTTACACACCACACCTGTACTCCCTGCACTCAAAGAGGCTGAGGCAGGAGGATTGCTCCAAGT | |
| CCAAGGTCAGCCTGGGCGCCAGCATGAGAGCCTGTCTCAAACACCTCAGGGGGGAAACAGAAAGCCAG | |
| GCAGACTAGCTGAGGCTGAAGCACTCCAGCCCTCACTGTGACCCTCATCCCTTAAAGCACCCCTAACT | |
| CACTGAGACCACAGCAAAATGGCCTCTGCTGAATAACTTCCTCCTGGGAAGGTTATTACTGCCCATGC | |
| TTTTGCAGTTGTGAAACTCTTGACTTGCCGAAGTTCCTCAGAGTTGAGCTGTTGTATCCAGTAGCCGG | |
| CAGCTATGTGGAACTGCCGAGCGAGCACTCGAAACTGAGACATGCTGTGAACGTCAAGTGCACTCGGG | |
| ATTTCAAAACACAGGAAAAGGTAAGGGCTCTCGTGGACAGTTGTCTCTAAACTGTTTTGTGCACACGT | |
| GCATGGCAGCGTGGTCAGGGGGCAATTCTGAGGAGCCGAGTCCTGCTTCCCACCTTGCTGAGGCTGGG | |
| TCTCTGGATTCTGCGGCTGTGCTGTGTACTCTAGGCTACCCGGCCCACAGGGCGTCCCCACAGTTCTC | |
| CCACCTTCCTCTAGGACCTGGGAGTACGGACGTGCACCAGTGCCGCCGCCGGCTTTTTACAAGGGTTC | |
| TAGAGGTGGTAACTTGGGTGCATCTAACATCTTTACTGGCTGAGCTATCTCCCCAGTTCCCCTTACTG | |
| TGTTGATTGCACCCAACGGAATACTGGGTTTGTTTTTTGTTCTGGAGCGTGCGTGAGAGAGAGAGAGA | |
| GAGAGAGAGAGAGAGAGAGAGAGAGAGAGAGAGAGAGAGAGAGAGAGAGAGATACAGACACAGAATCT | |
| TAACAGAGAGACAGAGTCTTACATTATATAGTGGAGGCTGAATATCTAGTCTTCCTGCCTCCCAAGTA | |
| CTATGGGATAGTCCTTCTGTACGCTACGAATATGGACTCTTCTCATTGGTTAATAATAAAGCTGAGTT | |
| GGCCCACAGCCAGGCAGAATAAGGGTAGGCGGGAAAGCCAAACAGAGATACAGGGAGAAAGAAGGGCA | |
| GAGTTGAGTGAGACGTAAGCAGCCACCAGGGAAGCAAGATGCCAGGTGACAGGTAAAGCCACGAGCCA | |
| TGTGGCAAAACACAGGCTAATAGAAATGGGTTGATTTAAGTTGTAAGAGCTAGTTAGTAATAAGCCTG | |
| AGCTATAAGCCGAGCATTCCGTAATCAATACGAGCTCTTGTGTATTTATTTGGGGCCTGGCGATTGGA | |
| ACTAAAGGGAAGCTTAGACTACGGACTTGCACCTCCATGCTTAGTTTATGGGTTCAGAGGACCATGCT | |
| AATGGATAAGCACTCTACCAACTAAGCTACACCCCCAGCCTATGGCTTGCAAGTTTCAAACTACATCT | |
| GTGGCTCATTCAGGATTTCCACTGGGCGTCACTGGCAAAGGCCTTCAGGTCCCACCTGGAGCGCTGGC | |
| TCAGCCATTAGAGCCATTAATGGTAGACTCACAACCTACACAAGAGACAAAAACCCACACAAGGGGTG | |
| GAATGCAGAGACTCAACCAGTTCCAAGCCAGCCGGGACTAACAAAGCAAGATCCTGGCTCATAAACCC | |
| AGGAGCAGGGTTTAGCCCAGTGGTGGTATGCCTGCCTGGAAAGGGATGGCCCCAGGTTCAGGCCTCTA | |
| CACAGAGGGCTGCTTTCCTCACCACACTCCCTCTTAACCAAGGTGAGCAGCCGCTCCCCTCAGCACAC | |
| ACATTGTACACTGCCACCATAAAGCTTTACATGGGACCCAAGAAACAGTCCTGAAAGCTGGTTCGGGA | |
| TGTTCTTTCTCATTGCAAGGCAAGGCCAACTCCATGCGGACACCGGCTGCAGCTTGGTGCTACCTGGC | |
| GGCAGCCGGGTCCTAGCTCCTTGTGTCTCCTGGCCAACTAGGGTTTCCCTTGTGGTGGCAGAGTTCAA | |
| GAATGCATGGCGAAAGTCCACCCGCAGCACAGTCACAGGGAACAGGGCAGGGAGGGCCAGGCCCGCCC | |
| TCGTCCTCCAGACTCCTGCTTCCTTAAAGGGAGTCTCCCACAGTTCCACCTACTGTGGGGGGAAGGGG | |
| AAGGGGAAGGGCGGAGCTCCCTTGCTGTTCTTCAACCACCAGCCAGTACTCCCGTGCAGGCTCAAGGG | |
| CAGCCTGTGCTCTCCACACAGCCAAGACCTGCTTGCTTGTTACTCAGTTTTTCTTACACAGGCCGTTA | |
| GCTGATTAATTGGGTTTTTATTTTATGTGTATGGATGTGTTGCCTGTGTGCATGAATGTATACATGTG | |
| TGCTGGTGCCCGAAAAGGCCAGAAGAGGGTGTCAGATTCCTCTGGAAATGGAGTTACAGGTGTCATGT | |
| GGGTGCTAGAGTTGAACCCGAGTCTTAACCACCGAGGCATAGCCACTGATTCAGCCAGACTGACCAGC | |
| CTGCAAACCCCAGGGATCCTCTGTATCGGCCTTGCCCCACACCTGCTAGGATTACAGGTGGTGGTGGG | |
| CTTGGCTTTGTGGTTGCTGGGGAACTGAACTTAAGACCTCAGTATGTGCACCAAACCCTTCTACTGAC | |
| TTAGCAACATTCCCGTGGAAGTCCTGAAAATGAAGGACGGGGGGAAGGATACTGACTCTGCTGTAAGA | |
| AAATTCTTACATTTATGTTATTGTGTGGATGTGTGTGCACTCAAGCACAAATGAGAGCTAGAGAGGGC | |
| CTGCAGAAGTCACTTCTCTCCTCCCACCAAGCGGATCCCAGGGACTGAGCCCAGGGGTCAGGCTTGGT | |
| GGTGAGCGCCTTCGCCCACTGAAGCACCTCACCAGCCTGAAAATAAAGCTCTCATGGCACCCAGCCAC | |
| GTCTGCTTTTTCTCATCCCGTCGCTGGCTCATTTCCCCCGATAGCAGCTGGTAGAGTCATTATAGCAA | |
| GACCGTGCACCCTGTAAGGCCTGAAACACATACTGACCAGCCCTCCACAGGTCCCAGCTGACTCCTGC | |
| TGGGACCACTGAGTTATAAATCAGAGCGTCATCTACCGGCTGCAGAGGCGACAGCTTTTTGGTGTCAC | |
| CAACAGCAAACACTGTGCTGTATTCCTGTGCACTCACCACCTGTGAGAAAATGCACCAGGGCAGGAGC | |
| TCAGGCCTGCAGCTTCAGCCAGGTTAAGGCCGGCCTGAGCTCTCCTTCCAGGCACAGCCTTGCACTAC | |
| TCTAGCTGGCATCTGTAACTACCGCAGTCCACTGTGCCCATCTCTGCATGCTACAGCCCTCACTGTCC | |
| TTCCTGGATGTCAGTTTCCATGGGAGACAGCTTCGCCTTTCTCCAAAGCACATCCTAAGTCTCCTTCC | |
| TACCCTGTCCCCCCAAGGGGGGCTCCTCCTCCACGGACACTGTGCTTTCAGTCTTTGCCAGGGGTCTG | |
| ACCTAGGCCTGGGCCGCACCAACACTGCTAGGACCTGGCAGCACCCACTCTTCCTCCTTCAGGAACCA | |
| CGTCCTGACTTTCCTCGCCCACAGGGCTTCAGTGTGACACTTGTCACAAGGTGACAAGTCCTCCTGCA | |
| CACTGGTGGGCTCTGGGACTGACATGAGATCATGTGCCAGTGTCACACAGAGAGCTGTGGCTCAGCCA | |
| CTGAGGGGGGTAGGGCTACTGTACCCCACCTAGGCATTAGCCCTGCTAAGCACCACAGGGAGAGACCA | |
| GACCCCACCAGGAGGCCAAAGCGGCTGGTGGCATTAGGGATCCACTGCCAAAGACTGGAACTCTAGGG | |
| CTTGGACAGACAGATGGCTCAGTTGGTAAAGTGTTCACCACACAAGCTGCAAACCTGAGTTCAACCCC | |
| CAGCACCCATGTAAAATGCCAGGCATGGTGGAGCATGTGTAATCCCAGTACTGGGGAGGTAGAGACTG | |
| GAGGACAACCGGGGTTCACTGGCCAGCCAAACTAGCCCAATCGTGGAAGCTACTGAGACACCCTGACT | |
| CAAAAATCAAGGTAGACGGCTCCTGACGAACATTCGATTGACCTCTGGTCTCCAAACACACCTGTGCA | |
| CGCACACATGCACACACAAACATATGAAGGACTGAGATTCTGCATACGTTAAACAGCAACTCTCTCCT | |
| CCCCCTTTTTATTGCATTCATTTACTGGTGTGTGTGGCTGAGAACAATCTATGGGGTCAGTTCTCTGG | |
| TCCACCAAGTGGGTCCAAGGAATCAGACTCAGTTGTAGGCTTGGCAGCAAGCACCTTGACCCACTGAG | |
| CCATCCAGCCAACTCTCTTTTTTGTTTGTTTAGTTTGTTGGGTCAGAATCTCTCTGTGTAGTCGTGGC | |
| TGTCCTGGAAGCCACTCTGTAGACCAGGCTGGCCTCAAACTCAGAGATTCACCTGTCTCTGCCTCCAG | |
| TGCTGGGAATAAGGCATGTGATAGGGTTAAACCCACCACACCCAGCTCTACTCCCAGGGTTGTTAGCG | |
| CCTGGAAACCACATTGGATGCTCTGTGGCTATGACTTTGGGAGCGCTATTATTTTCATTACGCTTGGT | |
| GTCTCACACAAATGGAAACCTCGCACTTGTCTTTGAGTGGCTGACTTGCTCCATGTAGGTCCTTAAGT | |
| CTCATCCAAATCTGGCAGGCGGTGGTGGCGCACGCCTTTAGTCCCAGTACTCAGGAGGCAGAGGCAGG | |
| CGGAGTTCGAAGCCAGCCTGGTCTACAGAGACAGTTCCAGGACAGGCTCCAAAGCAATACAGAGAAAC | |
| CCTGCCTCAAAAAACCAAAAAAAAAAAAAAAAAAAAAAAAAAAAAAAAAAAAAAAAAGAAAGAAAAAG | |
| TTTCATCCACATCGTTAGACGTGTTGGTTTCCTTCCCTGTTAAGGATGAGCAGCATTCTTTTATCTGT | |
| ATAGACCACATTTTGCTTATCCAGCCAGTGATGGACTTCCTACCCTCCCTGGTCCTGAGGCTGGAATT | |
| CTCATACCAGACCAAGTGCCACAGGCTGGAGTACTAAGCAACACCCAAGTCCTAGGCTACTGTAAATA | |
| CGCCTGCAGGTGGCTTTCTCTTTGCCCAACACCACAAAACATAAAGAAGGGAGCCAGACATAGAGGCA | |
| ACTACTACAATCCAGACCCTCAAGAGGCTGAAGCAGAAGGATCCAAAACCGTACTACAGTCAGTTTCG | |
| CCGCAGCCGAGGCCAACTAACTAATTAATTAACAAAATAATACGTATTGTGGGTGTGCATCTTGCGAT | |
| GCTTGTGTTGTGGTGTGGGGATTGAACCTGCCTACGCTCAGCAAACGCTCTGCCAGTATGAAGTGTCC | |
| AGTCCTCTCCCACACACACGCGAACACACGCGAACACACGGGAACACACGCACACACACACACACACA | |
| CACACACACACACACACACACACACGCACACGTATATTTAAGATCTTTCCTCTCTCTCTCTCTCTCTC | |
| TCTCACACACACACACACACACAGGGTTAGTTAAGACCTTATTTGTATTACTTTTAATTGTGTGTGCG | |
| TGTGTCTGTGCAGGGCACGCACACACGTGTTTCAGTACCGGAAGAGGTGTGGGATCCCCCAGGTGCTG | |
| GAGCTACAGTGAGCCAGACACTGGTGCTCTGAACTGAACTGAATCCTCTGCCAAAGCAGAAAGCACTC | |
| TCTGGACTCCTGCTTTTGTTGGTTTTGTTTTGTTTTGTTTTGTTTTGTTTTGTTTTCTTTGTACTTTT | |
| CAACAGATTCTCACTAAACTGTCCAAGTTGGCTTGAACTCCCTCTGTAGTTCAGGCAGGCCTTGAACT | |
| CACAATTCCTCAGGCCTGGGAGAGCGGAATCTTTTCGTCAAGAAAACACCCCATTTGAAAACTGAGGA | |
| ATGCTGTATCAGCACAGGAAGGGGGAAGCTCAGGCCTTGTGGCTTAGAGAAGCGGCCTTGTGCCATGG | |
| GGTTAAGAGCCCCAGGCTGCCCCATCTCGTTGGCTGAGGGAGGCGCTTCCCGTTATCTGAGCAGAGCT | |
| TCCAGCATCAGCAACATAGTCTCCAAGTGGCTGAGGAATGGAAGGAGGATGGACTGGAAAGGAGAAAA | |
| CAGGAAGGGTACTGCCGCCTGTGTGTGGGGAAAGGGGCAGAGCTGGACAAAACAGTAAAGGCGTCTAT | |
| TTAAAGTGTGCGATTCCATCTGCACGAAATGTCCGCAACAGACAGATCCCTAATGAGAGAAACCTTAG | |
| AGGCTTGCCCAGGGATGGGACAGGGGACTAGACTTTTGAGGGTGACTTTAAAATGCTCTGAAATCAAT | |
| GTGGCATCGACTCCGACAACTCTGTGGACCACAGCACAACCAGGAAGTGCACTTTGGATGGGCAAACT | |
| TCTGGGCATATTAATTACAGCCCCAAAGGCTGCTTTGTTATAAAAAGCACTGGTGGGCTGGCCGGCTA | |
| GCTCAGCAGGTCAGGCGCTTGCCACCAACAACCTGAGTCCCAGACCAAGGCCACATGGTGGAAGGAGA | |
| GGCCTGCCAGAAATTGCCCTCTGACCCCCACAGTGCCGAGCACTCACACTCATGATAAACTAAATAAA | |
| TCTAAAAAACAAAACAAGACTTCAAAAGCAGCAGATGGAGCGCTGACACAGACACTCGGGAGGCTTGG | |
| GCAGGACGGCTCCAAATTCAAGGCCAGCCTGATCTACGCAGTGAGTACCAAGCCAGCCAGGACTTCGT | |
| AGCAAGACCCTGTCTCAAAGACATAAACAGGGCTAAAGGGCTGCCTCAGTGGTTAACAGCGCTGGATG | |
| CTCTTCCTGAAGACCCAGGTTCAATTCCCAGCACCCCGGGCAGGCAGCTCACAACCATCTGTAACTAC | |
| ACTCCCAGGGATCCAGTGCCATCTTCTAGCCTCCGCAGACACCAGGCACACATGGAACAAAATACCGG | |
| GACATAAAGAACACACTGTGTGGTGAAGCCCAGGGAAGGATCTGTGGTGGCTGTCACAGTACCGAGGC | |
| GACTCTTCTGAGTTTGAATCAGGGGACGGGAAGGAGAGCTCAGCTCAACCGCTGCTACCTGTGGCTCC | |
| TGACCACTGCCCTTCAGCTCTTGGTGCCCACTGGCTACCAAGCATTCCCAAGTGACTCGCAGTCACCT | |
| GAAATTCAATATGCCAACATGGTGAACCCACTGTCTCTCCATCCTGCGTAGCAACACGCAAGGACGGG | |
| GAGCCAAGACTATGCCTCCCATGAACTATCTGTCCTCTGTCCCCGCTTATCTCCTAACTGGACAGTCC | |
| CCAGTCTGGAACTGGTGCCTTATGTTCCTGGAGAGCCTGCAAAGCTGCCTGTTTGCTGATCCCTTTCC | |
| TTCCAGACCCTGCACTACAGAGCTGAGAGCCACCCAGCTATAACCCAGTGTTTCGTTTGTAGCTGACA | |
| GGGACTCACAGAGCCCAGGCTGCTCACAAACTTACTATGTGGGGAAGCCTGACCTAAACTCCTGATCT | |
| TCCTGCCCTGCCTCCCAAGGCTGGCTGGGATTACAGGCCTGTGCCGGGACACCTGGCCGGGACACTAG | |
| CTTGTCAGGCAGGCAGAGAGGGCTCTCAAGCCCTGTTAAGAACTTGCTATTGGGAACACACACGCCCC | |
| ACCCAGGAAAATGAATAGGACCCAACATGGAGTTTCAAGGGGCATGATGGGAGCTCAGGAGAGAATCC | |
| TCTGCATGCTCCAGTGCCTCCTAACACGAGCTGGGTCTAGCCATCTTGCTGCTTACTCCTCGACAGGC | |
| CCTTGCTGACAGCACCTCCCTCCTTCAGTTCCTCAGACACTCACAGCAGTTGGGGCTCTTACTCTGTG | |
| TCTGGCAGTGTCTCACTAGACCCTTGGCAACCCACCCTGGGGACACGTACCACCCCCACTTCACAGGG | |
| AAGGAAACTGAGGCACAAAGAGCAAGAGTACAAGGAAATGGGCTGGGCCTTTGAGCCCAGACTCCCAG | |
| ACGCCAAAGCTCTCGATCCCACAGGCCCACCTCGGCGGGCGATCTCCGCCTTCAGCAGCCCCTCCATG | |
| GCATCCGACTCAGCCAGGTCCAAAGACAGGTCTCCATCACTGTTGACGGCGGCGATGTTGGCCCCATG | |
| GCTCAGGAGGTACCTAGGGGCAGGGGAAGGTCAGAGCCACCAGGCCTGGACCTAACGCCTAACCCAAG | |
| CCCTGCCCTTCAACCCCAGCCTCACCTGGCAATGTCCAAGTACCCACAGGAGGCTGCCACATGCAGCG | |
| GCGTCCAGCCCTCGTTGTCCGCCTGGTTCACAGTAGCACCCTGCTCCACCAGGAAGCGCACCACCTCC | |
| AGGTTCTCGTCTATGCAGGCCTGGGGACGGGGACAGGCCCATCAGCTCCCGGCCGGGCCAATGAGAGG | |
| TGTGGAAAGCAACGCCGATGGGCTGCAGCACAGATTCCAGGGGCCCTCTGGTCAGTGGCCGCCTAAAA | |
| TATGCCTCGTTACCCATGCTTGGGTAATCTATGCATGCAGAGCTCATGGAGACTAGAGCAGGCTCCAA | |
| AAGGCAGATTGAAAAGGCGACCAGGGAAGAGGCGGAGCTGCCATCCCTGCATGTGACTGCTGAACATA | |
| CCCTATGAGGCAGAGGAACCCCAGAGCCCAGCCATGTTCTTCCAAGGGGCAGGGCAAGGCTAGGTTGA | |
| GGCAAAACGCTCACCTAGCCCTGGGTTCCATCCACAACACAGGAAAAAGAGAGATCACCACAAAGAGA | |
| CACACGCACATCCCAGAGTTGAGGGCTTGGGGCACAGTTCCCAAAAGGGATGAGTAGGCTATGTTCCC | |
| GGTGTCCAGGGATCCAAGCAGACCAAGCTCTGGGTCACTGAGGGCCTACCGTGCACAGGTCTCCTCAG | |
| AACTTCTTTTCTAAACACCCCACACCACACTAATCCCCCACCTCCTCACCCTTCGAACCAACAGCTCA | |
| GGAGAGGAAGGCCTCACCCTAGCCAGCACAGCACCCAGGGCCACAAGAGAGCTGGTCTCAATCCCTGA | |
| TGACACTGCTTGGACACTGGAACCACTGAGTCTAGGGAGGGGGTTAGGTCCGGACTCCTGGGTCCTCA | |
| TGAAAATTAACCCCCTTCTACAAGCGCGACCATCTGGAGAAAGAGGGAAGGAAGCTACGAGGGCCAAG | |
| TGCATGAAGTCATGGAAATTTAGGCTGGGGGGGGGGGCACGTGCCCTGGGAACGGGATGAACTCTGGG | |
| CTTCACTCTGGGCTCAGTTTATTTCCACCCTGTTGTCATGGTGATGGGAGGGGGGGCAAGGAGGCAGA | |
| TGGGCCTTTCCCTTTCAAGGACCTGGCCGGGTACGGGCATCCATGTGAAAGATGCCTGAGGCTGGGCA | |
| CTGGGGACCCAAGAATCCTCCTCCCTCAGATGTAGAACTCTAGCCAATCCTCTTTCCTTAGACCCAGG | |
| GATCCAGACTTGGCCCTCCTCCCTCAGGCCCAGGTGCTAGGGCTCCCCATCTCTCCCCTGCTCAAACC | |
| TAGGACTCTTAACTCCCAGCCCTACCTACTCCAGACCCAACTCATAGCCATTATTGGACAAGGCAATT | |
| ATTGGACAAGGGAAAGAGGAAGGAATGTCCCTGCCTTGCTAAGGCAGAGGCTGGGGCTTAGGAAATGT | |
| CATTGCAGGAGGCTGATGCCCCAAGGAGGGTCTAGAACCGGAAACACTAAAAAGTCTGAGGTGTAGAA | |
| ATCACCACAGACTGGGTGGCTCAATGCCCCTGCTTTCCTGGGACTGAAACTAGTTTCAGGAGTTTTCA | |
| CTGCTGAAGCCAGGGCAGTGGTACTAGGAGGTGATGCTACGTACGCACCACTCCAAACCCCAGCCCCC | |
| TCTGCGTTCTGGCCCTGAAAGCCAAATGATCTCACTGAATCTGATCTCCAGTCTCCCAAGCCTCCTGC | |
| AAAGGCCTGAAGAGTCAGGTCACCAAGGTGTCTGCATGGCGGGAGGAGAGTCCCACCTGGAAGGCTGA | |
| CACGTCAGGCCTGAGGTCACAGGTTCCTGTCAGAGAGGATGCTCTAGGGACCTCCAGCAGATGCAGAG | |
| GAAGGGGATGCAGTTGGGAGGGAACTCTTGGGAGGGCCAGGGACTTTGGTGATCATGTGAGCAGCCTG | |
| AGCTGATCTCCTGGACTGGTCAAAGACGCTGACACCCTGAGTGTGGCCTCGGGAAAACAGGACCCTGC | |
| TATATATAGAGGACGATGTCCCACACAGCTCACGCCGGCCCCATAAAGGAAGTTTTCCACAGGACGCC | |
| TCTCACCATAGAGTCCCTCTGGGGACAGGGGTGACCACTGGTCCCATTCTACAGGTAAAAAAACTAAG | |
| GCGCACCGAGAAAAGACACTCAAGATACAAGACACAAGAAGCAGACTGACACAAAAAGTCAGCACGGA | |
| CTTTTTTTGTTTGGTCAAGATTTTGCAACTGGGTCTCATGTGGGCTATCAAGATGGCCTCAAAGTCAC | |
| TGTGTAGATGAAGGTGACACTGAATTCCAACCCTCCTGCCTCTACTTTCCAAGCACCTGATTTCTGTG | |
| GTATTGGGGTTGGAACCTGAGGCTTCCTGCACTCTAGGCAAGCACTCTGTCTAAAAGACAGCCCAGCC | |
| CAGGACAGACGGATTCTGTTTTTCCTCTGCCTGGATGAGTGAAACACTGAACCTTTATTCCCCACCTC | |
| CACGAGCATCCTAGCAAGAGGACGACAACCCAGGAGATGGAAGTTGCCATGAAAGACTGAAAGTGAAC | |
| CAACACTGTGGCCAGGAGGAAGAAGAACGGGGATGGGGGCTCTGCTGTGACTAATCTTGTCCCTGACA | |
| ATGCCAGCTTTTGGATGACGGGGAGATAAAAGCATCCCGAATCCAGAAGGATCCCGGCAATACAAGAT | |
| GGTCCTCACTCTCGGGGCACAGATCACTGGAAAAAGATAATCACAGTGTCTGAGTCGCCCAGGGTCCT | |
| GGTGGGGTAGGTTCTAGAAGGTGACAGGGTGGAAATCTAAGAGACAGGGCATAGTTTTTAAAGCAGGA | |
| TGCTGCCCAAATATAGTCCATGGGGTGGTAGGTGGAGTGGGCATGCCTGTAATCCCACAAATGAGGGG | |
| GTCAGAGACACAGGACCTGTATTTGAGGTTGGCCTGGGCTACACAGGGGAAAAAACAAAAAACAACAA | |
| CAAAAACAAAACCAGAGGAGAGAGAAATAGGGCTTGGAAGACCGAATGCTTAGAAGTTTCCAGAAAGG | |
| CAAATCAATGTGGACACAGAGAGAGAAAGACAGAGAGAGAGAGAGAGAGAGAGAGAGAGAGAGAGAGA | |
| GAGAGAGAGAGAGAGAGTTGAGGAGAAACCTATGGGGGGTGGGGGTGGGTGGCAAGATCACCAAAAGG | |
| GGATGAGCCGAGAATTGAATTAATAGGACATGGGGAGGGGAGGAAGGTTATGGGATGGGGCCCAACAG | |
| ACGGTGGAGCGCCTCTTCTCCAGGGGAACAAAGGGGTACACTGCCTTGGAGGGGCAAAGGACCCTCCT | |
| GAGGCCACAGCGGACAGCACGGGTCACAGGAAGTGGGGTAGGGAACAAGGTGGACCCCCCAAAAGAAG | |
| TGACACTGAAGGGCCTGGGCCTGGCTAGCCTCAGAGGAGGGAGTGGGGGATTGGGGGGGGGGGCGTCA | |
| AGTCAGAGCTGGGCCCTGGAAGCCTGCGGCACAGCCAGGGCAGCCACCAGCCTGGAAAGGCACGGGGT | |
| GTAAGCCATCCGTGTGCGGAAGACGCCGCCGGGGAGAGCGGTGACAGCGCGGATGACAGGGGCGAGGC | |
| GGCCCCTGCAGGGCAGGAGGCGGGGAGGGAGGAGGGGTGGCTCGGGGGGCCCCGGGGAGGGAGGATGC | |
| TCGGGGGCCGCTGACCTGGTGCAGGGCGCTGATGCCGTCGGCGTTGGTGGAGTCGAGCACCGCTCGGG | |
| CAGGCGGCGGGACGCCGGCGTCCGATTCCCCGCTGGCGCCGGGATCGGGGCCCCCGGGGTCCCCGGGA | |
| TCCCCGGGGTCGGCTGCGCGAAGCATCAGGCGAGCCTCATCCAGATCGCCGCCCGCACAGGCCGCCAG | |
| GAACTCGGCGGCGCGCTCGAAGCGCACGGTGCGAGCACGACGCTCCCCGGGGCCCGGCTCAGCCCCCG | |
| CCCGCGCCCCCCACCGCCGCAGCTGCTCCTGCCGCCGCTCGCGGGCTGCCGCCGCCGCCGAAGACGAC | |
| GACGACGACGACGACGACGACGACGCCGCCCCGGGGCCGTCCTCGCCCGACATCGCGCCCCACACCGG | |
| GCCGCTCGCCCGCTCACCCACCGAGCGAGCGAGCGAGCGAGCTGAGCGAGCGCCCGCCCGAAGGCCGG | |
| CCGGCGACGAACAGCCGCCACCCGCCCGCTCGCTCGCTCGCCCGCCCGCCCGCCAGCCCCGGGGGCCG | |
| CCGGGAACCGCCGCCGCCGCCGCCGCCGCCGCCACAAGCACCGCCCCGAGGCTCAGGCTGGGCCCCAC | |
| CCCTCCCCCCACGGACGGGCGTTGACGTCACGACGCTGCCCCACAGCCCTCTGGGAAATGGAGTCCTC | |
| CGTTGAGAAGCCCGCAGGGTTTTTTCAGCAGACTCGCTAACTGCTGAGGGAACGGTCGGGGTGGCACG | |
| GAAGCCGCCAGCAGGCGCGCCTACAGCCCCCAGCACCTGAGAGGCAAACTGCTCTCTCGAGTTCGAGG | |
| TCAGCCTGGGCTACAGAGGCAGTGCCAGGGTAGCACCAATTGCCTAAAGCAGGACACGCCCCCCCCCG | |
| GGAATGCTGGAAATCTGAGTTTAGAGGCGGGACGGGATGCCCGGGGGGATGCTGGGAGATGTAGTTTT | |
| TTTGGTAAAGCGGCGCAAAGGATGGCGCGTGGGAAATGATGGCGTGTAGCGGAACCCGAGAGACGCAG | |
| AAATAAGACTCGCGTACTTTCAGTTGTGTTTTTGCTGTGAGATGGGTTTGCCCTCGAGCTCGCTGTGT | |
| GACTGCGATTGTCTGTTTTAAACTCCCGACCTTCCTGCCTCCGTCTCCTAATTGCTGGGGTTGCAGAC | |
| GTTTGTTTGGGTTTTGTTGGTTTGGTTTGGGTTGGGTTTTTTCTTGGGGGCGGGGGTATTTTGTTGTT | |
| TTGTTTTTGTTTTTGTTTTGAGACAGCGTCTCACTATGTAGCCCTGCCTGGCCTGAAACTCGCTACGT | |
| AGACCAGGCTGGCCTCGAACTCATAGAGACTCCTCCCCCCACACTTCTGCCTGGTATGAAGGGGGCGC | |
| CACCAGGTCCCGCTTGTTTTGGTTTTGGAATCTGCCCCTCCCTCCCTCCCCATCAACACCCGATGAAG | |
| GACAAGGATTTGTGAATGAATGAATGAATGCATGAGTGCATGAATGAATGGGCTCCCCAAGACGTCGG | |
| GGAGACCAGGGGCCCACGGGAAACTGAGTCCTGAAACCAGATTAAACACCAATCGCCGCCAAACTCCT | |
| CTGGGTAACTAAGGTTCCCGTGCAAAATCCAAGGGTATCGGGTAGCATGGGGCAAGCTGGGAAATGTA | |
| GTCCCAGGGCCACGCCTCCTAAAGAGTTCAGCCCCCAGACTTCCAAAACTGCCTGAGATGCCAAGGTA | |
| CCCCGGAAAGTCAGTTTCCAGATGAAGACAAGCCTCCGGTCTCCAGCGGTAATCCCTTGAGCACCCGG | |
| GAAGAAGGGTCCCCAAAGAACCACACATTTCTCCTTAGCCCACTCGGGGCTGCGGGGGACGCTAGGAG | |
| ATGCTCTCCCGGCTGCATCAATGCTCTCCTGGAATTCTGGGATCGGTAGCACAAAATGTGATGCTCCG | |
| ATAGGTTTGGAAGTTTTGTTAGTAGACCCAACAGATAAAAGAACACCTTGATCTTTCAAGAATCTTCC | |
| CCCCACCCCCACCCCCACCCCCACCTCCACCCCAAAAATTGCAATTTGAGAAGGACAGAAACACTTTT | |
| GAGACAGGAACACAGACTCACACACACACACACAAAAAAGTAGAACAGAAAGCTGTCAAGTTTATAGA | |
| GAGAAAACACGTCTTCCTAAGGGTCGTTAGGGCAGCCCCGTTCACACTGTGACCCTTGGATTTGTGAA | |
| TGAGAGATAAATTACAGACCCTGGCAGAGTCTAGGGAATAACGACCATAAATCCAAAAGGATAACCCT | |
| GTGGTTTTTAAGATGTGAGATCACACACACACACACACACACACACACACACACACACACACACACAC | |
| CATTCTTCCCCAAGGCAAGAAATCAGATATTTCAACCCCTGGGGTCCAGAAGGAAGGAGGTCGCTGAC | |
| TCCAAAAACTGTCTTCTGATTTCCACCATGGATTTCCACACACACACACCCTATCAACACACACACTA | |
| AATAGACGTTTATAAAATGATCCACAAAATAAGGCTACACCAACACACAGAGGTAAGACTGTTGTTAG | |
| ACAGTTTTGGTCTGGTTGGGTTTTTTTTTTTTTTTTTTTTTTTTTTGAGTAGCCTTCTCCTGTCCCAT | |
| TTCTCATGCCTCTACACACACCTGGCCTCTGGGTGTGTTATTTTAAAACATCCTTAGAAGAATTAATG | |
| ACCTTGTACAACCAGTTTAAATGCAAGAGGCAATTAATTTTGTTTTGTTTTGTTTTTCGAGACAGGGT | |
| TTCTCTGTGTAGCTTTGGAGCCTGTCCTGGCACTCGTTCTGTAGACCAGGCTGACCTCGAACTCACAG | |
| AGATCCCCCTGCCTCTGCCTCCCGAGTGCTGGGATTAAAGGCGAGCCCGGCAGCACTGGAGATTTAAC | |
| TCAAGGTCTCCTGAGTGCTCGACAAGCTACTCCCAGCCATGAACTTGATATCTCTTTAATGGCAGCTG | |
| ATGTCTCTCCCGGGCAACATGGAGCTGTCCAGCCAAGCCGCACAGCCAGCCACGCATAATGACAACAC | |
| GGAAGAACTCAAGCGGATGTCTGGAGGGCCTTTATTTTGAGTTACAGATGGGGGACACACTCCAGAGG | |
| CTCCCAGGCTCCATGCAGTGGGGCGTGTCCTGGCAGTCTCACTTCCAGCGGCCTCCAACTCGACCCTT | |
| CCCAGCCCCCTTTCGGCTGTGGGAGAAGAAGGTGGAGTCAGGAAGAAGCCCGGAGCCTCCGAGATAAG | |
| CTTAACACAGTCCCTTTAAAATTAAGGAAGTCCACCAAATACCCACCCCCACCCAGAGGGAAGAGAGA | |
| GCAGAGGTCAGCAGAGCTGTTTTTTTTTGTTTGTTTTTTGGGTTTTTTTTTTGCAGTAGTGAGCATAA | |
| AGTCAAGGCCTCACACGTGCTAAGTATGTTCTGTACACTGAGCCACGCCCCTTGCCTCTCACTGGCGA | |
| TTCTAAGCAAGGGCTCTACCACTGAGCCACATCCCCAGCCCCTCACTGGGGGATTCCAGGCAGGGGCT | |
| CTACCACTGAGCCACGCCCCCAGCCCCTCCTCACTGGGGGGATTCTAGGTAGGGGCTCCACCACTGAG | |
| CCACACCCCCAGCCCCTCCTCACTGGGGGGATTCTAGGCAGGGGCTCCACCACTGAGCCACGCCCCCA | |
| GCCCCTCCTCACTGGGGGGACTCTAGGCAGGGGCTCTACCACTGAGCCACGCCCCCAGCCCCTCCTCA | |
| CTGGGGGGACTCTAGGCAGGGGCTCTACCACTGAGCCACGCCCCCAGCCCCTCACTGGGGGATTCTAG | |
| GCAGGGGCTCCACCACTGAGCCACACCCAGCCCCTCACTGGGGGATTCTAGGCAGGGGCTATACCACT | |
| GAGCCACACCCCCAGCCCCTCACTGGGGGATTCTAGGCAGGGGCTCCACCACTGAGCCACGCCCCCAG | |
| CCCCTCACTGGGGGATTCTAGGCAGGGGCTCTACCACTGAGCCACACCCCCAGCCCCTCACTGGGGGA | |
| TTCTAGGCAGGGGCTCCACCACTGAGCCACGCCCCCATTAAGGGCATCTCTTTCAGATAATTCCCAGT | |
| AGGGGGTTGGTGGCCATGTTGGAGTTGACTTTCTTGGGTTAGTTCGGAGAACACATGCAAATTTATGA | |
| GTAAGGGGCCTGAGGGAGAAGGAAGGGTGAGCTGGAGTTGGTGACTTGCATGCAACAATGTTGAGTGA | |
| GGCTGGAACAGTACAGAAAATGCTAGAAAAAGGCAGAGACTGAGCAGTGAGCGGCCTGGATACGGTGG | |
| AGCACATCGGTAATCTCTGCACTCTGAAGGGGATGAGGCAGGAGGATCACCGAGAGTTTGAGGACAAC | |
| CTGGGCTATATAGCAAGAGCCTGACTCAAATGAAAACAACAACAACAGCAAAAAAGTCGGGTATGATG | |
| GCTCTGTAATCCCTGAACTTGGGAAGCAGAGGCAGGAAAGTGTCAGGAGTTCAAGGACACCCTCAACT | |
| ACAAATGGAGTTCAAGGTCATTCACGCTTACAGGAGACCTTGTCTTAAAGCAAGAAATAGAAGGAAAA | |
| GGGGCAGGAAGTGGACAGACAGATGGAGAAGGGGGGAGGGGGGAAAGAAAGGAAGAAAGAGAGAGAGA | |
| GAGAGAGAGAGAGAGAGAGAGAGAGAGAGAGAAAGAAAGGCAGACAGAGGGGGGCACTGAGATGGCTC | |
| AGCAGGTAAAGGAGCTTGCAGCCAAGCCTAGGCCCTGAGTTTCAACTCTGGGACCCACATGATAGAAG | |
| GAGAAAACCGACTTGTTCGAGTCATCCTTCGGCCACATCTGCACCATAACAGCACACACACACACACA | |
| CACACACACACACACACACACACGCACGCACGCACGCACGCACGCACGCGCGCGCACACACACACACA | |
| CACTATGCGGTGTGATATGATACAAAAAAAAGTGTAAAAGAAAATGTACTCAGAAAGAAAGGGTTGGA | |
| GGGAGGCAGAGAGGCGGGGAGATTGAGACCAAAGAGTTGATAAAGAGAAGCAAGAGATTGGAGTGCAG | |
| GCCAATAAACACAGCACTCAGCAGGCTGAAGCCAGGGGACCAGGAGGAGTTCAAGGTCAGCCTCAGCT | |
| ACCTAGTGAGACTGGGCTGCATGAAACCTTGCCTTAAAAATAAATAGACAGAGCCGGGCAGTGATGCG | |
| CACGCCTTTAATCCCAGCACTTGGGAGGCAGAGGCAGGCGGATCTCTGTGAGTTCGAGACCAGCCTGG | |
| TCTACAGAGCTAGTTCCAGGACAGCCTCCAAAGCCACAGAGAAACCCTGTCTCGAAAAAACCAAAAAA | |
| TAAATGAATAAATAAATAAATAAATAAATAGACATACCAAAAAAAAAAAAAAAAACAGGAACAGTGAG | |
| TCATGCCAATCATCCCCACATACATGGGATTAAAGCAAGAGGATCTCCTACAGGTTCAAAGAAAGCCT | |
| GGTCTACATAGTGAGTACCAGGCCAGCCTGGGCTACAAAGTAAGACTTCCTCGAAATAATAAACAAAC | |
| TAAACAAACAAACAAACAAATAAATAAACAAACCCGAGAGAACAGATACAGAAAGGATGTCTCAGGGA | |
| GCAAGGAACAAAGACATATAAGATGCCAAAAGGAGGGCTGGAGAGATGGCTCAGCAGTTAAGAGCACT | |
| GGCTGCTGTTCTGGAGGTCCTGAGTTCAATTCCCAGCAACCATATGGTGGCTCACAGCCATCTATAAT | |
| GAGATCTGGTGCCCTCTTCTGGCCTGCAGGCAGGCATACAAGCTGGCAGAACACTGCATACATAAATA | |
| AATAAATCAAAAAAAGATAACACTTTAAAGAAAATGATACTTTGAGAATTCTATGTATAGAGCCAGGC | |
| GGTGGTGGCGCACGCCTTTAATCTAGGTCCTCAGGAGGCAGAGGCAGGAGGATCTCTGTGAGTTCGAG | |
| GCCAGCCTGGTCTACTGAGCAAGTTCCAGGACAGGCTCCAAAGCTACAGAGAAACCCTGTCTCAGAAG | |
| AAGAAAAAATAAAAGATCCCAAAGGGCAGTGGTATGCAGAAGACAGGGAGGAAGGGAGGGAGGGAGGG | |
| ACAGAGGGAGGGACAGAGGGAGGACAGCAGGCCTTTTGTGGAAGCAGCACTTACAATTTCTGGGCATG | |
| GCTGATTCGGTTGTACAGCACATTGATCTGTAGAACGAGAAGCCAGGCTAGGTGCAGATGTCCAACCA | |
| AAGCCCTGCCCTGCCTATCACCCCTGTCACCCCAGCCTGGACCCCAACAGAGGCAGGTCCCACCTCGT | |
| ATTTCTGTTGCTTCAGTTTCTCCATCAGATCAAATTTCTCTGACTCGAGCTGGTGGATCCATTCCGAC | |
| AGCTCCTGGGCCTTCTCCCTGGCAGAATGAGAATAACTGGGATGCAGCGAGACTATGTTCTGGGCCCA | |
| GAAAGGTTGAGACACCTACCCCAAGCCTCAAGGCAAGTCTCTCTGAGCTTGATACAGTTGGTATGCTA | |
| AGCCACTATGAGCCTGATACAGTTGGTATGTTGTGGAATGTTACTTTAACTATGTAAAGATGCCTTAC | |
| ATTTGTTTACTTTGTGGAATGTTACTTTAACTATGTAAAGATGCGTTACATTTGTTTACTTTGTGGAA | |
| TGTTACTTTAACTATGTAAAGATGAGTTACATTTGTTTACGCTGTGGAATGTTACTATAACTATGTAA | |
| AGATGCGTTACATTTGTTTATATTGTGGAATGTTACTTTAACTATGTAAAGATGCGTTACATTTGTTT | |
| ACGTTGTGGAATGTTACTATAACTATGTAAAGATGCGTTACATTTGTTTAGGTTGTGGAATGTTACTT | |
| TAACTATGTAAAGATGCGTTACATTTGTTTACTTTGTGGAATGTTACTCTAACTATGGGAAGGTGTGT | |
| TGCATTTGTTTCTGCTGCATTTGTTGAGTTGGATAAAGGTGTGTTGCTGTTTCACCTTGCCTGCCTAA | |
| GGCACCTGATTGGTCTAATAAAAAGCCGAACAGCCAATAGCTAGGCAGGAGAGGGATAAGCGGGGCTG | |
| GCAGGCAGAGAGAATAAGTAGGAGGAGGAATCTAGGATGCAGGGAGGGAGACCAAGGGAGAAAGAGAG | |
| GGAGATGCCTGGAGCCAACCAGACATGGAGTAGTCAAAATGCAGATGAAGAGAAACAGGTTAATTTAA | |
| GTTATAAGAGCTGGTAGGACAAGCATAAGCTAAGGCCAAGCTTTCATAACTAAATTATCTCTCCACGT | |
| CTTGATTTGCGAACCGGTTGGTGGCCCGAAAGGAAGGCAGCTACACTGGTAAATTCTTGTTAAGATTG | |
| GAGGCTGAAGTTTTAAAATATGGCCACTTTCTGAAACGGAGGTCTCCCAAAGGAGAGAAGGAAGTGAT | |
| GACTGGGAGCCCCAGAATCGGAAAGATGTTTGGTTTTTATTTATTGCTTATGTAGTGTGTGTGTGTGT | |
| GTGCGTGTGTGTGTGTTTAATTTTGATTTCTGAGACAAGGTCTCGTGTAACCCAAGCTTCAATATATA | |
| GGAGAGGATGACCCAGAACTTCTGATCCTCCTGCCTCCCCCTCCTGAGTGCTAAGATTCCACCTAAAT | |
| GAGGGAGAGAGCACTGTCAATGCCAGACTCCACAGGCCATGCCAGGCACAGGACAGATTGCACCCGAG | |
| TGACATTTTGAACAGAGAAAGCAACAGTGGCCCAGAAAAGGAATGTCACTTGCCTAAAGTGACACAGC | |
| ACCAAGGCTCACACCTGGAACATCACCCACGGAAACCTTAGGAGAGCCACAGGTGTTGCTGTAGTTCC | |
| TTGTCCCACCAGGTCCCTTCGTCCTTCTCAGCCCCAAATAAACACACAGATACTTATATTAATTATAA | |
| AACTGTTGGCTGATGGCTAGGGCTTCTTATTGGCCAGCTCTGTCTTAATTGACCCATTTCTATAACTC | |
| TATGTATCTCCACGTGGTCTTGGCTTACCGGAGAATGGCCGGACCTGTTACTCCTTTTGGCAGCTACA | |
| TGGTGTCTTCCCTGTGGCCCCTCTCTACCTACCTTTCCCAGAATCCTCCTCGTCTCCTAGCCCCGCCT | |
| ATCTTGCTGCCTTTATTGGCCAAGCAGTGTTTCATTCATCAACCAATAAGAGAAACACATATACAGAA | |
| AGGCATCCCCCATCACACAGGCACTTACCGGAGTTGGTCCTCCCCCATGTAGTCGATGTTCAAGGGTT | |
| TTTTCCTCTCAGACAGGATCCTGAGCTTCATCTCCCGGCCGGTCTGCCGCTTCCCACGTTTCTGCTCA | |
| GCCTGGAGGGGGAGGAATCCCAAGCCAGGGATGGGACCTGGAGGCCAAATCCACTCAGGGTCCTACAG | |
| TCATGGCCAGGGCCTCCACCACTTGCAAGGGGCCCAGGCTGCAGCCCTCCTCCCCCAGACCCAGGAAT | |
| CCAAGCTCCATGCCTCCTCCATCAGACACGGGAGTACAGGCCCACTCTTCCCTTGCACCCAGGCACCA | |
| CCAGAATGTTATCTAAAGCGACAGCTGCCCCCCCTTACCCAAACCCCAACGGGATCCAGGCTCAGTGC | |
| CCTTCCTGAGGACCAGCATTCTGCAGCTCCCCTTCCCTCCTCCTTGTAAACGCAGACACCCCCCCCCC | |
| AGGGCTAGACTCACCTTGACCAGGTAGCCCCCAAAATGAGCCCCCATGTTGGAGAGAACCTTCTTCTT | |
| CTTGGCATCGTCCTCGGCTCGCTTCTTGGCTTCCTCCTCCTCTTTGCGCATCTTCTCTTCCTGTGAAA | |
| ACAGAGGGGTTCCCTCCATGTGGCCCTACTAAGGAAGGCACGAGCCTGGGTAGTGCATGGCTAGGCTC | |
| CATAGACGGGGCCGCAGAGGGATCCACTCCTAAACTTGATATATAGTTCACCGAGCCCTCAGGTTGGC | |
| AGAAATCCTCCTGCCTCAGCTTCTCCAGGGCTGGGATCACAGGTCTGAGCCCACCGCAGGCAGCAAAG | |
| CTCAGTCATTGTGCTATGGTTTTGAGTCTCTTGATCCAGCCATGCCTGAACTTTTTAAAATTTATATG | |
| CAGTTCATCCTTTCACTAGGAAAAACAAAAAACAAAAACAAAAACAAAAAAACCTTGCGCTGTACATT | |
| ACAGGGACTTTCTGAGAACCACAGGGGACATCAACGGCAGGAAAGGAAAGTATTCGTCTCCAAGAAAG | |
| GGGCAGACACCTACCGCCAGCTTGGCCTGCCGCTCCCGCTCCTTTTCGGTTCTGAATCGCTGCTGCTC | |
| AGCTCTCTCTGCACGACGCCTCTCCTGGGGTGGGGTTGGGGATGGAAGAGAGGAAGATAGCGGAGGGG | |
| GATTGGAGGCTACCCTCTCCCCCAGTTTATCCCTCCCTCCACCAGTAGACAGCAAGAGAGGTAAGTGC | |
| GGTTTCTTTTGTTTTCTTTTCCTTTTTTGAGATAGTCTCAGAGAGCCCAGGCTGGTCTCAAATTCGTG | |
| GCAATCTACCTTCCTTGTCCCTCTAAGAGCACGCCTGGCCGAAATACGCATTGGAGTGCAACCACCCG | |
| GTGTCCCCATTGTTTCTCCCGGAGACCCCAAAGCACTTCACGGAGGAAGGGACGAGGAGGGACCGAAG | |
| GACCCGGCCTCCCCGGCCTCCCCGGCCTCCCAGGCTCACGATGCGATCTTTCAGCGCAATGAGCTCCT | |
| CTTCCTCCTTTTTGCGCTGCTCGAAGTGCACGTCAATCAGAGTCTGCAGCTCCAGTAAGTCTTTCTCC | |
| ATGCGCTTCCGGTGGATGTCCTGCAGAGGGAGCCCGTGAGGCAGAGGGACCAGACCCTAGAGCCGCCC | |
| CTCTCCGGCCCCAGGGGATGATGATTGACAGCCAGCTGGGACGGCTTCCAGCAGAGGTCAGCAAAGCA | |
| TTCCTGGCTGGCAACAGGGCACCGAGGTAAATGCAGGCATTTTCAAGAAAGGAGCAAAGAGGGGCGCC | |
| TATCAGAATGGGCTCAAGGCGCTGAGGAGCCAGCAAGTATGTCTGGGGTGGGACACCTGTCGCTTACA | |
| TCAAAGTCCACACGCTCCCCTTCTGGGATCTTGGGGGGGATCAAAGGAGGCACCACAGGACGGCTGTC | |
| GGGACAGAAATGGGAAGAGATCATTAGCAGGCTGGCCTCCTCATCCCCCCTCACCACAGACAGTTCAA | |
| AGTGACAGCTGCCCCTGAGTCTAAGCAGAGACCAGTCAGAAAGTACACCGTCTGAGCATGTTGGGTTT | |
| AATAAAACGTGTAAGACGGTGTGTTTTGCCTGTTTTGACATACTTGTAAAAAAAAAAAAAAAATGGAC | |
| AGAGCCTGATGGCACAAACCTGTCACCCGATCTACTTGGGAATCAGAGACAAGTTCCGGGCCTGCCTG | |
| ATCTACAGTAAAGCTAAAGCCATCCCTGGAAACTTGTGAGACCCGGCCTCAAACTAAAAAGTAAAAAC | |
| AGGACTGGGACTGTGGCTCAGGGGTAGAGCCCCTGCCTAGAATCCCCCAGTGAGGGGCTGGGGTGTGG | |
| CTCAGTGGTGGAGCCCCTGCCTAGAATCCCCCAGTGAGGGGCTCGGGTGTGGCTCAGTGGTAGAGCCC | |
| CTGCCTAGAATTCCCCAGTGAGGGGCTGGGGTGTGGCTCAAGACGGAGCCCTTGTGTCAAACATATAA | |
| GGCCACAGGTTGGACACTGGACATTAAAAGGG | |
| CCR5 sequence | |
| (a putative guide for insertion of a integration site is indicated) | |
| (SEQ ID NO: 3) | |
| GTAAACAGAGTCCTGTAATGCAAGGTCCGGCCTTGGCAGCCCCAGCCTGGAGCCACAGTGAG | |
| ATGTGAGCCGAGGGTTATGCTGGGAAAAACCTCTCCCTCCCAGCACCTGAAAGGCTCTGCAG | |
| GCCCAGCAGCTCAGCAAGCAAGGGTAAGGGCATGGACTAACATCTTATTTCATACTATCCCT | |
| TATAACACATCCTAATGTAATCAGCTCACAATATGAAATTATTTCATTTCTCTCCAGTCATT | |
| GTTTCAATGGGGCCTTAGGGTTGACTGGATTCTGGAGGGCCCTGCCTAGAGGAGGGGGTGCA | |
| TTCTGTCCCTATGTCCCCTCCTGCTCCATCCTCCACAGCACGTGCCTAGTGGTCTACCTTGT | |
| GGGGAATTCTTGTACCTCCCTCTTCTAGGCATGGACTAGCATTGAGAAGTGGGAGAGGAGTG | |
| TTAGGAAAAAGGGCAAATATAGACATACCTTGTCTTATTGTGCTTTACAGATATTGTTTTTG | |
| TTGTTGTTGTTGTTGTTTACAAATTGAAGGTTTGTGGCAACCCTGCCTCGAGCAAGTCTATT | |
| GGTGCTGTTTTTCCAACAGCATGTGCTTGTTTTACATCTCTGTGTCACATTTTGGTAATTCT | |
| CCCAATATTTCAAACTTTGTCATTATTTCTATATCTGTTATGGTAATCTGTGATCAGTGATC | |
| TTTGATGTCACTATTGTAGTTGTTTTGGGGCACCATGAAGTGCACCCATGTAAGATGGCAAA | |
| CAATCAATAAATGTTGTGTGTGTTCTGACTGCTCCATGGACTGCCTGTTCCTGAGACACAAT | |
| AATGTATATATAACAATTATATATATATATATTTATAACAATTATATATATATATATATATA | |
| TTTTTTTTTTGAGGCAGAGTCGCACTCTGATTGCCCAGGCTGGAGTGCAATGATGTGATTTC | |
| AGCTCACTGCAACCTCTGCCTCCCCAGGCTCAGGTGATTCTCCCACTTCAGCCTCCCAAGCT | |
| GGGACTACAGGTGTGCACCATCACACCCGGCTAATTTTTTTTTTGTATTTTTAGGAGAGACA | |
| GGGTTTTGCCATGTTGCCCAGGCTGGCCTTAAACTCCTAGACTCAAACAATCCACCTGCCTC | |
| AGCTTCCCAAAGGGCTGGGATTACAGGCATGAGCCACTGTGCCCAGCCCAAGACACAATAAT | |
| ATTGAAATTAAGCCAATTAATAACCCTACAATGGCCTCTAAGTGTTCAAGTGAAGGGAAAAG | |
| TCCCACGTCTCTCACTTTAAATCAAAATCTAGAAATGATTAAGCTTAGTAAGGAGGACATAT | |
| TGAAAGTCAAGGCCAAAAGCTCACCTCTGCACCAGTTAGCCAAATTGCGACTTCACAGGAAA | |
| AGTTCTTGAAGGATATTTAAGCTCTACTCCAGGGAACATGCAAATGAAGAGAAAACAAAGCA | |
| GCCATATTGCTAATATGGAGAAAGTTTGAGTGGTCTGGAGAAAAGATCCAACCAGCCACAAC | |
| ATTTCCTTAAGTCAAAGCCTAATCCAGAGCAAGACTCTAACTCTCTTCAATGCTATGAAGGC | |
| GGAGAGAGGTGAGGAAGCTGCAGAAGAAAAGTTTGAAGCTAGCGGAGGTTGGTTTGTGAGGT | |
| TTAATGAAAGACAACATCTCCATAACATAAAAATGCAAGATGAAGCAGCAAGTGCAAAGGGA | |
| GAAGCTGTGGCAAGTTATCCAGAAAATCTAGATAAGATAATTGATGAAAGTGTCTACACGAA | |
| ACAACAGATTTTCAGTGTAGACAAAACAGTCTTATGTTGGAAGAAGATGCCATCCAGGACTT | |
| TCACAGCTAGAGAGGAGATGTCAAGGCAAGCTGCAAAGCTCCACAGGACAGGCTGACTCTCT | |
| TTTTAGAGGTGAATGCAGCTGATGACTTTAAGTTGAAGTAAATGTTCATTTACTATTTTGTA | |
| AATCCTGGTGTCATTAAGAATTATGCGAAATCTACTCTATCTGTGCTCCATAAATGGAACAA | |
| TAAAGCCTGGATGACAACACATCTGTTTACAGCATGGTTTACTGAATATTTCAAGCCCACTA | |
| TTGAGAACTATTGCTCAGAAAAAAAGATTCCTTTCAAAATATTACTGCTCTGCACCATGTCG | |
| ATCAAGAGCTGTGTTGGAGATGTACGAGAATATTCATGTTGTTTTCATCCCTGCTAACACAA | |
| ACATCCATTCTGCAGTCCATGGACCAAGACTTTCAAGTCTTATTAAGAAATATATTTCATAA | |
| GGCTATTAAGAAATAGCTATATATATATATATAGCCTTATATAGTTTATATAGCTACCATTG | |
| ATAGTGATTCCATTGATGGATCTGAGCAAAGCAAATTGAAAAGCTTCTGGAAAGTAGTCATT | |
| ATTCTAGATGCCATTAGGAACATTTGTAATTCATGGGAGGAGGTCAAAATACCAACATTAAC | |
| AGGAGTGTGAAAGACATTGATTCCAACCCCCATAGATGACTTTCAGGGGTTCACGTCTTCAG | |
| TGGAGGAAGTCGCTGTAGATGTGGTGGAAACAGCAAGAGAACTAGAACTAGAAGTGGAGCCT | |
| GAAGTTGTGACTGAATTGCCGCACTCTCATGATCAAACTTGAACAGATGAAGAGTTGCTTCT | |
| TACATATGAGCAGTGAAAGTGGTCTCTTGAGATGGAATCTCCTCCTGGTGAAGATGCTGTGA | |
| ACACGGTTAAAATGACAACAATCGATTTAGAATATTACATAAATTTAGTTAATAAAGCAGTG | |
| GCAGGGTTTGAGAGGATTGACTCCAATTTTGAAAGAAGTGGGTAAAATGCTATCAAATAGCA | |
| TCACATGGTATGGAGAAATCTTTTGTGAAGGGAAGAGTCGACCAAGGTGGCAAATTGCATTG | |
| TCATCTTATTTTAAGAAATTGCCACAGCCACCCCCAGCTTTAGCAACCACCACCCTGATCAG | |
| TAAGCAGCCATCAACATCAAAACAAGACCGCCATCCTCTTCAGCAAAAACACTATGACTTGC | |
| TGAAGGCTCAGATGATGGTTAGCATTTTTAGCAATACAATATTTTTAATTAAGGTATGCACA | |
| TTGGTTTTTCTGACATAATACTATTGCATACTTAATAGACTACAGTATAGGATAAACACAAC | |
| TTTTATATGCACTGGGAAACCAAAAAGGTTATTTTTGAGATATTTGCTTTACTGTGGTGGTC | |
| TGAAGCTGAACTCACAATCTCACCAAGGTGTGCCTGAACCTCTTTAGCTAACTGGCCACTGC | |
| CACAGTCCACTCTGTGTTGGTCAAGATGCCCCAGAGTGGCAGGCACACTGTGTGGTCACATC | |
| CAAGGGCCTAGATATGGTGGGGGCTCCAAATGGATCTAGATATGTGAGATCTCTCTTTGATT | |
| TGACTTCTTCCAACCCACCATTTTCTGGGTGCTGGGCTCATCTCACCCAGAAAGTAGGACCC | |
| AATGTGACAGTTCCTGCCCAGTTCCCTCCTGTGGTAGCCACTTGACCCAGGGGCACTCTTGA | |
| TCCTTGCAGCCTCACTTACACACCCTATCTCTACCCCTATTAACTCTCTCCAATCCCCACTC | |
| CCCCTGCTCAGCTTGTCTGCTGCCCAGTGGGGGCCCCACCCATGCTGGCCTCTCCTTTTGCA | |
| AGTCCCCATTCCTCATATGGTTTCTTCAGAGCCCCTTTCTTTGGCTTTGAGGAGAGATGCCC | |
| TCACTCGCTTCCCCACCAATCCTGCCCACTTCTACAATCCATTCATTATCCTAATTGCCTCC | |
| GTATACAGACTGGAGTGAGAGGAGTTGATGTGATGGGTGTGGATACAGGGCTGGTGCTGTCA | |
| TCTTCTAGTAAGCCCTGGGAGAGGTGTCTGAGCCCAGGTGTCAGTGGTTTTCTTTGGAACTG | |
| TGAGTGCATAACACTTCTTTGCCTTCAGCCTTAGGCCATAGTTGCTAGTTCTGGGACAACCA | |
| GAAAAGCCCTACATAATCTCGTGTTATGTGCAGAGCTGAGTATAGAGCTCCAGGTATGATCT | |
| GACTCACTTAAGATCACAGTGAGTCTATTGTATTGTTGAACTGTTAGCTTAGACATCTGTTA | |
| CTGTACCTACATGGCACTAGCCTCACGCCTAGACACCGATCTGAAAGAAATCCCCTAAATGC | |
| ATAGAGAAGACTTCTCAGCTGAGCTAAGGGGCTCCCACCAGGTTTGAGCCTATCTAATGAAT | |
| CCATGAGGTAGACAGCCTGCACATGTCCACTTGGTTTGATGAATTGCACAAATCCCTATGGG | |
| GGATGTGGTTCATGGGCTGGGAAGTGGGTTACCCTGGGAAAGGTCTACAGGACAGAGGCAGG | |
| GATGGAGACAACAGCATGGTGAGTTCCCAACCCACCCACGATGATAGGTGTCTGAGGCAGAA | |
| GGTAAAGAGGCTGTCACCTGGTGGGTGTCATAAGACTCAAGTGTCATTGTTGAGGCACATGG | |
| GTAACAAAGCGTGGCACTGGATGGGGGTAGATTCTTCCTATTTCTGTGAGGATCAGGGGGAC | |
| TCCCTGGCTCTCCTGCTAAAGGTGGCTCTAGGGACAGGAAGAGTGTACTTCTTGACAGGGAT | |
| GTCAGAGCACTGATGGTGACAATCAGTGTGACACTGCTCACATGACTGAACAACCGAGAAGA | |
| GCCCGACTGTCTACTGAACAACGGGAAGAGCCCGACTGTCAATGACGGAGCTCTGTTAAATA | |
| TAGTTAAGGCTATTTTGTTGAATGAATGAAGCCAGACAGGAAAGAGGACAGTATCTTTAATC | |
| CATTTATAGAAGTTAAAGACAGGCTTATTTAATCTCTATGAAGACAGAGTGGCCCTTACCTC | |
| TGGGTGGAGCAAAAGGCACCTTCTGAAGTGATAGGGATGTTCCTTATCATCTTGATCCGGAG | |
| TGGTAGTTACATGCATGTGTGCATATCAAAACTCACCAAGCTGTACCACTAAGTGTGTTCTT | |
| CCTCAATAAAAATAATAAAGAACTACACTTATAAAGAATTTTTTAATAATATAGGAAAATGT | |
| CTACACTATAATCTTTAGCTAAAAAAAAAAAAAAAAGAAGCCGCCTACAGAATGGTATATGC | |
| ATGAGAACAATTAATCGAAAAGTGCATGGGAAAAGTCAGGATTGAAACATCATGTTTTAAAA | |
| GACATTGTTTTGATACTGTGAGAATGTACCTAAGTTTTTCCTTTTTTCTGTTTTTCCCAATT | |
| TTATACAATGAGCATGTGTTGGTTTTATAATTAGACATTTTGTTTGTTTGGTTTGGTTTTGA | |
| GACACAGCTTGCTGTCACCCAGGTTGGAGTGCAATGGCCCAATCTTGGTTCACTGCAACCTC | |
| CATCTCCTGGGTTCAAGAGATTCTCCCACTTCAGCCTCCTGAGTAGCTGGGACTATAGGGGC | |
| GCACCACCACATCCAGCTAATTTTGTGTATTTTTAGTAGAGATGGGGTTTCACCATGCTGGC | |
| CAGGTTGGTCTCAAACTCCTGACCTCAAGTTATCCACTCGCCTTGGCTTCCCAAAGTGCTGG | |
| GATTATAGGCATGAGCCACCGCACTTGGCCTAGACATTTGTTTTTAAAAATAAAAGATTCAT | |
| TTGCTCTTTTTACAGCCCGTCTCACTGTTGACTGATATTGACCAGGAGTCAACTCAGGCCCC | |
| AGGGATTTTCACAACAGCTGCTGTATGGCAGGGTTTCTGCTCACTGTGCTCATGTAGTTGGC | |
| CCTTGCACCCAAAGTGAATAATTAACATTCTCCCCATCCTGTTGACGATGCTCTGAAAATAT | |
| GGTCCAGAAATGGTGTGAGCAAGGAGACAGCAAAGCAATGCTTGGAACATAGGTGCAGTGAC | |
| TAGACATGGGGCAGCTGTTTAAAGACAAAAAGGCCCCAAAAAGGAGGGATGGCACGAAACAC | |
| CCTCCAATATGGGCATGGAGTCTAGAGTGACAAAGTGATCAAAAGTTCATTTCCTATGGGGT | |
| GTCCGAATGTACTTAATAATAAAAAGAGAACAAGAGCCATGCAAACTGAGAGGGACAAAGTA | |
| GAAAGAGTAGCAGACACCAAGCAACTAAGTCACAGCATGATAAGCTGCTAGCTTGTTGTCAT | |
| TATTGTATCCAGAACAACATTTCATTTAAATGCTGAAGAATTTCCCATGGGTCCCCACTTTC | |
| TTGTGAATCCTTGGGCTGAACCCCCCTGTCCTGAGTGGTTACTAGAACACACCTCTGGACCA | |
| GAAACACAAAAGTGGAGTAACGCACACTGCAAAGCTGTGCTTCCTTGTTTCAGCCTGTGAAT | |
| CCTCACCTTGTTTCCCATCTAGCCTATATTTTTCAAACTAACTTGGCCATAGAATCATGTAG | |
| TATTTAGGGTGGAAGCTGCCCCAGGTCTAGCACGTCATTTAACAGATGAGGAAATGGAAGCT | |
| TGGGCAGTGGAAGTATCTTGCCGAGGTCACACAGCAAGTCAGCAGCACAGCGTGTGTGACTC | |
| CGAGCCTGCTCCGCTAGCCCACATTGCCCTCTGGGGGTGAGTATGTCTTCACATCCTCCAAT | |
| ACCCTAATGACAGACAAACAGAACATGGCAAAGCCTCAGCTCTGCATGGTGAAAGTAAGAAC | |
| CAGCAATTGCCACAAACAGAAATACAGTGTTGGTCCGGCAGCCTCCGGGGGTTCTGCACAAG | |
| TGGATTACCAGTGAATACAAGGCTATCTATCTTTCGAAAAACCAAAGTTGTATTTATGCTAT | |
| CTATTTTCTATAAAATTTTATATTAATTTATTTGTTACCTATTTTTGAACTCTTTCAAAAGC | |
| ACACTTTATATTTCCCTGCTTAAACAGTCCCCCGAGGGTGGGTGCCCAAAAGGCTCTACACT | |
| TGTTATCATTCCCTCTCCACCACAGGCATATTGAGTAAGTTTGTATTTGGGTTTTTTTAAAA | |
| CCTCCACTCTACAGTTAAGAAAACTAAGGCACAGAGCTTCAATAATTTGGTCAGAGCCAAGT | |
| AGCAGTAATGAAGCTGGAGGTTAAACCCAGCAGCATGACTGCAGTTCTTAATCAATGCCTTT | |
| TGAATTGCACATATGGGATGAACTAGAACATTTTCTCGATGATTCGCTGTCCTTGTTATGAT | |
| TATGTTACTGAGCTCTGTTGTAGCACAGACATATGTCCCTATATGGGGCGGGGGTGGGGGTG | |
| TCTTGATCGCTGGGCTATTTCTATACTGTTCTGGCTTTTCCCAAGCAGTCATTTCTTTCTAT | |
| TCTCCAAGCACCAGCAATTAGCTTTACCTTTTCAGCTTCTAGTTTGCTGAAACTAATCTGCT | |
| ATAGACAGAGACTCCGGTGAACCAATTTTATTAGGATTTGATCAAATAAACTCTCTCTGACA | |
| AAGGACTGCTGAAAGAGTAACTAAGAGTTTGATGTTTACTGAGTGCATAGTATGTGCTAGAT | |
| GCTGGCCGTGGATGCCTCATAGAATCCTCCCAACAACTCATGAAATGACTACTGTCATTCAG | |
| CCCAATACCCAGACGAGAAAGCTGAGGGTAAGACAGGTTTCAAGCTTGGCAGTCTGACTACA | |
| GAGGCCACTGGCTTAGCCCCTGGGTTAGTCTGCCTCTGTAGGATTGGGGGCACGTAATTTTG | |
| CTGTTTGGGGTCTCATTTGCCTTCTTAGAGATCACAAGCCAAAGCTTTTTATTCTAGAGCCA | |
| AGGTCACGGAAGCCCAGAGGGCATCTTGTGGCTCGGGAGTAGCTCTCTGCTGTCTTCTCAGC | |
| TCTGCTGACAATACTTGAGATTTTCAGATGTCACCAACCGCCAAGAGAGCTTGATATGACTG | |
| TATATAGTATAGTCATAAAGAACCTGAACTTGACCATATACTTATGTCATGTGGAAAATTTC | |
| TCATAGCTTCAGATAGATTATATCTGGAGTGAAGAATCCTGCCACCTATGTATCTGGCATAG | |
| TGTGAGTCCTCATAAATGCTTACTGGTTTGAAGGGCAACAAAATAGTGAACAGAGTGAAAAT | |
| CCCCACTAAGATCCTGGGTCCAGAAAAAGATGGGAAACCTGTTTAGCTCACCCGTGAGCCCA | |
| TAGTTAAAACTCTTTAGACAACAGGTTGTTTCCGTTTACAGAGAACAATAATATTGGGTGGT | |
| GAGCATCTGTGTGGGGGTTGGGGTGGGATAGGGGATACGGGGAGAGTGGAGAAAAAGGGGAC | |
| ACAGGGTTAATGTGAAGTCCAGGATCCCCCTCTACATTTAAAGTTGGTTTAAGTTGGCTTTA | |
| ATTAATAGCAACTCTTAAGATAATCAGAATTTTCTTAACCTTTTAGCCTTACTGTTGAAAAG | |
| CCCTGTGATCTTGTACAAATCATTTGCTTCTTGGATAGTAATTTCTTTTACTAAAATGTGGG | |
| CTTTTGACTAGATGAATGTAAATGTTCTTCTAGCTCTGATATCCTTTATTCTTTATATTTTC | |
| TAACAGATTCTGTGTAGTGGGATGAGCAGAGAACAAAAACAAAATAATCCAGTGAGAAAAGC | |
| CCGTAAATAAACCTTCAGACCAGAGATCTATTCTCTAGCTTATTTTAAGCTCAACTTAAAAA | |
| GAAGAACTGTTCTCTGATTCTTTTCGCCTTCAATACACTTAATGATTTAACTCCACCCTCCT | |
| TCAAAAGAAACAGCATTTCCTACTTTTATACTGTCTATATGATTGATTTGCACAGCTCATCT | |
| GGCCAGAAGAGCTGAGACATCCGTTCCCCTACAAGAAACTCTCCCCGGTAAGTAACCTCTCA | |
| GCTGCTTGGCCTGTTAGTTAGCTTCTGAGATGAGTAAAAGACTTTACAGGAAACCCATAGAA | |
| GACATTTGGCAAACACCAAGTGCTCATACAATTATCTTAAAATATAATCTTTAAGATAAGGA | |
| AAGGGTCACAGTTTGGAATGAGTTTCAGACGGTTATAACATCAAAGATACAAAACATGATTG | |
| TGAGTGAAAGACTTTAAAGGGAGCAATAGTATTTTAATAACTAACAATCCTTACCTCTCAAA | |
| AGAAAGATTTGCAGAGAGATGAGTCTTAGCTGAAATCTTGAAATCTTATCTTCTGCTAAGGA | |
| GAACTAAACCCTCTCCAGTGAGATGCCTTCTGAATATGTGCCCACAAGAAGTTGTGTCTAAG | |
| TCTGGTTCTCTTTTTTCTTTTTCCTCCAGACAAGAGGGAAGCCTAAAAATGGTCAAAATTAA | |
| TATTAAATTACAAACGCCAAATAAAATTTTCCTCTAATATATCAGTTTCATGGCACAGTTAG | |
| TATATAATTCTTTATGGTTCAAAATTAAAAATGAGCTTTTCTAGGGGCTTCTCTCAGCTGCC | |
| TAGTCTAAGGTGCAGGGAGTTTGAGACTCACAGGGTTTAATAAGAGAAAATTCTCAGCTAGA | |
| GCAGCTGAACTTAAATAGACTAGGCAAGACAGCTGGTTATAAGACTAAACTACCCAGAATGC | |
| ATGACATTCATCTGTGGTGGCAGACGAAACATTTTTTATTATATTATTTCTTGGGTATGTAT | |
| GACAACTCTTAATTGTGGCAACTCAGAAACTACAAACACAAACTTCACAGAAAATGTGAGGA | |
| TTTTACAATTGGCTGTTGTCATCTATGACCTTCCCTGGGACTTGGGCACCCGGCCATTTCAC | |
| TCTGACTACATCATGTCACCAAACATCTGATGGTCTTGCCTTTTAATTCTCTTTTCGAGGAC | |
| TGAGAGGGAGGGTAGCATGGTAGTTAAGAGTGCAGGCTTCCCGCATTCAAAATCGGTTGCTT | |
| ACTAGCTGTGTGGCTTTGAGCAAGTTACTCACCCTCTCTGTGCTTCAAGGTCCTTGTCTGCA | |
| AAATGTGAAAAATATTTCCTGCCTCATAAGGTTGCCCTAAGGATTAAATGAATGAATGGGTA | |
| TGATGCTTAGAACAGTGATTGGCATCCAGTATGTGCCCTCGAGGCCTCTTAATTATTACTGG | |
| CTTGCTCATAGTGCATGTTCTTTGTGGGCTAACTCTAGCGTCAATAAAAATGTTAAGACTGA | |
| GTTGCAGCCGGGCATGGTGGCTCATGCCTGTAATCCCAGCATTCTAGGAGGCTGAGGCAGGA | |
| GGATCGCTTGAGCCCAGGAGTTCGAGACCAGCCTGGGCAACATAGTGTGATCTTGTATCTAT | |
| AAAAATAAACAAAATTAGCTTGGTGTGGTGGCGCCTGTAGTCCCCAGCCACTTGGAGGGGTG | |
| AGGTGAGAGGATTGCTTGAGCCCGGGATGGTCCAGGCTGCAGTGAGCCATGATCGTGCCACT | |
| GCACTCCAGCCTGGGCGACAGAGTGAGACCCTGTCTCACAACAACAACAACAACAACAAAAA | |
| GGCTGAGCTGCACCATGCTTGACCCAGTTTCTTAAAATTGTTGTCAAAGCTTCATTCACTCC | |
| ATGGTGCTATAGAGCACAAGATTTTATTTGGTGAGATGGTGCTTTCATGAATTCCCCCAACA | |
| GAGCCAAGCTCTCCATCTAGTGGACAGGGAAGCTAGCAGCAAACCTTCCCTTCACTACAAAA | |
| CTTCATTGCTTGGCCAAAAAGAGAGTTAATTCAATGTAGACATCTATGTAGGCAATTAAAAA | |
| CCTATTGATGTATAAAACAGTTTGCATTCATGGAGGGCAACTAAATACATTCTAGGACTTTA | |
| TAAAAGATCACTTTTTATTTATGCACAGGGTGGAACAAGATGGATTATCAAGTGTCAAGTCC | |
| AATCTATGACATCAATTATTATACATCGGAGCCCTGCCAAAAAATCAATGTGAAGCAAATCG | |
| CAGCCCGCCTCCTGCCTCCGCTCTACTCACTGGTGTTCATCTTTGGTTTTGTGGGCAACATG | |
| CTGGTCATCCTCATCCTGATAAACTGCAAAAGGCTGAAGAGCATGACTGACATCTACCTGCT | |
| CAACCTGGCCATCTCTGACCTGTTTTTCCTTCTTACTGTCCCCTTCTGGGCTCACTATGCTG | |
| CCGCCCAGTGGGACTTTGGAAATACAATGTGTCAACTCTTGACAGGGCTCTATTTTATAGGC | |
| TTCTTCTCTGGAATCTTCTTCATCATCCTCCTGACAATCGATAGGTACCTGGCTGTCGTCCA | |
| TGCTGTGTTTGCTTTAAAAGCCAGGACGGTCACCTTTGGGGTGGTGACAAGTGTGATCACTT | |
| GGGTGGTGGCTGTGTTTGCGTCTCTCCCAGGAATCATCTTTACCAGATCTCAAAAAGAAGGT | |
| CTTCATTACACCTGCAGCTCTCATTTTCCATACAGTCAGTATCAATTCTGGAAGAATTTCCA | |
| GACATTAAAGATAGTCATCTTGGGGCTGGTCCTGCCGCTGCTTGTCATGGTCATCTGCTACT | |
| CGGGAATCCTAAAAACTCTGCTTCGGTGTCGAAATGAGAAGAAGAGGCACAGGGCTGTGAGG | |
| CTTATCTTCACCATCATGATTGTTTATTTTCTCTTCTGGGCTCCCTACAACATTGTCCTTCT | |
| CCTGAACACCTTCCAGGAATTCTTTGGCCTGAATAATTGCAGTAGCTCTAACAGGTTGGACC | |
| AAGCTATGCAGGTGACAGAGACTCTTGGGATGACGCACTGCTGCATCAACCCCATCATCTAT | |
| GCCTTTGTCGGGGAGAAGTTCAGAAACTACCTCTTAGTCTTCTTCCAAAAGCACATTGCCAA | |
| ACGCTTCTGCAAATGCTGTTCTATTTTCCAGCAAGAGGCTCCCGAGCGAGCAAGCTCAGTTT | |
| ACACCCGATCCACTGGGGAGCAGGAAATATCTGTGGGCTTGTGACACGGACTCAAGTGGGCT | |
| GGTGACCCAGTCAGAGTTGTGCACATGGCTTAGTTTTCATACACAGCCTGGGCTGGGGGTGG | |
| GGTGGGAGAGGTCTTTTTTAAAAGGAAGTTACTGTTATAGAGGGTCTAAGATTCATCCATTT | |
| ATTTGGCATCTGTTTAAAGTAGATTAGATCTTTTAAGCCCATCAATTATAGAAAGCCAAATC | |
| AAAATATGTTGATGAAAAATAGCAACCTTTTTATCTCCCCTTCACATGCATCAAGTTATTGA | |
| CAAACTCTCCCTTCACTCCGAAAGTTCCTTATGTATATTTAAAAGAAAGCCTCAGAGAATTG | |
| CTGATTCTTGAGTTTAGTGATCTGAACAGAAATACCAAAATTATTTCAGAAATGTACAACTT | |
| TTTACCTAGTACAAGGCAACATATAGGTTGTAAATGTGTTTAAAACAGGTCTTTGTCTTGCT | |
| ATGGGGAGAAAAGACATGAATATGATTAGTAAAGAAATGACACTTTTCATGTGTGATTTCCC | |
| CTCCAAGGTATGGTTAATAAGTTTCACTGACTTAGAACCAGGCGAGAGACTTGTGGCCTGGG | |
| AGAGCTGGGGAAGCTTCTTAAATGAGAAGGAATTTGAGTTGGATCATCTATTGCTGGCAAAG | |
| ACAGAAGCCTCACTGCAAGCACTGCATGGGCAAGCTTGGCTGTAGAAGGAGACAGAGCTGGT | |
| TGGGAAGACATGGGGAGGAAGGACAAGGCTAGATCATGAAGAACCTTGACGGCATTGCTCCG | |
| TCTAAGTCATGAGCTGAGCAGGGAGATCCTGGTTGGTGTTGCAGAAGGTTTACTCTGTGGCC | |
| AAAGGAGGGTCAGGAAGGATGAGCATTTAGGGCAAGGAGACCACCAACAGCCCTCAGGTCAG | |
| GGTGAGGATGGCCTCTGCTAAGCTCAAGGCGTGAGGATGGGAAGGAGGGAGGTATTCGTAAG | |
| GATGGGAAGGAGGGAGGTATTCGTGCAGCATATGAGGATGCAGAGTCAGCAGAACTGGGGTG | |
| GATTTGGGTTGGAAGTGAGGGTCAGAGAGGAGTCAGAGAGAATCCCTAGTCTTCAAGCAGAT | |
| TGGAGAAACCCTTGAAAAGACATCAAGCACAGAAGGAGGAGGAGGAGGTTTAGGTCAAGAAG | |
| AAGATGGATTGGTGTAAAAGGATGGGTCTGGTTTGCAGAGCTTGAACACAGTCTCACCCAGA | |
| CTCCAGGCTGTCTTTCACTGAATGCTTCTGACTTCATAGATTTCCTTCCCATCCCAGCTGAA | |
| ATACTGAGGGGTCTCCAGGAGGAGACTAGATTTATGAATACACGAGGTATGAGGTCTAGGAA | |
| CATACTTCAGCTCACACATGAGATCTAGGTGAGGATTGATTACCTAGTAGTCATTTCATGGG | |
| TTGTTGGGAGGATTCTATGAGGCAACCACAGGCAGCATTTAGCACATACTACACATTCAATA | |
| AGCATCAAACTCTTAGTTACTCATTCAGGGATAGCACTGAGCAAAGCATTGAGCAAAGGGGT | |
| CCCATAGAGGTGAGGGAAGCCTGAAAAACTAAGATGCTGCCTGCCCAGTGCACACAAGTGTA | |
| GGTATCATTTTCTGCATTTAACCGTCAATAGGCAAAGGGGGGAAGGGACATATTCATTTGGA | |
| AATAAGCTGCCTTGAGCCTTAAAACCCACAAAAGTACAATTTACCAGCCTCCGTATTTCAGA | |
| CTGAATGGGGGTGGGGGGGGCGCCTTAGGTACTTATTCCAGATGCCTTCTCCAGACAAACCA | |
| GAAGCAACAGAAAAAATCGTCTCTCCCTCCCTTTGAAATGAATATACCCCTTAGTGTTTGGG | |
| TATATTCATTTCAAAGGGAGAGAGAGAGGTTTTTTTCTGTTCTGTCTCATATGATTGTGCAC | |
| ATACTTGAGACTGTTTTGAATTTGGGGGATGGCTAAAACCATCATAGTACAGGTAAGGTGAG | |
| GGAATAGTAAGTGGTGAGAACTACTCAGGGAATGAAGGTGTCAGAATAATAAGAGGTGCTAC | |
| TGACTTTCTCAGCCTCTGAATATGAACGGTGAGCATTGTGGCTGTCAGCAGGAAGCAACGAA | |
| GGGAAATGTCTTTCCTTTTGCTCTTAAGTTGTGGAGAGTGCAACAGTAGCATAGGACCCTAC | |
| CCTCTGGGCCAAGTCAAAGACATTCTGACATCTTAGTATTTGCATATTCTTATGTATGTGAA | |
| AGTTACAAATTGCTTGAAAGAAAATATGCATCTAATAAAAAACACCTTCTAAAATAATTCAT | |
| TATATTCTTGCTCTTTCAGTCAAGTGTACATTTAGAGAATAGCACATAAAACTGCCAGAGCA | |
| TTTTATAAGCAGCTGTTTTCTTCCTTAGTGTGTGTGCATGTGTGTGTGATGTATACAAAGAG | |
| AGAGATAATTGTATTTTTGTATTTTCTTTTAAATAATTTTTAAAATTGACCCTTTTCCTGAG | |
| ACAAATTGCCAGAATAGTTTGTATTTAGAGATGGTACCTCTAAGAGTAAGGTTGCTGGTTGC | |
| TGAGCAATTGACTTGAAAACTTTTAAAATTCAAATTTTAATTCCACTACTCAAAAGAATTGC | |
| CATGTTTTAAAAAAGAGAATTGGTGCCATAAGTTAGTTGTCTATGTTTGAAAATGAAGAAGA | |
| TATGCAACGTCATGGCCTGGTCACTTACCCGCAGCCCTGAGTTGTAGGCACATCATATGTGA | |
| GAATGAGGATGCTTTTCTTTCATTTAAAATCCCTCCCCAAAACTTGGCTCTAATTGCAGTCA | |
| TGACAATCATGTACATTTGGATTTATGTGCACGAGTCTCTTACCCTGAGAGAGGACAGGTGC | |
| TACAGGTGGAGGGGACCCGTCTGGGTCACGTTCACATTTTGAACATGCTGGTTTTCAGTCAC | |
| TGCACACTCATCTCCCAGCACAGGTCATGGGCAGCAGATGCAAAAGCTGCCCGTGGTCCTAT | |
| TTGGAGGTGCATGAAATGAGCAGAAGACAGAACAGCTTGATCTGACTAGAAGGGCAGCTTGT | |
| CCCTACCAAGACTTGAAGGATTGCCTTTCATCTGTTAGGGTAAAAGGTAGAATGAACCAAGG | |
| AAGGGCAGGAGGGGGCTGGGGTTAGGGTAGAAGGAAGGGGCCATGGAGAAGGGAGATCCATC | |
| CCATAGGAGGAAGGCAGTGCGGCAGGGAGGTTTGAAGGTATCAGCTTTTGTGGCTGACATAC | |
| ATGCAGTCATGTCAATTGCTCGTTTTTCCTTTTCCATCTTATTAAATGTCTTCCAACGTTAG | |
| CACGAAGAAAAGCTATTTGCAGTGTTGCCAGCCTTTCCAGAGCCCGTCCCCATTACCTCCCC | |
| AGGCCCATGCCTTTACTCCTTGGAGTTTCAACTCACGACCTTCAGGATCTGACTTTATTCAC | |
| CAACTCTGGGGTGAACGTACCTTCTGTCTCCACCCAGAGGTCTCTATCAAAGAGGAGATTGC | |
| ATGCCATGGATAAAGTCAAAGTAGAGGTGACTGTCCTTAGGAAGAGTAATGTGAAAATTCAT | |
| AAACTGGGATTCTGTTTACATTTTGTACTCCAGGGGTTCTTAGTTTAAATCGCTCTGAATAA | |
| ATTAAGATGCAATGGCATTTCAACTGTTATGATTAAATTTACAAATCATTTATTTTCTATCA | |
| CGGGGAGAGATAGAGCTCCAAATGCAAACATAACTGCTCAAGTGTTAACACTTATAATGAAA | |
| ACATAAGAATTACCACCAACTACCCTGGGGGCTAGAAGCAGAAATGTGAACCAGAAAACAAA | |
| TCATGAACTTTCCTTTTTTTTTTTGAGATGGAGTCTCGCTCTGTTGCCCAGGCTGGAGTGCA | |
| ATGGTGCGATCTCGGCTCACTGCAACCACTGCCTCCCGGGTTCAAGCAATTCTCCTGCCTCA | |
| GCCTCCTGAGTAGCTGGGACTACAGGCATGCACCACCACGCCTGGGTAATTTTTTGTATTTT | |
| TAGTAGAGACAGGGTTTCACCGTATTAGCCAGGATGCTCTCGATCTCCTGACCTCGTGATCT | |
| GCCCGCCTCGGCCTCCCACCGAAGTGCTGGGATTACAGGCATGAGCCACTGTGCCCGGCCAA | |
| CAAATCATGAACTTTCTAACTGCAGTTCCTTGTAGCTTGTTAACACATCCACTTACTTATTG | |
| TCAGAGTACGTGGAGATTTTCCACAACCCTCGGGGATAAGGCTGAACAGAAGAGGCAAAAAC | |
| GTGAAAACATTTCGATAGCTCCTATACTTTGAAATAAAATTCACTGTAAAAGTTGCTTGTAT | |
| TTTTCCAAAACAGAGTCAACCCTTAATATTTAAGATTCTGTATACAAATACATATTTTTATA | |
| TAATTAATATATATTGTCATATGACATATATCTTTATATTAATATGCATGCATATAATATAT | |
| ATTTCCTTCCTAATTTTCTATAAGCAATTTTACAAGACTGACTTCTATTTGCCTCCTTATTG | |
| TTACTACGTGGTTTGATAATCCGTTTTGTGTCATTGTGATTCTGTCATGTTTTGGGGACTTA | |
| TTTTTGTTTCTCTGGGTGGTCACTAGTTTTTTTAAAGCATTCATGGAAGAGTGTGAATCTTT | |
| TACAAGCTAGGAAGCCATGGCAAGCCTTGGGTCATACTGCCCCCGCGAGGCCACATTGGCAA | |
| ACCAGCAAGGGTGTTCAACTTCCAGACTTGGCCATGGAGAAGACACACGAGGAGGCTTTTCA | |
| CATTCAGCTCTTTAATGTTTGTCTCTGCCGGCACCATCCCAGTTGTGAAAAAGAGGTATTTC | |
| CACAGCGGCTCAGGGTAGGTAGTGCACAGCTCACATTCATCATTTCTGAAAACCGAGAGGAG | |
| TCTCCATTCGGGGTACAGGTTGATGCCTGTCGTGGAATGAAGGTTCCAACACCCAGACCAAT | |
| CTCTGCAGTGTGCTGCTCTCATGAGCTTGCAACAAGATCAGAAAATGTTTTGTGACTAAGCA | |
| TTTTTCATATTGCATAAAATGCTTCAAGCTCCTCCCTTGTTTCTCTCTATAATCCTGTATAT | |
| CTGATGATTGTGGGTACCAAGTGTTTGAAATAATCAAATGTGATTTGATGTTGGTAAATTTC | |
| TTTTTTTTTTTTTTTTTACTTCTATTTTTTTTATTATACTTTAAGTTTTAGGGTACATGTGC | |
| ACATTGTGCAGGTTAGTTACATATGTATACATGTGCCATGCTGGTGCGCTGCACCCACTAAC | |
| TCGTCATCTAGCATTAGGTATATCTCCCAATGCTATCCCTCCCCCCTCCCCCCACCCCACCA | |
| CAGTCCCCAAAGTGTGATATTCCCCTTCCTATGTCCATGTGATCTCATTGTTCAATTCCCAC | |
| CTATGAGTGAGAATATGCGGTGTTTGGTTTTTTGTTCTTGCGATAGTTTACTGAGAATGATG | |
| GTTTCCAATTTCATCCATGTCCCTACAAAGGACATGAACATAGCAAAGACTTGGAACCAACC | |
| CAAATGTCCAACAATGATAGACTGGATTAAGAAAATGTGGCACATATACACCATGGTAAATT | |
| TCTTTATCATTCGCACTCTCCTTTCTCTATTATTGTTATTGTAACTGAACCGCAGATTAGTC | |
| ACTCATTGCTTGCAGAATCCAATTAACAAGAGCGAGGTCAGATATAAAGAAAATGATTTATT | |
| CCAAACCTCCTTCAGGGAAGAGGTGCAGCCTCCTGCCTCTAAATGCACTGCTTCGCCAGGCG | |
| TGGTGGCTCACACCTGTAATCCCAGCACTTTGGGAGACCGAGGAGGGCAGATCACTTAAGGT | |
| CAGGAGTTCAAGACCGGCCTGGCCAATATAGTGAAACCCCTGCCTCTACTAAAAATACAAAA | |
| AATTAGCCAGACGTGGTGGCGGGTGCTTGTAATCCCAGCTACTCGGGAGGCTGAGGCAGGAG | |
| AATCGCTTGAACCTGGGAGGTGGAAGTTGCAGTGAGCTGACATCTAGCCACTGCACTCCAGC | |
| CTGGGTGACAGAGTGAGACTCTGTCTCAAAATAAATAAATAAATAAATAAATAAATAAATAA | |
| ATAAATAGTAAATGCACTGCTTTGCTTTTGGAGCAGAAAGCAGGCACTTTGAAAAGGCAGGG | |
| GAGGAAGTGAGCAAGGGCAGGGGGTCTGCACACTGGCATGGTGCCTGATCTATCCAGGCAGT | |
| TGAATTGGCACTTTCATAGGCAGAAATAAGTTGAAAAAGTGGCCTAAAACTCTCTAGGTGGG | |
| AGTGGATAGTGGGCATGCCTTCAACCTGCCTTTCTGGAGGGTGAGTTCCATGGCAACCCCCT | |
| GAAGGGTGAGAGTTCCATGGAGATCATGCTTTGGTCTGTAAATCAGCTGTTAACTCTCTAGA | |
| AAGTTCTGTCTTGGAGCATATAGTTAGATGAACTTGCCCTGTAAAGAATGTCTGGTGAAGGG | |
| GAAGTAAAAGGTGAGATTTGCATTTCTAAAGGGCTAAGTAGAAAGTGGGGTACAAGAGGAAA | |
| GGAGAAAAGAGAAAATAATTTAAAAAATAATTGTAACTTATTCCCTTTTACTTAGAAAAAAG | |
| GGAATACTCAGTTACATTATCACCTCGTTTACATCAAACCCTCTTATGGAATCCTATGGTTT | |
| GAAAACAAAAAGGTTGTTGAGGACCAGTGAGCCCAACCCCTTTGCTTTATAAATGAAGAGCA | |
| TTGCCTGCCCTAAGCCCCAGAGACTCTGATGTCGTGGGTCTGGAGTGGGCTCCAACAGCGGC | |
| ATGTTTTGATGGTGCTTCCCAGTGGCACGCCAGCGATGAGCCTTTGAGTAGGGAAAGTAGGA | |
| GCACTCGTGACTCCCTTCACGATCAGCACCTGTGTGCTAATAAATTCACAAAAGCCAACATA | |
| TTGGAGTCACTCAGGGAGTTTTACAAATAGTGAGGTTAAATCCAACCTCAAATAGTTCTGAT | |
| TCGATCTGCCTGCATTGCTGCCCTGTGGTTCCCCACTGTAGAAGCTCCCCAGGTGATTCTAA | |
| GTGTAGCCAAGTCTGAGAAATACTGCCTAAAGCCTGTTGGACTGACAGCAAGGGCTGTTGTC | |
| TGAGCAAGACTTTGCCTGGCCTGGGGTGGCATGTGCACCAGGAAGAGTCTCAACTTTCATAA | |
| CAGAACATTCCCCAAGCTGGTTTTTTTAAAGCATGTGAATCTAGACTTCATTGGCAATACCA | |
| AAGATCTGTATTTGAGGCTCCAAGTATTTCACTTTCATTTTTGGTTTTGGGTTATGTTTTCA | |
| CCCTTCCTTTCCAAGTGAAAAGTAAACAGAAGTGGGATGTCTGGCGCCCATGCTGAGCTTGG | |
| CAACTTCAAATTCAATAGAGAAGAAGTCTCTTGTATAGAAAAGGGCCTGTCTGAGATGTTTC | |
| TCAAATAAATATAGATTTTGCTTATGTGGCTAAAGGATTCTTCTCCCCCCATTTCCTTATCC | |
| CTGCAGTGAGCCATCCTTCTTAACTCTTTCCATGAAAGCATTATTCCTGAAGAACTGGGAAC | |
| TCATGCCAGCCCTGATCAGGCAATGATAATTCTGCAGAGAATTAGAATTTAGATTTAAATTG | |
| TCAACTCTTATACATCCTGGCATATGGTTTAAACACATGTACACACACACAAACACCTCCTA | |
| CTATTTACTGAAGAGCAGATATCTGATAACTTAATCTTTTTGGTTTTGAGTCAAGACAATTC | |
| CTCCTTTTGAAACTGCATACCGCTGAATATAATAAAATGTAATTAAGATTAAAAATAAGAAA | |
| CTAATGGGAGAATTTCAATATTGTCTATGTTCACTTTAAAATTCCTCTACTTAGGTTTACTG | |
| CCATTACCAAAGACTATTCAAAAATCCTTTTTAGGAGAATCCTAATGGTTTCCTGACATATA | |
| ATCAAATAAGGACTCTGTTGATTGGCTAACTCAATCTTCCTGTGCCAAAAAGCAGAGCCCAG | |
| CAGAGAAGAGGGCAGGGACTTGAAAGTCAGACTGACTCGAGTTCCAGCCTTGGGGCTGTGGG | |
| AGCTTGGGCAAGTGACTTAACGTCTCTGGCTCTCAGGATCTAAAAGGATTTCCAGTAGTAAT | |
| TTGGGGTGTTACTGATACAGGAGCTAAAAAGAAATTATTTAGGTGGTTAGTGAGGGTCAGAG | |
| AGTCCTCGGTAAGATTTGCCTTTTAACAAAAAGCAGCCCCAAAATCATTTGTTTGCTAACAA | |
| AGAGAAGCCTGTAAAATTGAGCTGCAGACATAGATAAGCAAGCTGGAAGCTTGCACGGGTGA | |
| ATGCCGGCAGCTGTGCCAATAGGAAAAGGCTATCTGGGGGCCAGGCATGTTCAACATGGATT | |
| CTCCATCTTCCCTTTTCTTTGTCAACCAAGTGTACAGTAAAGGAACAGGCAACATGGCACGG | |
| GCCAGGTAGAGAACCCTTCTGCATAATAAAAGATTAGGGTGAGATGGCCAGCTTCTTCCCGT | |
| GCTATGTAAATGGCATACCTGGTCCAACCAGTCTTTTGGGCCCTGTGTAAATCAGACACCGC | |
| CTCCTCAAGTTAGTCTATAAAACCCCATGCATTTTACCGTGAAACTGGGAGATCCACTCGGA | |
| ACCCCCTCCTGCACGAGAGACCTTTTCTCTTTTGCCTATTACACTTCCGCTCTTAAACTCAC | |
| TGCTCATGTGTTAGCATCCTTGATTTCCTTGGCATGAGGCAACGAACCTTGTGTATTACCCC | |
| ATACAAATGATGCTGCTTCATTACTAATAGCAACCTGACAGGGTTGTGTTGGGGTATAAATT | |
| ATCTAGACCAGGGAGATCCAATATAATTTTTTTGTAATGACGGGAATGCTTTGTATCTGCAT | |
| CATCCAAAATGGTAGCCACCAGGCCAGGGTGAAATGTGGCCAGTGTGACTGAGGAACTGAAT | |
| GTTTTCCATGATTTAATTTAAATGTGGCCAATGGCTACTGTAGGAGACAGTGTGAGTCTGGC | |
| ATATTATAAATAATAAATATTAATATAATTTGAACTTTGGCATCAGTGTTTCCTAGATTTGA | |
| ATTACTATGCAAGTTGCTTACTGTTTCCAAGCCTCAGCTTTCTAATCTGTAATTGGGGCTAA | |
| TAATAGTATCTGCCTTACAGGTTTGTTCAGAGGATAAATGAGAAATTGCATGTTGAGGGCTT | |
| AACACAGTGCCTGGCACATAAAAGCTCTGGTAACAGTTAGCCACTTTAATAATTTGCTAATA | |
| ATGGCTATTTCTTCTTCAGATTAGGATGTGCTCCCCCAAACAGTGCACTTAGACATAGCGGG | |
| CAATCCAGCTCACTCTCTGCAGTGAGAGAGAAGCACTGGCCGACCAGAGTCAGCCAGGGGCT | |
| CATGGGTATGAAATCAACAGCATGATTTTGTAAGTAATGGATGGAAAGGGCCTCACAACTTT | |
| ATGGCACTGTGTTCAATTTGCTTGGTCTTCTGTAGCTCCTTTTGAAAGCCTTTTAGGGTGGA | |
| TTAACCTGCTACCAATAATTCTGGTCAGATGTAGACTCCATAGCTCAAAGCAAACTGAGAGA | |
| GTGAGGGCAGCAGGCCAATTCCCCACCCCTTCCTTCTGGACTCTGACAGAAGCTTACACTCA | |
| AGGAAGAGCAAGTAGGAATTAACGTGTTAAGAGCTAGGTAAGCAAAACCCAATGAGAAGTTC | |
| TGGCAAAGCCCCATGGGCAGGGGTGGCTTAGGCACAGGAAACAAGTAGGATTTCATACCACG | |
| CGCCTCAGTCTACTTCCGGGGCCCTCATCCTCAGCTGTGCCTATGCAAAGGAGAGCAACCAA | |
| TAAACCCCACCGCCACTCTCCTACTGTGGAGGCCAGGGATGGCCAGGGGTAAGAGAGGGATG | |
| GGAAGTGTTTCCTCCAGCCGTCCTCTGAGAAGGAGAGGAAACTGGGCAGAGCTTCTGTCCTC | |
| CTTCAAGCAGAAACAGAAACAAAAGAAACCCCTAAGGGGGTTCTTACTTCCCCTCTAGTTCA | |
| GTTGTGCACTAACCATCTGCAGCTCAACATTCAGCATTCATTCATTGATTCAGCAAACATTG | |
| AAGGAGGGCCAGCTATGTGCCAGATGCCAACTCATGCCATGAAAGAGAGTCCCTGTCCTTAT | |
| GAAATTCACTATTTAGAGAGAAAAGCAAGCAAAAAGGCAAAGTTTGAAAAGTACTGTTGAAG | |
| TGGCATCATTGTCTGGGGTGAATACCTGAGGTTTGTGGTCTCACGCCAAGGGAATCAAGGAC | |
| TCAGGCACACAAGAAGTGAGTTTAAGAGCAGAGGTTTAATAGGCAAAAGAAAGAGAAAAAAG | |
| AATAGCTCTCTTGCCTGCACAGAGAGAGGGGCACCTGAGTGGATCTTCCTGTTTTGTGGTGA | |
| AATGCAAGGCATTTTATAGACGAGCTTGAGGAAGTGGTGTCTGATTTACTTAGGACCCGAGA | |
| GATTGGTCAGACCAGGTGTGATGTTTACATAGCATACAAAGAAGCTGGCCATCCCATCCTAA | |
| TCTTTTATTACGCAGACGGGGTCTATACCTGGCTGGTGCCATGTTGTCTGTTCCTTACTGTA | |
| CACGTGGTTGACAAAGAAAAGGGAAGATGAAGAATCCATGTTGAACATGCCTGGCCCCCAGA | |
| TAGTCTTTTCCTATTGGCACAGCTGCCGGCATTCACTCTTGCAAGCTTCCAGATTGCTTATC | |
| TATGTCTGCAGCCCAATTTTACAGGTTGCTCTTTGCTAGAAAAGAAATGATTTGGGGGCTGC | |
| TTTTCATTAA | |
| hRosa26 sequence | |
| (Putative guides for insertion of a integration site are indicated) | |
| (SEQ ID NO: 4) | |
| ACCATTTAAACCTCAAATTAAGCAACCCACAGAACCAGGAAGTTCAAGGACCATGTCTGTTT | |
| TCACCACGATGTCTTTCCCCACCCCCCCACCCCCCACTCCACCCCCCACTAAGGGCAGGGTA | |
| TTGTATCTGCCAGACTGGGTATTTGTTGAACAAGCGAGTATTTTCGCCTATTAGCTTAGTTT | |
| TTAAGGAAATCATTTTTTACTTGATTCATCATAGCTTTAATTCTATTACATACTACAATAAA | |
| AATTTGACAAGACTGATACAAATATGTAGTGGGCAATAGTTTGCCGTCTTCTTCCCTAGTAT | |
| GGTGTTTTTCAATCTGGTGACTAGAATAGGCAGTGGGCTATAAGCAGGATTCATAAGGCCTG | |
| GAGCTGAGTTATATGTGACACTGCCACCTATTCATTGTGTGACCTTGGTTTTAACCTTCAAA | |
| GTGGGTCTCCTGGACTAAAAGAATGTGAAAAGATGGGGAAATAAATCTGTAATCTGAACATG | |
| GAATGACTTAGTTACAGACCAGACATATTGTTACTGGGAATGAAAAAGTCAATATATTTGAG | |
| GGGAAAAAAATGTAAATAAATATTGAGAAAGATTTTACAAATCTAATTAGGGGAAGATAGTT | |
| ATCTCCCAATACTAGAGGGTACCAGAGTGCTTTTAAGGGGAACATTTGGTTACCCTTATTTC | |
| TTTAAAAAATGGCAGTTTAGGAAATTCTGCCCTAACTGTAGTCCCAATGTCAGATAGGACTC | |
| AGGTCTCCACTGCAAGGACCAAAATGTTAAGTTGAAGACTGAAAATGGGAAAATTTGGAAAT | |
| GTCTTTGGAACCTCAAGTACATAAAAGCCTGTAAGTGCTTCATACTCATTAACAACATAGGC | |
| ATAGAAAAAAGATATCCTTATTCTCAAGCATAGCCTTTTCTAATAAGTTCATGTTAGATGTC | |
| ATGAAGTTTAGTGAGGAGTGAAAATCTATGAGGAAAAACATGAACCATTCTACTCTGGCAAA | |
| AGTTCAGGACAAACACCATAGGCCTGTATACCAAATTTTAAACCATGTTGAATAATGTGAAA | |
| AAAAGCATCACATTGCTTATGAAAGGCTTTCCTGTCGCCCCTTAATACTTCTGTCTCAGGCT | |
| AACATGTTTGTTAATGAGTTACAGTGGTGAAGTTAAGGAAATCTGCTTCCTGTCCTAGCATG | |
| CCCATTATCCCAGCCATACAGATTTAATACCAGGAGTCACTTTAACTCCATGAAGTCATTCA | |
| ACAGGTACTTGAGTATTTACTATGTGCGTTTGTGCTAGAGTAGCCATTTCTTAAACTTTGTG | |
| GCCTCAGAGAATCCCTTCACATTCTTAAAGATTGAGGGCCCCAAATCACTTTACTAGTATCT | |
| TACCATATTAGAAAGTAAAATATTTAAATATTATAGCAACAAATGCATATTTTTAAAAAAAT | |
| AGGTGCATTGTTTTACATTTTGGCAATTGTTTCCCTAAATGTCTGACTTAAATAGAAGACAA | |
| CTGAATTGTTTCTTTTGCATTCAATTGGTTACAATGTTACTAGCAGTTTTCTATAATCTCAC | |
| GTATTAGTCATTAGGAAAATATAGCTTCACTGAGTTATGTCAATCTCCCAAATGTTGGCCCA | |
| TTTTACTGTATACTACCTAAAATATCATTGGCCGGGCACAGTGGCTTAAACCTGTAATCCTA | |
| GCACTTTGGGAGGCCAAGGTGGATCACCTGAGGTCAGGAGTTCAAGACCACCCTGGCCAACA | |
| TGGTGAAACCCCATCTCTACTAAAAATACAAAAATTAGCCGGGTACAGTGGTACACACCTGT | |
| AGTCCCAGTTATGCAGGAGGCTGAGGCAGGAGAATTGCTTGAACCCAGGAGACAGAGGTTGC | |
| AGTCAGCCAGATGTCCCAAAAAAAAAAAAAAATTATTTTCAATATCACTATCTCATGAAGTA | |
| TTGGGAAGCTGTCAAGTTCCTGATATTAGACACAAGTTTTCCCGAAATTCTGATTTTGACTC | |
| AACGTTTGGATTTTATCATTGAAAACAATTGCTGTCAGTTGTTAGGCTCCAAGGAAATAGCA | |
| GATAATTCAGCTTACATGAGTGCTTTTTCTTGAGACAACCATTTCAAAAAGTTATGTACTGT | |
| AGGGTTTAAGATTTAACAAAATGTCACTGCTTTCACAAGGACATTCTTGAGTGAAACTGGTT | |
| TTTTTCTTTTGTGGGGGTTCACACCACAAATGCATGGCAGTGAAAAATAACTTAGAGTTTGA | |
| TGCCACTGCCACAGTTTGTGCCAAGGTGCCTGAAATTTTACTTTTACCTACTGTTGCCTTAT | |
| CACCACTCTTATGTCAACATATAGTTTAGCATAAACCATGAGATTTTAAAAAGTTATTCTAC | |
| ACTTGCATTATTTCAGGACATGTGTTTGTTGCCAAGCTTTCACGTAAGAGTATCTTTAACTA | |
| GTTGGTGCTGATGCCTGGCAAATACAAGCCAAGTAACAAGTCCAGCCATGTTTATGCACGCA | |
| TCCATTGATAACGTATTAGCACAGTCAGCCCTCCATATCCTCAGGTTCTGCATCTGCAGATT | |
| CAACCAAATGTGCTTGAAAATATTTGAGAATATTTAATACAATAGTACAAATATAAGTACAC | |
| AATGTAACAACTATTTATATAGCATTCACATTGTATGAAGTATAAGAAATCTGGAAATGATT | |
| TAAAGTATATGGGAGGATGTGTGTAAGTTGTATGCAGATACAGCGCCATTTTATAAAAGGGA | |
| ATTCAACATCCTTGGGTTTTGGTGTCCTTGGCAAATGGCCAGCAAGGGTGGGGGAGTGCTGT | |
| CCAGAAACCAATCCCAAAGCAAGGGACAAATATATACTTCTACAGATGAGATATTAACTCAG | |
| AATCCATGTCTGCACACACATCTTTAATGACAAGTTGCTTTATCACTCAACAGCGGCACTAC | |
| TATGATCTTTTACTTACACTTCAGCCAGCAGAGACATTCAGCAGTGTGAGATTTTTTAAGTT | |
| TTTCAGCTATTGTGTATGTTTCTAGTGTATAATAAAGTAACTTATCCTTTAAGATACTTAAG | |
| TAGCTTTTCATTTCTAGCTTTAAAACCTGTTTTTTTTTTTTTCCCAGTAGTGGCATACCTGC | |
| ATTAAAAAATAATGCCTTACCAAAAAAAGCACTCTGAATGATTGGTTTCAAAATGATGCCAC | |
| AACATAGGTGGCACCAACACTATTCAAGATCATTCCATTCCCATCTCTAAAAAAATTTTTGG | |
| CTGGGTATGGTGGCTCACGCCTATTAACTCAACATTTTGAGAGGCCCAAAGCAAGATCACTC | |
| AGGGCTAGGAGTTGAAGACTAGCCTGAGCAACATGGCAAGATCCTGTCTCAAAATTCTTTTT | |
| AAAATTTTTTTAAAAGCCCAGGCTTGGTGGCGCATGCCTATAGTTCCAGCTACTCAGGAGGC | |
| TGAGGCAGAAGGATCTCTTGAACCCAGGAGTTTCAGGCTGTAGTTCACTATGATGGCAGCTG | |
| TGAATAGCCTGGGCAACACAGCAAGACCTCATCTCCAAAAAAAGACAAAAGAACTAAATTAT | |
| TCTACTGCAGAACATGATTAGGTAAATATCTCCAAAGCAGAAAGACAGGTTTCATATTTTCG | |
| TTAGTTTGAGTCAGTCCTTCCAAATCAAATCTTGTTTTTTATTAGTATACAGATGGTATAGC | |
| CAGTAAGTAAATGAGAAGCAGTCTTTTTAAGCCGATCCATTCTTAAATGAAAAAATATATAA | |
| ATATTTTAGAATAAATTTATTAAATTCTAAAGTTGTAGAATTTTTAAATTTGGATATTTTGG | |
| GAAAATATTTAAACCACTATTGCAAACAAAACAACAAAATGTACTTATGTTTATACTTAGGC | |
| ACAAAGAAAACTACAGTATTTTAAAGTAACCATTACACAATATTGAGGTTGCAAAGATTACT | |
| GAAGGCATAACCTAAAAAATGAGTTGATTTCTAAAAATGGGAAAAAGGAAAAAAATAATTTC | |
| TAAAAACAAGTATGCATACCTAAACCTACCTAATGACACCTTAGAAAATTCAAGTATAGCAC | |
| CATTCATTAACATCAATGAGGATGTCATCACACATCATGTAGCCTCTGCACACCGTGAGAAT | |
| AAATGAAAAAGACAGGCATCTTGCTATCATGACAATAGTTTTGACCTCGCAGACCTCTCTGT | |
| GCTTACGCAACGGATAAAGCCATAAGAACTGTCCTGCCCTCAAGGAGCAACCTAAAGTAGGA | |
| AAAAAAAAACAAAATTACACAATTATTATTTACAATTGTGAGAAGAGCTCTTGACAACATTC | |
| AAATGGAGGATACAGTGTAGTAAGGGTGAGGGTATCAAGGCTTCCTTGAGAAGTGATGTTTT | |
| GAGGCCATTCTTTCTTCTCAATAACTGGTATTTGGTTCCTGAATCCTTTAACTTCCTTACCA | |
| TTGTCACTCCTAAGCCAAATCTCATTACGTCATGTCTAGACTACTGTTAAGAGAACCACTTA | |
| AGTGGTCTCTGCAGCCCTCAATTTATTGGTGTTATCTATGGGAAAATTGCTCAAACTCTAAG | |
| CCTTAGTTTCCTACCCTATAAAATGGGGTTTTATATACAAGGAACATACTAAATACACAGGT | |
| ATACCTCAGAAACACGGCAGGCTCAATTCCAGAGCACTACAATAAAGCGAATCTCATGAATT | |
| TGTTGGTTTCCCAGTGCATAAATTATGGTTACACTATACCATAGTCTATTAAGAAGTGTACA | |
| ATAGCATTATGTATAATTGATAAATACATCACTGCTAAAAAAATGCTAACAATCTTTTTGCT | |
| GGTGGAGGGTCTTACGCCAACGTTAACGATGGCTACTGACTCATCAGGGTGGTGGTGGTTGA | |
| AGATTACGATAGCTGTGGCAATTTCTTAAAAGACAATGAAGTTTGCCACACTGACATCCTTT | |
| CACAAGACTTCTCTGTAGCATGTGATGCTGTTTGATAGCATGATAGCATTTTACCCACAGTA | |
| GAACTTTTTTTTCCTTTTCCTTTTTTTTTTTTTTTAAACGCAAGGTCTCACTCTGTCACCCA | |
| GGCTGGAGTGCAGGGGCGCCATCTCGGCTTACTGCAACCTCCTCCTCCCTGGTTCAAGAGAT | |
| TCTCCTGCCTCAGCTTCCCAAGTAGCTGGGACTACAGGTGTGCACCACACCTGGCTAATTTG | |
| GTAGAGGTGGGGTTTCACCATGTTGGCAAGGCTGGTCTTGAACTCCTGACCTCAAATGATCT | |
| ACCAGTCTCGGCCTCCTAAAGTACTGGGATTGCAGGTGTGAGCCACCACACCCAGCCAGTGG | |
| AACTTATTTCAAAATTGAAGTCAACTCTCTCACACCCTGGCACTGCTTTATCAACCAGGTTT | |
| CTGTAATTCCTAAATCCTTTATTGGCATTTTAACAATGTTCACAGCAACTTCACCAGTAGAT | |
| TCCATCTCGAGAAACCACTTTCTTTGCTCATCCCTAAAAAGCAACTCCTCATCCATTCAAAT | |
| TTGATCATGAGATTGCAGCAATTCAGTCACATCTTCAATGCTTTACTTCCAGTTCTAGTTCT | |
| CTTCCTGTTTCCACACCTGCAGTACACAAAAAGCATTCAATAACTATTACTTCATTTCTTCT | |
| ACCTATGTTTCCATTAGCTTTTGCCTATAGTACGCACTAGAGTATGTTACCATTATTTGTTA | |
| TAAGTAGTACCTCATTATTACACTATTCGTAAGCAATACCTCAAGGTCTAAGATTAGATTTT | |
| AAATCAAGGTCAGTAAAAATAGAAAAGGCTGTGAAGACTGTTGACTGACTTTACCAGAATCC | |
| ATACACTAGAGGTGAGATTAGTTAGGTGATGAAATAACCATTCTATAAACATGATCTGAAAC | |
| TCTGTTACTGTTGTCAGCAGGAAAAGCCAATGTTACATATGTTTAAAAAAGAAAAAAAAAAC | |
| CCAAAACCAGAAAACAAAAGGTGACAAAGTATCAAGACAAAAGGTCACTGATGACTGATCTC | |
| TAGGAAAAGCTGGAAAGCAGGATTATTAAATGTAACCACGACTAAGATAAAAATCAGAGACA | |
| GAAAAGTCTTTGTCACCAAGAAGATATACTCCATGAGAGAGCAGAAACAATTCATCAGGTTT | |
| AACCCTGCTCTAGATAAAATAAAACTATCTGATTCAATACTCACACTTCTCTAATAATCCAA | |
| TACATTATCCCATCTCAAGAAGAGAGAGTCACAGATAAGAAAAAAAAGGCTTCTTGAGAAGT | |
| ATGTGCTCTAATATAAACTAATATGCCACTAAGAAAGCAACCTGCAAAGTCCAGTACCAGAC | |
| TTCTGGATTTGTGACCTAACAAGGTGCTCTACAATTAACCTAACAGTCAAACCAGAGTGTTG | |
| TAAAAGAGAATTATGTAATTATGCCAAACCTCCACTCACAAAAAATATATGGAAGTAACCTA | |
| AGTTTACATTTTGCAAATCTCACACACACACTAGCCCTGACAAAAGTTTCACCAGCTTTCTC | |
| ATCCAAGTACAAGCGTGTAATATACTTAATAAATTTGTCTTATAAGGGTAAGAAATAGTATG | |
| TAACTACTTGAAAAGGAGATAGGTAGCTGGTTAATTTAAACAAAAAGCCCAAGGAAGTAAGG | |
| TGCAGGAAAAGGATAACTGCAATGATTAGTACAGGAAACCCAAAGAAGAACTGAATGGTGGG | |
| ATAGATGTACTCAGAGACCATGAGGCATCAGTTTCCTCTATGAATAGAATATTAGGAGATGT | |
| AGGTTAAATGGGACCCTGAAGTCTCTCCCAAAAAGCCTTGTTTATATGTTTTCTGAGCTTAA | |
| CTATTACTTGAGAATCAATTTCACGTATAAACCAACAAAACTAACATTTATTGAGCTTCCAG | |
| CTCTGTGCTTAGGCACTGAAAAATCACTTTCCTTAAGGATTGCAATTAAGCAGGAGAAACAC | |
| AAATAAGGTGAACTTCTCTTGTTCGAAAGAATATATTTCAACATTCCTTTTAAAAGGAAAAC | |
| CTGACCTGCAAGTTTCCAAAAATATTAATTACTATTCCTCTTTGCCTCTCAAAATTCCCATT | |
| CTGTTATTTTTTAGGAGGAGGAAAAAACAGTTCATTTGAGGAAAAATTGAGGGTCACATACT | |
| ATACAATTGAGAAGAGTTTCTCTGAAACTGTAATCATTTTTGGCAGGTAAATAGGCATATCC | |
| GAGTCAGCAAATGAACTTGAAGATACTGAGTTATACTGCCTGCCCTGTGGGGTTCCACCTTC | |
| CCCAAAAGAATTCAGAATTTTTGGGTGATCTGAGAATCTACATTAAGACAACTGTCTCCACA | |
| CACAGGAGGCCTGAAGATCGCTGACATAAGGGTCTTTTTAAAAAGTATATTTAATGGCCTAG | |
| GGCGGTGGCTCACACCTGTAATCCCAGGACTTTGGGAAGCTTAGGGCAGGAAGATCACTTGA | |
| GCCCAGGAGTTCTAACCTGTGCAGCACAGCAAAAACCCATCTCTACAAAAAAAAAAACACAA | |
| AAAAATTAGCTGGGCATGGAAGCGTGTGCCTGTAGTTCCAGCTACTCAGGAGGCTGAGGCAG | |
| GAGGATCACTTGAGCCCAGGAAGTCAAGGCTGCGTGAGCCATGATCATGCCATTGCAATCCA | |
| GTATGTGACACTAAGACTCCGTCTCAAAAAAAAAAAAAAGATAATTAAAATGTGTAAGATAC | |
| TGTATTAGCAATATAAAAAGCATTTGGTGTTAAAATGTTGGTATTATAATTCCTCAGGATAA | |
| AACTTACTTTGTGATTGTTTTCTATAACTCAAGATATGATGCTTAGAGCTCCTCCAATCAAG | |
| TGTTTCCAGGAAGTGAAAACTTGTAGGACAGAAATTTAGGCTGGGTTCATTTGTATCACACA | |
| GACCTATTCTTCATTCAAGTTCTGATATATTTAACTATGTAGCTCCTGTAACAGTTTAATGG | |
| AATCTCACCTCCCTAAAATTCATTATGCATTTTTTTTTGAAATCCAAACTCATTAACGCTTG | |
| CTTTCACTGTTGTCCAAGGCAGGCACATCTTTAAAAATGGTTTGTTGGACTTAGCTTTCAGC | |
| TAAATATATAATAAATAAAACAAAACAAGCAGTTAAATGAAATGTAATGGGCCAGAGAGCTT | |
| CAGCTTTTATTTCCTTACTGCTCAGTAAAAAGAGAAAACCATCAATGTCCACGTATTCTGTA | |
| ATCCACAGAACAAGTCCGGGGCTACAGCTATACTGTCCACAGTTGCAATTCAAATTAGATAA | |
| AAAATAAAAATTCAGTTCTTTAGTCATACCAGCCACTTTTCCAATGCTCAAGATTAATAAAA | |
| TGTCAAACCATAAAGACATTTACATGTCGCTCACTCCATTTACTTAAAGTTGGCTAGACATC | |
| AGAGTATACTAGGAGCTCAGGAGTACAAGACACTATTCCTTCAAAAAGCTCAGAATAGTTAA | |
| GGTAATTTAAATCAGCAATGACAACAACCCCAGAATTACTATGACCCACGCAGTACAAACTG | |
| CTCAGGAGTCAGAAGAAAACTGCTTTTTTAAAAGGGCAGTTTGGGTCATAGAACAACAGACC | |
| ATGGAAGGCATGACCAAAGGGGAGATGACATTTGAATCTGCAGGATTAAAAGCAGCAAGGGT | |
| AGCATTCCAAAAAGAACCACCCCACAAAGATATATGACGTCTCTATGATTTGGGTAACTGCA | |
| ATTCATTCCATGTGACTTCAGGAGAGAGGTCATATTTGTGTGTGTAGTATGTGGAAAATAGT | |
| GAAAAATGAAAAAGCTGTTAAATTGAGGAAAGTCTATCCAGGGACCTTATGCATCACATTCA | |
| CGAGAACAGAATTCATCCTGTAAACCAGGGGTGTCCAATCTTTCGGCTTCCCTGGGCCACAC | |
| TGCAAGAACTGTCTTGGGCCACATATAAAGGACAGCTGATGAGCAAAAAAAAAAAACAGACA | |
| ACAACAACAAAAAAAACACCCCGCAAAAAAAACTCCTAAAACTTTAAGAAAGTTTACGAATT | |
| TGTGTTGGGTCGCATTCAAAGCTGTCCTGGGTCCCATGCGGCCCGCGGGTTAGACAACTTGC | |
| TGTAAACAGTACAAGCCAGTAATGGAGTTTCACCTGTCATTTTCATGCTCTATCTTCCTTTA | |
| GGACAATCATCCTAACAAGATGTAAGATGGATCAAAAGATAACACTAAAGACAGAGACAGCA | |
| ATTTGGAAGCTATCACACAGGCATCTGAGATCAGTTACTAACTGGTAAGAACAGAAATGAGA | |
| GGTATTTAGAGGAAGAAAAAGGGAGATGTTGCCTAACCTCAGATCCAATTCTCTGTAAAGCA | |
| GTAGTCAAGATCACCTGGACTGTGAAGACGGTCAGGGACAGAATCCCAGCTAAGGAAAAAGG | |
| ATAAAATGAAAATCAAGATAAACATTTAAGAACGTGAACTAGGGAGGAATAAAAGCACTGCT | |
| GGGTAAGAGTCAAGCCCCAGCTCAAGCCTTAATTTGTGGTGGAACCAATCTGTCTGGTTTCG | |
| CGAGACACCAGGCTACCCAAGATCAAGAGAGGGAGAAAGCTAGTGCTATGTCTGAATACTAG | |
| AGGAGCAAGTACAACAAATGGAAAATGGGATCAAGTATGAGTGAGAGTTGCTAAGATGCCTG | |
| GTAGGGATGCAAAGGGGTAGAGAGCCTGGGGAGAGAGGGTGAGGGAGGGAAGCACTGGTTTC | |
| TCAAGCAAAAGCTAAAATTTTTCTATTAAGATTTAACCTGATGCTACACTTTGGTGGTGCAG | |
| CAAGGGTCTCAAATGGTATAAAACTCAGGTGATCATGCTTTATGTCTGTCTCTAGAAAAATG | |
| CTCCAAAAATGATAAGTAGTGATAATCCGCAGTCTCGTTGCATAAAATCAGCCCCAGGTGAA | |
| TGACTAAGCTCCATTTCCCTACCCCACCCTTATTACAATAACCTCGACACCAACTCTAGTCC | |
| GTGGGAAGATAAACTAATCGGAGTCGCCCCTCAAATCTTACAGCTGCTCACTCCCCTGCAGG | |
| GCAACGCCCAGGGACCAAGTTAGCCCCTTAAGCCTAGGCAAAAGAATCCCGCCCATAATCGA | |
| GAAGCGACTCGACATGGAGGCGATGACGAGATCACGCGAGGAGGAAAGGAGGGAGGGCTTCT | |
| TCCAGGCCCAGGGCGGTCCTTACAAGACGGGAGGCAGCAGAGAACTCCCATAAAGGTATTGC | |
| GGCACTCCCCTCCCCCTGCCCAGAAGGGTGCGGCCTTCTCTCCACCTCCTCCACCGCAGCTC | |
| CCTCAGGATTGCAGCTCGCGCCGGTTTTTGGAGAACAAGCGCCTCCCACCCACAAACCAGCC | |
| GGACCGACCCCCGCTCCTCCCCCACCCCCACGAGTGCCTGTAGCAGGTCGGGCTTGTCTCGC | |
| CCTTCAGGCGGTGGGAACCCGGGGCGGAGCCGCGGCCGCCGCCATCCAGAAGTCTCGGCCGG | |
| CAGCCCGCCCCCGCCTCCAGCGCGCGCTTCCTGCCACGTTGCGCAGGGGCGCGGGGCCAGAC | |
| ACTGCGGCGCTCGGCCTCGGGGAGGACCGTACCAACGCCCGCCTCCCCGCCACCCCCGCGCC | |
| CCGCGCAGTGGTTTCGCTCATGTGAGACTCGAGCCAGTAGCAAGGGCCCGGTCCCACAGCTT | |
| CGACAGCCAATCAGGTGTCGAAGACAAGCAGGCGGCGGGTAAACCGACTCCCCCGAAGGAAG | |
| GGGAGGGTGGGAGGACGCCCGCGCCAGAGCCGATTTCACTGACCCTCCCCTCCCGCCGCAGG | |
| AGGCCGGCCGCGCCCGCACACCCAGCATCTCTACACCCCACCTACCTACCCGCCCCACCCAG | |
| GGGGCAACGCGAGAGTCGCTAAGCGGCTGCGTACTCCCGACGGCGTAACTGACAGGAGCTTT | |
| ACTCCAACCAGAATACGCCATTTGTGTTTTCACACACGGCGGGAGGAGAAACGGCCAATCGG | |
| CGACAAGAGGCTAGCCGGAAGCGCTCCTCCCTCTGCGAGAGCAATGGCTCCGTCCGGTTTCG | |
| AGCATTTTCCGCTCCCTTCTCCCTCCCCCTCCGGTTGCCGCAGGGCGGGCCTCCCTCCCGCC | |
| TGCATCCAGCCACCCCTTTCCCTCCCAACGTAACAAACATTATGTTCCCGACTTCCCACGGG | |
| AAAGGCAACCCCCGCAAGCCACCAGACGGCCCCCCTAGCCACCCATCCCCCCAGTGTACCGC | |
| ACCTCCCCTCCCACCAGAGTTCCGCTCCCCTACCTAGCCGAGGCTCTCTGAGGAGCCGGAGC | |
| GCCGAAGCACAGCCTCTTCTCTAGGCGGCCCCGGCGGCTTCCGCTGATTGGCGGCGAGTGGG | |
| CCAATGGGTGCGGGGCGGTGGGCGGAGAGGCCAATGGCGCGGCGGGAGGGGGCGTGTCCCGG | |
| GTGCCCCTGGCGCCGGCGCTGGGAATCCCCGTGCGGTCAGTGGCGTTTCCGCTCGGGCAGCG | |
| GGCTGAGTGAGCTGCCGCCGCCGCCGCCGCCGCCGCCGCCGCCGCCGCTGCCGGGGGAGGGG | |
| CGGCCGCCGCCCGCCTGCGCTCAGAGACTCACGCAGCCCCAGTCCCGCCAGTCCGCCAACAC | |
| AGTAGTGCCGGCCCCCCTCTTTCCCTGGCCCTGCCCCCCCTCCCCGCCTTTGGCTCGCTCCG | |
| CCTTTCTGCCCCCCACCCCCACCTCACGGGTACGGGCCATTCCCGGCCAGGAAACGCCGTGG | |
| CGCCGCGTTGGGCCTAACTCGAGTCCTGCCGCCTCCCGGGAGTGCCGTGCGCCGCAGCCCGG | |
| GCCCAGGCCCCGGCAGCGCCTGGGACAAGGTAAGGGTCCGACAGAAAAGAGACCGAACCTCA | |
| CGATCGGGCCCCAGGGGAGGGAAGGGTCACCTCCTCCGTCTCCCCGCGCTCGCTCTCCTTGG | |
| GTCGTGGGCCTGGCCCTCCCCAAGCTCTTAGGAGGATGCTGCCACTTCTCACCCCCCTCGCC | |
| GCCTTGCACACACCGTTGCAACACCCCATTTTCCCAGGGAGAGAGATCCCCCTCTAATCTAG | |
| GCGACCCAACTCCCCCTTTCATGTTTTTCCTGGGTCAGGACGCTTCCCCTCCCCCAACGCCT | |
| CTTCACCCCCTTTCCTGGGAACTGCCTACTCCACGTTTACCTTTCCCTTGAGGAGAGGCCTC | |
| TTGCTGCCCTCCGCTCGAAATACACAGGCATACTTTTTTTCTCTCCCCGATCCCCCACTCCC | |
| TACCCCCGTTCTCGCGGCCTTGTGACAGACAACTCTGATCGCTCTGGGGGCCGCGATCTCCC | |
| CTCCGTAATCTTCCTGGACGCCTTCCCTCTCGTTTTCTGGCTTCCCACCTCAGATGGCTGCT | |
| TCCCAAAGGCATTACCTTCGCCACCCCCACCACACGTTCTCTGGCTCCCCGTGGCGTGTGCC | |
| ACAGCGTGTCTGAGATAGCCTCGTTGAATGTGTAGGGTTCGAGCCTGGAGTTGAGCCAGATT | |
| GTGTCGTTTTACTTGCCTTGGGCGTGGAGAACGATCTTGTGAGAATATCTTCAAAGGCAGAA | |
| AAATATTCCCTTTATGAATTCTCTTTCCCTCTGCGTGTAAGTCGGGAATGTGAAGAGGAGTG | |
| TAGGAAAGAGCCCTGGTTCAAGTAGGTAAATCGCATGAGAGGGAAAGTTAAACTGTTGGGAA | |
| AGCCCCTTCTATGCTAATTGATTCTATAGAGTCCTTGCTTGTCTCACTTCTTGGGCGTCAGT | |
| GGTCTTTCTCTTGGATATGGATGCTGCAGTCAGCTCTGCTGGTCTGGGTCAGGGGTGCGTGT | |
| ATGACCTGCATTTTCTGCTTTCTCATGTTACTTGTGCAATGTATTCACCGGTAACTCATTTC | |
| TTTCCCAGACCTCTGGGTTCCACTGGGCTTTGTCTATATTTAAGTTCATTTCTCCAGTTTCC | |
| TTCCTGCACATAGGTACTGAACGAATCCCCAAGTTCTGTGCTAATTACCTTCATCAGTTGAC | |
| TAAACAAGTTTTTAGATGACATATTTGTGACCAAGGTCATATTTACATTTCTTTGTTGGACA | |
| GATGTTACATAGCTATACTTGTGATTGGGGAGGATCCAGCTGAGTGGAGTGTGCTGAGCTTT | |
| TTAGGAGAGTGTGTACTCCCTATTTGAAATTATTTTTTGGTTGTTAATTTTATATTATTAAT | |
| GTTTTTAGGTCACAGAAAGTTCTAAGTGGTAATTTTAGATGTGTGGGATCTGAGCTAGGACT | |
| AAAGCAGAGAATACCCACGTAATCAGAGGTTTCTGGGCTCCATAGAGGACGTAGGGCTTTTT | |
| TTTTTCTATTGGATTTCTTCCAGTTTTCTCAGGATCATTAGTTCTCTTCTGTAGCCAAAAAT | |
| TCTGGCCTGTTATGGGATTAGAGTCTTTAAGGTTTACTCAGACTGTCATTATGTGTAGAAAA | |
| ATGAATTATGCCCTTTGGTAGGACATGACACAAGGCTCTGTTTCTAGCTGCAAATTTAAATT | |
| AGATTGTAGAGTGCTTGGGAAATTGGCTTTCAAAAGACCAAAGCTTAATCTTCACTCCTAAA | |
| CTGCTGGCTTAATTAAAATGGATATTTAGAATTTGGTAAATGTTGATTTTTCTAATAAAAGG | |
| CCTTGGTTTAAAAGGGTGACCTTAGGATTGTTTCTTTCTTAAAAGCATAATTCCAGCCCTTC | |
| TGGCATGGAGCACTGGTCCAAAAAAAAAAAAAAAAAGTGTGTGTAAGGAGTGGGGGTGGGGT | |
| AAAGAGAAGGTTGTTCCTTTGGGTTGGATCACAGGGGTGAGTATACAAGGCAGCAGCAGCTG | |
| CTGGCTCTGGAGCTCTGGTTGCTACGTGAGAAGCTTGAGTAGTGCTGGCTGCTGTCTCCAGG | |
| GAAGGACAGCAGTGCAGCGTCCATTAATGCTGCTGGCTGCAGGGAGCAGCACTTAGGCGATG | |
| GCTGCTTCAGGACTAAGAAGAAACCTTGCTTTTCTGGGAATTTTCACTGCTGAGCTGGTTTG | |
| CTTTTTATTGGTGGGGAGATGGGAATTAGTAATTCATAATCTCCTACCCATTTATGGATATT | |
| GGCATCTGGAAACTGGATCATGGTTAAAGCCTTTCTTTTTTTGTTTGTTTGATTTGATTTTT | |
| GTTTTTTGGCAGATTTTTGTTTTTTATCTAGACATTTGTGCTTGGATAGGACTAAAAGTTCC | |
| ATTAGAGTTTTAATTTTTCAATCAGTTTAAAAACCCAAGTAATAATTTTAAGAATCTTTCTG | |
| ATAACCACAATAGGAAGAAAATAACAGGAATTTTTTCCTGCAGCTCACATATCATGCCTTCC | |
| TCCATCTCTTTAATCATAGAATCAATTCTTATTATTTTGTTATGTGTCTCCATCCTTTCGAT | |
| TAGACCACATTTACCTTATAGACGATTTGCTAAACATTTTACTAAGCTTGAACTCTTAAACT | |
| CTAAAAAGGTGCCATTTTGGAGTGGTTTCTAAATAAATATTTTTAATTTGTATATTAGTAAT | |
| AAACTTCTCCAGATTAGATATTTTCTTTGGAGTTTGACTTATAAGATTGATTCATTATATAC | |
| ATGTTGGATATAGCCTTCTGACATCACAAATATATGTCTTTGGCCATAATCCATCTGAAATG | |
| TAGGACAGACCAGAAGAAATATGCAGAAATCGAATAAGTCTAGTTCAGGATACTGAGAAGAT | |
| GGCCTCTGAGCCCCTTAGGTGATCTCCCCTCCCCCACAACTCCTGAACATTAGGATGATCTC | |
| TGATTAAGCAAAACAGTCTGAGCGTGGAAAAACTTGAAGGAGAACCACCACCACCAATTATA | |
| TGCAATACTGGACATATTCCTGTGTGCTGTTTTTCTTCCCCAAGACTCGTGTATCCTATACT | |
| TTTTTCTCTCAGAATTTTGATTTGTTCATTTTCGTGTAAATGTACTTAAATCTCACAAACAT | |
| CTATAATTTGTAGTATCACTCTGGCATTTGTGGCAGAGAACCAAAAAGAATGGAAATGAGTT | |
| TTGTCATTCACAAATGTGGCTCACATTGTTTTCCCAGTAATAAAAGCAGACCAATGAAACAG | |
| AACCTTTAATGGATACTATTTTAGGAGGTTCCAATTCTTATTACTATCACATAGATAAGATG | |
| CAATAGCAGATAAATATGATTTCATGTATACTGGCTGTTTGACATACTTAGGGTTTAAGATA | |
| AAAATGTTTGTAGTTTTTTACTCTGTGGCTTAAGTTGCTATATAAAATAATTGCTTTTACAC | |
| TCGAATTTCCTGTTGTTTGGAACCTTTTGTGCTCTTGATATTATCATTTTTTAGAGGATCAT | |
| ACAGGCCCTTTTCATAGAAGGATTTACTTAAGTTATACCCTTGAAAACTTTTTTATATCTTT | |
| TGATACTGTTTTGTGTCCAGGAACTGACTTTCTGAAATTATTCTGGCTTTTCTGGGGAGAAT | |
| GACTATTTCATTTTTACCTTTGAATGGGGAAATAATAAAGTGCAAAGTACAGATTTGCAGAT | |
| AATTACTTTTGCTTTATCCTCTCCATGTTGAAATAACTTATGAAAAATTAGGCCATAGTTAA | |
| CAGCAGTCAATGACTATTGGATACATTTTATCAGAGGGGAACTGGATCATGAATAAAATAAA | |
| ATTTTAAAAATAATTTTTGGCTGAACTCTGGTGATTCATCAGTTTAATTTGAAGTCAGAAGG | |
| TCTAGCAGTGAATTTTATTTATAAAAATTGTATTTCAAGTGTTGAAAACTGAAACTTCTTGA | |
| CCAGTATATTTTGTTTGAGGCATCAAACTTTGCAAAATGTGCATCGTATATTTAGTGATATA | |
| ACTGGTAGTCATTTGTAATTTAAAGTATTCTTTCAAAGGCACTCTTTAGAAAGTAATGTAGT | |
| GTACCCGTGATGGGCAGGGATTGGTACCATTCCTTACTGCCAAAAATTCCAAAATATGTGGC | |
| AAAATGATTGATTTATCTTGTGGGTGGGATTCTGGGAAGTTCATGAAAGGTGGAGAGAATAT | |
| AGTTTCCTTCACTTGTCTATATACATTTTGTTAAATAAGTCTTAGGAAAACTGTTTTATTGT | |
| ATCTTTAATTATGAATTGCGTAAAAGATACCCAGTAACTTTGGGGGGAGGTGCTGTTAGAAA | |
| GCATTACATTGGAGAGAATTCCCCTACCCTGGGACAAAATGCATTCTGTCTTTAATACTTAG | |
| CGAAGGGAACTATGGGATAAAATAAACAATGAAGGTAAGCTCAGTCTGCTTTATATGTGCCC | |
| TCACTGAGCAAGGAATTTGTAATCGCATCGTGCCTCATTCGTTTATACCATCATATTGATTT | |
| TGTTTGCTGAGTACCTGAGGGAATACCTTACTTAATGTAAGGTCACATTAAGTATGTTTGAT | |
| ATGAAGACAGGGAAAGGAATTTTCTGCTTCTTGGAGTAATGTCTTAGTATTTTTAAAACACT | |
| TAAGTTTTTACATCAGGCCAGTTTTGCCTGATGCTCATGTCTGTTGCTTTGGTTGGGCTGCT | |
| GCTTTCTCTTCTGTGTTCTTATGGGTTCGTTGTGGTATAAGGATTCCCACAGCTTTCATGGC | |
| AGTATGAAGTAATGAGAAGCATTGCCTTAGCCATGTTAGTTACATGTATACTTTTGGCCTAT | |
| GTTATGAATCACAAAAAGCGGTAGCTATAGGAATGTATACAAAATAGATTTCTGTCTGGGGA | |
| ATCAAGTTTTTGATTTGTGCTACCTAATGGAGGGGAAAATGCTGAATTTCTTGCTGCTCTGT | |
| TTGAGAAATAGATGGAAGCATGGGAGGAGCCAGAGACCTCTGCAGCAGGATTTGGTCTAAGT | |
| AGAAAAGGAAGATTTTTGTTTCAAATTGCCAGCTGCTTATGTCAGACTGACTCCCTTATTAT | |
| GCCTCCAGTAGGCCTGTCAATATGGCCAAACAGCTAGATAAGTGCGGGGCAGGACAAAGGGC | |
| TCTTTGCACAGCAGGGAGGCAATGTTGGTGGGGGAGGGGCAGGAGGTAGGAAAGGCAAGAGG | |
| AGGAGGTTCTTTTCCCTGGGAGATTATTCAGTTTGGCATACAATTAAAGAAATCATTTTTAG | |
| TTCCCACTCAAGCATTGAATTTTTGCCAACCACATACTATTAACCCCAAATTTGATACATTT | |
| CAGAATATCTTGTAGGGATCCATTCTCGCCAAGGAAAAATAAAAAAATAAATAAAGCTCTGT | |
| ATAGGTTAAAATAAAATAAATCCCACACTCTGCACCCTCCTAGGTGCAAGTCACCTCCCGAG | |
| GAGACCCGTTCTAGAGCTGAATTCTCATTAAGAAATGGAAAAGAATACTCTATCTGAATAAA | |
| AACACATTGTAATACAATGTGTTTATTTGGGTTGGGATTGGACCTGAACATGTAGAATAATT | |
| TGTTTCCCTTTATGAAATAGTTGCTCGTAGTTGTCTACAATTTTATTTCATTAAGATAGGTA | |
| GCACATTACAGCTTTCATGTGTTGGGTTGCCATATGTAAAATGCTAACTGAAGAAAGGCTAC | |
| TTTTTAATTTCAGCCTCATCCTTAGTTCCTGGAGAACCTGATATTTCCTGGAGATTACTCCC | |
| TCCCCCACCTTTTAGTTTAGGCAACCTCTTTTGATACATTTGTGTTCAGCTCGCATACAAGT | |
| GGGATAGTTGCATCCAGTTTATTAAGACTTAGTATGAATCATAGAGTTGGAAAAGATCTGTT | |
| GGTTATCTGGTCCTTTAAACCAAAATCATAATGAAATATTTTGAAATTTGGGTCCCTATTGA | |
| AGTTTTCATTAAAATGTTAAAGGATCGGTGTTCTGAACAACATTTTTAGTTACTTTTAAAAT | |
| AAATGTTTTGCGTCAGTTCTTTTTTTAAAAATAAAGAATTTCATTTATAGGCAAATTAGCTG | |
| GCAATTATTTGAATTGTGATAGGATTTCTCTTTTATGAAGGAATATATGACAAGGTTTTTCA | |
| AAATGCTTAATATATTTTAAAAGACTTTAATTTTTAGAAATAATTGGTTTGAACAGTTTTCC | |
| AAGAGCACATTTGTTGCTTGGGTTGAGGTACCACCTATATTGCAATGTTACTAAACTAGCCT | |
| TAAAGTTTTCCCTTCTGTCTATACTGCATGCAACAATAAAGGGAACTGGAATGTTAATTTCC | |
| ATTTATGGATTAGCAGAGGAGATGTTTTAACCGATTAATAACCAAAAAACTGCCTTTCGTAC | |
| ACGTAATATTAAGCAAGCCTGACCAAGTTTTGTGTTATTTCTCTCTGTTAAAGAAAACTGGA | |
| TGTGTTACTACTTAACATTATATTGTTATTTAATGGTCTTGGCAGTAATGATATAATATTTC | |
| GACCAAAAGAAATTTTGAGTAATTAATTATTATTGTAATTAGTTGGAAGTTTCTCATCAGTA | |
| AAATAGCAACAGCATTAACACAAAATCTAGTGAGCTATATTTTATATTACTACAGAAATTTA | |
| GGGTAGTCATTTCTTTCTTTATAATTTATTCACATGGATTATTTCCATAAATTTGTGGGACT | |
| AAAATAGAAGCCATCTAGTCAAGCACCAGTCTCCATACCAGACAGTTTTCTCTGCATGTGCT | |
| ATGACCCACATTGCCAGTATTAAACATCCTTTACACCCTCCCCCTTCCCAGATAATTAGAAA | |
| TCTCTTCAGGGTAGCTTCCATTGCTCCTATTACCTGGATCTTGCTAGAGGCTCTAAGAAGTT | |
| CCTGGTAAAAGTGAGACAGTAAGGGACCACATTTTGATTCCAAAGGTTTTGATAACTGTTAG | |
| GGCTCCCCAAACAGCTAATCTCATTTTCACCAAGACTTAGCCAGCAGAGGGCTGGAATGGAG | |
| GTGAAACACAAGCACTGTACCTCATCTTGCCTGTGCAGCTGCTCCACCTTATTTCCTGCTAT | |
| TATTATCTCACAACGCCTCCTCCCATCAAAAAGAAACTAGGACAAAGGGGGAAAATTGGATG | |
| GGCTAATGTGATTTTTATTATGCTAGGTTGTGGGCTTGTTTATATGTACTTAAATACAAAGC | |
| TAATTTGCCCCATTCTTAAAAGTCTTTAGTGATAGAGATTTTGTAACTTCTGTATCTTCTAC | |
| TTTCTTTCTTGATAAACCATTTCAGATTCTCAGCCTTACAGAAAGAAAGGTTTTAAGCATAC | |
| TTAATTTTCGTTGGCCGTTCACAGTCATTATTACCACCAGATGCCACTGTATTATTAGCTTG | |
| AAGAAAGGTGGGCTCTCTTCTGTACATAATATCTGCAATTTGTTTTGGAAAATACTAATTTG | |
| TATAAATCTGATTTATGACTAAAATAAGGTTAAAAATTAGACCTCTATGTATGTTTACCCTA | |
| TTACCTTAGTGGGGGTGAAATTAATTAGCTCTTTGAACATAAATTTTTCATGTCTTAGAGTT | |
| CTTTTTTCAAGCTGCATAATTTATGTTCTTCAAGCCATTTTTATCCCATACCACCCCCACAA | |
| AGGGGGAAATTTTATTTTTTATCATTTTTATTGTCTTTCAATGGTGAGATTTTCGCCACCCC | |
| ACTCCTGAAATGTGAAGACTCAAATAAAACTGAGTAATCTAATAAGGTATATGCGTTGCTGA | |
| ATGTAGTAAGATGATTGTTTCATCATTCTTAGATATTATGATCTAGTTTGAATCTGGTTTCC | |
| AGTATCATGTTAGCATATTTAATACTGTTGATATGTTAATTTTAATACATGCCCAGGTGGAT | |
| CTCCTTGCTTTCTATTTGTGCCCCTTGTTTGTCGTTTTGTATGAAGGGGGTTTTTGTTGTTG | |
| GATTTTCTTCCCCATCTCTGTGTCCTGTTATGTTCTTTGGCTTATGTTTCAAAAATTCTGTT | |
| TCCTACCACCAACCTCTGTACATGCCACAACACATACAATTTGTACTTTCACAGTTTCTGTG | |
| AAGTAGGATGATCTGCAGTTAATAATCAACTGTTTGGGCATTCTTGGTATCCAAGGAAGGTT | |
| TTACTTAGAAGGAAGAACCTGGAAGGACCTGTTGGCAATTAGACTACTTCTGCGTTTATTTT | |
| ACATTTTCCCTTATTAACGTAGGCTGTTGAGAGTTGACTTGTTTTATAAGAGAAACCAGATT | |
| GACAGAGAAGACCCCCAATCAGATAGAGTTATTTTAAAAATAAATGTGTTTATTATGGTAAC | |
| ATTTGGGGTAGAATCTAAAGGGCATATTTTTAAAAAAACTTTTAGTTCTAAAGACAAAAGAG | |
| TTTAACCTAAAACAGAACAAAGAGAAGGGCCTTTGAAGCAGTATGATTGATTATAT | |
| EXAMPLE 11-CHO and Mouse Stable Site 1 Sequences-U.S. Pat. No. 7,771,997 | |
| 211> 6473 | |
| <212> DNA | |
| <213> Cricetulus griseus | |
| <400> 1 | |
| (SEQ ID NO: 5) | |
| tctagaaaca aaaccaaaaa tattaagtca ggcttggctt caggtgctgg ggtggagtgc | 60 |
| tgacaaaaat acacaaattc ctggctttct aaggcttttt cggggattca ggtattgggt | 120 |
| gatggtagaa taaaaatctg aaacataggt gatgtatctg ccatactgca tgggtgtgta | 180 |
| tgtgtgtgta tgtgtgtctg tgtgtgtgcc cagacagaaa taccatgaag gaaaaaaaca | 240 |
| cttcaaagac aggagagaag agtgacctgg gaaggactcc ccaatgagat gagaactgag | 300 |
| cacatgccag aggaggtgag gactgaacca ttcaacacaa gtggtgaata gtcctgcaga | 360 |
| cacagagagg gccagaagca ctcagaactc cagggggtca ggagtggttc tctggaggct | 420 |
| tctgcccttg gaggttcctg aggaggaggc ttccatattg aaaatgtagt tagtggccgt | 480 |
| ttccattagt acagtgacta gagagagctg agggaccact ggactgaggc ctagatgctc | 540 |
| agtcagatgg ccatgaaagc ctagacaagc acttccgggt ggaaaggaaa cagcaggtgt | 600 |
| gaggggtcag gggcaagtta gtgggagagg tcttccagat gaagtagcag gaacggagac | 660 |
| gcactggatg gccccacttg tcaaccagca aaagcttgga tcttgttcta agaggccagg | 720 |
| gacatgacaa gggtgatctc ggtttttaaa aggctttgtg ttacctaatc acttctatta | 780 |
| gtcagatact ttgtaacaca aatgagtact tcgcctgtat tttagaaact tctgggatcc | 840 |
| tgaaaaaaca caatgacatt ctggctgcaa cacctggaga ctcccagcca ggccctggac | 900 |
| ccgggtccat tcatgcaaat actcagggac agattcttca ctaggtactg atgagctgtc | 960 |
| ttggatgcaa atgtggcctc ttcattttac tacaagtcac catgagtcag gaggtgctgt | 1020 |
| ttgcacagtg tgactaagtg atggagtgtt gactgcagcc attcccggcc ccagcttgtg | 1080 |
| agagagatcc ttttaaattg aaagtaagct caaagttacc acgaagccac acatgtataa | 1140 |
| actgtgtgaa taatctgtgc acatacacaa accatgtgaa taatctgtgt acatgtataa | 1200 |
| actgtgtgaa taatctgtgt gcagcctttc cttacctact accttccagt gatcaggttt | 1260 |
| ggactgcctg tgtgctactg gaccctgaat gtccccaccg ctgtcccctg tcttttacga | 1320 |
| ttctgacatt tttaataaat tcagcggctt cccctctgct ctgtgcctag ctataccttg | 1380 |
| gtactctgca ttttggtttc tgtgacattt ctctgtgact ctgctacatt ctcagatgac | 1440 |
| atgtgacaca gaaggtgttc cctctggaga catgtgatgt ccctgtcatt agtggaatca | 1500 |
| gatgccccca aactgttgtc cagtgtttgg gaaagtgaca cgtgaaggag gatcaggaaa | 1560 |
| agaggggtgg aaatcaagat gtgtctgagt atctcatgtc cctgagtggt ccaggctgct | 1620 |
| gacttcactc ccccaagtga gggaggccat ggtgagtaca cacacctcac acatactata | 1680 |
| tccaacacac acacacacac acacacacac acgcacgcac gcacgcacgc acgcacacat | 1740 |
| gcacacacac gaactacatt tcacaaacca catacgcata ttacacccca aacgtatcac | 1800 |
| ctatacatac cacacataca cacccctcca cacatcacac acataccaca cccacacaca | 1860 |
| gcacacacat acataggcac acattcacac accacacata tacatttgtg tatgcataca | 1920 |
| tgcatacaca cacaggcaca cagacaccac acacatgcat tgtgtacgca cacatgcata | 1980 |
| cacacacata ggcacacatt gagcacacac atacatttgt gtacgcacac tacatagaca | 2040 |
| tatatgcatt tgtatatgca cacatgcatg cacacataca taggcacaca tagagcacac | 2100 |
| acatacattt gtgtatgcac acatgcacac accaatcaca tgggaagact caggttcttc | 2160 |
| actaaggttc acatgaactt agcagttcct ggttatctcg tgaaacttgg aagattgctg | 2220 |
| tggagaagag gaagcgttgg cttgagccct ggcagcaatt aaccccgccc agaagaagta | 2280 |
| ggtttaaaaa tgagagggtc tcaatgtgga acccgcaggg cgccagttca gagaagagac | 2340 |
| ctacccaagc caactgagag caaaggcaga gggatgaacc tgggatgtag tttgaacctc | 2400 |
| tgtaccagct gggcttcatg ctattttgtt atatctttat taaatattct tttagtttta | 2460 |
| tgtgcgtgaa taccttgctt gcataaatgt atgggcactg tatgtgttct tggtgccggt | 2520 |
| ggaggccagg agagggcatg gatcctccgg agctggcgtt tgagacagtt gtgacccaca | 2580 |
| gtgtggggtc tgggaactgg gtcttagtgt tccgcaagtg cagctggggc tcttaacctc | 2640 |
| tgagccatcc ctccagcttc aagaaactta ttttcttagg acatggggga agggatccag | 2700 |
| ggctttaggc ttgtttgttc agcaaatact cttttcgtgt attttgaatt ttattttatt | 2760 |
| ttactttttt gggatagaat cacattctgc agctcaggct gggcctgaac tcatcaaaat | 2820 |
| cctcctgtct cagtctacca ggtgataaga ttactgatgt gagcctggct ttgacaagca | 2880 |
| ctttagagtc cccagccctt atatattgtg tgtgtgtgtt tgtgtgtgta tgagacactt | 2940 |
| atatatatat atatatatat ctggacactt gttccaagta taatatatat atatatatat | 3000 |
| gctctaaggg tatcatatat atccttgatt tgcttttaat ttatttttta attaaaaatg | 3060 |
| attagctaca tgtcacctgt atgcgtctgt atcatctata tatccttcct tccttctctc | 3120 |
| tctttctctc ttcttcttct cacccccaag catctatttt caaatccttg tgccgaggag | 3180 |
| atgccaagag tctcgttggg ggagatggtg agggggcgat acaggggaag agcaggagga | 3240 |
| aagggggaca gactggtgtg ggtctttgga gagctcagga gaatagcagc gatcttccct | 3300 |
| gtccctggtg tcacctctta cagccaacac cattttgtgg cctggcagaa gagttgtcaa | 3360 |
| gctggtcgca ggtctgccac acaaccccaa tctggcccca agaaaaggca cctgtgtgtg | 3420 |
| actctggggt taaaggcgct gcctggtcgt ctccagctgg acttgaaact cccgtttaat | 3480 |
| aaagagttct gcaaaataat acccgcagag tcacagtgcc aggttcccgt gctttcctga | 3540 |
| agcgccaggc acgggttccc taggaaatgg ggccttgctt gccaagctcc cacggcttgc | 3600 |
| cctgcaaacg gcctgaatga tctggcactc tgcgttgcca ctgggatgaa atggaaaaaa | 3660 |
| gaaaaagaag aagtgtctct ggaagcgggc gcgctcacac aaacccgcaa cgattgtgta | 3720 |
| aacactctcc attgagaatc tggagtgcgg ttgccctcta ctggggagct gaagacagct | 3780 |
| agtgggggcg gggggaggac cgtgctagca tccttccacg gtgctcgctg gctgtggtgc | 3840 |
| atgccgggaa ccgaaacgcg gaactaaagt caagtcttgc tttggtggaa ctgacaatca | 3900 |
| acgaaatcac ttcgattgtt ttcctctttt tactggaatt cttggatttg atagatgggg | 3960 |
| gaggatcaga gggggagggg aggggcgggg agacggaggg aggaggggag gaggggagga | 4020 |
| ggggaggagg ggaggagggg aagggatgga ggaaaatact aacttttcta attcaacatg | 4080 |
| acaaagattc ggagaaagtg caccgctagt gaccgggagg aggaatgccc tattgggcat | 4140 |
| tatattccct gtcgtctaat ggaatcaaac tcttggttcc agcaccaagg attctgagcc | 4200 |
| tatcctattc aagacagtaa ctacagccca cacggaagag gctatacaac tgaagaaata | 4260 |
| aaattttcac tttatttcat ttctgtgact gcatgttcac atgtagagag ccacctgtgt | 4320 |
| ctaggggctg atgtgctggg cagtagagtt ctgagcccgt taactggaac aacccagaac | 4380 |
| tcccaccaca gttagagctt gctgagagag ggaggccctt ggtgagattt ctttgtgtat | 4440 |
| ttatttagag acagggtctc atactgtagt ccaagctagc ctccagctca cagaaattct | 4500 |
| cctgttccgg tttccaaagt actggagtta tgagtgtgtg ttaattgaac gctaagaatt | 4560 |
| tgctgattga agaaaacctc aagtgggttt ggctaatccc cacgacccca gaggctgagg | 4620 |
| caggaggaat gagagaattc aaggtttgcc agagccacag ggtgagctca atgtggagac | 4680 |
| tgtgagggtg agctcaatgt ggagactgtg agggtgagct caatgtggag actgtgaggg | 4740 |
| tgagctcaat gtggagactg tgagggtgag ctcaatgtgg agactgtgag ggtgagctca | 4800 |
| atgtggagac ctgtatcaag ataataatag tagtagtaac aatgcaggcg agggtgtggt | 4860 |
| tgagtggtag agcagttagt tgatttgaca tgcttgaggt ctcccggtcc atctgtggcc | 4920 |
| ctgcaacagg aagggaggga ggaagggggg gaacgagaga gaggaaagag agacagaagc | 4980 |
| taagataggg aatgagagag gaaggaagaa acgggaagaa attcagactc cttcctgagt | 5040 |
| tccgccaacg cctagtgaca tcctgtgcac accctaaggt ggcctttgtg tggcactggc | 5100 |
| ttgggtggtc gggaaaggca ttttcagctt gttgcagaac tgccacagta gcatgctggg | 5160 |
| tccgtgaaag tttctgcccg ttaacaagaa gtctctacta cttgtgacct caccagtgaa | 5220 |
| aatttcttta attgtctcct ggtgttctgg gttttgcatt tttgtttcta aggatacatt | 5280 |
| cctgggtgat gtcatgaagt ccccaaagac acagtggggc tgtgttggat tgggaaagat | 5340 |
| gatttatctg gggtgtcaaa aggaaaagaa gggaaacagg cacttgggaa aatgtcctcc | 5400 |
| cgcccacccg aattttggct tggcaaccgt ggtggaggag caagaaacac gtggacgttt | 5460 |
| gaggaggcat ggggtcctag gaggacagga agcagaagga gagagctggg ctgacagcct | 5520 |
| gcaggcattg cacagtttca gaaggagatt acagcatgac tgagttttta gggatccaac | 5580 |
| agggacctgg gtagagattc tgtgggctct gaggcaactt gacctcagcc agatggtatt | 5640 |
| tgaataacct gctcttagag ggaaaacaga catagcaaac agagccacgt ttagtgatga | 5700 |
| aactctcact ttgcctgagt catgtgcggc catgcccagg ggtcaggctg acactcaact | 5760 |
| caaaaacaag tgagaaattg aagacaatcc gtggtggcag ctactggaag ggccaccaca | 5820 |
| tccccagaaa gagtggagct gctaaaaagc catttgtgat aggcacagtt atcttgaatg | 5880 |
| catggagcag agattacgga aaaatcgaga atgttaatga ggcaacattc gagttgagtc | 5940 |
| attcagtgtg ggaaacccag acgcttccat cccctaaaag gaacatcttg ctctcagtca | 6000 |
| aaatggaaat aaaaattggg gcttgaattt ggcaaatgat tcagaactct gtgtaggtat | 6060 |
| tttcacacgc acagtggata attttcatgt tggagtttat ttgtgctaaa aggcagaaaa | 6120 |
| gggtaaaaag cacatcttaa gagttatgag gttctacgaa taaaaataat gttacttaca | 6180 |
| gctattcctt aattagtacc cccttccacc tgtggtaatt tcctgagata gtcagtgggg | 6240 |
| aaaagatctc tccttctctt ctttctcccc ctcccctcct ctccctccct ccctccctcc | 6300 |
| ctccctcctc tccctccctc cccctttcct tctttctttg ctccttctcc tctgcctcct | 6360 |
| tctccctttc ttcttcattt attctaagta gcttttaaca gcacaccaat tacctgtgta | 6420 |
| taacgggaaa acacaggctc aagcagctta gagaagattg atctgtgttc act | 6473 |
| <211> 7045 | |
| <212> DNA | |
| <213> Cricetulus griseus | |
| <400> 2 | |
| (SEQ ID NO: 6) | |
| actagcgtgc aattcagagg tgggtgaaga taaaaggcaa acatttgagg ccatttcctt | 60 |
| atttggcacg gcacttagga agtggaacat gcctaatcta ctggtttgta ccacctttcc | 120 |
| ctataatgga ctgtttggga agctcctggg caaccgattc tggcatctca ttggtcagag | 180 |
| gcctgttaaa tggtactctt atttgcaaag aaggctgtaa cttgtagctt taaaagcctc | 240 |
| tcctcaagaa agaagggaga aaggatatgg ctagacatat ctaatagact taaccactgt | 300 |
| gaaaagcctt agtatgaatc agatagaacc tatttttaac tcagttttga aaaaaataat | 360 |
| ctttatattt atttgtgtgt gtgtgtgtgt gtgtgtgtgt gtgtgtgtgt gtgtgtgtgt | 420 |
| gaaccacatg tagcaggtgc tggaggaggc cagaagaggg caccagatct cctggaactg | 480 |
| acaccacaca tggttatgag ctgcctgatg tgggtgctgg gaactgaact ctcgtgttct | 540 |
| gcaagagcag caactgttct cttaactgat gagccatctc tccagccccc cccataattt | 600 |
| taattgttca ttttagtaaa ttttattcat aatcaattat cacagtataa aacaatgatt | 660 |
| ttatatatat catatacata tcaaggatga cagtgagggg gatatgtgtg tgtgtgtgtg | 720 |
| tgtgtgtgtg tgtgtgtgtg tgtgttattt gtgtgtgtgc tttttaagaa ggtgccatag | 780 |
| tcactgcatt tctctgaagg atttcaaagg aatgagacat gtctgtctgc caggaaccct | 840 |
| atcttcctct ttgggaatct gacccaaatg aggtattctg aggaactgaa tgaagagctc | 900 |
| aagtagcagt gtcttaaacc caaatgtgct gtctagagaa agtcaacgtc atcagtgagc | 960 |
| tgaggagaga tttactgagc ggaagacaag cgctctttga tttaagtggc tcgaacagtc | 1020 |
| acggctgtgg agtggagcct gtgctcaggt ctgaggcagt ctttgctagc cagctgtgat | 1080 |
| gagcagtgaa gaaagggtgg agatggaggc agggtgggag cagggctatg gttcagacta | 1140 |
| ggtatcgtga gcacaccagc tggttgactt gtggtctgtg ggtcaggcgt tgtaaacgcc | 1200 |
| ctcagggtca ggcagtcaca ttgcttgaag ctgaatgggt gaggcaacac agagagtgca | 1260 |
| aagaaggcaa agtaccacct cttccccgac ccaggtcact tctgggttat agctgagact | 1320 |
| ccggacagca tgcaaccagc tggttagagc ttcagggaaa acttgatgtc tgcatgttgc | 1380 |
| tatgaaatgt gattcggtac atctggagaa aatttataat gctggctcag tcaagcactg | 1440 |
| aacaaaggta ccttggcttt gggagctaca tgacattgac ttgtaggcag actttttttt | 1500 |
| ttctgcccgc caattcccag ataaccaata tggaggctca atattaatta taaatgctcg | 1560 |
| gctgatagct caggcttgtt actagctaac tcttccaact taaatgaacc catttctatt | 1620 |
| atctacattc tcccacgtga ctttaccttg tacttcctgt ttcctctcct tgtctgactc | 1680 |
| tgcccttctg cttcccagag tccttagtct ggttctcctg cctaacctta tcctgcccag | 1740 |
| ctgctgacca agcatttata attaatatta agtctcccag tgagactctc atccagggag | 1800 |
| gacttgggtg ctcccccctc ctcattgcca tccgtgtctt cctcttccct cgcttccccc | 1860 |
| tcctcttcct gctcttcctc ctccacccct cctttcatag tattgatggc aagggtgttc | 1920 |
| tagaatggag gagtgcccat aggcatgcaa agaaaccagt taggatgctc tgtgaggggt | 1980 |
| tgtaatcata agcgatggac acaattcaag ccacagagtg aagacggaag gatgcactgt | 2040 |
| gctctagagc aacttctggg gcagaatcac agggtgagtt tctgacttga gggcgaagag | 2100 |
| gccacgagga agggagtgag tttgtctgag ctagaagcta cggcccacct cttggtagca | 2160 |
| gacctgccca caagcatgct ttgttaatca tgtgggatct gattttcctc taaatctatg | 2220 |
| ttcaactctt aagaaaatgt gaattctcac attaaaattt agatatacgt cttttggtgg | 2280 |
| ggggggtgta aaaaatcctc aagaatatgg atttctgggg gccggagaga tcgctcagag | 2340 |
| gttaagagaa ctggttgctc ttctagacat tctgagttca attcccagca accacatggt | 2400 |
| ggctcacaac catctgtaat gcgacctggt gccatcttct gacatgcatg gatacatgca | 2460 |
| ggcagaaagc tgtatacata gtaaattgat aaatcttttt ttaaaaagag tatggattct | 2520 |
| gccgggtgtt ggtggcgcac gcctttaatc ccagcactct ggaggcagag gcaggtggat | 2580 |
| ctctgtgagt tcgagaccag cctggtctat aagagctagt tccaggacag cctccaaagc | 2640 |
| cacagagaaa ccctgtctcg aaaaaccaaa aaaaaaaaaa aaaaaaaaaa aaaaaaaaga | 2700 |
| gtatggattc taagaaagcc gtaacagctg gagctgtgta cggagttcag cgtggtacta | 2760 |
| gaagaacaga cattcatgat gaaacacccc aggattttta cttagtatct agtttccatt | 2820 |
| gttgttttga gaccggctct tatgctctcc aggctggcct caaactgctg atcttcccgc | 2880 |
| ctctacctct caagtcctgg gactacttgg ctcataaaac agtttttgtc gggctccctg | 2940 |
| aagttatggt tgtacaaacc gtgggggtca atatactcac ttgggcagag agagaaggtc | 3000 |
| tgaatcccag acaatgactg catctcagga cagttgggaa gaggacaatg gcagaaggac | 3060 |
| ttagaaaaga tagactggag ggtggaaaag cagcaggaac agagaaacaa aacaggaagc | 3120 |
| ttgctatcca gggccactct ggagtcctgt ggcaagatgg aagcgggcta ggggaataca | 3180 |
| tttgtgctac tgtgtgtgtg tgtgtgtgtg tgtgtgtgtg tgtgtgtgat caatgcctat | 3240 |
| caatgttgaa ggggaaatat gtataccaca ttgattctgg gagcaattct cagtatctgg | 3300 |
| cctagagaaa ggaatggccc ctgcagaata gacagagtga atggtgccct ttatcatttg | 3360 |
| ctaaagtgaa ggagaaataa acatccttcc atagagtttc aggtaaatga accccacagt | 3420 |
| tcatctgtgc cgtggtggag gcctggccaa cagttaaaaa gattagacac ggacaaagtc | 3480 |
| tgaaggaaac acctcgaata ggaagaggag agccacctca ttctgtaact ttcctcaagg | 3540 |
| ggaagatgtt ccaagagtgg gaataaatgg tcaaaggggg gatttttaat taggaaaacg | 3600 |
| atttcctgta tcacttgtga aactggaggt tgatttgggg cataggacaa tagatttgat | 3660 |
| gctttgcaaa aagctgtttc aaagcagaga aatggaatag agacaattat gtagcgagga | 3720 |
| gggagggtgg ggcgaagatg gagacagaga agtggaagct gactttaggg aagaggaaca | 3780 |
| tagaccacag gggcggggcg gggggcaggg gcggggggcg gggctcaaag gaggcagtgg | 3840 |
| gaacgttgct agtgttcgca gcgtaagcgt gaatgtgcaa gcgtctttgt ggtgtgtgac | 3900 |
| caggagtagc gtggctggct tgtgtgctgc ttgtaatccc agtctttgag gtttccacac | 3960 |
| tgttccacag tgggtgtgat tttccctcgg agagcatgag ggctctgctt tccccacatc | 4020 |
| ctccccagcg ttcgttggta tttgtttcca agatgttagt gggtgagaca aagcctctct | 4080 |
| gttgatttgc ctttaacagg tgacaaaaaa agctcaacca ggagacattt ttgccttctt | 4140 |
| ggaaggtaat gctcccatgt agagcaatgg gacccatctc taaggtgagg ctactcttgc | 4200 |
| agtttgcacc cagctcttct gatgcaggaa ggaagttggt gggcaagcaa gactgtttgc | 4260 |
| ttcttgcgat ggacacattc tgcacacaaa ggctcaggag gggagaaggc tgtttgatgt | 4320 |
| ttagcactca ggaaggcccc tgatgcatct gtgattagct gtctccatct gtggagcaga | 4380 |
| cacggactaa ctaaaaacca gtgtttttaa attgtcaagc ctttaaggtg aggaaattga | 4440 |
| cttattgtgc tgggccatac gtagagcaag tgctctgcat tgggccaacc cccggctctg | 4500 |
| gtttctaggc accagaatgg cctagaacta actcacaatc ctcccattcc aggtctcagg | 4560 |
| tgctagaatg aaccactata ccagcctgcc tgcctgccta cctgccttcc taaattttaa | 4620 |
| atcatgggga gtaggggaga atacacttat cttagttagg gtttctattg ctgtgaagag | 4680 |
| acaccatgag catggcaact cttataaagg aaaacattta gttgggtggc agtttcagag | 4740 |
| gttttagtac attgtcatca tggctgggaa catgatggca tgcagacaga catggtgctg | 4800 |
| gagaaaggga tgagagtcct acatcttgca ggcaacagga cctcagctga gacactggct | 4860 |
| ggtaccctga gcataggaaa cctcacagcc caccctcaca gtgacatatt tccttcaaca | 4920 |
| aagccatacc tcctaatagt gccactccct atgagatgac agggccaatt acattcaaac | 4980 |
| tgctataaca ctttaaagta ttttattttt attattgtaa attatgtatg tagctgggtg | 5040 |
| gtggcagccg aggtgcacgc ctttaatccc agcacttggg aggcagaggc agatggatct | 5100 |
| ctgtgagttc aagaccagcc tggtctataa gagctagttg caaggaagga tatacaaaga | 5160 |
| acagttctag gatagccttc aaagccacag agaagtgctg tcttgaaaac caaaaattgt | 5220 |
| gctgggacct gtctctgctt tggttgcttc ccactccccc agagctggac tcttggtcaa | 5280 |
| cactgaatca gctgcaaaat aaactcctgg attcctctct tgtaacagga gcccgaagtc | 5340 |
| aggcgcccac ttgtcttctc gcaggattgc catagacttt ttctgtgtgc ccaccattcc | 5400 |
| agactgaagt agagatggca gtggcagaga ctgggaaggc tgcaacgaaa acaggaagtt | 5460 |
| attgcaccct gggaatagtc tggaaatgaa gcttcaaaac ttgcttcatg ttcagttgta | 5520 |
| cacagactca ctcccaggtt gactcacacg tgtaaatatt cctgactatg tctgcactgc | 5580 |
| ttttatctga tgcttccttc ccaaaatgcc aagtgtacaa ggtgagggaa tcacccttgg | 5640 |
| attcagagcc cagggtcgtc ctccttaacc tggacttgtc tttctccggc agcctctgac | 5700 |
| acccctcccc ccattttctc tatcagaagg tctgagcaga gttggggcac gctcatgtcc | 5760 |
| tgatacactc cttgtcttcc tgaagatcta acttctgacc cagaaagatg gctaaggtgg | 5820 |
| tgaagtgttt gacatgaaga cttggtctta agaactggag caggggaaaa aagtcggatg | 5880 |
| tggcagcatg tacccgaaat cccagaactg gggaggtaga gacggatgag tgcccggggc | 5940 |
| tagctggctg ctcagccagc ctagctgaat tgccaaattc caactcctat tgaaaaacct | 6000 |
| ttaccaaaca aacaaacaaa caaataataa caacaacaac aacaacaaac taccccatac | 6060 |
| aaggtgggcg gctcttggct cttgaggaat gactcaccca aacccaaagc ttgccacagc | 6120 |
| tgttctctgg cctaaatggg gtgggggtgg ggcagagaca gagacagaga gagacatgac | 6180 |
| ttcctgggct gggctgtgtg ctctaggcca ccaggaactt tcctgtcttg ctctctgtct | 6240 |
| ggcacagcca gagcaccagc acccagcagg tgcacacacc tccctccgtg cttcttgagc | 6300 |
| aaacacaggt gccttggtct gtctattgaa ccggagtaag ttcttgcaga tgtatgcatg | 6360 |
| gaaacaacat tgtcctggtt ttatttctac tcttgtgata aaaaccgggg aactccagga | 6420 |
| agcagctgag gcagaggcaa atgcaaggaa tgctgcctcc tagcttgctc cccatggctt | 6480 |
| gccgggcctg ctttctgcaa gcccttctct ccccattggc atgcctgaca tgaacagcgt | 6540 |
| ttgaaatgct ctcaaatgtc actttcaaag aaggcttctc tgatcttgct aactaaatca | 6600 |
| gaccatgttt caccgtgcat tatctttctg ctgtctgtct gtctgtctgt ctgtctatct | 6660 |
| gtctatcatc tatcaatcat ctatctatct atcttctatt tatctaccta tcattcaatc | 6720 |
| atctatcttc taactagtta tcatttattt atttgtttac ttactttttt tatttgagac | 6780 |
| atctatcttc taactagtta tcatttattt atttgtttac ttactttttt tatttgagac | 6780 |
| agtatttctc tgagtgacag ccttggctgt cctggaaccc attctgtaac caggctgtcc | 6840 |
| tcaaactcac agagatccaa ctgcctctgc ctctctggtg ctggggttaa agacgtgcac | 6900 |
| caccaacgcc ccgctctatc atctatttat gtacttatta ttcagtcatt atctatcctc | 6960 |
| taactatcca tcatctgtct atccatcatc tatctatcta tctatctatc tatctatcta | 7020 |
| tctatcatcc atctataatc aattg | 7045 |
| <211> 6473 | |
| <212> DNA | |
| <213> Cricetulus griseus | |
| <400> 3 | |
| (SEQ ID NO: 7) | |
| agtgaacaca gatcaatctt ctctaagctg cttgagcctg tgttttcccg ttatacacag | 60 |
| gtaattggtg tgctgttaaa agctacttag aataaatgaa gaagaaaggg agaaggaggc | 120 |
| agaggagaag gagcaaagaa agaaggaaag ggggagggag ggagaggagg gagggaggga | 180 |
| gggagggagg gagaggaggg gagggggaga aagaagagaa ggagagatct tttccccact | 240 |
| gactatctca ggaaattacc acaggtggaa gggggtacta attaaggaat agctgtaagt | 300 |
| aacattattt ttattcgtag aacctcataa ctcttaagat gtgcttttta cccttttctg | 360 |
| ccttttagca caaataaact ccaacatgaa aattatccac tgtgcgtgtg aaaataccta | 420 |
| cacagagttc tgaatcattt gccaaattca agccccaatt tttatttcca ttttgactga | 480 |
| gagcaagaty ttccttttag gggatggaag cgtctgggtt tcccacactg aatgactcaa | 540 |
| ctcgaatgtt gcctcattaa cattctcgat ttttccgtaa tctctgctcc atgcattcaa | 600 |
| gataactgtg cctatcacaa atggcttttt agcagctcca ctctttctgg ggatgtggtg | 660 |
| gcccttccag tagctgccac cacggattgt cttcaatttc tcacttgttt ttgagttgag | 720 |
| tgtcagcctg acccctgggc atggccgcac atgactcagg caaagtgaga gtttcatcac | 780 |
| taaacgtggc tctgtttgct atgtctgttt tccctctaag agcaggttat tcaaatacca | 840 |
| tctggctgag gtcaagttgc ctcagagccc acagaatctc tacccaggtc cctgttggat | 900 |
| ccctaaaaac tcagtcatgc tgtaatctcc ttctgaaact gtgcaatgcc tgcaggctgt | 960 |
| cagcccagct ctctccttct gcttcctgtc ctcctaggac cccatgcctc ctcaaacgtc | 1020 |
| cacgtgtttc ttgctcctcc accacggttg ccaagccaaa attcgggtgg gcgggaggac | 1080 |
| attttcccaa gtgcctgttt cccttctttt ccttttgaca ccccagataa atcatctttc | 1140 |
| ccaatccaac acagccccac tgtgtctttg gggacttcat gacatcaccc aggaatgtat | 1200 |
| ccttagaaac aaaaatgcaa aacccagaac accaggagac aattaaagaa attttcactg | 1260 |
| gtgaggtcac aagtagtaga gacttcttgt taacgggcag aaactttcac ggacccagca | 1320 |
| tgctactgtg gcagttctgc aacaagctga aaatgccttt cccgaccacc caagccagtg | 1380 |
| ccacacaaag gccaccttag ggtgtgcaca ggatgtcact aggcgttggc ggaactcagg | 1440 |
| aaggagtctg aatttcttcc cgtttcttcc ttcctctctc attccctatc ttagcttctg | 1500 |
| tctctctttc ctctctctcg ttccccccct tcctccctcc cttcctgttg cagggccaca | 1560 |
| gatggaccgg gagacctcaa gcatgtcaaa tcaactaact gctctaccac tcaaccacac | 1620 |
| cctcgcctgc attgttacta ctactattat tatcttgata caggtctcca cattgagctc | 1680 |
| accctcacag tctccacatt gagctcaccc tcacagtctc cacattgagc tcaccctcac | 1740 |
| agtctccaca ttgagctcac cctcacagtc tccacattga gctcaccctc acagtctcca | 1800 |
| cattgagctc accctgtggc tctggcaaac cttgaattct ctcattcctc ctgcctcagc | 1860 |
| ctctggggtc gtggggatta gccaaaccca cttgaggttt tcttcaatca gcaaattctt | 1920 |
| agcgttcaat taacacacac tcataactcc agtactttgg aaaccggaac aggagaattt | 1980 |
| ctgtgagctg gaggctagct tggactacag tatgagaccc tgtctctaaa taaatacaca | 2040 |
| aagaaatctc accaagggcc tccctctctc agcaagctct aactgtggtg ggagttctgg | 2100 |
| gttgttccag ttaacgggct cagaactcta ctgcccagca catcagcccc tagacacagg | 2160 |
| tggctctcta catgtgaaca tgcagtcaca gaaatgaaat aaagtgaaaa ttttatttct | 2220 |
| tcagttgtat agcctcttcc gtgtgggctg tagttactgt cttgaatagg ataggctcag | 2280 |
| aatccttggt gctggaacca agagtttgat tccattagac gacagggaat ataatgccca | 2340 |
| atagggcatt cctcctcccg gtcactagcg gtgcactttc tccgaatctt tgtcatgttg | 2400 |
| aattagaaaa gttagtattt tcctccatcc cttcccctcc tcccctcctc ccctcctccc | 2460 |
| ctcctcccct cctccctccg tctccccgcc cctcccctcc ccctctgatc ctcccccatc | 2520 |
| tatcaaatcc aagaattcca gtaaaaagag gaaaacaatc gaagtgattt cgttgattgt | 2580 |
| cagttccacc aaagcaagac ttgactttag ttccgcgttt cggttcccgg catgcaccac | 2640 |
| agccagcgag caccgtggaa ggatgctagc acggtcctcc ccccgccccc actagctgtc | 2700 |
| ttcagctccc cagtagaggg caaccgcact ccagattctc aatggagagt gtttacacaa | 2760 |
| tcgttgcggg tttgtgtgag cgcgcccgct tccagagaca cttcttcttt ttcttttttc | 2820 |
| catttcatcc cagtggcaac gcagagtgcc agatcattca ggccgtttgc agggcaagcc | 2880 |
| gtgggagctt ggcaagcaag gccccatttc ctagggaacc cgtgcctggc gcttcaggaa | 2940 |
| agcacgggaa cctggcactg tgactctgcg ggtattattt tgcagaactc tttattaaac | 3000 |
| gggagtttca agtccagctg gagacgacca ggcagcgcct ttaaccccag agtcacacac | 3060 |
| aggtgccttt tcttggggcc agattggggt tgtgtggcag acctgcgacc agcttgacaa | 3120 |
| ctcttctgcc aggccacaaa atggtgttgg ctgtaagagg tgacaccagg gacagggaag | 3180 |
| atcgctgcta ttctcctgag ctctccaaag acccacacca gtctgtcccc ctttcctcct | 3240 |
| gctcttcccc tgtatcgccc cctcaccatc tcccccaacg agactcttgg catctcctcg | 3300 |
| gcacaaggat ttgaaaatag atgcttgggg gtgagaagaa gaagagagaa agagagagaa | 3360 |
| ggaaggaagg atatatagat gatacagacg catacaggtg acatgtagct aatcattttt | 3420 |
| aattaaaaaa taaattaaaa gcaaatcaag gatatatatg atacccttag agcaagtgtc | 3480 |
| tcatacacac acaaacacac acacacaata tatatatata tatatatata tatatatata | 3540 |
| tatatatata ttatacttgg aacaagtgtc cagaagggct ggggactcta aagtgcttgt | 3600 |
| caaagccagg ctcacatcag taatcttatc acctggtaga ctgagacagg aggattttga | 3660 |
| tgagttcagg cccagcctga gctgcagaat gtgattctat cccaaaaaag taaaataaaa | 3720 |
| taaaattcaa aatacacgaa aagagtattt gctgaacaaa caagcctaaa gccctggatc | 3780 |
| ccttccccca tgtcctaaga aaataagttt cttgaagctg gagggatggc tcagaggtta | 3840 |
| agagccccag ctgcacttgc ggaacactaa gacccagttc ccagacccca cactgtgggt | 3900 |
| cacaactgtc tcaaacgcca gctccggagg atccatgccc tctcctggcc tccaccggca | 3960 |
| ccaagaacac atacagtgcc catacattta tgcaagcaag gtattcacgc acataaaact | 4020 |
| aaaagaatat ttaataaaga tataacaaaa tagcatgaag cccagctggt acagaggttc | 4080 |
| aaactacatc ccaggttcat ccctctgcct ttgctctcag ttggcttggg taggtctctt | 4140 |
| ctctgaactg gcgccctgcg ggttccacat tgagaccctc tcatttttaa acctacttct | 4200 |
| tctgggcggg gttaattgct gccagggctc aagccaacgc ttcctcttct ccacagcaat | 4260 |
| cttccaagtt tcacgagata accaggaact gctaagttca tgtgaacctt agtgaagaac | 4320 |
| ctgagtcttc ccatgtgatt ggtgtgtgca tgtgtgcata cacaaatgta tgtgtgtgct | 4380 |
| ctatgtgtgc ctatgtatgt gtgcatgcat gtgtgcatat acaaatgcat atatgtctat | 4440 |
| gtagtgtgcg tacacaaatg tatgtgtgtg ctcaatgtgt gcctatgtgt gtgtatgcat | 4500 |
| gtgtgcgtac acaatgcatg tgtgtggtgt ctgtgtgcct gtgtgtgtat gcatgtatgc | 4560 |
| atacacaaat gtatatgtgt ggtgtgtgaa tgtgtgccta tgtatgtgtg tgctgtgtgt | 4620 |
| gggtgtggta tgtgtgtgat gtgtggaggg gtgtgtatgt gtggtatgta taggtgatac | 4680 |
| gtttggggtg taatatgcgt atgtggtttg tgaaatgtag ttcgtgtgtg tgcatgtgtg | 4740 |
| cgtgcgtgcg tgcgtgcgtg cgtgtgtgtg tgtgtgtgtg tgtgtgtgtt ggatatagta | 4800 |
| tgtgtgaggt gtgtgtactc accatggcct ccctcacttg ggggagtgaa gtcagcagcc | 4860 |
| tggaccactc agggacatga gatactcaga cacatcttga tttccacccc tcttttcctg | 4920 |
| atcctccttc acgtgtcact ttcccaaaca ctggacaaca gtttgggggc atctgattcc | 4980 |
| actaatgaca gggacatcac atgtctccag agggaacacc ttctgtgtca catgtcatct | 5040 |
| gagaatgtag cagagtcaca gagaaatgtc acagaaacca aaatgcagag taccaaggta | 5100 |
| tagctaggca cagagcagag gggaagccgc tgaatttatt aaaaatgtca gaatcgtaaa | 5160 |
| agacagggga cagcggtggg gacattcagg gtccagtagc acacaggcag tccaaacctg | 5220 |
| atcactggaa ggtagtaggt aaggaaaggc tgcacacaga ttattcacac agtttataca | 5280 |
| tgtacacaga ttattcacat ggtttgtgta tgtgcacaga ttattcacac agtttataca | 5340 |
| tgtgtggctt cgtggtaact ttgagcttac tttcaattta aaaggatctc tctcacaagc | 5400 |
| tggggccggg aatggctgca gtcaacactc catcacttag tcacactgtg caaacagcac | 5460 |
| ctcctgactc atggtgactt gtagtaaaat gaagaggcca catttgcatc caagacagct | 5520 |
| catcagtacc tagtgaagaa tctgtccctg agtatttgca tgaatggacc cgggtccagg | 5580 |
| gcctggctgg gagtctccag gtgttgcagc cagaatgtca ttgtgttttt tcaggatccc | 5640 |
| agaagtttct aaaatacagg ccaagtactc atttgtgtta caaagtatct gactaataga | 5700 |
| agtgattagg taacacaaag ccttttaaaa accgagatca cccttgtcat gtccctggcc | 5760 |
| tcttagaaca agatccaagc ttttgctggt tgacaagtgg ggccatccag tgcgtctccg | 5820 |
| ttcctgctac ttcatctgga agacctctcc cactaacttg cccctgaccc ctcacacctg | 5880 |
| ctgtttcctt tccacccgga agtgcttgtc taggctttca tggccatctg actgagcatc | 5940 |
| taggcctcag tccagtggtc cctcagctct ctctagtcac tgtactaatg gaaacggcca | 6000 |
| ctaactacat tttcaatatg gaagcctcct cctcaggaac ctccaagggc agaagcctcc | 6060 |
| agagaaccac tcctgacccc ctggagttct gagtgcttct ggccctctct gtgtctgcag | 6120 |
| gactattcac cacttgtgtt gaatggttca gtcctcacct cctctggcat gtgctcagtt | 6180 |
| ctcatctcat tggggagtcc ttcccaggtc actcttctct cctgtctttg aagtgttttt | 6240 |
| ttccttcatg gtatttctgt ctgggcacac acacagacac acatacacac acatacacac | 6300 |
| ccatgcagta tcgcagatac atcacctatg tttcagattt ttattctacc atcacccaat | 6360 |
| acctgaatcc ccgaaaaagc cttagaaagc caggaatttg tgtatttttg tcagcactcc | 6420 |
| accccagcac ctgaagccaa gcctgactta atatttttgg ttttgtttct aga | 6473 |
| <211> 7045 | |
| <212> DNA | |
| <213> Cricetulus griseus | |
| <400> 4 | |
| (SEQ ID NO: 8) | |
| caattgatta tagatggatg atagatagat agatagatag atagatagat agatagatga | 60 |
| tggatagaca gatgatggat agttagagga tagataatga ctgaataata agtacataaa | 120 |
| tagatgatag agcggggcgt tggtggtgca cgtctttaac cccagcacca gagaggcaga | 180 |
| ggcagttgga tctctgtgag tttgaggaca gcctggttac agaatgggtt ccaggacagc | 240 |
| caaggctgtc actcagagaa atactgtctc aaataaaaaa agtaagtaaa caaataaata | 300 |
| aatgataact agttagaaga tagatgattg aatgataggt agataaatag aagatagata | 360 |
| gatagatgat tgatagatga tagacagata gacagacaga cagacagaca gacagcagaa | 420 |
| agataatgca cggtgaaaca tggtctgatt tagttagcaa gatcagagaa gccttctttg | 480 |
| aaagtgacat ttgagagcat ttcaaacgct gttcatgtca ggcatgccaa tggggagaga | 540 |
| agggcttgca gaaagcaggc ccggcaagcc atggggagca agctaggagg cagcattcct | 600 |
| tgcatttgcc tctgcctcag ctgcttcctg gagttccccg gtttttatca caacagtaga | 660 |
| aataaaacca ggacaatgtt gtttccatgc atacatctgc aagaacttac tccggttcaa | 720 |
| tagacagacc aaggcacctg tgtttgctca agaagcacgg agggaggtgt gtgcacctgc | 780 |
| tgggtgctgg tgctctggct gtgccagaca gagagcaaga caggaaagtt cctggtggcc | 840 |
| tagagcacac agcccagccc aggaagtcat gtctctctct gtctctgtct ctgccccacc | 900 |
| cccaccccat ttaggccaga gaacagctgt ggcaagcttt gggtttgggt gagtcattcc | 960 |
| tcaagagcca agagccgccc accttgtatg gggtagtttg ttgttgttgt tgttgttatt | 1020 |
| atttgtttgt ttgtttgttt ggtaaaggtt tttcaatagg agttggaatt tggcaattca | 1080 |
| gctaggctgg ctgagcagcc agctagcccc gggcactcat ccgtctctac ctccccagtt | 1140 |
| ctgggatttc gggtacatgc tcccacatcc gacttttttc ccctgctcca gttcttaaga | 1200 |
| ccaagtcttc atgtcaaaca cttcaccacc ttagccatct ttctgggtca gaagttagat | 1260 |
| cttcaggaag acaaggagtg tatcaggaca tgagcgtgcc ccaactctgc tcagaccttc | 1320 |
| tgatagagaa aatgggggga ggggtgtcag aggctgccgg agaaagacaa gtccaggtta | 1380 |
| aggaggacga ccctgggctc tgaatccaag ggtgattccc tcaccttgta cacttggcat | 1440 |
| tttgggaagg aagcatcaga taaaagcagt gcagacatag tcaggaatat ttacacgtgt | 1500 |
| gagtcaacct gggagtgagt ctgtgtacaa ctgaacatga agcaagtttt gaagcttcat | 1560 |
| ttccagacta ttcccagggt gcaataactt cctgttttcg ttgcagcctt cccagtctct | 1620 |
| gccactgcca tctctacttc agtctggaat ggtgggcaca cagaaaaagt ctatggcaat | 1680 |
| cctgcgagaa gacaagtggg cgcctgactt cgggctcctg ttacaagaga ggaatccagg | 1740 |
| agtttatttt gcagctgatt cagtgttgac caagagtcca gctctggggg agtgggaagc | 1800 |
| aaccaaagca gagacaggtc ccagcacaat ttttggtttt caagacagca cttctctgtg | 1860 |
| gctttgaagg ctatcctaga actgttcttt gtatatcctt ccttgcaact agctcttata | 1920 |
| gaccaggctg gtcttgaact cacagagatc catctgcctc tgcctcccaa gtgctgggat | 1980 |
| taaaggcgtg cacctcggct gccaccaccc agctacatac ataatttaca ataataaaaa | 2040 |
| taaaatactt taaagtgtta tagcagtttg aatgtaattg gccctgtcat ctcataggga | 2100 |
| gtggcactat taggaggtat ggctttgttg aaggaaatat gtcactgtga gggtgggctg | 2160 |
| tgaggtttcc tatgctcagg gtaccagcca gtgtctcagc tgaggtcctg ttgcctgcaa | 2220 |
| gatgtaggac tctcatccct ttctccagca ccatgtctgt ctgcatgcca tcatgttccc | 2280 |
| agccatgatg acaatgtact aaaacctctg aaactgccac ccaactaaat gttttccttt | 2340 |
| ataagagttg ccatgctcat ggtgtctctt cacagcaata gaaaccctaa ctaagataag | 2400 |
| tgtattctcc cctactcccc atgatttaaa atttaggaag gcaggtaggc aggcaggcag | 2460 |
| gctggtatag tggttcattc tagcacctga gacctggaat gggaggattg tgagttagtt | 2520 |
| ctaggccatt ctggtgccta gaaaccagag ccgggggttg gcccaatgca gagcacttgc | 2580 |
| tctacgtatg gcccagcaca ataagtcaat ttcctcacct taaaggcttg acaatttaaa | 2640 |
| aacactggtt tttagttagt ccgtgtctgc tccacagatg gagacagcta atcacagaty | 2700 |
| catcaggggc cttcctgagt gctaaacatc aaacagcctt ctcccctcct gagcctttgt | 2760 |
| gtgcagaatg tgtccatcgc aagaagcaaa cagtcttgct tgcccaccaa cttccttcct | 2820 |
| gcatcagaag agctgggtgc aaactgcaag agtagcctca ccttagagat gggtcccatt | 2880 |
| gctctacatg ggagcattac cttccaagaa ggcaaaaatg tctcctggtt gagctttttt | 2940 |
| tgtcacctgt taaaggcaaa tcaacagaga ggctttgtct cacccactaa catcttggaa | 3000 |
| acaaatacca acgaacgctg gggaggatgt ggggaaagca gagccctcat gctctccgag | 3060 |
| ggaaaatcac acccactgtg gaacagtgtg gaaacctcaa agactgggat tacaagcagc | 3120 |
| acacaagcca gccacgctac tcctggtcac acaccacaaa gacgcttgca cattcacgct | 3180 |
| tacgctgcga acactagcaa cgttcccact gcctcctttg agccccgccc cccgcccctg | 3240 |
| ccccccgccc cgcccctgtg gtctatgttc ctcttcccta aagtcagctt ccacttctct | 3300 |
| gtctccatct tcgccccacc ctccctcctc gctacataat tgtctctatt ccatttctct | 3360 |
| gctttgaaac agctttttgc aaagcatcaa atctattgtc ctatgcccca aatcaacctc | 3420 |
| cagtttcaca agtgatacag gaaatcgttt tcctaattaa aaatcccccc tttgaccatt | 3480 |
| tattcccact cttggaacat cttccccttg aggaaagtta cagaatgagg tggctctcct | 3540 |
| cttcctattc gaggtgtttc cttcagactt tgtccgtgtc taatcttttt aactgttggc | 3600 |
| caggcctcca ccacggcaca gatgaactgt ggggttcatt tacctgaaac tctatggaag | 3660 |
| gatgtttatt tctccttcac tttagcaaat gataaagggc accattcact ctgtctattc | 3720 |
| tgcaggggcc attcctttct ctaggccaga tactgagaat tgctcccaga atcaatgtgg | 3780 |
| tatacatatt tccccttcaa cattgatagg cattgatcac acacacacac acacacacac | 3840 |
| acacacacac acacagtagc acaaatgtat tcccctagcc cgcttccatc ttgccacagg | 3900 |
| actccagagt ggccctggat agcaagcttc ctgttttgtt tctctgttcc tgctgctttt | 3960 |
| ccaccctcca gtctatcttt tctaagtcct tctgccattg tcctcttccc aactgtcctg | 4020 |
| agatgcagtc attgtctggg attcagacct tctctctctg cccaagtgag tatattgacc | 4080 |
| cccacggttt gtacaaccat aacttcaggg agcccgacaa aaactgtttt atgagccaag | 4140 |
| tagtcccagg acttgagagg tagaggcggg aagatcagca gtttgaggcc agcctggaga | 4200 |
| gcataagagc cggtctcaaa acaacaatgg aaactagata ctaagtaaaa atcctggggt | 4260 |
| gtttcatcat gaatgtctgt tcttctagta ccacgctgaa ctccgtacac agctccagct | 4320 |
| gttacggctt tcttagaatc catactcttt tttttttttt tttttttttt ttttttttgg | 4380 |
| tttttcgaga cagggtttct ctgtggcttt ggaggctgtc ctggaactag ctcttataga | 4440 |
| ccaggctggt ctcgaactca cagagatcca cctgcctctg cctccagagt gctgggatta | 4500 |
| aaggcgtgcg ccaccaacac ccggcagaat ccatactctt tttaaaaaaa gatttatcaa | 4560 |
| tttactatgt atacagcttt ctgcctgcat gtatccatgc atgtcagaag atggcaccag | 4620 |
| gtcgcattac agatggttgt gagccaccat gtggttgctg ggaattgaac tcagaatgtc | 4680 |
| tagaagagca accagttctc ttaacctctg agccatctct ccggccccca gaaatccata | 4740 |
| ttcttgagga ttttttacac cccccccacc aaaagacgta tatctaaatt ttaatgtgag | 4800 |
| aattcacatt ttcttaagag ttgaacatag atttagagga aaatcagatc ccacatgatt | 4860 |
| aacaaagcat gcttgtgggc aggtctgcta ccaagaggtg ggccgtagct tctagctcag | 4920 |
| acaaactcac tcccttcctc gtggcctctt cgccctcaag tcagaaactc accctgtgat | 4980 |
| tctgccccag aagttgctct agagcacagt gcatccttcc gtcttcactc tgtggcttga | 5040 |
| attgtgtcca tcgcttatga ttacaacccc tcacagagca tcctaactgg tttctttgca | 5100 |
| tgcctatggg cactcctcca ttctagaaca cccttgccat caatactatg aaaggagggg | 5160 |
| tggaggagga agagcaggaa gaggaggggg aagcgaggga agaggaagac acggatggca | 5220 |
| atgaggaggg gggagcaccc aagtcctccc tggatgagag tctcactggg agacttaata | 5280 |
| ttaattataa atgcttggtc agcagctggg caggataagg ttaggcagga gaaccagact | 5340 |
| aaggactctg ggaagcagaa gggcagagtc agacaaggag aggaaacagg aagtacaagg | 5400 |
| taaagtcacg tcgcagaatg tagataatag aaatgggttc atttaagttg gaagagttag | 5460 |
| ctagtaacaa gcctgagcta tcagccgagc atttataatt aatattgagc ctccatattg | 5520 |
| gttatctggg aattggcggg cagaaaaaaa aaagtctgcc tacaagtcaa tgtcatgtag | 5580 |
| ctcccaaagc caaggtacct ttgttcagtg cttgactgag ccagcattat aaattttctc | 5640 |
| cagatgtacc gaatcacatt tcatagcaac atgcagacat caagttttcc ctgaagctct | 5700 |
| aaccagctgg ttgcatgctg tccggagtct cagctataac ccagaagtga cctgggtcgg | 5760 |
| ggaagaggtg gtactttgcc ttctttgcac tctctgtgtt gcctcaccca ttcagcttca | 5820 |
| agcaatgtga ctgcctgacc ctgagggcgt ttacaacgcc tgacccacag accacaagtc | 5880 |
| aaccagctgg tgtgctcacg atacctagtc tgaaccatag ccctgctccc accctgcctc | 5940 |
| catctccacc ctttcttcac tgctcatcac agctggctag caaagactgc ctcagacctg | 6000 |
| agcacaggct ccactccaca gccgtgactg ttcgagccac ttaaatcaaa gagcgcttgt | 6060 |
| cttccgctca gtaaatctct cctcagctca ctgatgacgt tgactttctc tagacagcac | 6120 |
| atttgggttt aagacactgc tacttgagct cttcattcag ttcctcagaa tacctcattt | 6180 |
| gggtcagatt cccaaagagg aagatagggt tcctggcaga cagacatgtc tcattccttt | 6240 |
| gaaatccttc agagaaatgc agtgactatg gcaccttctt aaaaagcaca cacacaaata | 6300 |
| acacacacac acacacacac acacacacac acacacacac atatccccct cactgtcatc | 6360 |
| cttgatatgt atatgatata tataaaatca ttgttttata ctgtgataat tgattatgaa | 6420 |
| taaaatttac taaaatgaac aattaaaatt atgggggggg ctggagagat ggctcatcag | 6480 |
| ttaagagaac agttgctgct cttgcagaac acgagagttc agttcccagc acccacatca | 6540 |
| ggcagctcat aaccatgtgt ggtgtcagtt ccaggagatc tggtgccctc ttctggcctc | 6600 |
| ctccagcacc tgctacatgt ggttcacaca cacacacaca cacacacaca cacacacaca | 6660 |
| cacacacaca caaataaata taaagattat ttttttcaaa actgagttaa aaataggttc | 6720 |
| tatctgattc atactaaggc ttttcacagt ggttaagtct attagatatg tctagccata | 6780 |
| tcctttctcc cttctttctt gaggagaggc ttttaaagct acaagttaca gccttctttg | 6840 |
| caaataagag taccatttaa caggcctctg accaatgaga tcccagaatc ggttgcccag | 6900 |
| gagcttccca aacagtccat tatagggaaa ggtggtacaa accagtagat taggcatgtt | 6960 |
| ccacttccta agtgccgtgc caaataagga aatggcctca aatgtttgcc ttttatcttc | 7020 |
| acccacctct gaattgcacg ctagt | 7045 |
| <211> 13515 | |
| <212> DNA | |
| <213> Cricetulus griseus | |
| <400> 5 | |
| (SEQ ID NO: 9) | |
| tctagaaaca aaaccaaaaa tattaagtca ggcttggctt caggtgctgg ggtggagtgc | 60 |
| tgacaaaaat acacaaattc ctggctttct aaggcttttt cggggattca ggtattgggt | 120 |
| gatggtagaa taaaaatctg aaacataggt gatgtatctg ccatactgca tgggtgtgta | 180 |
| tgtgtgtgta tgtgtgtctg tgtgtgtgcc cagacagaaa taccatgaag gaaaaaaaca | 240 |
| cttcaaagac aggagagaag agtgacctgg gaaggactcc ccaatgagat gagaactgag | 300 |
| cacatgccag aggaggtgag gactgaacca ttcaacacaa gtggtgaata gtcctgcaga | 360 |
| cacagagagg gccagaagca ctcagaactc cagggggtca ggagtggttc tctggaggct | 420 |
| tctgcccttg gaggttcctg aggaggaggc ttccatattg aaaatgtagt tagtggccgt | 480 |
| ttccattagt acagtgacta gagagagctg agggaccact ggactgaggc ctagatgctc | 540 |
| agtcagatgg ccatgaaagc ctagacaagc acttccgggt ggaaaggaaa cagcaggtgt | 600 |
| gaggggtcag gggcaagtta gtgggagagg tcttccagat gaagtagcag gaacggagac | 660 |
| gcactggatg gccccacttg tcaaccagca aaagcttgga tcttgttcta agaggccagg | 720 |
| gacatgacaa gggtgatctc ggtttttaaa aggctttgtg ttacctaatc acttctatta | 780 |
| gtcagatact ttgtaacaca aatgagtact tggcctgtat tttagaaact tctgggatcc | 840 |
| tgaaaaaaca caatgacatt ctggctgcaa cacctggaga ctcccagcca ggccctggac | 900 |
| ccgggtccat tcatgcaaat actcagggac agattcttca ctaggtactg atgagctgtc | 960 |
| ttggatgcaa atgtggcctc ttcattttac tacaagtcac catgagtcag gaggtgctgt | 1020 |
| ttgcacagtg tgactaagtg atggagtgtt gactgcagcc attcccggcc ccagcttgtg | 1080 |
| agagagatcc ttttaaattg aaagtaagct caaagttacc acgaagccac acatgtataa | 1140 |
| actgtgtgaa taatctgtgc acatacacaa accatgtgaa taatctgtgt acatgtataa | 1200 |
| actgtgtgaa taatctgtgt gcagcctttc cttacctact accttccagt gatcaggttt | 1260 |
| ggactgcctg tgtgctactg gaccctgaat gtccccaccg ctgtcccctg tcttttacga | 1320 |
| ttctgacatt tttaataaat tcagcggctt cccctctgct ctgtgcctag ctataccttg | 1380 |
| gtactctgca ttttggtttc tgtgacattt ctctgtgact ctgctacatt ctcagatgac | 1440 |
| atgtgacaca gaaggtgttc cctctggaga catgtgatgt ccctgtcatt agtggaatca | 1500 |
| gatgccccca aactgttgtc cagtgtttgg gaaagtgaca cgtgaaggag gatcaggaaa | 1560 |
| agaggggtgg aaatcaagat gtgtctgagt atctcatgtc cctgagtggt ccaggctgct | 1620 |
| gacttcactc ccccaagtga gggaggccat ggtgagtaca cacacctcac acatactata | 1680 |
| tccaacacac acacacacac acacacacac acgcacgcac gcacgcacgc acgcacacat | 1740 |
| gcacacacac gaactacatt tcacaaacca catacgcata ttacacccca aacgtatcac | 1800 |
| ctatacatac cacacataca cacccctcca cacatcacac acataccaca cccacacaca | 1860 |
| gcacacacat acataggcac acattcacac accacacata tacatttgtg tatgcataca | 1920 |
| tgcatacaca cacaggcaca cagacaccac acacatgcat tgtgtacgca cacatgcata | 1980 |
| cacacacata ggcacacatt gagcacacac atacatttgt gtacgcacac tacatagaca | 2040 |
| tatatgcatt tgtatatgca cacatgcatg cacacataca taggcacaca tagagcacac | 2100 |
| acatacattt gtgtatgcac acatgcacac accaatcaca tcggaagact aagattgctg | 2160 |
| actaaggttc acatgaactt agcagttcct ggttatctcg tgaaacttgg caggttcttc | 2220 |
| tggagaagag gaagcgttgg cttgagccct ggcagcaatt aaccccgccc agaagaagta | 2280 |
| ggtttaaaaa tgagagggtc tcaatgtgga acccgcaggg cgccagttca gagaagagac | 2340 |
| ctacccaagc caactgagag caaaggcaga gggatgaacc tgggatgtag tttgaacctc | 2400 |
| tgtaccagct gggcttcatg ctattttgtt atatctttat taaatattct tttagtttta | 2460 |
| tgtgcgtgaa taccttgctt gcataaatgt atgggcactg tatgtgttct tggtgccggt | 2520 |
| ggaggccagg agagggcatg gatcctccgg agctggcgtt tgagacagtt gtgacccaca | 2580 |
| gtgtggggtc tgggaactgg gtcttagtgt tccgcaagtg cagctggggc tcttaacctc | 2640 |
| tgagccatcc ctccagcttc aagaaactta ttttcttagg acatggggga agggatccag | 2700 |
| ggctttaggc ttgtttgttc agcaaatact cttttcgtgt attttgaatt ttattttatt | 2760 |
| ttactttttt gggatagaat cacattctgc agctcaggct gggcctgaac tcatcaaaat | 2820 |
| cctcctgtct cagtctacca ggtgataaga ttactgatgt gagcctggct ttgacaagca | 2880 |
| ctttagagtc cccagccctt ctggacactt gttccaagta taatatatat atatatatat | 2940 |
| atatatatat atatatatat atatattgtg tgtgtgtgtt tgtgtgtgta tgagacactt | 3000 |
| gctctaaggg tatcatatat atccttgatt tgcttttaat ttatttttta attaaaaatg | 3060 |
| attagctaca tgtcacctgt atgcgtctgt atcatctata tatccttcct tccttctctc | 3120 |
| tctttctctc ttcttcttct cacccccaag catctatttt caaatccttg tgccgaggag | 3180 |
| atgccaagag tctcgttggg ggagatggtg agggggcgat acaggggaag agcaggagga | 3240 |
| aagggggaca gactggtgtg ggtctttgga gagctcagga gaatagcagc gatcttccct | 3300 |
| gtccctggtg tcacctctta cagccaacac cattttgtgg cctggcagaa gagttgtcaa | 3360 |
| gctggtcgca ggtctgccac acaaccccaa tctggcccca agaaaaggca cctgtgtgtg | 3420 |
| actctggggt taaaggcgct gcctggtcgt ctccagctgg acttgaaact cccgtttaat | 3480 |
| aaagagttct gcaaaataat acccgcagag tcacagtgcc aggttcccgt gctttcctga | 3540 |
| agcgccaggc acgggttccc taggaaatgg ggccttgctt gccaagctcc cacggcttgc | 3600 |
| cctgcaaacg gcctgaatga tctggcactc tgcgttgcca ctgggatgaa atggaaaaaa | 3660 |
| gaaaaagaag aagtgtctct ggaagcgggc gcgctcacac aaacccgcaa cgattgtgta | 3720 |
| aacactctcc attgagaatc tggagtgcgg ttgccctcta ctggggagct gaagacagct | 3780 |
| agtgggggcg gggggaggac cgtgctagca tccttccacg gtgctcgctg gctgtggtgc | 3840 |
| atgccgggaa ccgaaacgcg gaactaaagt caagtcttgc tttggtggaa ctgacaatca | 3900 |
| acgaaatcac ttcgattgtt ttcctctttt tactggaatt cttggatttg atagatgggg | 3960 |
| gaggatcaga gggggagggg aggggcgggg agacggaggg aggaggggag gaggggagga | 4020 |
| ggggaggagg ggaggagggg aagggatgga ggaaaatact aacttttcta attcaacatg | 4080 |
| acaaagattc ggagaaagtg caccgctagt gaccgggagg aggaatgccc tattgggcat | 4140 |
| tatattccct gtcgtctaat ggaatcaaac tcttggttcc agcaccaagg attctgagcc | 4200 |
| tatcctattc aagacagtaa ctacagccca cacggaagag gctatacaac tgaagaaata | 4260 |
| aaattttcac tttatttcat ttctgtgact gcatgttcac atgtagagag ccacctgtgt | 4320 |
| ctaggggctg atgtgctggg cagtagagtt ctgagcccgt taactggaac aacccagaac | 4380 |
| tcccaccaca gttagagctt gctgagagag ggaggccctt ggtgagattt ctttgtgtat | 4440 |
| ttatttagag acagggtctc atactgtagt ccaagctagc ctccagctca cagaaattct | 4500 |
| cctgttccgg tttccaaagt actggagtta tgagtgtgtg ttaattgaac gctaagaatt | 4560 |
| tgctgattga agaaaacctc aagtgggttt ggctaatccc cacgacccca gaggctgagg | 4620 |
| caggaggaat gagagaattc aaggtttgcc agagccacag ggtgagctca atgtggagac | 4680 |
| tgtgagggtg agctcaatgt ggagactgtg agggtgagct caatgtggag actgtgaggg | 4740 |
| tgagctcaat gtggagactg tgagggtgag ctcaatgtgg agactgtgag ggtgagctca | 4800 |
| atgtggagac ctgtatcaag ataataatag tagtagtaac aatgcaggcg agggtgtggt | 4860 |
| tgagtggtag agcagttagt tgatttgaca tgcttgaggt ctcccggtcc atctgtggcc | 4920 |
| ctgcaacagg aagggaggga ggaagggggg gaacgagaga gaggaaagag agacagaagc | 4980 |
| taagataggg aatgagagag gaaggaagaa acgggaagaa attcagactc cttcctgagt | 5040 |
| tccgccaacg cctagtgaca tcctgtgcac accctaaggt ggcctttgtg tggcactggc | 5100 |
| ttgggtggtc gggaaaggca ttttcagctt gttgcagaac tgccacagta gcatgctggg | 5160 |
| tccgtgaaag tttctgcccg ttaacaagaa gtctctacta cttgtgacct caccagtgaa | 5220 |
| aatttcttta attgtctcct ggtgttctgg gttttgcatt tttgtttcta aggatacatt | 5280 |
| cctgggtgat gtcatgaagt ccccaaagac acagtggggc tgtgttggat tgggaaagat | 5340 |
| gatttatctg gggtgtcaaa aggaaaagaa gggaaacagg cacttgggaa aatgtcctcc | 5400 |
| cgcccacccg aattttggct tggcaaccgt ggtggaggag caagaaacac gtggacgttt | 5460 |
| gaggaggcat ggggtcctag gaggacagga agcagaagga gagagctggg ctgacagcct | 5520 |
| gcaggcattg cacagtttca gaaggagatt acagcatgac tgagttttta gggatccaac | 5580 |
| agggacctgg gtagagattc tgtgggctct gaggcaactt gacctcagcc agatggtatt | 5640 |
| tgaataacct gctcttagag ggaaaacaga catagcaaac agagccacgt ttagtgatga | 5700 |
| aactctcact ttgcctgagt catgtgcggc catgcccagg ggtcaggctg acactcaact | 5760 |
| caaaaacaag tgagaaattg aagacaatcc gtggtggcag ctactggaag ggccaccaca | 5820 |
| tccccagaaa gagtggagct gctaaaaagc catttgtgat aggcacagtt atcttgaatg | 5880 |
| catggagcag agattacgga aaaatcgaga atgttaatga ggcaacattc gagttgagtc | 5940 |
| attcagtgtg ggaaacccag acgcttccat cccctaaaag gaacatcttg ctctcagtca | 6000 |
| aaatggaaat aaaaattggg gcttgaattt ggcaaatgat tcagaactct gtgtaggtat | 6060 |
| tttcacacgc acagtggata attttcatgt tggagtttat ttgtgctaaa aggcagaaaa | 6120 |
| gggtaaaaag cacatcttaa gagttatgag gttctacgaa taaaaataat gttacttaca | 6180 |
| gctattcctt aattagtacc cccttccacc tgtggtaatt tcctgagata gtcagtgggg | 6240 |
| aaaagatctc tccttctctt ctttctcccc ctcccctcct ctccctccct ccctccctcc | 6300 |
| ctccctcctc tccctccctc cccctttcct tctttctttg ctccttctcc tctgcctcct | 6360 |
| tctccctttc ttcttcattt attctaagta gcttttaaca gcacaccaat tacctgtgta | 6420 |
| taacgggaaa acacaggctc aagcagctta gagaagattg atctgtgttc actagcgtgc | 6480 |
| aattcagagg tgggtgaaga taaaaggcaa acatttgagg ccatttcctt atttggcacg | 6540 |
| gcacttagga agtggaacat gcctaatcta ctggtttgta ccacctttcc ctataatgga | 6600 |
| ctgtttggga agctcctggg caaccgattc tggcatctca ttggtcagag gcctgttaaa | 6660 |
| tggtactctt atttgcaaag aaggctgtaa cttgtagctt taaaagcctc tcctcaagaa | 6720 |
| agaagggaga aaggatatgg ctagacatat ctaatagact taaccactgt gaaaagcctt | 6780 |
| agtatgaatc agatagaacc tatttttaac tcagttttga aaaaaataat ctttatattt | 6840 |
| atttgtgtgt gtgtgtgtgt gtgtgtgtgt gtgtgtgtgt gtgtgtgtgt gaaccacatg | 6900 |
| tagcaggtgc tggaggaggc cagaagaggg caccagatct cctggaactg acaccacaca | 6960 |
| tggttatgag ctgcctgatg tgggtgctgg gaactgaact ctcgtgttct gcaagagcag | 7020 |
| caactgttct cttaactgat gagccatctc tccagccccc cccataattt taattgttca | 7080 |
| ttttagtaaa ttttattcat aatcaattat cacagtataa aacaatgatt ttatatatat | 7140 |
| catatacata tcaaggatga cagtgagggg gatatgtgtg tgtgtgtgtg tgtgtgtgtg | 7200 |
| tgtgtgtgtg tgtgttattt gtgtgtgtgc tttttaagaa ggtgccatag tcactgcatt | 7260 |
| tctctgaagg atttcaaagg aatgagacat gtctgtctgc caggaaccct atcttcctct | 7320 |
| ttgggaatct gacccaaatg aggtattctg aggaactgaa tgaagagctc aagtagcagt | 7380 |
| gtcttaaacc caaatgtgct gtctagagaa agtcaacgtc atcagtgagc tgaggagaga | 7440 |
| tttactgagc ggaagacaag cgctctttga tttaagtggc tcgaacagtc acggctgtgg | 7500 |
| agtggagcct gtgctcaggt ctgaggcagt ctttgctagc cagctgtgat gagcagtgaa | 7560 |
| gaaagggtgg agatggaggc agggtgggag cagggctatg gttcagacta ggtatcgtga | 7620 |
| gcacaccagc tggttgactt gtggtctgtg ggtcaggcgt tgtaaacgcc ctcagggtca | 7680 |
| ggcagtcaca ttgcttgaag ctgaatgggt gaggcaacac agagagtgca aagaaggcaa | 7740 |
| agtaccacct cttccccgac ccaggtcact tctgggttat agctgagact ccggacagca | 7800 |
| tgcaaccagc tggttagagc ttcagggaaa acttgatgtc tgcatgttgc tatgaaatgt | 7860 |
| gattcggtac atctggagaa aatttataat gctggctcag tcaagcactg aacaaaggta | 7920 |
| ccttggcttt gggagctaca tgacattgac ttgtaggcag actttttttt ttctgcccgc | 7980 |
| caattcccag ataaccaata tggaggctca atattaatta taaatgctcg gctgatagct | 8040 |
| caggcttgtt actagctaac tcttccaact taaatgaacc catttctatt atctacattc | 8100 |
| tgccacgtga ctttaccttg tacttcctgt ttcctctcct tgtctgactc tgcccttctg | 8160 |
| cttcccagag tccttagtct ggttctcctg cctaacctta tcctgcccag ctgctgacca | 8220 |
| agcatttata attaatatta agtctcccag tgagactctc atccagggag gacttgggtg | 8280 |
| ctcccccctc ctcattgcca tccgtgtctt cctcttccct cgcttccccc tcctcttcct | 8340 |
| gctcttcctc ctccacccct cctttcatag tattgatggc aagggtgttc tagaatggag | 8400 |
| gagtgcccat aggcatgcaa agaaaccagt taggatgctc tgtgaggggt tgtaatcata | 8460 |
| agcgatggac acaattcaag ccacagagtg aagacggaag gatgcactgt gctctagagc | 8520 |
| aacttctggg gcagaatcac agggtgagtt tctgacttga gggcgaagag gccacgagga | 8580 |
| agggagtgag tttgtctgag ctagaagcta cggcccacct cttggtagca gacctgccca | 8640 |
| caagcatgct ttgttaatca tgtgggatct gattttcctc taaatctatg ttcaactctt | 8700 |
| aagaaaatgt gaattctcac attaaaattt agatatacgt cttttggtgg ggggggtgta | 8760 |
| aaaaatcctc aagaatatgg atttctgggg gccggagaga tggctcagag gttaagagaa | 8820 |
| ctggttgctc ttctagacat tctgagttca attcccagca accacatggt ggctcacaac | 8880 |
| catctgtaat gcgacctggt gccatcttct gacatgcatg gatacatgca ggcagaaagc | 8940 |
| tgtatacata gtaaattgat aaatcttttt ttaaaaagag tatggattct gccgggtgtt | 9000 |
| ggtggcgcac gcctttaatc ccagcactct ggaggcagag gcaggtggat ctctgtgagt | 9060 |
| tcgagaccag cctggtctat aagagctagt tccaggacag cctccaaagc cacagagaaa | 9120 |
| ccctgtctcg aaaaaccaaa aaaaaaaaaa aaaaaaaaaa aaaaaaaaga gtatggattc | 9180 |
| taagaaagcc gtaacagctg gagctgtgta cggagttcag cgtggtacta gaagaacaga | 9240 |
| cattcatgat gaaacacccc aggattttta cttagtatct agtttccatt gttgttttga | 9300 |
| gaccggctct tatgctctcc aggctggcct caaactgctg atcttcccgc ctctacctct | 9360 |
| caagtcctgg gactacttgg ctcataaaac agtttttgtc gggctccctg aagttatggt | 9420 |
| tgtacaaacc gtgggggtca atatactcac ttgggcagag agagaaggtc tgaatcccag | 9480 |
| acaatgactg catctcagga cagttgggaa gaggacaatg gcagaaggac ttagaaaaga | 9540 |
| tagactggag ggtggaaaag cagcaggaac agagaaacaa aacaggaagc ttgctatcca | 9600 |
| gggccactct ggagtcctgt ggcaagatgg aagcgggcta ggggaataca tttgtgctac | 9660 |
| tgtgtgtgtg tgtgtgtgtg tgtgtgtgtg tgtgtgtgat caatgcctat caatgttgaa | 9720 |
| ggggaaatat gtataccaca ttgattctgg gagcaattct cagtatctgg cctagagaaa | 9780 |
| ggaatggccc ctgcagaata gacagagtga atggtgccct ttatcatttg ctaaagtgaa | 9840 |
| ggagaaataa acatccttcc atagagtttc aggtaaatga accccacagt tcatctgtgc | 9900 |
| cgtggtggag gcctggccaa cagttaaaaa gattagacac ggacaaagtc tgaaggaaac | 9960 |
| acctcgaata ggaagaggag agccacctca ttctgtaact ttcctcaagg ggaagatgtt | 10020 |
| ccaagagtgg gaataaatgg tcaaaggggg gatttttaat taggaaaacg atttcctgta | 10080 |
| tcacttgtga aactggaggt tgatttgggg cataggacaa tagatttgat gctttgcaaa | 10140 |
| aagctgtttc aaagcagaga aatggaatag agacaattat gtagcgagga gggagggtgg | 10200 |
| ggcgaagaty gagacagaga agtggaagct gactttaggg aagaggaaca tagaccacag | 10260 |
| gggcggggcg gggggcaggg gcggggggcg gggctcaaag gaggcagtgg gaacgttgct | 10320 |
| agtgttcgca gcgtaagcgt gaatgtgcaa gcgtctttgt ggtgtgtgac caggagtagc | 10380 |
| gtggctggct tgtgtgctgc ttgtaatccc agtctttgag gtttccacac tgttccacag | 10440 |
| tgggtgtgat tttccctcgg agagcatgag ggctctgctt tccccacatc ctccccagcg | 10500 |
| ttcgttggta tttgtttcca agatgttagt gggtgagaca aagcctctct gttgatttgc | 10560 |
| ctttaacagg tgacaaaaaa agctcaacca ggagacattt ttgccttctt ggaaggtaat | 10620 |
| gctcccatgt agagcaatgg gacccatctc taaggtgagg ctactcttgc agtttgcacc | 10680 |
| cagctcttct gatgcaggaa ggaagttggt gggcaagcaa gactgtttgc ttcttgcgat | 10740 |
| ggacacattc tgcacacaaa ggctcaggag gggagaaggc tgtttgatgt ttagcactca | 10800 |
| ggaaggcccc tgatgcatct gtgattagct gtctccatct gtggagcaga cacggactaa | 10860 |
| ctaaaaacca gtgtttttaa attgtcaagc ctttaaggtg aggaaattga cttattgtgc | 10920 |
| tgggccatac gtagagcaag tgctctgcat tgggccaacc cccggctctg gtttctaggc | 10980 |
| accagaatgg cctagaacta actcacaatc ctcccattcc aggtctcagg tgctagaatg | 11040 |
| aaccactata ccagcctgcc tgcctgccta cctgccttcc taaattttaa atcatgggga | 11100 |
| gtaggggaga atacacttat cttagttagg gtttctattg ctgtgaagag acaccatgag | 11160 |
| catggcaact cttataaagg aaaacattta gttgggtggc agtttcagag gttttagtac | 11220 |
| attgtcatca tggctgggaa catgatggca tgcagacaga catggtgctg gagaaaggga | 11280 |
| tgagagtcct acatcttgca ggcaacagga cctcagctga gacactggct ggtaccctga | 11340 |
| gcataggaaa cctcacagcc caccctcaca gtgacatatt tccttcaaca aagccatacc | 11400 |
| tcctaatagt gccactccct atgagatgac agggccaatt acattcaaac tgctataaca | 11460 |
| ctttaaagta ttttattttt attattgtaa attatgtatg tagctgggtg gtggcagccg | 11520 |
| aggtgcacgc ctttaatccc agcacttggg aggcagaggc agatggatct ctgtgagttc | 11580 |
| aagaccagcc tggtctataa gagctagttg caaggaagga tatacaaaga acagttctag | 11640 |
| gatagccttc aaagccacag agaagtgctg tcttgaaaac caaaaattgt gctgggacct | 11700 |
| gtctctgctt tggttgcttc ccactccccc agagctggac tcttggtcaa cactgaatca | 11760 |
| gctgcaaaat aaactcctgg attcctctct tgtaacagga gcccgaagtc aggcgcccac | 11820 |
| ttgtcttctc gcaggattgc catagacttt ttctgtgtgc ccaccattcc agactgaagt | 11880 |
| agagatggca gtggcagaga ctgggaaggc tgcaacgaaa acaggaagtt attgcaccct | 11940 |
| gggaatagtc tggaaatgaa gcttcaaaac ttgcttcatg ttcagttgta cacagactca | 12000 |
| ctcccaggtt gactcacacg tgtaaatatt cctgactatg tctgcactgc ttttatctga | 12060 |
| tgcttccttc ccaaaatgcc aagtgtacaa ggtgagggaa tcacccttgg attcagagcc | 12120 |
| cagggtcgtc ctccttaacc tggacttgtc tttctccggc agcctctgac acccctcccc | 12180 |
| ccattttctc tatcagaagg tctgagcaga gttggggcac gctcatgtcc tgatacactc | 12240 |
| cttgtcttcc tgaagatcta acttctgacc cagaaagatg gctaaggtgg tgaagtgttt | 12300 |
| gacatgaaga cttggtctta agaactggag caggggaaaa aagtcggatg tggcagcatg | 12360 |
| tacccgaaat cccagaactg gggaggtaga gacggatgag tgcccggggc tagctggctg | 12420 |
| ctcagccagc ctagctgaat tgccaaattc caactcctat tgaaaaacct ttaccaaaca | 12480 |
| aacaaacaaa caaataataa caacaacaac aacaacaaac taccccatac aaggtgggcg | 12540 |
| gctcttggct cttgaggaat gactcaccca aacccaaagc ttgccacagc tgttctctgg | 12600 |
| cctaaatggg gtgggggtgg ggcagagaca gagacagaga gagacatgac ttcctgggct | 12660 |
| gggctgtgtg ctctaggcca ccaggaactt tcctgtcttg ctctctgtct ggcacagcca | 12720 |
| gagcaccagc acccagcagg tgcacacacc tccctccgtg cttcttgagc aaacacaggt | 12780 |
| gccttggtct gtctattgaa ccggagtaag ttcttgcaga tgtatgcatg gaaacaacat | 12840 |
| tgtcctggtt ttatttctac tcttgtgata aaaaccgggg aactccagga agcagctgag | 12900 |
| gcagaggcaa atgcaaggaa tgctgcctcc tagcttgctc cccatggctt gccgggcctg | 12960 |
| ctttctgcaa gcccttctct ccccattggc atgcctgaca tgaacagcgt ttgaaatgct | 13020 |
| ctcaaatgtc actttcaaag aaggcttctc tgatcttgct aactaaatca gaccatgttt | 13080 |
| caccgtgcat tatctttctg ctgtctgtct gtctgtctgt ctgtctatct gtctatcatc | 13140 |
| tatcaatcat ctatctatct atcttctatt tatctaccta tcattcaatc atctatcttc | 13200 |
| taactagtta tcatttattt atttgtttac ttactttttt tatttgagac agtatttctc | 13260 |
| tgagtgacag ccttggctgt cctggaaccc attctgtaac caggctgtcc tcaaactcac | 13320 |
| agagatccaa ctgcctctgc ctctctggtg ctggggttaa agacgtgcac caccaacgcc | 13380 |
| ccgctctatc atctatttat gtacttatta ttcagtcatt atctatcctc taactatcca | 13440 |
| tcatctgtct atccatcatc tatctatcta tctatctatc tatctatcta tctatcatcc | 13500 |
| atctataatc aattg | 13515 |
| <211> 14553 | |
| <212> DNA | |
| <213> Mus musculus | |
| <400> 6 | |
| (SEQ ID NO: 10) | |
| cttgaagaac acatgttttc caagagggag cacccatgtt ggaatgacaa tgtagttagt | 60 |
| gctcctctcc tgtaggttag tgctcctttg ctataggtaa gtgctcctct cctataggtc | 120 |
| agtgctcctc tcctataggt tagtgctcct ctcctatagg ttagtgctcc tctcctacag | 180 |
| gttagtgctc ctctgctcta ggttagtcct gctctcctat agtacctaga gagctagggc | 240 |
| aaatgggcta ggcccgaagt gcagagacaa acagctatgg aagactgggt aagcacttcc | 300 |
| aagctacgaa agagcagtgt gaagggtcag ggcttgtgca gttagtaggg gagatcttcc | 360 |
| agttgaagaa acagaagaac tgagagccac tgggtatcat cctcctgcgc catgccttcc | 420 |
| tggatactgc catgctccca ccttgatgat aatggaatga acctctgaac ctgtaagcca | 480 |
| gccccaatga aatattgttt ttatgagagt tgccttggtc atgctgtctg ttcacagcag | 540 |
| taaaacccta aataaggcag aagttggtac cagtattgct gtgatagacc tgaccatgct | 600 |
| ttcctttgaa agaatgtgga tttggtgact ttggatttgc aacacagtgg aatgctttaa | 660 |
| atggagatta atgggtcatc aattcctagt aggaatatgg aagactttgt tgctgggagt | 720 |
| atttgaactg tgttgacctg gcctaagaga tttcaaagga gaagaatttc agaatgtggc | 780 |
| ataaagacag tttttgtggt attttggtga agaatgtggc tactttttgc ccttgtctga | 840 |
| aaagtctgcc tgagactaaa gtgaagagaa tcagattaat tgcattgaca agggaagttt | 900 |
| gtggctgcgc tatctggaaa cttacagcca gcctcttgga cctcgggtga cttacgcaaa | 960 |
| tactcaggga cagagatgct tgactctgta ctgatgagtt gtcttggatg caaatatggg | 1020 |
| ctcttcattt gactacatgt cacgatgagt caggagctgc tctctccaga gtgtgacaaa | 1080 |
| gcgaggggat gctgacggta gctgttctag ctttgaaggt aagcctgcac ttatgctaaa | 1140 |
| gtcacacata cacgagccgg gtggagaacc tgtctgtgtg gagacacctt tcattacctg | 1200 |
| tggcatccag cctctcaagc ttggactgcc tgtgtgctcc tggactctgg aggtcccact | 1260 |
| gctctgtcct ctgctgctta tgatactgac attttaaaag aatccagtgg ttcccccctg | 1320 |
| tactcggtgt ctacttctac ctggatgttc ctcatttatg ttctgtgaca cttctctgtg | 1380 |
| actctgctgc attcctgggt gacatgtgga caccctgtcc ctttgcagac catgatgtca | 1440 |
| ctgtcactag tggaatcaga tgccccaagt gttgtcctgt gtttgggaac gtgacaggca | 1500 |
| gtacagaagc agaagaggaa gggtgaaaac ggaaatgtca cagcagcatc tgatgtgtgc | 1560 |
| ctcagtcacg catgctgctg attggaacta ctcagcatga gagagggcca tggtgaatac | 1620 |
| acaaccctat acacactgtg tccatttctc tctctctctt acacagagag agagggagga | 1680 |
| gggggagggg gaggcggagg gggaggggga gggagaggga gtgggagagg gagagggaga | 1740 |
| gggagaggga gagggagagg gagagggaga gggagagttt aatgtctgtg aagagatacc | 1800 |
| atgaccaaag caactcttat aaaggacaac atttaattgg ggctggctta caggttcaga | 1860 |
| aattcagtcc attctcacca tggtgggaag catgcaggta gatgtggtgc tggaggaacc | 1920 |
| aagagttcta tatcctgatc tgaaggcagc caggagaaga ctgcctcttc tgcacagggc | 1980 |
| agagcttgag catagaacat caaagccctt ccccacactt cctccaacaa ggtcatacat | 2040 |
| acttcaacaa agacacacct cctaacggtg ccactccctg tggaccaacc atttaaacgc | 2100 |
| atgagtctat gagggtcaaa gctcttcaaa ccaccacact catgtacaca cacacacaca | 2160 |
| cacacacaca ctctcataca cacacacaca cacactcaca cacacacaca cacacacaca | 2220 |
| cacacacaca ccacacacac acacacacac agagttctat tttgcactgt ttcactgtca | 2280 |
| caaggttcta cttatctcag acacactgcc aggaattgtg tcggaagact ttcagtttct | 2340 |
| ttgggttcac atggacttag cagttcttgg tgatcctgaa agatttctgc agaaagaagc | 2400 |
| caaagtgttg agcccaaggc ctggccacac attagtcctg tctagatgaa caggggttta | 2460 |
| aaaataaggg tcgaagtcaa tgaagccagc aggggctgac ttagagagga gacccaccca | 2520 |
| agccaactgc ggcatcaagg aagcgatgaa tccccatatc cagctgtgcc cggtgctgtc | 2580 |
| ttgctacatc tttagtaaat gttcttttag ttgtatgcgt atgaatattt tgcttgcata | 2640 |
| tatttgtgta caccataggt gttcctaggg cctatggagg ccagaagagg gcatcagatc | 2700 |
| ctttggaact ggaattatag acacttgtta cccatagagt agattgtggg aaatgagcct | 2760 |
| ttagtcttcg agagcggcca gtgctcttaa cctttggtcg tttctccagg tctttgagac | 2820 |
| tttattttct tggacatcag gacaggatcc agggctttga gcttgtttct tcagccagct | 2880 |
| ttcttttcat gtatattaaa ttttatgtta ttttgctttc tttttcccca agacagaatc | 2940 |
| acactctata tagctcaggc tgggtttgaa ttcagtttcc ctgtctcagt ctaccgggta | 3000 |
| atatgattac agatgtgagt ctgactttgg tatcaaagtc cccagccctt ctggatatgt | 3060 |
| gttttaagga tatcagatat atccttgatt tgctttgaat tttcttttta gttacaacat | 3120 |
| aattagttcc gtgtcacctg aatatgtgta tgtcacctac atagtcttcc ttcttctctt | 3180 |
| cttccctctc ccaccttccc aggtacctgt ctgtcttcat atccttgtgc tgagagtctt | 3240 |
| gttgagggag atgatgaccg agacagagcc actggggaag ggagatgggc tagtgcaggt | 3300 |
| cttcagagag gagctcgtga atattgtagc ccctttagtc cctggcatgt cctcttgtat | 3360 |
| agccaccgcc atgctgtggc ctggcagaag tgaataagtt gtccagctgt tgacaggcct | 3420 |
| gccctccaga cccagtctga tcccaagaaa gggcatctgt gtctgtctct gaggccgtaa | 3480 |
| gtgctgcctg gttgtctcca gcttgacttg acactccctc cttaataaga gtaccacaga | 3540 |
| acagggtctg cagagtccct gggccaggtc cctgtgctgt cctggaatgc caggcgtgaa | 3600 |
| tttcctgtga agtaggactt tgctcgccaa gctcccacgg cttgcccttc agatagccag | 3660 |
| aattatctgg taccctgcat tgccgttcaa tacgcagagt atcactggaa gcgcgcgcgc | 3720 |
| gcacacacac acacacacac acacacacac acacacacac acacgcccac tccatcttta | 3780 |
| aaccccaccc cccagcaacg gcggtgtaaa cactctccat caggaagctg aaacgcagtt | 3840 |
| gccctctgct ggggagatga aggcagcttg ctgggggcga ggaccgtgct agcaaccttc | 3900 |
| cctggtgcac acgggctctg gtgcatgacg ggaacggaaa cgcggaacta aagtcagtcc | 3960 |
| tgcttttttt tttttttttt tttttttttt tttttttttt tttttttttt ggcgttggtg | 4020 |
| gtggactgag tgacaatcag tgaaatcact taggttgttt ttctcttctt cgttgggttt | 4080 |
| gatagacggt gggagagggt cagaggagaa ggggagggat ggggagagag ggaggaggga | 4140 |
| ggggcgggag gcggggggcg aggaaaacgt gctaacttct ccaatcctac aagacaaagg | 4200 |
| tttggagaaa gccgcactga gtgacccagc agaaggaatc caggaatgtc cgctggaatc | 4260 |
| tgactgttga ttccagcgcc atgcagagaa tctaggctgg taggaacatt ctttgtccta | 4320 |
| tccgacataa taactccaac caacacggaa aagaaaggct atacaagtga agaaatggca | 4380 |
| ttttcacttt catgactata caatcacttc caggtagtaa cacgtgtcta gcacagcggt | 4440 |
| tctcaacctg ggggtcacga tcccccactt ttctgcatat cagacatttt tacgttgtta | 4500 |
| ttcataacag tagcaaaatt gcagctatga agtaacaatg aaatgcattt atggtgcgtg | 4560 |
| tgtgtgtgtg tgggggggta tcaccttaac atttactgta agaaggttga gaatactgct | 4620 |
| ccagcagcta gtgtgttgga cttaggttct gggtatatta ttagcaatag ccaaccagaa | 4680 |
| tccccaccca ccacagcatt gaggccccat gcagggcttg ctgggagagg cactgataag | 4740 |
| acttctttat gtatttattt agagacgaat actcattagg taggccaagc tagcgtcaaa | 4800 |
| ctcatggcaa ttctcctcct ccagtttcct aagtactgga ctcaggagtg tcttgccatc | 4860 |
| atatacagta aggatttatt gactgaagaa aatctcaagt ggctttggtt aatccctact | 4920 |
| acgccagagg ctgaggcagg aggcgcgcaa ggtcaaggct tgcctgggct acatatagag | 4980 |
| tgagctcaat tttgacactt ggtgcggtgt tagtagtaat agtaaagatg aaggtgtggc | 5040 |
| tcaggtgggg ccggtgattg gacacacttg gggtctcctg gtccatctgc agctgtgcaa | 5100 |
| caggaagagc ggagaatgag aggaaagaga gaaaagacag aatgagagag agggaggaag | 5160 |
| agagaaaaag gaaaagagag aggaaaggaa aaaggaaaat gaggaaagcg agaaagaaga | 5220 |
| aatgagaaag aggaaaggga gaaagaaatg agagagagaa aagaaaagac agaatgcgag | 5280 |
| agagggagga agagagaaaa aggaaaagag agaggaaagg aaaaaggaaa atgaggaaag | 5340 |
| cgagaaagaa gaaatgagaa agaggaaagg gagaaagaaa tgagagagag aaaagaaaag | 5400 |
| acagaatgcg agagagggag gaagagagaa aaaggaaaag agagaggaag ggaaaaagga | 5460 |
| aaatgaggaa agcgagaaag aagaaatgag aaagaggaaa gggagaaaga aatgagagag | 5520 |
| agaaaagaaa agacagaatg cgagagaggg aggaagagag aaaaaggaaa agagagagga | 5580 |
| agggaaaaag gaaaatgagg aaagcgagaa agaagaaatg agaaagagga aagggagaaa | 5640 |
| gaaatgagag agagaaaaga aaagacagaa tgcgagagag ggaggaagag agaaaaagga | 5700 |
| aaagagagag gaagggaaaa tggaaaatga ggaaagcgag aaagaagaaa tgagaaagag | 5760 |
| gaaagggaga aagaaatgag cgagataaaa gacagaattt gagagaggga ggaagaaata | 5820 |
| ggaaaagaga ggaaaggatg gagaaaagag agaaagaaag agagatgaaa gagagaaagg | 5880 |
| agaaatgaaa tgagagagag agagagacac aaagagccag agagagaaga aaaaagggga | 5940 |
| aagagaaaga gaaagaggaa ggctcctctt ggacacatct tcctttatct ttccctgggg | 6000 |
| accgccaaag cctggtggca tactgtacat tctgtacact gttcattcaa aacaggctct | 6060 |
| gtcttaaaga tggtctgagc ggtcagaaaa gggtattgtt aacttgtttg caaaactgcc | 6120 |
| tcaggagagt gctgagtgcg tgaaagttgc tgcccgttaa ggagaagtct ctactacttg | 6180 |
| tgatctcacc atcgaaaatt tctttaattg tctcctggtg ttctgggttt tgcagttttg | 6240 |
| tttctaagga tacattcttg ggtgatgtca caaagtcccc aaagacacgg tggagctgtg | 6300 |
| ttagatgggg aaagacagtc tgctgaggat ttatctggaa ctgtcagaag gaaaagaagg | 6360 |
| taaatggggc acttgggaaa gtggcctcta gtttgacttc tggcttagca aaggttgtgg | 6420 |
| ggagataagg catacacagt agttagcagg aggcaacagg gtcctgggag gacgcgaggc | 6480 |
| agaaggagag gctgggctga cagcatgcaa tcattgcata gtctccaaag gagattgcaa | 6540 |
| catggctgag ttttcagagg tcctacagag cccgtggtag agattctgtg ggttctgaga | 6600 |
| caacttgact ttagccagat ggtatttgag taatctggga gagagaaaac agctacagca | 6660 |
| aacagggcca catttagtga cgaaactctc actttgactg ttgagtcatt tgcagtgggc | 6720 |
| cctgaggtca ggctggccct cagctcaaaa acaagcgagg aactgaagca attactcaga | 6780 |
| taatccacag ccacagccac tggaaagggc cacatcccca gagacagcac agcaggggtg | 6840 |
| ggggtggggc tatgagaaag ttagtgattg tagcagttat ctagaatgtg cggagcagag | 6900 |
| gaggttacac aaaaacctag aatgtcattc aatgtgggaa accgagaggc tcccaagccc | 6960 |
| taaaaggaac agtttgcttt cagccaaaat ggaaataaaa tttggggctt aaatctggca | 7020 |
| aatgattcag accttctgtg taggtgtctt taaatgcaca gcagattgat tttcatgttg | 7080 |
| gagtttattt gaactaaaag acagaaatgg tgaaaagcac acctgaagaa attgagatgc | 7140 |
| tatgaataaa atcatttact tacagctatc acttaattag tacctccttc caccttgctg | 7200 |
| atttattggg ctagtcaagg aagaaaagat cttccctcct ccttctctcc tcctccccct | 7260 |
| cctctcctcc tcccctcccc tccttgacct tcctctcctc cttttccctc ctccccctct | 7320 |
| tcttctcttc accccctcct cccctcccct cctctgtact cctccccttt cctcccaatc | 7380 |
| tcttttttct cccccttctt ctctttctcc cccctcctct tccctcctct tcctccctcc | 7440 |
| ctccctcctc ctcctcatcc tcctcttcct cttcatcctc ttctccttcc tccctctcct | 7500 |
| cctcctcctt ttccagccct acctaccttc cctttcttct tcatttattc aaagtagctt | 7560 |
| tgaacagcac tactcggttt agttgtgtat aaaaggaaaa tgcaggtcca agcagcttgg | 7620 |
| ggaagattgc tttttgctct ctggaggcag atgatgacag ttcaagatca ttccttttgc | 7680 |
| tccatgtcac aggaaggggg acatgccgaa tctaccagtt tgcagccacc tacacaggat | 7740 |
| ccaccttcac ttctaaggaa atgtttggga agctacctac caaccacttc tggcatctca | 7800 |
| tgggctagag gactcttaaa tggcactctt atttgtttaa taaaggaggt tgtgacgtgt | 7860 |
| agttttaaat cccttccaca caacaattgc tactctctga ccaaaaaaga agggagacag | 7920 |
| gatacggcta ggtgtctagt agactttacc actttgaaaa gccttaatat aaatcaggta | 7980 |
| gatacatctt tttaacttat tcttgtaaag acaaaaacaa aactttattt ttatttgtgt | 8040 |
| gtatgcttgt gtgtgtgtgc ctgtgtgtat accacatgtc gctggtgccg gagaacacca | 8100 |
| gaagagggga cctgatctcc tggagctaaa gctatccatg gttctgagct gcctgatgtg | 8160 |
| ggtgctggga acagaactct ggtcttctgc aagagcaaca agcctcctct taactacgaa | 8220 |
| tctcctcccc atccccccaa atacatttaa ttattcattt tagcagcttt atttcgtaac | 8280 |
| tacttatcac agcataaaac aaggatttta tatatattac atgcaatcga ggataagagt | 8340 |
| tgaggggaga tgcgtgtgct ccttctgggt gtctgtgctt ttgaagaatg taagcagtgc | 8400 |
| acaagggacc gaggcgtgcc tgtctgccag gagctgtctt cttcccttgg actctgagct | 8460 |
| gagtgcagtg ctccgaagaa gtaaaagacg acctcatgaa gcaatgtctt caacccaaac | 8520 |
| atgctgtcca gacaaagtcc agcttcatta gtgctctgag gagagactta ctgagcctca | 8580 |
| ggaaagcccc cctcagcatg gcgaaagtcc actttgattg aagtgactcg aaagccatgg | 8640 |
| cagtgcggcg gcggccgcgt ggagcttgtg ctcgagtcgg aagcggcatc tttgtcaggc | 8700 |
| ggctgtgatt agcacgggga ggcaggactg gagtgaagga agagttgggg gcggggctta | 8760 |
| gcgctctggt ctcctaagct gtagtcagcg cctcaagatt tgtaacctgc cttctgcctt | 8820 |
| cccagccagg cagtcaagtg gctccaagct gaagactgca aagtgcccct aaccttttgg | 8880 |
| ttatagcgag gctgaagaca ccgtgctctt tcatgaaagc cggatgtctg aaatccgatt | 8940 |
| tgataaatat ggataaaacg tataacgctc gatcaatcga atcgaaggag ctcacgattg | 9000 |
| gcaccacggc tttggggaca acagagtact gactcgttgg gaggacttgg atacttcccc | 9060 |
| tcctcttcca tctcttcccc tttcctcact tcctcctcct tccttctcca ttttctccct | 9120 |
| cttcactgtt tcttactatt tttacaaaag attttattta tttatttatt tatttattta | 9180 |
| tttatttatt tatttattta tttatttaat gtatgcgagt acactgtagc tgtcttcaga | 9240 |
| cacaccagaa gagggcgtca agttccatta gagatggttt cgagccacca tgtggttgct | 9300 |
| ggggcctctg gaaggaccgc cagtgctctt aacccctgag ccatttctcc agtacccttc | 9360 |
| tcaccgtttc tcttcaatct tcttcctctt ccttctccac tttccttgtc ttcttggttt | 9420 |
| cattatcttt ctccctttct tcctcttctc cccttcttcc tcctccactg tagttttcct | 9480 |
| tccctactct tttcctgcct ccctcctcct cccctctcat tccccctcct ctttcctcct | 9540 |
| tctccctcct cctccttcct tctccctctc ccctctcccc tctcccttct cccttctccc | 9600 |
| cctcctcttc ctctttctcc ttctccaccc ctcctgtcac agtatcaatg gcaagggtgt | 9660 |
| tctagaatgg aggagtgtcc cctaggcact aacgaaagcc agttaggatg ctctgagacg | 9720 |
| ggtacaattc agggagggcc gtggggatgg aagggttgtg ctgcgattca ttctggagca | 9780 |
| acccccaggc agaatcatga ggttggttcc ggattcgcag ggcacaattc agaagaggaa | 9840 |
| ggtttcagga aggacgagtt tgtctgagat aggagttaca tctgatgtct tggcagcaga | 9900 |
| gccactgtac aagcgtgctt tattaaccac gtgggattaa atcttctttt aaatttattt | 9960 |
| tcaactctta aggaaacgtg aactttcaca ttcaaattta gacttgcagc tcttatgggg | 10020 |
| aaaaaaaggg gatcttaaga atattaagca taggcggctg gagagatggc tcagcggtta | 10080 |
| agagcactct ctgctctccc agaggtcctg agttcaattc ctagcaacca cataatagtt | 10140 |
| aacaacagtc tttaatgaat tctaatgccc tcttctggtg tgtctgaaga cagttacagt | 10200 |
| gtactcatat aaataaaata aagaaattta aaaaaatgaa tattaggcat agattcctgg | 10260 |
| atcctaagaa agccatcaga gctggagcca tgtgtgggat cctgcttggt gctggagggg | 10320 |
| cagagttcat gcccccgggg tttttactta ttatcacatt ttcatcgttg ttttgaaaca | 10380 |
| gggtcttgtg tggtccaggc tggccttgaa ctcatctttc agcctctacc tcacaggttc | 10440 |
| tgggattact tggttcctaa aagtatctcc gtcaagctcc ctggtgttat ggctgtgcca | 10500 |
| accaggaggg tctatacact cgctcaggta gagggagaag atccgaatct ctgacaggga | 10560 |
| ctgctgcctc tcggggcaaa tggagtgaag gacagcggca gaaggattta ggaaagatgg | 10620 |
| acgggagagt ggaaatgctg cagaagccag aaaacaaagc aggaagcctg ctgtccagtg | 10680 |
| gggctcaaga gcggagggat gcgagggggc tgcgcaggaa catttagcgt ctgcgtctat | 10740 |
| gggggtaggg gcggggtgcc agcacctagt cacctgaagg ggaaatgctt gcccagggag | 10800 |
| caggtctcag tagctgacct agagaaagga gcggccccta cagaggagac acgggtcact | 10860 |
| gtttgttaaa gtgaaggaga aataaatatt ctttcaaaga atcttaggtg agcccagttc | 10920 |
| atctgcgctg tggaggcctg gggaacagtt aaaaagaccc tgacacacac ccaaggcaaa | 10980 |
| caagcaacac acggctcctt ccgtaagggt ccatgattct ctgaagaatc agccccggaa | 11040 |
| tcagccccgg aatcaggtag tccgtaaaca caatgagtgt tttactctgc agaagtccag | 11100 |
| cctgctggcg tctcccatta ccaaaataga gggatagtca cgtgagctca ccggctcgat | 11160 |
| ttaaggcacg tcgttttcca gggtagatga gctttggctt ctggaaccat tatggggcac | 11220 |
| gaaggatgga gccaggattt tttttttttt tttttttttc tattagcaat tgatttgctt | 11280 |
| gggcttggct ggacttgccc agttcttagg cccagtcttc ttaactgccg atctgaagtc | 11340 |
| tgtcatggag tcagcctagc cttctcactt cccttcagct cgaataggaa gaggaggtgc | 11400 |
| acaccagatg gtctgagagc agggataaat ggtgtgcctt tgtctttcag tatttcgtta | 11460 |
| ttttaagtag gaagatgctt ttctgtatta cattgcttgt gaaaccggaa gttgattcgg | 11520 |
| ggcacaggac aatggatttg gtgttttgca aggactgttt cagaagagag aggagtggaa | 11580 |
| gggtggttag agtgaggagt ggggtgggac gggatggggg aagagaagga agggccagac | 11640 |
| aggctaggta gggctgagag gaggcggtgg gaacttcttg agttagcgca gcagtaaact | 11700 |
| tggatgtgcg tgtatctttg tgatatatga cccggagccg tgtagctggc tccgatagta | 11760 |
| ctgctaatgt cagtgtcggg gggggggggt cccatactgt tccacagggg ctgcacattc | 11820 |
| ccatcgagag caggagggct cctctctcca tacatcctcg ccagcattcc ttgttgtttc | 11880 |
| tgtgatgaca gggggtggga tgaaatctct ctgttggttt gagagaccgt gaagaagctc | 11940 |
| aaccccagga cattttgcag tcttggaagg cagtgcctcc atgtggagcc gtggagccca | 12000 |
| tctctgagtc caggtcactc ttgcagttcg cactcagctc ttcagatgca ggagagacgt | 12060 |
| tggtgggaaa gcaagattgt ttgcttgttg agatagacac attctccaca caaaggctca | 12120 |
| cgtggggcaa aggctgattg acgtacagcg ttcaggaacg cctgtggtag agctatgatt | 12180 |
| agctgtctcc atctatgaag cagacaaaga gttataaaaa aaatcaatgt tttcaaattg | 12240 |
| tcaaactttt aacccgacag caagcgctct gtccctgggc taatccctag ccctggtttc | 12300 |
| ttgagatggg gtcttttgtg cactagactg gcctagaact cacgatctta gtgttccagc | 12360 |
| ctcccagctg ctgggatgag ccgctataac cagtctgcct gccttcctaa attttaagtg | 12420 |
| atgggaagtg ggggagaata cagtttaaag tatgcagatc tgagagcagg aacctggcaa | 12480 |
| agccaagggg ccggagttac aggcggctaa catgggtgct gggaactgac ccaggtcctt | 12540 |
| gagaggagca gtgtgtactc ttgaccaaac aggtccgtct ctccagtccc cgtagtatta | 12600 |
| aaaataggta ctacgggcat ggtggtgcac acctttaatc ccagcactag ggaggcagag | 12660 |
| gcaggtggat ttctgagttt gaggccagcc tggtctacaa aatgagttcc aggacagcca | 12720 |
| cggctataca gagaaaccct gtcttgaaaa caaaacaaca acaaaatagg tactacaaag | 12780 |
| cgatgtaatt gtgctcaaac atgcaaaccg aggggactgt atgcataaga aagagaaaga | 12840 |
| cggccacact ggttctatct gggtgacagg aaatcagtat ttttattttt cacattcatt | 12900 |
| tttttgttgt tgttgttgac acagtgattt ttctatcaaa aacattattt cttttatagt | 12960 |
| tcccctgagg agctgttttt aaagccgtgc tttgaaaaac cattgaagga gcagaggcag | 13020 |
| ggagactcct gtgtggcagt cggtgaagca ggccctctgc aggcaggctg gccctggact | 13080 |
| tgggagtctc tttccctccc tcctgtgctc aaatagcaaa tgtcaggctt caatgtagct | 13140 |
| agaaggttct agaatgatta agtttccaag gctgaagagc ttccctgttt gcctttcact | 13200 |
| tccctggaga ggtcgttgtg tgttccggag tctgcaaggt gcctttggtg atgcgggtgg | 13260 |
| ttcatctcgg gagattccgc ctggaggacc caagttcaag ccctgcctga gctacagagt | 13320 |
| gactttcagg tcttctgcgc aattcagtga gacccagtct acaaataaaa agtaaaaaga | 13380 |
| aggctgtgga tggaactcgg tggtagagtt ctgggtttac tccctagagg aggggagaag | 13440 |
| gaggaggagg gaggaggaag aggaagaaag aagaagagaa gggaagagga gaaggaaggg | 13500 |
| agggaagggg ctgacaagaa gagagaagag ggagggaggg gagggaaagg aaggggaaag | 13560 |
| gaagggaggg aaggggctga caagaagaga gaagagggag ggaggggagg gaaaggaagg | 13620 |
| ggaaagaaga gaagggtaag aagaaactgt tccaatggtc tgggccacag agtgatggcc | 13680 |
| ttttgtggtg atcagctgta atccttgatt tgacacaacc tagaatctgg gaagcgagtt | 13740 |
| tctgtgaagg agcattcaca ctggctggcc tgtgggcgtg catgtgggag actgtcataa | 13800 |
| ttaggttcat taatacagga agtcccagcc cactacaaat ggcttcgttc catacccaag | 13860 |
| agatgctaac tgtagacggt tggagaaagc aagcaagctg tggatacccc acgctctttc | 13920 |
| acctcggctc ctggggggtg ggtgcactgt gtctcttggt attttaaagt cctgccttga | 13980 |
| cgtccctgct gtgacagact gtaactggaa ttgtgagctt tagtccttta gttttctacg | 14040 |
| ttggtttttc tcaggatatt ttatcgcagt aacagaaaca agaccaggac acttgatctc | 14100 |
| ctctgatcaa cactgaagag ttacaaaaca ggctgaggaa acaaactttc ttctccctct | 14160 |
| cccccttctg tccctcccct tccttctcgc tccctccctt gccccctctc tccctgtctc | 14220 |
| tgtctctgtc tctgtctctg tctctgtctc tgtctctgcc tctcccctcc cctcccctcc | 14280 |
| ctctgtctct gtctctgtct ctgtctctgt ctctgtctct gtctctgtcc ctttctcctc | 14340 |
| tatctcctaa atggctggag gccatgctag ctcaatgttg aactttgaac acgtatttag | 14400 |
| gaaatctttg ttcttaacag ttctgaagtg ctgaagtggt ggtttagtct ctcggcctga | 14460 |
| caagctcact tcctctcact ctgtcttaat gaccaaatct gccatttccc taaaacagca | 14520 |
| caggctccag ctccaggttg ctccggagcg gag | 14553 |
| EXAMPLE 12-CHO Stable Site 2 Sequences-U.S. Pat. No. 9,816,110 | |
| <211> 4001 | |
| <212> DNA | |
| <213> Cricetulus griseus | |
| <400> 1 | |
| (SEQ ID NO: 11) | |
| ccaagatgcc catcaactga ttaatagatg ataaaattat tgtacatttc agtgtaatat | 60 |
| tattcagttt ttaagaaaaa tgaaattatg taataagcat gtaaatggat atatcttgaa | 120 |
| acaaccattc cccattatat tacctaaaca ttgaaagtcc aaaatcatat gatcttttta | 180 |
| gtggatctac taatcttttg ctatatgtat tttattgaac tacccatgga tgtgagataa | 240 |
| ttggtaacaa cagcacatgg gagagcatgg gatcattcaa ggaagattag agagaatgca | 300 |
| ttttttagga gataatggag gagcaataga aaggattaaa tgaggttact gatgaaagtg | 360 |
| atggttagag aaggcaatat gaggagggat aactagcact tagggccttt tgaaaaagac | 420 |
| atagagaaaa tactattgta gaaacttcct ataattggtg tatagttata tacaccaaag | 480 |
| agctcagatg gagttaccct ataatggaaa tattaactac tttttatcac tgtgataaaa | 540 |
| catcctgaac agagcaacat agattgggaa gcatttactt tggcttacag ttctaacggg | 600 |
| ataaaaattc atgatgaaag aatgaatatg tcagcaaaca gcagtagcaa tggcctgaga | 660 |
| agcaggtgag agctcacatc ttgaagtgta agaatgtagc agagagaaca aactgcaaat | 720 |
| gaccagaaaa tgcttttgga tcagagccca tacccctctg actgacttct ccagaaattc | 780 |
| tgaacaaata aaactcccca aacagagcca taactgaagg tccagtgtct gagactacta | 840 |
| ggggtatttc ttattcaaac cactacaatg gggtgggggg agcaatcctc caagtaggca | 900 |
| ctacacacag acaaataaaa actctagtaa ctggaatgga ttgacttatt tgaattactt | 960 |
| gccagtggag ctacatagag cacaattatt gtatttaaat taccctttat gatcttacaa | 1020 |
| aacttgacag taagatcata ttgctaaaga aaccacatat ttgaatcagg gaacatggtg | 1080 |
| atatctagtt gttcttcaac tggaaacttc atgctttctg cccagcattc atgttgctgg | 1140 |
| aaagagcaat gtacactacc agtgtagaaa ttaaatcatc aatcttatca agatgtggat | 1200 |
| cctataagtt acaataaaaa ttagcctgat aagatatccc caccagaaga atattcacat | 1260 |
| aaatgctatg ggagcaacaa gctattttct aaattagctt taatcctatt ctacaagaga | 1320 |
| gaatccatat ctagaatagt tatagggatc aagaacccat ggcttgattg gtcataggcc | 1380 |
| caatgggaga tcctaatatt attgttctac aaaatgaaaa taactcctaa tgacttgttg | 1440 |
| ctgcagtaat aagttagtat gttgctcaac tctcacaaga gaagttttgt cttacaataa | 1500 |
| atggcaatta aagcagcccc acaagattta tatcataccg atctcctcat ggcctatgca | 1560 |
| tctagaagct aggaaacaaa gaggacccta agagagacat acatggtccc cctggagaag | 1620 |
| gggaaggggg caagacctcc aaagctaatt gggagcatgg gggaggggag agggagttag | 1680 |
| aagaaagaga aggggataaa aggagggaga ggaggacaag agagagaagg aagatctagt | 1740 |
| caagagaaga tagaggagag caagaaaaga gataccatag tagagggagc cttgtatgtt | 1800 |
| taaatagaaa actggcacta gggaattgtc caaagatcca caaggtccaa ctaataatct | 1860 |
| aagcaatagt cgagaggcta ccttaaaagc ctttctctga taatgagatt gatgactacc | 1920 |
| ttatatacca tcctagagcc ttcatccagt agctgatgga agcagaagca gacatctaca | 1980 |
| gctaaacact gagctagttg cagacaggga ggagtgatga gcaaagtcaa gaccaggctg | 2040 |
| gagaaacaca cagaaacagc agacctgaaa aaaatgttgc acatggaccc cagactgata | 2100 |
| gctgggagtc cagcatagga cttttctaga aaccctgaat gaggatatca gtttggaggt | 2160 |
| ctggttaatc tatggggaca ctggtagtgg atcaatattt atccctagtt catgactgga | 2220 |
| atttgggtac ccattccaca tggaggaatt ctctgtcagc ctagacacat gggggaggtt | 2280 |
| ctaggtcctg ctccaaataa tgtgttagac tttgaagaac tcccttgaga agactcaccc | 2340 |
| tccctgggga gcagaaaggg gatgggatga gggttggtga gggacaggag aggaggggag | 2400 |
| ggtgagggaa ctgggattga caagtaaatg atgcttgttt ctaatttaaa tgaataaagg | 2460 |
| aaaagtaaaa gaagaaaaga aaacaggcca aaagattata aaagacagag gtggtgggtg | 2520 |
| actataaaga aacactatta tctaaataaa aacatgtcag aagcacacat gaacttatag | 2580 |
| tgtttatgaa agtatgtata ataactacat aatctcaagc caagaaaaaa atatcatctt | 2640 |
| tcagtgatga aggtgatttt atttctccca gaattaaagc caaagaccta atgaaagtaa | 2700 |
| ttatcttcaa aaggttgaaa atacatactt tccaatacac agatctgcct agaaatctca | 2760 |
| tcttcacaat acacatgatg ctcaattgaa ttccattcaa tgttacagtt tagataaaca | 2820 |
| gtttgtagat aaactcacaa tgtatcattt ctttttattt tttgaccaaa cagcttctca | 2880 |
| tctgttattc agaataattc ctcgatggca ggatatccat cccaattggg ggaaggggag | 2940 |
| aatttgaaga aaacctagac cacatacata tttgccattg ggaaacaaag tctaaaatga | 3000 |
| tgttgttcac atcttctcta ctagtcctct ccccgtccca aagaaccttg gtatatgtgc | 3060 |
| ctcattttac agagagagga aagcaggaac tgagcatccc ttacttgcca tcctcaaccc | 3120 |
| aaaatttgca tcattgctca gctctgccct tctcatatga cagttacaag tcaaggcttc | 3180 |
| caaagtccct ctgtcatgtt tggtgtcaat agtttataca gatgacttca tgtcttcata | 3240 |
| tctaatgtct tatatagatt aatattaaac aatgttattt ctctaaccac attttaaatt | 3300 |
| aatttaaaaa tccattaatt gtgtctataa aatgcagaca gagtgctgag acacaatata | 3360 |
| agcctgatga tctgaatttg aaactcacac ccaccacatg gagaatcaac ttccaaaaat | 3420 |
| tttcctatta cttccacact tacaccattg tacaaacaca ataataatga acaaaatgaa | 3480 |
| atgaaataaa aaattaagtc tctgtaggta atgctactgt gcagcaaaag taaaaatggc | 3540 |
| agcttaagct tgctttatgg ttacacttta ccatcttcca ttaattataa ggacttcaat | 3600 |
| catggcagaa ctatgctgtt attgtctcag tgtaacctaa ccaggtgttc cagatgttct | 3660 |
| taatgtggac acctaaacta tttgatattt gggttaagat ctttccctct ttcagaagaa | 3720 |
| acctcaggac agagggaatc ttgtctttta attttgagtc tgtagacttt ttccatttca | 3780 |
| aatatacatg aaacaagtga tgaagaaaat taatcaaaag gtgggaattg caatgatatt | 3840 |
| aggttcaata ttaagcttca atattatcat ggaatcgcct gttatacact gagtgtttgg | 3900 |
| caataaggga tttttagaag aaggagtttt tattctcaac aggttcctta agtttagctc | 3960 |
| aaataaatct aagcaatcca ctctagaatt aaatagtttc c | 4001 |
| <211> 14931 | |
| <212> DNA | |
| <213> Cricetulus griseus | |
| <220> | |
| <221> misc_feature | |
| <222> (2176)...(2239) | |
| <223> n is a, c, g, t or nucleotide is missing | |
| <400> 4 | |
| (SEQ ID NO: 12) | |
| catgtacact tatgcaagta tgatatggcc caacacagta ttttacacca atttttatct | 60 |
| ataaaatata catgtacatc aaaatatatt attaataata acatcattat tctttctttc | 120 |
| caagtaataa acacatacac tgaaattttg gttcttgtgg ataattttaa tgaaacagga | 180 |
| aatgcaaatt tatcttagca tctttacttc actttctttg catagataac cagtaatcac | 240 |
| attgatggat catgtagtga aatgtatttt taggtatcta aggaattttg gcttcgtttt | 300 |
| gtgcttgttg acactgaatt ctattcctaa caacagtgtg taaggattct gtctgatttc | 360 |
| ttttaccagt atttgtccat ttgcattttc tttattattc atggctgctg ttctagaaag | 420 |
| tggaaggtag tgtgtcaagt ctgtttaaca tctttccctg atgatcagtg tcttaacacc | 480 |
| tctctgagta catgttggcc aatgtcgttt ctagacccat ctattcttgc ttgacttatc | 540 |
| ctggtacatg cctgccaaga aatttctcct catcctttct gtctcttcac tgatttactt | 600 |
| gatgtgtgga tttcacattg atcatatgga aatagaagat acaattttct ttattcacag | 660 |
| tttggaagac tttcaatctc atagatcatc attatttttt gctactgttc cctatgctat | 720 |
| ggtgaaattt ccatttgaat aattgcttaa acaattaaca agaaagaatc tatttttact | 780 |
| tgcaataact tccatttcag aacatttact acactgttac tatatccaaa aactagtttt | 840 |
| atatatcatg tgagaaatga ctaattcata atttggccat gacatttttt tcagaaacag | 900 |
| aaaaagtgac caatacatac acaatgctat aaatattaag acttcagcaa attaaatatt | 960 |
| tattcatgat atcacataaa attcatttat tatgttttat ttaaatgtgt ttttaaaaca | 1020 |
| gtggtatcac taaatattaa gttagatgtg tttatgtgct taatgaattt atattttaga | 1080 |
| atgttataag ttgtatatag tcaaatatgt aataaatttt attttttagg tctttctcat | 1140 |
| taaggtattt taattttggg tcccttttcc agagtgactc tagctcatga tgagttgaca | 1200 |
| taaaaactaa acagtacaaa atgtacattg cattcagtat tgcacttgat ctttgcactg | 1260 |
| aagtttgagt cagttcatac atttagtact tcggaagtac attaagctaa ctttcattgc | 1320 |
| tctggcaaaa tgctcgataa gataagagtc tattgtggaa agccatggca gcaggaaagt | 1380 |
| aagactgctg atgatgttta atccatagtc aagacgcaga aggagatgaa tgctggtatc | 1440 |
| caacattttt tgctgttcat tttctctaga accctagtcc ataaagatgt atgacttgca | 1500 |
| ttcaaaatgc gtccccttca gttgttcaac ttttctgtaa atatcctttc aggcatgtct | 1560 |
| agaagattgt ttcgcaaata cttctcaatc cattcaagtt gatagtgcag attaatcact | 1620 |
| gcagaataaa agcctgtaac ttggctcacg tcccaaggaa tatgcacact cctgacacat | 1680 |
| caataagtaa atcaaagtgt agcttttgcc tttaacattg ccagacttat gtaatgttct | 1740 |
| gcacgttctt cctccatcac tttttattct aatggtgttt ccttgacatt gaatcacgct | 1800 |
| gtggaagctg cttagaatta acattgaaat ctactgatat atttatgatg cagcaattta | 1860 |
| gatttactat tttacttaga attttttata attgagagaa tataatattt tcacagttat | 1920 |
| ctatctgctg taaatagagg attttaaaaa aaatctctat aacttttttt tacaacacac | 1980 |
| agtaaaatta agttaaaatt taataaagtc actatgttga tttcaaagtg tgctacgccc | 2040 |
| acggtggtca cgcaggtgta gcagaagatg ccactaaggt gggctaaggc cgatgggttg | 2100 |
| gggtctgcgc tccctggaga tgagccccag gcggttccct ggcaatcagc tgcgatcatg | 2160 |
| atgcccgatg agccannnnn nnnnnnnnnn nnnnnnnnnn nnnnnnnnnn nnnnnnnnnn | 2220 |
| nnnnnnnnnn nnnnnnnnnc tgggtgactt tatggaaaga atttgataga tttcatgatg | 2280 |
| tagaagaatt ttattaggct tattttacag gagactaaga ccctgggacc taaagatatc | 2340 |
| tgggtcctga gaatcaggaa atgggtagag acgtggttga tggtatgaga cagattttag | 2400 |
| agaactctta gatcatgggc aatgaccgca atctgatgct tagaatagat catctataaa | 2460 |
| caattatgct gttctttttc tttctgttgt atgatctgat gatgtagccc ccttgccaag | 2520 |
| ttccctgatc ccccttgcca agttccctga ttgtaacagt atataagcat tgcttgagag | 2580 |
| catattcaac tacattgagt gtgtctgtct gtcatttcct cgccgattcc tgatttctcc | 2640 |
| ttgagccttt tcccttgttc tccctcggtc ggtggtctcc acgagaggcg gtccgtggca | 2700 |
| aaagtgtata aatgttctaa aacatttgaa ctctaaaaca tccaaaatga aaaattaaaa | 2760 |
| taaataaaca tgaaaattaa aatatattag ctgctaaaag ttaaacaata ctatataata | 2820 |
| ttttgttatt agaattcaaa atcacattag ttggatttaa tttgaacatt gcattctttc | 2880 |
| aataataatt tcaataaaaa aagtttcccc atgatagtag aaaataataa catatgtatc | 2940 |
| tatctattta tttaactaca catatatagc atttgtttca actaaaataa atgaatgagc | 3000 |
| aaagcaccta agtaattggt gtctattata tttatgaagc caatagtttc aaataaatta | 3060 |
| tcatgcataa ggaggtattg caaatgttaa accttttttg aaacagatat tcccagttac | 3120 |
| agaaattata atttctaatc tttcctataa gtagaatgat gataattaat ataggccatt | 3180 |
| tgtaaataat gttcagatta aaatattctc tatttcacta gagaagaatg atattaaatg | 3240 |
| tattatattt tatttcccat tttgtttgca ccactattct atatccctca gcagtttaaa | 3300 |
| tttgtttcac catatgtgtg tgtgtttgta tcttaaatat ggcactaaaa ttagaataat | 3360 |
| ttaatataaa tctttaggag aaaagatatt gaattatttt atgttgatag gaaaatatct | 3420 |
| tttaattgtc caagaatact ttttcttcta ttttaggact gatcagaccc aggactaata | 3480 |
| ttttatatgt actaattcta tgtaccaaaa tatgttatta tctcatgaat tctgtctcaa | 3540 |
| tattgaggta ataaaaatag tccatcatga actttaaaat taaaataatg attaattaat | 3600 |
| ttttattcat attttgtttg tatgaatggt tatacatcac atgtgtgcct ggtgactgtg | 3660 |
| aatgtcagga gaaggtatga aagccactgg aattggaata agagataata tttgagatgt | 3720 |
| tatgtgggtg ctgagaatta gacgcaagcc atcttcaaga atagccagca tactatacca | 3780 |
| ctgagtaatc cattcatccc tcaataatta tctttgtaga cagtaaatat atttctaaac | 3840 |
| tataaatgac cagaaaaatt aatgtattat taatgaagac attcatctca tgtgacacac | 3900 |
| ttcacctgtc taaatcagta acactctctc cactaattaa gattttctaa gtgcatgaca | 3960 |
| cttactattt ctaaagctgt ccaatggggg ccagtcccca gtcagcaccc agtgagataa | 4020 |
| tccatgaatg catttatatc ttaggaaaaa ttcttatcta tgtagtattt agaacatttt | 4080 |
| catgtgaggg gataaacaag gaagcacaga tgctttctga tagaaacttt ctctttaatt | 4140 |
| catctagaaa aaaaaaacct ctcaggaaaa tctctcttgc tctcctccca atgctctatt | 4200 |
| cagcatcttc tccctactta attctagatc tttttctcta tgcctccttg ctgctgccct | 4260 |
| gctggctctg ctctatgcct ccccatgtca cttttctttg ctatctcacc gttaccttct | 4320 |
| ctgcctcact ctctgccttc ttctctgctt ctcacatggc caggctctgg acaattatag | 4380 |
| ttatatgtta cattctcata acacatgata tgtcacatag tttctctcag gctagggata | 4440 |
| tcacaatgac tggccaatga gcaagtggcc ttgcatgtag ctctaagttg gtgatggttc | 4500 |
| ccagacagta agtagccatt tggttgaaat ttgaggttgg gtagtacatg aagactgaat | 4560 |
| tttcttcaaa ctctggcctt gaaatagtaa aacaacacct atgaaaatga cgacctgtat | 4620 |
| ttgtctttag aggcaaccac atattgtctg cagggcctgc tttgaatttg ctctgaagtt | 4680 |
| agcttgtttg tgtaaaagga agaatcctat atcagcctga gaaatgtaaa atatcctagc | 4740 |
| atttcaagtc atcaaaatta tatggagagt ataaatcatc cttctgacta ttcatagtca | 4800 |
| tatttgtgtc caccaagtat aaaacacact accaaagggc tgtggaaaaa atcgccataa | 4860 |
| ctgttcttat tagggaggca tagcagtggt acctgaggaa gttacagcaa caaccagtca | 4920 |
| tccagtcaat aaccccatgg ctttgccact tggaggtacc caataatgtt tggctttgcc | 4980 |
| gagtaggact ccaacaaatt cagagggtca atttttaaat gctggttgtc actgctgaac | 5040 |
| agtcccattg ccctctgcat aattccacaa tggaaagctt tttacactga ttgccaatca | 5100 |
| ttaaacagcc tactcagcat aaacaggtat gatattattc tccattttgt tacattacta | 5160 |
| gatgaattcc tatttcttcc tacaatagtg gaactgaaaa aagatacaca atcatactac | 5220 |
| ccctctacta atcttatgac ttatatcatt tcaattttca gaccataatg caaactattg | 5280 |
| accaaaacat gtgaagatga aaaatagaaa tgtagaataa tattacatat aaaaagaaaa | 5340 |
| ggcggactta ttttgtttta tttcttagca tgcatagcaa tacatgattt gaggtttata | 5400 |
| taataaaggg acaataaatc ttcaagaaac ttacccctac tgaattaaaa tattaaagaa | 5460 |
| ggtcacacat ttactcaaat atattagact actgggcaaa tagacatgaa aagtagagtt | 5520 |
| aatattgagg taggccttct gtgaaatgtc taaggaaatt atgtttcata cagtgtgtaa | 5580 |
| ccaagtggga atcatatcag aaagcagtca aaagcttata ttacaagtaa cagatgcttg | 5640 |
| gttatatgac ctcccagagc ttgactgtct atacacaaaa agtggtgtta ataaaactgt | 5700 |
| aatttgggct atgttttttt aaatggcttc accaacatga aaggaaggga atgagcatgt | 5760 |
| catggatgct tagagattat gcttccagca agaagaattg agctttggct cttattacag | 5820 |
| aaacatgaca aggtgtgagt tttatttatt agaaattata taatatttta agctggggac | 5880 |
| taaaaatttt attgaaacaa acaggcaagg gataggcatg tactagaagc aaaaatagga | 5940 |
| tgtcaatgct gtaatgttat tttttggacc aaaatagtat ttcctataga aatgacaatg | 6000 |
| atcttaggtt attattcttc ataaagatga caagttcaca agatatccta gttcattaaa | 6060 |
| atcgttttag tcatttaata gagtgctgtg atagattaca caaaggaaag cacttacgat | 6120 |
| gagaaataat gatatccaca attattttct taattcttag aaacattcta ttgttatatc | 6180 |
| tcaatctcag aagccactta ttgctttatt attgaaacat atgaaattgt aagttatata | 6240 |
| ttgtctatgg tgacatttca aagaacatgt gacgtacagt gtagcacaga taaagaacat | 6300 |
| aactgcagct gaatcagtaa ctaaacttac atacattaaa tctgccatgt tggcaacagt | 6360 |
| gtgtgcacta ccaaaggatg tactaatgct cacgacactc ccctatgtca ccctttgttc | 6420 |
| atcattacat cataggtcta ttttgtttgc ttttgaaatc tagaccaagt cttttgtgtc | 6480 |
| tttccaagca cagagctcat taatttacct catagacttg ttaaacttct tctggttcat | 6540 |
| caattgaata gaaatactca ctactaatta tgtgagaccc tcccagtacc atagcacatg | 6600 |
| gataattttt acataaaaca tgcatacaag taagattatt cagactgaac atgaatttta | 6660 |
| gagaaatcag gaaggagtat atgggagtgg ttggagtgag actagagaaa tgtaattaaa | 6720 |
| ctataatctc aatacaaaga tctactaagc aaaaaacatg aaacattgtc attcaagtga | 6780 |
| aacatcagtc ttcaaattgg aaagatattt ttactaggaa aatgtctggt agatggttat | 6840 |
| tatctagaaa acacaaaaat tagaaaacgg taaactttaa taaaaagaat aatacaatga | 6900 |
| gactacatga aaagttctta actaatgaaa caaatatctt gaaacttttt tcttaaaagt | 6960 |
| ttaatatcaa taaccatcat ggaaattcaa attaaaacta tttacatatt acccctgaaa | 7020 |
| taataactaa tacccaataa aaataatata aacaaaaaat ggcaatgcat gccatcatgg | 7080 |
| atttgggaga gagaatgttc attgcagttc tgaatggata ctggtgccac cacggtgaaa | 7140 |
| atctctgtat aggtccttcc aaaagctgaa aatagacata tcacaagacc tcccacacat | 7200 |
| ttttcaagca aatacccaaa ggactctacc tgactgcaga gacactttct cataaaatat | 7260 |
| tattgttgat ctattcataa tatctggaaa atagaaacag ccaagatgcc catcaactga | 7320 |
| ttaatagatg ataaaattat tgtacatttc agtgtaatat tattcagttt ttaagaaaaa | 7380 |
| tgaaattatg taataagcat gtaaatggat atatcttgaa acaaccattc cccattatat | 7440 |
| tacctaaaca ttgaaagtcc aaaatcatat gatcttttta gtggatctac taatcttttg | 7500 |
| ctatatgtat tttattgaac tacccatgga tgtgagataa ttggtaacaa cagcacatgg | 7560 |
| gagagcatgg gatcattcaa ggaagattag agagaatgca ttttttagga gataatggag | 7620 |
| gagcaataga aaggattaaa tgaggttact gatgaaagtg atggttagag aaggcaatat | 7680 |
| gaggagggat aactagcact tagggccttt tgaaaaagac atagagaaaa tactattgta | 7740 |
| gaaacttcct ataattggtg tatagttata tacaccaaag agctcagatg gagttaccct | 7800 |
| ataatggaaa tattaactac tttttatcac tgtgataaaa catcctgaac agagcaacat | 7860 |
| agattgggaa gcatttactt tggcttacag ttctaacggg ataaaaattc atgatgaaag | 7920 |
| aatgaatatg tcagcaaaca gcagtagcaa tggcctgaga agcaggtgag agctcacatc | 7980 |
| ttgaagtgta agaatgtagc agagagaaca aactgcaaat gaccagaaaa tgcttttgga | 8040 |
| tcagagccca tacccctctg actgacttct ccagaaattc tgaacaaata aaactcccca | 8100 |
| aacagagcca taactgaagg tccagtgtct gagactacta ggggtatttc ttattcaaac | 8160 |
| cactacaatg gggtgggggg agcaatcctc caagtaggca ctacacacag acaaataaaa | 8220 |
| actctagtaa ctggaatgga ttgacttatt tgaattactt gccagtggag ctacatagag | 8280 |
| cacaattatt gtatttaaat taccctttat gatcttacaa aacttgacag taagatcata | 8340 |
| ttgctaaaga aaccacatat ttgaatcagg gaacatggtg atatctagtt gttcttcaac | 8400 |
| tggaaacttc atgctttctg cccagcattc atgttgctgg aaagagcaat gtacactacc | 8460 |
| agtgtagaaa ttaaatcatc aatcttatca agatgtggat cctataagtt acaataaaaa | 8520 |
| ttagcctgat aagatatccc caccagaaga atattcacat aaatgctatg ggagcaacaa | 8580 |
| gctattttct aaattagctt taatcctatt ctacaagaga gaatccatat ctagaatagt | 8640 |
| tatagggatc aagaacccat ggcttgattg gtcataggcc caatgggaga tcctaatatt | 8700 |
| attgttctac aaaatgaaaa taactcctaa tgacttgttg ctgcagtaat aagttagtat | 8760 |
| gttgctcaac tctcacaaga gaagttttgt cttacaataa atggcaatta aagcagcccc | 8820 |
| acaagattta tatcataccg atctcctcat ggcctatgca tctagaagct aggaaacaaa | 8880 |
| gaggacccta agagagacat acatggtccc cctggagaag gggaaggggg caagacctcc | 8940 |
| aaagctaatt gggagcatgg gggaggggag agggagttag aagaaagaga aggggataaa | 9000 |
| aggagggaga ggaggacaag agagagaagg aagatctagt caagagaaga tagaggagag | 9060 |
| caagaaaaga gataccatag tagagggagc cttgtatgtt taaatagaaa actggcacta | 9120 |
| gggaattgtc caaagatcca caaggtccaa ctaataatct aagcaatagt cgagaggcta | 9180 |
| ccttaaaagc ctttctctga taatgagatt gatgactacc ttatatacca tcctagagcc | 9240 |
| ttcatccagt agctgatgga agcagaagca gacatctaca gctaaacact gagctagttg | 9300 |
| cagacaggga ggagtgatga gcaaagtcaa gaccaggctg gagaaacaca cagaaacagc | 9360 |
| agacctgaaa aaaatgttgc acatggaccc cagactgata gctgggagtc cagcatagga | 9420 |
| cttttctaga aaccctgaat gaggatatca gtttggaggt ctggttaatc tatggggaca | 9480 |
| ctggtagtgg atcaatattt atccctagtt catgactgga atttgggtac ccattccaca | 9540 |
| tggaggaatt ctctgtcagc ctagacacat gggggaggtt ctaggtcctg ctccaaataa | 9600 |
| tgtgttagac tttgaagaac tcccttgaga agactcaccc tccctgggga gcagaaaggg | 9660 |
| gatgggatga gggttggtga gggacaggag aggaggggag ggtgagggaa ctgggattga | 9720 |
| caagtaaatg atgcttgttt ctaatttaaa tgaataaagg aaaagtaaaa gaagaaaaga | 9780 |
| aaacaggcca aaagattata aaagacagag gtggtgggtg actataaaga aacactatta | 9840 |
| tctaaataaa aatatgtcag aagcacacat gaacttatag tctttatgaa agtatgtata | 9900 |
| ataactacat aatctcaagc caagaaaaaa atatcatctt tcagtgatga aggtgatttt | 9960 |
| atttctccca gaattaaagc caaagaccta atgaaagtaa ttatcttcaa aaggttgaaa | 10020 |
| atacatactt tccaatacac agatctgcct agaaatctca tcttcacaat acacatgatg | 10080 |
| ctcaattgaa ttccattcaa tcttacagtt tagataaaca gtttgtagat aaactcacaa | 10140 |
| tgtatcattt ctttttattt tttgaccaaa cagcttctca tctgttattc agaataattc | 10200 |
| ctcgatggca ggatatccat cccaattggg ggaaggggag aatttgaaga aaacctagac | 10260 |
| cacatacata tttgccattg ggaaacaaag tctaaaatga tcttgttcac atcttctcta | 10320 |
| ctagtcctct ccccgtccca aagaaccttg gtatatgtgc ctcattttac agagagagga | 10380 |
| aagcaggaac tgagcatccc ttacttgcca tcctcaaccc aaaatttgca tcattgctca | 10440 |
| gctctgccct tctcatatga cagttacaag tcaaggcttc caaagtccct ctgtcatgtt | 10500 |
| tggtgtcaat agtttataca gatgacttca tgtcttcata tctaatgtct tatatagatt | 10560 |
| aatattaaac aatgttattt ctctaaccac attttaaatt aatttaaaaa tccattaatt | 10620 |
| gtgtctataa aatgcagaca gagtgctgag acacaatata agcctgatga tctgaatttg | 10680 |
| aaactcacac ccaccacatg gagaatcaac ttccaaaaat tttcctatta cttccacact | 10740 |
| tacaccatty tacaaacaca ataataatga acaaaatgaa atgaaataaa aaattaagtc | 10800 |
| tctgtaggta atgctactgt gcagcaaaag taaaaatggc agcttaagct tgctttatgg | 10860 |
| ttacacttta ccatcttcca ttaattataa ggacttcaat catggcagaa ctatgctgtt | 10920 |
| attgtctcag tgtaacctaa ccaggtgttc cagatgttct taatgtggac acctaaacta | 10980 |
| tttgatattt gggttaagat ctttccctct ttcagaagaa acctcaggac agagggaatc | 11040 |
| ttgtctttta attttgagtc tgtagacttt ttccatttca aatatacatg aaacaagtga | 11100 |
| tgaagaaaat taatcaaaag gtgggaattg caatgatatt aggttcaata ttaagcttca | 11160 |
| atattatcat ggaatcgcct gttatacact gagtgtttgg caataaggga tttttagaag | 11220 |
| aaggagtttt tattctcaac aggttcctta agtttagctc aaataaatct aagcaatcca | 11280 |
| ctctagaatt aaatagtttc ctaagggcac agctatgaat agagctcaat ttacatataa | 11340 |
| aattttgttc accatttatg tcattccagt tttcattagt acaaggaaaa tacaaaatat | 11400 |
| ttagatgtca atatcaagtg aatagttcat ctcctttttt aatatatatc acctaaatca | 11460 |
| ccattttctc agaaaaatct ggcctgaagt tctgtctgga acttcaacat gaaaaatatg | 11520 |
| cacagcttgc tattataaat cctagttgat ttttaagatt catgtctggt gtctgactca | 11580 |
| gaggggccag aggctagaca aatatttttt gaatcttcat tgtgaagatt tttaatgatt | 11640 |
| attttaatat aaataacaaa gatgatggat aatgtaactt tgtacagttc atagacgctg | 11700 |
| aactactttg tgcttaaaat gttagttccc tatcataaat gataggtgat aagtgtatgt | 11760 |
| ttaatacttt ccctctgagc tatattcatg tactagagaa ttattttaaa catgaaaaga | 11820 |
| ctgtgtttat agtctcagct cctgagaact ggtccaacct taggcaggtg aatgccagga | 11880 |
| gcaacgtttt tcttctacag aggatgcttt gctgccaagc aacctggttg tgtggaaatg | 11940 |
| ttcctttttt aatcaagttt aaagggtctt catcatgctg ttgctccaca tattttcagg | 12000 |
| ttagagcttg gtccttggag tattatcttt taccagaaaa ttcatagtat tctttcaata | 12060 |
| actaacaact aaacttttcg ataaaaaaga attggaattt caattttaaa gcctgagtaa | 12120 |
| aattcttgtg aatcaggata ttttatttta agtcttatct tttaaaaagt tattttattt | 12180 |
| tttaaaaaat tataatatac tttcataatt tccctccttc acttttcttt acaaacactt | 12240 |
| ctatagatca ccatgtgttt ttttttttac atttatggcc tctttctgtt cattgttatt | 12300 |
| acatacaaat agtcttgcct atagaagaac accacaattt gttacctgat aacaaattat | 12360 |
| caacccttaa aacctacaaa ctattgatat tactgaaaag actatactta tagatgtaaa | 12420 |
| gatatatgtg tgtgcacata tatagataca catatatgta ggatttttaa ttttagattt | 12480 |
| tagacatcaa aattatttat atgactgaga aactagacac tataaatgag cattcagtat | 12540 |
| tcaacaccgt gattttagat attgtcacaa tgacagaaaa ttttcttata gaaaatttta | 12600 |
| agttttgtga ttgctctgtg cacttagtga agtctcacag aaaaagaatc atagtatttt | 12660 |
| tagtttataa taaaaagtac atataattaa aatggttggc acaaaacaac atttgagcat | 12720 |
| ttttcctatt tactatcaag tagtatcatt ttgaaataat aatttgacta gtttcaaaaa | 12780 |
| tgaaaacaaa atttaaacta aatgcctaat ctagcctgat aacattttta tgaatgaaat | 12840 |
| tattcaatag tcttatcaat taggggccca aaacttttcc taaaataaaa cttttaattt | 12900 |
| ttttccattt ttatttaaat tagaaacaaa attgttttac atgtaaatca gagtttcctc | 12960 |
| accctcccct tctccctgtc cctcactaac accctacttg tcccatacca tttctgctcc | 13020 |
| ccagggaggg tgaggccttc catggggaaa cttcagagtc tgtctatcct ttcggatagg | 13080 |
| gcctaggccc tcacccattt gtctaggcta aggctcacaa agtttactcc tatgctagtg | 13140 |
| ataagtactg atctactaca agagacacca tagatttcct aggcttcctc actgacaccc | 13200 |
| atgttcatgg ggtctggaac aatcatatgc tagtttccta ggtatcagtc tggggaccat | 13260 |
| gagctccccc ttgttcaggt caactgtttc tgtgggtttc accaccctgg tcttgactgc | 13320 |
| tttgctcatc actcctccct ttctgtaact gggttccagt acaattccgt gtttagctgt | 13380 |
| gggtgtctac ttctactttc atcagcttct gggatggagc ctctaggata gcatacaatt | 13440 |
| agtcatcatc tcattatcag ggaagggcat ttaaagtagc ctctccattg ttgcttggat | 13500 |
| tgttagttgg tgtcatcttt gtagatctct ggacatttcc ctagtgccag atatctcttt | 13560 |
| aaacctacaa gactacctct attatggtat ctcttttctt gctctcgtct attcttccag | 13620 |
| acaaaatctt cctgctccct tatattttcc tctcccctcc tcttctcccc ttctcattct | 13680 |
| cctagatcca tcttcccttc ccccatgctc ccaagagaga tgttgctcag gagatcttgt | 13740 |
| tccttaaccc ttttcttggg gatctgtctc tcttagggtt gtccttgttt cctagcttct | 13800 |
| ctggaagtgt ggattgtaag ctggtaatca tttgctccat gtctaaaatc catatatgag | 13860 |
| tgatgtttgt ctttttgtga ctgggttacc tcactcaaaa tggtttcttc catatgtctg | 13920 |
| tggatttcaa tagcacaaac aacatacagt atcttggggc aacactaacc aaacaagtga | 13980 |
| aagaccagta tagcaagaac tttgagttta aagaaagaaa ttaaagaaga taccagaaaa | 14040 |
| tggaaagatc tcccatgctc tttgataggc agaatcaaca tagtaaaaat ggcaatcttg | 14100 |
| ccaaaatcca tctacagact caatgcaatc cccattaaat accagcacac ttcttcacag | 14160 |
| acctgaaaga ataatactta actttatatg gagaaacaaa agacccagga taggccaaac | 14220 |
| aaccctgtac aatgaaggca cttccagagg catccccatc cctgacttca agctctatta | 14280 |
| tagagtaata atcctgaaaa cagcttggta atggcacaaa aatagacagg tagaccaatg | 14340 |
| gaattgagtt gaaaaccctg atattaaccc acatatctat gaacacctga ctttgacaaa | 14400 |
| gaagctaagg ttatacaatg taagaaagaa agcatcttca acaaatcgtg ctggcataac | 14460 |
| tggatgctgg catgtagaag actgcagata gatccatgtc taatgccatg cacaaaactt | 14520 |
| aagtccaaat ggatcaaaaa cctcaacata aatccagcca cactgaacct catagaagag | 14580 |
| aaagtgggaa gtatccttga ataaattggt acaggagacc acatcttgaa cttaacacca | 14640 |
| gtagcacaga caatcagatc aataatcaat aaatgggacc tcctgaaact gagaagcttc | 14700 |
| tgtaaggcaa tggataagtc aacaggacaa aatggcagcc cacggaatgg gaaaagatat | 14760 |
| tcaccaatcc tatatctgac agagggctgc tctctatttg caaagaacac aataagctag | 14820 |
| tttttaaaac accaattaat ccgattataa agttgggtag agaactaaat aaagaattgt | 14880 |
| taacagagca atctaacttg gcagaaagac acataagaaa gtgctcacca t | 14931 |

EXAMPLE 13-
Guide Sequences for AAVS1-Like Region
Sequences in CHO (The below guides can be
sense guide sequences or antisense guide
sequences)

| | SEQ ID NO |
|---|---|
| CCCCGCTGGCGCCGGGATCGGGG | 13 |
| GAGTCGAGCACCGCTCGGGCAGG | 14 |
| TTCCCCGCTGGCGCCGGGATCGG | 15 |
| GTGTGCGGAAGACGCCGCCGGGG | 16 |
| CGGTGACAGCGCGGATGACAGGG | 17 |
| CAGCGCGGATGACAGGGGCGAGG | 18 |
| GCCGGCGTCCGATTCCCCGCTGG | 19 |
| CGTGTGCGGAAGACGCCGCCGGG | 20 |
| GAGGCGCTCCACCGTCTGTTGGG | 21 |
| GTCCGATTCCCCGCTGGCGCCGG | 22 |
| GACCCCGGGGGCCCCGATCCCGG | 23 |
| CGGCGTCTTCCGCACACGGATGG | 24 |
| TCGAGCACCGCTCGGGCAGGCGG | 25 |
| AGCTCACGCCGGCCCCATAAAGG | 26 |
| CATCGTCCTCTATATATAGCAGG | 27 |
| AGAGGCGCTCCACCGTCTGTTGG | 28 |
| GGTCGGCTGCGCGAAGCATCAGG | 29 |
| TGCTTCGCGCAGCCGACCCCGGG | 30 |
| GGCCCCGATCCCGGCGCCAGCGG | 31 |
| TCCGATTCCCCGCTGGCGCCGGG | 32 |
| TCCCGGCGCCAGCGGGGAATCGG | 33 |
| TGGTGGAGTCGAGCACCGCTCGG | 34 |
| CAAGATGGTCCTCACTCTCGGGG | 35 |
| GCTTCGCGCAGCCGACCCCGGGG | 36 |
| GTGGAGCGCCTCTTCTCCAGGGG | 37 |
| GACGTGTCAGCCTTCCAGGTGGG | 38 |
| GCCAGCGGGGAATCGGACGCCGG | 39 |
| TCTCCCCGTCATCCAAAAGCTGG | 40 |
| GGTGGAGTCGAGCACCGCTCGGG | 41 |
| GCTGCCCAAATATAGTCCATGGG | 42 |
| ATGCTTCGCGCAGCCGACCCCGG | 43 |
| GCGGTGACAGCGCGGATGACAGG | 44 |
| ATGCTCGGGGGCCGCTGACCTGG | 45 |
| TTGTATTGCCGGGATCCTTCTGG | 46 |
| TCCCCGCTGGCGCCGGGATCGGG | 47 |
| CTCGACTCCACCAACGCCGACGG | 48 |
| GGTGGCAAGATCACCAAAAGGGG | 49 |
| GGCGCTGATGCCGTCGGCGTTGG | 50 |
| TCCACGAGCATCCTAGCAAGAGG | 51 |
| AGGCTGACACGTCAGGCCTGAGG | 52 |
| GGGTGTAAGCCATCCGTGTGCGG | 53 |
| AGGATCCCGGCAATACAAGATGG | 54 |
| GGATGGGGCCCAACAGACGGTGG | 55 |
| GAGGACCATCTTGTATTGCCGGG | 56 |
| AGTCGCCCAGGGTCCTGGTGGGG | 57 |
| GGCGGGAGGAGAGTCCCACCTGG | 58 |
| ACCTACCCCACCAGGACCCTGGG | 59 |
| TCAGCGTCTTTGACCAGTCCAGG | 60 |
| CGTCCCGCCGCCTGCCCGAGCGG | 61 |
| GTCCCCGGGATCCCCGGGGTCGG | 62 |
| AGCACCGCTCGGGCAGGCGGCGG | 63 |
| CGGTGGAGCGCCTCTTCTCCAGG | 64 |
| GCCCCGATCCCGGCGCCAGCGGG | 65 |
| ATCGGGGCCCCCGGGGTCCCCGG | 66 |
| ATTCTCGGCTCATCCCCTTTTGG | 67 |
| ACCACCCCATGGACTATATTTGG | 68 |
| TAGCAAGAGGACGACAACCCAGG | 69 |
| GCTGATGCCGTCGGCGTTGGTGG | 70 |
| TACAAGATGGTCCTCACTCTCGG | 71 |
| TGCCGGGATCCTTCTGGATTCGG | 72 |
| CCCAAATATAGTCCATGGGGTGG | 73 |
| TACCTGTAGAATGGGACCAGTGG | 74 |
| TCTTGCTAGGATGCTCGTGGAGG | 75 |
| ATGCCAGCTTTTGGATGACGGGG | 76 |
| ATAGTCCATGGGGTGGTAGGTGG | 77 |
| CAGGACCCTGCTATATATAGAGG | 78 |
| GGGGCCGGCGTGAGCTGTGTGGG | 79 |
| ACCTGGAAGGCTGACACGTCAGG | 80 |
| CAGCGGACAGCACGGGTCACAGG | 81 |
| CGCCGGGATCGGGGCCCCCGGGG | 82 |
| GCGCCGGGATCGGGGCCCCCGGG | 83 |
| GCCGGGATCCTTCTGGATTCGGG | 84 |
| GCTGTCACCGCTCTCCCCGGCGG | 85 |
| GGTGACAGCGCGGATGACAGGGG | 86 |
| GAAGACGCCGCCGGGGAGAGCGG | 87 |
| AAGGGGTACACTGCCTTGGAGGG | 88 |
| CTTCTAGAACCTACCCCACCAGG | 89 |
| GGGGCCGCTGACCTGGTGCAGGG | 90 |
| TCCCGAATCCAGAAGGATCCCGG | 91 |
| TCACAGTGTCTGAGTCGCCCAGG | 92 |
| GCTAGGATGCTCGTGGAGGTGGG | 93 |
| CTCGTGGAGGTGGGGAATAAAGG | 94 |
| TGATCTGTGCCCCGAGAGTGAGG | 95 |
| TGAATTAATAGGACATGGGGAGG | 96 |
| ACTCTCGGGGCACAGATCACTGG | 97 |
| GACCACTGGTCCCATTCTACAGG | 98 |
| TGAGGACCATCTTGTATTGCCGG | 99 |
| AGAATCCGTCTGTCCTGGGCTGG | 100 |
| TGACGTGTCAGCCTTCCAGGTGG | 101 |
| AAAAGCATCCCGAATCCAGAAGG | 102 |
| TTTTCCCGAGGCCACACTCAGGG | 103 |
| AGCGCCCTGCACCAGGTCAGCGG | 104 |
| TGAGTCGCCCAGGGTCCTGGTGG | 105 |
| CACGTCAGGCCTGAGGTCACAGG | 106 |
| GTTTTCCCGAGGCCACACTCAGG | 107 |
| GAGTCGCCCAGGGTCCTGGTGGG | 108 |
| GAATCCGTCTGTCCTGGGCTGGG | 109 |
| CCGGGGAGGGAGGATGCTCGGGG | 110 |
| GAGCCGAGAATTGAATTAATAGG | 111 |
| GTGACCCGTGCTGTCCGCTGTGG | 112 |
| GGGGGCGTCAAGTCAGAGCTGGG | 113 |
| CGCGCTGTCACCGCTCTCCCCGG | 114 |
| TGCTGCCCAAATATAGTCCATGG | 115 |
| AGTTGAGGAGAAACCTATGGGGG | 116 |
| ATCGTCCTCTATATATAGCAGGG | 117 |
| GAGTTGAGGAGAAACCTATGGGG | 118 |
| AGGGGTACACTGCCTTGGAGGGG | 119 |
| ATAGAGTCCCTCTGGGGACAGGG | 120 |
| CTAGGATGCTCGTGGAGGTGGGG | 121 |
| CGCGGATGACAGGGGCGAGGCGG | 122 |
| AAAACAGAATCCGTCTGTCCTGG | 123 |
| TGCTAGGATGCTCGTGGAGGTGG | 124 |
| GAGTGTGGCCTCGGGAAAACAGG | 125 |
| CGAGGCGGCCCCTGCAGGGCAGG | 126 |
| CTTACACCCCGTGCCTTTCCAGG | 127 |
| CTGCCCAAATATAGTCCATGGGG | 128 |
| AGGGGCAAAGGACCCTCCTGAGG | 129 |
| TCTCACCATAGAGTCCCTCTGGG | 130 |
| AGTGTACCCCTTTGTTCCCCTGG | 131 |
| GTTGAGGAGAAACCTATGGGGGG | 132 |
| CACAGTGTCTGAGTCGCCCAGGG | 133 |
| TCCTCTTGCTAGGATGCTCGTGG | 134 |
| CACATGATCACCAAAGTCCCTGG | 135 |
| AAACAGAATCCGTCTGTCCTGGG | 136 |
| GTGCAGGGCGCTGATGCCGTCGG | 137 |
| CAGCACGGACTTTTTTTGTTTGG | 138 |
| CAGCCGACCCCGGGGATCCCGGG | 139 |
| TTTGGTCAAGATTTTGCAACTGG | 140 |
| TTGGTCAAGATTTTGCAACTGGG | 141 |
| CCTGTCAGAGAGGATGCTCTAGG | 142 |
| TGGGTTGTCGTCCTCTTGCTAGG | 143 |
| CTCACCATAGAGTCCCTCTGGGG | 144 |
| AGACGCTGACACCCTGAGTGTGG | 145 |
| GGTGGAGCGCCTCTTCTCCAGGG | 146 |
| AACAAAGGGGTACACTGCCTTGG | 147 |
| CTGTCCCCAGAGGGACTCTATGG | 148 |
| GTCTCTGACCCCCTCATTTGTGG | 149 |
| GTCTGAGTCGCCCAGGGTCCTGG | 150 |
| GCCTGACGTGTCAGCCTTCCAGG | 151 |
| ATGTCCCACACAGCTCACGCCGG | 152 |
| GGCTGTGCCGCAGGCTTCCAGGG | 153 |
| CACCCCGTGCCTTTCCAGGCTGG | 154 |
| TCACCAAGGTGTCTGCATGGCGG | 155 |
| GAATTGAATTAATAGGACATGGG | 156 |
| AATATAGTCCATGGGGTGGTAGG | 157 |
| CAGGGCATAGTTTTTAAAGCAGG | 158 |
| CGGCATCAGCGCCCTGCACCAGG | 159 |
| GCAGCCGACCCCGGGGATCCCGG | 160 |
| ACAGGACCTGTATTTGAGGTTGG | 161 |
| GGGGCGAGGCGGCCCCTGCAGGG | 162 |
| AACCTACCCCACCAGGACCCTGG | 163 |
| GCACCGCTCGGGCAGGCGGCGGG | 164 |
| TGGAAGCCTGCGGCACAGCCAGG | 165 |
| GAACCAACACTGTGGCCAGGAGG | 166 |
| AGCAGGGTCCTGTTTTCCCGAGG | 167 |
| TGGGTGGCAAGATCACCAAAAGG | 168 |
| AGCCGACCCCGGGGATCCCGGGG | 169 |
| CATGGCAACTTCCATCTCCTGGG | 170 |
| ACAGCACGGGTCACAGGAAGTGG | 171 |
| GGGGGCCGCTGACCTGGTGCAGG | 172 |
| AGGGGCGAGGCGGCCCCTGCAGG | 173 |
| GTGGTCACCCCTGTCCCCAGAGG | 174 |
| TGGGGCCGGCGTGAGCTGTGTGG | 175 |
| GGAAGCCTGCGGCACAGCCAGGG | 176 |
| CCTGAGCTGATCTCCTGGACTGG | 177 |
| ATCCAAAAGCTGGCATTGTCAGG | 178 |
| CTCTCACCATAGAGTCCCTCTGG | 179 |
| GGGGGGCGTCAAGTCAGAGCTGG | 180 |
| GGGTGGAAATCTAAGAGACAGGG | 181 |
| CGGGGAGAGCGGTGACAGCGCGG | 182 |
| AGAATTGAATTAATAGGACATGG | 183 |
| CCATAAAGGAAGTTTTCCACAGG | 184 |
| AGTGAACCAACACTGTGGCCAGG | 185 |
| GTTGGGAGGGAACTCTTGGGAGG | 186 |
| CGGGTCACAGGAAGTGGGGTAGG | 187 |
| GGCCTGGCTAGCCTCAGAGGAGG | 188 |
| GGTAGGTTCTAGAAGGTGACAGG | 189 |
| ACAAGATGGTCCTCACTCTCGGG | 190 |
| CAAGGTGTCTGCATGGCGGGAGG | 191 |
| TGTTTCACTCATCCAGGCAGAGG | 192 |
| TGTGGAAAACTTCCTTTATGGGG | 193 |
| GAGGACGACAACCCAGGAGATGG | 194 |
| GGGTGGCAAGATCACCAAAAGGG | 195 |
| CTGGTGGGGTAGGTTCTAGAAGG | 196 |
| ACTCTTCAGGCCTTTGCAGGAGG | 197 |
| CAGAGGGACTCTATGGTGAGAGG | 198 |
| AGCACGGGTCACAGGAAGTGGGG | 199 |
| CTATGGTGAGAGGCGTCCTGTGG | 200 |
| CAGCACGGGTCACAGGAAGTGGG | 201 |
| ATGGGATGGGGCCCAACAGACGG | 202 |
| CTCCCGCCATGCAGACACCTTGG | 203 |
| GGGGATCCCGGGGACCCCGGGGG | 204 |
| TGTCCGCTGTGGCCTCAGGAGGG | 205 |
| GCAGTTGGGAGGGAACTCTTGGG | 206 |
| GCCTGGCTAGCCTCAGAGGAGGG | 207 |
| GAGAGTTGAGGAGAAACCTATGG | 208 |
| ACCTGTATTTGAGGTTGGCCTGG | 209 |
| TCGGGCAGGCGGCGGGACGCCGG | 210 |
| TAGAGTCCCTCTGGGGACAGGGG | 211 |
| GAAGTGACACTGAAGGGCCTGGG | 212 |
| AGCAGCCTGAGCTGATCTCCTGG | 213 |
| TCATGGCAACTTCCATCTCCTGG | 214 |
| GTCACAGGTTCCTGTCAGAGAGG | 215 |
| CACCAAGGTGTCTGCATGGCGGG | 216 |
| GTAGGTTCTAGAAGGTGACAGGG | 217 |
| AATTGAATTAATAGGACATGGGG | 218 |
| TTCCAAGCACCTGATTTCTGTGG | 219 |
| CTGTCAGAGAGGATGCTCTAGGG | 220 |
| GTGCTGTCCGCTGTGGCCTCAGG | 221 |
| CCCGGGGAGGGAGGATGCTCGGG | 222 |
| GGGGTGGCTCGGGGGGCCCCGGG | 223 |
| GTCAAGTCAGAGCTGGGCCCTGG | 224 |
| GACACTGAAGGGCCTGGGCCTGG | 225 |
| CTGAAAGTGAACCAACACTGTGG | 226 |
| AAAGGGGTACACTGCCTTGGAGG | 227 |
| TCTGGAAACTTCTAAGCATTCGG | 228 |
| AGAGTTGAGGAGAAACCTATGGG | 229 |
| CTCTGAGGCTAGCCAGGCCCAGG | 230 |
| AGGGGTGGCTCGGGGGGCCCCGG | 231 |
| TCCACATTGATTTGCCTTTCTGG | 232 |
| AGGGTGGAAATCTAAGAGACAGG | 233 |
| CTGTCCGCTGTGGCCTCAGGAGG | 234 |
| AGACACAGGACCTGTATTTGAGG | 235 |
| GGGTGGCTCGGGGGGCCCCGGGG | 236 |
| TGGCTGTGCCGCAGGCTTCCAGG | 237 |
| TCCAGAAAGGCAAATCAATGTGG | 238 |
| CACCAGCCTGGAAAGGCACGGGG | 239 |
| CGGGGTCCCCGGGATCCCCGGGG | 240 |
| ACACTGCCTTGGAGGGGCAAAGG | 241 |
| TGACAGGAACCTGTGACCTCAGG | 242 |
| TCTCATGTGGGCTATCAAGATGG | 243 |
| GGGAACTCTTGGGAGGGCCAGGG | 244 |
| GAGGCCACAGCGGACAGCACGGG | 245 |
| TGTCCTATTAATTCAATTCTCGG | 246 |
| AGGGAACTCTTGGGAGGGCCAGG | 247 |
| GCACCTGATTTCTGTGGTATTGG | 248 |
| GTATCTTGAGTGTCTTTTCTCGG | 249 |
| CTGTGGAAAACTTCCTTTATGGG | 250 |
| GGGACCTCCAGCAGATGCAGAGG | 251 |
| CTGGGGACAGGGGTGACCACTGG | 252 |
| TTCAGTGTCACTTCTTTTGGGGG | 253 |
| TCCCTCCTCTGAGGCTAGCCAGG | 254 |
| GGCGCCGGGATCGGGGCCCCCGG | 255 |
| GGTTCTAGAAGGTGACAGGGTGG | 256 |
| TTGGGAGGGAACTCTTGGGAGGG | 257 |
| ATCAGGTGCTTGGAAAGTAGAGG | 258 |
| CGGGGAGGGAGGATGCTCGGGGG | 259 |
| TGAGGCCACAGCGGACAGCACGG | 260 |
| TGGTCACCCCTGTCCCCAGAGGG | 261 |
| TCCCCGCCTCCTGCCCTGCAGGG | 262 |
| GGCTGCCCTGGCTGTGCCGCAGG | 263 |
| TCCAAAAGCTGGCATTGTCAGGG | 264 |
| CAATGCCAGCTTTTGGATGACGG | 265 |
| CTGGGCCTGGCTAGCCTCAGAGG | 266 |
| GGTTCACTTTCAGTCTTTCATGG | 267 |
| AGGAGAAACCTATGGGGGGTGGG | 268 |
| TCTAAAAGACAGCCCAGCCCAGG | 269 |
| CGGGGATCCCGGGGACCCCGGGG | 270 |
| TCTTCTCCAGGGGAACAAAGGGG | 271 |
| ACTGACACAAAAAGTCAGCACGG | 272 |
| CCTGAAGAGTCAGGTCACCAAGG | 273 |
| GGAGGAGAGTCCCACCTGGAAGG | 274 |
| GGGCAGCCACCAGCCTGGAAAGG | 275 |
| AGCCCTATTTCTCTCTCCTCTGG | 276 |
| GCCACCAGCCTGGAAAGGCACGG | 277 |
| TGACACCCTGAGTGTGGCCTCGG | 278 |
| AATTAATAGGACATGGGGAGGGG | 279 |
| GGCTCGGGGGGCCCCGGGGAGGG | 280 |
| TAATAGGACATGGGGAGGGGAGG | 281 |
| CTCTTCTCCAGGGGAACAAAGGG | 282 |
| TCGGGGCCCCCGGGGTCCCCGGG | 283 |
| TCCCTGACAATGCCAGCTTTTGG | 284 |
| GAATTAATAGGACATGGGGAGGG | 285 |
| AATGAGGGGGTCAGAGACACAGG | 286 |
| GAAAACTTCCTTTATGGGGCCGG | 287 |
| CTTGGGAGGGCCAGGGACTTTGG | 288 |
| CCCCTGCAGGGCAGGAGGCGGGG | 289 |
| TCAGTGTCACTTCTTTTGGGGGG | 290 |
| ATCCCCGTTCTTCTTCCTCCTGG | 291 |
| CTTCCTCCTGGCCACAGTGTTGG | 292 |
| TGCAGTTGGGAGGGAACTCTTGG | 293 |
| TGGCTCGGGGGGCCCCGGGGAGG | 294 |
| CTGCAAAGGCCTGAAGAGTCAGG | 295 |
| CCGTGTGCGGAAGACGCCGCCGG | 296 |
| CCCCGGGGAGGGAGGATGCTCGG | 297 |
| TTCCAGGCTGGTGGCTGCCCTGG | 298 |
| AGGTCACCAAGGTGTCTGCATGG | 299 |
| GGCGGCCCCTGCAGGGCAGGAGG | 300 |
| TCGGGGGGCCCCGGGGAGGGAGG | 301 |
| CCAAAAGAAGTGACACTGAAGGG | 302 |
| GGCCAGGAGGAAGAAGAACGGGG | 303 |
| GCCCAGGGTCCTGGTGGGGTAGG | 304 |
| GCTAGCCTCAGAGGAGGGAGTGG | 305 |
| GAGGGTCCTTTGCCCCTCCAAGG | 306 |
| CCACCAGCCTGGAAAGGCACGGG | 307 |
| GATTTCTGTGGTATTGGGGTTGG | 308 |
| CTAGCCTCAGAGGAGGGAGTGGG | 309 |
| CCCGGGGTCCCCGGGATCCCCGG | 310 |
| CATGGGGTGGTAGGTGGAGTGGG | 311 |
| AATGCCAGCTTTTGGATGACGGG | 312 |
| GCCCCTGCAGGGCAGGAGGCGGG | 313 |
| GAGGAGAAACCTATGGGGGGTGG | 314 |
| AGAAGTGACACTGAAGGGCCTGG | 315 |
| CCTCCAGCAGATGCAGAGGAAGG | 316 |
| CCTCTTCTCCAGGGGAACAAAGG | 317 |
| CCGGGGTCCCCGGGATCCCCGGG | 318 |
| TAGCCTCAGAGGAGGGAGTGGGG | 319 |
| CAGAGGAAGGGGATGCAGTTGGG | 320 |
| CTCCAGCAGATGCAGAGGAAGGG | 321 |
| GATTCTGTTTTTCCTCTGCCTGG | 322 |
| CTTCAGTGTCACTTCTTTTGGGG | 323 |
| CATAGAGTCCCTCTGGGGACAGG | 324 |
| GGACCCTCCTGAGGCCACAGCGG | 325 |
| CCATGGGGTGGTAGGTGGAGTGG | 326 |
| GACACCCTGAGTGTGGCCTCGGG | 327 |
| ATGCTTAGAAGTTTCCAGAAAGG | 328 |
| AGCTGGGCCCTGGAAGCCTGCGG | 329 |
| TACCACAGAAATCAGGTGCTTGG | 330 |
| ACCCCAATACCACAGAAATCAGG | 331 |
| TTCTACAGGTAAAAAAACTAAGG | 332 |
| GGCCCCTGCAGGGCAGGAGGCGG | 333 |
| CTCCCCGCCTCCTGCCCTGCAGG | 334 |
| TCTCTGACCCCCTCATTTGTGGG | 335 |
| GGAGAAACCTATGGGGGGTGGGG | 336 |
| ACAGCCCAGCCCAGGACAGACGG | 337 |
| CCTGTATTTGAGGTTGGCCTGGG | 338 |
| AGCCAGGGCAGCCACCAGCCTGG | 339 |
| AGCCTCAGAGGAGGGAGTGGGGG | 340 |
| GTTCAGTGTTTCACTCATCCAGG | 341 |
| CTGACTCTTCAGGCCTTTGCAGG | 342 |
| ATCCCCCACTCCCTCCTCTGAGG | 343 |
| CCCAAAAGAAGTGACACTGAAGG | 344 |
| TGGCCAGGAGGAAGAAGAACGGG | 345 |
| GGGAGGAAGGTTATGGGATGGGG | 346 |
| CCTGAGGCTTCCTGCACTCTAGG | 347 |
| TAGTTTTTTTACCTGTAGAATGG | 348 |
| AAGTGGGGTAGGGAACAAGGTGG | 349 |
| GGGTCACAGGAAGTGGGGTAGGG | 350 |
| CACCTGATTTCTGTGGTATTGGG | 351 |
| TTTGCAACTGGGTCTCATGTGGG | 352 |
| GAGAAACCTATGGGGGGTGGGGG | 353 |
| GAGGGAGGAGGGGTGGCTCGGGG | 354 |
| GCCTGTAATCCCACAAATGAGGG | 355 |
| GCAGAGGAAGGGGATGCAGTTGG | 356 |
| ACCTGATTTCTGTGGTATTGGGG | 357 |
| AAACCAGAGGAGAGAGAAATAGG | 358 |
| AACCAGAGGAGAGAGAAATAGGG | 359 |
| GAGGAGAGAGAAATAGGGCTTGG | 360 |
| CTGCAGGGCAGGAGGCGGGGAGG | 361 |
| AGGAAGGGGATGCAGTTGGGAGG | 362 |
| GGTATTGGGGTTGGAACCTGAGG | 363 |
| TTTTGCAACTGGGTCTCATGTGG | 364 |
| GGGAGGAGGGGTGGCTCGGGGGG | 365 |
| TCCCCTTCCTCTGCATCTGCTGG | 366 |
| AGGAAGTGGGGTAGGGAACAAGG | 367 |
| AGGGGAGGAAGGTTATGGGATGG | 368 |
| CCTGTAATCCCACAAATGAGGGG | 369 |
| AGTTTTTTTACCTGTAGAATGGG | 370 |
| AGGAAGAAGAACGGGGATGGGGG | 371 |
| TGCAGGGCAGGAGGCGGGGAGGG | 372 |
| GGAAGGGGATGCAGTTGGGAGGG | 373 |
| CAAAGTCACTGTGTAGATGAAGG | 374 |
| GTGGCCAGGAGGAAGAAGAACGG | 375 |
| TCCAGCAGATGCAGAGGAAGGGG | 376 |
| AGGGAGGAGGGGTGGCTCGGGGG | 377 |
| AACCTATGGGGGGTGGGGGTGGG | 378 |
| GGGGAGGGGAGGAAGGTTATGGG | 379 |
| CACCCACCCCCACCCCCCATAGG | 380 |
| AAACCTATGGGGGGTGGGGGTGG | 381 |
| GGAGGAAGAAGAACGGGGATGGG | 382 |
| GAGGAAGAAGAACGGGGATGGGG | 383 |
| GGAGGGAGGAGGGGTGGCTCGGG | 384 |
| GGTTGGCCTGGGCTACACAGGGG | 385 |
| AGAGGAGGGAGTGGGGGATTGGG | 386 |
| GAGGTTGGCCTGGGCTACACAGG | 387 |
| AGGTTGGCCTGGGCTACACAGGG | 388 |
| TGGGGAGGGGAGGAAGGTTATGG | 389 |
| GAAAGTAGAGGCAGGAGGGTTGG | 390 |
| GAGGAGGGAGTGGGGGATTGGGG | 391 |
| GGGGAGGAAGGTTATGGGATGGG | 392 |
| AGAGTGCTTGCCTAGAGTGCAGG | 393 |
| AGGAGGGAGTGGGGGATTGGGGG | 394 |
| CAGAGGAGGGAGTGGGGGATTGG | 395 |
| AGGAGGAAGAAGAACGGGGATGG | 396 |
| TTTTTTCCCCTGTGTAGCCCAGG | 397 |
| GGTGCTTGGAAAGTAGAGGCAGG | 398 |
| CTTGGAAAGTAGAGGCAGGAGGG | 399 |
| CTGTAATCCCACAAATGAGGGGG | 400 |
| AGGACATGGGGAGGGGAGGAAGG | 401 |
| TCATCTACACAGTGACTTTGAGG | 402 |
| GAGGGAGTGGGGGATTGGGGGGG | 403 |
| GGGAGGGAGGAGGGGTGGCTCGG | 404 |
| AGGGAGTGGGGGATTGGGGGGGG | 405 |
| AACAACAAAAACAAAACCAGAGG | 406 |
| CTATGGGGGGTGGGGGTGGGTGG | 407 |
| GGAGGGAGTGGGGGATTGGGGGG | 408 |
| TGCCTGTAATCCCACAAATGAGG | 409 |
| GAGTGGGGGATTGGGGGGGGGGG | 410 |
| AGGGCAGGAGGCGGGGAGGGAGG | 411 |
| CAGGAGGCGGGGAGGGAGGAGGG | 412 |
| AGGAGGCGGGGAGGGAGGAGGGG | 413 |
| GGAGTGGGGGATTGGGGGGGGGG | 414 |
| GGGAGTGGGGGATTGGGGGGGGG | 415 |
| GCAGGAGGCGGGGAGGGAGGAGG | 416 |
| AGGCGGGGAGGGAGGAGGGGTGG | 417 |
| GCTTGGAAAGTAGAGGCAGGAGG | 418 |
| GAGAGAGAGAGAGAGAGTTGAGG | 419 |

It is to be understood that the description, specific examples and data, while indicating exemplary embodiments, are given by way of illustration and are not intended to limit the present inventions. Various changes and modifications within the present inventions, including combining embodiments in whole and in part, will become apparent to the skilled artisan from the discussion, disclosure and data contained herein, and thus are considered part of the inventions.

1-34. (canceled)

35. A method for making at least one protein of interest, comprising:
(a) providing a mammalian cell comprising a first Stable Integration Site located in a first Genomic Safe Harbor and a second Stable Integration Site that is not located in the first Genomic Safe Harbor, wherein the first Stable Integration Site comprises a first reporter gene encoding a first reporter protein and the second Stable Integration Site comprises a second reporter gene encoding a second reporter protein, wherein the first reporter protein and the second reporter protein are different, and wherein the first and second Stable Integration Sites comprise recombinase recognition sites;
(b) introducing a DNA polynucleotide encoding the protein of interest into a Stable Integration Site by recombinase mediated cassette exchange, and
(c) culturing the mammalian cell under conditions that allow expression of the DNA polynucleotide encoding the protein of interest.

36. The method according to claim 35, wherein the mammalian cell is a Human Amniotic Epithelial Cell.

37. The method according to claim 35, wherein the mammalian cell is a HEK293 cell.

38. The method according to claim 35, wherein the mammalian cell is a CHO cell.

39. The method according to claim 35, wherein the first Stable Integration Site comprises a first DNA polynucleotide encoding a first protein and the second Stable Integration Site comprises a second DNA polynucleotide encoding a second protein.

40. The method according to claim 39, wherein the first protein is a viral protein.

41. The method according to claim 39, wherein the second protein is a viral protein.

42. The method according to claim 35, wherein the mammalian cell comprises a DNA polynucleotide encoding an adeno-associated virus protein and a DNA polynucleotide encoding an adenovirus protein.

43. The method according to claim 35, wherein the second Stable Integration Site is located in a second Genomic Safe Harbor that is different from the first Genomic Safe Harbor.

44. A method of creating a mammalian cell with multiple Stable Integration Sites, wherein the method comprises:
(A) providing a mammalian cell comprising a first DNA cassette comprising in 5′ to 3′ order a DNA polynucleotide encoding the first lox site, a promoter, a selection marker gene encoding a selection marker protein, an IRES, a first reporter gene encoding a first reporter protein, a promoter operably linked to an operator, a Cas9 gene and the second lox site;
(B) integrating a second DNA cassette comprising in a 5′ to 3′ order a DNA polynucleotide comprising a first Genomic Safe Harbor homology arm containing a CRISPR sgRNA target site, a third lox site, a second reporter gene encoding a second reporter protein, a forth lox site and a second Genomic Safe Harbor homology arm containing an CRISPR sgRNA target site, wherein the first lox site, the second lox site, the third lox site and the forth lox site are different, wherein the first and second guide arms can contain a region with alterations, and wherein the second reporter protein is different from the first reporter protein;
(C) exchanging the first DNA cassette with a third DNA cassette, wherein the third DNA cassette comprises in a 5′ to 3′ order a DNA polynucleotide e encoding the first lox site, a promoter, a third reporter gene encoding a third reporter protein, and the second lox site, wherein the third reporter protein is different from the second reporter protein, thereby providing the mammalian cell with multiple Stable Integration Sites.

45. The method according to claim 44, wherein the alterations prevent recreating a targetable site.

46. The method according to claim 44, wherein the mammalian cell is a HEK293 Cell.

47. The method according to claim 44, wherein the mammalian cell is a CHO Cell.

48. The method according to claim 44, wherein the cell of step (A) further comprises a DNA polynucleotide encoding a repressor under the control of a CMV promoter.

49. The method according to claim 44, wherein the cell of step (B) further comprises a DNA polynucleotide encoding a repressor under the control of a CMV promoter.

50. The method according to claim 44, wherein the cell of step (C) further comprises a DNA polynucleotide encoding a repressor under the control of a CMV promoter.

51-89. (canceled)

90. The method according to claim 35, wherein the Genomic Safe Harbor is an AAVS1-like region.

91. The method according to claim 44, wherein the first Genomic Safe Harbor homology arm and the second Genomic Safe Harbor homology arm have homology to an AAVS1-like region.

92. The method according to claim 44, wherein a DNA polynucleotide of interest can be integrated into a Stable Integration Site.

93. the method according to claim 92, therein the DNA polynucleotide of interest encodes a polypeptide of interest.
